(19) United States
(12) Patent Application Publication
(10) Pub. No.: US 2023/0159496 A1
(43) Pub. Date: May 25, 2023

(54) COMPOUNDS AND METHODS FOR MODULATING SPLICING

(71) Applicant: REMIX THERAPEUTICS INC., Cambridge, MA (US)

(72) Inventors: Dominic Reynolds, Stoneham, MA (US); Serge Leger, Notre-Dame-de-l'Île-Perrot (CA); Michael W. Seiler, Belmont, MA (US); Anant A. Agrawal, Waltham, MA (US); Frederic Vaillancourt, Newton, MA (US); Peter Smith, Arlington, MA (US); Allen T. Hopper, Lexington, MA (US); Sudeep Prajapati, Somerville, MA (US); Olivier Soueidan, Vaudreuil-Dorion (CA)

(21) Appl. No.: 17/917,729

(22) PCT Filed: Apr. 8, 2021
(86) PCT No.: PCT/US2021/026481

§ 371 Date: Oct. 7, 2022

Related U.S. Application Data
(60) Provisional application No. 63/126,494, filed on Dec. 16, 2020.
(60) Provisional application No. 63/072,922, filed on Aug. 31, 2020.
(60) Provisional application No. 63/044,318, filed on Jun. 25, 2020.
(60) Provisional application No. 63/007,331, filed on Apr. 8, 2020.

(51) Int. Cl. C07D 401/14; C07D 471/04; C07D 519/00; C07D 403/14; C07D 487/04; C07D 471/08; C07D 413/14; C07D 417/14

(52) U.S. Cl. CPC C07D 401/14; C07D 471/04; C07D 519/00; C07D 403/14; C07D 487/04; C07D 471/08; C07D 413/14; C07D 417/14

(57) ABSTRACT

The present disclosure features compounds and related compositions that, inter alia, modulate nucleic acid splicing, e.g., splicing of a pre-mRNA, as well as methods of use thereof.

### CLAIM OF PRIORITY

This application claims priority to U.S. Application No. 63/007,331, filed Apr. 8, 2020; U.S. Application No. 63/044,318, filed Jun. 25, 2020; U.S. Application No. 63/072,922, filed Aug. 31, 2020; and U.S. Application No. 63/126,494, filed Dec. 16, 2020. The disclosure of each of the foregoing applications is incorporated herein by reference in its entirety.

### BACKGROUND

Alternative splicing is a major source of protein diversity in higher eukaryotes and is frequently regulated in a tissue-specific or development stage-specific manner. Disease associated alternative splicing patterns in pre-mRNAs are often mapped to changes in splice site signals or sequence motifs and regulatory splicing factors (Faustino and Cooper (2003), Genes Dev 17(4):419-37). Current therapies to modulate RNA expression involve oligonucleotide targeting and gene therapy; however, each of these modalities exhibit unique challenges as currently presented. As such, there is a need for new technologies to modulate RNA expression, including the development of small molecule compounds that target splicing.

### SUMMARY

The present disclosure features compounds and related compositions that, inter alia, modulate nucleic acid splicing, e.g., splicing of a pre-mRNA, as well as methods of use thereof. In an embodiment, the compounds described herein are compounds of Formulas (I), (III), or (V) and pharmaceutically acceptable salts, solvates, hydrates, tautomers, or stereoisomers thereof. The present disclosure additionally provides methods of using the compounds of the disclosure (e.g., compounds of Formulas (I), (III), or (V) and pharmaceutically acceptable salts, solvates, hydrates, tautomers, stereoisomers thereof), and compositions thereof, e.g., to target, and in embodiments bind or form a complex with, a nucleic acid (e.g., a pre-mRNA or nucleic acid component of a small nuclear ribonucleoprotein (snRNP) or spliceosome), a protein (e.g., a protein component of an snRNP or spliceosome, e.g., a member of the splicing machinery, e.g., one or more of the U1, U2, U4, U5, U6, U11, U12, U4atac, U6atac snRNPs), or a combination thereof. In another aspect, the compounds described herein may be used to alter the composition or structure of a nucleic acid (e.g., a pre-mRNA or mRNA (e.g., a pre-mRNA and the mRNA which arises from the pre-mRNA), e.g., by increasing or decreasing splicing at a splice site. In some embodiments, increasing or decreasing splicing results in modulating the level of a gene product (e.g., an RNA or protein) produced.

In another aspect, the compounds described herein may be used for the prevention and/or treatment of a disease, disorder, or condition, e.g., a disease, disorder or condition associated with splicing, e.g., alternative splicing. In some embodiments, the compounds described herein (e.g., compounds of Formulas (I), (III), or (V), and pharmaceutically acceptable salts, solvates, hydrates, tautomers, stereoisomers thereof) and compositions thereof are used for the prevention and/or treatment of a proliferative disease, disorder, or condition (e.g., a disease, disorder, or condition characterized by unwanted cell proliferation, e.g., a cancer or a benign neoplasm) in a subject. In some embodiments, the compounds described herein (e.g., compounds of Formulas (I), (III), or (V), and pharmaceutically acceptable salts, solvates, hydrates, tautomers, stereoisomers thereof) and compositions thereof are used for the prevention and/or treatment of a non-proliferative disease, disorder, or condition. In some embodiments, the compounds described herein (e.g., compounds of Formulas (I), (III), or (V), and pharmaceutically acceptable salts, solvates, hydrates, tautomers, stereoisomers thereof) and compositions thereof are used for the prevention and/or treatment of a neurological disease or disorder, an autoimmune disease or disorder, immunodeficiency disease or disorder, a lysosomal storage disease or disorder, a cardiovascular disease or disorder, a metabolic disease or disorder, a respiratory disease or disorder, a renal disease or disorder, or an infectious disease in a subject.

In one aspect, the present disclosure provides compounds of Formula (I):

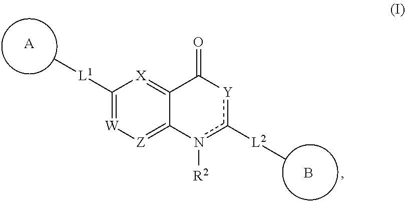

or a pharmaceutically acceptable salt, solvate, hydrate, tautomer, or stereoisomer thereof, wherein each of A, B, L¹, L², W, X, Y, Z, R², and subvariables thereof are defined as described herein.

In another aspect, the present disclosure provides compounds of Formula (III):

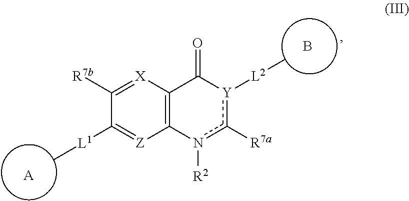

or a pharmaceutically acceptable salt, solvate, hydrate, tautomer, or stereoisomer thereof, wherein each of A, B, L¹, L², X, Y, Z, R², R^{7a}, R^{7b}, and subvariables thereof are defined as described herein.

In another aspect, the present disclosure provides compounds of Formula (V):

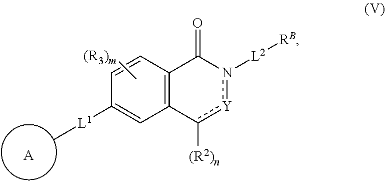

or a pharmaceutically acceptable salt, solvate, hydrate, tautomer, or stereoisomer thereof, wherein each of A, R^{B}, L¹, L², Y, R², R³, m, n, and subvariables thereof are defined as described herein.

In another aspect, the present invention provides pharmaceutical compositions comprising a compound of Formulas (I), (III), or (V), or a pharmaceutically acceptable salt, solvate, hydrate, tautomer, or stereoisomer thereof, and optionally a pharmaceutically acceptable excipient. In an embodiment, the pharmaceutical compositions described herein include an effective amount (e.g., a therapeutically effective amount) of a compound of Formulas (I), (III), or (V), or a pharmaceutically acceptable salt, solvate, hydrate, tautomer, or stereoisomer thereof.

In another aspect, the present disclosure provides methods for modulating splicing, e.g., splicing of a nucleic acid (e.g., a DNA or RNA, e.g., a pre-mRNA) with a compound of Formulas (I), (III), or (V), or a pharmaceutically acceptable salt, solvate, hydrate, tautomer, or stereoisomer thereof. In another aspect, the present disclosure provides compositions for use in modulating splicing, e.g., splicing of a nucleic acid (e.g., a DNA or RNA, e.g., a pre-mRNA) with a compound of Formulas (I), (III), or (V), or a pharmaceutically acceptable salt, solvate, hydrate, tautomer, or stereoisomer thereof. Modulation of splicing may comprise impacting any step involved in splicing and may include an event upstream or downstream of a splicing event. For example, in some embodiments, the compound of Formulas (I), (III), or (V) binds to a target, e.g., a target nucleic acid (e.g., DNA or RNA, e.g., a precursor RNA, e.g., a pre-mRNA), a target protein, or combination thereof (e.g., an snRNP and a pre-mRNA). A target may include a splice site in a pre-mRNA or a component of the splicing machinery, such as the U1 snRNP. In some embodiments, the compound of Formulas (I), (III), or (V) alters a target nucleic acid (e.g., DNA or RNA, e.g., a precursor RNA, e.g., a pre-mRNA), target protein, or combination thereof. In some embodiments, the compound of Formulas (I), (III), or (V) increases or decreases splicing at a splice site on a target nucleic acid (e.g., an RNA, e.g., a precursor RNA, e.g., a pre-mRNA) by about 0.5% or more (e.g., about 1%, 2%, 3%, 4%, 5%, 10%, 20%, 30%, 40%, 50%, 75%, 90%, 95%, or more), relative to a reference (e.g., the absence of a compound of Formulas (I), (III), or (V), e.g., in a healthy or diseased cell or tissue). In some embodiments, the presence of a compound of Formulas (I), (III), or (V) results an increase or decrease of transcription of a target nucleic acid (e.g., an RNA) by about 0.5% or more (e.g., about 1%, 2%, 3%, 4%, 5%, 10%, 20%, 30%, 40%, 50%, 75%, 90%, 95%, or more), relative to a reference (e.g., the absence of a compound of Formulas (I), (III), or (V), e.g., in a healthy or diseased cell or tissue).

In another aspect, the present disclosure provides methods for preventing and/or treating a disease, disorder, or condition in a subject by administering a compound of Formulas (I), (III), or (V), or a pharmaceutically acceptable salt, solvate, hydrate, tautomer, or stereoisomer thereof, or related compositions. In some embodiments, the disease or disorder entails unwanted or aberrant splicing. In some embodiments, the disease or disorder is a proliferative disease, disorder, or condition. Exemplary proliferative diseases include cancer, a benign neoplasm, or angiogenesis. In other embodiments, the present disclosure provides methods for treating and/or preventing a non-proliferative disease, disorder, or condition. In still other embodiments, the present disclosure provides methods for treating and/or preventing a neurological disease or disorder, autoimmune disease or disorder, immunodeficiency disease or disorder, lysosomal storage disease or disorder, cardiovascular disease or disorder, metabolic disease or disorder, respiratory disease or disorder, renal disease or disorder, or infectious disease.

In another aspect, the present disclosure provides methods of down-regulating the expression of (e.g., the level of or the rate of production of) a target protein with a compound of Formulas (I), (III), or (V), or a pharmaceutically acceptable salt, solvate, hydrate, tautomer, or stereoisomer thereof in a biological sample or subject. In another aspect, the present disclosure provides methods of up-regulating the expression of (e.g., the level of or the rate of production of) a target protein with a compound of Formulas (I), (III), or (V), or a pharmaceutically acceptable salt, solvate, hydrate, tautomer, or stereoisomer thereof in a biological sample or subject. In another aspect, the present disclosure provides methods of altering the isoform of a target protein with a compound of Formulas (I), (III), or (V), or a pharmaceutically acceptable salt, solvate, hydrate, tautomer, or stereoisomer thereof in a biological sample or subject. Another aspect of the disclosure relates to methods of inhibiting the activity of a target protein in a biological sample or subject. In some embodiments, administration of a compound of Formulas (I), (III), or (V) to a biological sample, a cell, or a subject comprises inhibition of cell growth or induction of cell death.

In another aspect, the present disclosure provides compositions for use in preventing and/or treating a disease, disorder, or condition in a subject by administering a compound of Formulas (I), (III), or (V) or a pharmaceutically acceptable salt, solvate, hydrate, tautomer, or stereoisomer thereof, or related compositions. In some embodiments, the disease or disorder entails unwanted or aberrant splicing. In some embodiments, the disease or disorder is a proliferative disease, disorder, or condition. Exemplary proliferative diseases include cancer, a benign neoplasm, or angiogenesis. In other embodiments, the present disclosure provides methods for treating and/or preventing a non-proliferative disease, disorder, or condition. In still other embodiments, the present disclosure provides compositions for use in treating and/or preventing a neurological disease or disorder, autoimmune disease or disorder, immunodeficiency disease or disorder, lysosomal storage disease or disorder, cardiovascular disease or disorder, metabolic disease or disorder, respiratory disease or disorder, renal disease or disorder, or infectious disease.

In another aspect, the present disclosure provides compositions for use in down-regulating the expression of (e.g., the level of or the rate of production of) a target protein with a compound of Formulas (I), (III), or (V), or a pharmaceutically acceptable salt, solvate, hydrate, tautomer, or stereoisomer thereof in a biological sample or subject. In another aspect, the present disclosure provides compositions for use in up-regulating the expression of (e.g., the level of or the rate of production of) a target protein with a compound of Formulas (I), (III), or (V), or a pharmaceutically acceptable salt, solvate, hydrate, tautomer, or stereoisomer thereof in a biological sample or subject. In another aspect, the present disclosure provides compositions for use in altering the isoform of a target protein with a compound of Formulas (I), (III), or (V), or a pharmaceutically acceptable salt, solvate, hydrate, tautomer, or stereoisomer thereof in a biological sample or subject. Another aspect of the disclosure relates to compositions for use in inhibiting the activity of a target protein in a biological sample or subject. In some embodiments, administration of a compound of Formulas (I), (III), or (V) to a biological sample, a cell, or a subject comprises inhibition of cell growth or induction of cell death.

In another aspect, the present disclosure features kits comprising a container with a compound of Formulas (I), (III), or (V), or a pharmaceutically acceptable salt, solvate, hydrate, tautomer, stereoisomer thereof, or a pharmaceutical composition thereof. In certain embodiments, the kits described herein further include instructions for administering the compound of Formulas (I), (III), or (V), or the pharmaceutically acceptable salt, solvate, hydrate, tautomer, stereoisomer thereof, or the pharmaceutical composition thereof.

In any and all aspects of the present disclosure, in some embodiments, the compound, target nucleic acid (e.g., DNA, RNA, e.g., pre-mRNA), or target protein described herein is a compound, target nucleic acid (e.g., DNA, RNA, e.g., pre-mRNA), or target protein other than a compound, target nucleic acid (e.g., DNA, RNA, e.g., pre-mRNA), or target protein described one of U.S. Pat. No. 8,729,263, U.S. Publication No. 2015/0005289, WO 2014/028459, WO 2016/128343, WO 2016/196386, WO 2017/100726, WO 2018/232039, WO 2018/098446, WO 2019/028440, WO 2019/060917, WO 2019/199972, and WO 2020/004594. In some embodiments, the compound, target nucleic acid (e.g., DNA, RNA, e.g., pre-mRNA), or target protein described herein is a compound, target nucleic acid (e.g., DNA, RNA, e.g., pre-mRNA), or target protein described one of U.S. Pat. No. 8,729,263, U.S. Publication No. 2015/0005289, WO 2014/028459, WO 2016/128343, WO 2016/196386, WO 2017/100726, WO 2018/232039, WO 2018/098446, WO 2019/028440, WO 2019/060917, WO 2019/199972, and WO 2020/004594, each of which is incorporated herein by reference in its entirety.

The details of one or more embodiments of the invention are set forth herein. Other features, objects, and advantages of the invention will be apparent from the Detailed Description, the Examples, and the Claims.

### DETAILED DESCRIPTION

### Selected Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

The abbreviations used herein have their conventional meaning within the chemical and biological arts. The chemical structures and formulae set forth herein are constructed according to the standard rules of chemical valency known in the chemical arts.

When a range of values is listed, it is intended to encompass each value and sub—range within the range. For example “C₁-C₆ alkyl” is intended to encompass, C₁, C₂, C₃, C₄, C₅, C₆, C₁-C₆, C₁-C₄, C₁-C₃, C₁-C₂, C₂-C₆, C₂-C₄, C₂-C₃, C₃-C₆, C₃-C₄, C₄-C₆, C₄-C₅, and C₅-C₆ alkyl.

The following terms are intended to have the meanings presented therewith below and are useful in understanding the description and intended scope of the present invention.

As used herein, “alkyl” refers to a radical of a straight—chain or branched saturated hydrocarbon group having from 1 to 24 carbon atoms (“C₁-C₂₄ alkyl”). In some embodiments, an alkyl group has 1 to 12 carbon atoms (“C₁-C₁₂ alkyl”). In some embodiments, an alkyl group has 1 to 8 carbon atoms (“C₁-C₈ alkyl”). In some embodiments, an alkyl group has 1 to 6 carbon atoms (“C₁-C₆ alkyl”). In some embodiments, an alkyl group has 2 to 6 carbon atoms (“C₂-C₆ alkyl”). In some embodiments, an alkyl group has 1 carbon atom (“C₁ alkyl”). Examples of C₁-C₆alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Each instance of an alkyl group may be independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent. In certain embodiments, the alkyl group is unsubstituted C₁-C₁₀ alkyl (e.g., —CH₃). In certain embodiments, the alkyl group is substituted C₁-C₆ alkyl.

As used herein, “alkenyl” refers to a radical of a straight—chain or branched hydrocarbon group having from 2 to 24 carbon atoms, one or more carbon-carbon double bonds, and no triple bonds (“C₂-C₂₄ alkenyl”). In some embodiments, an alkenyl group has 2 to 10 carbon atoms (“C₂-C₁₀ alkenyl”). In some embodiments, an alkenyl group has 2 to 8 carbon atoms (“C₂-C₈ alkenyl”). In some embodiments, an alkenyl group has 2 to 6 carbon atoms (“C₂-C₆ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂-C₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂-C₆ alkenyl groups include the aforementioned C₂-C₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Each instance of an alkenyl group may be independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent. In certain embodiments, the alkenyl group is unsubstituted C₁-C₁₀ alkenyl. In certain embodiments, the alkenyl group is substituted C₂-C₆ alkenyl.

As used herein, the term “alkynyl” refers to a radical of a straight—chain or branched hydrocarbon group having from 2 to 24 carbon atoms, one or more carbon-carbon triple bonds (“C₂-C₂₄ alkenyl”). In some embodiments, an alkynyl group has 2 to 10 carbon atoms (“C₂-C₁₀ alkynyl”). In some embodiments, an alkynyl group has 2 to 8 carbon atoms (“C₂-C₈ alkynyl”). In some embodiments, an alkynyl group has 2 to 6 carbon atoms (“C₂-C₆ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂-C₄ alkynyl groups include ethynyl (C₂), 1-propynyl (C₃), 2-propynyl (C₃), 1-butynyl (C₄), 2-butynyl (C₄), and the like. Each instance of an alkynyl group may be independently optionally substituted, i.e., unsubstituted (an “unsubstituted alkynyl”) or substituted (a “substituted alkynyl”) with one or more substituents e.g., for instance from 1 to 5 substituents, 1 to 3 substituents, or 1 substituent. In certain embodiments, the alkynyl group is unsubstituted C₂₋₁₀ alkynyl. In certain embodiments, the alkynyl group is substituted C₂₋₆ alkynyl.

As used herein, the term “haloalkyl,” refers to a non-cyclic stable straight or branched chain, or combinations thereof, including at least one carbon atom and at least one halogen selected from the group consisting of F, Cl, Br, and I. The halogen(s) F, Cl, Br, and I may be placed at any position of the haloalkyl group. Exemplary haloalkyl groups include, but are not limited to: —CF₃, —CCl₃, —CH₂—CF₃, —CH₂—CCl₃, —CH₂—CBr3, —CH₂—Cl₃, —CH₂—CH₂—CH(CF₃)—CH₃, —CH₂—CH₂—CH(Br)—CH₃, and —CH₂—CH═CH—CH₂—CF₃. Each instance of a haloalkyl group may be independently optionally substituted, i.e., unsubstituted (an “unsubstituted haloalkyl”) or substituted (a “substituted haloalkyl”) with one or more substituents e.g., for instance from 1 to 5 substituents, 1 to 3 substituents, or 1 substituent.

As used herein, the term “heteroalkyl,” refers to a non-cyclic stable straight or branched chain, or combinations thereof, including at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) O, N, P, S, and Si may be placed at any position of the heteroalkyl group. Exemplary heteroalkyl groups include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, and —O—CH₂—CH₃. Up to two or three heteroatoms may be consecutive, such as, for example, —CH₂—NH—OCH₃ and —CH₂—O—Si(CH₃)₃. Where “heteroalkyl” is recited, followed by recitations of specific heteroalkyl groups, such as —CH₂O, —NR^{C}R^{D}, or the like, it will be understood that the terms heteroalkyl and —CH₂O or —NR^{C}R^{D} are not redundant or mutually exclusive. Rather, the specific heteroalkyl groups are recited to add clarity. Thus, the term “heteroalkyl” should not be interpreted herein as excluding specific heteroalkyl groups, such as —CH₂O, —NR^{C}R^{D}, or the like. Each instance of a heteroalkyl group may be independently optionally substituted, i.e., unsubstituted (an “unsubstituted heteroalkyl”) or substituted (a “substituted heteroalkyl”) with one or more substituents e.g., for instance from 1 to 5 substituents, 1 to 3 substituents, or 1 substituent.

As used herein, “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆-C₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). An aryl group may be described as, e.g., a C₆-C₁₀-membered aryl, wherein the term “membered” refers to the non-hydrogen ring atoms within the moiety. Aryl groups include phenyl, naphthyl, indenyl, and tetrahydronaphthyl. Each instance of an aryl group may be independently optionally substituted, i.e., unsubstituted (an “unsubstituted aryl”) or substituted (a “substituted aryl”) with one or more substituents. In certain embodiments, the aryl group is unsubstituted C₆-C₁₄ aryl. In certain embodiments, the aryl group is substituted C₆-C₁₄ aryl.

As used herein, “heteroaryl” refers to a radical of a 5-10 membered monocyclic or bicyclic 4n+2 aromatic ring system (e.g., having 6 or 10 π C electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl). A heteroaryl group may be described as, e.g., a 6-10-membered heteroaryl, wherein the term “membered” refers to the non-hydrogen ring atoms within the moiety. Each instance of a heteroaryl group may be independently optionally substituted, i.e., unsubstituted (an “unsubstituted heteroaryl”) or substituted (a “substituted heteroaryl”) with one or more substituents e.g., for instance from 1 to 5 substituents, 1 to 3 substituents, or 1 substituent.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Other exemplary heteroaryl groups include heme and heme derivatives.

As used herein, “cycloalkyl” refers to a radical of a non-aromatic cyclic hydrocarbon group having from 3 to 10 ring carbon atoms (“C₃-C₁₀ cycloalkyl”) and zero heteroatoms in the non-aromatic ring system. In some embodiments, a cycloalkyl group has 3 to 8 ring carbon atoms (“C₃-C₈ cycloalkyl”). In some embodiments, a cycloalkyl group has 3 to 6 ring carbon atoms (“C₃-C₆ cycloalkyl”). In some embodiments, a cycloalkyl group has 3 to 6 ring carbon atoms (“C₃-C₆ cycloalkyl”). In some embodiments, a cycloalkyl group has 5 to 10 ring carbon atoms (“C₅-C₁₀ cycloalkyl”). A cycloalkyl group may be described as, e.g., a C₄-C₇-membered cycloalkyl, wherein the term “membered” refers to the non-hydrogen ring atoms within the moiety. Exemplary C₃-C₆ cycloalkyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃-C₈ cycloalkyl groups include, without limitation, the aforementioned C₃-C₆ cycloalkyl groups as well as cycloheptyl (C₇), cycloheptenyl (C₇), cycloheptadienyl (C₇), cycloheptatrienyl (C₇), cyclooctyl (C₈), cyclooctenyl (C₈), cubanyl (C₈), bicyclo[1.1.1]pentanyl (C₅), bicyclo[2.2.2]octanyl (C₈), bicyclo[2.1.1]hexanyl (C₆), bicyclo[3.1.1]heptanyl (C₇), and the like. Exemplary C₃-C₁₀ cycloalkyl groups include, without limitation, the aforementioned C₃-C₈ cycloalkyl groups as well as cyclononyl (C₉), cyclononenyl (C₉), cyclodecyl (C₁₀), cyclodecenyl (C₁₀), octahydro-1H—indenyl (C₉), decahydronaphthalenyl (C₁₀), spiro[4.5]decanyl (C₁₀), and the like. As the foregoing examples illustrate, in certain embodiments, the cycloalkyl group is either monocyclic (“monocyclic cycloalkyl”) or contain a fused, bridged or spiro ring system such as a bicyclic system (“bicyclic cycloalkyl”) and can be saturated or can be partially unsaturated. “Cycloalkyl” also includes ring systems wherein the cycloalkyl ring, as defined above, is fused with one or more aryl groups wherein the point of attachment is on the cycloalkyl ring, and in such instances, the number of carbons continue to designate the number of carbons in the cycloalkyl ring system. Each instance of a cycloalkyl group may be independently optionally substituted, i.e., unsubstituted (an “unsubstituted cycloalkyl”) or substituted (a “substituted cycloalkyl”) with one or more substituents. In certain embodiments, the cycloalkyl group is unsubstituted C₃-C₁₀ cycloalkyl. In certain embodiments, the cycloalkyl group is a substituted C₃-C₁₀ cycloalkyl.

“Heterocyclyl” as used herein refers to a radical of a 3— to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more cycloalkyl groups wherein the point of attachment is either on the cycloalkyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. A heterocyclyl group may be described as, e.g., a 3-7-membered heterocyclyl, wherein the term “membered” refers to the non-hydrogen ring atoms, i.e., carbon, nitrogen, oxygen, sulfur, boron, phosphorus, and silicon, within the moiety. Each instance of heterocyclyl may be independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl (e.g., 2,2,6,6-tetramethylpiperidinyl), tetrahydropyranyl, dihydropyridinyl, pyridinonyl (e.g., 1-methylpyridin2-onyl), and thianyl. Exemplary 6-membered heterocyclyl groups containing two heteroatoms include, without limitation, piperazinyl, morpholinyl, pyridazinonyl (2-methylpyridazin-3-onyl), pyrimidinonyl (e.g., 1-methylpyrimidin-2-onyl, 3-methylpyrimidin-4-onyl), dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclyl ring) include, without limitation, indolinyl, isoindolinyl, dihydrobenzofuranyl, dihydrobenzothienyl, benzoxazolinonyl, and the like. Exemplary 5-membered heterocyclyl groups fused to a heterocyclyl ring (also referred to herein as a 5,5-bicyclic heterocyclyl ring) include, without limitation, octahydropyrrolopyrrolyl (e.g., octahydropyrrolo[3,4-c]pyrrolyl), and the like. Exemplary 6-membered heterocyclyl groups fused to a heterocyclyl ring (also referred to as a 4,6-membered heterocyclyl ring) include, without limitation, diazaspirononanyl (e.g., 2,7-diazaspiro[3.5]nonanyl). Exemplary 6-membered heterocyclyl groups fused to an aryl ring (also referred to herein as a 6,6-bicyclic heterocyclyl ring) include, without limitation, tetrahydroquinolinyl, tetrahydroisoquinolinyl, and the like. Exemplary 6-membered heterocyclyl groups fused to a cycloalkyl ring (also referred to herein as a 6,7-bicyclic heterocyclyl ring) include, without limitation, azabicyclooctanyl (e.g., (1,5)-8-azabicyclo[3.2.1]octanyl). Exemplary 6-membered heterocyclyl groups fused to a cycloalkyl ring (also referred to herein as a 6,8-bicyclic heterocyclyl ring) include, without limitation, azabicyclononanyl (e.g., 9-azabicyclo[3.3.1]nonanyl).

The terms “alkylene,” “alkenylene,” “alkynylene,” “haloalkylene,” “heteroalkylene,” “cycloalkylene,” or “heterocyclylene,” alone or as part of another substituent, mean, unless otherwise stated, a divalent radical derived from an alkyl, alkenyl, alkynyl, haloalkylene, heteroalkylene, cycloalkyl, or heterocyclyl respectively. For example, the term “alkenylene,” by itself or as part of another substituent, means, unless otherwise stated, a divalent radical derived from an alkene. An alkylene, alkenylene, alkynylene, haloalkylene, heteroalkylene, cycloalkylene, or heterocyclylene group may be described as, e.g., a C₁-C₆-membered alkylene, C₂-C₆-membered alkenylene, C₂-C₆-membered alkynylene, C₁-C₆-membered haloalkylene, C₁-C₆-membered heteroalkylene, C₃-C₈-membered cycloalkylene, or C₃-C₈-membered heterocyclylene, wherein the term “membered” refers to the non-hydrogen atoms within the moiety. In the case of heteroalkylene and heterocyclylene groups, heteroatoms can also occupy either or both of the chain termini (e.g., alkyleneoxy, alkylenedioxy, alkyleneamino, alkylenediamino, and the like). Still further, no orientation of the linking group is implied by the direction in which the formula of the linking group is written. For example, the formula -C(O)₂R¹— may represent both —C(O)₂R¹— and —R¹C(O)₂—.

As used herein, the terms “cyano” or “—CN” refer to a substituent having a carbon atom joined to a nitrogen atom by a triple bond, e.g., C≡N.

As used herein, the terms “halogen” or “halo” refer to fluorine, chlorine, bromine or iodine.

As used herein, the term “hydroxy” refers to —OH.

As used herein, the term “nitro” refers to a substitutent having two oxygen atoms bound to a nitrogen atom, e.g., —NO₂.

As used herein, the term “nucleobase” as used herein, is a nitrogen-containing biological compounds found linked to a sugar within a nucleoside—the basic building blocks of deoxyribonucleic acid (DNA) and ribonucleic acid (RNA). The primary, or naturally occurring, nucleobases are cytosine (DNA and RNA), guanine (DNA and RNA), adenine (DNA and RNA), thymine (DNA) and uracil (RNA), abbreviated as C, G, A, T, and U, respectively. Because A, G, C, and T appear in the DNA, these molecules are called DNA-bases; A, G, C, and U are called RNA-bases. Adenine and guanine belong to the double-ringed class of molecules called purines (abbreviated as R). Cytosine, thymine, and uracil are all pyrimidines. Other nucleobases that do not function as normal parts of the genetic code, are termed non-naturally occurring. In an embodiment, a nucleobase may be chemically modified, for example, with an alkyl (e.g., methyl), halo, -O-alkyl, or other modification.

As used herein, the term “nucleic acid” refers to deoxyribonucleic acids (DNA) or ribonucleic acids (RNA) and polymers thereof in either single- or double-stranded form. The term “nucleic acid” includes a gene, cDNA, pre-mRNA, or an mRNA. In one embodiment, the nucleic acid molecule is synthetic (e.g., chemically synthesized) or recombinant. Unless specifically limited, the term encompasses nucleic acids containing analogues or derivatives of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementarity sequences as well as the sequence explicitly indicated.

As used herein, “oxo” refers to a carbonyl, i.e., —C(O)—.

The symbol “ [symbol] ” as used herein in relation to a compound of Formula (I) refers to an attachment point to another moiety or functional group within the compound.

Alkyl, alkenyl, alkynyl, haloalkyl, heteroalkyl, cycloalkyl, heterocyclyl, aryl, and heteroaryl groups, as defined herein, are optionally substituted. In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, such as any of the substituents described herein that result in the formation of a stable compound. The present disclosure contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety.

Two or more substituents may optionally be joined to form aryl, heteroaryl, cycloalkyl, or heterocyclyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

The compounds provided herein may exist in one or more particular geometric, optical, enantiomeric, diasteriomeric, epimeric, stereoisomeric, tautomeric, conformational, or anomeric forms, including but not limited to: cis- and trans-forms; E- and Z-forms; endo- and exo-forms; R-, S-, and meso-forms; D- and L-forms; d- and 1-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and half chair-forms; and combinations thereof, hereinafter collectively referred to as “isomers” (or “isomeric forms”).

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. In an embodiment, the stereochemistry depicted in a compound is relative rather than absolute. Isomers can be isolated from mixtures by methods known to those skilled in the art, including chiral high-pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E.L. Eliel, Ed., Univ. of Notre Dame Press, Notre Dame, Ind. 1972). This disclosure additionally encompasses compounds described herein as individual isomers substantially free of other isomers, and alternatively, as mixtures of various isomers.

As used herein, a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 99% by weight, more than 99.5% by weight, or more than 99.9% by weight, of the enantiomer. In certain embodiments, the weights are based upon total weight of all enantiomers or stereoisomers of the compound.

In the compositions provided herein, an enantiomerically pure compound can be present with other active or inactive ingredients. For example, a pharmaceutical composition comprising an enantiomerically pure R—compound can comprise, for example, about 90% excipient and about 10% enantiomerically pure R—compound. In certain embodiments, the enantiomerically pure R—compound in such compositions can, for example, comprise, at least about 95% by weight R—compound and at most about 5% by weight S—compound, by total weight of the compound. For example, a pharmaceutical composition comprising an enantiomerically pure S— compound can comprise, for example, about 90% excipient and about 10% enantiomerically pure S—compound. In certain embodiments, the enantiomerically pure S—compound in such compositions can, for example, comprise, at least about 95% by weight S—compound and at most about 5% by weight R—compound, by total weight of the compound.

In some embodiments, a diastereomerically pure compound can be present with other active or inactive ingredients. For example, a pharmaceutical composition comprising a diastereometerically pure exo compound can comprise, for example, about 90% excipient and about 10% diastereometerically pure exo compound. In certain embodiments, the diastereometerically pure exo compound in such compositions can, for example, comprise, at least about 95% by weight exo compound and at most about 5% by weight endo compound, by total weight of the compound. For example, a pharmaceutical composition comprising a diastereometerically pure endo compound can comprise, for example, about 90% excipient and about 10% diastereometerically pure endo compound. In certain embodiments, the diastereometerically pure endo compound in such compositions can, for example, comprise, at least about 95% by weight endo compound and at most about 5% by weight exo compound, by total weight of the compound.

In some embodiments, an isomerically pure compound can be present with other active or inactive ingredients. For example, a pharmaceutical composition comprising a isomerically pure exo compound can comprise, for example, about 90% excipient and about 10% isomerically pure exo compound. In certain embodiments, the isomerically pure exo compound in such compositions can, for example, comprise, at least about 95% by weight exo compound and at most about 5% by weight endo compound, by total weight of the compound. For example, a pharmaceutical composition comprising an isomerically pure endo compound can comprise, for example, about 90% excipient and about 10% isomerically pure endo compound. In certain embodiments, the isomerically pure endo compound in such compositions can, for example, comprise, at least about 95% by weight endo compound and at most about 5% by weight exo compound, by total weight of the compound.

In certain embodiments, the active ingredient can be formulated with little or no excipient or carrier.

Compound described herein may also comprise one or more isotopic substitutions. For example, H may be in any isotopic form, including ¹H, ²H (D or deuterium), and ³H (T or tritium); C may be in any isotopic form, including ¹²C, ¹³C, and ¹⁴C; O may be in any isotopic form, including ¹⁶O and ¹⁸O; N may be in any isotopic form, including ¹⁴N and ¹⁵N; F may be in any isotopic form, including ¹⁸F, ¹⁹F, and the like.

The term “pharmaceutically acceptable salt” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al, Journal of Pharmaceutical Science 66: 1-19 (1977)). Certain specific compounds of the present invention contain both basic and acidic functionalities that allow the compounds to be converted into either base or acid addition salts. These salts may be prepared by methods known to those skilled in the art. Other pharmaceutically acceptable carriers known to those of skill in the art are suitable for the present invention.

In addition to salt forms, the present disclosure provides compounds in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

The term “solvate” refers to forms of the compound that are associated with a solvent, usually by a solvolysis reaction. This physical association may include hydrogen bonding. Conventional solvents include water, methanol, ethanol, acetic acid, DMSO, THF, diethyl ether, and the like. The compounds of Formulas (I), (III), or (V) may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolable solvates. Representative solvates include hydrates, ethanolates, and methanolates.

The term “hydrate” refers to a compound which is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound and wherein x is a number greater than 0. A given compound may form more than one type of hydrates, including, e.g., monohydrates (x is 1), lower hydrates (x is a number greater than 0 and smaller than 1, e.g., hemihydrates (R.0.5 H₂O)), and polyhydrates (x is a number greater than 1, e.g., dihydrates (R.2 H₂O) and hexahydrates (R.6 H₂O)).

The term “tautomer” refers to compounds that are interchangeable forms of a particular compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base. Another example of tautomerism is the aci- and nitro-forms of phenylnitromethane that are likewise formed by treatment with acid or base. Tautomeric forms may be relevant to the attainment of the optimal chemical reactivity and biological activity of a compound of interest.

### Other Definitions

The following definitions are more general terms used throughout the present disclosure.

The articles “a” and “an” refer to one or more than one (e.g., to at least one) of the grammatical object of the article. By way of example, “an element” means one element or more than one element. The term “and/or” means either “and” or “or” unless indicated otherwise.

The term “about” is used herein to mean within the typical ranges of tolerances in the art. For example, “about” can be understood as about 2 standard deviations from the mean. In certain embodiments, about means ±10%. In certain embodiments, about means +5%. When about is present before a series of numbers or a range, it is understood that “about” can modify each of the numbers in the series or range.

“Acquire” or “acquiring” as used herein, refer to obtaining possession of a value, e.g., a numerical value, or image, or a physical entity (e.g., a sample), by “directly acquiring” or “indirectly acquiring” the value or physical entity. “Directly acquiring” means performing a process (e.g., performing an analytical method or protocol) to obtain the value or physical entity. “Indirectly acquiring” refers to receiving the value or physical entity from another party or source (e.g., a third-party laboratory that directly acquired the physical entity or value). Directly acquiring a value or physical entity includes performing a process that includes a physical change in a physical substance or the use of a machine or device. Examples of directly acquiring a value include obtaining a sample from a human subject. Directly acquiring a value includes performing a process that uses a machine or device, e.g., mass spectrometer to acquire mass spectrometry data.

The terms “administer,” “administering,” or “administration,” as used herein refers to implanting, absorbing, ingesting, injecting, inhaling, or otherwise introducing an inventive compound, or a pharmaceutical composition thereof.

As used herein, the terms “condition,” “disease,” and “disorder” are used interchangeably.

An “effective amount” of a compound of Formulas (I), (III), or (V) refers to an amount sufficient to elicit the desired biological response, i.e., treating the condition. As will be appreciated by those of ordinary skill in this art, the effective amount of a compound of Formulas (I), (III), or (V) may vary depending on such factors as the desired biological endpoint, the pharmacokinetics of the compound, the condition being treated, the mode of administration, and the age and health of the subject. An effective amount encompasses therapeutic and prophylactic treatment. For example, in treating cancer, an effective amount of an inventive compound may reduce the tumor burden or stop the growth or spread of a tumor.

A “therapeutically effective amount” of a compound of Formulas (I), (III), or (V) is an amount sufficient to provide a therapeutic benefit in the treatment of a condition or to delay or minimize one or more symptoms associated with the condition. In some embodiments, a therapeutically effective amount is an amount sufficient to provide a therapeutic benefit in the treatment of a condition or to minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms or causes of the condition, or enhances the therapeutic efficacy of another therapeutic agent.

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprised therein. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types.

“Prevention,” “prevent,” and “preventing” as used herein refers to a treatment that comprises administering a therapy, e.g., administering a compound described herein (e.g., a compound of Formulas (I), (III), or (V)) prior to the onset of a disease, disorder, or condition in order to preclude the physical manifestation of said disease, disorder, or condition. In some embodiments, “prevention,” “prevent,” and “preventing” require that signs or symptoms of the disease, disorder, or condition have not yet developed or have not yet been observed. In some embodiments, treatment comprises prevention and in other embodiments it does not.

A “subject” to which administration is contemplated includes, but is not limited to, humans (i.e., a male or female of any age group, e.g., a pediatric subject (e.g., infant, child, adolescent) or adult subject (e.g., young adult, middle—aged adult, or senior adult)) and/or other non-human animals, for example, mammals (e.g., primates (e.g., cynomolgus monkeys, rhesus monkeys); commercially relevant mammals such as cattle, pigs, horses, sheep, goats, cats, and/or dogs) and birds (e.g., commercially relevant birds such as chickens, ducks, geese, and/or turkeys). In certain embodiments, the animal is a mammal. The animal may be a male or female and at any stage of development. A non-human animal may be a transgenic animal.

As used herein, the terms “treatment,” “treat,” and “treating” refer to reversing, alleviating, delaying the onset of, or inhibiting the progress of one or more of a symptom, manifestation, or underlying cause of a disease, disorder, or condition (e.g., as described herein), e.g., by administering a therapy, e.g., administering a compound described herein (e.g., a compound of Formulas (I), (III), or (V)). In an embodiment, treating comprises reducing, reversing, alleviating, delaying the onset of, or inhibiting the progress of a symptom of a disease, disorder, or condition. In an embodiment, treating comprises reducing, reversing, alleviating, delaying the onset of, or inhibiting the progress of a manifestation of a disease, disorder, or condition. In an embodiment, treating comprises reducing, reversing, alleviating, reducing, or delaying the onset of, an underlying cause of a disease, disorder, or condition. In some embodiments, “treatment,” “treat,” and “treating” require that signs or symptoms of the disease, disorder, or condition have developed or have been observed. In other embodiments, treatment may be administered in the absence of signs or symptoms of the disease or condition, e.g., in preventive treatment. For example, treatment may be administered to a susceptible individual prior to the onset of symptoms (e.g., in light of a history of symptoms and/or in light of genetic or other susceptibility factors). Treatment may also be continued after symptoms have resolved, for example, to delay or prevent recurrence. Treatment may also be continued after symptoms have resolved, for example, to delay or prevent recurrence. In some embodiments, treatment comprises prevention and in other embodiments it does not.

A “proliferative disease” refers to a disease that occurs due to abnormal extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); 4) the pathological angiogenesis as in proliferative retinopathy and tumor metastasis; or 5) evasion of host immune surveillance and elimination of neoplastic cells. Exemplary proliferative diseases include cancers (i.e., “malignant neoplasms”), benign neoplasms, and angiogenesis.

A “non-proliferative disease” refers to a disease that does not primarily extend through the abnormal multiplication of cells. A non-proliferative disease may be associated with any cell type or tissue type in a subject. Exemplary non-proliferative diseases include neurological diseases or disorders (e.g., a repeat expansion disease); autoimmune disease or disorders; immunodeficiency diseases or disorders; lysosomal storage diseases or disorders; inflammatory diseases or disorders; cardiovascular conditions, diseases, or disorders; metabolic diseases or disorders; respiratory conditions, diseases, or disorders; renal diseases or disorders; and infectious diseases.

### Compounds

In one aspect, the present disclosure features a compound of Formula (I):

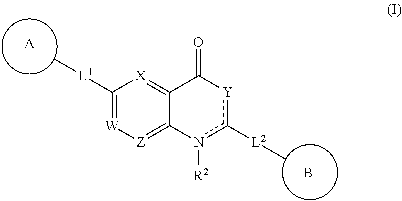

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; each of L¹ and L² is independently is absent, C₁-C₆-alkylene, C₁-C₆-heteroalkylene, —O—, —C(O)—, —N(R⁸)—, —N(R⁸)C(O)—, or —C(O)N(R⁸)—, wherein each alkylene and heteroalkylene is optionally substituted with one or more R⁹; each of W, X, and Z is independently C(R³) or N; Y is N, N(R^{4a}), C(R^{4b}), or C(R^{4b})(R^{4c}), wherein the dashed lines in the ring comprising Y may be single or double bonds as valency permits; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁵; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵; R² is absent, hydrogen, or C₁-C₆-alkyl; R³ is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —C(O)R^{D}, or —C(O)OR^{D}; R^{4a} is hydrogen, C₁-C₆-alkyl, C₁-C₆-heteroalkyl, or C₁-C₆-haloalkyl; each of R^{4b} and R^{4c} is independently hydrogen, C₁-C₆-alkyl, C₁-C₆-heteroalkyl, C₁-C₆-haloalkyl, halo, or —OR^{A}; each R⁵ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁶; each R⁶ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R⁸ is independently hydrogen, C₁-C₆-alkyl, or C₁-C₆-haloalkyl; each R⁹ is independently C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each R^{B} and R^{C} is independently hydrogen, C₁-C₆ alkyl, C₁-C₆ heteroalkyl, cycloalkyl, heterocyclyl, —OR^{A}; or R^{B} and R^{C} together with the atom to which they are attached form a 3-7-membered heterocyclyl ring optionally substituted with one or more R¹⁰; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R¹⁰ is independently C₁-C₆-alkyl or halo; and x is 0, 1, or 2.

In another aspect, the present disclosure features a compound of Formula (III):

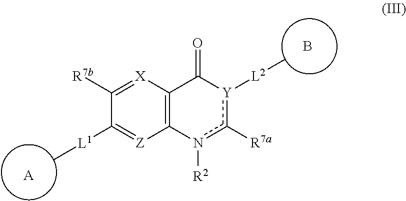

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; each of L¹ and L² is independently absent, C₁-C₆-alkylene, C₁-C₆-heteroalkylene, —O—, —C(O)—, —N(R⁸)—, —N(R⁸)C(O)—, or —C(O)N(R⁸)—, wherein each alkylene and heteroalkylene is optionally substituted with one or more R⁹; each of X and Z is independently C(R³) or N; Y is N, C, or C(R^{4b}), wherein the dashed lines in the ring comprising Y may be single or double bonds as valency permits; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁵; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵; R² is absent, hydrogen, or C₁-C₆-alkyl; R³ is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —C(O)R^{D}, or —C(O)OR^{D}; R^{4b} is hydrogen, C₁-C₆-alkyl, C₁-C₆-heteroalkyl, or C₁-C₆-haloalkyl; each R⁵ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁶; each R⁶ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; R^{7a} is hydrogen, C₁-C₆-alkyl, C₁-C₆-heteroalkyl, C₁-C₆-haloalkyl, halo, cyano, oxo, or —OR^{A}; R^{7b}is hydrogen, C₁-C₆-alkyl, C₁-C₆-heteroalkyl, C₁-C₆-haloalkyl, halo, cyano, or —OR^{A}; each R⁸ is independently hydrogen, C₁-C₆-alkyl, or C₁-C₆-haloalkyl; each R⁹ is independently C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each R^{B} and R^{C} is independently hydrogen, C₁-C₆ alkyl, C₁-C₆ heteroalkyl, cycloalkyl, heterocyclyl, —OR^{A}; or R^{B} and R^{C} together with the atom to which they are attached form a 3-7-membered heterocyclyl ring optionally substituted with one or more R¹⁰; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R¹⁰ is independently C₁-C₆-alkyl or halo; and x is 0, 1, or 2.

In another aspect, the present disclosure features a compound of Formula (V):

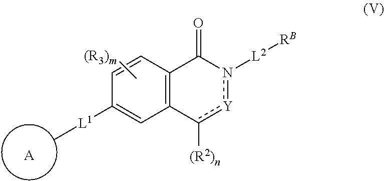

or a pharmaceutically acceptable salt, solvate, hydrate, tautomer, or stereoisomer thereof, wherein A is cycloalkyl, heterocyclyl, aryl, or heteroaryl, each of which is optionally substituted with one or more R¹; R^{B} is B, C₁-C₆-alkyl, or C₁-C₆-heteroalkyl, wherein alkyl and heteroalkyl are substituted by one or more R¹⁰; B is cycloalkyl, heterocyclyl, aryl, or heteroaryl, each of which is optionally substituted with one or more R¹; each of which is optionally substituted with one or more R¹; each of L¹ and L² is independently absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁹; Y is N, C(R^{6a}), or C(R^{6a})(R^{6b}), wherein the dashed lines in the ring comprising Y may be single or double bonds as valency permits; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁵; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵; each R² is independently hydrogen or C₁-C₆-alkyl; R³ is C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —C(O)R^{D}, or —C(O)OR^{D}; R⁴ is hydrogen, C₁-C₆-alkyl, or C₁-C₆-haloalkyl; each R⁵ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁷; R^{6a} and R^{6b} is independently hydrogen, C₁-C₆-alkyl, C₁-C₆-heteroalkyl, Cᵢ-C₆-haloalkyl, or halo; each R⁷ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each R^{B} and R^{C} is independently hydrogen, C₁-C₆ alkyl, C₁-C₆ heteroalkyl, cycloalkyl, heterocyclyl, —OR^{A}; or R^{B} and R^{C} together with the atom to which they are attached form a 3-7-membered heterocyclyl ring optionally substituted with one or more R⁹; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁹ and R¹⁰ is independently C₁-C₆-alkyl or halo; n is 0, 1, or 2; m is 0, 1, 2, or 3; and x is 0, 1, or 2.

In some embodiments, for Formula (V), R^{B} is B, wherein B is cycloalkyl, heterocyclyl, aryl, or heteroaryl, each of which is optionally substituted with one or more R¹.

As generally described herein for compounds of Formula (I), (III), and (V), each of A or B are independently cycloalkyl, heterocyclyl, aryl, or heteroaryl, each of which is optionally substituted with one or more R¹.

In some embodiments, each of A and B are independently a monocyclic ring, e.g., monocyclic cycloalkyl, monocyclic heterocyclyl, monocyclic aryl, or monocyclic heteroaryl. The monocyclic ring may be saturated, partially unsaturated, or fully unsaturated (e.g., aromatic). In some embodiments, A or B are independently a monocyclic ring comprising between 3 and 10 ring atoms (e.g., 3, 4, 5, 6, 7, 8, 9, or 10 ring atoms). In some embodiments, A is a 4-membered monocyclic ring. In some embodiments, B is a 4-membered monocyclic ring. In some embodiments, A is a 5-membered monocyclic ring. In some embodiments, B is a 5-membered monocyclic ring. In some embodiments, A is a 6-membered monocyclic ring. In some embodiments, B is a 6-membered monocyclic ring. In some embodiments, A is a 7-membered monocyclic ring. In some embodiments, B is a 7-membered monocyclic ring. In some embodiments, A is an 8-membered monocyclic ring. In some embodiments, B is an 8-membered monocyclic ring. In some embodiments, A or B are independently a monocyclic ring optionally substituted with one or more R¹.

In some embodiments, A or B are independently a bicyclic ring, e.g., bicyclic cycloalkyl, bicyclic heterocyclyl, bicyclic aryl, or bicyclic heteroaryl. The bicyclic ring may be saturated, partially unsaturated, or fully unsaturated (e.g., aromatic). In some embodiments, A or B are independently a bicyclic ring comprising a fused, bridged, or spiro ring system. In some embodiments, A or B are independently a bicyclic ring comprising between 4 and 18 ring atoms (e.g., 4, 5, 6, 7, 8, 9, 10, 11, 12, 13, 14, 15, 16, 17, or 18 ring atoms). In some embodiments, A is a 6-membered bicyclic ring. In some embodiments, B is a 6-membered bicyclic ring. In some embodiments, A is a 7-membered bicyclic ring. In some embodiments, B is a 7-membered bicyclic ring. In some embodiments, A is an 8-membered bicyclic ring. In some embodiments, B is an 8-membered bicyclic ring. In some embodiments, A is a 9-membered bicyclic ring. In some embodiments, B is a 9-membered bicyclic ring. In some embodiments, A is a 10-membered bicyclic ring. In some embodiments, B is a 10-membered bicyclic ring. In some embodiments, A is an 11-membered bicyclic ring. In some embodiments, B is an 11-membered bicyclic ring. In some embodiments, A is a 12-membered bicyclic ring. In some embodiments, B is a 12-membered bicyclic ring. In some embodiments, A or B are independently a bicyclic ring optionally substituted with one or more R¹.

In some embodiments, A or B are independently a tricyclic ring, e.g., tricyclic cycloalkyl, tricyclic heterocyclyl, tricyclic aryl, or tricyclic heteroaryl. The tricyclic ring may be saturated, partially unsaturated, or fully unsaturated (e.g., aromatic). In some embodiments, A or B are independently a tricyclic ring that comprises a fused, bridged, or spiro ring system, or a combination thereof. In some embodiments, A or B are independently a tricyclic ring comprising between 6 and 24 ring atoms (e.g., 6, 7, 8, 9, 10, 11, 12, 13, 14, 15, 16, 17, 18, 19, 20, 21, 22, 23, or 24 ring atoms). In some embodiments, A is an 8-membered tricyclic ring. In some embodiments, B is an 8-membered tricyclic ring. In some embodiments, A is a 9-membered tricyclic ring. In some embodiments, B is a 9-membered tricyclic ring. In some embodiments, A is a 10-membered tricyclic ring. In some embodiments, B is a 10-membered tricyclic ring. In some embodiments, A or B are independently a tricyclic ring optionally substituted with one or more R¹.

In some embodiments, A or B are independently monocyclic cycloalkyl, monocyclic heterocyclyl, monocyclic aryl, or monocyclic heteroaryl. In some embodiments, A or B are independently bicyclic cycloalkyl, bicyclic heterocyclyl, bicyclic aryl, or bicyclic heteroaryl. In some embodiments, A or B are independently tricyclic cycloalkyl, tricyclic heterocyclyl, tricyclic aryl, or tricyclic heteroaryl. In some embodiments, A is monocyclic heterocyclyl. In some embodiments, B is monocyclic heterocyclyl. In some embodiments, A is bicyclic heterocyclyl. In some embodiments, B is bicyclic heterocyclyl. In some embodiments, A is monocyclic heteroaryl. In some embodiments, B is monocyclic heteroaryl. In some embodiments, A is bicyclic heteroaryl. In some embodiments, B is bicyclic heteroaryl.

In some embodiments, A or B are independently a nitrogen-containing heterocyclyl, e.g., heterocyclyl comprising one or more nitrogen atom. The one or more nitrogen atom of the nitrogen-containing heterocyclyl may be at any position of the ring. In some embodiments, the nitrogen-containing heterocyclyl is monocyclic, bicyclic, or tricyclic. In some embodiments, A or B are independently heterocyclyl comprising at least 1, at least 2, at least 3, at least 4, at least 5, or at least 6 nitrogen atoms. In some embodiments, A is heterocyclyl comprising 1 nitrogen atom. In some embodiments, B is heterocyclyl comprising 1 nitrogen atom. In some embodiments, A is heterocyclyl comprising 2 nitrogen atoms. In some embodiments, B is heterocyclyl comprising 2 nitrogen atoms. In some embodiments, A is heterocyclyl comprising 3 nitrogen atoms. In some embodiments, B is heterocyclyl comprising 3 nitrogen atoms. In some embodiments, A is heterocyclyl comprising 4 nitrogen atoms. In some embodiments, B is heterocyclyl comprising 4 nitrogen atoms. In some embodiments, A or B are independently a nitrogen-containing heterocyclyl comprising one or more additional heteroatoms, e.g., one or more of oxygen, sulfur, boron, silicon, or phosphorus. In some embodiments, the one or more nitrogen of the nitrogen-containing heterocyclyl is substituted, e.g., with R¹.

In some embodiments, A or B are independently a nitrogen-containing heteroaryl, e.g., heteroaryl comprising one or more nitrogen atom. The one or more nitrogen atom of the nitrogen-containing heteroaryl may be at any position of the ring. In some embodiments, the nitrogen-containing heteroaryl is monocyclic, bicyclic, or tricyclic. In some embodiments, A or B are independently heteroaryl comprising at least 1, at least 2, at least 3, at least 4, at least 5, or at least 6 nitrogen atoms. In some embodiments, A is heteroaryl comprising 1 nitrogen atom. In some embodiments, B is heteroaryl comprising 1 nitrogen atom. In some embodiments, A is heteroaryl comprising 2 nitrogen atoms. In some embodiments, B is heteroaryl comprising 2 nitrogen atoms. In some embodiments, A is heteroaryl comprising 3 nitrogen atoms. In some embodiments, B is heteroaryl comprising 3 nitrogen atoms. In some embodiments, A is heteroaryl comprising 4 nitrogen atoms. In some embodiments, B is heteroaryl comprising 4 nitrogen atoms. In some embodiments, A or B are independently a nitrogen-containing heteroaryl comprising one or more additional heteroatoms, e.g., one or more of oxygen, sulfur, boron, silicon, or phosphorus. In some embodiments, the one or more nitrogen of the nitrogen-containing heteroaryl is substituted, e.g., with R¹.

In some embodiments, A is a 6-membered nitrogen-containing heterocyclyl, e.g., a 6-membered heterocyclyl comprising one or more nitrogen. In some embodiments, A is a 6-membered heterocyclyl comprising 1 nitrogen atom. In some embodiments, A is a 6-membered heterocyclyl comprising 2 nitrogen atoms. In some embodiments, A is a 6-membered heterocyclyl comprising 3 nitrogen atoms. In some embodiments, A is a 6-membered heterocyclyl comprising 4 nitrogen atoms. The one or more nitrogen atom of the 6-membered nitrogen-containing heterocyclyl may be at any position of the ring. In some embodiments, A is a 6-membered nitrogen-containing heterocyclyl optionally substituted with one or more R¹. In some embodiments, the one or more nitrogen of the 6-membered nitrogen-containing heterocyclyl is substituted, e.g., with R¹. In some embodiments, A is a 6-membered nitrogen-containing heterocyclyl comprising one or more additional heteroatoms, e.g., one or more of oxygen, sulfur, boron, silicon, or phosphorus.

In some embodiments, B is a 5-membered nitrogen-containing heterocyclyl or heteroaryl, e.g., a 5-membered heterocyclyl or heteroaryl comprising one or more nitrogen. In some embodiments, B is a 5-membered heterocyclyl comprising 1 nitrogen atom. In some embodiments, B is a 5-membered heteroaryl comprising 1 nitrogen atom. In some embodiments, B is a 5-membered heterocyclyl comprising 2 nitrogen atoms. In some embodiments, B is a 5-membered heteroaryl comprising 2 nitrogen atoms. In some embodiments, B is a 5-membered heterocyclyl comprising 3 nitrogen atoms. In some embodiments, B is a 5-membered heteroaryl comprising 3 nitrogen atoms. The one or more nitrogen atom of the 5-membered nitrogen-containing heterocyclyl or heteroaryl may be at any position of the ring. In some embodiments, B is a 5-membered nitrogen-containing heterocyclyl optionally substituted with one or more R¹. In some embodiments, B is a 5-membered nitrogen-containing heteroaryl optionally substituted with one or more R¹. In some embodiments, the one or more nitrogen of the 5-membered nitrogen-containing heterocyclyl or heteroaryl is substituted, e.g., with R¹. In some embodiments, B is a 5-membered nitrogen-containing heterocyclyl or heteroaryl comprising one or more additional heteroatoms, e.g., one or more of oxygen, sulfur, boron, silicon, or phosphorus.

In some embodiments, B is a nitrogen-containing bicyclic heteroaryl (e.g., a 9-membered nitrogen-containing bicyclic heteroaryl), that is optionally substituted with one or more R¹. In some embodiments, B is a 9-membered bicyclic heteroaryl comprising 1 nitrogen atom. In some embodiments, B is a 9-membered bicyclic heteroaryl comprising 2 nitrogen atoms. In some embodiments, B is a 9-membered bicyclic heteroaryl comprising 3 nitrogen atoms. In some embodiments, B is a 9-membered bicyclic heteroaryl comprising 4 nitrogen atoms. The one or more nitrogen atom of the 9-membered bicyclic heteroaryl may be at any position of the ring. In some embodiments, B is a 9-membered bicyclic heteroaryl substituted with one or more R¹.

In some embodiments, each of A and B are independently selected from:

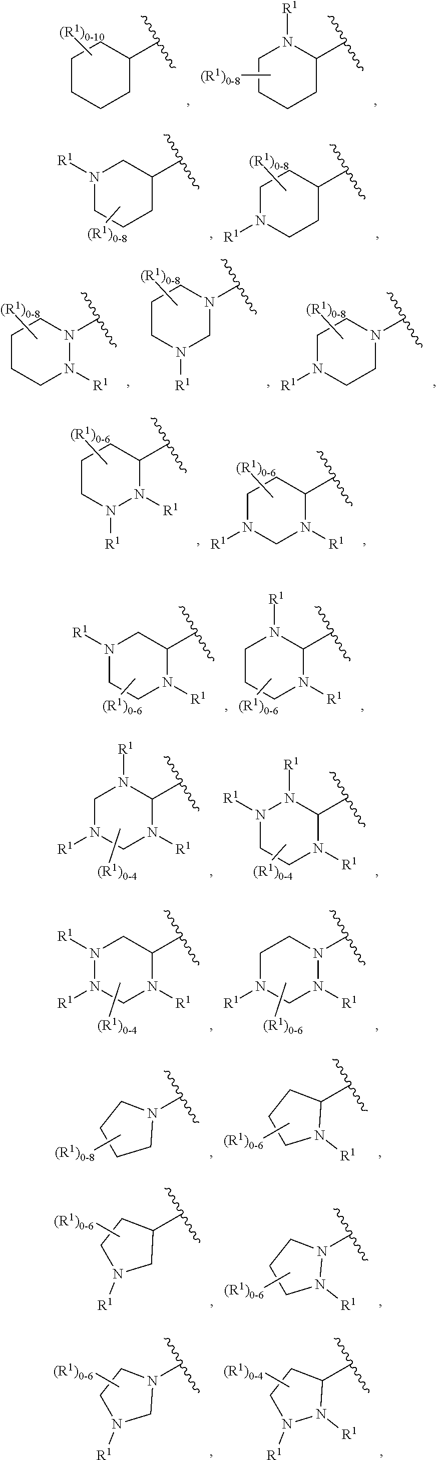

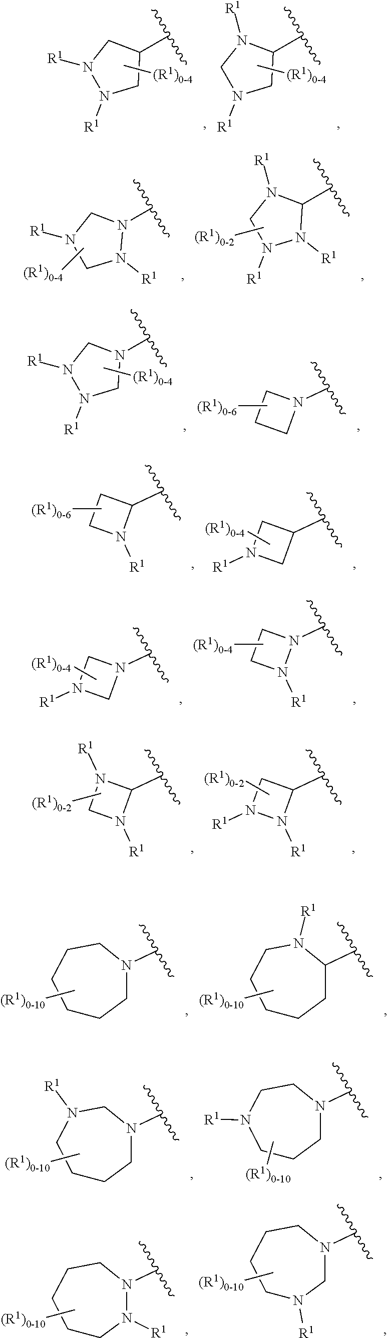

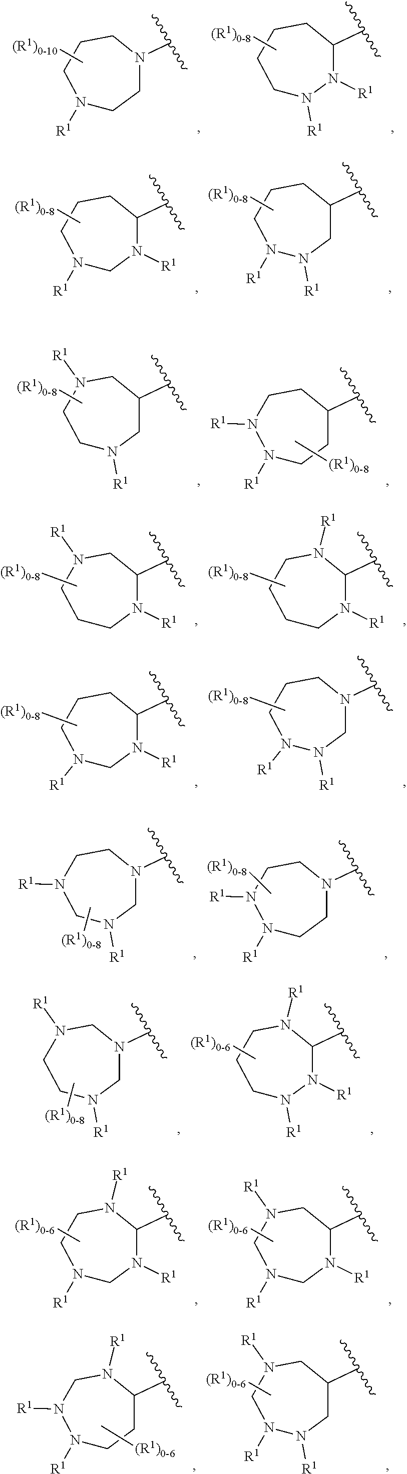

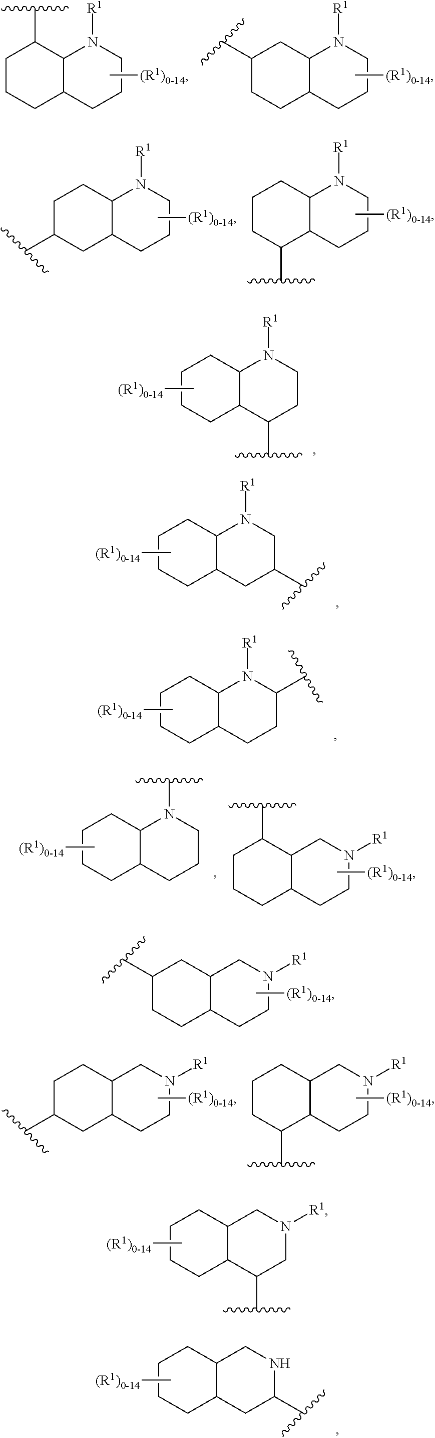

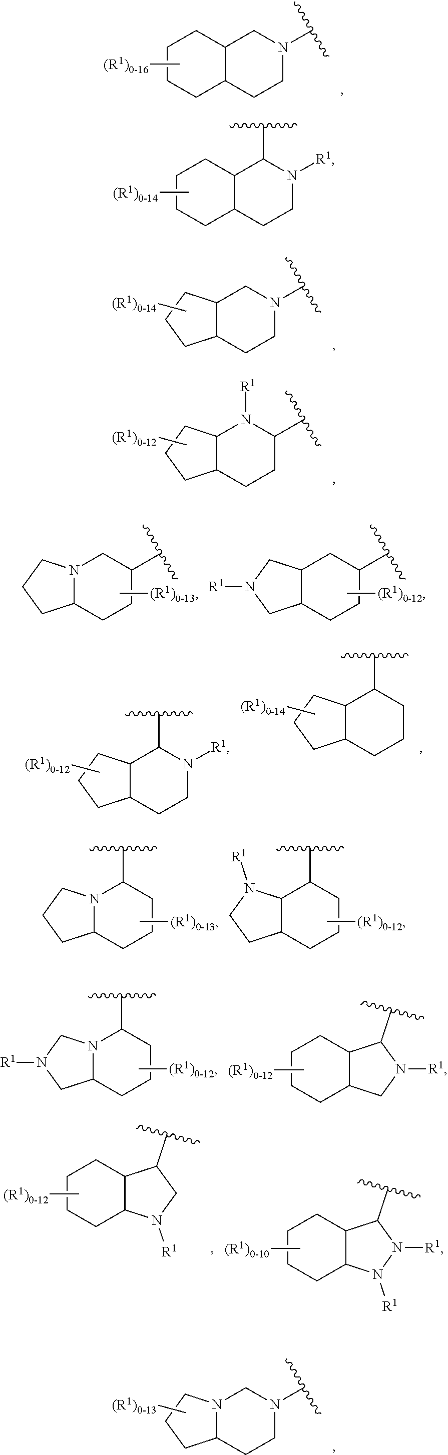

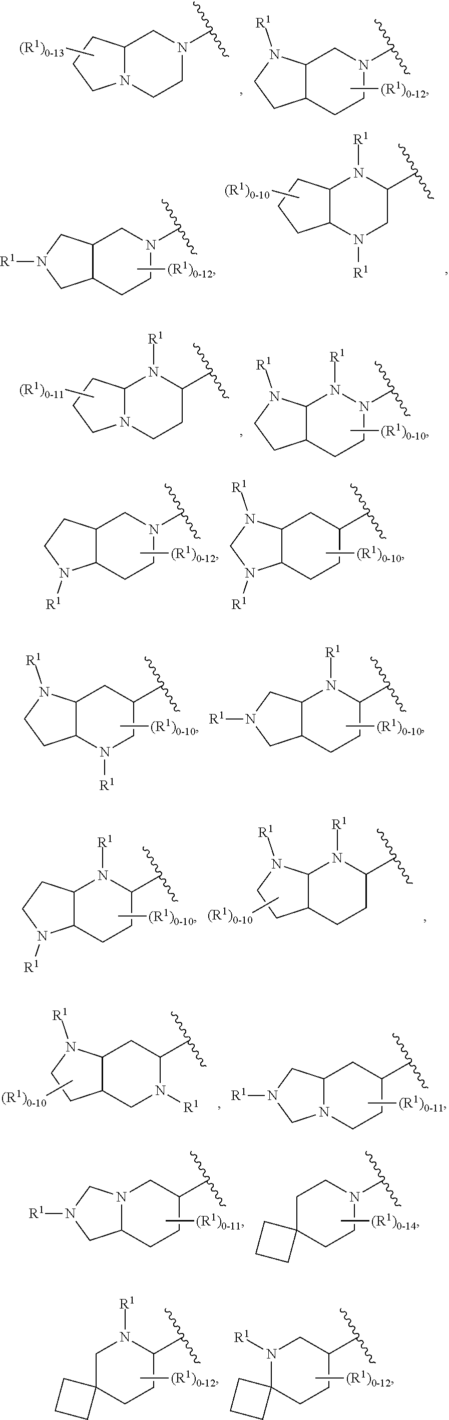

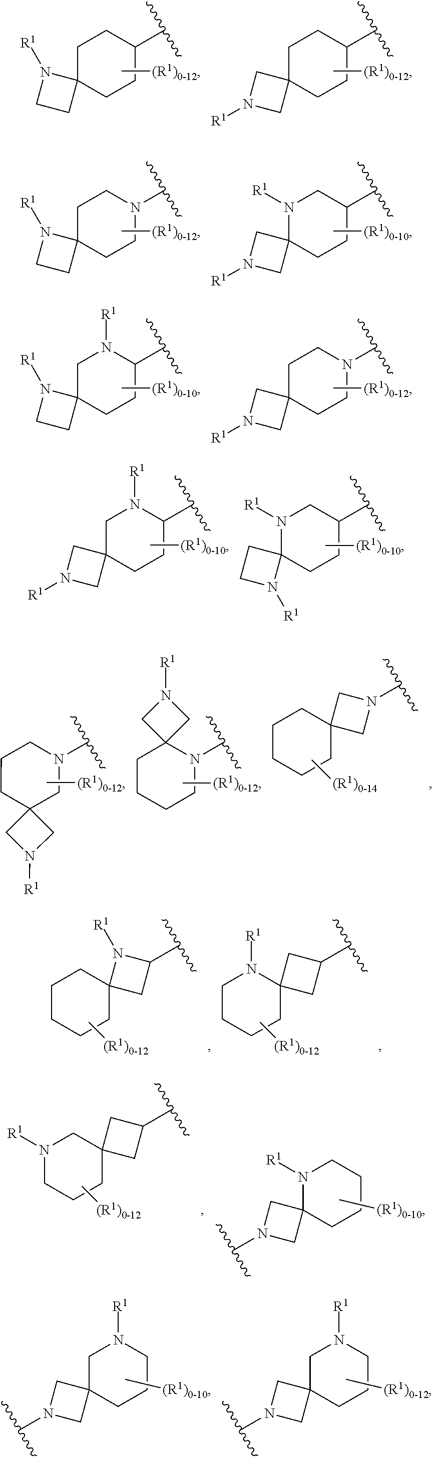

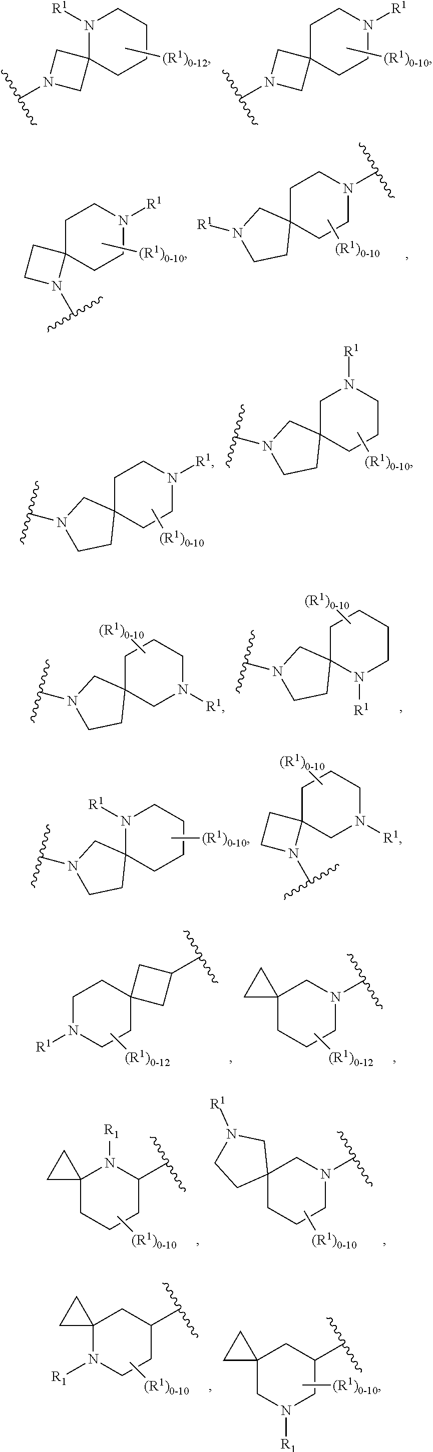

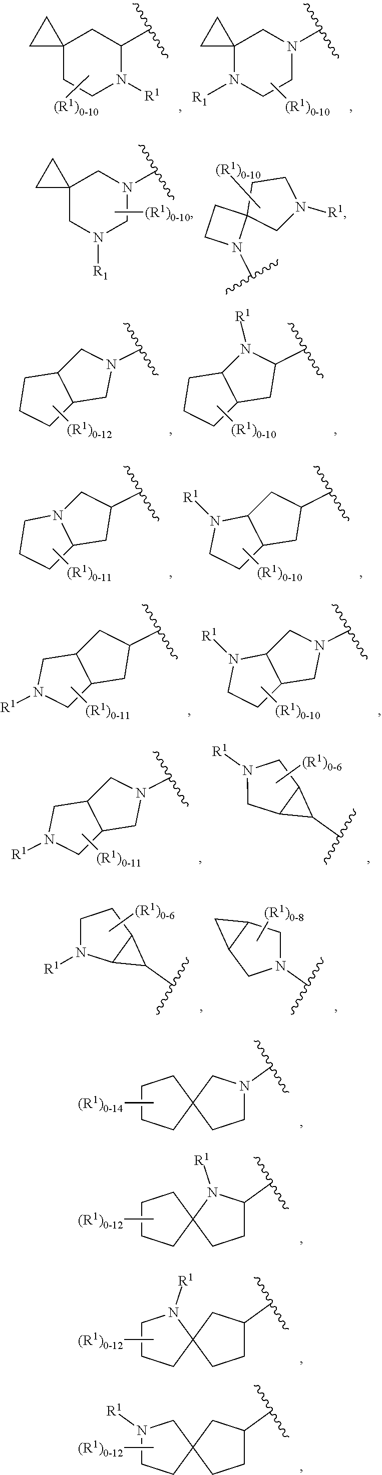

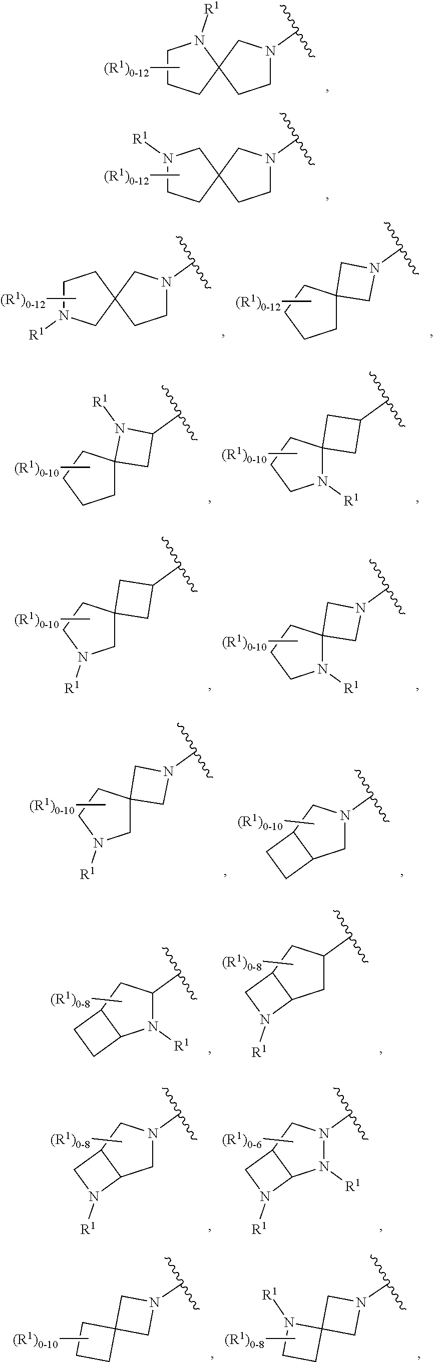

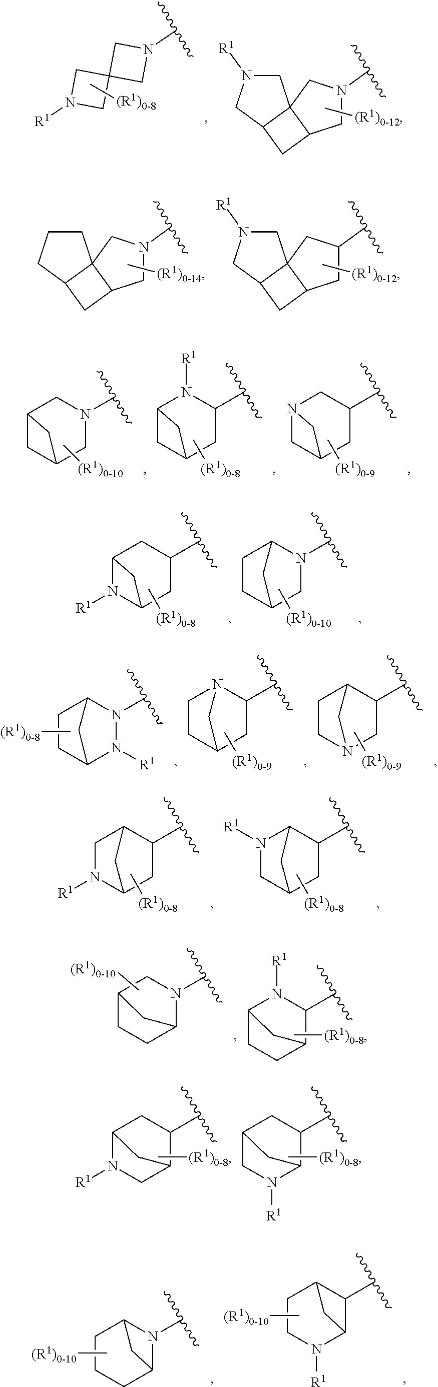

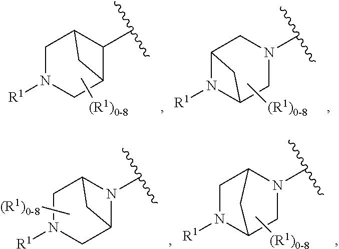

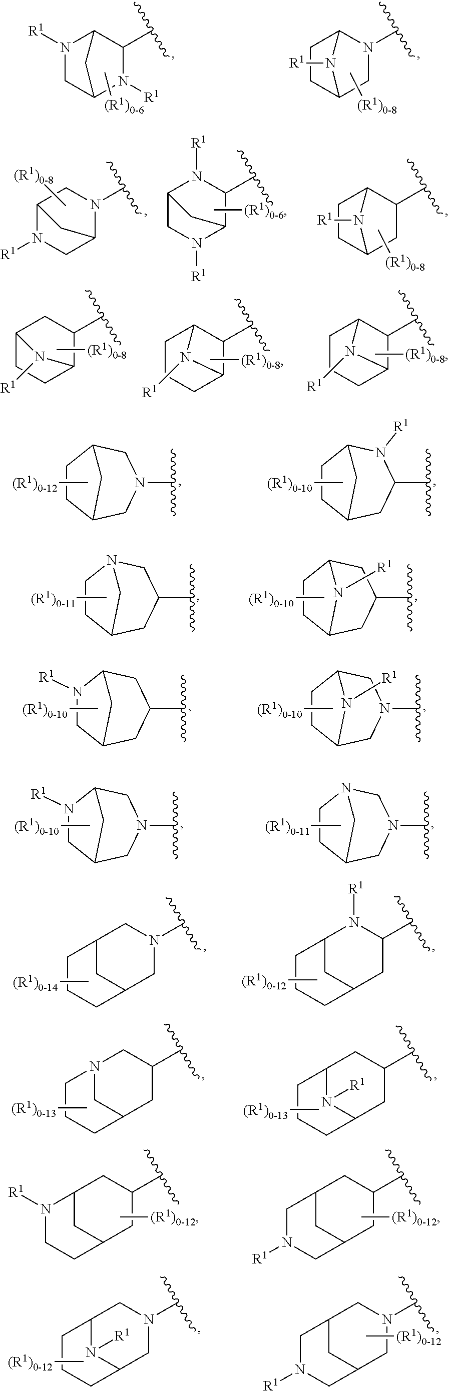

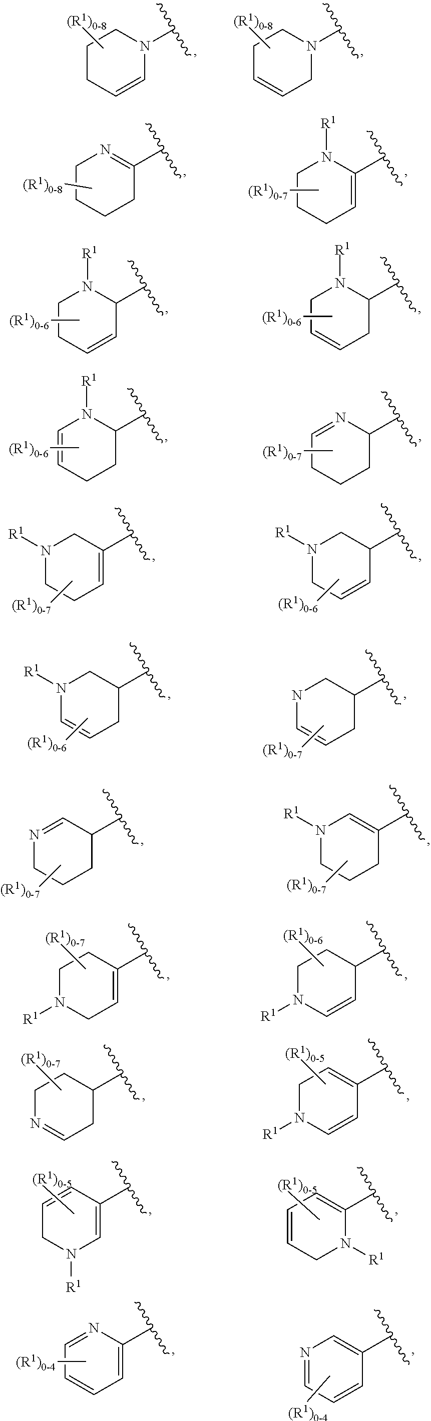

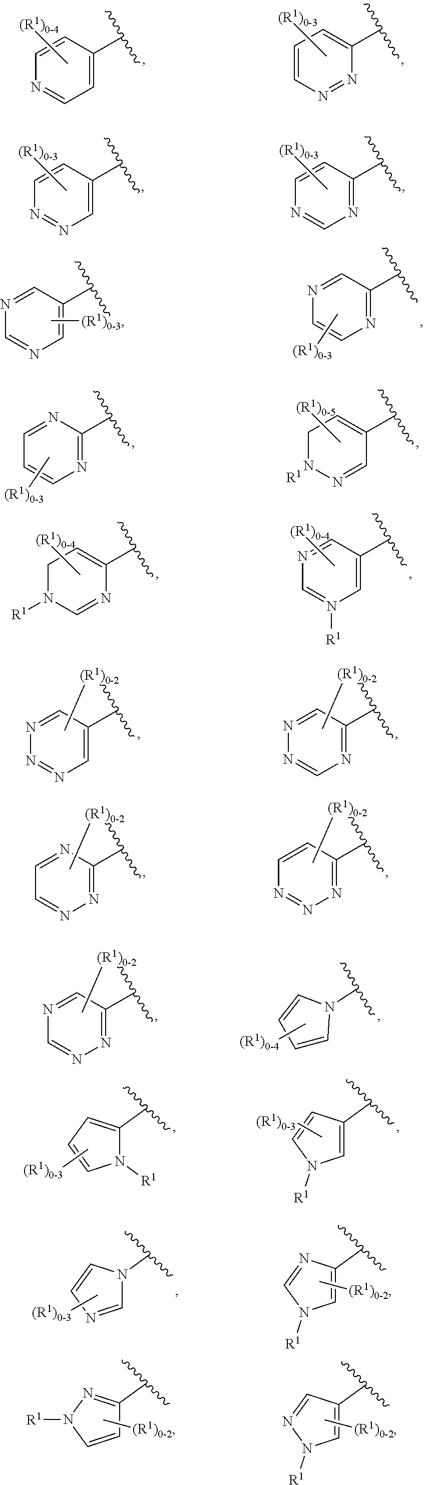

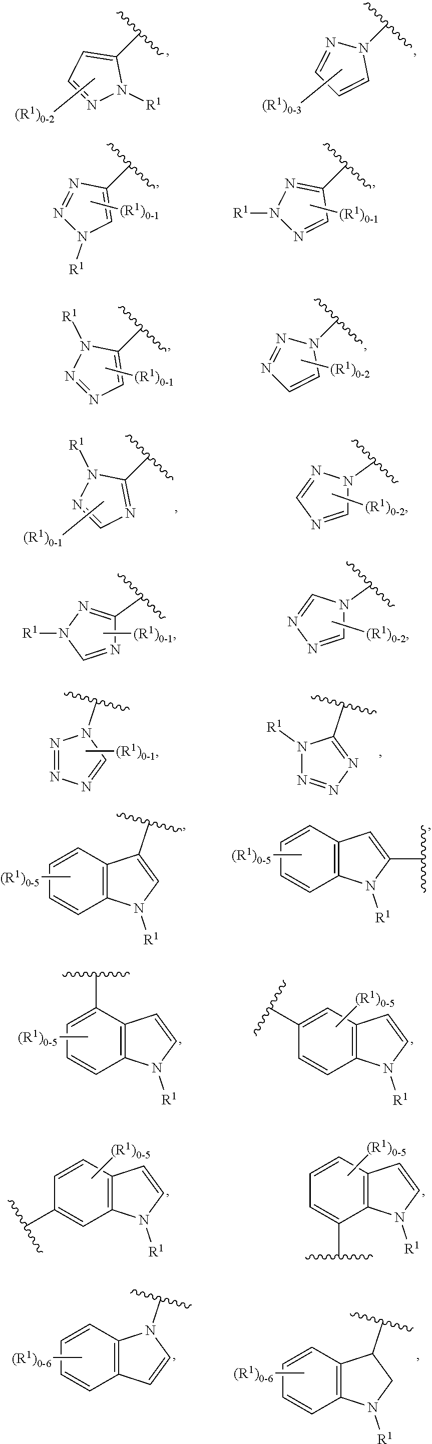

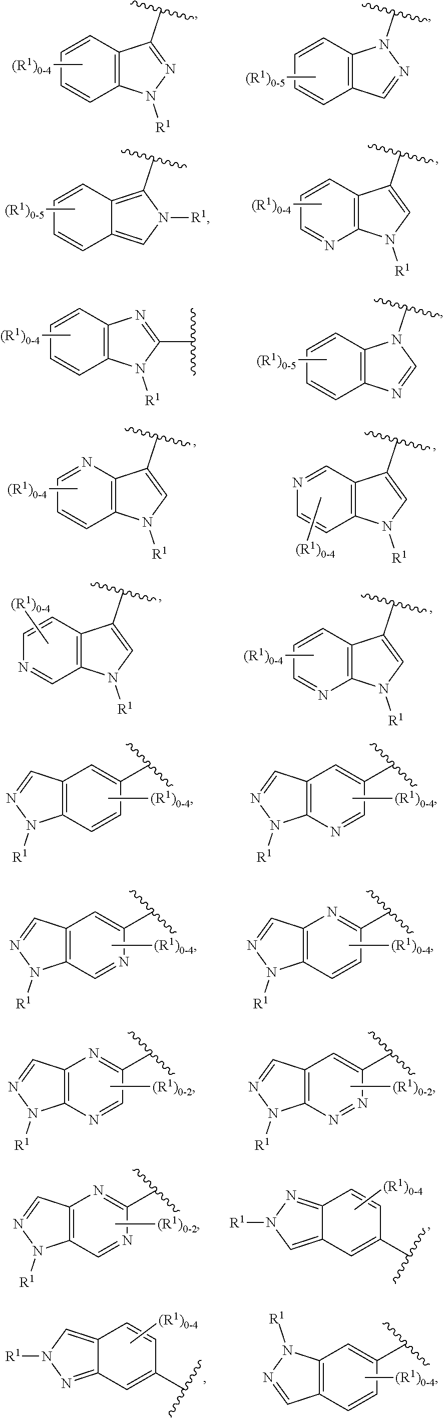

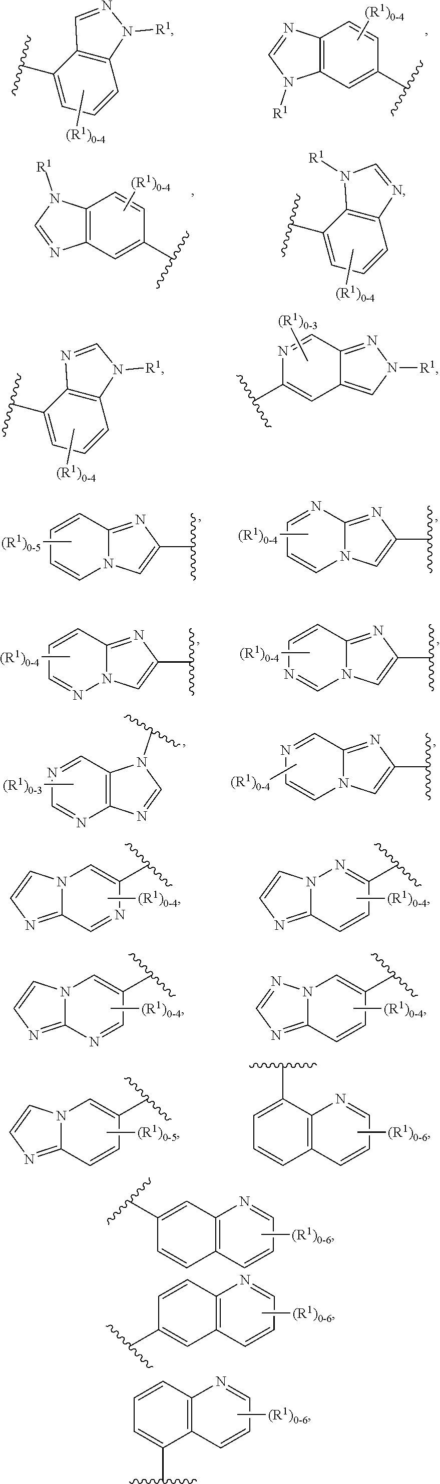

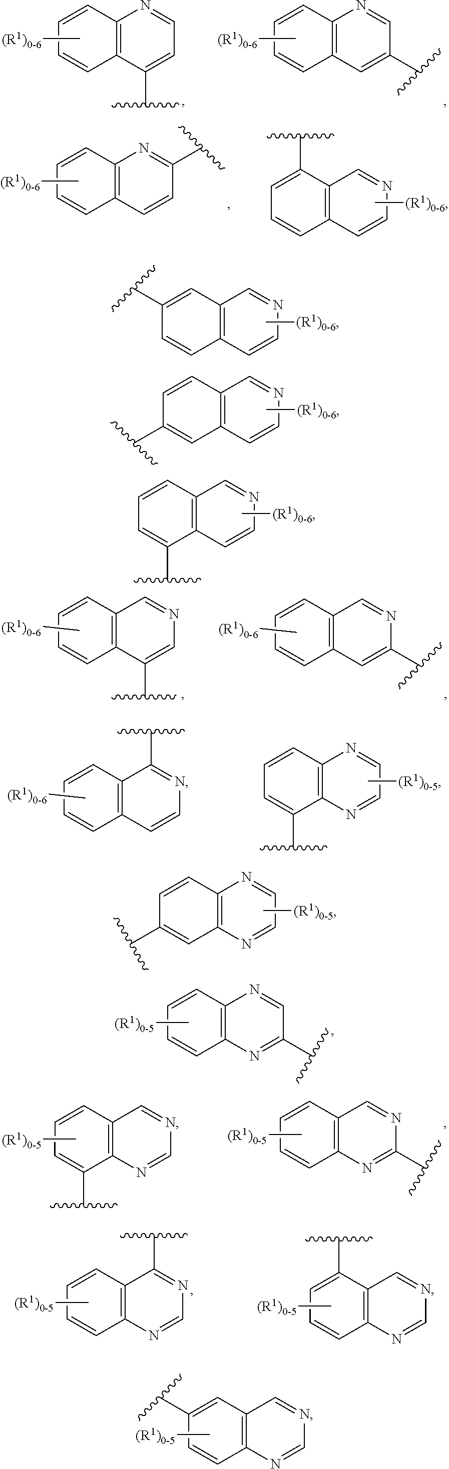

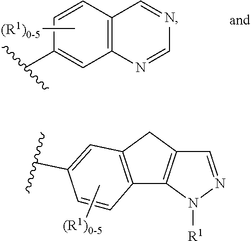

wherein each R¹ is as defined herein. In an embodiment, A and B are each independently a saturated, partially saturated, or unsaturated (e.g., aromatic) derivative of one of the rings described above. In an embodiment, A and B are each independently a stereoisomer of one of the rings described above.

In some embodiments, each of A and B are independently selected from:

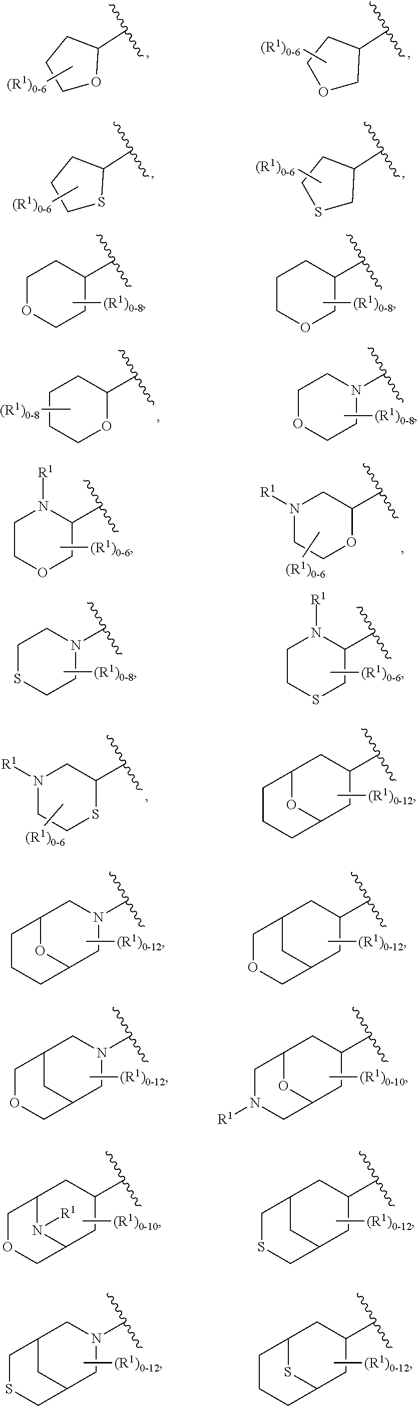

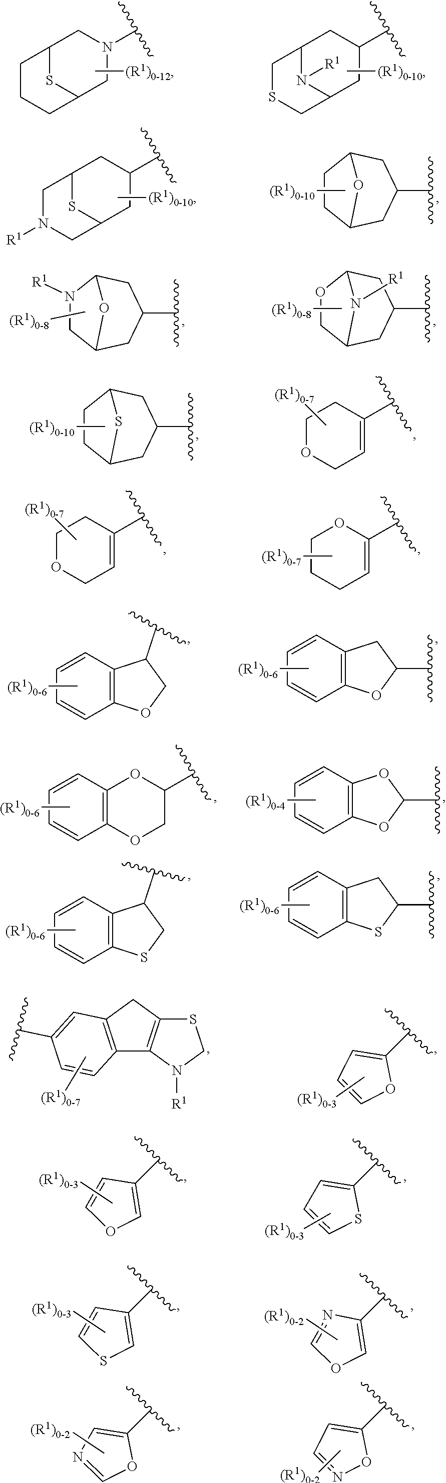

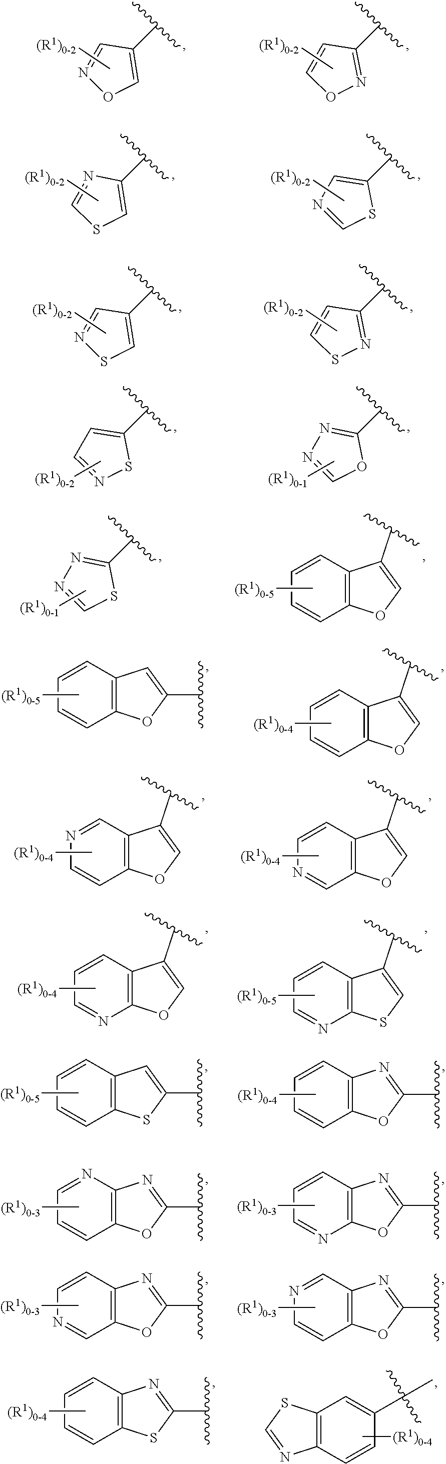

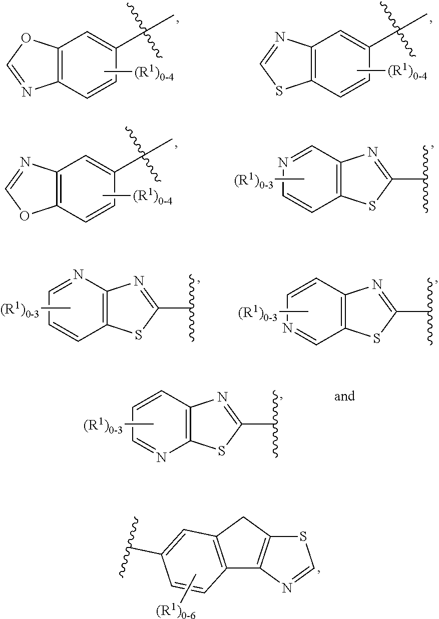

wherein each R¹ is as defined herein. In an embodiment, A and B are each independently a saturated, partially saturated, or unsaturated (e.g., aromatic) derivative of one of the rings described above. In an embodiment, A and B are each independently a stereoisomer of one of the rings described above.

In some embodiments, A is heterocyclyl. In some embodiments, A is a nitrogen-containing heterocyclyl. In some embodiments, A is a monocyclic nitrogen-containing heterocyclyl. In some embodiments, A is selected from

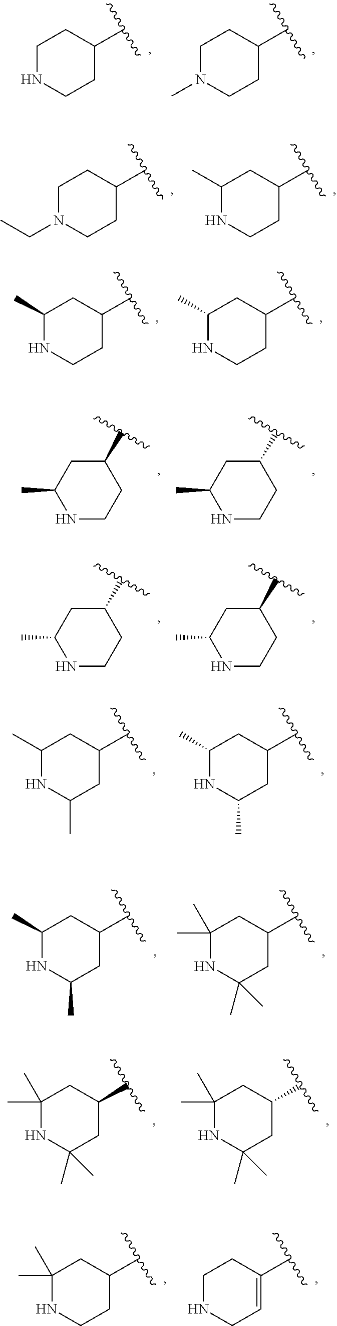

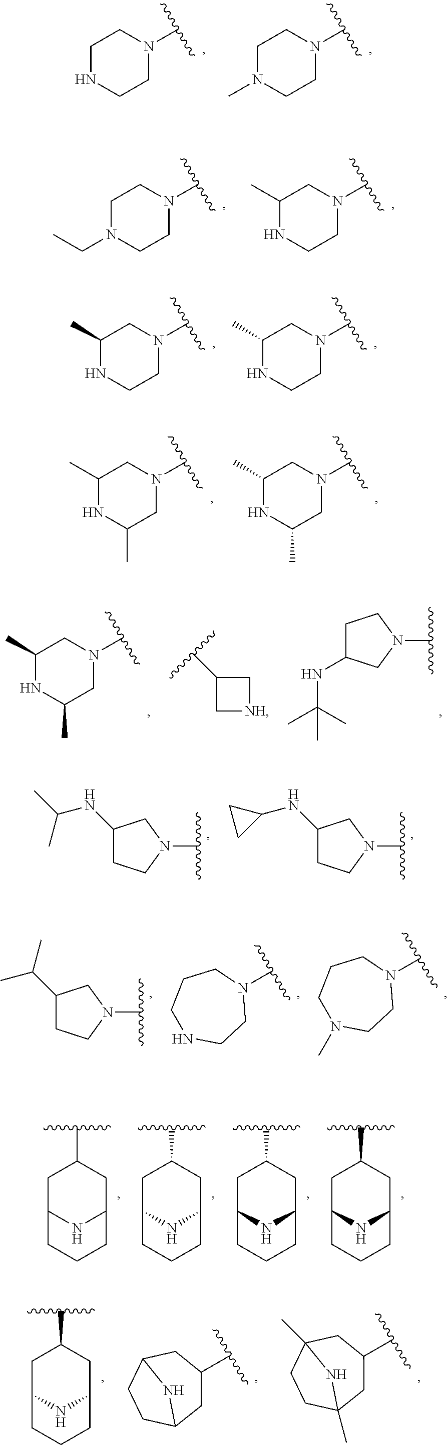

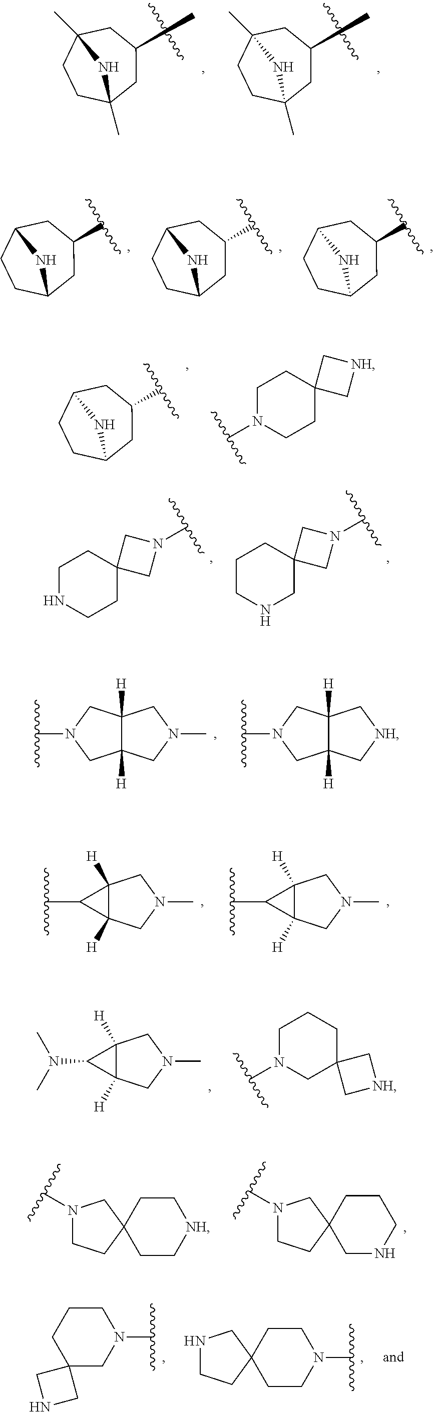

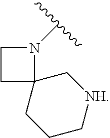 [SMILES: *N1CCC12CCCNC2]

In some embodiments, A is selected from

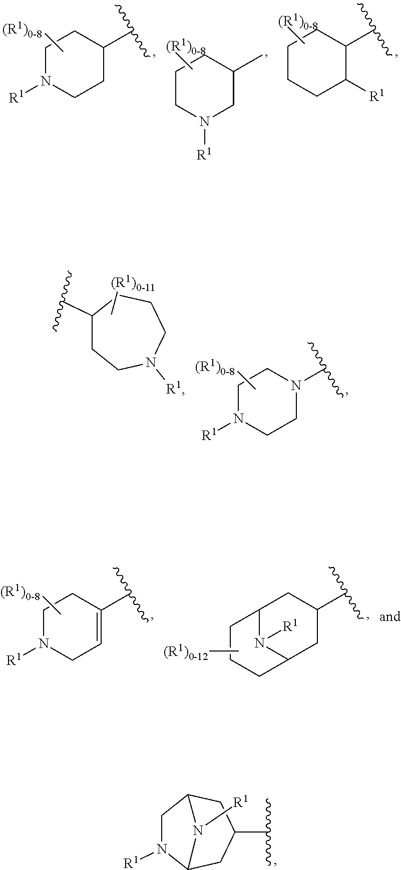

wherein R¹ is as defined herein.

In some embodiments, A is selected from,

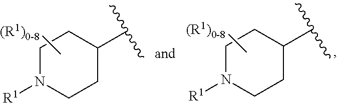

wherein R¹ is as defined herein.

In some embodiments, A is selected from

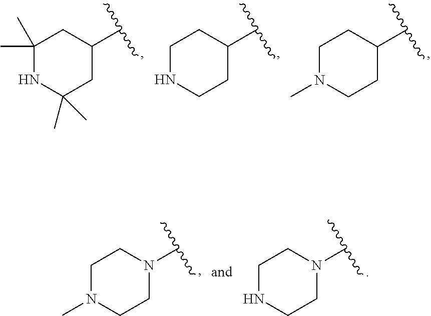

In some embodiments, A is heteroaryl. In some embodiments, A is a nitrogen-containing heteroaryl. In some embodiments, A is a bicyclic nitrogen-containing heteroaryl. In some embodiments, A is selected from

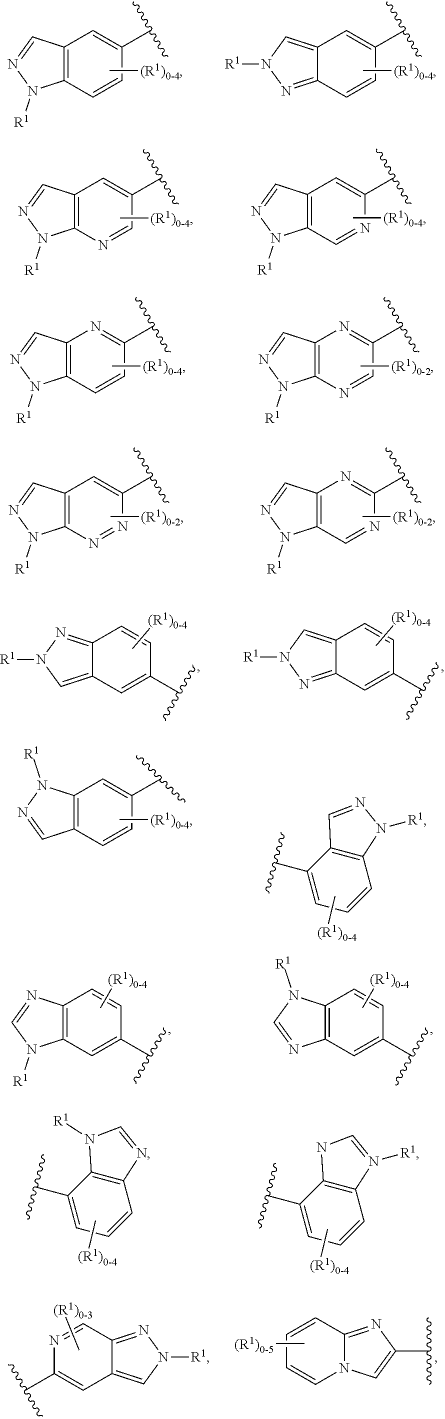

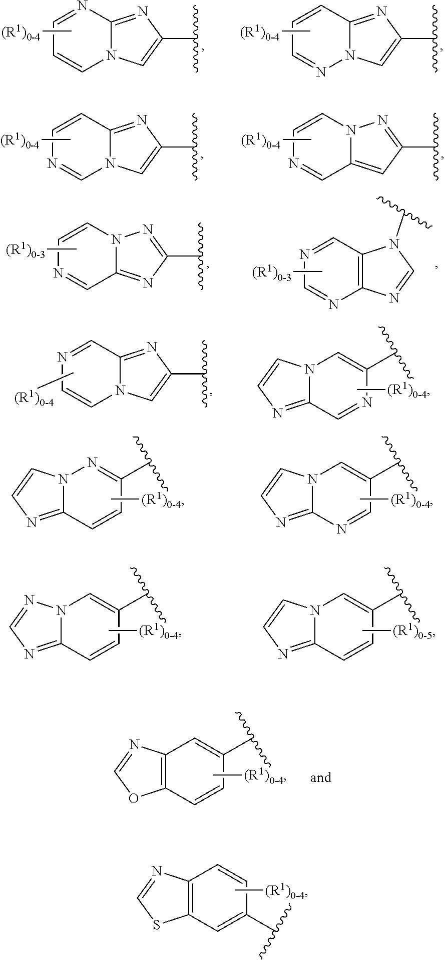

wherein R¹ is as defined herein.

In some embodiments, A is selected from

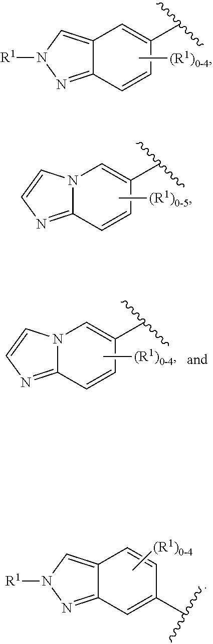

In some embodiments, A is

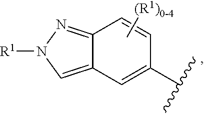

wherein R¹ is as defined herein. In some embodiments, A is selected from

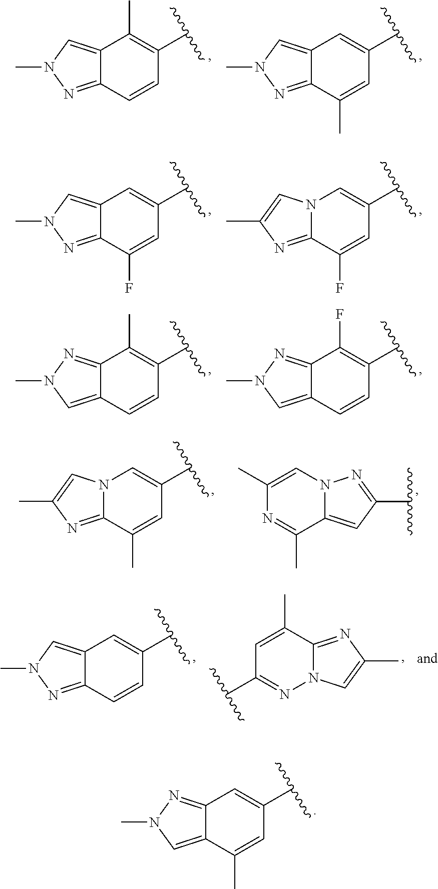

In some embodiments, A is

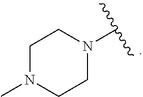 [SMILES: *N1CCN(C)CC1]

In some embodiments, A is

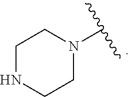 [SMILES: *N1CCNCC1]

In some embodiments, A is

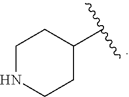 [SMILES: *C1CCNCC1]

In some embodiments, A is

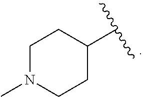 [SMILES: *C1CCN(C)CC1]

In some embodiments, A is

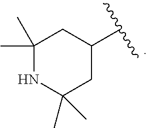 [SMILES: *C1CC(C)(C)NC(C)(C)C1]

In some embodiments, A is

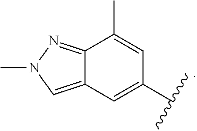 [SMILES: *c1cc(C)c2nn(C)cc2c1]

In some embodiments, A is

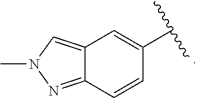 [SMILES: *c1ccc2nn(C)cc2c1]

In some embodiments, A is

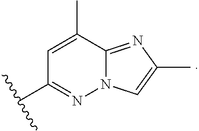 [SMILES: *c1cc(C)c2nc(C)cn2n1]

In some embodiments, A is

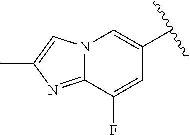 [SMILES: *c1cc(F)c2nc(C)cn2c1]

In some embodiments, B is heteroaryl. In some embodiments, B is a nitrogen-containing heteroaryl. In some embodiments, B is a bicyclic nitrogen-containing heteroaryl. In some embodiments, B is selected from

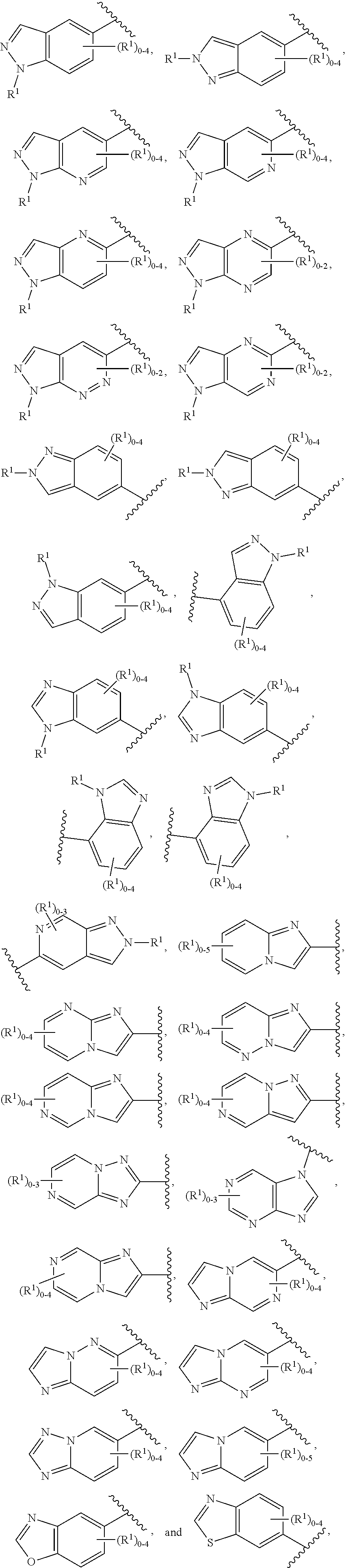

wherein R¹ is as defined herein. In some embodiments, B is selected from

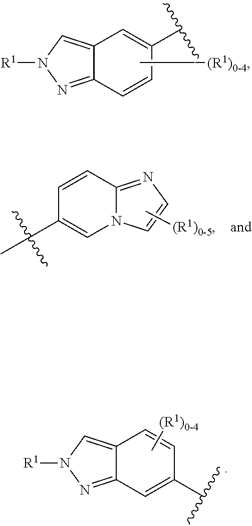

In some embodiments, B is

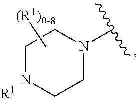

wherein R¹ is as defined herein.

In some embodiments, B is selected from

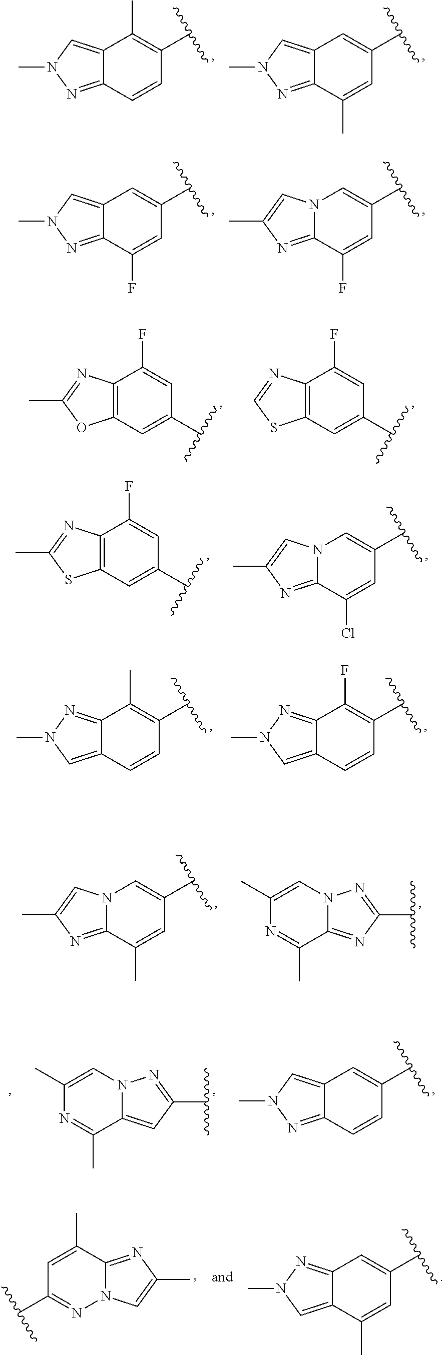

In some embodiments, B is heterocyclyl. In some embodiments, B is a nitrogen-containing heterocyclyl. In some embodiments, B is a monocyclic nitrogen-containing heterocyclyl or a bicyclic nitrogen-containing heterocyclyl.

In some embodiments, B is selected from

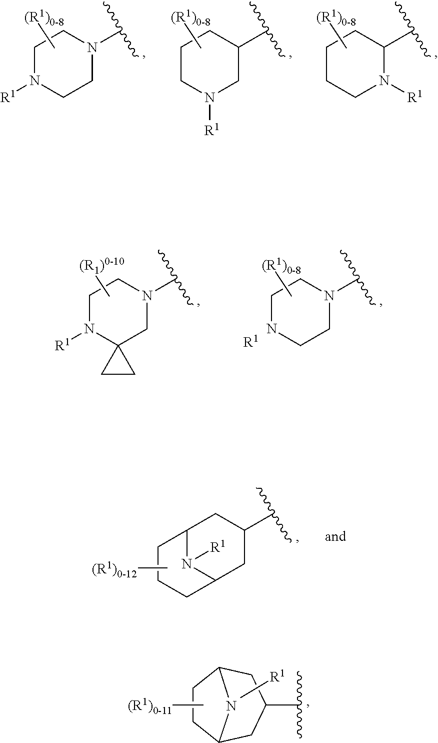

wherein R¹ is as defined herein. In some embodiments, B is selected from

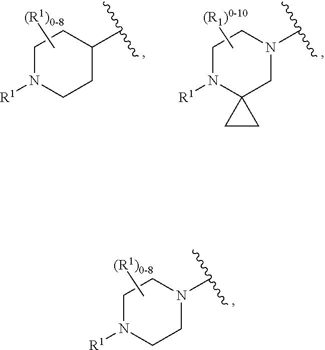

wherein R¹ is as defined herein. In some embodiments, B is selected from,

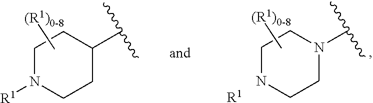

wherein R¹ is as defined herein.

In some embodiments, B is selected from

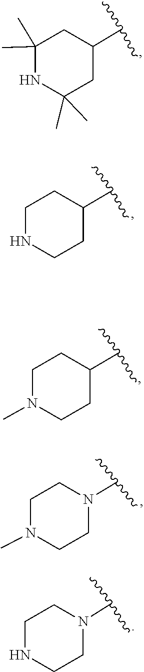

In some embodiments, B is

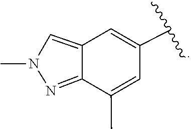

In some embodiments, B is

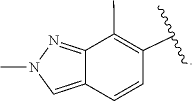 [SMILES: *c1ccc2cn(C)nc2c1C]

In some embodiments, B is

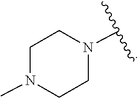 [SMILES: *N1CCN(C)CC1]

In some embodiments, B is

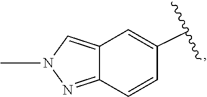 [SMILES: *c1ccc2nn(C)cc2c1]

In some embodiments, B is

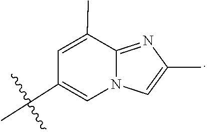 [SMILES: *Cc1cc(C)c2nc(C)cn2c1]

In some embodiments, B is

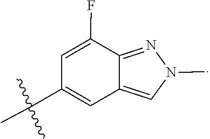 [SMILES: *Cc1cc(F)c2nn(C)cc2c1]

In some embodiments, B is

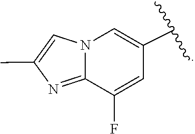 [SMILES: *c1cc(F)c2nc(C)cn2c1]

In some embodiments, B is

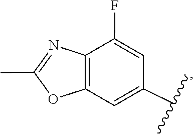 [SMILES: *c1cc(F)c2nc(C)oc2c1]

In some embodiments, B is

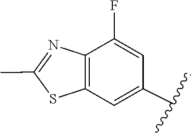 [SMILES: *c1cc(F)c2nc(C)sc2c1]

In some embodiments, B is

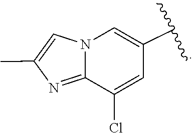 [SMILES: *c1cc(Cl)c2nc(C)cn2c1]

In some embodiments, B is

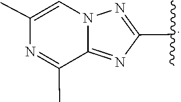 [SMILES: *c1nc2c(C)nc(C)cn2n1]

In some embodiments, B is

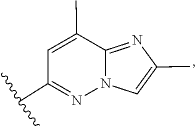 [SMILES: *c1cc(C)c2nc(C)cn2n1]

In some embodiments, B is

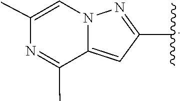 [SMILES: *c1cc2c(C)nc(C)cn2n1]

In some embodiments, B is

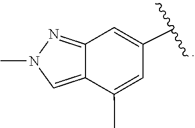 [SMILES: *c1cc(C)c2cn(C)nc2c1]

In some embodiments, B is

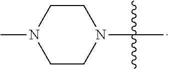

In some embodiments, B is

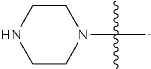

As generally described for Formulas (I), (III), and (V), each of L¹ and L² may independently be absent or refer to a C₁-C₆-alkylene, C₁-C₆-heteroalkylene, —O—, —C(O)—, —N(R⁸)—, —N(R⁸)C(O)—, or —C(O)N(R⁸)— group, wherein each alkylene and heteroalkylene is optionally substituted with one or more R⁹. In some embodiments, L¹ is absent or C₁-C₆-heteroalkylene. In some embodiments, L¹ is absent. In some embodiments, L¹ is C₁-C₆-heteroalkylene (e.g., —N(CH₃)—). In some embodiments, L² is absent or C₁-C₆-heteroalkylene. In some embodiments, L² is absent. In some embodiments, L² is C₁-C₆-heteroalkylene (e.g., —N(CH₃)—).

As generally described for Formula (I), each of W, X, and Z may independently be N or C(R³). In some embodiments, W is C(R³) (e.g., CH). In some embodiments, W is N. In some embodiments, X is C(R³) (e.g., CH). In some embodiments, X is N. In some embodiments, Z is C(R³) (e.g., CH). In some embodiments, Z is N. In some embodiments, each of W and X is independently C(R³) (e.g., CH). In some embodiments, each of W and Z is independently C(R³) (e.g., CH). In some embodiments, each of X and Z is independently C(R³) (e.g., CH). In some embodiments, each of W, X, and Z is independently C(R³) (e.g., CH).

As generally described for Formula (I), Y may be N, N(R^{4a}), C(R^{4b}), or C(R^{4b})(R^{4c}), wherein the dashed lines in the ring comprising Y may be single or double bonds as valency permits. In some embodiments, Y is N(R^{4a}) or C(R^{4b}). In some embodiments, Y is N(R^{4a}) (e.g., NH). In some embodiments, Y is C(R^{4b}) (e.g., CH).

In some embodiments, W is C(R³) and Y is N(R^{4a}). In some embodiments, W is CH and Y is NH. In some embodiments, X is C(R³) and Y is N(R^{4a}). In some embodiments, X is CH and Y is NH. In some embodiments, Z is C(R³) and Y is N(R^{4a}). In some embodiments, Z is CH and Y is NH. In some embodiments, W and X are independently C(R³) and Y is N(R^{4a}). In some embodiments, W and X are independently C(R³) and Y is NH. In some embodiments, W and Z are independently C(R³) and Y is N(R^{4a}). In some embodiments, W and Z are independently C(R³) and Y is NH. In some embodiments, X and Z are independently C(R³) and Y is N(R^{4a}). In some embodiments, X and Z are independently C(R³) and Y is NH. In some embodiments, each of W, X, and Z is independently C(R³) and Y is N(R^{4a}). In some embodiments, each of W, X, and Z is independently CH and Y is NH.

In some embodiments, W is C(R³) and Y is N. In some embodiments, W is CH and Y is N. In some embodiments, X is C(R³) and Y is N. In some embodiments, X is CH and Y is N. In some embodiments, Z is C(R³) and Y is N. In some embodiments, Z is CH and Y is N. In some embodiments, W and X are independently C(R³) and Y is N. In some embodiments, W and X are independently C(R³) and Y is N. In some embodiments, W and Z are independently C(R³) and Y is N. In some embodiments, W and Z are independently C(R³) and Y is N. In some embodiments, X and Z are independently C(R³) and Y is N. In some embodiments, X and Z are independently C(R³) and Y is N. In some embodiments, each of W, X, and Z is independently C(R³) and Y is N. In some embodiments, each of W, X, and Z is independently CH and Y is N.

In some embodiments, R² is absent.

In some embodiments, R¹ is C₁-C₆-alkyl. In some embodiments, R¹ is CH₃. In some embodiments, A is substituted with 0 or 1 In some embodiments, B is substituted with 0, 1, or 2 R¹.

In some embodiments of Formula (I), A is a bicyclic heteroaryl and B is a monocyclic heterocyclyl. In some embodiments of Formula (I), Z is N. In some embodiments of Formula (I), each of W, X, and Z is not independently C(R³), e.g., (CH). In some embodiments of Formula (I), the compound is not a compound disclosed in WO 2020/004594.

In some embodiments, the compound of Formula (I) is a compound of Formula (I-a):

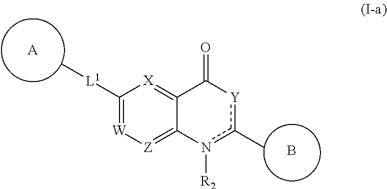

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L¹ is absent, C₁-C₆-alkylene, C₁-C₆-heteroalkylene, —O—, —C(O)—, —N(R⁸)—, —N(R⁸)C(O)—, or —C(O)N(R⁸)—, wherein each alkylene and heteroalkylene is optionally substituted with one or more R⁹; each of W, X, and Z is independently C(R³) or N; Y is N, N(R^{4a}), C(R^{4b}), or C(R^{4b})(R^{4c}), wherein the dashed lines in the ring comprising Y may be single or double bonds as valency permits; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁵; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵; R² is absent, hydrogen, or C₁-C₆-alkyl; R³ is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —C(O)R^{D}, or —C(O)OR^{D}; R^{4a} is hydrogen, C₁-C₆-alkyl, C₁-C₆-heteroalkyl, or C₁-C₆-haloalkyl; each of R^{4b} and R^{4c} is independently hydrogen, C₁-C₆-alkyl, C₁-C₆-heteroalkyl, C₁-C₆-haloalkyl, halo, or —OR^{A}; each R⁵ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁶; each R⁶ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R⁸ is independently hydrogen, C₁-C₆-alkyl, or C₁-C₆-haloalkyl; each R⁹ is independently C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each R^{B} and R^{C} is independently hydrogen, C₁-C₆ alkyl, C₁-C₆ heteroalkyl, cycloalkyl, heterocyclyl, —OR^{A}; or R^{B} and R^{C} together with the atom to which they are attached form a 3-7-membered heterocyclyl ring optionally substituted with one or more R¹⁰; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R¹⁰ is independently C₁-C₆-alkyl or halo; and x is 0, 1, or 2.

In some embodiments, A is heterocyclyl optionally substituted with one or more R¹. In some embodiments, A is monocyclic nitrogen-containing heterocyclyl. In some embodiments, A is optionally substituted piperidinyl. In some embodiments, A is selected from

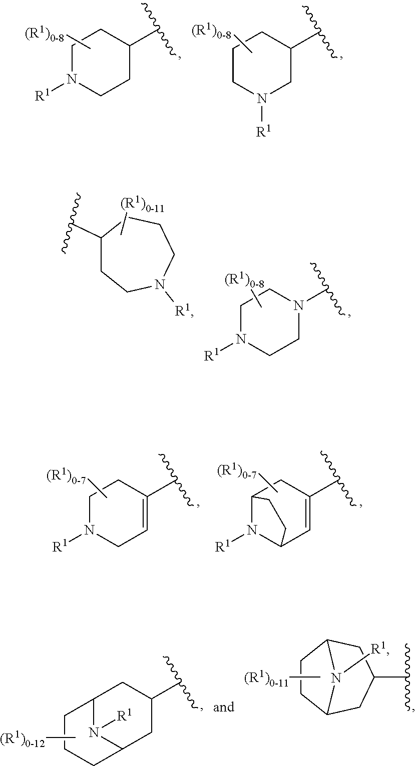

wherein R¹ is as defined herein.

In some embodiments, A is selected from,

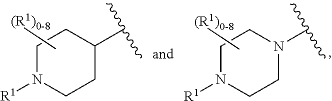

wherein R¹ is as defined herein. In some embodiments, A is selected from

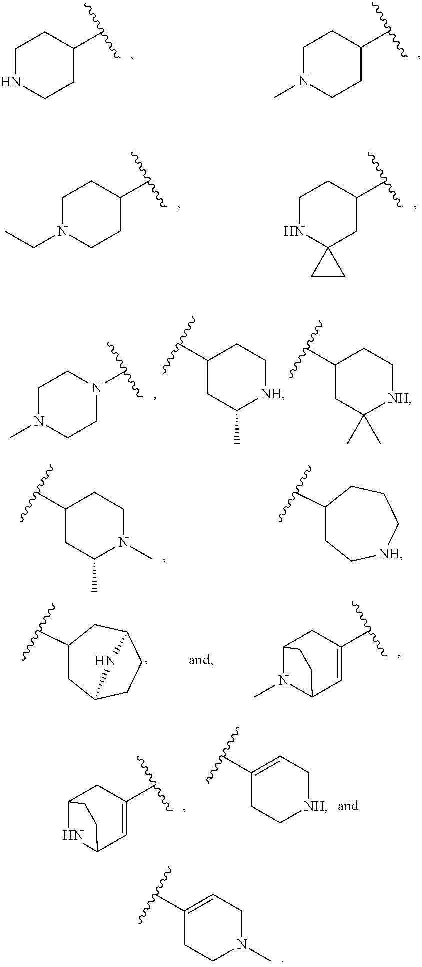

In some embodiments, A is selected from

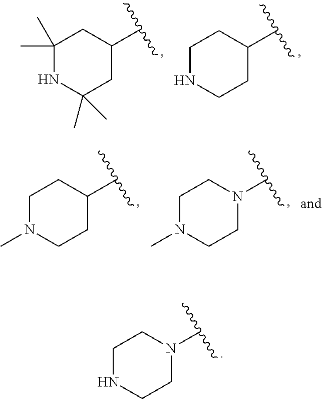

In some embodiments, A is heteroaryl. In some embodiments, A is a nitrogen-containing heteroaryl. In some embodiments, A is a bicyclic nitrogen-containing heteroaryl.

In some embodiments, A is selected from

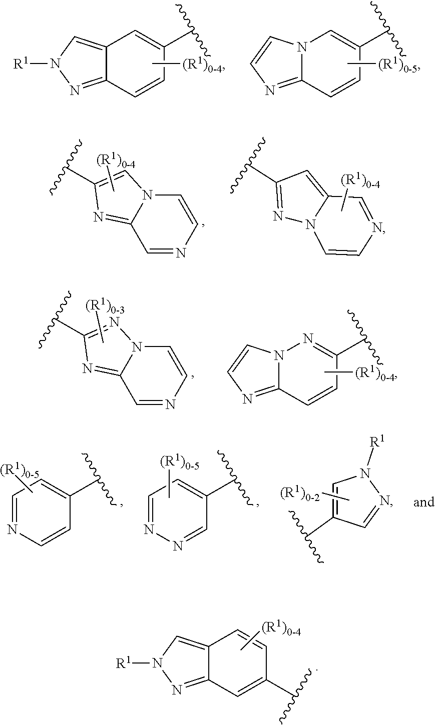

In some embodiments, A is

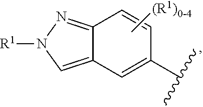

wherein R¹ is as defined herein. In some embodiments, A is selected from

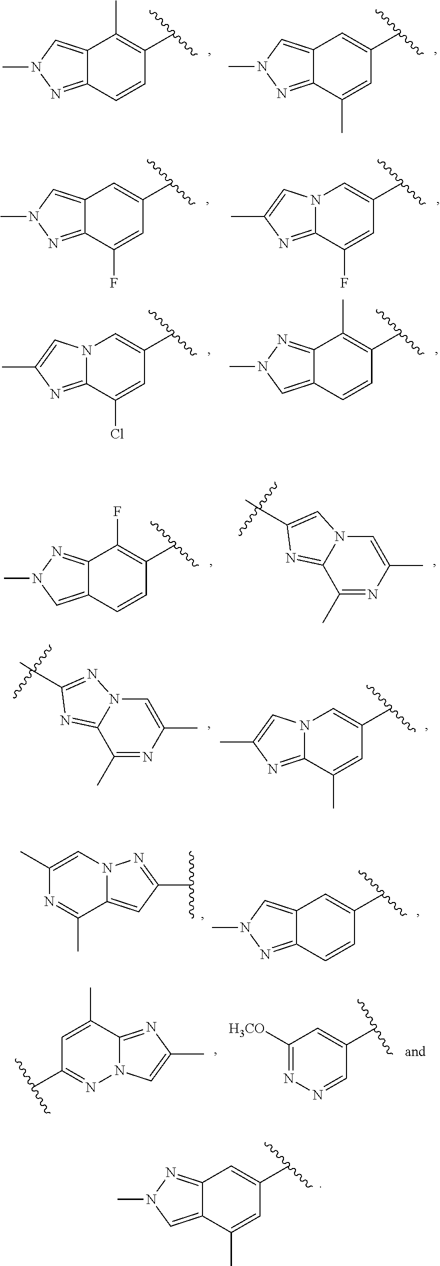

In some embodiments, A is

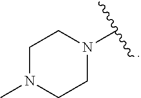 [SMILES: *N1CCN(C)CC1]

In some embodiments, A is

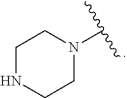 [SMILES: *N1CCNCC1]

In some embodiments, A is

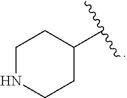 [SMILES: *C1CCNCC1]

In some embodiments, A is

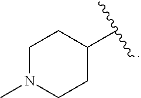 [SMILES: *C1CCN(C)CC1]

In some embodiments, A is

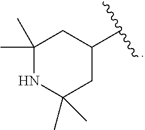 [SMILES: *C1CC(C)(C)NC(C)(C)C1]

In some embodiments, A is

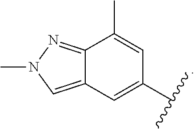 [SMILES: *c1cc(C)c2nn(C)cc2c1]

In some embodiments, A is

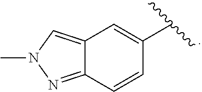 [SMILES: *c1ccc2nn(C)cc2c1]

In some embodiments, A is

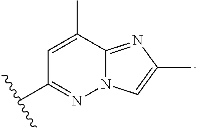 [SMILES: *c1cc(C)c2nc(C)cn2n1]

In some embodiments, A is

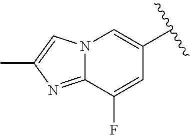 [SMILES: *c1cc(F)c2nc(C)cn2c1]

In some embodiments, B is heteroaryl. In some embodiments, B is a nitrogen-containing heteroaryl. In some embodiments, B is a bicyclic nitrogen-containing heteroaryl. In some embodiments, B is selected from

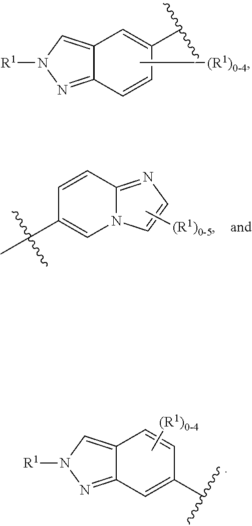

In some embodiments, B is

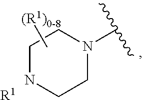

wherein R¹ is as defined herein.

In some embodiments, B is selected from

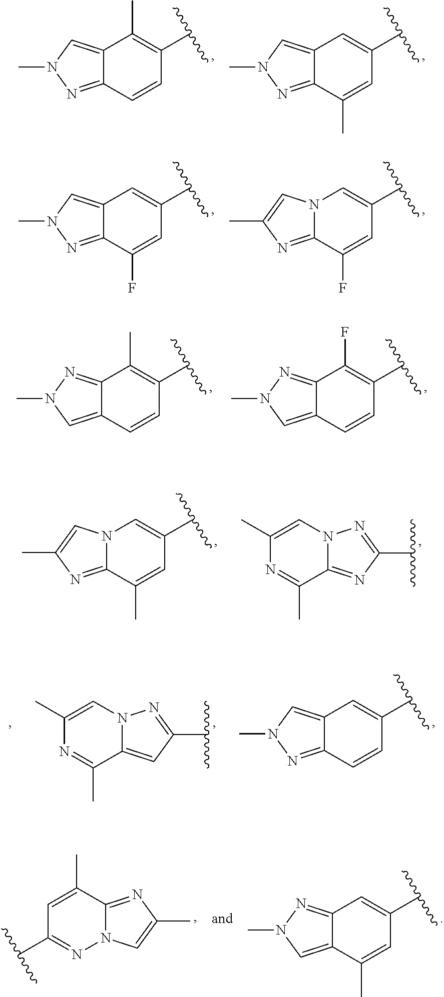

In some embodiments, B is heterocyclyl. In some embodiments, B is a nitrogen-containing heterocyclyl. In some embodiments, B is a monocyclic nitrogen-containing heterocyclyl or a bicyclic nitrogen-containing heterocyclyl. In some embodiments, B is selected from

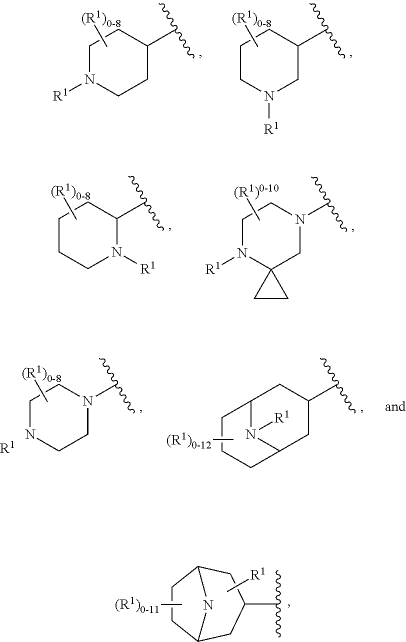

wherein R¹ is as defined herein. In some embodiments, B is selected from

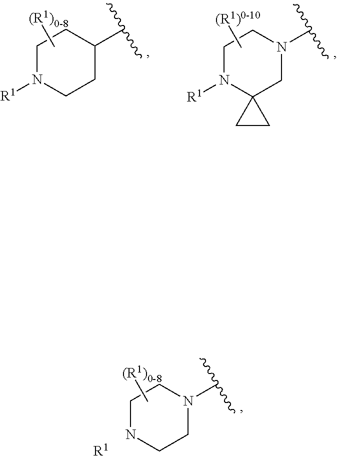

wherein R¹ is as defined herein. In some embodiments, B is selected from,

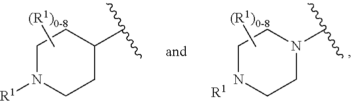

wherein R¹ is as defined herein.

In some embodiments, B is selected from

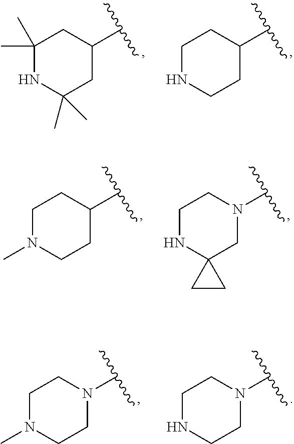

In some embodiments, B is

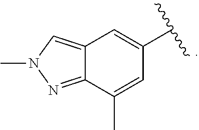 [SMILES: *c1cc(C)c2nn(C)cc2c1]

In some embodiments, B is

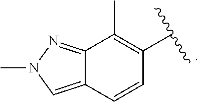 [SMILES: *c1ccc2cn(C)nc2c1C]

In some embodiments, B is

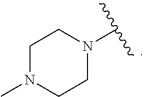 [SMILES: *N1CCN(C)CC1]

In some embodiments, B is

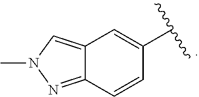 [SMILES: *c1ccc2nn(C)cc2c1]

In some embodiments, B is

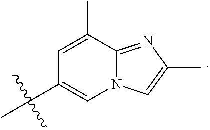 [SMILES: *c1cc(C)c2nc(C)cn2c1]

In some embodiments, B is

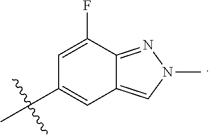 [SMILES: *c1cc(F)c2nn(C)cc2c1]

In some embodiments, B is

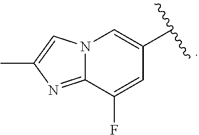 [SMILES: *c1cc(F)c2nc(C)cn2c1]

In some embodiments, B is

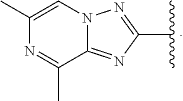 [SMILES: *c1nc2c(C)nc(C)cn2n1]

In some embodiments, B is

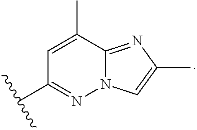 [SMILES: *c1cc(C)c2nc(C)cn2n1]

In some embodiments, B is

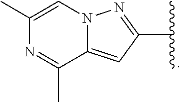 [SMILES: *c1cc2c(C)nc(C)cn2n1]

In some embodiments, B is

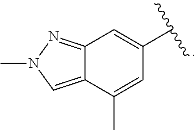 [SMILES: *c1cc(C)c2cn(C)nc2c1]

In some embodiments, B is

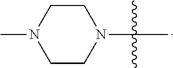 [SMILES: *N1CCN(C)CC1]

In some embodiments, B is

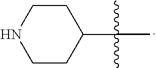 [SMILES: *C1CCNCC1]

In some embodiments, B is

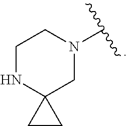 [SMILES: *N1CCNC2(CC2)C1]

In some embodiments, L¹ is absent or N(CH₃). In some embodiments, L¹ is absent. In some embodiments, L¹ is N(CH₃).

In some embodiments, each of W, X, and Z may independently be N or C(R³). In some embodiments, W is C(R³) (e.g., CH). In some embodiments, W is N. In some embodiments, X is C(R³) (e.g., CH). In some embodiments, X is N. In some embodiments, Z is C(R³) (e.g., CH). In some embodiments, Z is N. In some embodiments, each of W and X is independently C(R³) (e.g., CH). In some embodiments, each of W and Z is independently C(R³) (e.g., CH). In some embodiments, each of X and Z is independently C(R³) (e.g., CH). In some embodiments, each of W, X, and Z is independently C(R³) (e.g., CH).

In some embodiments, R^{4a} is hydrogen or C₁-C₆ alkyl. In some embodiments, R^{4a} is hydrogen.

In some embodiments, R¹ is C₁-C₆-alkyl. In some embodiments, R¹ is CH₃. In some embodiments, A is substituted with 0 or 1 R¹. In some embodiments, B is substituted with 0, 1, or 2 R¹.

In some embodiments, A is a bicyclic heteroaryl and B is a monocyclic heterocyclyl. In some embodiments of Formula (I), Z is N. In some embodiments of Formula (I), each of W, X, and Z is not independently C(R³), e.g., (CH).

In some embodiments, the compound of Formula (I) is a compound of Formula (I-b):

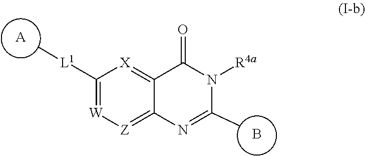

or a pharmaceutically acceptable salt, solvate, hydrate, tautomer, or stereoisomer thereof, A and B are each independently cycloalkyl, heterocyclyl, aryl, or heteroaryl, each of which is optionally substituted with one or more R¹; L¹ is absent, C₁-C₆-alkylene, C₁-C₆-heteroalkylene, —O—, —C(O)—, —N(R⁸)—, —N(R⁸)C(O)—, or —C(O)N(R⁸)—, wherein each alkylene and heteroalkylene is optionally substituted with one or more R⁹; each of W, X, and Z is independently C(R³) or N; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁵; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵; R³ is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —C(O)R^{D}, or —C(O)OR^{D} ; R^{4a} is hydrogen, C₁-C₆-alkyl, C₁-C₆-heteroalkyl, or C₁-C₆-haloalkyl; each R⁵ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁶; each R⁶ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R⁸ is independently hydrogen, C₁-C₆-alkyl, or C₁-C₆-haloalkyl; each R⁹ is independently C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each R^{B} and R^{C} is independently hydrogen, C₁-C₆ alkyl, C₁-C₆ heteroalkyl, cycloalkyl, heterocyclyl, —OR^{A}; or R^{B} and R^{C} together with the atom to which they are attached form a 3-7-membered heterocyclyl ring optionally substituted with one or more R¹⁰; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R¹⁰ is independently C₁-C₆-alkyl or halo; and x is 0, 1, or 2.

In some embodiments, A is heterocyclyl optionally substituted with one or more R¹. In some embodiments, A is monocyclic nitrogen-containing heterocyclyl. In some embodiments, A is optionally substituted piperidinyl. In some embodiments, A is selected from

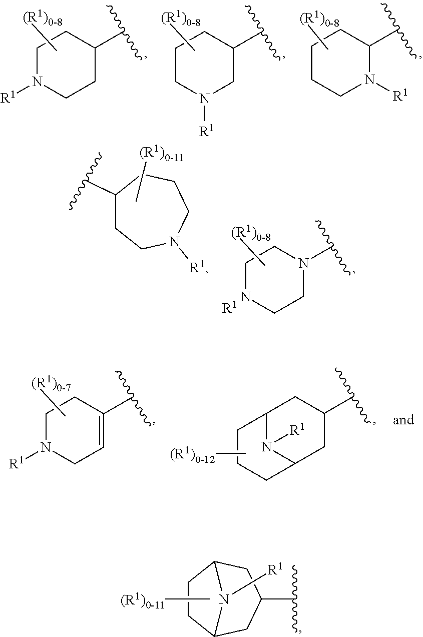

wherein R¹ is as defined herein.

In some embodiments, A is selected from,

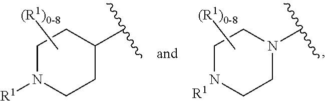

wherein R¹ is as defined herein.

In some embodiments, A is selected from

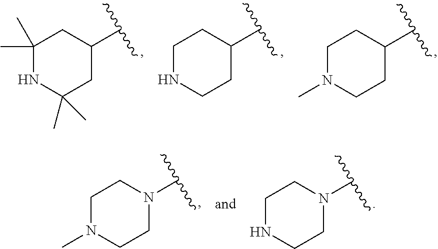

In some embodiments, A is heteroaryl. In some embodiments, A is a nitrogen-containing heteroaryl. In some embodiments, A is a bicyclic nitrogen-containing heteroaryl.

In some embodiments, A is selected from

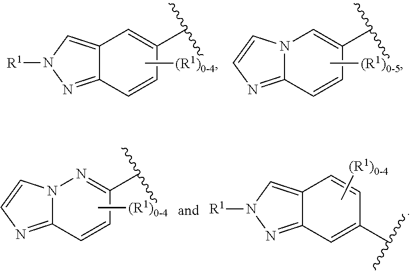

In some embodiments, A is

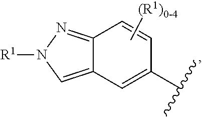

wherein R¹ is as defined herein. In some embodiments, A is selected from

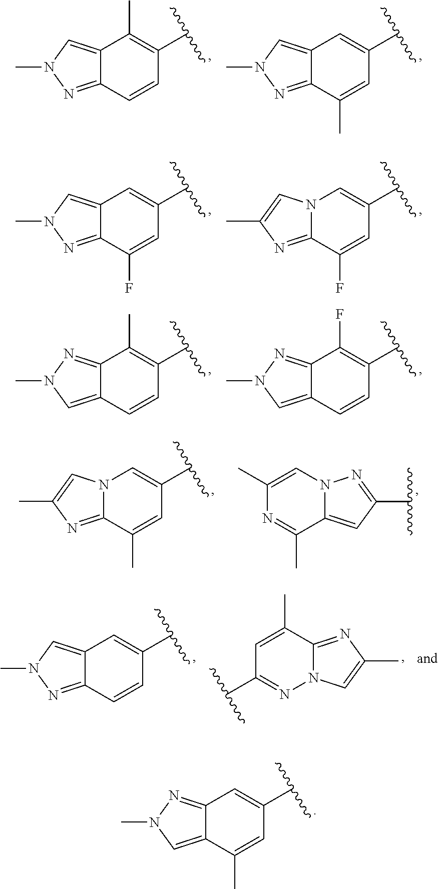

In some embodiments, A is

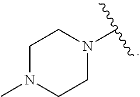 [SMILES: *N1CCN(C)CC1]

In some embodiments, A is

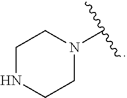 [SMILES: *N1CCNCC1]

In some embodiments, A is

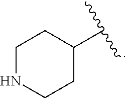 [SMILES: *C1CCNCC1]

In some embodiments, A is

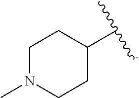 [SMILES: *C1CCN(C)CC1]

In some embodiments, A is

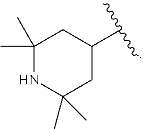 [SMILES: *C1CC(C)(C)NC(C)(C)C1]

In some embodiments, A is

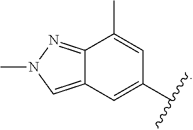 [SMILES: *c1cc(C)c2nn(C)cc2c1]

In some embodiments, A is

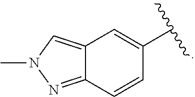 [SMILES: *c1ccc2nn(C)cc2c1]

In some embodiments, A is

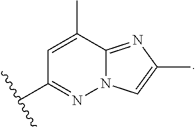 [SMILES: *c1cc(C)c2nc(C)cn2n1]

In some embodiments, A is

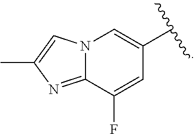 [SMILES: *c1cc(F)c2nc(C)cn2c1]

In some embodiments, B is heteroaryl. In some embodiments, B is a nitrogen-containing heteroaryl. In some embodiments, B is a bicyclic nitrogen-containing heteroaryl. In some embodiments, B is selected from

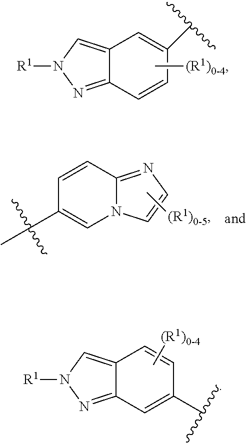

In some embodiments, B is

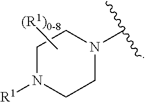

wherein R¹ is as defined herein.

In some embodiments, B is selected from

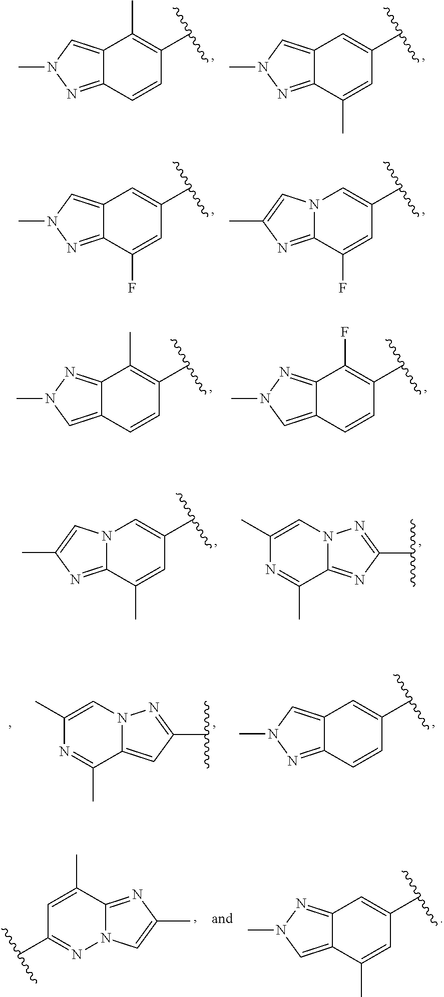

In some embodiments, B is heterocyclyl. In some embodiments, B is a nitrogen-containing heterocyclyl. In some embodiments, B is a monocyclic nitrogen-containing heterocyclyl or a bicyclic nitrogen-containing heterocyclyl. In some embodiments, B is selected from

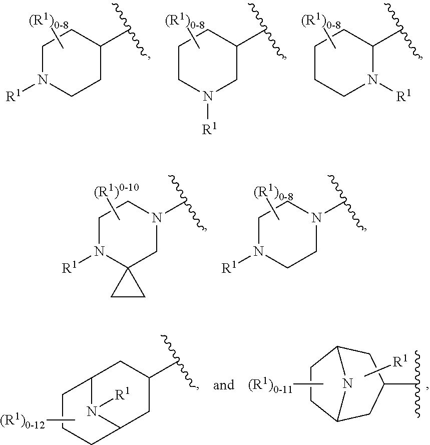

wherein R¹ is as defined herein. In some embodiments, B is selected from

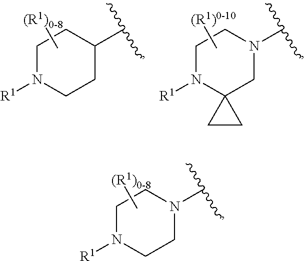

wherein R¹ is as defined herein. In some embodiments, B is selected from,

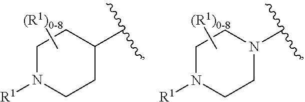

wherein R¹ is as defined herein.

In some embodiments, B is selected from

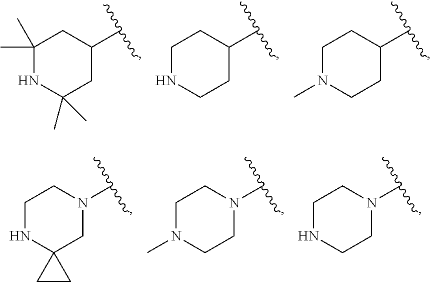

In some embodiments, B is

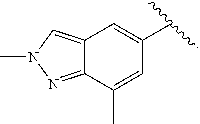 [SMILES: *c1cc(C)c2nn(C)cc2c1]

In some embodiments, B is

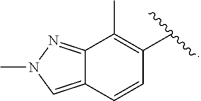 [SMILES: *c1ccc2cn(C)nc2c1C]

In some embodiments, B is

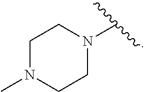 [SMILES: *N1CCN(C)CC1]

In some embodiments, B is

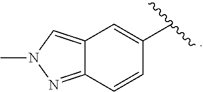 [SMILES: *c1ccc2nn(C)cc2c1]

In some embodiments, B is

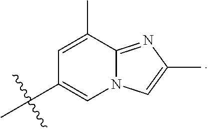 [SMILES: *c1cc(C)c2nc(C)cn2c1]

In some embodiments, B is

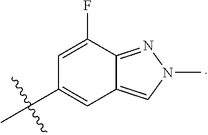 [SMILES: *c1cc(F)c2nn(C)cc2c1]

In some embodiments, B is

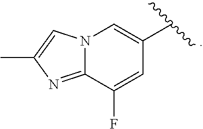 [SMILES: *c1cc(F)c2nc(C)cn2c1]

In some embodiments, B is

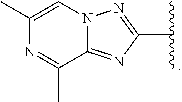 [SMILES: *c1nc2c(C)nc(C)cn2n1]

In some embodiments, B is

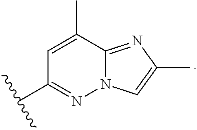 [SMILES: *c1cc(C)c2nc(C)cn2n1]

In some embodiments, B is

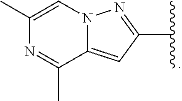 [SMILES: *c1cc2c(C)nc(C)cn2n1]

In some embodiments, B is

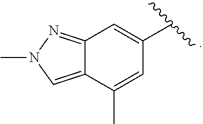 [SMILES: *c1cc(C)c2cn(C)nc2c1]

In some embodiments, B is

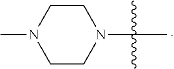 [SMILES: *N1CCN(C)CC1]

In some embodiments, B is

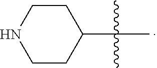 [SMILES: *C1CCNCC1]

In some embodiments, B is

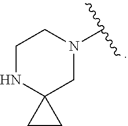 [SMILES: *N1CCNC2(CC2)C1]

In some embodiments, L¹ is absent or N(CH₃). In some embodiments, L¹ is absent. In some embodiments, L¹ is N(CH₃).

In some embodiments, each of W, X, and Z may independently be N or C(R³). In some embodiments, W is C(R³) (e.g., CH). In some embodiments, W is N. In some embodiments, X is C(R³) (e.g., CH). In some embodiments, X is N. In some embodiments, Z is C(R³) (e.g., CH). In some embodiments, Z is N. In some embodiments, each of W and X is independently C(R³) (e.g., CH). In some embodiments, each of W and Z is independently C(R³) (e.g., CH). In some embodiments, each of X and Z is independently C(R³) (e.g., CH). In some embodiments, each of W, X, and Z is independently C(R³) (e.g., CH).

In some embodiments, R^{4a} is hydrogen or C₁-C₆ alkyl. In some embodiments, R^{4a} is hydrogen.

In some embodiments, R¹ is C₁-C₆-alkyl. In some embodiments, R¹ is CH₃. In some embodiments, A is substituted with 0 or 1 In some embodiments, B is substituted with 0, 1, or 2 R¹.

In some embodiments, A is a bicyclic heteroaryl and B is a monocyclic heterocyclyl. In some embodiments of Formula (I), Z is N. In some embodiments of Formula (I), each of W, X, and Z is not independently C(R³), e.g., (CH).

In some embodiments, the compound of Formula (I) is a compound of Formula (I-c):

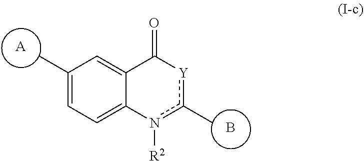

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; Y is N, N(R^{4a}), C(R^{4b}), or C(R^{4b})(R^{4c}), wherein the dashed lines in the ring comprising Y may be single or double bonds as valency permits; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁵; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵; R² is absent, hydrogen, or C₁-C₆-alkyl; R^{4a} is hydrogen, C₁-C₆-alkyl, C₁-C₆-heteroalkyl, or C₁-C₆-haloalkyl; each of R^{4b} and R^{4c} is independently hydrogen, C₁-C₆-alkyl, C₁-C₆-heteroalkyl, C₁-C₆-haloalkyl, halo, or —OR^{A}; each R⁵ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C}, NR^{B}C(O)R^{D}, —NO₂, —C(O)NR^{B}R^{C}, —C(O)R^{D}, C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁶; each R⁶ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each R^{B} and R^{C} is independently hydrogen, C₁-C₆ alkyl, C₁-C₆ heteroalkyl, cycloalkyl, heterocyclyl, —OR^{A}; or R^{B} and R^{C} together with the atom to which they are attached form a 3-7-membered heterocyclyl ring optionally substituted with one or more R¹⁰; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each le° is independently C₁-C₆-alkyl or halo; and x is 0, 1, or 2.

In some embodiments, A is heterocyclyl optionally substituted with one or more R¹. In some embodiments, A is monocyclic nitrogen-containing heterocyclyl. In some embodiments, A is optionally substituted piperidinyl. In some embodiments, A is selected from

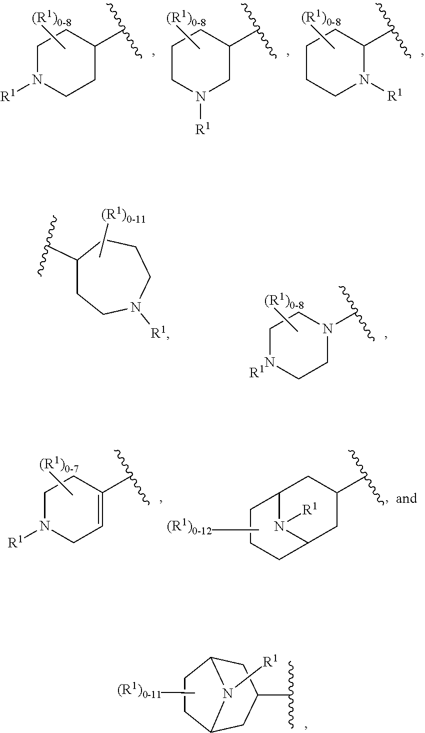

wherein R¹ is as defined herein.

In some embodiments, A is selected from,

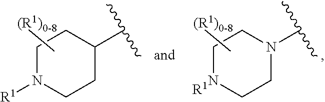

wherein R¹ is as defined herein.

In some embodiments, A is selected from

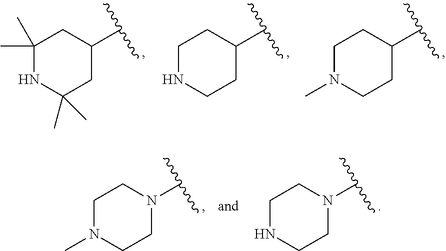

In some embodiments, A is heteroaryl. In some embodiments, A is a nitrogen-containing heteroaryl. In some embodiments, A is a bicyclic nitrogen-containing heteroaryl.

In some embodiments, A is selected from

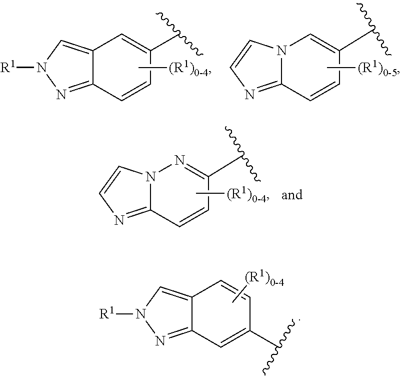

In some embodiments, A is

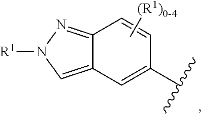

wherein R¹ is as defined herein. In some embodiments, A is selected from

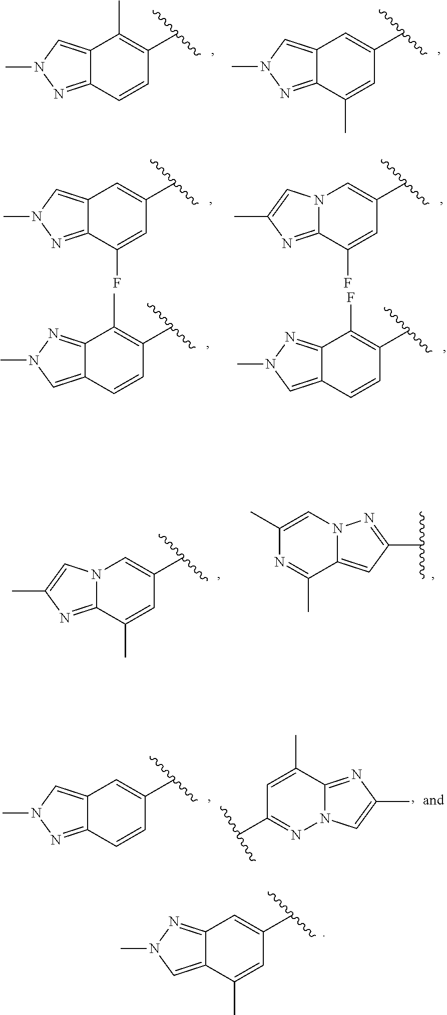

In some embodiments, A is

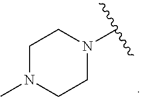 [SMILES: *N1CCN(C)CC1]

In some embodiments, A is

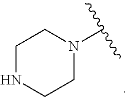 [SMILES: *N1CCNCC1]

In some embodiments, A is

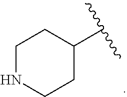 [SMILES: *C1CCNCC1]

In some embodiments, A is

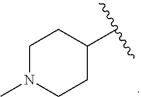 [SMILES: *C1CCN(C)CC1]

In some embodiments, A is

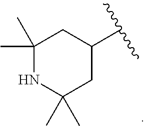 [SMILES: *C1CC(C)(C)NC(C)(C)C1]

In some embodiments, A is

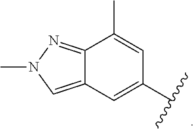 [SMILES: *c1cc(C)c2nn(C)cc2c1]

In some embodiments, A is

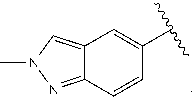 [SMILES: *c1ccc2nn(C)cc2c1]

In some embodiments, A is

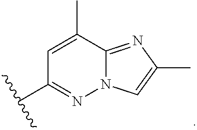 [SMILES: *c1cc(C)c2nc(C)cn2n1]

In some embodiments, A is

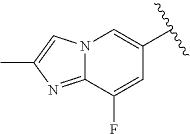 [SMILES: *c1cc(F)c2nc(C)cn2c1]

In some embodiments, B is heteroaryl. In some embodiments, B is a nitrogen-containing heteroaryl. In some embodiments, B is a bicyclic nitrogen-containing heteroaryl. In some embodiments, B is selected from

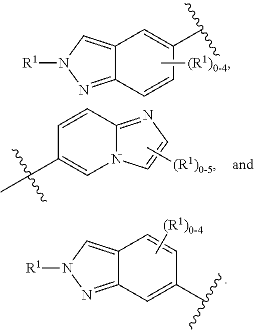

In some embodiments, B is

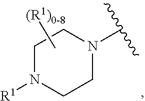

wherein R¹ is as defined herein.

In some embodiments, B is selected from

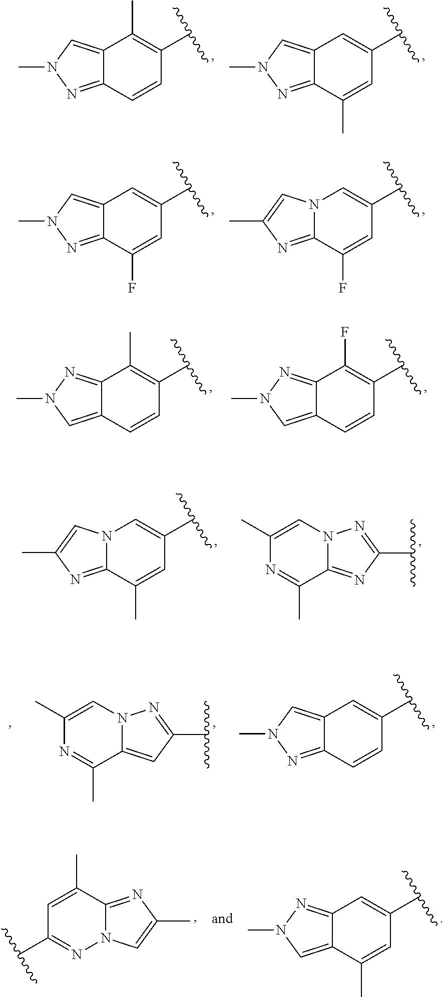

In some embodiments, B is heterocyclyl. In some embodiments, B is a nitrogen-containing heterocyclyl. In some embodiments, B is a monocyclic nitrogen-containing heterocyclyl or a bicyclic nitrogen-containing heterocyclyl. In some embodiments, B is selected from

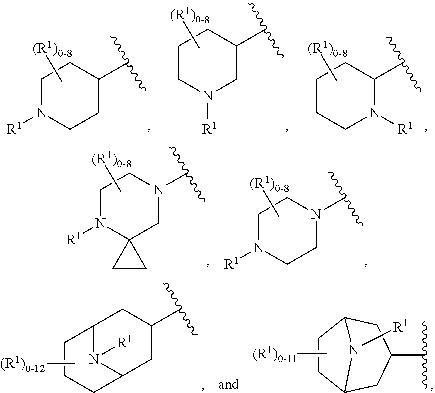

wherein R¹ is as defined herein. In some embodiments, B is selected from

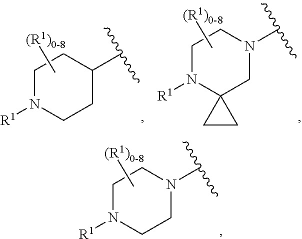

wherein R¹ is as defined herein. In some embodiments, B is selected from,

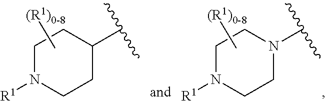

wherein R¹ is as defined herein.

In some embodiments, B is selected from

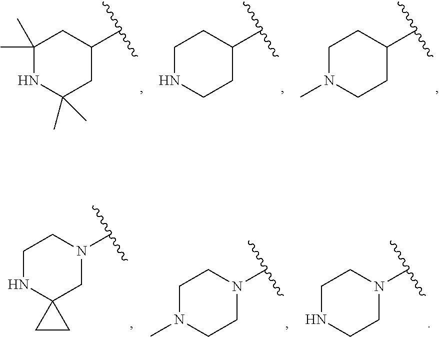

In some embodiments, B is

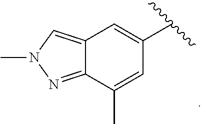 [SMILES: *c1cc(C)c2nn(C)cc2c1]

In some embodiments, B is

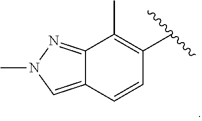 [SMILES: *c1ccc2cn(C)nc2c1C]

In some embodiments, B is

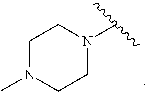 [SMILES: *N1CCN(C)CC1]

In some embodiments, B is

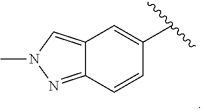 [SMILES: *c1ccc2nn(C)cc2c1]

In some embodiments, B is

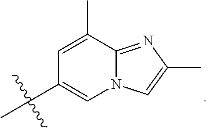 [SMILES: *c1cc(C)c2nc(C)cn2c1]

In some embodiments, B is

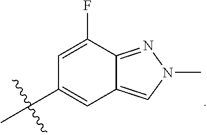 [SMILES: *c1cc(F)c2nn(C)cc2c1]

In some embodiments, B is

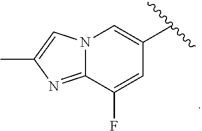 [SMILES: *c1cc(F)c2nc(C)cn2c1]

In some embodiments, B is

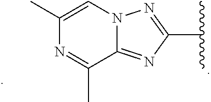 [SMILES: *c1nc2c(C)nc(C)cn2n1]

In some embodiments, B is

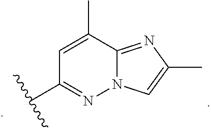 [SMILES: *c1cc(C)c2nc(C)cn2n1]

In some embodiments, B is

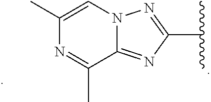 [SMILES: *c1nc2c(C)nc(C)cn2n1]

In some embodiments, B is

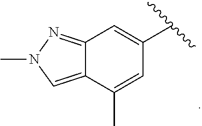 [SMILES: *c1cc(C)c2cn(C)nc2c1]

In some embodiments, B is

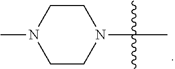 [SMILES: *N1CCN(C)CC1]

In some embodiments, B is

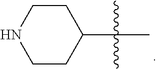 [SMILES: *C1CCNCC1]

In some embodiments, B is

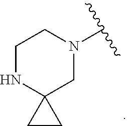 [SMILES: *N1CCNC2(CC2)C1]

As generally described, Y may be N, N(R^{4a}), C(R^{4b}), or C(R^{4b})(R^{4c}), wherein the dashed lines in the ring comprising Y may be single or double bonds as valency permits. In some embodiments, Y is N(R^{4a}) or C(R^{4b}). In some embodiments, Y is N(R^{4a}) (e.g., NH). In some embodiments, Y is C(R^{4b}) (e.g., CH).

In some embodiments, R² is absent.

In some embodiments, R¹ is C₁-C₆-alkyl. In some embodiments, R¹ is CH₃. In some embodiments, A is substituted with 0 or 1 In some embodiments, B is substituted with 0, 1, or 2 R¹.

In some embodiments, A is a bicyclic heteroaryl and B is a monocyclic heterocyclyl. In some embodiments of Formula (I), Z is N. In some embodiments of Formula (I), each of W, X, and Z is not independently C(R³), e.g., (CH).

In some embodiments, the compound of Formula (I) is a compound of Formula (I-d):

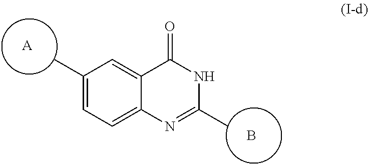

or a pharmaceutically acceptable salt, solvate, hydrate, tautomer, or stereoisomer thereof, wherein A is a monocyclic nitrogen-containing heterocyclyl optionally substituted with one or more R¹; B is a bicyclic nitrogen-containing heteroaryl optionally substituted with one or more R¹; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁵; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵; each R⁵ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁶; each R⁶ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each R^{B} and R^{C} is independently hydrogen, C₁-C₆ alkyl, C₁-C₆ heteroalkyl, cycloalkyl, heterocyclyl, —OR^{A}; or R^{B} and R^{C} together with the atom to which they are attached form a 3-7-membered heterocyclyl ring optionally substituted with one or more R¹⁰; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each le° is independently C₁-C₆-alkyl or halo; and x is 0, 1, or 2.

In some embodiments, A is heterocyclyl optionally substituted with one or more R¹. In some embodiments, A is monocyclic nitrogen-containing heterocyclyl. In some embodiments, A is optionally substituted piperidinyl. In some embodiments, A is selected from

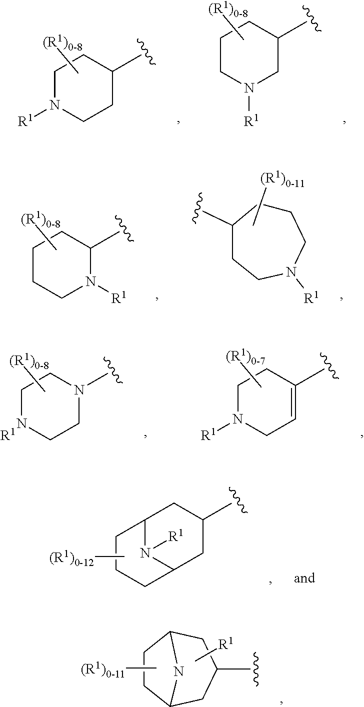

wherein R¹ is as defined herein.

In some embodiments, A is selected from,

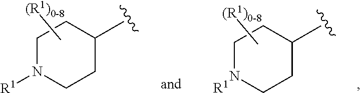

wherein R¹ is as defined herein.

In some embodiments, A is selected from

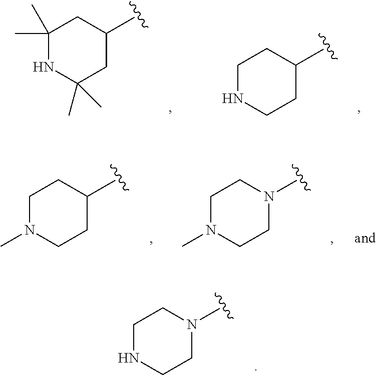

In some embodiments, A is heteroaryl. In some embodiments, A is a nitrogen-containing heteroaryl. In some embodiments, A is a bicyclic nitrogen-containing heteroaryl.

In some embodiments, A is selected from

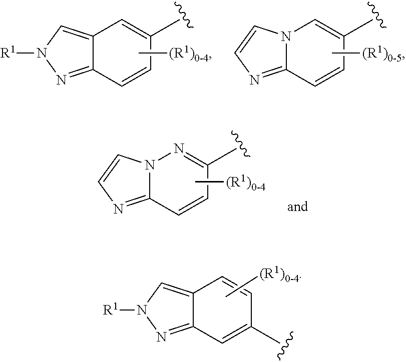

In some embodiments, A is

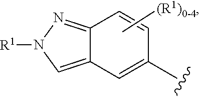

wherein R¹ is as defined herein. In some embodiments, A is selected from

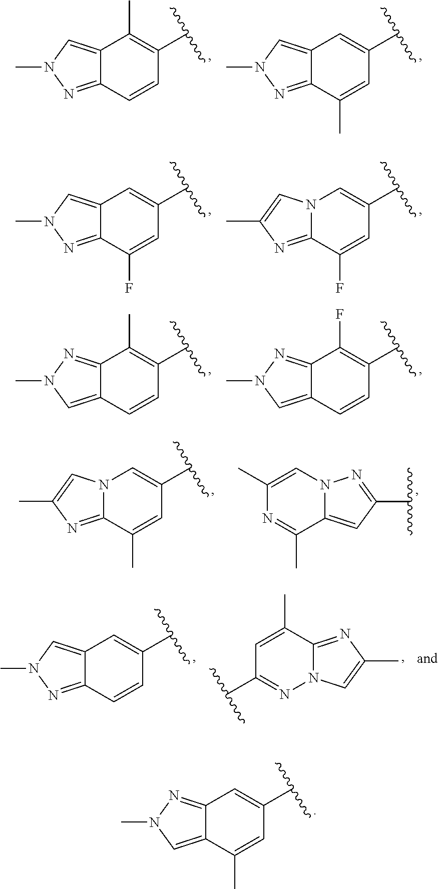

In some embodiments, A is

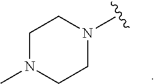

In some embodiments, A is

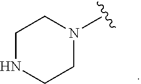

In some embodiments, A is

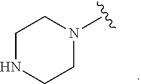

In some embodiments, A is

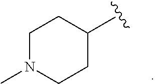

In some embodiments, A is

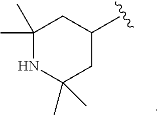

In some embodiments, A is

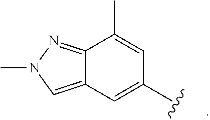

In some embodiments, A is

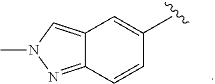

In some embodiments, A is

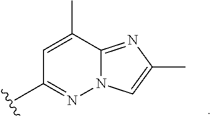

In some embodiments, A is

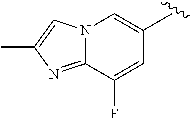

In some embodiments, B is heteroaryl. In some embodiments, B is a nitrogen-containing heteroaryl. In some embodiments, B is a bicyclic nitrogen-containing heteroaryl. In some embodiments, B is selected from

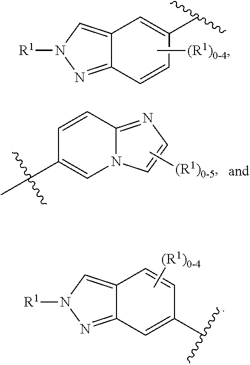

In some embodiments, B is

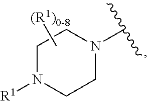

wherein R¹ is as defined herein.

In some embodiments, B is selected from

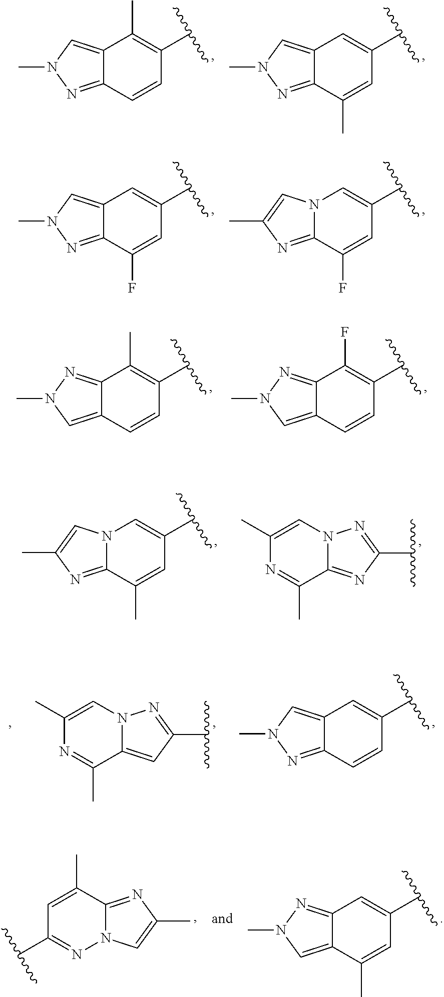

In some embodiments, B is heterocyclyl. In some embodiments, B is a nitrogen-containing heterocyclyl. In some embodiments, B is a monocyclic nitrogen-containing heterocyclyl or a bicyclic nitrogen-containing heterocyclyl. In some embodiments, B is selected from

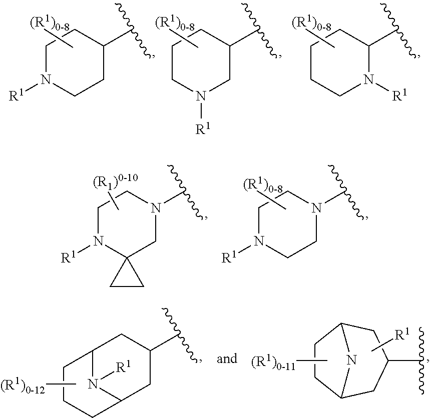

wherein R¹ is as defined herein. In some embodiments, B is selected from

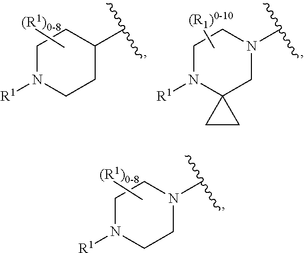

wherein R¹ is as defined herein. In some embodiments, B is selected from,

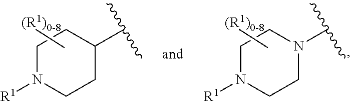

wherein R¹ is as defined herein.

In some embodiments, B is selected from

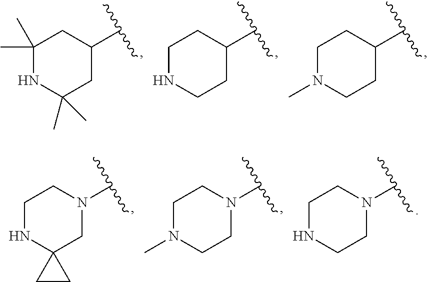

In some embodiments, B is

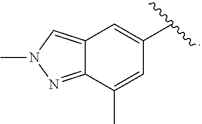 [SMILES: *c1cc(C)c2nn(C)cc2c1]

In some embodiments, B is

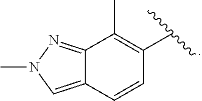 [SMILES: *c1ccc2cn(C)nc2c1C]

In some embodiments, B is

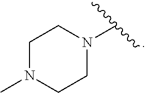 [SMILES: *N1CCN(C)CC1]

In some embodiments, B is

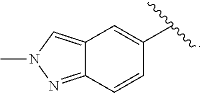 [SMILES: *c1ccc2nn(C)cc2c1]

In some embodiments, B is

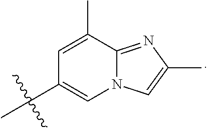 [SMILES: *c1cc(C)c2nc(C)cn2c1]

In some embodiments, B is

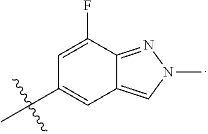 [SMILES: *c1cc(F)c2nn(C)cc2c1]

In some embodiments, B is

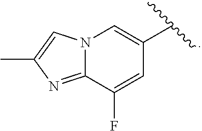 [SMILES: *c1cc(F)c2nc(C)cn2c1]

In some embodiments, B is

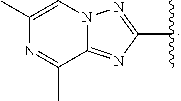 [SMILES: *c1nc2c(C)nc(C)cn2n1]

In some embodiments, B is

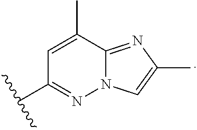 [SMILES: *c1cc(C)c2nc(C)cn2n1]

In some embodiments, B is

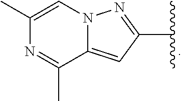 [SMILES: *c1cc2c(C)nc(C)cn2n1]

In some embodiments, B is

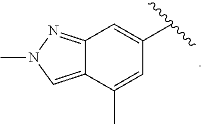 [SMILES: *c1cc(C)c2cn(C)nc2c1]

In some embodiments, B is

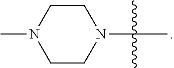 [SMILES: *N1CCN(C)CC1]

In some embodiments, B is

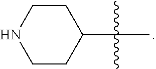 [SMILES: *C1CCNCC1]

In some embodiments, B is

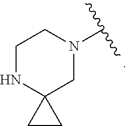 [SMILES: *N1CCNC2(CC2)C1]

In some embodiments, R¹ is C₁-C₆-alkyl. In some embodiments, R¹ is CH₃. In some embodiments, A is substituted with 0 or 1 R¹. In some embodiments, B is substituted with 0, 1, or 2 R¹.

In some embodiments, the compound of Formula (I) is selected from a compound in Table 1, or a pharmaceutically acceptable salt, solvate, hydrate, tautomer, or stereoisomer thereof.

TABLE 1
Exemplary compounds of Formula (I)

| Compound No. | Structure |
|---|---|
| 100 | 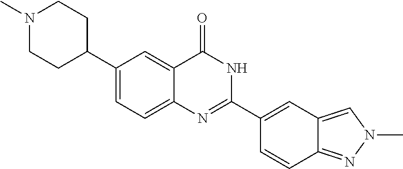 |
| 101 | 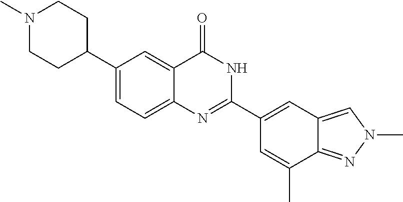 |
| 102 | 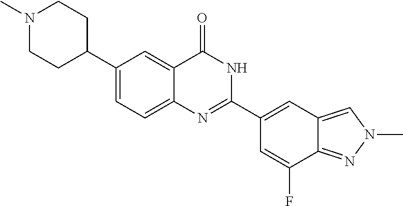 |
| 103 | 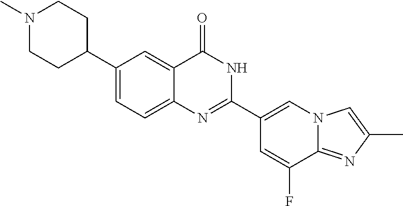 |
| 104 | 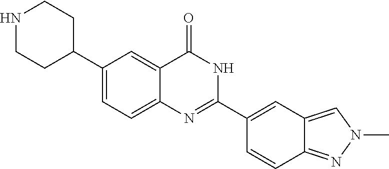 [SMILES: Cn1cc2cc(-c3nc4ccc(C5CCNCC5)cc4c(=O)[nH]3)ccc2n1] |
| 105 | 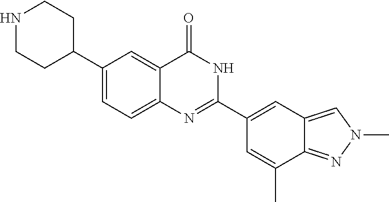 [SMILES: Cc1cc(-c2nc3ccc(C4CCNCC4)cc3c(=O)[nH]2)cc2cn(C)nc12] |
| 106 | 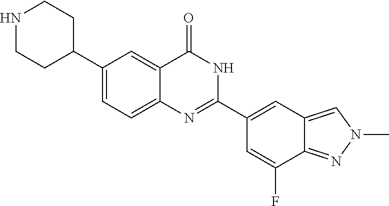 [SMILES: Cn1cc2cc(-c3nc4ccc(C5CCNCC5)cc4c(=O)[nH]3)cc(F)c2n1] |
| 107 | 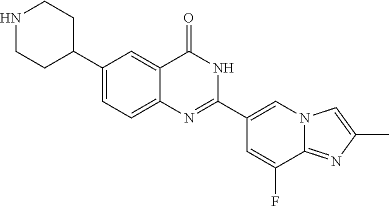 [SMILES: Cc1cn2cc(-c3nc4ccc(C5CCNCC5)cc4c(=O)[nH]3)cc(F)c2n1] |
| 108 | 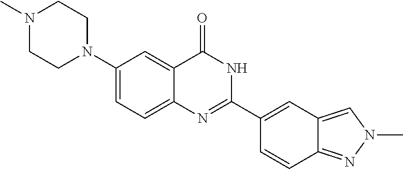 |
| 109 | 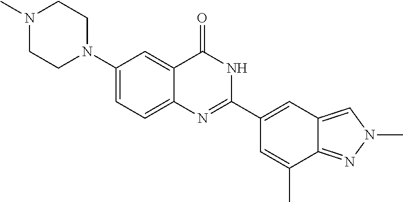 |
| 110 | 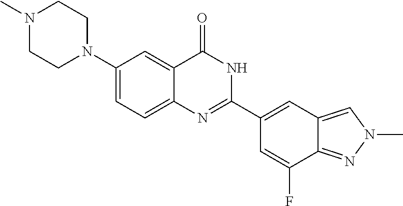 |
| 111 | 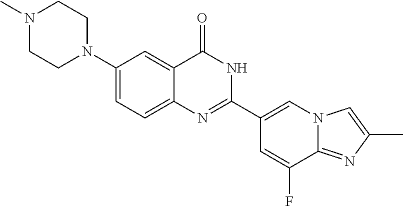 |
| 112 | 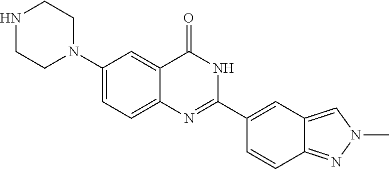 [SMILES: Cn1cc2cc(-c3nc4ccc(N5CCNCC5)cc4c(=O)[nH]3)ccc2n1] |
| 113 | 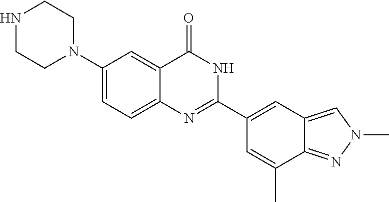 [SMILES: Cc1cc(-c2nc3ccc(N4CCNCC4)cc3c(=O)[nH]2)cc2cn(C)nc12] |
| 114 | 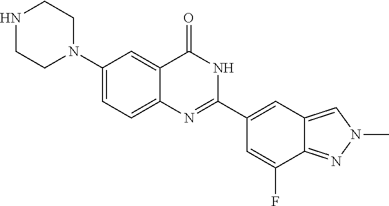 [SMILES: Cn1cc2cc(-c3nc4ccc(N5CCNCC5)cc4c(=O)[nH]3)cc(F)c2n1] |
| 115 | 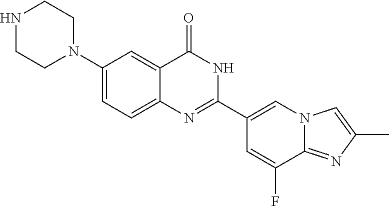 [SMILES: Cc1cn2cc(-c3nc4ccc(N5CCNCC5)cc4c(=O)[nH]3)cc(F)c2n1] |
| 116 | 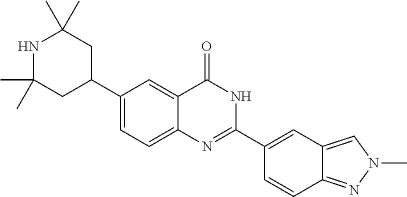 |
| 117 | 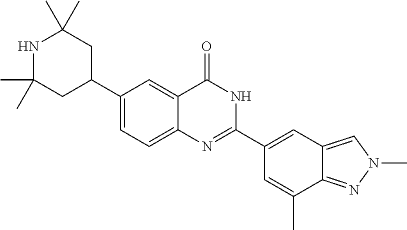 |
| 118 | 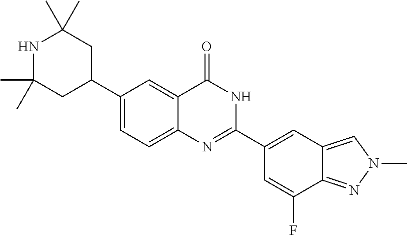 |
| 119 | 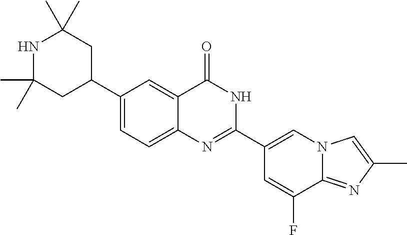 |
| 120 | 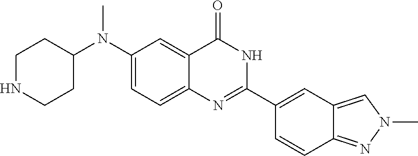 [SMILES: CN(c1ccc2nc(-c3ccc4nn(C)cc4c3)[nH]c(=O)c2c1)C1CCNCC1] |
| 121 | 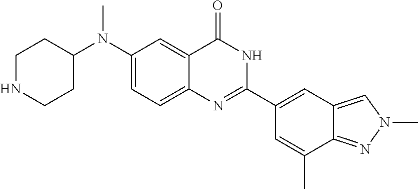 [SMILES: Cc1cc(-c2nc3ccc(N(C)C4CCNCC4)cc3c(=O)[nH]2)cc2cn(C)nc12] |
| 122 | 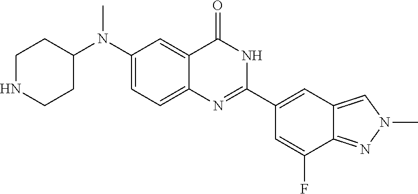 [SMILES: CN(c1ccc2nc(-c3cc(F)c4nn(C)cc4c3)[nH]c(=O)c2c1)C1CCNCC1] |
| 123 | 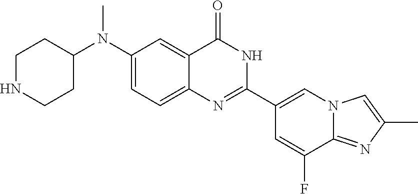 [SMILES: Cc1cn2cc(-c3nc4ccc(N(C)C5CCNCC5)cc4c(=O)[nH]3)cc(F)c2n1] |
| 124 | 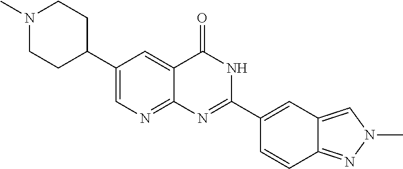 |
| 125 | 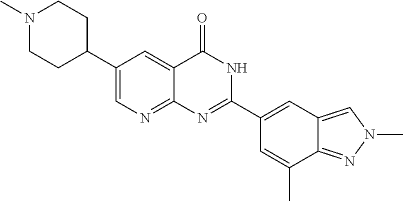 |
| 126 | 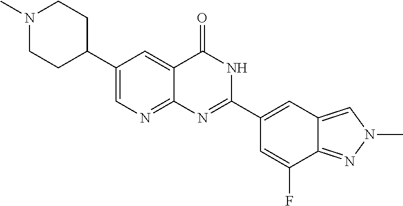 |
| 127 | 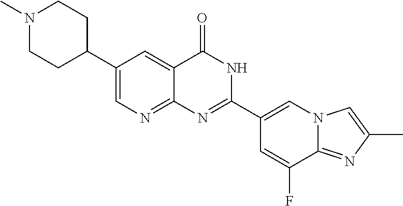 |
| 128 | 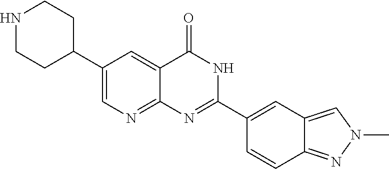 [SMILES: Cn1cc2cc(-c3nc4ncc(C5CCNCC5)cc4c(=O)[nH]3)ccc2n1] |
| 129 | 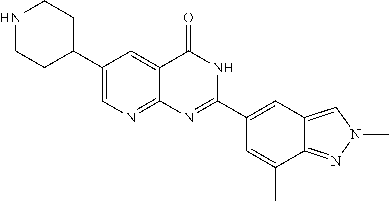 [SMILES: Cc1cc(-c2nc3ncc(C4CCNCC4)cc3c(=O)[nH]2)cc2cn(C)nc12] |
| 130 | 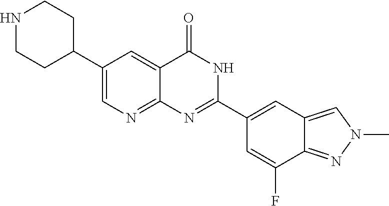 [SMILES: Cn1cc2cc(-c3nc4ncc(C5CCNCC5)cc4c(=O)[nH]3)cc(F)c2n1] |
| 131 | 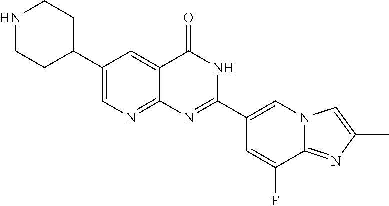 [SMILES: Cc1cn2cc(-c3nc4ncc(C5CCNCC5)cc4c(=O)[nH]3)cc(F)c2n1] |
| 132 | 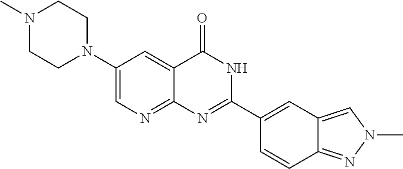 |
| 133 | 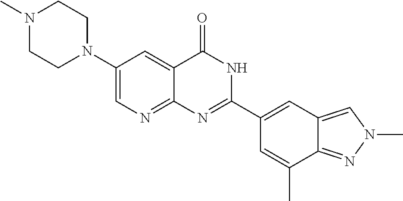 |
| 134 | 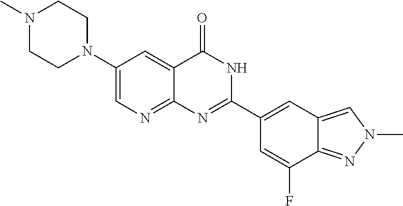 |
| 135 | 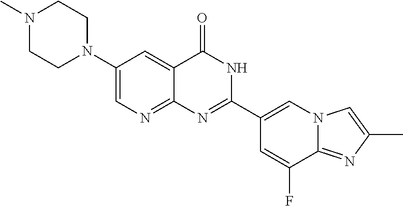 |
| 136 | 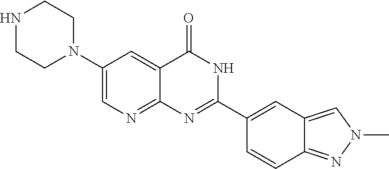 [SMILES: Cn1cc2cc(-c3nc4ncc(N5CCNCC5)cc4c(=O)[nH]3)ccc2n1] |
| 137 | 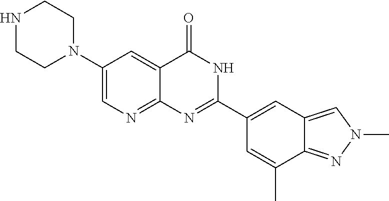 [SMILES: Cc1cc(-c2nc3ncc(N4CCNCC4)cc3c(=O)[nH]2)cc2cn(C)nc12] |
| 138 | 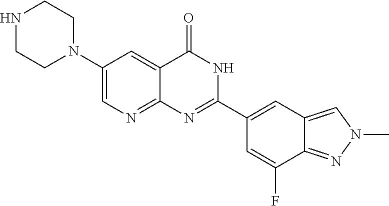 [SMILES: Cn1cc2cc(-c3nc4ncc(N5CCNCC5)cc4c(=O)[nH]3)cc(F)c2n1] |
| 139 | 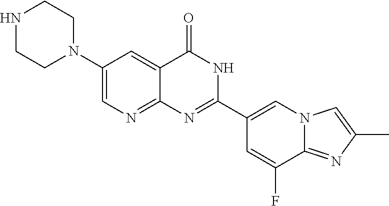 [SMILES: Cc1cn2cc(-c3nc4ncc(N5CCNCC5)cc4c(=O)[nH]3)cc(F)c2n1] |
| 140 | 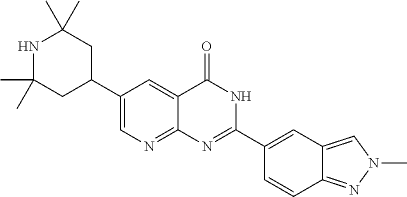 |
| 141 | 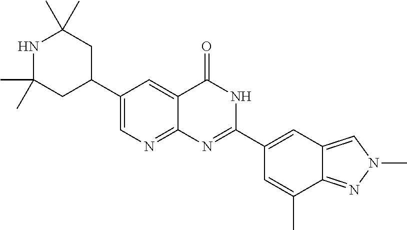 |
| 142 | 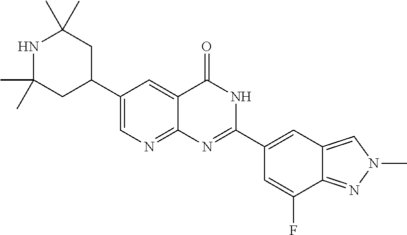 |
| 143 | 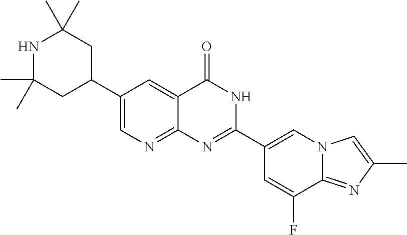 |
| 144 | 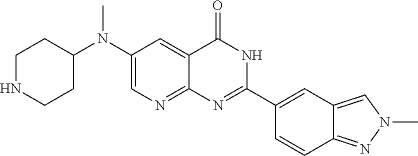 [SMILES: CN(c1cnc2nc(-c3ccc4nn(C)cc4c3)[nH]c(=O)c2c1)C1CCNCC1] |
| 145 | 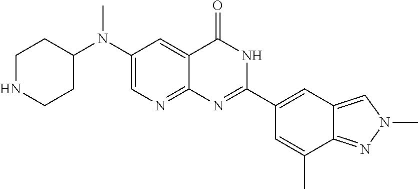 [SMILES: Cc1cc(-c2nc3ncc(N(C)C4CCNCC4)cc3c(=O)[nH]2)cc2cn(C)nc12] |
| 146 | 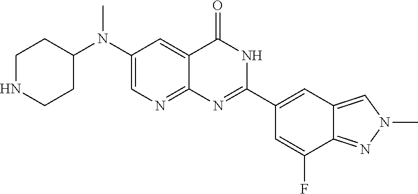 [SMILES: CN(c1cnc2nc(-c3cc(F)c4nn(C)cc4c3)[nH]c(=O)c2c1)C1CCNCC1] |
| 147 | 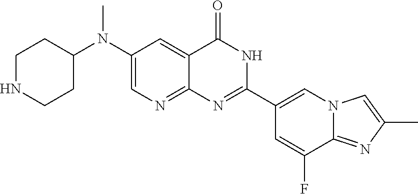 [SMILES: Cc1cn2cc(-c3nc4ncc(N(C)C5CCNCC5)cc4c(=O)[nH]3)cc(F)c2n1] |
| 165 | 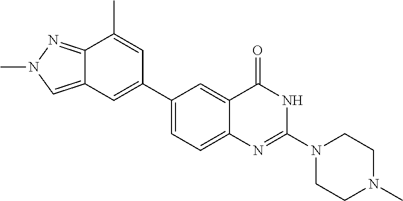 |
| 166 | 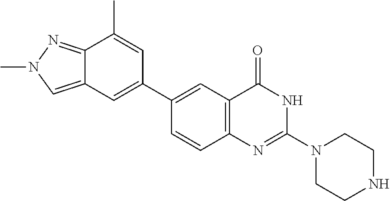 [SMILES: Cc1cc(-c2ccc3nc(N4CCNCC4)[nH]c(=O)c3c2)cc2cn(C)nc12] |
| 167 | 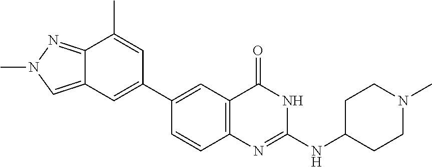 |
| 189 | 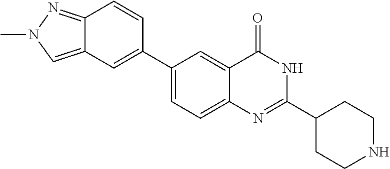 [SMILES: Cn1cc2cc(-c3ccc4nc(C5CCNCC5)[nH]c(=O)c4c3)ccc2n1] |
| 190 | 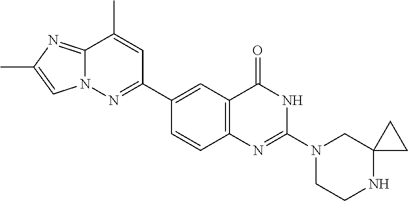 [SMILES: Cc1cn2nc(-c3ccc4nc(N5CCNC6(CC6)C5)[nH]c(=O)c4c3)cc(C)c2n1] |
| 191 | 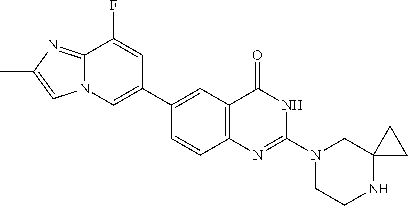 [SMILES: Cc1cn2cc(-c3ccc4nc(N5CCNC6(CC6)C5)[nH]c(=O)c4c3)cc(F)c2n1] |
| 192 | 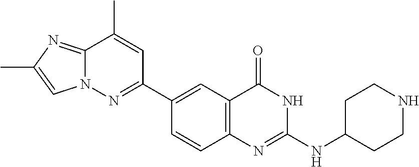 [SMILES: Cc1cn2nc(-c3ccc4nc(NC5CCNCC5)[nH]c(=O)c4c3)cc(C)c2n1] |
| 193 | 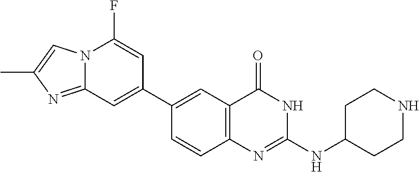 [SMILES: Cc1cn2c(F)cc(-c3ccc4nc(NC5CCNCC5)[nH]c(=O)c4c3)cc2n1] |
| 238 | 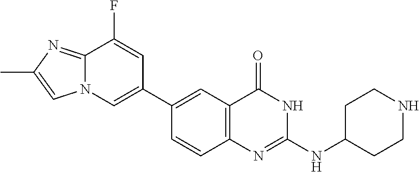 [SMILES: Cc1cn2cc(-c3ccc4nc(NC5CCNCC5)[nH]c(=O)c4c3)cc(F)c2n1] |
| 239 | 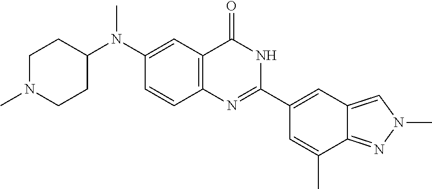 |

In some embodiments, for Formula (I), A is monocyclic heterocyclyl (e.g., N-methyl piperidinyl); B is bicyclic heteroaryl (e.g., 2-methyl-2H-indazolyl); L¹ and L² are each absent; X, W, and Z are each independently C(R³) (e.g., CH); Y is N(R^{4a}) (e.g., NH); and R² is absent. In some embodiments, the compound of Formula (I), (I-a), (I-b), and (I-c) is Compound 100, or a pharmaceutically acceptable salt, solvate, hydrate, tautomer, or stereoisomer thereof.

In some embodiments, for Formula (I), A is monocyclic heterocyclyl (e.g., N-methyl piperidinyl); B is bicyclic heteroaryl (e.g., 2,7-dimethyl-2H-indazolyl); L¹ and L² are each absent; X, W, and Z are each independently C(R³) (e.g., CH); Y is N(R^{4a}) (e.g., NH); and R² is absent. In some embodiments, the compound of Formula (I), (I-a), (I-b), and (I-c) is Compound 101, or a pharmaceutically acceptable salt, solvate, hydrate, tautomer, or stereoisomer thereof.

In some embodiments, for Formula (I), A is monocyclic heterocyclyl (e.g., N-methyl piperidinyl); B is bicyclic heteroaryl (e.g., 7-fluoro-2-methyl-2H-indazolyl); L¹ and L² are each absent; X, W, and Z are each independently C(R³) (e.g., CH); Y is N(R^{4a}) (e.g., NH); and R² is absent. In some embodiments, the compound of Formula (I), (I-a), (I-b), and (I-c) is Compound 102, or a pharmaceutically acceptable salt, solvate, hydrate, tautomer, or stereoisomer thereof.

In some embodiments, for Formula (I), A is monocyclic heterocyclyl (e.g., N-methyl piperidinyl); B is bicyclic heteroaryl (e.g., 8-fluoro-2-methylimidazo[1,2-a]pyridinyl); L¹ and L² are each absent; X, W, and Z are each independently C(R³) (e.g., CH); Y is N(R^{4a}) (e.g., NH); and R² is absent. In some embodiments, the compound of Formula (I), (I-a), (I-b), and (I-c) is Compound 103, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g., 2-methyl-2H-indazolyl); L¹ and L² are each absent; X, W, and Z are each independently C(R³) (e.g., CH); Y is N(R^{4a}) (e.g., NH); and R² is absent. In some embodiments, the compound of Formula (I), (I-a), (I-b), and (I-c) is Compound 104, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g., 2,7-dimethyl-2H-indazolyl); L¹ and L² are each absent; X, W, and Z are each independently C(R³) (e.g., CH); Y is N(R^{4a}) (e.g., NH); and R² is absent. In some embodiments, the compound of Formula (I), (I-a), (I-b), and (I-c) is Compound 105, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g., 7-fluoro-2-methyl-2H-indazolyl); L¹ and L² are each absent; X, W, and Z are each independently C(R³) (e.g., CH); Y is N(R^{4a}) (e.g., NH); and R² is absent. In some embodiments, the compound of Formula (I), (I-a), (I-b), and (I-c) is Compound 106, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g., 8-fluoro-2-methylimidazo[1,2-a]pyridinyl); L¹ and L² are each absent; X, W, and Z are each independently C(R³) (e.g., CH); Y is N(R^{4a}) (e.g., NH); and R² is absent. In some embodiments, the compound of Formula (I), (I-a), (I-b), and (I-c) is Compound 107, or a pharmaceutically acceptable salt, solvate, hydrate, tautomer, or stereoisomer thereof.

In some embodiments, for Formula (I), A is monocyclic heterocyclyl (e.g., N-methyl piperazinyl); B is bicyclic heteroaryl (e.g., 2-methyl-2H-indazolyl); L¹ and L² are each absent; X, W, and Z are each independently C(R³) (e.g., CH); Y is N(R^{4a}) (e.g., NH); and R² is absent. In some embodiments, the compound of Formula (I), (I-a), (I-b), and (I-c) is Compound 108, or a pharmaceutically acceptable salt, solvate, hydrate, tautomer, or stereoisomer thereof.

In some embodiments, for Formula (I), A is monocyclic heterocyclyl (e.g., N-methyl piperazinyl); B is bicyclic heteroaryl (e.g., 2,7-dimethyl-2H-indazolyl); L¹ and L² are each absent; X, W, and Z are each independently C(R³) (e.g., CH); Y is N(R^{4a}) (e.g., NH); and R² is absent. In some embodiments, the compound of Formula (I), (I-a), (I-b), and (I-c) is Compound 109, or a pharmaceutically acceptable salt, solvate, hydrate, tautomer, or stereoisomer thereof.

In some embodiments, for Formula (I), A is monocyclic heterocyclyl (e.g., N-methyl piperazinyl); B is bicyclic heteroaryl (e.g., 7-fluoro-2-methyl-2H-indazolyl); L¹ and L² are each absent; X, W, and Z are each independently C(R³) (e.g., CH); Y is N(R^{4a}) (e.g., NH); and R² is absent. In some embodiments, the compound of Formula (I), (I-a), (I-b), and (I-c) is Compound 110, or a pharmaceutically acceptable salt, solvate, hydrate, tautomer, or stereoisomer thereof.

In some embodiments, for Formula (I), A is monocyclic heterocyclyl (e.g., N-methyl piperazinyl); B is bicyclic heteroaryl (e.g., 8-fluoro-2-methylimidazo[1,2-a]pyridinyl); L¹ and L² are each absent; X, W, and Z are each independently C(R³) (e.g., CH); Y is N(R^{4a}) (e.g., NH); and R² is absent. In some embodiments, the compound of Formula (I), (I-a), (I-b), and (I-c) is Compound 111, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 2-methyl-2H-indazolyl); L¹ and L² are each absent; X, W, and Z are each independently C(R³) (e.g., CH); Y is N(R^{4a}) (e.g., NH); and R² is absent. In some embodiments, the compound of Formula (I), (I-a), (I-b), and (I-c) is Compound 112, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 2,7-dimethyl-2H-indazolyl); L¹ and L² are each absent; X, W, and Z are each independently C(R³) (e.g., CH); Y is N(R^{4a}) (e.g., NH); and R² is absent. In some embodiments, the compound of Formula (I), (I-a), (I-b), and (I-c) is Compound 113, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 7-fluoro-2-methyl-2H-indazolyl); L¹ and L² are each absent; X, W, and Z are each independently C(R³) (e.g., CH); Y is N(R^{4a}) (e.g., NH); and R² is absent. In some embodiments, the compound of Formula (I), (I-a), (I-b), and (I-c) is Compound 114, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 8-fluoro-2-methylimidazo[1,2-a]pyridinyl); L¹ and L² are each absent; X, W, and Z are each independently C(R³) (e.g., CH); Y is N(R^{4a}) (e.g., NH); and R² is absent. In some embodiments, the compound of Formula (I), (I-a), (I-b), and (I-c) is Compound 115, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is bicyclic heteroaryl (e.g., 2-methyl-2H-indazolyl); L¹ and L² are each absent; X, W, and Z are each independently C(R³) (e.g., CH); Y is N(R^{4a}) (e.g., NH); and R² is absent. In some embodiments, the compound of Formula (I), (I-a), (I-b), and (I-c) is Compound 116, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is bicyclic heteroaryl (e.g., 2,7-dimethyl-2H-indazolyl); L¹ and L² are each absent; X, W, and Z are each independently C(R³) (e.g., CH); Y is N(R^{4a}) (e.g., NH); and R² is absent. In some embodiments, the compound of Formula (I), (I-a), (I-b), and (I-c) is Compound 117, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is bicyclic heteroaryl (e.g., 7-fluoro-2-methyl-2H-indazolyl); L¹ and L² are each absent; X, W, and Z are each independently C(R³) (e.g., CH); Y is N(R^{4a}) (e.g., NH); and R² is absent. In some embodiments, the compound of Formula (I), (I-a), (I-b), and (I-c) is Compound 118, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is bicyclic heteroaryl (e.g., 8-fluoro-2-methylimidazo[1,2-a]pyridinyl); L¹ and L² are each absent; X, W, and Z are each independently C(R³) (e.g., CH); Y is N(R^{4a}) (e.g., NH); and R² is absent. In some embodiments, the compound of Formula (I), (I-a), (I-b), and (I-c) is Compound 119, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g., 2-methyl-2H-indazolyl); L¹ is —N(R⁸)— (e.g., —N(CH₃)—); L² is absent; X, W, and Z are each independently C(R³) (e.g., CH); Y is N(R^{4a}) (e.g., NH); and R² is absent. In some embodiments, the compound of Formula (I), (I-a), (I-b), and (I-c) is Compound 120, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g., 2,7-dimethyl-2H-indazolyl); L¹ is —N(R⁸)— (e.g., —N(CH₃)—); L² is absent; X, W, and Z are each independently C(R³) (e.g., CH); Y is N(R^{4a}) (e.g., NH); and R² is absent. In some embodiments, the compound of Formula (I), (I-a), (I-b), and (I-c) is Compound 121, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g., 7-fluoro-2-methyl-2H-indazolyl); L¹ is —N(R⁸)— (e.g., —N(CH₃)—); L² is absent; X, W, and Z are each independently C(R³) (e.g., CH); Y is N(R^{4a}) (e.g., NH); and R² is absent. In some embodiments, the compound of Formula (I), (I-a), (I-b), and (I-c) is Compound 122, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g., 8-fluoro-2-methylimidazo[1,2-a]pyridinyl); L¹ is —N(R⁸)— (e.g., —N(CH₃)—); L² is absent; X, W, and Z are each independently C(R³) (e.g., CH); Y is N(R^{4a}) (e.g., NH); and R² is absent. In some embodiments, the compound of Formula (I), (I-a), (I-b), and (I-c) is Compound 123, or a pharmaceutically acceptable salt, solvate, hydrate, tautomer, or stereoisomer thereof.

In some embodiments, for Formula (I), A is monocyclic heterocyclyl (e.g., N-methyl piperidinyl); B is bicyclic heteroaryl (e.g., 2-methyl-2H-indazolyl); L¹ and L² are each absent; X, and W are each independently C(R³) (e.g., CH); Z is N; Y is N(R^{4a}) (e.g., NH); and R² is absent. In some embodiments, the compound of Formula (I), (I-a), and (I-b) is Compound 124, or a pharmaceutically acceptable salt, solvate, hydrate, tautomer, or stereoisomer thereof.

In some embodiments, for Formula (I), A is monocyclic heterocyclyl (e.g., N-methyl piperidinyl); B is bicyclic heteroaryl (e.g., 2,7-dimethyl-2H-indazolyl); L¹ and L² are each absent; X and W are each independently C(R³) (e.g., CH); Z in N; Y is N(R^{4a}) (e.g., NH); and R² is absent. In some embodiments, the compound of Formula (I), (I-a), and (I-b) is Compound 125, or a pharmaceutically acceptable salt, solvate, hydrate, tautomer, or stereoisomer thereof.

In some embodiments, for Formula (I), A is monocyclic heterocyclyl (e.g., N-methyl piperidinyl); B is bicyclic heteroaryl (e.g., 7-fluoro-2-methyl-2H-indazolyl); L¹ and L² are each absent; X and W are each independently C(R³) (e.g., CH); Z is N; Y is N(R^{4a}) (e.g., NH); and R² is absent. In some embodiments, the compound of Formula (I), (I-a), and (I-b) is Compound 126, or a pharmaceutically acceptable salt, solvate, hydrate, tautomer, or stereoisomer thereof.

In some embodiments, for Formula (I), A is monocyclic heterocyclyl (e.g., N-methyl piperidinyl); B is bicyclic heteroaryl (e.g., 8-fluoro-2-methylimidazo[1,2-a]pyridinyl); L¹ and L² are each absent; X and W are each independently C(R³) (e.g., CH); Z is N; Y is N(R^{4a}) (e.g., NH); and R² is absent. In some embodiments, the compound of Formula (I), (I-a), and (I-b) is Compound 127, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g., 2-methyl-2H-indazolyl); L¹ and L² are each absent; X, and W are each independently C(R³) (e.g., CH); Z is N; Y is N(R^{4a}) (e.g., NH); and R² is absent. In some embodiments, the compound of Formula (I), (I-a), and (I-b) is Compound 128, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g., 2,7-dimethyl-2H-indazolyl); L¹ and L² are each absent; X and W are each independently C(R³) (e.g., CH); Z in N; Y is N(R^{4a}) (e.g., NH); and R² is absent. In some embodiments, the compound of Formula (I), (I-a), and (I-b) is Compound 129, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g., 7-fluoro-2-methyl-2H-indazolyl); L¹ and L² are each absent; X and W are each independently C(R³) (e.g., CH); Z is N; Y is N(R^{4a}) (e.g., NH); and R² is absent. In some embodiments, the compound of Formula (I), (I-a), and (I-b) is Compound 130, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g., 8-fluoro-2-methylimidazo[1,2-a]pyridinyl); L¹ and L² are each absent; X and W are each independently C(R³) (e.g., CH); Z is N; Y is N(R^{4a}) (e.g., NH); and R² is absent. In some embodiments, the compound of Formula (I), (I-a), and (I-b) is Compound 131, or a pharmaceutically acceptable salt, solvate, hydrate, tautomer, or stereoisomer thereof.

In some embodiments, for Formula (I), A is monocyclic heterocyclyl (e.g., N-methyl piperazyl); B is bicyclic heteroaryl (e.g., 2-methyl-2H-indazolyl); L¹ and L² are each absent; X, and W are each independently C(R³) (e.g., CH); Z is N; Y is N(R^{4a}) (e.g., NH); and R² is absent.

In some embodiments, the compound of Formula (I), (I-a), and (I-b) is Compound 132, or a pharmaceutically acceptable salt, solvate, hydrate, tautomer, or stereoisomer thereof. In some embodiments, for Formula (I), A is monocyclic heterocyclyl (e.g., N-methyl piperazyl); B is bicyclic heteroaryl (e.g., 2,7-dimethyl-2H-indazolyl); L¹ and L² are each absent; X and W are each independently C(R³) (e.g., CH); Z in N; Y is N(R^{4a}) (e.g., NH); and R² is absent. In some embodiments, the compound of Formula (I), (I-a), and (I-b) is Compound 133, or a pharmaceutically acceptable salt, solvate, hydrate, tautomer, or stereoisomer thereof.

In some embodiments, for Formula (I), A is monocyclic heterocyclyl (e.g., N-methyl piperazyl); B is bicyclic heteroaryl (e.g., 7-fluoro-2-methyl-2H-indazolyl); L¹ and L² are each absent; X and W are each independently C(R³) (e.g., CH); Z is N; Y is N(R^{4a}) (e.g., NH); and R² is absent. In some embodiments, the compound of Formula (I), (I-a), and (I-b) is Compound 134, or a pharmaceutically acceptable salt, solvate, hydrate, tautomer, or stereoisomer thereof.

In some embodiments, for Formula (I), A is monocyclic heterocyclyl (e.g., N-methyl piperazyl); B is bicyclic heteroaryl (e.g., 8-fluoro-2-methylimidazo[1,2-a]pyridinyl); L¹ and L² are each absent; X and W are each independently C(R³) (e.g., CH); Z is N; Y is N(R^{4a}) (e.g., NH); and R² is absent. In some embodiments, the compound of Formula (I), (I-a), and (I-b) is Compound 135, or a pharmaceutically acceptable salt, solvate, hydrate, tautomer, or stereoisomer thereof.

In some embodiments, for Formula (I), A is monocyclic heterocyclyl (e.g., piperazyl); B is bicyclic heteroaryl (e.g., 2-methyl-2H-indazolyl); L¹ and L² are each absent; X and W are each independently C(R³) (e.g., CH); Z is N; Y is N(R^{4a}) (e.g., NH); and R² is absent. In some embodiments, the compound of Formula (I), (I-a), and (I-b) is Compound 136, or a pharmaceutically acceptable salt, solvate, hydrate, tautomer, or stereoisomer thereof.

In some embodiments, for Formula (I), A is monocyclic heterocyclyl (e.g., piperazyl); B is bicyclic heteroaryl (e.g., 2,7-dimethyl-2H-indazolyl); L¹ and L² are each absent; X and W are each independently C(R³) (e.g., CH); Z in N; Y is N(R^{4a}) (e.g., NH); and R² is absent. In some embodiments, the compound of Formula (I), (I-a), and (I-b) is Compound 137, or a pharmaceutically acceptable salt, solvate, hydrate, tautomer, or stereoisomer thereof.

In some embodiments, for Formula (I), A is monocyclic heterocyclyl (e.g., piperazyl); B is bicyclic heteroaryl (e.g., 7-fluoro-2-methyl-2H-indazolyl); L¹ and L² are each absent; X and W are each independently C(R³) (e.g., CH); Z is N; Y is N(R^{4a}) (e.g., NH); and R² is absent. In some embodiments, the compound of Formula (I), (I-a), and (I-b) is Compound 138, or a pharmaceutically acceptable salt, solvate, hydrate, tautomer, or stereoisomer thereof.

In some embodiments, for Formula (I), A is monocyclic heterocyclyl (e.g., piperazyl); B is bicyclic heteroaryl (e.g., 8-fluoro-2-methylimidazo[1,2-a]pyridinyl); L¹ and L² are each absent; X and W are each independently C(R³) (e.g., CH); Z is N; Y is N(R^{4a}) (e.g., NH); and R² is absent. In some embodiments, the compound of Formula (I), (I-a), and (I-b) is Compound 139, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is bicyclic heteroaryl (e.g., 2-methyl-2H-indazolyl); L¹ and L² are each absent; X and W are each independently C(R³) (e.g., CH); Z is N; Y is N(R^{4a}) (e.g., NH); and R² is absent. In some embodiments, the compound of Formula (I), (I-a), and (I-b) is Compound 140, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is bicyclic heteroaryl (e.g., 2,7-dimethyl-2H-indazolyl); L¹ and L² are each absent; X and W are each independently C(R³) (e.g., CH); Z in N; Y is N(R^{4a}) (e.g., NH); and R² is absent. In some embodiments, the compound of Formula (I), (I-a), and (I-b) is Compound 141, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is bicyclic heteroaryl (e.g., 7-fluoro-2-methyl-2H-indazolyl); L¹ and L² are each absent; X and W are each independently C(R³) (e.g., CH); Z is N; Y is N(R^{4a}) (e.g., NH); and R² is absent. In some embodiments, the compound of Formula (I), (I-a), and (I-b) is Compound 142, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is bicyclic heteroaryl (e.g., 8-fluoro-2-methylimidazo[1,2-a]pyridinyl); L¹ and L² are each absent; X and W are each independently C(R³) (e.g., CH); Z is N; Y is N(R^{4a}) (e.g., NH); and R² is absent. In some embodiments, the compound of Formula (I), (I-a), and (I-b) is Compound 143, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g., 2-methyl-2H-indazolyl); L¹ is —N(R⁸)— (e.g., —N(CH₃)—); L² is absent; X and W are each independently C(R³) (e.g., CH); Z is N; Y is N(R^{4a}) (e.g., NH); and R² is absent. In some embodiments, the compound of Formula (I), (I-a), and (I-b) is Compound 144, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g., 2,7-dimethyl-2H-indazolyl); L¹ is —N(R⁸)— (e.g., —N(CH₃)—); L² is absent; X and W are each independently C(R³) (e.g., CH); Z in N; Y is N(R^{4a}) (e.g., NH); and R² is absent. In some embodiments, the compound of Formula (I), (I-a), and (I-b) is Compound 145, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g., 7-fluoro-2-methyl-2H-indazolyl); L¹ is —N(R⁸)— (e.g., —N(CH₃)—); L² is absent; X and W are each independently C(R³) (e.g., CH); Z is N; Y is N(R^{4a}) (e.g., NH); and R² is absent. In some embodiments, the compound of Formula (I), (I-a), and (I-b) is Compound 146, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g., 8-fluoro-2-methylimidazo[1,2-a]pyridinyl); L¹ is —N(R⁸)— (e.g., —N(CH₃)—); L² is absent; X and W are each independently C(R³) (e.g., CH); Z is N; Y is N(R^{4a}) (e.g., NH); and R² is absent. In some embodiments, the compound of Formula (I), (I-a), and (I-b) is Compound 147, or a pharmaceutically acceptable salt, solvate, hydrate, tautomer, or stereoisomer thereof.

In some embodiments, for Formula (I), A is bicyclic heterocyclyl (e.g., 2,7-dimethyl-2H-indazolyl); B is monocyclic heteroaryl (e.g., N-methyl piperazyl); L¹ and L² are each absent; X, W, and Z are each independently C(R³) (e.g., CH); Y is N(R^{4a}) (e.g., NH); and R² is absent. In some embodiments, the compound of Formula (I), (I-a), (I-b), and (I-c) is Compound 165, or a pharmaceutically acceptable salt, solvate, hydrate, tautomer, or stereoisomer thereof.

In some embodiments, for Formula (I), A is bicyclic heterocyclyl (e.g., 2,7-dimethyl-2H-indazolyl); B is monocyclic heteroaryl (e.g., piperazyl); L¹ and L² are each absent; X, W, and Z are each independently C(R³) (e.g., CH); Y is N(R^{4a}) (e.g., NH); and R² is absent. In some embodiments, the compound of Formula (I), (I-a), (I-b), and (I-c) is Compound 166, or a pharmaceutically acceptable salt, solvate, hydrate, tautomer, or stereoisomer thereof.

In some embodiments, for Formula (I), A is bicyclic heterocyclyl (e.g., 2,7-dimethyl-2H-indazolyl); B is monocyclic heteroaryl (e.g., N-methyl piperidinyl); L¹ is absent; L² is —N(R⁸)— (e.g., —N(H)—); X, W, and Z are each independently C(R³) (e.g., CH); Y is N(R^{4a}) (e.g., NH); and R² is absent. In some embodiments, the compound of Formula (I), (I-a), (I-b), and (I-c) is Compound 167, or a pharmaceutically acceptable salt, solvate, hydrate, tautomer, or stereoisomer thereof.

In some embodiments, for Formula (I), A is bicyclic heterocyclyl (e.g., 2-methyl-2H-indazolyl); B is monocyclic heteroaryl (e.g., piperidinyl); L¹ and L² are each absent; X, W, and Z are each independently C(R³) (e.g., CH); Y is N(R^{4a}) (e.g., NH); and R² is absent. In some embodiments, the compound of Formula (I), (I-a), (I-b), and (I-c) is Compound 189, or a pharmaceutically acceptable salt, solvate, hydrate, tautomer, or stereoisomer thereof.

In some embodiments, for Formula (I), A is bicyclic heterocyclyl (e.g., 2,8-dimethylimidazo[1,2-b]pyridazyl); B is monocyclic heteroaryl (e.g., 4,7-diazaspiro[2.5]octanyl); L¹ and L² are each absent; X, W, and Z are each independently C(R³) (e.g., CH); Y is N(R^{4a}) (e.g., NH); and R² is absent. In some embodiments, the compound of Formula (I), (I-a), (I-b), and (I-c) is Compound 190, or a pharmaceutically acceptable salt, solvate, hydrate, tautomer, or stereoisomer thereof.

In some embodiments, for Formula (I), A is bicyclic heterocyclyl (e.g., 8-fluoro-2-methylimidazo[1,2-a]pyridinyl); B is monocyclic heteroaryl (e.g., 4,7-diazaspiro[2.5]octanyl); L¹ and L² are each absent; X, W, and Z are each independently C(R³) (e.g., CH); Y is N(R^{4a}) (e.g., NH); and R² is absent. In some embodiments, the compound of Formula (I), (I-a), (I-b), and (I-c) is Compound 191, or a pharmaceutically acceptable salt, solvate, hydrate, tautomer, or stereoisomer thereof.

In some embodiments, for Formula (I), A is bicyclic heterocyclyl (e.g., 2,8-dimethylimidazo[1,2-b]pyridazyl); B is monocyclic heteroaryl (e.g., piperidinyl); L¹ is absent; L² is —N(R⁸)— (e.g., —N(H)—); X, W, and Z are each independently C(R³) (e.g., CH); Y is N(R^{4a}) (e.g., NH); and R² is absent. In some embodiments, the compound of Formula (I), (I-a), (I-b), and (I-c) is Compound 192, or a pharmaceutically acceptable salt, solvate, hydrate, tautomer, or stereoisomer thereof.

In some embodiments, for Formula (I), A is bicyclic heterocyclyl (e.g., 5-fluoro methylimidazo[1,2-a]pyridinyl); B is monocyclic heteroaryl (e.g., piperidinyl); L¹ is absent; L² is —N(R⁸)— (e.g., —N(H)—); X, W, and Z are each independently C(R³) (e.g., CH); Y is N(R^{4a}) (e.g., NH); and R² is absent. In some embodiments, the compound of Formula (I), (I-a), (I-b), and (I-c) is Compound 193, or a pharmaceutically acceptable salt, solvate, hydrate, tautomer, or stereoisomer thereof.

In some embodiments, for Formula (I), A is bicyclic heterocyclyl (e.g., 8-fluoro-2-methylimidazo[1,2-a]pyridinyl); B is monocyclic heteroaryl (e.g., piperidinyl); L¹ is absent; L² is —N(R⁸)— (e.g., —N(H)—); X, W, and Z are each independently C(R³) (e.g., CH); Y is N(R^{4a}) (e.g., NH); and R² is absent. In some embodiments, the compound of Formula (I), (I-a), (I-b), and (I-c) is Compound 238, or a pharmaceutically acceptable salt, solvate, hydrate, tautomer, or stereoisomer thereof.

In some embodiments, for Formula (I), A is monocyclic heterocyclyl (e.g., N-methyl piperidinyl); B is bicyclic heteroaryl (e.g., 2,7-dimethyl-2H-indazolyl); L¹ is —N(R⁸)— (e.g., —N(CH₃)—); L² is absent; X, W, and Z are each independently C(R³) (e.g., CH); Y is N(R^{4a}) (e.g., NH); and R² is absent. In some embodiments, the compound of Formula (I), (I-a), (I-b), and (I-c) is Compound 239, or a pharmaceutically acceptable salt, solvate, hydrate, tautomer, or stereoisomer thereof.

As generally described for Formula (III), Y may be N, C, or C(R^{4b}), wherein the dashed lines in the ring comprising Y may be single or double bonds as valency permits. In some embodiments, Y is N or C. In some embodiments, Y is N (e.g., N). In some embodiments, Y is C.

In some embodiments, Z is C(R³) and Y is N. In some embodiments, Z is CH and Y is N. In some embodiments, X is C(R³) and Y is N. In some embodiments, X is CH and Y is N. In some embodiments, Z is C(R³) and Y is N. In some embodiments, Z is CH and Y is N. In some embodiments, Z and X are independently C(R³) and Y is N. In some embodiments, Z and X are independently CH and Y is N. In some embodiments, X and Z are independently C(R³) and Y is N. In some embodiments, X and Z are independently C(R³) and Y is N. In some embodiments, X and Z are independently CH and Y is N.

In some embodiments, the compound of Formula (III) is a compound of Formula (III-a):

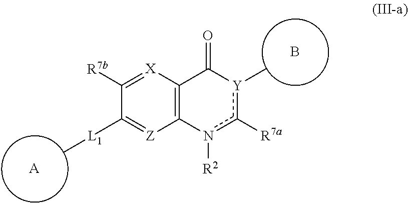

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L¹ is absent, C₁-C₆-alkylene, C₁-C₆-heteroalkylene, —O—, —C(O)—, —N(R⁸)—, —N(R⁸)C(O)—, or —C(O)N(R⁸)—, wherein each alkylene and heteroalkylene is optionally substituted with one or more R⁹; each of X and Z is independently C(R³) or N; Y is N, C, or C(R^{4b}), wherein the dashed lines in the ring comprising Y may be single or double bonds as valency permits; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, NR^{B}C(O)R^{D}, NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁵; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵; R² is absent, hydrogen, or C₁-C₆-alkyl; R³ is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —C(O)R^{D}, or —C(O)OR^{D}; R^{4b} is hydrogen, C₁-C₆-alkyl, C₁-C₆-heteroalkyl, or C₁-C₆-haloalkyl; each R⁵ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁶; each R⁶ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; R^{7a} is hydrogen, C₁-C₆-alkyl, C₁-C₆-heteroalkyl, C₁-C₆-haloalkyl, halo, cyano, oxo, or —OR^{A}; R^{7b} is hydrogen, C₁-C₆-alkyl, C₁-C₆-heteroalkyl, C₁-C₆-haloalkyl, halo, cyano, or -OR^{A}; each R⁸ is independently hydrogen, C₁-C₆-alkyl, or C₁-C₆-haloalkyl; each R⁹ is independently C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each R^{B} and R^{C} is independently hydrogen, C₁-C₆ alkyl, C₁-C₆ heteroalkyl, cycloalkyl, heterocyclyl, —OR^{A}; or R^{B} and R^{C} together with the atom to which they are attached form a 3-7-membered heterocyclyl ring optionally substituted with one or more R¹⁰; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R¹⁰ is independently C₁-C₆-alkyl or halo; and x is 0, 1, or 2.

In some embodiments, A is heterocyclyl optionally substituted with one or more R¹. In some embodiments, A is monocyclic nitrogen-containing heterocyclyl. In some embodiments, A is optionally substituted piperidinyl. In some embodiments, A is

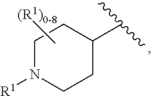

wherein each R¹ is independently hydrogen or C₁-C₆-alkyl. In some embodiments, A is

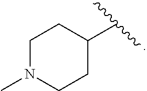 [SMILES: *C1CCN(C)CC1]

In some embodiments, A is

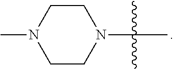 [SMILES: *N1CCN(C)CC1]

In some embodiments, A is

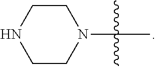 [SMILES: *N1CCNCC1]

In some embodiments, A is heteroaryl optionally substituted with one or more R¹. In some embodiments, A is bicyclic nitrogen-containing heteroaryl. In some embodiments, A is optionally substituted indazolyl. In some embodiments, A is optionally substituted imidazo[1,2-a]pyridinyl. In some embodiments, A is

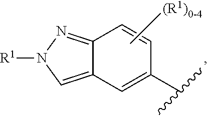

wherein each R¹ is as defined herein. In some embodiments, A is

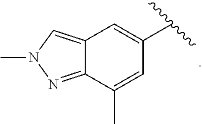 [SMILES: *c1cc(C)c2nn(C)cc2c1]

In some embodiments, A is

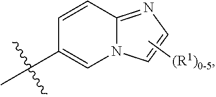

wherein each R¹ is as defined herein. In some embodiments, A is

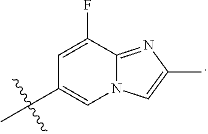 [SMILES: *c1cc(F)c2nc(C)cn2c1]

In some embodiments, A is

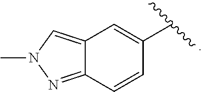 [SMILES: *c1ccc2nn(C)cc2c1]

In some embodiments, B is heteroaryl optionally substituted with one or more R¹. In some embodiments, B is bicyclic nitrogen-containing heteroaryl. In some embodiments, B is optionally substituted indazolyl. In some embodiments, B is selected from

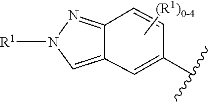

In some embodiments, B is selected from

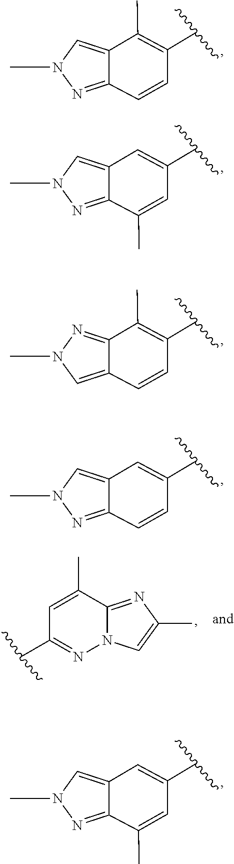

In some embodiments, B is

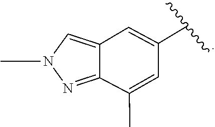 [SMILES: *c1cc(C)c2nn(C)cc2c1]

In some embodiments, B is

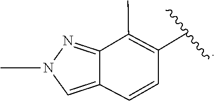 [SMILES: *c1ccc2cn(C)nc2c1C]

In some embodiments, B is

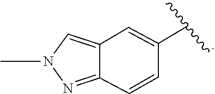 [SMILES: *c1ccc2nn(C)cc2c1]

In some embodiments, B is heterocyclyl optionally substituted with one or more R¹. In some embodiments, B is monocyclic nitrogen-containing heterocyclyl. In some embodiments, B is optionally substituted piperazinyl. In some embodiments, B is

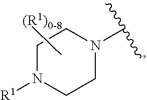

wherein R¹ is as defined herein. In some embodiments, B is

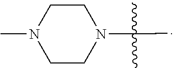 [SMILES: *N1CCN(C)CC1]

In some embodiments, B is

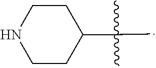 [SMILES: *C1CCNCC1]

As generally described, Y may be N, C, or C(R^{4b}), wherein the dashed lines in the ring comprising Y may be single or double bonds as valency permits. In some embodiments, Y is N. In some embodiments, Y is C. In some embodiments, Y is C(R^{4b}) (e.g., CH).

In some embodiments, L¹ is absent or N(CH₃). In some embodiments, L¹ is absent. In some embodiments, L¹ is N(CH₃).

In some embodiments, each of R^{7a} and R^{7b} is independently hydrogen.

In some embodiments, R² is absent. In some embodiments, R⁷ is hydrogen.

In some embodiments, R¹ is C₁-C₆-alkyl. In some embodiments, R¹ is CH₃. In some embodiments, A is substituted with 0 or 1 In some embodiments, B is substituted with 0, 1, or 2 R¹.

In some embodiments, the compound of Formula (III) is a compound of Formula (III-b):

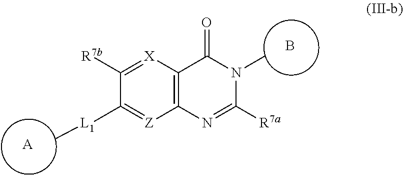

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L¹ is absent, C₁-C₆-alkylene, C₁-C₆-heteroalkylene, —O—, —C(O)—, —N(R⁸)—, —N(R⁸)C(O)—, or —C(O)N(R⁸)—, wherein each alkylene and heteroalkylene is optionally substituted with one or more R⁹; each of X and Z is independently C(R³) or N; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁵; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵; R³ is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —C(O)R^{D}, or —C(O)OR^{D}; each R⁵ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁶; each R⁶ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; R^{7a} is hydrogen, C₁-C₆-alkyl, C₁-C₆-heteroalkyl, C₁-C₆-haloalkyl, halo, cyano, oxo, or —OR^{A}; R^{7b} is hydrogen, C₁-C₆-alkyl, C₁-C₆-heteroalkyl, C₁-C₆-haloalkyl, halo, cyano, or —OR^{A}; each R⁸ is independently hydrogen, C₁-C₆-alkyl, or C₁-C₆-haloalkyl; each R⁹ is independently C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each R^{B} and R^{C} is independently hydrogen, C₁-C₆ alkyl, C₁-C₆ heteroalkyl, cycloalkyl, heterocyclyl, —OR^{A}; or R^{B} and R^{C} together with the atom to which they are attached form a 3-7-membered heterocyclyl ring optionally substituted with one or more R¹⁰; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each le° is independently C₁-C₆-alkyl or halo; and x is 0, 1, or 2.

In some embodiments, A is heterocyclyl optionally substituted with one or more R¹. In some embodiments, A is monocyclic nitrogen-containing heterocyclyl. In some embodiments, A is optionally substituted piperidinyl. In some embodiments, A is

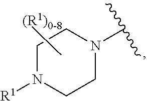

wherein each R¹ is independently hydrogen or C₁-C₆-alkyl. In some embodiments, A is

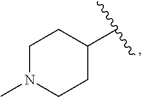 [SMILES: *C1CCN(C)CC1]

In some embodiments, A is

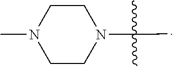 [SMILES: *N1CCN(C)CC1]

In some embodiments, A is

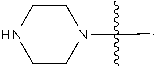 [SMILES: *N1CCNCC1]

In some embodiments, A is heteroaryl optionally substituted with one or more R¹. In some embodiments, A is bicyclic nitrogen-containing heteroaryl. In some embodiments, A is optionally substituted indazolyl. In some embodiments, A is optionally substituted imidazo[1,2-a]pyridinyl. In some embodiments, A is

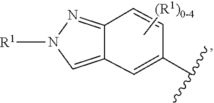

wherein each R¹ is as defined herein. In some embodiments, A is

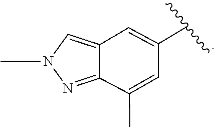 [SMILES: *c1cc(C)c2nn(C)cc2c1]

In some embodiments, A is

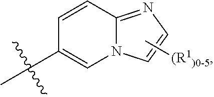

wherein each R¹ is as defined herein. In some embodiments, A is

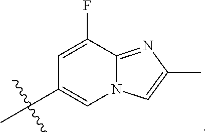 [SMILES: *c1cc(F)c2nc(C)cn2c1]

In some embodiments, A is

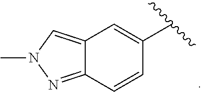 [SMILES: *c1ccc2nn(C)cc2c1]

In some embodiments, B is heteroaryl optionally substituted with one or more R¹. In some embodiments, B is bicyclic nitrogen-containing heteroaryl. In some embodiments, B is optionally substituted indazolyl. In some embodiments, B is selected from

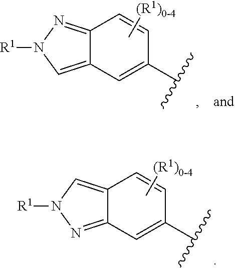

In some embodiments, B is selected from

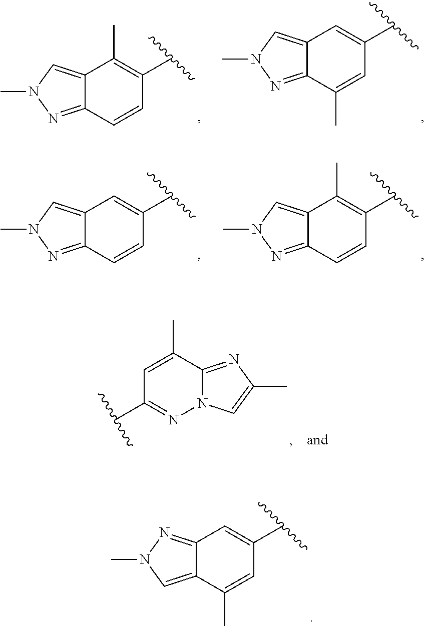

In some embodiments, B is

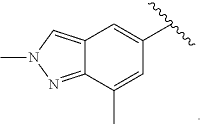 [SMILES: *c1cc(C)c2nn(C)cc2c1]

In some embodiments, B is

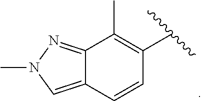 [SMILES: *c1ccc2cn(C)nc2c1C]

In some embodiments, B is heterocyclyl optionally substituted with one or more R¹. In some embodiments, B is monocyclic nitrogen-containing heterocyclyl. In some embodiments, B is optionally substituted piperazinyl. In some embodiments, B is

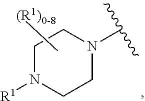

wherein R¹ is as defined herein. In some embodiments, B is

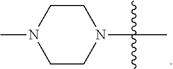 [SMILES: *N1CCN(C)CC1]

In some embodiments, B is

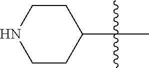 [SMILES: *C1CCNCC1]

In some embodiments, L¹ is absent.

In some embodiments, each of X and Z may independently be N or C(R³). In some embodiments, X is C(R³) (e.g., CH). In some embodiments, X is N. In some embodiments, Z is C(R³) (e.g., CH). In some embodiments, Z is N. In some embodiments, each of X and Z is independently C(R³) (e.g., CH). In some embodiments, each of X and Z is independently C(R³) (e.g., CH).

In some embodiments, R¹ is C₁-C₆-alkyl. In some embodiments, R¹ is CH₃. In some embodiments, A is substituted with 0 or 1 In some embodiments, B is substituted with 0, 1, or 2 R¹.

In some embodiments, each of R^{7a} and R^{7b} is independently hydrogen. In some embodiments, the compound of Formula (III) is a compound of Formula (III-c):

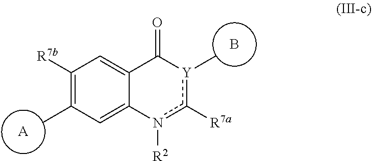

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; Y is N, C, or C(R^{4b}), wherein the dashed lines in the ring comprising Y may be single or double bonds as valency permits; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —^{NO}₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁵; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵; R² is absent, hydrogen, or C₁-C₆-alkyl; R^{4a} is hydrogen, C₁-C₆-alkyl, C₁-C₆-heteroalkyl, or C₁-C₆-haloalkyl; each R⁵ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁶; each R⁶ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; R^{7a} is hydrogen, C₁-C₆-alkyl, C₁-C₆-heteroalkyl, C₁-C₆-haloalkyl, halo, cyano, oxo, or —OR^{A}; R^{7b} is hydrogen, C₁-C₆-alkyl, C₁-C₆-heteroalkyl, C₁-C₆-haloalkyl, halo, cyano, or —OR^{A}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each R^{B} and R^{C} is independently hydrogen, C₁-C₆ alkyl, C₁-C₆ heteroalkyl, cycloalkyl, heterocyclyl, —OR^{A}; or R^{B} and R^{C} together with the atom to which they are attached form a 3-7-membered heterocyclyl ring optionally substituted with one or more R¹⁰; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each le° is independently C₁-C₆-alkyl or halo; and x is 0, 1, or 2.

In some embodiments, A is heterocyclyl optionally substituted with one or more R¹. In some embodiments, A is monocyclic nitrogen-containing heterocyclyl. In some embodiments, A is optionally substituted piperidinyl. In some embodiments, A is

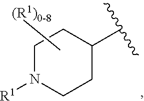

wherein each R¹ is independently hydrogen or C₁-C₆-alkyl. In some embodiments, A is

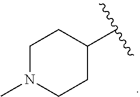 [SMILES: *C1CCN(C)CC1]

In some embodiments, A is

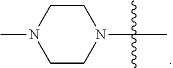 [SMILES: *N1CCN(C)CC1]

In some embodiments, A is

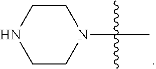 [SMILES: *N1CCNCC1]

In some embodiments, A is heteroaryl optionally substituted with one or more R¹. In some embodiments, A is bicyclic nitrogen-containing heteroaryl. In some embodiments, A is optionally substituted indazolyl. In some embodiments, A is optionally substituted imidazo[1,2-a]pyridinyl. In some embodiments, A is

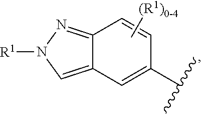

wherein each R¹ is as defined herein. In some embodiments, A is

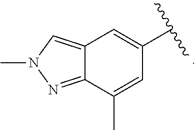 [SMILES: *c1cc(C)c2nn(C)cc2c1]

In some embodiments, A is

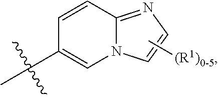

wherein each R¹ is as defined herein. In some embodiments, A is

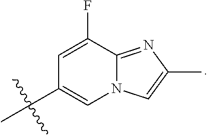 [SMILES: *c1cc(F)c2nc(C)cn2c1]

In some embodiments, A is

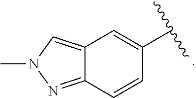 [SMILES: *c1ccc2nn(C)cc2c1]

In some embodiments, B is heteroaryl optionally substituted with one or more R¹. In some embodiments, B is bicyclic nitrogen-containing heteroaryl. In some embodiments, B is optionally substituted indazolyl. In some embodiments, B is selected from

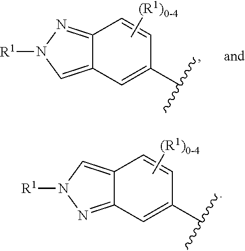

In some embodiments, B is selected from

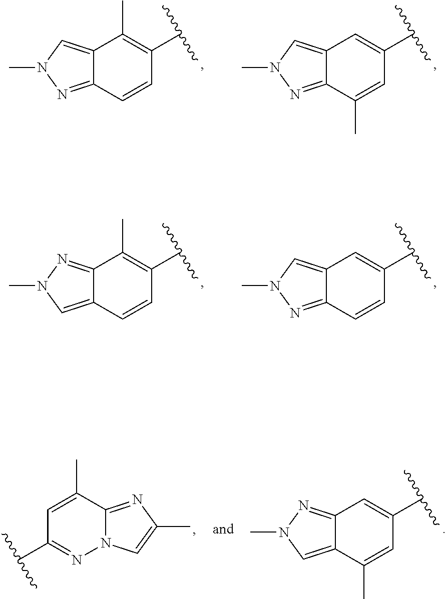 [SMILES: *c1ccc2nn(C)cc2c1C ; *c1cc(C)c2nn(C)cc2c1 ; *c1ccc2cn(C)nc2c1C ; *c1ccc2nn(C)cc2c1 ; *c1cc(C)c2nc(C)cn2n1 ; *c1cc(C)c2cn(C)nc2c1]

In some embodiments, B is

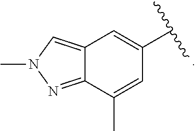 [SMILES: *c1cc(C)c2nn(C)cc2c1]

In some embodiments, B is

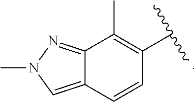 [SMILES: *c1ccc2cn(C)nc2c1C]

In some embodiments, B is

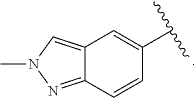 [SMILES: *c1ccc2nn(C)cc2c1]

In some embodiments, Y is N, wherein the dashed lines in the ring comprising Y may be single or double bonds as valency permits. In some embodiments, Y is N or C(R^{4b}). In some embodiments, Y is N (e.g., N). In some embodiments, Y is C(R^{4b}) (e.g., CH).

In some embodiments, L¹ is absent.

In some embodiments, R² is absent.

In some embodiments, each of R^{7a} and R^{7b} is independently hydrogen.

In some embodiments, R¹ is C₁-C₆-alkyl. In some embodiments, R¹ is CH₃. In some embodiments, A is substituted with 0 or 1 R¹. In some embodiments, B is substituted with 0, 1, or 2 R¹.

In some embodiments, the compound of Formula (III) is selected from a compound in Table 3, or a pharmaceutically acceptable salt, solvate, hydrate, tautomer, or stereoisomer thereof.

TABLE 3
Exemplary compounds of Formula (III)

| Compound | |
|---|---|
| No. | Structure |
| 152 | 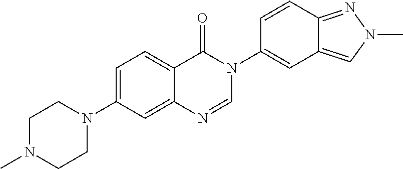 |
| 153 | 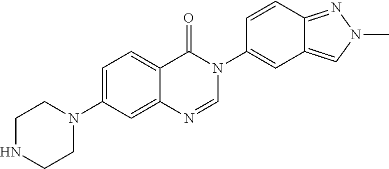 [SMILES: Cn1cc2cc(-n3cnc4cc(N5CCNCC5)ccc4c3=O)ccc2n1] |
| 156 | 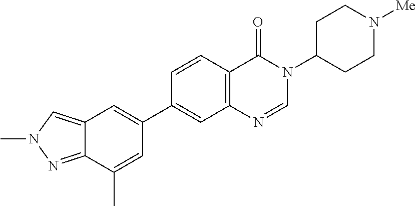 [SMILES: Cc1cc(-c2ccc3c(=O)n(C4CCN(C)CC4)cnc3c2)cc2cn(C)nc12] |
| 157 | 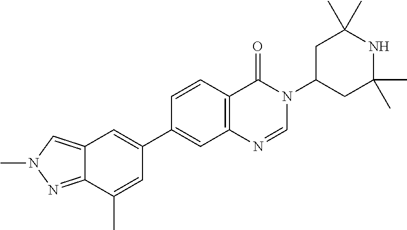 |
| 158 | 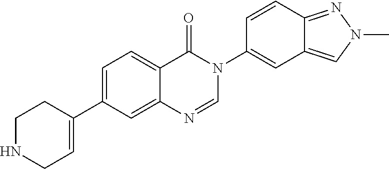 [SMILES: Cn1cc2cc(-n3cnc4cc(C5=CCNCC5)ccc4c3=O)ccc2n1] |
| 159 | 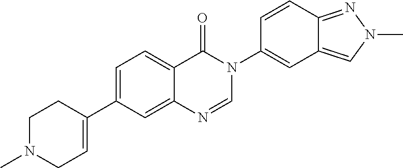 |
| 160 | 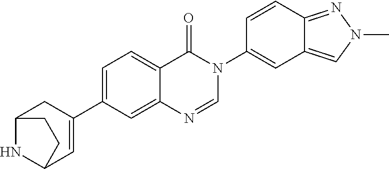 [SMILES: Cn1cc2cc(-n3cnc4cc(C5=CC6CCC(C5)N6)ccc4c3=O)ccc2n1] |
| 161 | 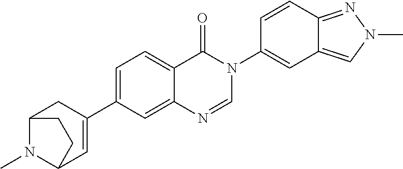 |
| 162 | 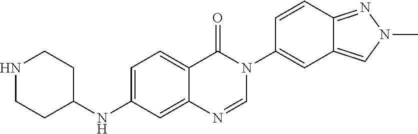 [SMILES: Cn1cc2cc(-n3cnc4cc(NC5CCNCC5)ccc4c3=O)ccc2n1] |
| 163 | 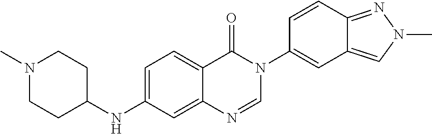 |
| 172 | 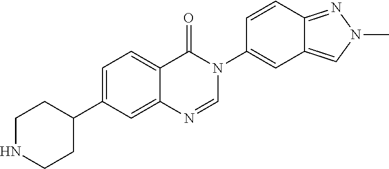 [SMILES: Cn1cc2cc(-n3cnc4cc(C5CCNCC5)ccc4c3=O)ccc2n1] |
| 173 | 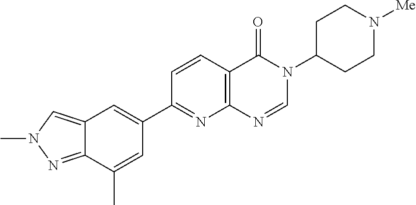 [SMILES: Cc1cc(-c2ccc3c(=O)n(C4CCN(C)CC4)cnc3n2)cc2cn(C)nc12] |
| 174 | 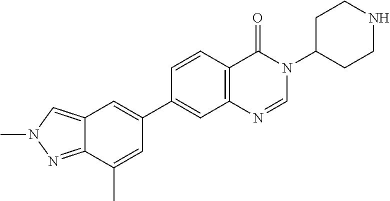 [SMILES: Cc1cc(-c2ccc3c(=O)n(C4CCNCC4)cnc3c2)cc2cn(C)nc12] |
| 175 | 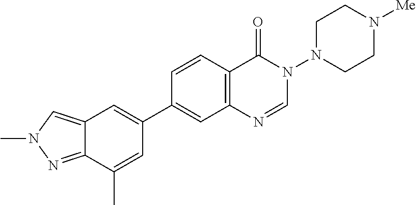 [SMILES: Cc1cc(-c2ccc3c(=O)n(N4CCN(C)CC4)cnc3c2)cc2cn(C)nc12] |
| 176 | 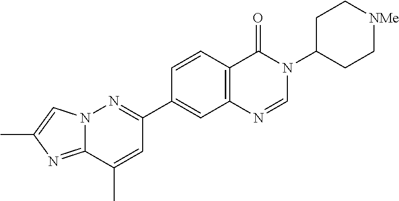 [SMILES: Cc1cn2nc(-c3ccc4c(=O)n(C5CCN(C)CC5)cnc4c3)cc(C)c2n1] |
| 177 | 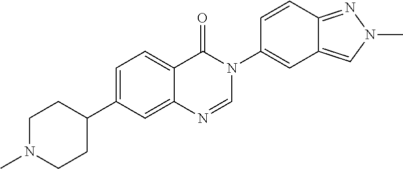 |
| 178 | 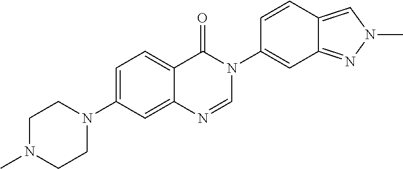 |
| 179 | 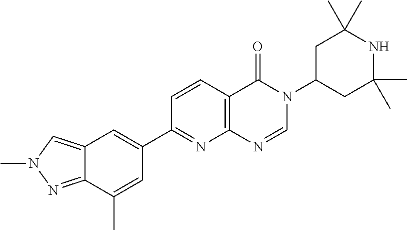 |
| 180 | 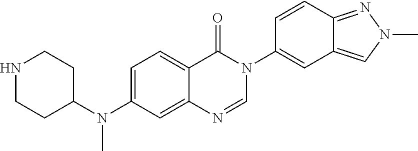 [SMILES: CN(c1ccc2c(=O)n(-c3ccc4nn(C)cc4c3)cnc2c1)C1CCNCC1] |
| 181 | 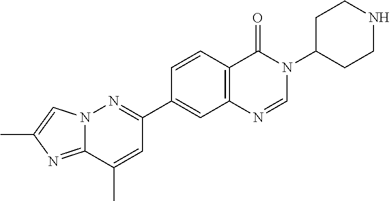 [SMILES: Cc1cn2nc(-c3ccc4c(=O)n(C5CCNCC5)cnc4c3)cc(C)c2n1] |
| 182 | 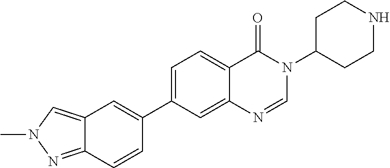 [SMILES: Cn1cc2cc(-c3ccc4c(=O)n(C5CCNCC5)cnc4c3)ccc2n1] |
| 203 | 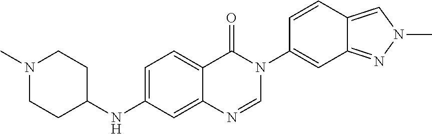 |
| 204 | 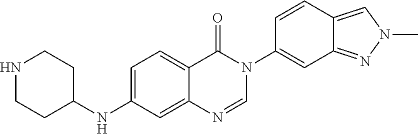 [SMILES: Cn1cc2ccc(-n3cnc4cc(NC5CCNCC5)ccc4c3=O)cc2n1] |
| 205 | 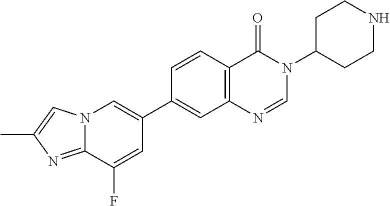 [SMILES: Cc1cn2cc(-c3ccc4c(=O)n(C5CCNCC5)cnc4c3)cc(F)c2n1] |
| 206 | 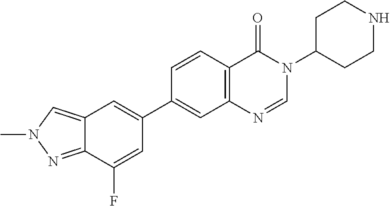 [SMILES: Cn1cc2cc(-c3ccc4c(=O)n(C5CCNCC5)cnc4c3)cc(F)c2n1] |
| 207 | 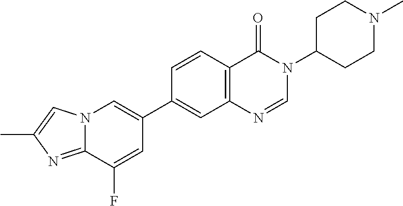 |
| 208 | 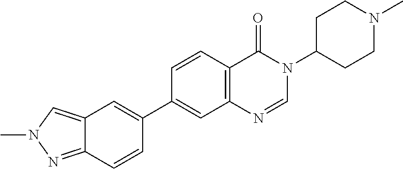 |
| 209 | 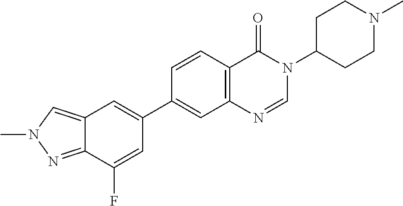 |
| 210 | 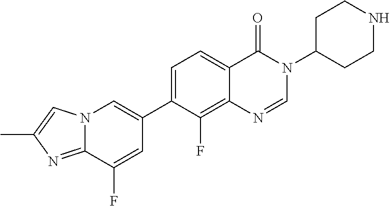 [SMILES: Cc1cn2cc(-c3ccc4c(=O)n(C5CCNCC5)cnc4c3F)cc(F)c2n1] |
| 227 | 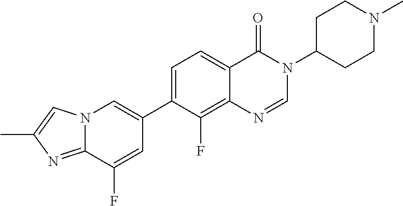 |
| 228 | 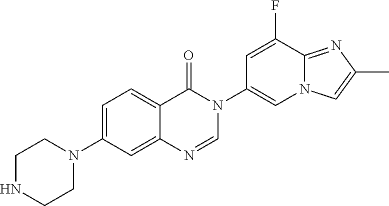 [SMILES: Cc1cn2cc(-n3cnc4cc(N5CCNCC5)ccc4c3=O)cc(F)c2n1] |
| 229 | 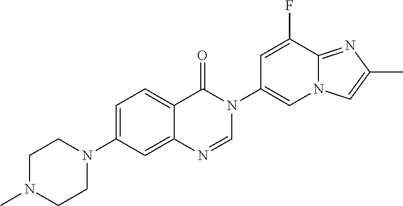 |
| 230 | 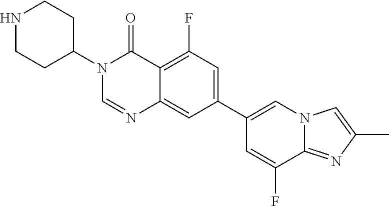 [SMILES: Cc1cn2cc(-c3cc(F)c4c(=O)n(C5CCNCC5)cnc4c3)cc(F)c2n1] |
| 231 | 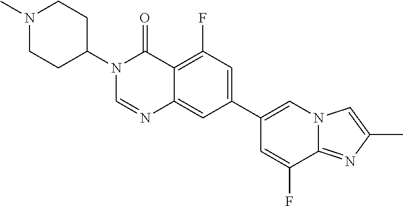 |
| 232 | 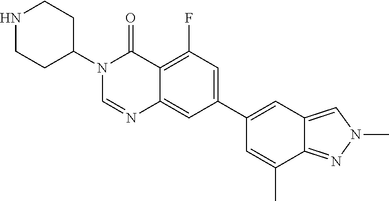 [SMILES: Cc1cc(-c2cc(F)c3c(=O)n(C4CCNCC4)cnc3c2)cc2cn(C)nc12] |
| 233 | 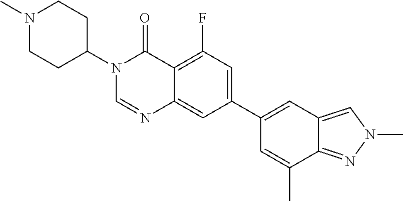 |
| 234 | 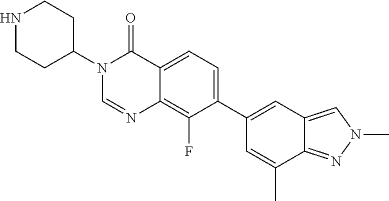 [SMILES: Cc1cc(-c2ccc3c(=O)n(C4CCNCC4)cnc3c2F)cc2cn(C)nc12] |
| 235 | 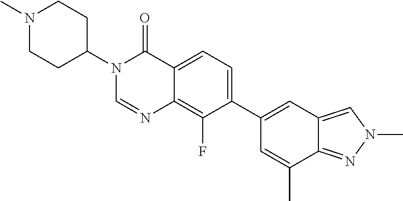 |
| 236 | 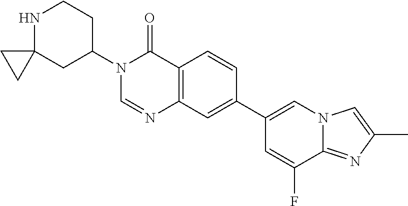 [SMILES: Cc1cn2cc(-c3ccc4c(=O)n(C5CCNC6(CC6)C5)cnc4c3)cc(F)c2n1] |
| 237 | 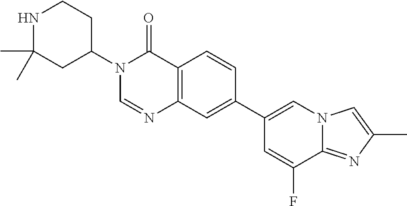 |
| 241 | 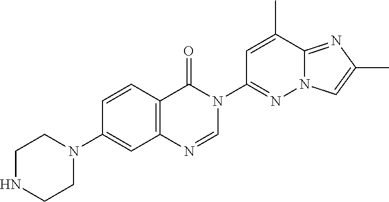 [SMILES: Cc1cn2nc(-n3cnc4cc(N5CCNCC5)ccc4c3=O)cc(C)c2n1] |
| 242 | 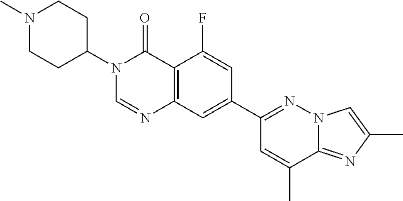 |
| 243 | 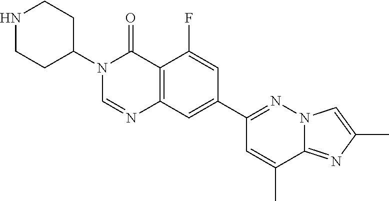 [SMILES: Cc1cn2nc(-c3cc(F)c4c(=O)n(C5CCNCC5)cnc4c3)cc(C)c2n1] |
| 244 | 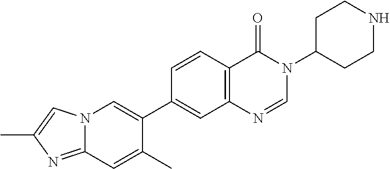 [SMILES: Cc1cn2cc(-c3ccc4c(=O)n(C5CCNCC5)cnc4c3)c(C)cc2n1] |
| 245 | 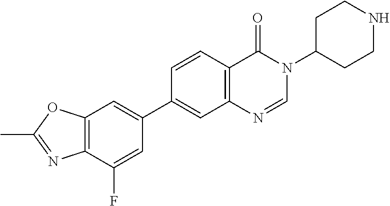 [SMILES: Cc1nc2c(F)cc(-c3ccc4c(=O)n(C5CCNCC5)cnc4c3)cc2o1] |
| 246 | 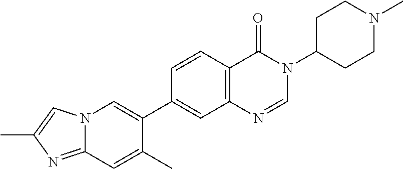 |
| 284 | 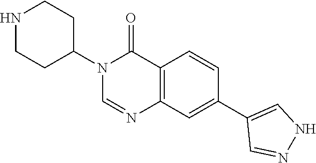 [SMILES: O=c1c2ccc(-c3cn[nH]c3)cc2ncn1C1CCNCC1] |
| 285 | 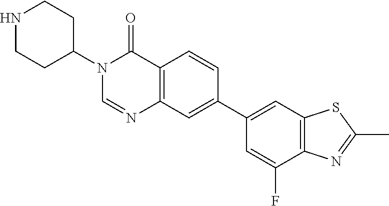 [SMILES: Cc1nc2c(F)cc(-c3ccc4c(=O)n(C5CCNCC5)cnc4c3)cc2s1] |
| 286 | 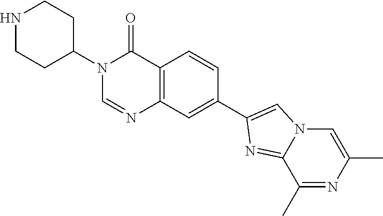 [SMILES: Cc1cn2cc(-c3ccc4c(=O)n(C5CCNCC5)cnc4c3)nc2c(C)n1] |
| 287 | 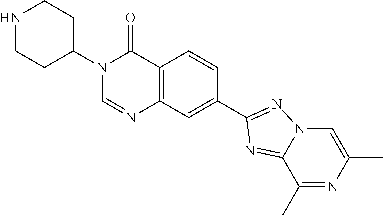 [SMILES: Cc1cn2nc(-c3ccc4c(=O)n(C5CCNCC5)cnc4c3)nc2c(C)n1] |
| 288 | 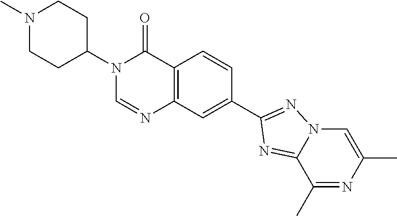 |
| 289 | 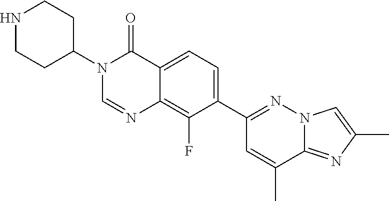 [SMILES: Cc1cn2nc(-c3ccc4c(=O)n(C5CCNCC5)cnc4c3F)cc(C)c2n1] |
| 290 | 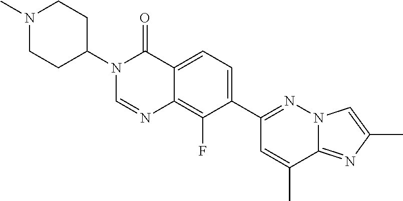 |
| 291 | 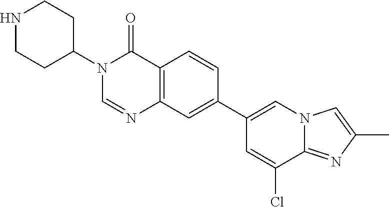 [SMILES: Cc1cn2cc(-c3ccc4c(=O)n(C5CCNCC5)cnc4c3)cc(Cl)c2n1] |
| 292 | 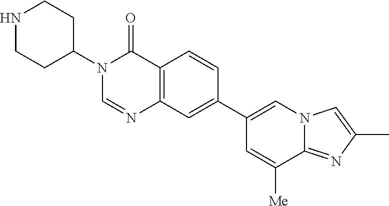 [SMILES: Cc1cn2cc(-c3ccc4c(=O)n(C5CCNCC5)cnc4c3)cc(C)c2n1] |
| 293 | 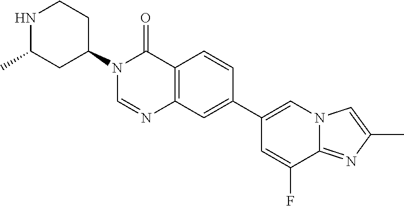 |
| 294 | 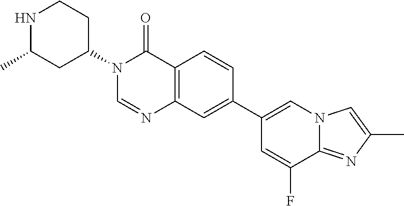 |
| 295 | 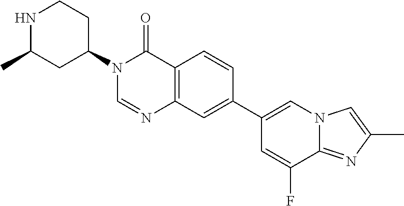 |
| 296 | 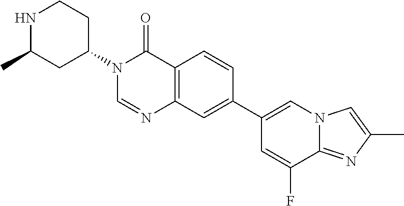 |
| 297 | 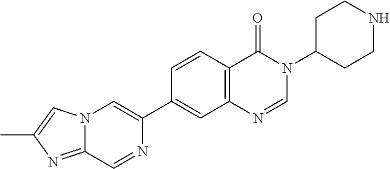 [SMILES: Cc1cn2cc(-c3ccc4c(=O)n(C5CCNCC5)cnc4c3)ncc2n1] |
| 298 | 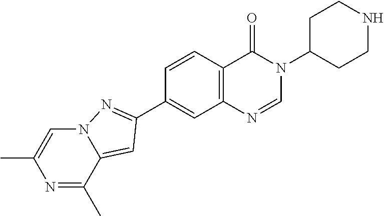 [SMILES: Cc1cn2nc(-c3ccc4c(=O)n(C5CCNCC5)cnc4c3)cc2c(C)n1] |
| 299 | 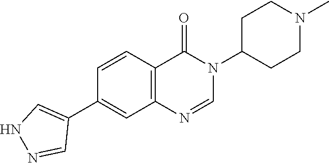 [SMILES: CN1CCC(n2cnc3cc(-c4cn[nH]c4)ccc3c2=O)CC1] |
| 300 | 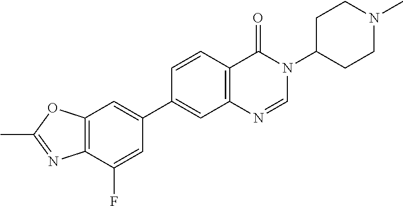 |
| 301 | 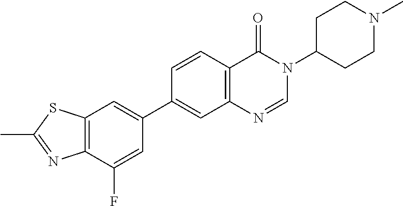 |
| 302 | 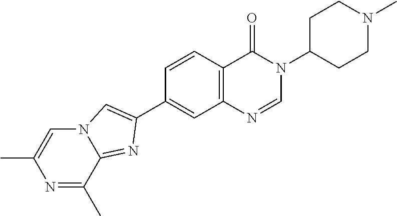 |
| 303 | 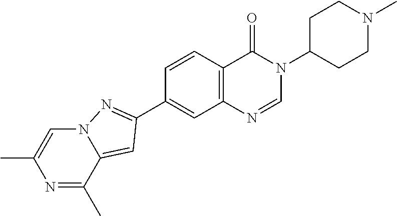 |
| 306 | 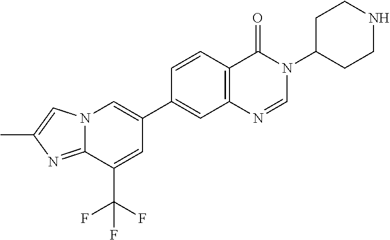 [SMILES: Cc1cn2cc(-c3ccc4c(=O)n(C5CCNCC5)cnc4c3)cc(C(F)(F)F)c2n1] |
| 307 | 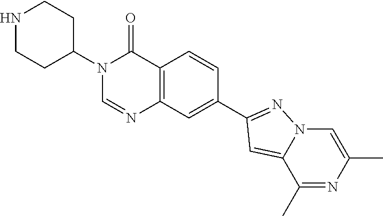 [SMILES: Cc1cn2nc(-c3ccc4c(=O)n(C5CCNCC5)cnc4c3)cc2c(C)n1] |
| 308 | 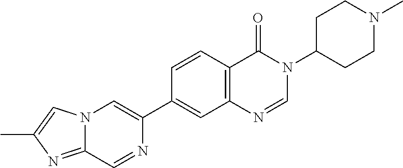 |
| 311 | 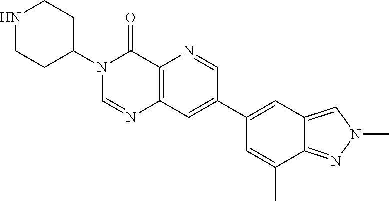 [SMILES: Cc1cc(-c2cnc3c(=O)n(C4CCNCC4)cnc3c2)cc2cn(C)nc12] |
| 313 | 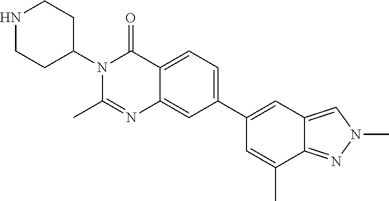 [SMILES: Cc1cc(-c2ccc3c(=O)n(C4CCNCC4)c(C)nc3c2)cc2cn(C)nc12] |
| 314 | 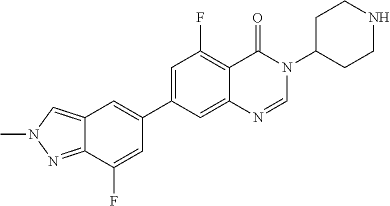 [SMILES: Cn1cc2cc(-c3cc(F)c4c(=O)n(C5CCNCC5)cnc4c3)cc(F)c2n1] |
| 315 | 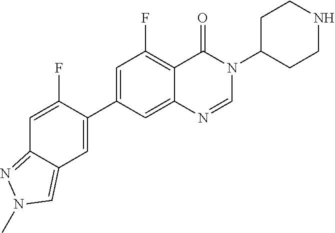 [SMILES: Cn1cc2cc(-c3cc(F)c4c(=O)n(C5CCNCC5)cnc4c3)c(F)cc2n1] |
| 316 | 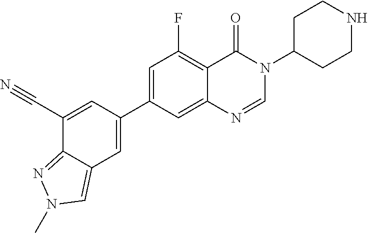 [SMILES: Cn1cc2cc(-c3cc(F)c4c(=O)n(C5CCNCC5)cnc4c3)cc(C#N)c2n1] |
| 317 | 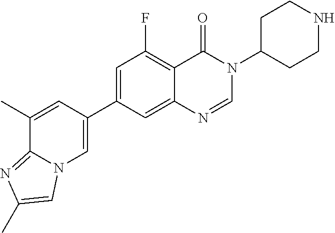 [SMILES: Cc1cn2cc(-c3cc(F)c4c(=O)n(C5CCNCC5)cnc4c3)cc(C)c2n1] |
| 318 | 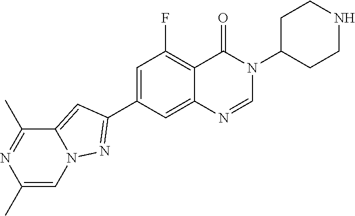 [SMILES: Cc1cn2nc(-c3cc(F)c4c(=O)n(C5CCNCC5)cnc4c3)cc2c(C)n1] |
| 319 | 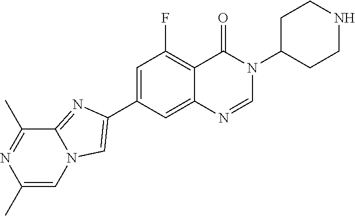 [SMILES: Cc1cn2cc(-c3cc(F)c4c(=O)n(C5CCNCC5)cnc4c3)nc2c(C)n1] |
| 320 | 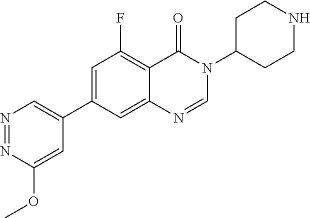 [SMILES: COc1cc(-c2cc(F)c3c(=O)n(C4CCNCC4)cnc3c2)cnn1] |
| 321 | 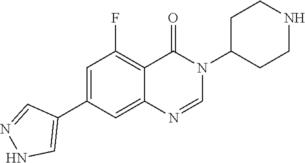 [SMILES: O=c1c2c(F)cc(-c3cn[nH]c3)cc2ncn1C1CCNCC1] |
| 323 | 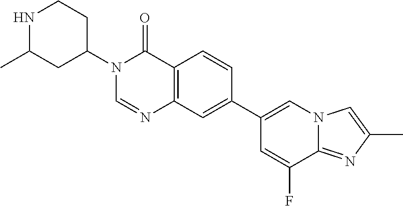 |

In some embodiments, for Formula (III), A is monocyclic heterocyclyl (e.g., N-methyl piperazyl); B is bicyclic heterocyclyl (e.g., 2-methyl-2H-indazolyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), (III-b), and (III-c) is Compound 152, or a pharmaceutically acceptable salt, solvate, hydrate, tautomer, or stereoisomer thereof.

In some embodiments, for Formula (III), A is monocyclic heterocyclyl (e.g., N-methyl piperazyl); B is bicyclic heterocyclyl (e.g., 2-methyl-2H-indazolyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), (III-b), and (III-c) is Compound 153, or a pharmaceutically acceptable salt, solvate, hydrate, tautomer, or stereoisomer thereof.

In some embodiments, for Formula (III), A is bicyclic heterocyclyl (e.g., 2,7-dimethyl-2H-indazolyl); B is monocyclic heterocyclyl (e.g., N-methyl piperidinyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), (III-b), and (III-c) is Compound 156, or a pharmaceutically acceptable salt, solvate, hydrate, tautomer, or stereoisomer thereof.

In some embodiments, for Formula (III), A is bicyclic heterocyclyl (e.g., 2,7-dimethyl-2H-indazolyl); B is monocyclic heterocyclyl (e.g., 2,2,6,6-tetramethylpiperidinyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), (III-b), and (III-c) is Compound 157, or a pharmaceutically acceptable salt, solvate, hydrate, tautomer, or stereoisomer thereof.

In some embodiments, for Formula (III), A is monocyclic heterocyclyl (e.g., 1,2,3,6-tetrahydropyridinyl); B is bicyclic heterocyclyl (e.g., 2-methyl-2H-indazolyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), (III-b), and (III-c) is Compound 158, or a pharmaceutically acceptable salt, solvate, hydrate, tautomer, or stereoisomer thereof.

In some embodiments, for Formula (III), A is monocyclic heterocyclyl (e.g., N-methyl 1,2,3,6-tetrahydropyridinyl); B is bicyclic heterocyclyl (e.g., 2-methyl-2H-indazolyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), (III-b), and (III-c) is Compound 159, or a pharmaceutically acceptable salt, solvate, hydrate, tautomer, or stereoisomer thereof.

In some embodiments, for Formula (III), A is monocyclic heterocyclyl (e.g., 8-azabicyclo[3.2.1]oct-2-enyl); B is bicyclic heterocyclyl (e.g., 2-methyl-2H-indazolyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), (III-b), and (III-c) is Compound 160, or a pharmaceutically acceptable salt, solvate, hydrate, tautomer, or stereoisomer thereof.

In some embodiments, for Formula (III), A is monocyclic heterocyclyl (e.g., N-methyl 8-azabicyclo[3.2.1]oct-2-enyl); B is bicyclic heterocyclyl (e.g., 2-methyl-2H-indazolyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), (III-b), and (III-c) is Compound 161, or a pharmaceutically acceptable salt, solvate, hydrate, tautomer, or stereoisomer thereof.

In some embodiments, for Formula (III), A is monocyclic heterocyclyl (e.g., piperidinyl); B is bicyclic heterocyclyl (e.g., 2-methyl-2H-indazolyl); L¹ is —N(R⁸)— (e.g., —NH—); L² are absent; X and Z are each independently C(R³) (e.g., CH); Y is N; R² is absent; and R^{7a} and R^{7b} are each independently hydrogen. In some embodiments, the compound of Formulas (III), (III-a), (III-b), and (III-c) is Compound 162, or a pharmaceutically acceptable salt, solvate, hydrate, tautomer, or stereoisomer thereof.

In some embodiments, for Formula (III), A is monocyclic heterocyclyl (e.g., N-methyl piperidinyl); B is bicyclic heterocyclyl (e.g., 2-methyl-2H-indazolyl); L¹ is —N(R⁸)— (e.g., —NH—); L² are absent; X and Z are each independently C(R³) (e.g., CH); Y is N; R² is absent; and R^{7a} and R^{7b} are each independently hydrogen. In some embodiments, the compound of Formulas (III), (III-a), (III-b), and (III-c) is Compound 163, or a pharmaceutically acceptable salt, solvate, hydrate, tautomer, or stereoisomer thereof.

In some embodiments, for Formula (III), A is monocyclic heterocyclyl (e.g., piperidinyl); B is bicyclic heterocyclyl (e.g., 2-methyl-2H-indazolyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), (III-b), and (III-c) is Compound 172, or a pharmaceutically acceptable salt, solvate, hydrate, tautomer, or stereoisomer thereof.

In some embodiments, for Formula (III), A is bicyclic heterocyclyl (e.g., 2,7-dimethyl-2H-indazolyl); B is monocyclic heterocyclyl (e.g., N-methyl piperidinyl); L¹ and L² are absent; X is C(R³) (e.g., CH); Z and Y are each independently N; R² is absent; and R^{7a} and R^{7b} are each independently hydrogen. In some embodiments, the compound of Formulas (III), (III-a), and (III-b) is Compound 173, or a pharmaceutically acceptable salt, solvate, hydrate, tautomer, or stereoisomer thereof.

In some embodiments, for Formula (III), A is bicyclic heterocyclyl (e.g., 2,7-dimethyl-2H-indazolyl); B is monocyclic heterocyclyl (e.g., piperidinyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), (III-b), and (III-c) is Compound 174, or a pharmaceutically acceptable salt, solvate, hydrate, tautomer, or stereoisomer thereof.

In some embodiments, for Formula (III), A is bicyclic heterocyclyl (e.g., 2,7-dimethyl-2H-indazolyl); B is monocyclic heterocyclyl (e.g., N-methyl piperazyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), (III-b), and (III-c) is Compound 175, or a pharmaceutically acceptable salt, solvate, hydrate, tautomer, or stereoisomer thereof.

In some embodiments, for Formula (III), A is bicyclic heterocyclyl (e.g., 2,8-dimethylimidazo[1,2-b]pyridazyl); B is monocyclic heterocyclyl (e.g., N-methyl piperidinyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), (III-b), and (III-c) is Compound 176, or a pharmaceutically acceptable salt, solvate, hydrate, tautomer, or stereoisomer thereof.

In some embodiments, for Formula (III), A is monocyclic heterocyclyl (e.g., N-methyl piperidinyl); B is bicyclic heterocyclyl (e.g., 2-methyl-2H-indazolyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), (III-b), and (III-c) is Compound 177, or a pharmaceutically acceptable salt, solvate, hydrate, tautomer, or stereoisomer thereof.

In some embodiments, for Formula (III), A is monocyclic heterocyclyl (e.g., N-methyl piperazyl); B is bicyclic heterocyclyl (e.g., 2-methyl-2H-indazolyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), (III-b), and (III-c) is Compound 178, or a pharmaceutically acceptable salt, solvate, hydrate, tautomer, or stereoisomer thereof.

In some embodiments, for Formula (III), A is bicyclic heterocyclyl (e.g., 2,7-dimethyl-2H-indazolyl); B is monocyclic heterocyclyl (e.g., 2,2,6,6-tetramethylpiperidinyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), (III-b), and (III-c) is Compound 179, or a pharmaceutically acceptable salt, solvate, hydrate, tautomer, or stereoisomer thereof.

In some embodiments, for Formula (III), A is monocyclic heterocyclyl (e.g., piperidinyl); B is bicyclic heterocyclyl (e.g., 2-methyl-2H-indazolyl); L¹ is —N(R⁸)— (e.g., —N(CH₃)—); L² is absent; X and Z are each independently C(R³) (e.g., CH); Y is N; R² is absent; and R^{7a} and R^{7b} are each independently hydrogen. In some embodiments, the compound of Formulas (III), (III-a), (III-b), and (III-c) is Compound 180, or a pharmaceutically acceptable salt, solvate, hydrate, tautomer, or stereoisomer thereof.

In some embodiments, for Formula (III), A is bicyclic heterocyclyl (e.g., 2,8-dimethylimidazo[1,2-b]pyridazinyl); B is monocyclic heterocyclyl (e.g., piperidinyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), (III-b), and (III-c) is Compound 181, or a pharmaceutically acceptable salt, solvate, hydrate, tautomer, or stereoisomer thereof.

In some embodiments, for Formula (III), A is bicyclic heterocyclyl (e.g., 2-methyl-2H-indazolyl); B is monocyclic heterocyclyl (e.g., piperidinyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), (III-b), and (III-c) is Compound 182, or a pharmaceutically acceptable salt, solvate, hydrate, tautomer, or stereoisomer thereof.

In some embodiments, for Formula (III), A is monocyclic heterocyclyl (e.g., N-methyl piperidinyl); B is bicyclic heterocyclyl (e.g., 2-methyl-2H-indazolyl); L¹ is —N(R⁸)— (e.g., —NH—); L² is absent; X and Z are each independently C(R³) (e.g., CH); Y is N; R² is absent; and R^{7a} and R^{7b} are each independently hydrogen. In some embodiments, the compound of Formulas (III), (III-a), (III-b), and (III-c) is Compound 203, or a pharmaceutically acceptable salt, solvate, hydrate, tautomer, or stereoisomer thereof.

In some embodiments, for Formula (III), A is monocyclic heterocyclyl (e.g., piperidinyl); B is bicyclic heterocyclyl (e.g., 2-methyl-2H-indazolyl); L¹ is —N(R⁸)— (e.g., —NH—); L² is absent; X and Z are each independently C(R³) (e.g., CH); Y is N; R² is absent; and R^{7a} and R^{7b} are each independently hydrogen. In some embodiments, the compound of Formulas (III), (III-a), (III-b), and (III-c) is Compound 204, or a pharmaceutically acceptable salt, solvate, hydrate, tautomer, or stereoisomer thereof.

In some embodiments, for Formula (III), A is bicyclic heterocyclyl (e.g., 8-fluoro-2-methylimidazo[1,2-a]pyridinyl); B is monocyclic heterocyclyl (e.g., piperidinyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), (III-b), and (III-c) is Compound 205, or a pharmaceutically acceptable salt, solvate, hydrate, tautomer, or stereoisomer thereof.

In some embodiments, for Formula (III), A is bicyclic heterocyclyl (e.g., 7-fluoro-2-methyl-2H-indazolyl); B is monocyclic heterocyclyl (e.g., piperidinyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), (III-b), and (III-c) is Compound 206, or a pharmaceutically acceptable salt, solvate, hydrate, tautomer, or stereoisomer thereof.

In some embodiments, for Formula (III), A is bicyclic heterocyclyl (e.g., 8-fluoro-2-methylimidazo[1,2-a]pyridinyl); B is monocyclic heterocyclyl (e.g., N-methyl piperidinyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), (III-b), and (III-c) is Compound 207, or a pharmaceutically acceptable salt, solvate, hydrate, tautomer, or stereoisomer thereof.

In some embodiments, for Formula (III), A is bicyclic heterocyclyl (e.g., 2-methyl-2H-indazolyl); B is monocyclic heterocyclyl (e.g., N-methyl piperidinyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), (III-b), and (III-c) is Compound 208, or a pharmaceutically acceptable salt, solvate, hydrate, tautomer, or stereoisomer thereof.

In some embodiments, for Formula (III), A is bicyclic heterocyclyl (e.g., 7-fluoro-2-methyl-2H-indazolyl); B is monocyclic heterocyclyl (e.g., N-methyl piperidinyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), (III-b), and (III-c) is Compound 209, or a pharmaceutically acceptable salt, solvate, hydrate, tautomer, or stereoisomer thereof.

In some embodiments, for Formula (III), A is bicyclic heterocyclyl (e.g., 8-fluoro-2-methylimidazo[1,2-a]pyridinyl); B is monocyclic heterocyclyl (e.g., piperidinyl); L¹ and L² are absent; X is C(R³) (e.g., CH); Z is C(R³) (e.g., CF); Y is N; R² is absent; and R^{7a} and R^{7b} are each independently hydrogen. In some embodiments, the compound of Formulas (III), (III-a), and (III-b) is Compound 210, or a pharmaceutically acceptable salt, solvate, hydrate, tautomer, or stereoisomer thereof.

In some embodiments, for Formula (III), A is bicyclic heterocyclyl (e.g., 8-fluoro-2-methylimidazo[1,2-a]pyridinyl); B is monocyclic heterocyclyl (e.g., N-methyl piperidinyl); L¹ and L² are absent; X is C(R³) (e.g., CH); Z is C(R³) (e.g., CF); Y is N; R² is absent; and R^{7a} and R^{7b} are each independently hydrogen. In some embodiments, the compound of Formulas (III), (III-a), and (III-b) is Compound 227, or a pharmaceutically acceptable salt, solvate, hydrate, tautomer, or stereoisomer thereof.

In some embodiments, for Formula (III), A is monocyclic heterocyclyl (e.g., piperazyl); B is bicyclic heterocyclyl (e.g., 8-fluoro-2-methylimidazo[1,2-a]pyridinyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), (III-b), and (III-c) is Compound 228, or a pharmaceutically acceptable salt, solvate, hydrate, tautomer, or stereoisomer thereof.

In some embodiments, for Formula (III), A is monocyclic heterocyclyl (e.g., N-methyl piperazyl); B is bicyclic heterocyclyl (e.g., 8-fluoro-2-methylimidazo[1,2-a]pyridinyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), (III-b), and (III-c) is Compound 229, or a pharmaceutically acceptable salt, solvate, hydrate, tautomer, or stereoisomer thereof.

In some embodiments, for Formula (III), A is bicyclic heterocyclyl (e.g., 8-fluoro-2-methylimidazo[1,2-a]pyridinyl); B is monocyclic heterocyclyl (e.g., piperidinyl); L¹ and L² are absent; X is C(R³) (e.g., CF); Z is C(R³) (e.g., CH); Y is N; R² is absent; and R^{7a} and R^{7b} are each independently hydrogen. In some embodiments, the compound of Formulas (III), (III-a), and (III-b) is Compound 230, or a pharmaceutically acceptable salt, solvate, hydrate, tautomer, or stereoisomer thereof.

In some embodiments, for Formula (III), A is bicyclic heterocyclyl (e.g., 8-fluoro-2-methylimidazo[1,2-a]pyridinyl); B is monocyclic heterocyclyl (e.g., N-methyl piperidinyl); L¹ and L² are absent; X is C(R³) (e.g., CF); Z is C(R³) (e.g., CH); Y is N; R² is absent; and R^{7a} and R^{7b} are each independently hydrogen. In some embodiments, the compound of Formulas (III), (III-a), and (III-b) is Compound 231, or a pharmaceutically acceptable salt, solvate, hydrate, tautomer, or stereoisomer thereof.

In some embodiments, for Formula (III), A is bicyclic heterocyclyl (e.g., 2,7-dimethyl-2H-indazolyl); B is monocyclic heterocyclyl (e.g., piperidinyl); L¹ and L² are absent; X is C(R³) (e.g., CF); Z is C(R³) (e.g., CH); Y is N; R² is absent; and R^{7a} and R^{7b} are each independently hydrogen. In some embodiments, the compound of Formulas (III), (III-a), and (III-b) is Compound 232, or a pharmaceutically acceptable salt, solvate, hydrate, tautomer, or stereoisomer thereof.

In some embodiments, for Formula (III), A is bicyclic heterocyclyl (e.g., 2,7-dimethyl-2H-indazolyl); B is monocyclic heterocyclyl (e.g., N-methyl piperidinyl); L¹ and L² are absent; X is C(R³) (e.g., CF); Z is C(R³) (e.g., CH); Y is N; R² is absent; and R^{7a} and R^{7b} are each independently hydrogen. In some embodiments, the compound of Formulas (III), (III-a), and (III-b) is Compound 233, or a pharmaceutically acceptable salt, solvate, hydrate, tautomer, or stereoisomer thereof.

In some embodiments, for Formula (III), A is bicyclic heterocyclyl (e.g., 2,7-dimethyl-2H-indazolyl); B is monocyclic heterocyclyl (e.g., piperidinyl); L¹ and L² are absent; X is C(R³) (e.g., Ch); Z is C(R³) (e.g., CF); Y is N; R² is absent; and R^{7a} and R^{7b} are each independently hydrogen. In some embodiments, the compound of Formulas (III), (III-a), and (III-b) is Compound 234, or a pharmaceutically acceptable salt, solvate, hydrate, tautomer, or stereoisomer thereof.

In some embodiments, for Formula (III), A is bicyclic heterocyclyl (e.g., 2,7-dimethyl-2H-indazolyl); B is monocyclic heterocyclyl (e.g., N-methyl piperidinyl); L¹ and L² are absent; X is C(R³) (e.g., Ch); Z is C(R³) (e.g., CF); Y is N; R² is absent; and R^{7a} and R^{7b} are each independently hydrogen. In some embodiments, the compound of Formulas (III), (III-a), and (III-b) is Compound 235, or a pharmaceutically acceptable salt, solvate, hydrate, tautomer, or stereoisomer thereof.

In some embodiments, for Formula (III), A is bicyclic heterocyclyl (e.g., 8-fluoro-2-methylimidazo[1,2-a]pyridinyl); B is monocyclic heterocyclyl (e.g., 4-azaspiro[2.5]octanyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), (III-b), and (III-c) is Compound 236, or a pharmaceutically acceptable salt, solvate, hydrate, tautomer, or stereoisomer thereof.

In some embodiments, for Formula (III), A is bicyclic heterocyclyl (e.g., 8-fluoro-2-methylimidazo[1,2-a]pyridinyl); B is monocyclic heterocyclyl (e.g., 2,2-dimethylpiperidinyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), (III-b), and (III-c) is Compound 237, or a pharmaceutically acceptable salt, solvate, hydrate, tautomer, or stereoisomer thereof.

In some embodiments, for Formula (III), A is monocyclic heterocyclyl (e.g., piperazyl); B is bicyclic heterocyclyl (e.g., 2,8-dimethylimidazo[1,2-b]pyridazyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), (III-b), and (III-c) is Compound 241, or a pharmaceutically acceptable salt, solvate, hydrate, tautomer, or stereoisomer thereof. In some embodiments, for Formula (III), A is bicyclic heterocyclyl (e.g., 2,8-dimethylimidazo[1,2-b]pyridazyl); B is monocyclic heterocyclyl (e.g., N-methyl piperidinyl); L¹ and L² are absent; X is C(R³) (e.g., CF); Z is C(R³) (e.g., CH); Y is N; R² is absent; and R^{7a} and R^{7b} are each independently hydrogen. In some embodiments, the compound of Formulas (III), (III-a), and (III-b) is Compound 242, or a pharmaceutically acceptable salt, solvate, hydrate, tautomer, or stereoisomer thereof.

In some embodiments, for Formula (III), A is bicyclic heterocyclyl (e.g., 2,8-dimethylimidazo[1,2-b]pyridazyl); B is monocyclic heterocyclyl (e.g., piperidinyl); L¹ and L² are absent; X is C(R³) (e.g., CF); Z is C(R³) (e.g., CH); Y is N; R² is absent; and R^{7a} and R^{7b} are each independently hydrogen. In some embodiments, the compound of Formulas (III), (III-a), and (III-b) is Compound 243, or a pharmaceutically acceptable salt, solvate, hydrate, tautomer, or stereoisomer thereof.

In some embodiments, for Formula (III), A is bicyclic heterocyclyl (e.g., 2,7-dimethylimidazo[1,2-a]pyridinyl); B is monocyclic heterocyclyl (e.g., piperidinyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), (III-b), and (III-c) is Compound 244, or a pharmaceutically acceptable salt, solvate, hydrate, tautomer, or stereoisomer thereof.

In some embodiments, for Formula (III), A is bicyclic heterocyclyl (e.g., 4-fluoro-2-methylbenzo[d]oxazolyl); B is monocyclic heterocyclyl (e.g., piperidinyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), (III-b), and (III-c) is Compound 245, or a pharmaceutically acceptable salt, solvate, hydrate, tautomer, or stereoisomer thereof.

In some embodiments, for Formula (III), A is bicyclic heterocyclyl (e.g., 2,7-dimethylimidazo[1,2-a]pyridinyl); B is monocyclic heterocyclyl (e.g., N-methyl piperidinyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), (III-b), and (III-c) is Compound 246, or a pharmaceutically acceptable salt, solvate, hydrate, tautomer, or stereoisomer thereof.

In some embodiments, for Formula (III), A is monocyclic heterocyclyl (e.g., pyrazolyl); B is monocyclic heterocyclyl (e.g., piperidinyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), (III-b), and (III-c) is Compound 284, or a pharmaceutically acceptable salt, solvate, hydrate, tautomer, or stereoisomer thereof.

In some embodiments, for Formula (III), A is bicyclic heterocyclyl (e.g., 4-fluoro-2-methylbenzo[d]thiazolyl); B is monocyclic heterocyclyl (e.g., piperidinyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), (III-b), and (III-c) is Compound 285, or a pharmaceutically acceptable salt, solvate, hydrate, tautomer, or stereoisomer thereof.

In some embodiments, for Formula (III), A is bicyclic heterocyclyl (e.g., 6,8-dimethylimidazo[1,2-a]pyrazyl); B is monocyclic heterocyclyl (e.g., piperidinyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), (III-b), and (III-c) is Compound 286, or a pharmaceutically acceptable salt, solvate, hydrate, tautomer, or stereoisomer thereof.

In some embodiments, for Formula (III), A is bicyclic heterocyclyl (e.g., 6,8-dimethyl-[1,2,4]triazolo[1,5-a]pyrazinyl); B is monocyclic heterocyclyl (e.g., piperidinyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), (III-b), and (III-c) is Compound 287, or a pharmaceutically acceptable salt, solvate, hydrate, tautomer, or stereoisomer thereof.

In some embodiments, for Formula (III), A is bicyclic heterocyclyl (e.g., 6,8-dimethyl-[1,2,4]triazolo[1,5-a]pyrazinyl); B is monocyclic heterocyclyl (e.g., N-methyl piperidinyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), (III-b), and (III-c) is Compound 288, or a pharmaceutically acceptable salt, solvate, hydrate, tautomer, or stereoisomer thereof.

In some embodiments, for Formula (III), A is bicyclic heterocyclyl (e.g., 2,8-dimethylimidazo[1,2-b]pyridazyl); B is monocyclic heterocyclyl (e.g., piperidinyl); L¹ and L² are absent; X is C(R³) (e.g., CH); Z is C(R³) (e.g., CF); Y is N; R² is absent; and R^{7a} and R^{7b} are each independently hydrogen. In some embodiments, the compound of Formulas (III), (III-a), and (III-b) is Compound 289, or a pharmaceutically acceptable salt, solvate, hydrate, tautomer, or stereoisomer thereof.

In some embodiments, for Formula (III), A is bicyclic heterocyclyl (e.g., 2,8-dimethylimidazo[1,2-b]pyridazyl); B is monocyclic heterocyclyl (e.g., N-methyl piperidinyl); L¹ and L² are absent; X is C(R³) (e.g., CH); Z is C(R³) (e.g., CF); Y is N; R² is absent; and R^{7a} and R^{7b} are each independently hydrogen. In some embodiments, the compound of Formulas (III), (III-a), and (III-b) is Compound 290, or a pharmaceutically acceptable salt, solvate, hydrate, tautomer, or stereoisomer thereof.

In some embodiments, for Formula (III), A is bicyclic heterocyclyl (e.g., 8-chloro-2-methylimidazo[1,2-a]pyridinyl); B is monocyclic heterocyclyl (e.g., piperidinyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), (III-b), and (III-c) is Compound 291, or a pharmaceutically acceptable salt, solvate, hydrate, tautomer, or stereoisomer thereof.

In some embodiments, for Formula (III), A is bicyclic heterocyclyl (e.g., 2,8-dimethylimidazo[1,2-a]pyridinyl); B is monocyclic heterocyclyl (e.g., piperidinyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), (III-b), and (III-c) is Compound 292, or a pharmaceutically acceptable salt, solvate, hydrate, tautomer, or stereoisomer thereof.

In some embodiments, for Formula (III), A is bicyclic heterocyclyl (e.g., 8-fluoro-2-methylimidazo[1,2-a]pyridinyl); B is monocyclic heterocyclyl (e.g., 2-methyl piperidinyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), (III-b), and (III-c) is Compound 293, 294, 295, 296, or 323, or a pharmaceutically acceptable salt, solvate, hydrate, tautomer, or stereoisomer thereof.

In some embodiments, for Formula (III), A is bicyclic heterocyclyl (e.g., 2-methylimidazo[1,2-a]pyrazyl); B is monocyclic heterocyclyl (e.g., piperidinyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), (III-b), and (III-c) is Compound 297, or a pharmaceutically acceptable salt, solvate, hydrate, tautomer, or stereoisomer thereof.

In some embodiments, for Formula (III), A is bicyclic heterocyclyl (e.g., 4,6-dimethylpyrazolo[1,5-a]pyrazyl); B is monocyclic heterocyclyl (e.g., piperidinyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), (III-b), and (III-c) is Compound 298, or a pharmaceutically acceptable salt, solvate, hydrate, tautomer, or stereoisomer thereof.

In some embodiments, for Formula (III), A is monocyclic heterocyclyl (e.g., pyrazolyl); B is monocyclic heterocyclyl (e.g., N-methyl piperidinyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), (III-b), and (III-c) is Compound 299, or a pharmaceutically acceptable salt, solvate, hydrate, tautomer, or stereoisomer thereof.

In some embodiments, for Formula (III), A is bicyclic heterocyclyl (e.g., 4-fluoro methylbenzo[d]oxazolyl); B is monocyclic heterocyclyl (e.g., N-methyl piperidinyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), (III-b), and (III-c) is Compound 300, or a pharmaceutically acceptable salt, solvate, hydrate, tautomer, or stereoisomer thereof.

In some embodiments, for Formula (III), A is bicyclic heterocyclyl (e.g., 4-fluoro-2-methylbenzo[d]thiazolyl); B is monocyclic heterocyclyl (e.g., N-methyl piperidinyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), (III-b), and (III-c) is Compound 301, or a pharmaceutically acceptable salt, solvate, hydrate, tautomer, or stereoisomer thereof.

In some embodiments, for Formula (III), A is bicyclic heterocyclyl (e.g., 6,8-dimethylimidazo[1,2-a]pyrazyl); B is monocyclic heterocyclyl (e.g., N-methyl piperidinyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), (III-b), and (III-c) is Compound 302, or a pharmaceutically acceptable salt, solvate, hydrate, tautomer, or stereoisomer thereof.

In some embodiments, for Formula (III), A is bicyclic heterocyclyl (e.g., 4,6-dimethylpyrazolo[1,5-a]pyrazyl); B is monocyclic heterocyclyl (e.g., N-methyl piperidinyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), (III-b), and (III-c) is Compound 303, or a pharmaceutically acceptable salt, solvate, hydrate, tautomer, or stereoisomer thereof.

In some embodiments, for Formula (III), A is bicyclic heterocyclyl (e.g., 4,6-dimethylpyrazolo[1,5-a]pyrazyl); B is monocyclic heterocyclyl (e.g., piperidinyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), (III-b), and (III-c) is Compound 307, or a pharmaceutically acceptable salt, solvate, hydrate, tautomer, or stereoisomer thereof.

In some embodiments, for Formula (III), A is bicyclic heterocyclyl (e.g., 2-methylimidazo[1,2-a]pyrazyl); B is monocyclic heterocyclyl (e.g., N-methyl piperidinyl); L¹ and L² are absent; X and Z are each independently C(R³) (e.g., CH); Y is N; R² is absent; and R^{7a} and R^{7b} are each independently hydrogen. In some embodiments, the compound of Formulas (III), (III-a), (III-b), and (III-c) is Compound 308, or a pharmaceutically acceptable salt, solvate, hydrate, tautomer, or stereoisomer thereof.

In some embodiments, for Formula (III), A is a bicyclic heteroaryl not containing oxygen. In some embodiments, A is a bicyclic heteroaryl substituted by one or more R¹, wherein R¹ is not halo. In some embodiments, A is not

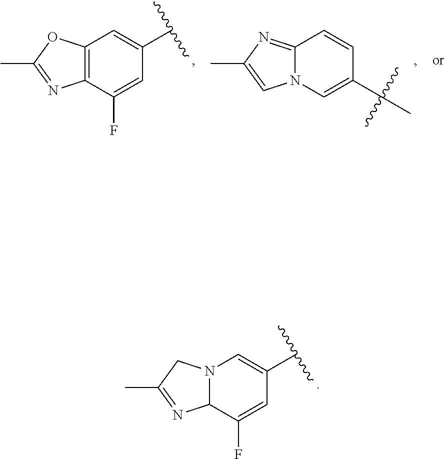 [SMILES: *c1cc(F)c2nc(C)oc2c1 ; *c1ccc2nc(C)cn2c1 ; *C1=CN2CC(C)=NC2C(F)=C1]

In some embodiments, B is a nitrogen-containing heterocyclyl optionally substituted with one or more R¹, wherein R¹ is not cycloalkyl (e.g., cyclopropyl). In some embodiments, B is unsubstituted piperidinyl (e.g., 0 R¹). In some embodiments, B is not

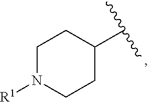

wherein R¹ is C₁-C₆ alkyl (e.g., methyl) or cycloalkyl (e.g., cyclopropyl). In some embodiments, B is

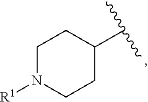

wherein R¹ is hydrogen. In some embodiments, B is not

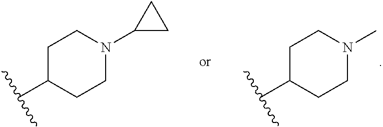

In some embodiments, B is not

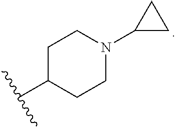 [SMILES: *C1CCN(C2CC2)CC1]

In some embodiments, X is C(R³), wherein R³ is halo. In some embodiments, X is CF.

In some embodiments, the compound of Formula (III) is not a compound disclosed in WO 2020/004594. In some embodiments, the compound of Formula (III) is not a compound selected from

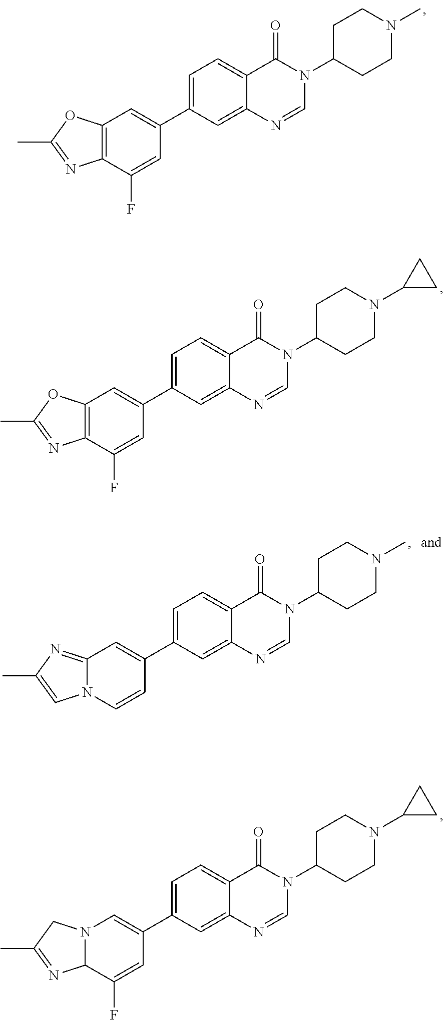

or a pharmaceutically acceptable salt thereof.

In some embodiments, the present disclosure features a compound of Formula (V-a):

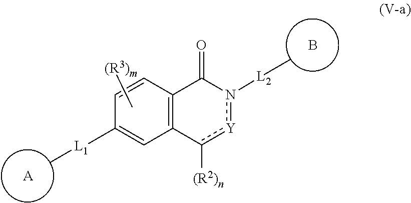

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; each of L¹ and L² is independently absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁷; Y is N, C(R^{6a}), or C(R^{6a})(R^{6b}), wherein the dashed lines in the ring comprising Y may be single or double bonds as valency permits; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁵; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵; each R² is independently hydrogen or C₁-C₆-alkyl; R³ is C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —C(O)R^{D}, or —C(O)OR^{D}; R⁴ is hydrogen, C₁-C₆-alkyl, or C₁-C₆-haloalkyl; each R⁵ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, NR^{B}R^{C}, —NR^{B}C(O)R^{D}, —NO₂, —C(O)NR^{B}R^{C}, —C(O)R^{D}, C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁷; R^{6a} and R^{6b} is independently hydrogen, C₁-C₆-alkyl, C₁-C₆-heteroalkyl, C₁-C₆-haloalkyl, or halo; each R⁷ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each R^{B} and R^{C} is independently hydrogen, C₁-C₆ alkyl, C₁-C₆ heteroalkyl, cycloalkyl, heterocyclyl, —OR^{A}; or R^{B} and R^{C} together with the atom to which they are attached form a 3-7-membered heterocyclyl ring optionally substituted with one or more R⁹; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁹ is independently C₁-C₆-alkyl or halo; n is 0, 1, or 2; m is 0, 1, 2, or 3; and x is 0, 1, or 2.

In some embodiments, A is heterocyclyl optionally substituted with one or more R¹. In some embodiments, A is monocyclic nitrogen-containing heterocyclyl. In some embodiments, A is optionally substituted piperidinyl. In some embodiments, A is selected from

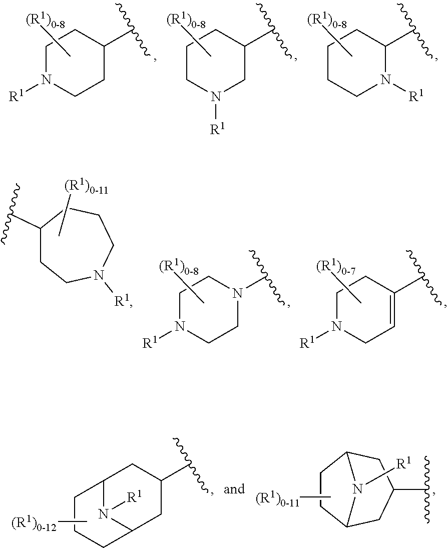

wherein R¹ is as defined herein.

In some embodiments, A is selected from,

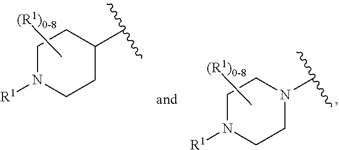

wherein R¹ is as defined herein.

In some embodiments, A is selected from

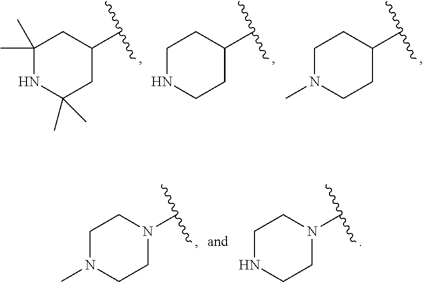

In some embodiments, A is heteroaryl. In some embodiments, A is a nitrogen-containing heteroaryl. In some embodiments, A is a bicyclic nitrogen-containing heteroaryl.

In some embodiments, A is selected from

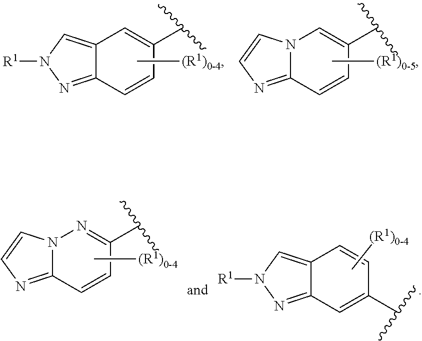

In some embodiments, A is

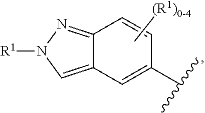

wherein R¹ is as defined herein. In some embodiments, A is selected from

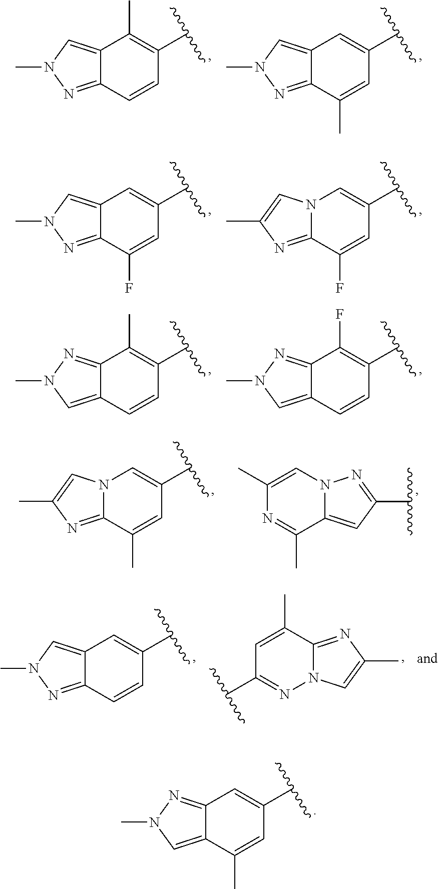

In some embodiments, A is

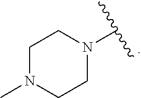 [SMILES: *N1CCN(C)CC1]

In some embodiments, A is

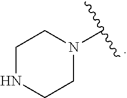 [SMILES: *N1CCNCC1]

In some embodiments, A is

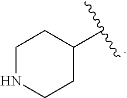 [SMILES: *C1CCNCC1]

In some embodiments, A is

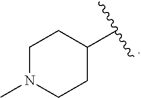 [SMILES: *C1CCN(C)CC1]

In some embodiments, A is

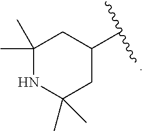 [SMILES: *C1CC(C)(C)NC(C)(C)C1]

In some embodiments, A is

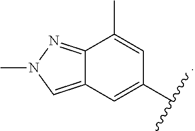

In some embodiments, A is

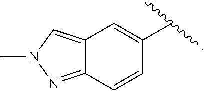

In some embodiments, A is

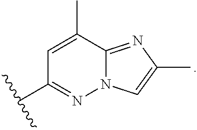

In some embodiments, A is

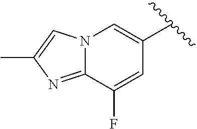 [SMILES: *c1cc(F)c2nc(C)cn2c1]

In some embodiments, B is heteroaryl. In some embodiments, B is a nitrogen-containing heteroaryl. In some embodiments, B is a bicyclic nitrogen-containing heteroaryl. In some embodiments, B is selected from

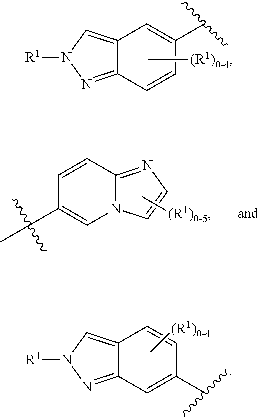

In some embodiments, B is

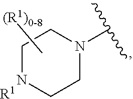

wherein R¹ is as defined herein.

In some embodiments, B is selected from

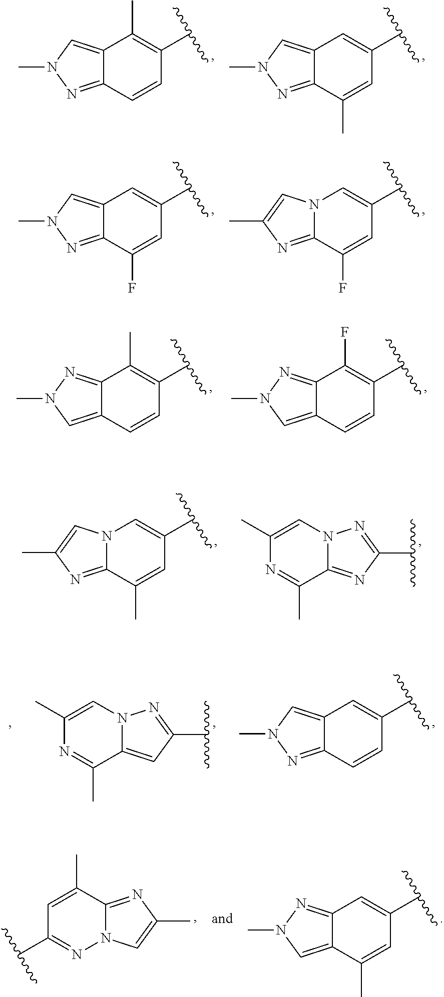

In some embodiments, B is heterocyclyl. In some embodiments, B is a nitrogen-containing heterocyclyl. In some embodiments, B is a monocyclic nitrogen-containing heterocyclyl or a bicyclic nitrogen-containing heterocyclyl. In some embodiments, B is selected from

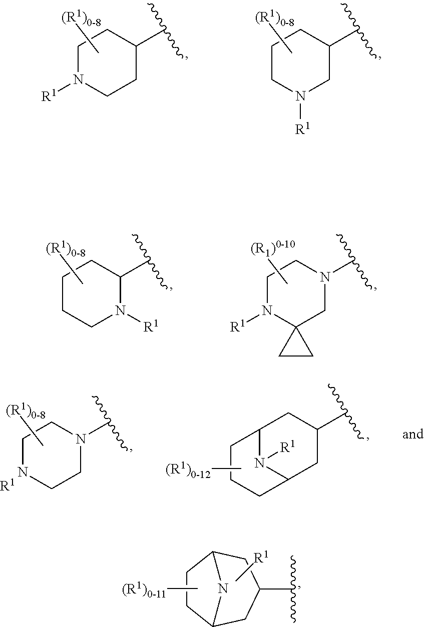

wherein R¹ is as defined herein. In some embodiments, B is selected from

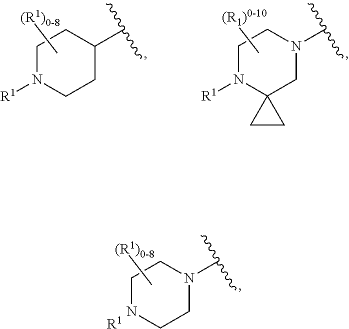

wherein R¹ is as defined herein. In some embodiments, B is selected from,

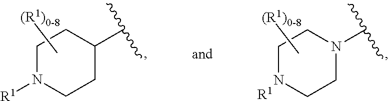

wherein R¹ is as defined herein.

In some embodiments, B is selected from

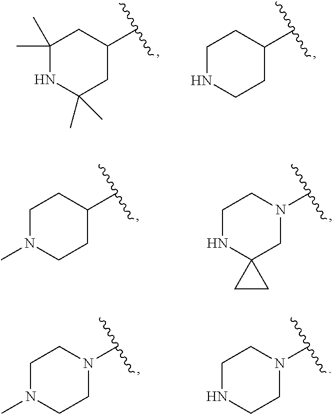

In some embodiments, B is

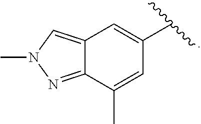 [SMILES: *c1cc(C)c2nn(C)cc2c1]

In some embodiments, B is

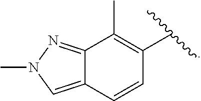 [SMILES: *c1ccc2cn(C)nc2c1C]

In some embodiments, B is

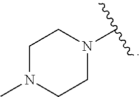 [SMILES: *N1CCN(C)CC1]

In some embodiments, B is

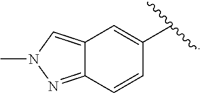 [SMILES: *c1ccc2nn(C)cc2c1]

In some embodiments, B is

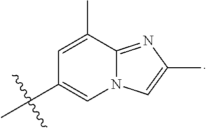 [SMILES: *c1cc(C)c2nc(C)cn2c1]

In some embodiments, B is

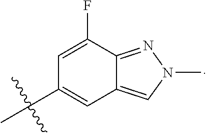 [SMILES: *c1cc(F)c2nn(C)cc2c1]

In some embodiments, B is

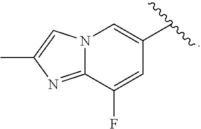 [SMILES: *c1cc(F)c2nc(C)cn2c1]

In some embodiments, B is

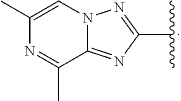 [SMILES: *c1nc2c(C)nc(C)cn2n1]

In some embodiments, B is

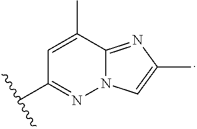 [SMILES: *c1cc(C)c2nc(C)cn2n1]

In some embodiments, B is

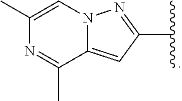 [SMILES: *c1cc2c(C)nc(C)cn2n1]

In some embodiments, B is

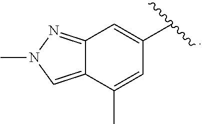 [SMILES: *c1cc(C)c2cn(C)nc2c1]

In some embodiments, B is

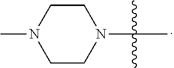 [SMILES: *N1CCN(C)CC1]

In some embodiments, B is

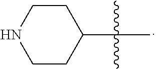 [SMILES: *C1CCNCC1]

In some embodiments, B is

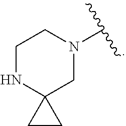 [SMILES: *N1CCNC2(CC2)C1]

In some embodiments, the compound of Formula (V) is Formula (V-b):

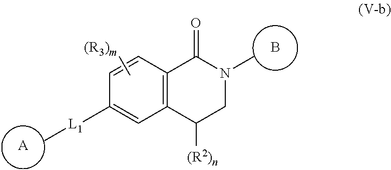

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L¹ is independently absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁷; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁵; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵; each R² is independently hydrogen or C₁-C₆-alkyl; R³ is C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —C(O)R^{D}, or —C(O)OR^{D}; R⁴ is hydrogen, C₁-C₆-alkyl, or C₁-C₆-haloalkyl; each R⁵ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁷; each R⁷ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each R^{B} and R^{C} is independently hydrogen, C₁-C₆ alkyl, C₁-C₆ heteroalkyl, cycloalkyl, heterocyclyl, —OR^{A}; or R^{B} and R^{C} together with the atom to which they are attached form a 3-7-membered heterocyclyl ring optionally substituted with one or more R⁹; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁹ is independently C₁-C₆-alkyl or halo; n is 0, 1, or 2; m is 0, 1, 2, or 3; and x is 0, 1, or 2.

In some embodiments, A is heterocyclyl optionally substituted with one or more R¹. In some embodiments, A is monocyclic nitrogen-containing heterocyclyl. In some embodiments, A is optionally substituted piperidinyl. In some embodiments, A is selected from

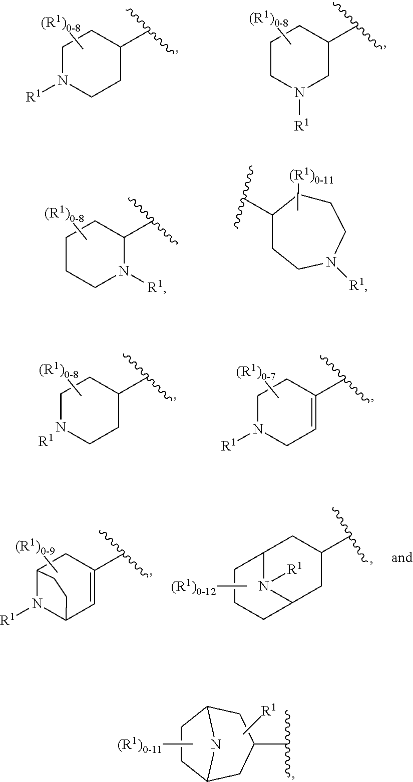

wherein R¹ is as defined herein.

In some embodiments, A is selected from,

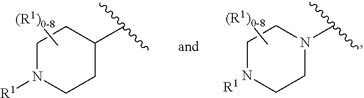

wherein R¹ is as defined herein.

In some embodiments, A is selected from

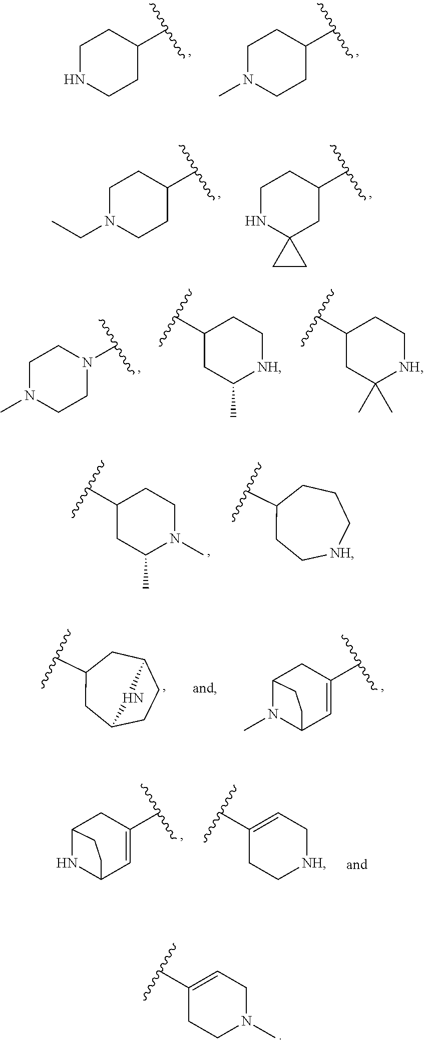

In some embodiments, A is heteroaryl. In some embodiments, A is a nitrogen-containing heteroaryl. In some embodiments, A is a bicyclic nitrogen-containing heteroaryl.

In some embodiments, A is selected from

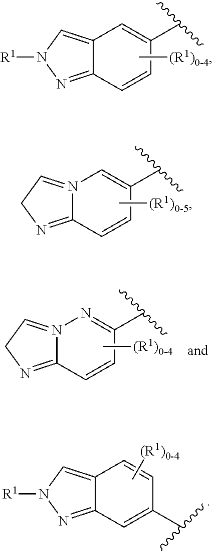

In some embodiments, A is

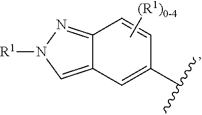

wherein R¹ is as defined herein. In some embodiments, A is selected from

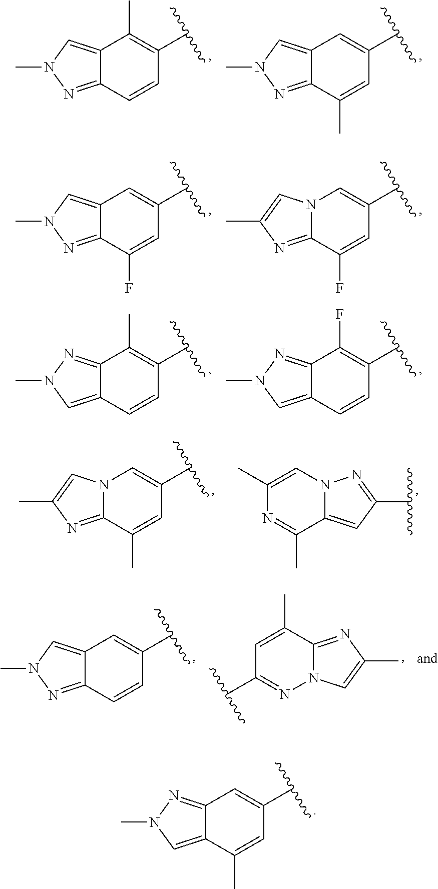

In some embodiments, A is

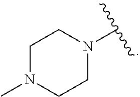 [SMILES: *N1CCN(C)CC1]

In some embodiments, A is

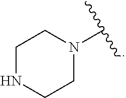 [SMILES: *N1CCNCC1]

In some embodiments, A is

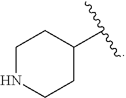 [SMILES: *C1CCNCC1]

In some embodiments, A is

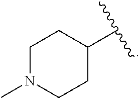 [SMILES: *C1CCN(C)CC1]

In some embodiments, A is

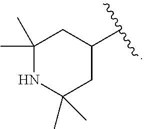 [SMILES: *C1CC(C)(C)NC(C)(C)C1]

In some embodiments, A is

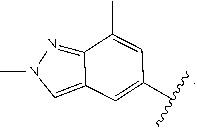 [SMILES: *c1cc(C)c2nn(C)cc2c1]

In some embodiments, A is

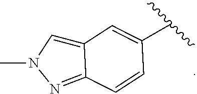 [SMILES: *c1ccc2nn(C)cc2c1]

In some embodiments, A is

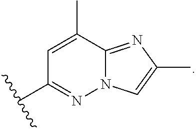 [SMILES: *c1cc(C)c2nc(C)cn2n1]

In some embodiments, A is

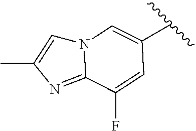 [SMILES: *c1cc(F)c2nc(C)cn2c1]

In some embodiments, B is heteroaryl. In some embodiments, B is a nitrogen-containing heteroaryl. In some embodiments, B is a bicyclic nitrogen-containing heteroaryl. In some embodiments, B is selected from

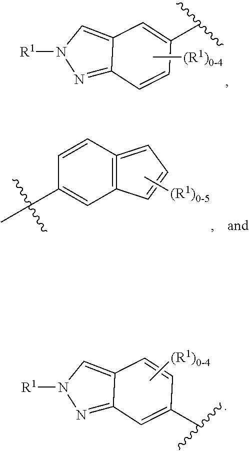

In some embodiments, B is

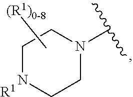

wherein R¹ is as defined herein.

In some embodiments, B is selected from

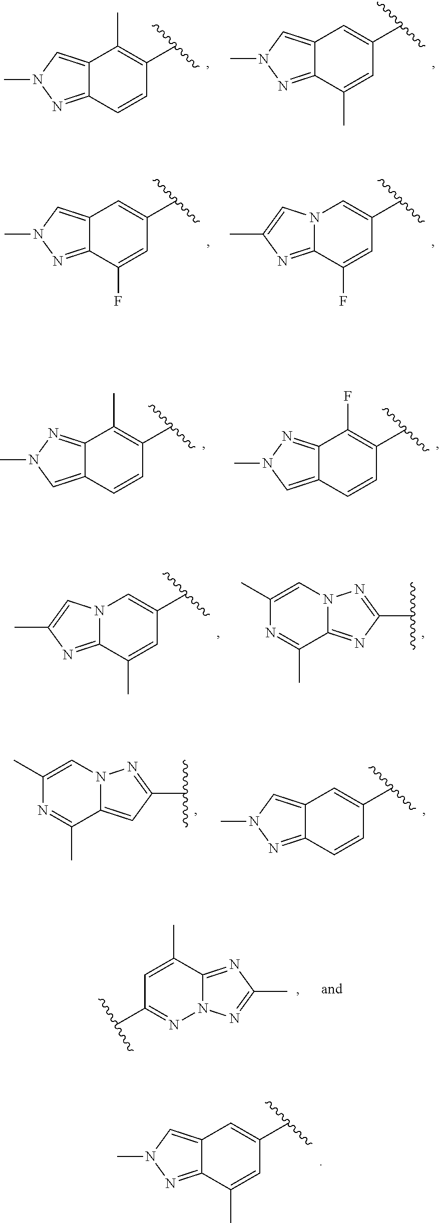

In some embodiments, B is heterocyclyl. In some embodiments, B is a nitrogen-containing heterocyclyl. In some embodiments, B is a monocyclic nitrogen-containing heterocyclyl or a bicyclic nitrogen-containing heterocyclyl. In some embodiments, B is selected from

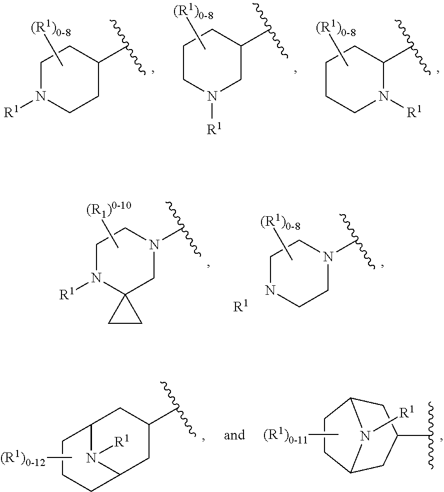

wherein R¹ is as defined herein. In some embodiments, B is selected from

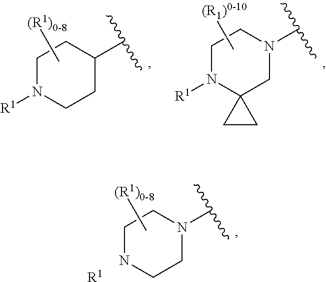

wherein R¹ is as defined herein. In some embodiments, B is selected from,

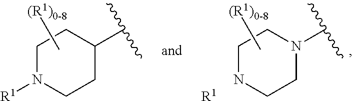

wherein R¹ is as defined herein.

In some embodiments, B is selected from

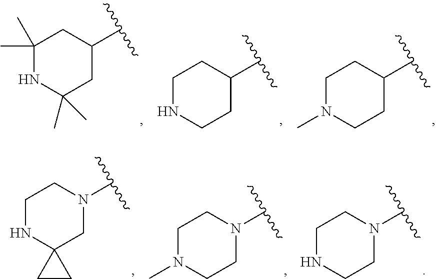

In some embodiments, B is

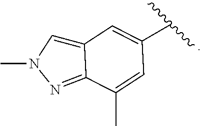 [SMILES: *c1cc(C)c2nn(C)cc2c1]

In some embodiments, B is

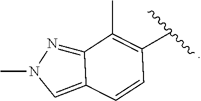 [SMILES: *c1ccc2cn(C)nc2c1C]

In some embodiments, B is

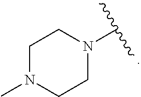 [SMILES: *N1CCN(C)CC1]

In some embodiments, B is

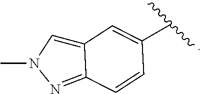 [SMILES: *c1ccc2nn(C)cc2c1]

In some embodiments, B is

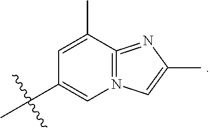

In some embodiments, B is

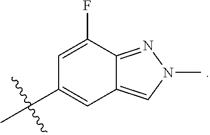 [SMILES: *c1cc(F)c2nn(C)cc2c1]

In some embodiments, B is

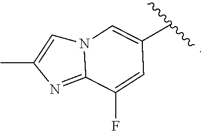 [SMILES: *c1cc(F)c2nc(C)cn2c1]

In some embodiments, B is

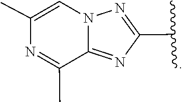 [SMILES: *c1nc2c(C)nc(C)cn2n1]

In some embodiments, B is

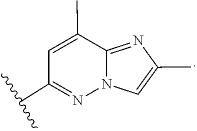 [SMILES: *c1cc(C)c2nc(C)cn2n1]

In some embodiments, B is

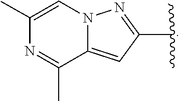 [SMILES: *c1cc2c(C)nc(C)cn2n1]

In some embodiments, B is

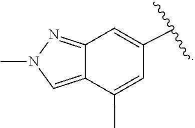 [SMILES: *c1cc(C)c2cn(C)nc2c1]

In some embodiments, B is

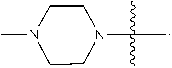 [SMILES: *N1CCN(C)CC1]

In some embodiments, B is

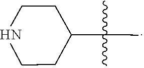 [SMILES: *C1CCNCC1]

In some embodiments, B is

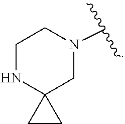 [SMILES: *N1CCNC2(CC2)C1]

In some embodiments, the compound of Formula (V) is Formula (V-c):

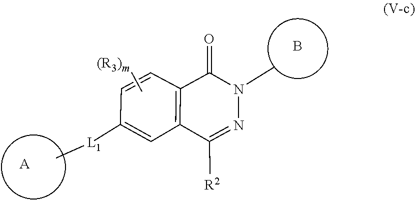

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L¹ is independently absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁷; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁵; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵; each R² is independently hydrogen or C₁-C₆-alkyl; R³ is C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —C(O)R^{D}, or —C(O)OR^{D}; R⁴ is hydrogen, C₁-C₆-alkyl, or C₁-C₆-haloalkyl; each R⁵ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C}, —NR^{B}C(O)R^{D}, NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁷; each R⁷ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each R^{B} and R^{C} is independently hydrogen, C₁-C₆ alkyl, C₁-C₆ heteroalkyl, cycloalkyl, heterocyclyl, —OR^{A}; or R^{B} and R^{C} together with the atom to which they are attached form a 3-7-membered heterocyclyl ring optionally substituted with one or more R⁹; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁹ is independently C₁-C₆-alkyl or halo; n is 0, 1, or 2; and x is 0, 1, or 2.

In some embodiments, A is heterocyclyl optionally substituted with one or more R¹. In some embodiments, A is monocyclic nitrogen-containing heterocyclyl. In some embodiments, A is optionally substituted piperidinyl. In some embodiments, A is selected from

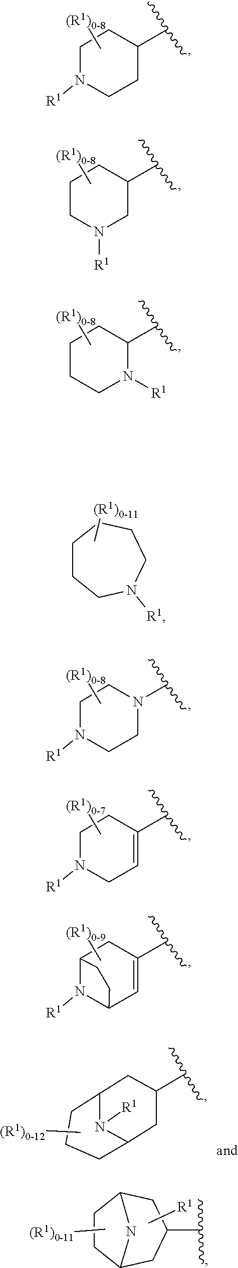

wherein R¹ is as defined herein.

In some embodiments, A is selected from,

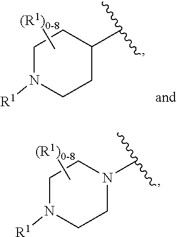

wherein R¹ is as defined herein.

In some embodiments, A is selected from

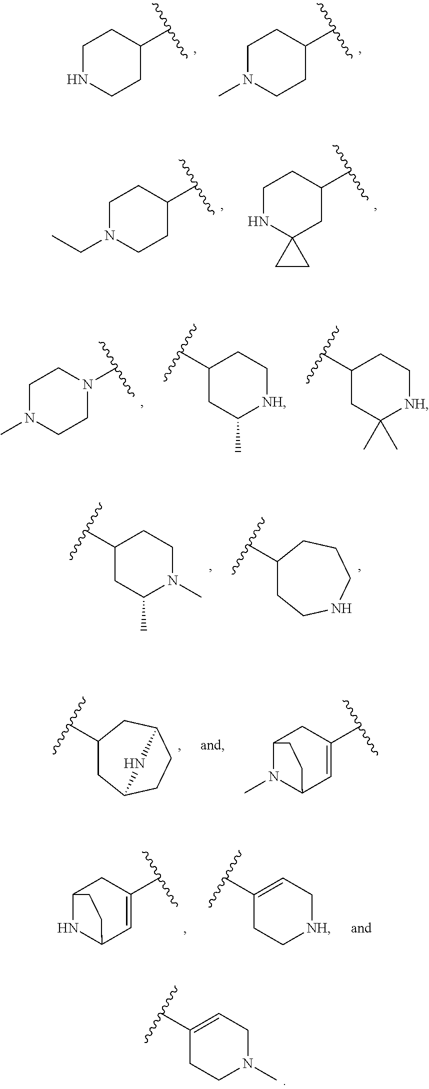

In some embodiments, A is heteroaryl. In some embodiments, A is a nitrogen-containing heteroaryl. In some embodiments, A is a bicyclic nitrogen-containing heteroaryl.

In some embodiments, A is selected from

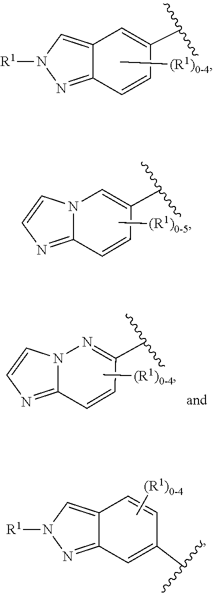

In some embodiments, A is

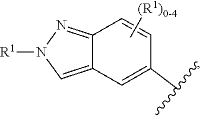

wherein R¹ is as defined herein. In some embodiments, A is selected from

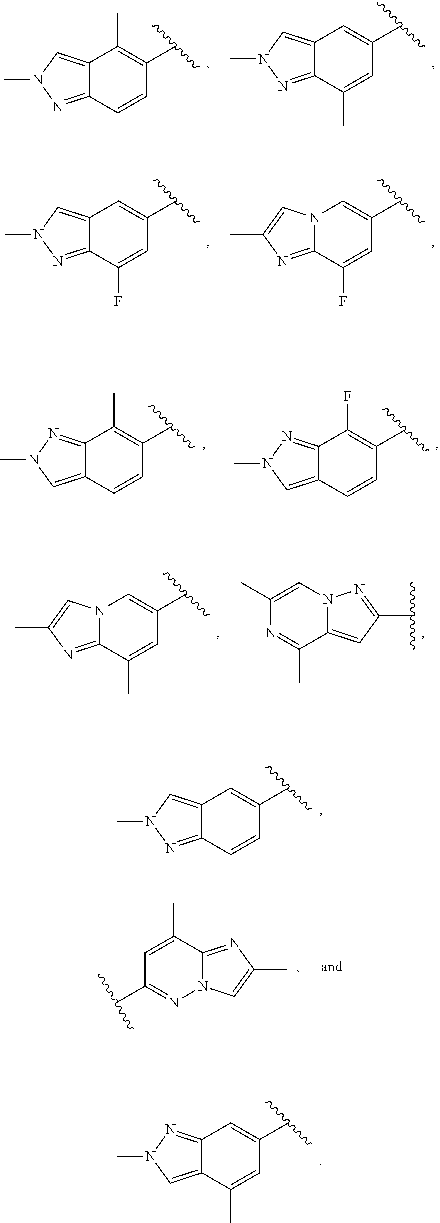

In some embodiments, A is

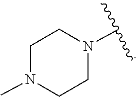 [SMILES: *N1CCN(C)CC1]

In some embodiments, A is

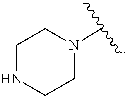 [SMILES: *N1CCNCC1]

In some embodiments, A is

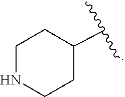 [SMILES: *C1CCNCC1]

In some embodiments, A is

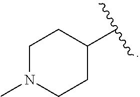 [SMILES: *C1CCN(C)CC1]

In some embodiments, A is

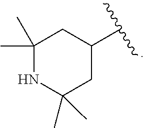 [SMILES: *C1CC(C)(C)NC(C)(C)C1]

In some embodiments, A is

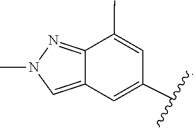 [SMILES: *c1cc(C)c2nn(C)cc2c1]

In some embodiments, A is

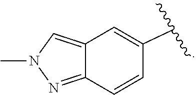 [SMILES: *c1ccc2nn(C)cc2c1]

In some embodiments, A is

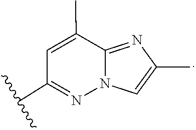 [SMILES: *c1cc(C)c2nc(C)cn2n1]

In some embodiments, A is

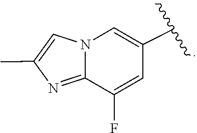 [SMILES: *c1cc(F)c2nc(C)cn2c1]

In some embodiments, B is heteroaryl. In some embodiments, B is a nitrogen-containing heteroaryl. In some embodiments, B is a bicyclic nitrogen-containing heteroaryl. In some embodiments, B is selected from

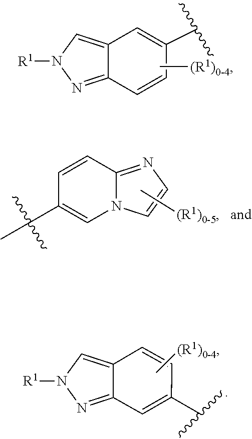

In some embodiments, B is

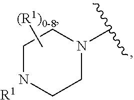

wherein R¹ is as defined herein.

In some embodiments, B is selected from

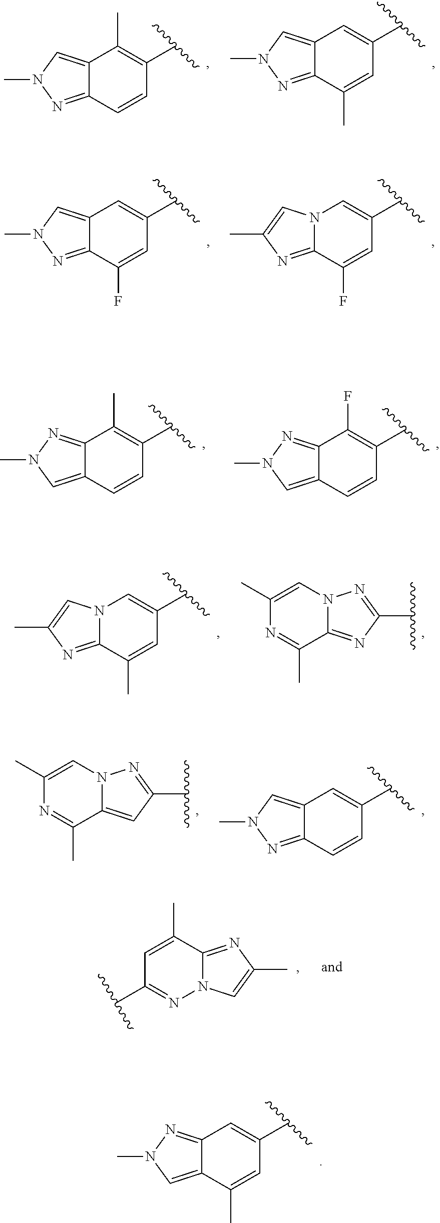

In some embodiments, B is heterocyclyl. In some embodiments, B is a nitrogen-containing heterocyclyl. In some embodiments, B is a monocyclic nitrogen-containing heterocyclyl or a bicyclic nitrogen-containing heterocyclyl. In some embodiments, B is selected from

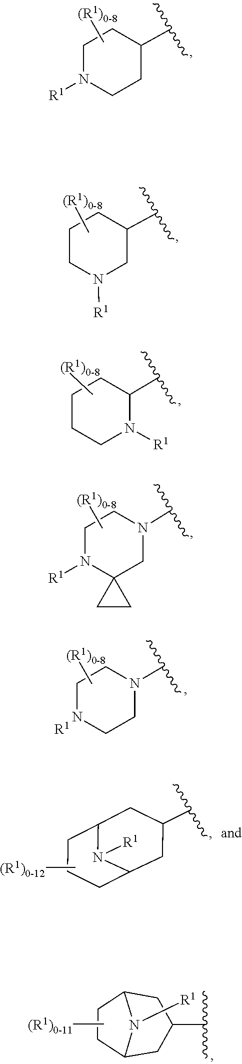

wherein R¹ is as defined herein. In some embodiments, B is selected from

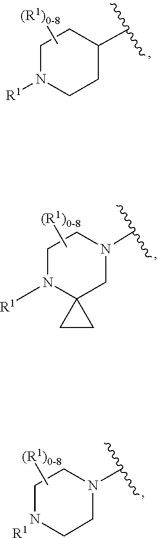

wherein R¹ is as defined herein. In some embodiments, B is selected from,

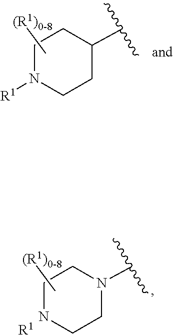

wherein R¹ is as defined herein.

In some embodiments, B is selected from

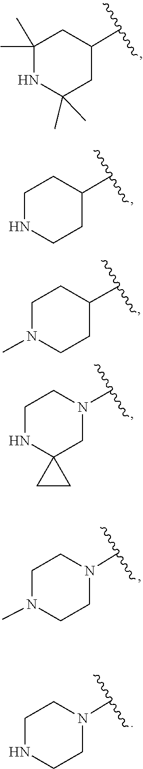

In some embodiments, B is

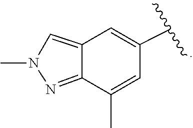

In some embodiments, B is

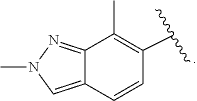 [SMILES: *c1ccc2cn(C)nc2c1C]

In some embodiments, B is

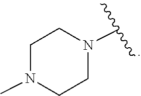 [SMILES: *N1CCN(C)CC1]

In some embodiments, B is

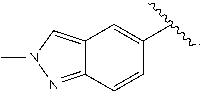 [SMILES: *c1ccc2nn(C)cc2c1]

In some embodiments, B is

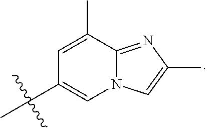 [SMILES: *c1cc(C)c2nc(C)cn2c1]

In some embodiments, B is

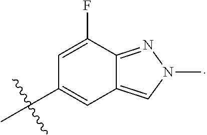 [SMILES: *c1cc(F)c2nn(C)cc2c1]

In some embodiments, B is

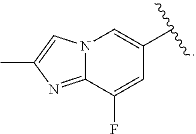 [SMILES: *c1cc(F)c2nc(C)cn2c1]

In some embodiments, B is

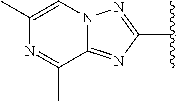 [SMILES: *c1nc2c(C)nc(C)cn2n1]

In some embodiments, B is

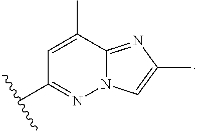 [SMILES: *c1cc(C)c2nc(C)cn2n1]

In some embodiments, B is

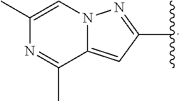 [SMILES: *c1cc2c(C)nc(C)cn2n1]

In some embodiments, B is

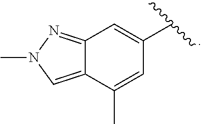 [SMILES: *c1cc(C)c2cn(C)nc2c1]

In some embodiments, B is

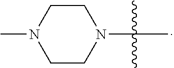 [SMILES: *N1CCN(C)CC1]

In some embodiments, B is

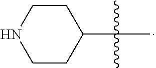 [SMILES: *C1CCNCC1]

In some embodiments, B is

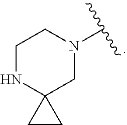 [SMILES: *N1CCNC2(CC2)C1]

In some embodiments, the compound of Formula (V) is Formula (V-d):

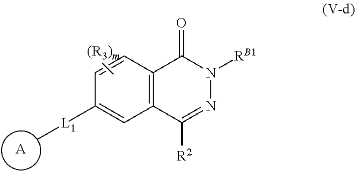

or a pharmaceutically acceptable salt, solvate, hydrate, tautomer, or stereoisomer thereof, wherein A is cycloalkyl, heterocyclyl, aryl, or heteroaryl, each of which is optionally substituted with one or more R¹; R^{B1} is C₁-C₆-alkyl or C₁-C₆-heteroalkyl, each of which is optionally substituted with R¹⁰; L¹ is independently absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁷; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁵; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵; each R² is independently hydrogen or C₁-C₆-alkyl; R³ is C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —C(O)R^{D}, or —C(O)OR^{D}; R⁴ is hydrogen, C₁-C₆-alkyl, or C₁-C₆-haloalkyl; each R⁵ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C}, —NR^{B}C(O)R^{D}, —NO₂, —C(O)NR^{B}R^{C}, —C(O)R^{D}, C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁷; each R⁷ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each R^{B} and R^{C} is independently hydrogen, C₁-C₆ alkyl, C₁-C₆ heteroalkyl, cycloalkyl, heterocyclyl, —OR^{A}; or R^{B} and R^{C} together with the atom to which they are attached form a 3-7-membered heterocyclyl ring optionally substituted with one or more R⁹; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁹ and R¹⁰ is independently C₁-C₆-alkyl or halo; n is 0, 1, or 2; and x is 0, 1, or 2.

In some embodiments, A is heterocyclyl optionally substituted with one or more R¹. In some embodiments, A is monocyclic nitrogen-containing heterocyclyl. In some embodiments, A is optionally substituted piperidinyl. In some embodiments, A is selected from

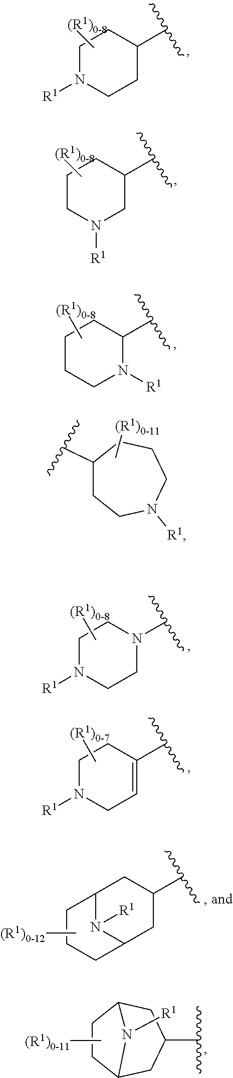

wherein R¹ is as defined herein.

In some embodiments, A is selected from,

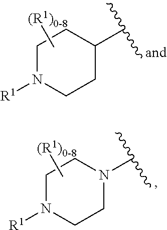

wherein R¹ is as defined herein.

In some embodiments, A is selected from

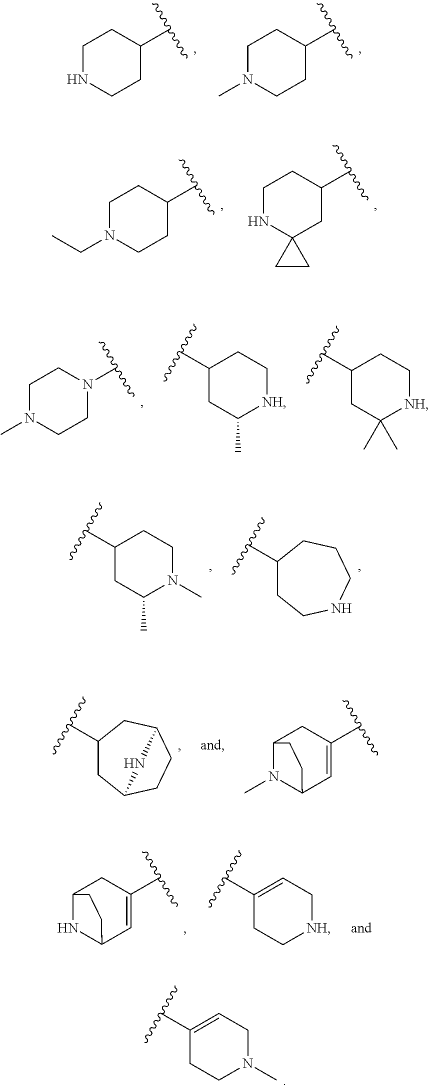

In some embodiments, A is heteroaryl. In some embodiments, A is a nitrogen-containing heteroaryl. In some embodiments, A is a bicyclic nitrogen-containing heteroaryl.

In some embodiments, A is selected from

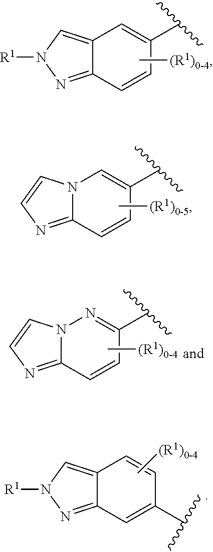

In some embodiments, A is

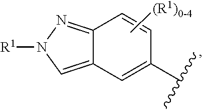

wherein R¹ is as defined herein. In some embodiments, A is selected from

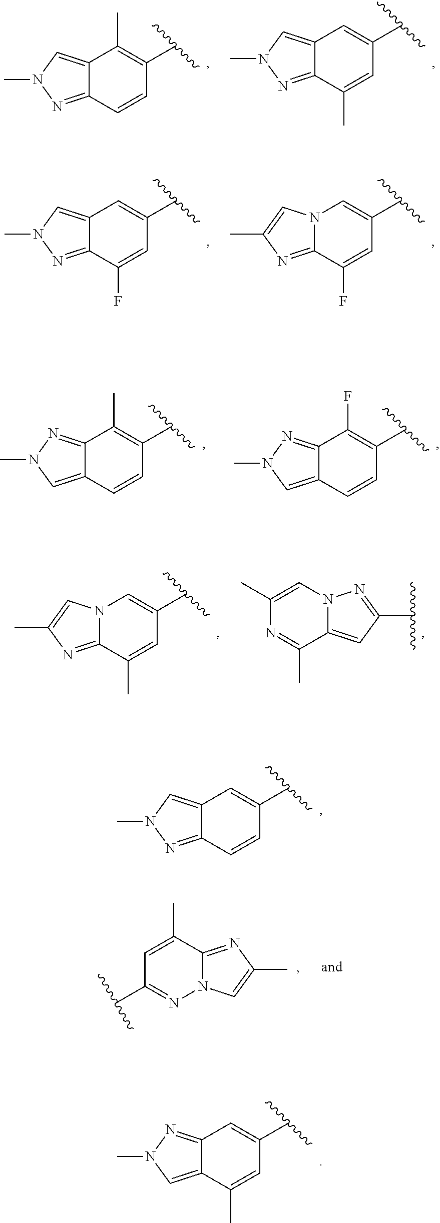

In some embodiments, A is

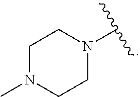 [SMILES: *N1CCN(C)CC1]

In some embodiments, A is

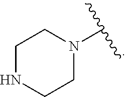 [SMILES: *N1CCNCC1]

In some embodiments, A is

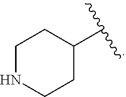 [SMILES: *C1CCNCC1]

In some embodiments, A is

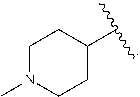 [SMILES: *C1CCN(C)CC1]

In some embodiments, A is

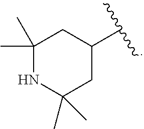 [SMILES: *C1CC(C)(C)NC(C)(C)C1]

In some embodiments, A is

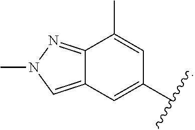 [SMILES: *c1cc(C)c2nn(C)cc2c1]

In some embodiments, A is

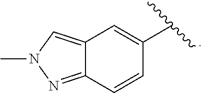 [SMILES: *c1ccc2nn(C)cc2c1]

In some embodiments, A is

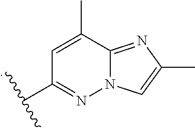 [SMILES: *c1cc(C)c2nc(C)cn2n1]

In some embodiments, A is

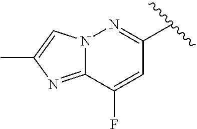 [SMILES: *c1cc(F)c2nc(C)cn2n1]

In some embodiments, B is heteroaryl. In some embodiments, B is a nitrogen-containing heteroaryl. In some embodiments, B is a bicyclic nitrogen-containing heteroaryl. In some embodiments, B is selected from

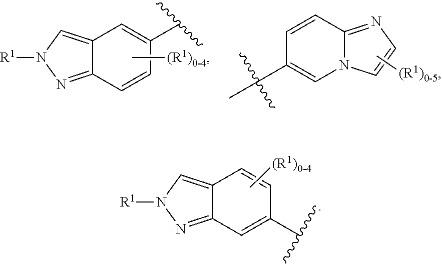

In some embodiments, B is

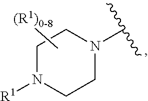

wherein R¹ is as defined herein.

In some embodiments, B is selected from

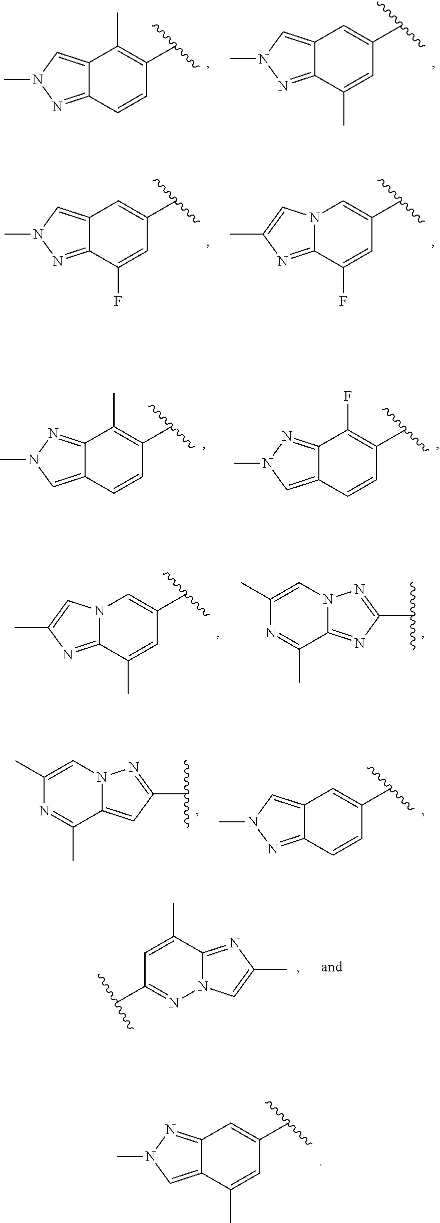

In some embodiments, B is heterocyclyl. In some embodiments, B is a nitrogen-containing heterocyclyl. In some embodiments, B is a monocyclic nitrogen-containing heterocyclyl or a bicyclic nitrogen-containing heterocyclyl. In some embodiments, B is selected from

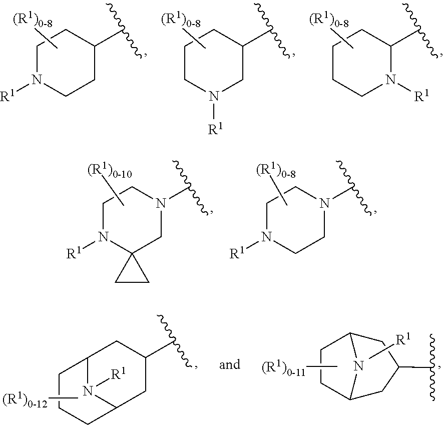

wherein R¹ is as defined herein. In some embodiments, B is selected from

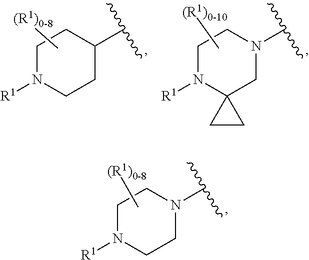

wherein R¹ is as defined herein. In some embodiments, B is selected from,

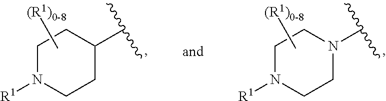

wherein R¹ is as defined herein.

In some embodiments, B is selected from

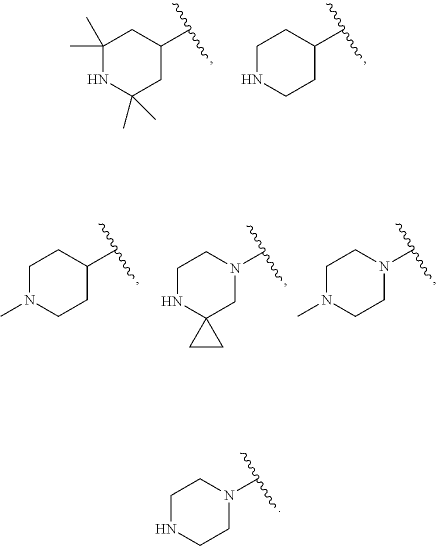

In some embodiments, B is

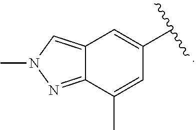 [SMILES: *c1cc(C)c2nn(C)cc2c1]

In some embodiments, B is

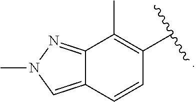 [SMILES: *c1ccc2cn(C)nc2c1C]

In some embodiments, B is

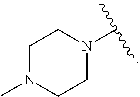 [SMILES: *N1CCN(C)CC1]

In some embodiments, B is

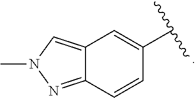 [SMILES: *c1ccc2nn(C)cc2c1]

In some embodiments, B is

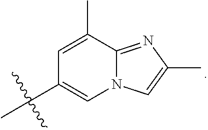 [SMILES: *c1cc(C)c2nc(C)cn2c1]

In some embodiments, B is

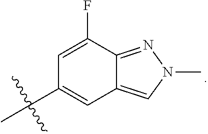 [SMILES: *c1cc(F)c2nn(C)cc2c1]

In some embodiments, B is

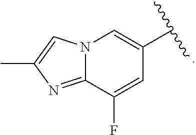 [SMILES: *c1cc(F)c2nc(C)cn2c1]

In some embodiments, B is

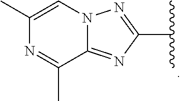 [SMILES: *c1nc2c(C)nc(C)cn2n1]

In some embodiments, B is

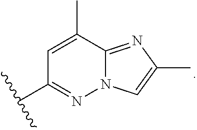 [SMILES: *c1cc(C)c2nc(C)cn2n1]

In some embodiments, B is

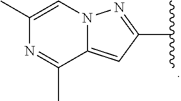 [SMILES: *c1cc2c(C)nc(C)cn2n1]

In some embodiments, B is

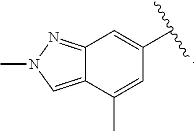 [SMILES: *c1cc(C)c2cn(C)nc2c1]

In some embodiments, B is

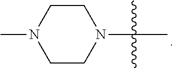 [SMILES: *N1CCN(C)CC1]

In some embodiments, B is

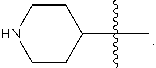 [SMILES: *C1CCNCC1]

In some embodiments, B is

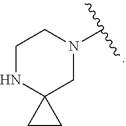 [SMILES: *N1CCNC2(CC2)C1]

In some embodiments, the compound of Formula (V) is selected from a compound in Table 5, or a pharmaceutically acceptable salt, solvate, hydrate, tautomer, or stereoisomer thereof.

TABLE 5
Exemplary compounds of Formula (V)

| Compound | |
|---|---|
| No. | Structure |
| 185 | 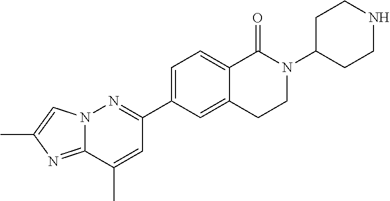 [SMILES: Cc1cn2nc(-c3ccc4c(c3)CCN(C3CCNCC3)C4=O)cc(C)c2n1] |
| 186 | 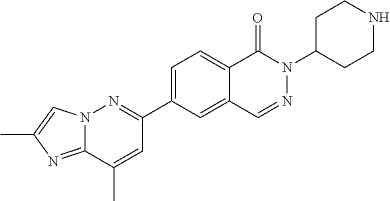 [SMILES: Cc1cn2nc(-c3ccc4c(=O)n(C5CCNCC5)ncc4c3)cc(C)c2n1] |
| 187 | 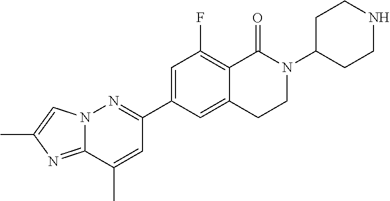 [SMILES: Cc1cn2nc(-c3cc(F)c4c(c3)CCN(C3CCNCC3)C4=O)cc(C)c2n1] |
| 188 | 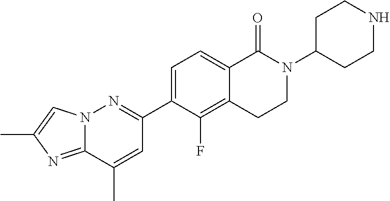 [SMILES: Cc1cn2nc(-c3ccc4c(c3F)CCN(C3CCNCC3)C4=O)cc(C)c2n1] |
| 215 | 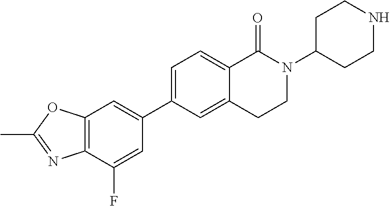 [SMILES: Cc1nc2c(F)cc(-c3ccc4c(c3)CCN(C3CCNCC3)C4=O)cc2o1] |
| 216 | 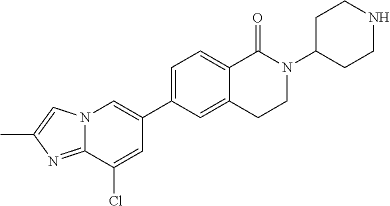 [SMILES: Cc1cn2cc(-c3ccc4c(c3)CCN(C3CCNCC3)C4=O)cc(Cl)c2n1] |
| 217 | 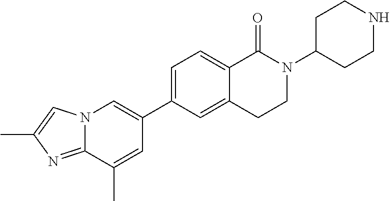 [SMILES: Cc1cn2cc(-c3ccc4c(c3)CCN(C3CCNCC3)C4=O)cc(C)c2n1] |
| 218 | 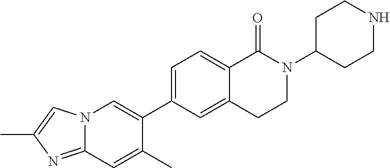 [SMILES: Cc1cn2cc(-c3ccc4c(c3)CCN(C3CCNCC3)C4=O)c(C)cc2n1] |
| 219 | 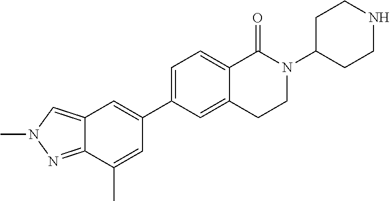 [SMILES: Cc1cc(-c2ccc3c(c2)CCN(C2CCNCC2)C3=O)cc2cn(C)nc12] |
| 220 | 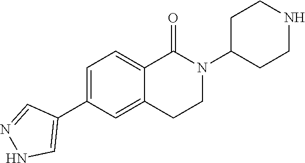 [SMILES: O=C1c2ccc(-c3cn[nH]c3)cc2CCN1C1CCNCC1] |
| 221 | 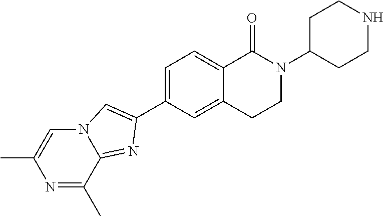 [SMILES: Cc1cn2cc(-c3ccc4c(c3)CCN(C3CCNCC3)C4=O)nc2c(C)n1] |
| 222 | 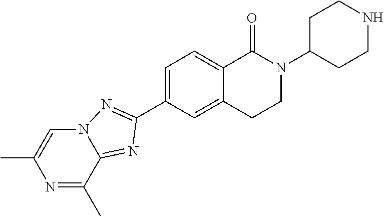 [SMILES: Cc1cn2nc(-c3ccc4c(c3)CCN(C3CCNCC3)C4=O)nc2c(C)n1] |
| 223 | 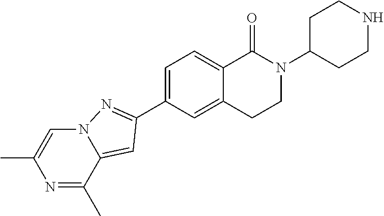 [SMILES: Cc1cn2nc(-c3ccc4c(c3)CCN(C3CCNCC3)C4=O)cc2c(C)n1] |
| 224 | 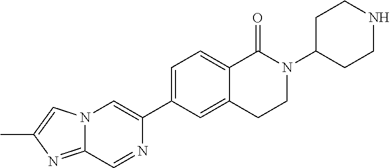 [SMILES: Cc1cn2cc(-c3ccc4c(c3)CCN(C3CCNCC3)C4=O)ncc2n1] |
| 225 | 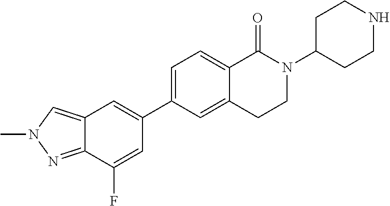 [SMILES: Cn1cc2cc(-c3ccc4c(c3)CCN(C3CCNCC3)C4=O)cc(F)c2n1] |
| 226 | 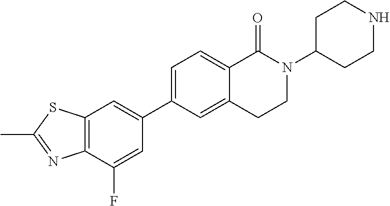 [SMILES: Cc1nc2c(F)cc(-c3ccc4c(c3)CCN(C3CCNCC3)C4=O)cc2s1] |
| 247 | 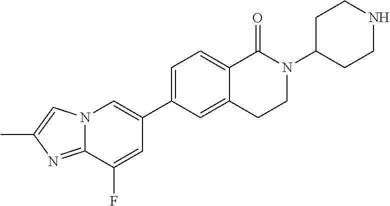 [SMILES: Cc1cn2cc(-c3ccc4c(c3)CCN(C3CCNCC3)C4=O)cc(F)c2n1] |
| 248 | 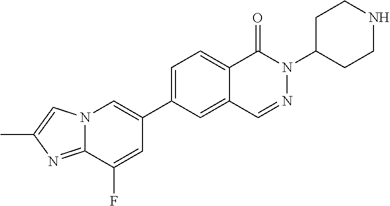 [SMILES: Cc1cn2cc(-c3ccc4c(=O)n(C5CCNCC5)ncc4c3)cc(F)c2n1] |
| 249 | 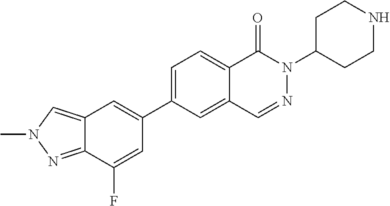 [SMILES: Cn1cc2cc(-c3ccc4c(=O)n(C5CCNCC5)ncc4c3)cc(F)c2n1] |
| 250 | 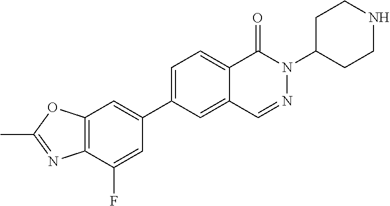 [SMILES: Cc1nc2c(F)cc(-c3ccc4c(=O)n(C5CCNCC5)ncc4c3)cc2o1] |
| 251 | 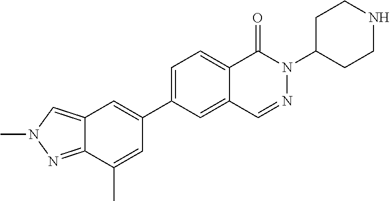 [SMILES: Cc1cc(-c2ccc3c(=O)n(C4CCNCC4)ncc3c2)cc2cn(C)nc12] |
| 252 | 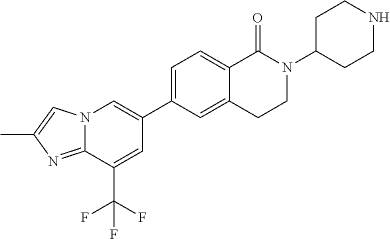 [SMILES: Cc1cn2cc(-c3ccc4c(c3)CCN(C3CCNCC3)C4=O)cc(C(F)(F)F)c2n1] |
| 253 | 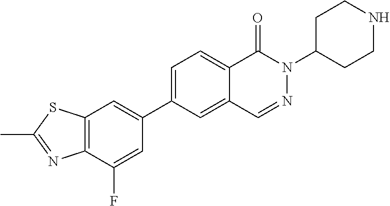 [SMILES: Cc1nc2c(F)cc(-c3ccc4c(=O)n(C5CCNCC5)ncc4c3)cc2s1] |
| 254 | 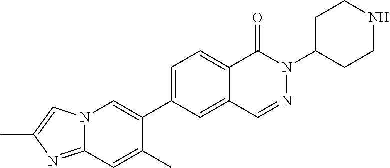 [SMILES: Cc1cn2cc(-c3ccc4c(=O)n(C5CCNCC5)ncc4c3)c(C)cc2n1] |
| 255 | 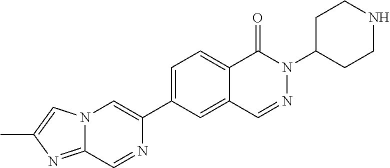 [SMILES: Cc1cn2cc(-c3ccc4c(=O)n(C5CCNCC5)ncc4c3)ncc2n1] |
| 256 | 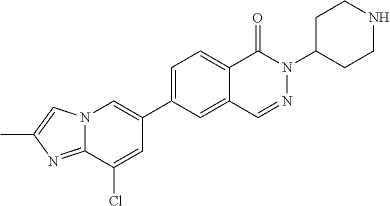 [SMILES: Cc1cn2cc(-c3ccc4c(=O)n(C5CCNCC5)ncc4c3)cc(Cl)c2n1] |
| 257 | 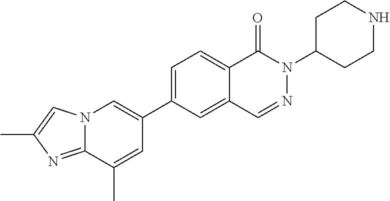 [SMILES: Cc1cn2cc(-c3ccc4c(=O)n(C5CCNCC5)ncc4c3)cc(C)c2n1] |
| 258 | 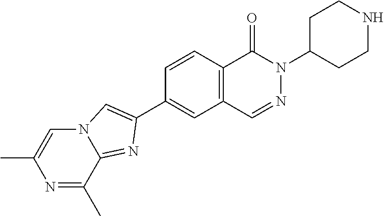 [SMILES: Cc1cn2cc(-c3ccc4c(=O)n(C5CCNCC5)ncc4c3)nc2c(C)n1] |
| 259 | 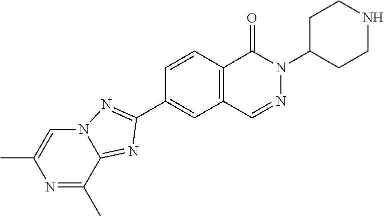 [SMILES: Cc1cn2nc(-c3ccc4c(=O)n(C5CCNCC5)ncc4c3)nc2c(C)n1] |
| 260 | 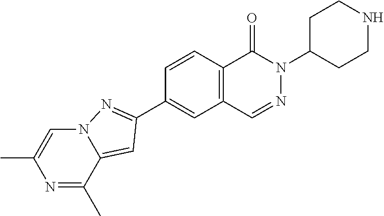 [SMILES: Cc1cn2nc(-c3ccc4c(=O)n(C5CCNCC5)ncc4c3)cc2c(C)n1] |
| 261 | 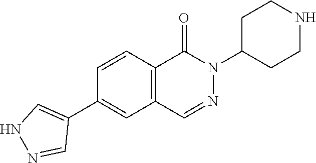 [SMILES: O=c1c2ccc(-c3cn[nH]c3)cc2cnn1C1CCNCC1] |
| 262 | 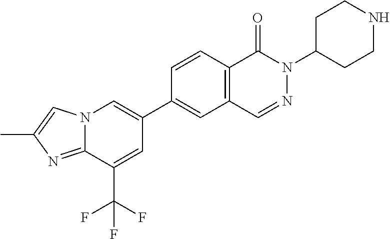 [SMILES: Cc1cn2cc(-c3ccc4c(=O)n(C5CCNCC5)ncc4c3)cc(C(F)(F)F)c2n1] |
| 263 | 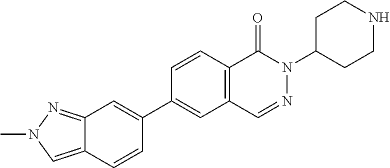 [SMILES: Cn1cc2ccc(-c3ccc4c(=O)n(C5CCNCC5)ncc4c3)cc2n1] |
| 264 | 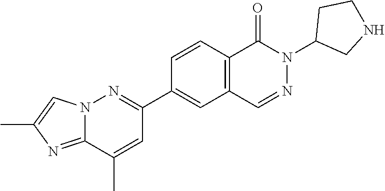 [SMILES: Cc1cn2nc(-c3ccc4c(=O)n(C5CCNC5)ncc4c3)cc(C)c2n1] |
| 265 | 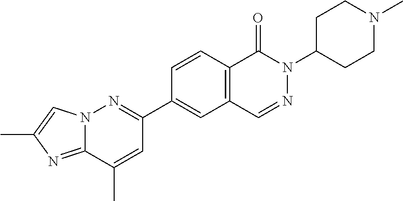 |
| 266 | 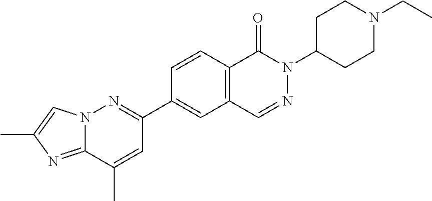 [SMILES: CCN1CCC(n2ncc3cc(-c4cc(C)c5nc(C)cn5n4)ccc3c2=O)CC1] |
| 267 | 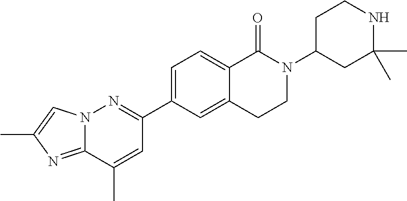 |
| 268 | 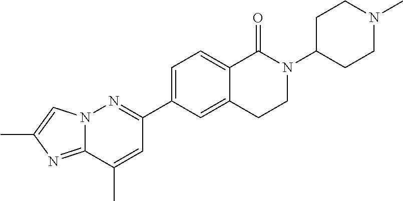 |
| 269 | 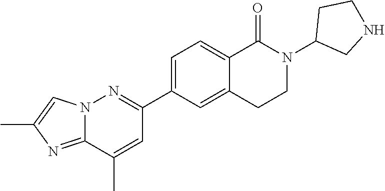 [SMILES: Cc1cn2nc(-c3ccc4c(c3)CCN(C3CCNC3)C4=O)cc(C)c2n1] |
| 270 | 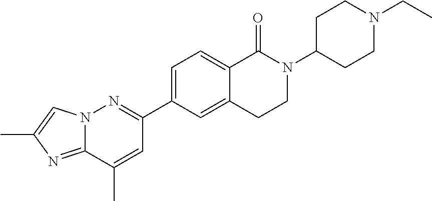 [SMILES: CCN1CCC(N2CCc3cc(-c4cc(C)c5nc(C)cn5n4)ccc3C2=O)CC1] |
| 271 | 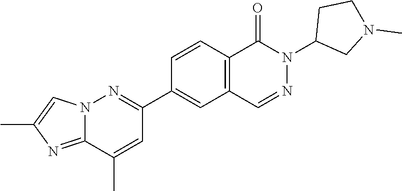 |
| 272 | 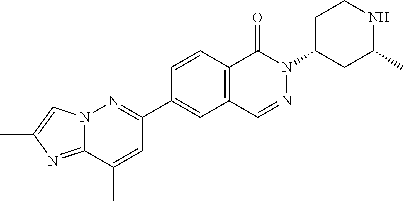 |
| 273 | 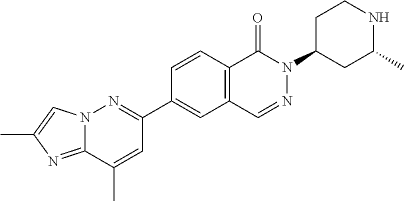 |
| 274 | 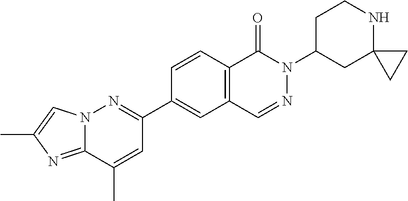 [SMILES: Cc1cn2nc(-c3ccc4c(=O)n(C5CCNC6(CC6)C5)ncc4c3)cc(C)c2n1] |
| 275 | 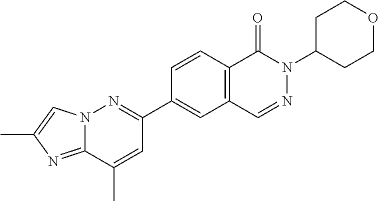 [SMILES: Cc1cn2nc(-c3ccc4c(=O)n(C5CCOCC5)ncc4c3)cc(C)c2n1] |
| 276 | 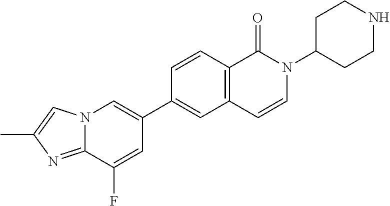 [SMILES: Cc1cn2cc(-c3ccc4c(=O)n(C5CCNCC5)ccc4c3)cc(F)c2n1] |
| 277 | 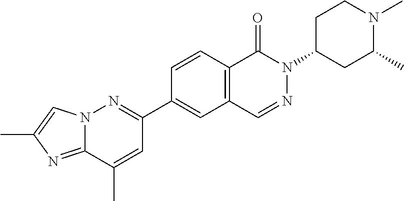 |
| 278 | 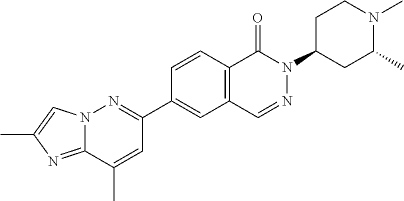 |
| 279 | 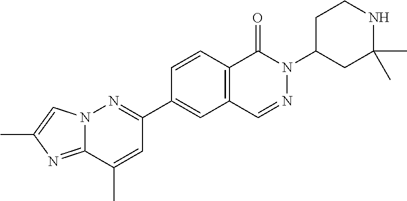 |
| 280 | 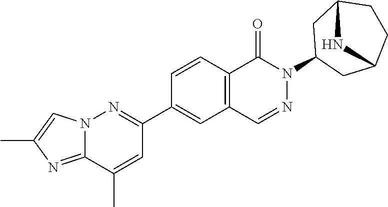 [SMILES: Cc1cn2nc(-c3ccc4c(=O)n([C@@H]5C[C@H]6CC[C@@H](C5)N6)ncc4c3)cc(C)c2n1] |
| 281 | 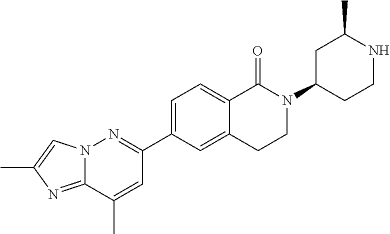 [SMILES: Cc1cn2nc(-c3ccc4c(c3)CCN([C@@H]3CCN[C@H](C)C3)C4=O)cc(C)c2n1] |
| 282 | 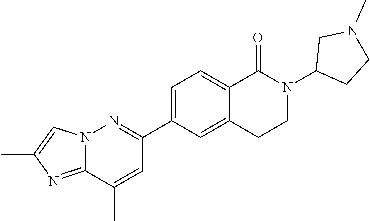 [SMILES: Cc1cn2nc(-c3ccc4c(c3)CCN(C3CCN(C)C3)C4=O)cc(C)c2n1] |
| 283 | 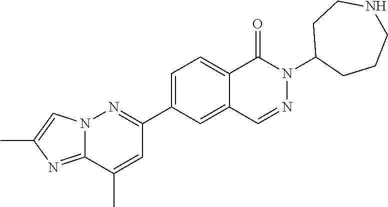 [SMILES: Cc1cn2nc(-c3ccc4c(=O)n(C5CCCNCC5)ncc4c3)cc(C)c2n1] |
| 304 | 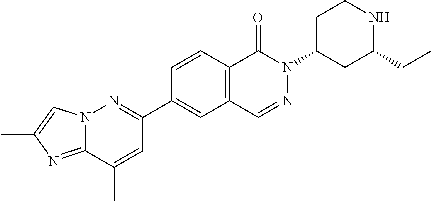 [SMILES: CC[C@@H]1C[C@H](n2ncc3cc(-c4cc(C)c5nc(C)cn5n4)ccc3c2=O)CCN1] |
| 305 | 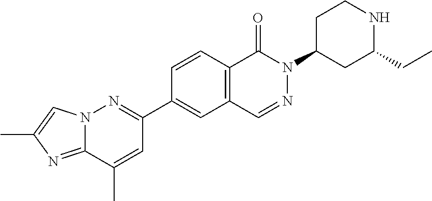 [SMILES: CC[C@@H]1C[C@@H](n2ncc3cc(-c4cc(C)c5nc(C)cn5n4)ccc3c2=O)CCN1] |
| 309 | 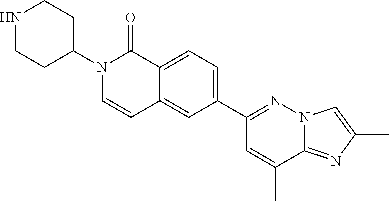 [SMILES: Cc1cn2nc(-c3ccc4c(=O)n(C5CCNCC5)ccc4c3)cc(C)c2n1] |
| 310 | 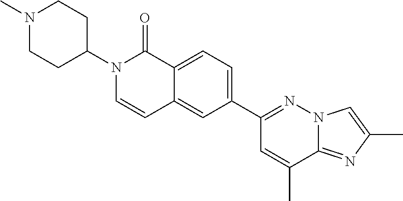 |
| 312 | 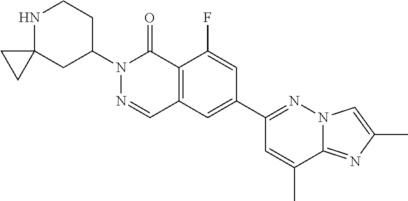 [SMILES: Cc1cn2nc(-c3cc(F)c4c(=O)n(C5CCNC6(CC6)C5)ncc4c3)cc(C)c2n1] |
| 313 | 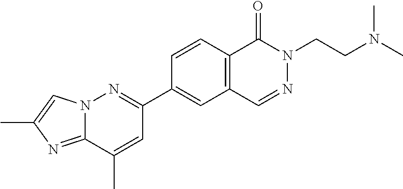 |
| 314 | 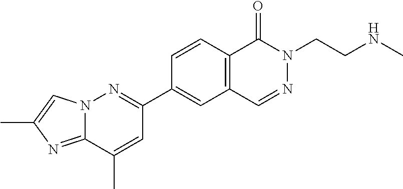 |
| 322 | 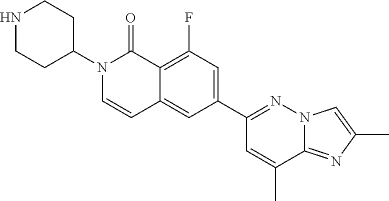 [SMILES: Cc1cn2nc(-c3cc(F)c4c(=O)n(C5CCNCC5)ccc4c3)cc(C)c2n1] |
| 324 | 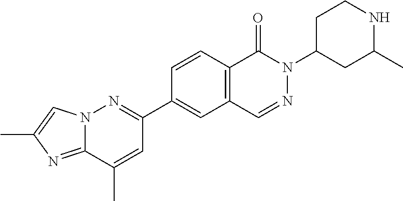 |
| 325 | 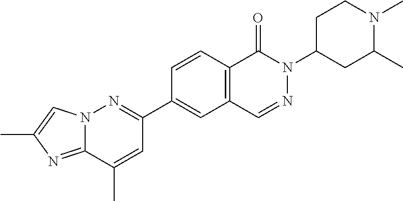 |
| 326 | 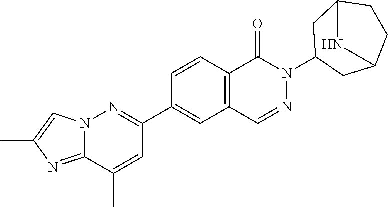 [SMILES: Cc1cn2nc(-c3ccc4c(=O)n(C5CC6CCC(C5)N6)ncc4c3)cc(C)c2n1] |
| 327 | 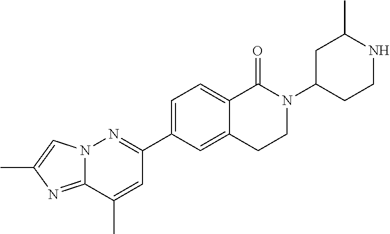 [SMILES: Cc1cn2nc(-c3ccc4c(c3)CCN(C3CCNC(C)C3)C4=O)cc(C)c2n1] |
| 328 | 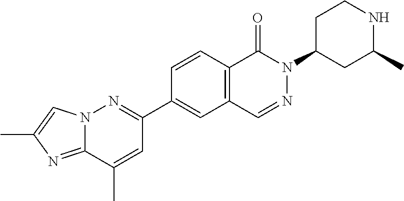 |
| 329 | 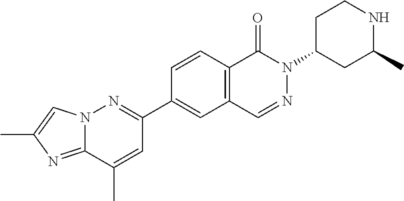 |
| 330 | 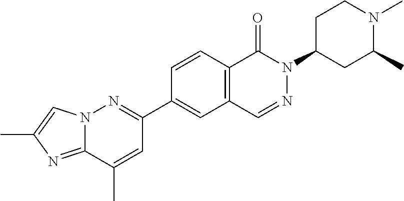 |
| 331 | 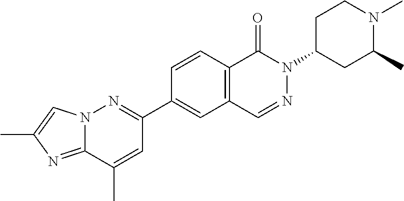 |
| 332 | 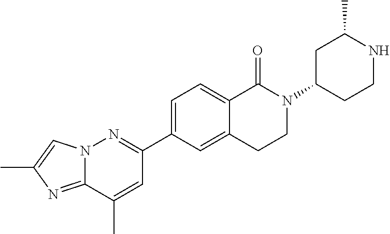 [SMILES: Cc1cn2nc(-c3ccc4c(c3)CCN([C@H]3CCN[C@@H](C)C3)C4=O)cc(C)c2n1] |
| 333 | 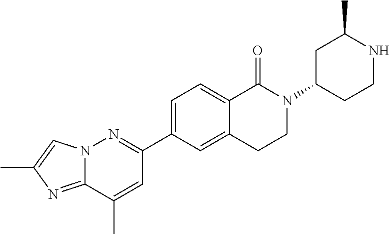 [SMILES: Cc1cn2nc(-c3ccc4c(c3)CCN([C@H]3CCN[C@H](C)C3)C4=O)cc(C)c2n1] |
| 334 | 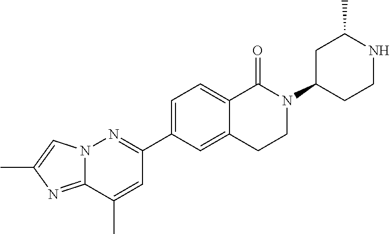 [SMILES: Cc1cn2nc(-c3ccc4c(c3)CCN([C@@H]3CCN[C@@H](C)C3)C4=O)cc(C)c2n1] |
| 335 | 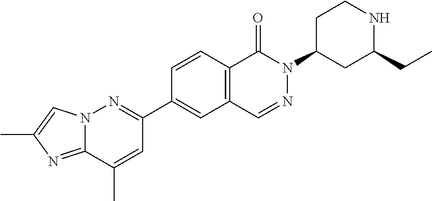 [SMILES: CC[C@H]1C[C@@H](n2ncc3cc(-c4cc(C)c5nc(C)cn5n4)ccc3c2=O)CCN1] |
| 336 | 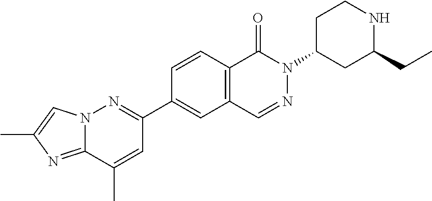 [SMILES: CC[C@H]1C[C@H](n2ncc3cc(-c4cc(C)c5nc(C)cn5n4)ccc3c2=O)CCN1] |

In some embodiments, for Formula (V), A is bicyclic heterocyclyl (e.g., 2,8-dimethylimidazo[1,2-b]pyridazyl); B is monocyclic heterocyclyl (e.g., piperidinyl); L¹ and L² are absent; Y is C(R^{6a})(R^{6b}) (e.g., CH₂); each R² is hydrogen; m is 0; and n is 2. In some embodiments, the compound of Formula (V) is Compound 185, or a pharmaceutically acceptable salt, solvate, hydrate, tautomer, or stereoisomer thereof.

In some embodiments, for Formula (V), A is bicyclic heterocyclyl (e.g., 2,8-dimethylimidazo[1,2-b]pyridazyl); B is monocyclic heterocyclyl (e.g., piperidinyl); L¹ and L² are absent; Y is N; R² is hydrogen; m is 0; and n is 1. In some embodiments, the compound of Formula (V) is Compound 186, or a pharmaceutically acceptable salt, solvate, hydrate, tautomer, or stereoisomer thereof.

In some embodiments, for Formula (V), A is bicyclic heterocyclyl (e.g., 2,8-dimethylimidazo[1,2-b]pyridazyl); B is monocyclic heterocyclyl (e.g., piperidinyl); L¹ and L² are absent; Y is C(R^{6a})(R^{6b}) (e.g., CH₂); each R² is hydrogen; R³ is halo (e.g., F); m is 1; and n is 2. In some embodiments, the compound of Formula (V) is Compound 187, 188, or a pharmaceutically acceptable salt, solvate, hydrate, tautomer, or stereoisomer thereof.

In some embodiments, for Formula (V), A is bicyclic heterocyclyl (e.g., 4-fluoro-2-methylbenzo[d]oxazolyl); B is monocyclic heterocyclyl (e.g., piperidinyl); L¹ and L² are absent; Y is C(R^{6a})(R^{6b}) (e.g., CH₂); each R² is hydrogen; m is 0; and n is 2. In some embodiments, the compound of Formula (V) is Compound 215, or a pharmaceutically acceptable salt, solvate, hydrate, tautomer, or stereoisomer thereof.

In some embodiments, for Formula (V), A is bicyclic heterocyclyl (e.g., 8-chloro-2-methylimidazo[1,2-a]pyridinyl); B is monocyclic heterocyclyl (e.g., piperidinyl); L¹ and L² are absent; Y is C(R^{6a})(R^{6b}) (e.g., CH₂); each R² is hydrogen; m is 0; and n is 2. In some embodiments, the compound of Formula (V) is Compound 216, or a pharmaceutically acceptable salt, solvate, hydrate, tautomer, or stereoisomer thereof.

In some embodiments, for Formula (V), A is bicyclic heterocyclyl (e.g., 2,8-dimethylimidazo[1,2-a]pyridinyl); B is monocyclic heterocyclyl (e.g., piperidinyl); L¹ and L² are absent; Y is C(R^{6a})(R^{6b}) (e.g., CH₂); each R² is hydrogen; m is 0; and n is 2. In some embodiments, the compound of Formula (V) is Compound 217, or a pharmaceutically acceptable salt, solvate, hydrate, tautomer, or stereoisomer thereof.

In some embodiments, for Formula (V), A is bicyclic heterocyclyl (e.g., 2,7-dimethylimidazo[1,2-a]pyridinyl); B is monocyclic heterocyclyl (e.g., piperidinyl); L¹ and L² are absent; Y is C(R^{6a})(R^{6b}) (e.g., CH₂); each R² is hydrogen; m is 0; and n is 2. In some embodiments, the compound of Formula (V) is Compound 218, or a pharmaceutically acceptable salt, solvate, hydrate, tautomer, or stereoisomer thereof.

In some embodiments, for Formula (V), A is bicyclic heterocyclyl (e.g., 2,7-dimethyl-2H-indazolyl); B is monocyclic heterocyclyl (e.g., piperidinyl); L¹ and L² are absent; Y is C(R^{6a})(R^{6b}) (e.g., CH₂); each R² is hydrogen; m is 0; and n is 2. In some embodiments, the compound of Formula (V) is Compound 219, or a pharmaceutically acceptable salt, solvate, hydrate, tautomer, or stereoisomer thereof.

In some embodiments, for Formula (V), A is monocyclic heterocyclyl (e.g., pyrazolyl); B is monocyclic heterocyclyl (e.g., piperidinyl); L¹ and L² are absent; Y is C(R^{6a})(R^{6b}) (e.g., CH₂); each R² is hydrogen; m is 0; and n is 2. In some embodiments, the compound of Formula (V) is Compound 220, or a pharmaceutically acceptable salt, solvate, hydrate, tautomer, or stereoisomer thereof.

In some embodiments, for Formula (V), A is bicyclic heterocyclyl (e.g., 6,8-dimethylimidazo[1,2-a]pyrazyl); B is monocyclic heterocyclyl (e.g., piperidinyl); L¹ and L² are absent; Y is C(R^{6a})(R^{6b}) (e.g., CH₂); each R² is hydrogen; m is 0; and n is 2. In some embodiments, the compound of Formula (V) is Compound 221, or a pharmaceutically acceptable salt, solvate, hydrate, tautomer, or stereoisomer thereof.

In some embodiments, for Formula (V), A is bicyclic heterocyclyl (e.g., 6,8-dimethyl-[1,2,4]triazolo[1,5-a]pyrazyl); B is monocyclic heterocyclyl (e.g., piperidinyl); L¹ and L² are absent; Y is C(R^{6a})(R^{6b}) (e.g., CH₂); each R² is hydrogen; m is 0; and n is 2. In some embodiments, the compound of Formula (V) is Compound 222, or a pharmaceutically acceptable salt, solvate, hydrate, tautomer, or stereoisomer thereof.

In some embodiments, for Formula (V), A is bicyclic heterocyclyl (e.g., 4,6-dimethylpyrazolo[1,5-a]pyrazyl); B is monocyclic heterocyclyl (e.g., piperidinyl); L¹ and L² are absent; Y is C(R^{6a})(R^{6b}) (e.g., CH₂); each R² is hydrogen; m is 0; and n is 2. In some embodiments, the compound of Formula (V) is Compound 223, or a pharmaceutically acceptable salt, solvate, hydrate, tautomer, or stereoisomer thereof.

In some embodiments, for Formula (V), A is bicyclic heterocyclyl (e.g., 2-methylimidazo[1,2-a]pyrazyl); B is monocyclic heterocyclyl (e.g., piperidinyl); L¹ and L² are absent; Y is C(R^{6a})(R^{6b}) (e.g., CH₂); each R² is hydrogen; m is 0; and n is 2. In some embodiments, the compound of Formula (V) is Compound 224, or a pharmaceutically acceptable salt, solvate, hydrate, tautomer, or stereoisomer thereof.

In some embodiments, for Formula (V), A is bicyclic heterocyclyl (e.g., 7-fluoro-2-methyl-2H-indazolyl); B is monocyclic heterocyclyl (e.g., piperidinyl); L¹ and L² are absent; Y is C(R^{6a})(R^{6b}) (e.g., CH₂); each R² is hydrogen; m is 0; and n is 2. In some embodiments, the compound of Formula (V) is Compound 225, or a pharmaceutically acceptable salt, solvate, hydrate, tautomer, or stereoisomer thereof.

In some embodiments, for Formula (V), A is bicyclic heterocyclyl (e.g., 4-fluoro-2-methylbenzo[d]thiazolyl); B is monocyclic heterocyclyl (e.g., piperidinyl); L¹ and L² are absent; Y is C(R^{6a})(R^{6b}) (e.g., CH₂); each R² is hydrogen; m is 0; and n is 2. In some embodiments, the compound of Formula (V) is Compound 226, or a pharmaceutically acceptable salt, solvate, hydrate, tautomer, or stereoisomer thereof.

In some embodiments, for Formula (V), A is bicyclic heterocyclyl (e.g., 8-fluoro-2-methylimidazo[1,2-a]pyridinyl); B is monocyclic heterocyclyl (e.g., piperidinyl); L¹ and L² are absent; Y is C(R^{6a})(R^{6b}) (e.g., CH₂); each R² is hydrogen; m is 0; and n is 2. In some embodiments, the compound of Formula (V) is Compound 247, or a pharmaceutically acceptable salt, solvate, hydrate, tautomer, or stereoisomer thereof.

In some embodiments, for Formula (V), A is bicyclic heterocyclyl (e.g., 8-fluoro-2-methylimidazo[1,2-a]pyridinyl); B is monocyclic heterocyclyl (e.g., piperidinyl); L¹ and L² are absent; Y is N; R² is hydrogen; m is 0; and n is 1. In some embodiments, the compound of Formula (V) is Compound 248, or a pharmaceutically acceptable salt, solvate, hydrate, tautomer, or stereoisomer thereof.

In some embodiments, for Formula (V), A is bicyclic heterocyclyl (e.g., 7-fluoro-2-methyl-2H-indazolyl); B is monocyclic heterocyclyl (e.g., piperidinyl); L¹ and L² are absent; Y is N; R² is hydrogen; m is 0; and n is 1. In some embodiments, the compound of Formula (V) is Compound 249, or a pharmaceutically acceptable salt, solvate, hydrate, tautomer, or stereoisomer thereof.

In some embodiments, for Formula (V), A is bicyclic heterocyclyl (e.g., 4-fluoro-2-methylbenzo[d]oxazolyl); B is monocyclic heterocyclyl (e.g., piperidinyl); L¹ and L² are absent; Y is N; R² is hydrogen; m is 0; and n is 1. In some embodiments, the compound of Formula (V) is Compound 250, or a pharmaceutically acceptable salt, solvate, hydrate, tautomer, or stereoisomer thereof.

In some embodiments, for Formula (V), A is bicyclic heterocyclyl (e.g., 2,7-dimethyl-2H-indazolyl); B is monocyclic heterocyclyl (e.g., piperidinyl); L¹ and L² are absent; Y is N; R² is hydrogen; m is 0; and n is 1. In some embodiments, the compound of Formula (V) is Compound 251, or a pharmaceutically acceptable salt, solvate, hydrate, tautomer, or stereoisomer thereof.

In some embodiments, for Formula (V), A is bicyclic heterocyclyl (e.g., 2-methyl-8-(trifluoromethyl)imidazo[1,2-a]pyridinyl); B is monocyclic heterocyclyl (e.g., piperidinyl); L¹ and L² are absent; Y is N; R² is hydrogen; m is 0; and n is 1. In some embodiments, the compound of Formula (V) is Compound 252, or a pharmaceutically acceptable salt, solvate, hydrate, tautomer, or stereoisomer thereof.

In some embodiments, for Formula (V), A is bicyclic heterocyclyl (e.g., 4-fluoro-2-methylbenzo[d]thiazolyl); B is monocyclic heterocyclyl (e.g., piperidinyl); L¹ and L² are absent; Y is N; R² is hydrogen; m is 0; and n is 1. In some embodiments, the compound of Formula (V) is Compound 253, or a pharmaceutically acceptable salt, solvate, hydrate, tautomer, or stereoisomer thereof.

In some embodiments, for Formula (V), A is bicyclic heterocyclyl (e.g., 2,7-dimethylimidazo[1,2-a]pyridinyl); B is monocyclic heterocyclyl (e.g., piperidinyl); L¹ and L² are absent; Y is N; R² is hydrogen; m is 0; and n is 1. In some embodiments, the compound of Formula (V) is Compound 254, or a pharmaceutically acceptable salt, solvate, hydrate, tautomer, or stereoisomer thereof.

In some embodiments, for Formula (V), A is bicyclic heterocyclyl (e.g., 2-methylimidazo[1,2-a]pyrazyl); B is monocyclic heterocyclyl (e.g., piperidinyl); L¹ and L² are absent; Y is N; R² is hydrogen; m is 0; and n is 1. In some embodiments, the compound of Formula (V) is Compound 255, or a pharmaceutically acceptable salt, solvate, hydrate, tautomer, or stereoisomer thereof.

In some embodiments, for Formula (V), A is bicyclic heterocyclyl (e.g., 8-chloro-2-methylimidazo[1,2-a]pyridinyl); B is monocyclic heterocyclyl (e.g., piperidinyl); L¹ and L² are absent; Y is N; R² is hydrogen; m is 0; and n is 1. In some embodiments, the compound of Formula (V) is Compound 256, or a pharmaceutically acceptable salt, solvate, hydrate, tautomer, or stereoisomer thereof.

In some embodiments, for Formula (V), A is bicyclic heterocyclyl (e.g., 2,8-dimethylimidazo[1,2-a]pyridinyl); B is monocyclic heterocyclyl (e.g., piperidinyl); L¹ and L² are absent; Y is N; R² is hydrogen; m is 0; and n is 1. In some embodiments, the compound of Formula (V) is Compound 257, or a pharmaceutically acceptable salt, solvate, hydrate, tautomer, or stereoisomer thereof.

In some embodiments, for Formula (V), A is bicyclic heterocyclyl (e.g., 6,8-dimethylimidazo[1,2-a]pyrazyl); B is monocyclic heterocyclyl (e.g., piperidinyl); L¹ and L² are absent; Y is N; R² is hydrogen; m is 0; and n is 1. In some embodiments, the compound of Formula (V) is Compound 258, or a pharmaceutically acceptable salt, solvate, hydrate, tautomer, or stereoisomer thereof.

In some embodiments, for Formula (V), A is bicyclic heterocyclyl (e.g., 6,8-dimethyl-[1,2,4]triazolo[1,5-a]pyrazyl); B is monocyclic heterocyclyl (e.g., piperidinyl); L¹ and L² are absent; Y is N; R² is hydrogen; m is 0; and n is 1. In some embodiments, the compound of Formula (V) is Compound 259, or a pharmaceutically acceptable salt, solvate, hydrate, tautomer, or stereoisomer thereof.

In some embodiments, for Formula (V), A is bicyclic heterocyclyl (e.g., 4,6-dimethylpyrazolo[1,5-a]pyrazyl); B is monocyclic heterocyclyl (e.g., piperidinyl); L¹ and L² are absent; Y is N; R² is hydrogen; m is 0; and n is 1. In some embodiments, the compound of Formula (V) is Compound 260, or a pharmaceutically acceptable salt, solvate, hydrate, tautomer, or stereoisomer thereof.

In some embodiments, for Formula (V), A is monocyclic heterocyclyl (e.g., pyrazyl); B is monocyclic heterocyclyl (e.g., piperidinyl); L¹ and L² are absent; Y is N; R² is hydrogen; m is 0; and n is 1. In some embodiments, the compound of Formula (V) is Compound 261, or a pharmaceutically acceptable salt, solvate, hydrate, tautomer, or stereoisomer thereof.

In some embodiments, for Formula (V), A is bicyclic heterocyclyl (e.g., 2-methyl-8-(trifluoromethyl)imidazo[1,2-a]pyridinyl); B is monocyclic heterocyclyl (e.g., piperidinyl); L¹ and L² are absent; Y is N; R² is hydrogen; m is 0; and n is 1. In some embodiments, the compound of Formula (V) is Compound 262, or a pharmaceutically acceptable salt, solvate, hydrate, tautomer, or stereoisomer thereof.

In some embodiments, for Formula (V), A is bicyclic heterocyclyl (e.g., 2-methyl-2H-indazolyl); B is monocyclic heterocyclyl (e.g., piperidinyl); L¹ and L² are absent; Y is N; R² is hydrogen; m is 0; and n is 1. In some embodiments, the compound of Formula (V) is Compound 263, or a pharmaceutically acceptable salt, solvate, hydrate, tautomer, or stereoisomer thereof.

In some embodiments, for Formula (V), A is bicyclic heterocyclyl (e.g., 2,8-dimethylimidazo[1,2-b]pyridazyl); B is monocyclic heterocyclyl (e.g., pyrrolidinyl); L¹ and L² are absent; Y is N; R² is hydrogen; m is 0; and n is 1. In some embodiments, the compound of Formula (V) is Compound 264, or a pharmaceutically acceptable salt, solvate, hydrate, tautomer, or stereoisomer thereof.

In some embodiments, for Formula (V), A is bicyclic heterocyclyl (e.g., 2,8-dimethylimidazo[1,2-b]pyridazyl); B is monocyclic heterocyclyl (e.g., N-methyl piperidinyl); L¹ and L² are absent; Y is N; R² is hydrogen; m is 0; and n is 1. In some embodiments, the compound of Formula (V) is Compound 265, or a pharmaceutically acceptable salt, solvate, hydrate, tautomer, or stereoisomer thereof.

In some embodiments, for Formula (V), A is bicyclic heterocyclyl (e.g., 2,8-dimethylimidazo[1,2-b]pyridazyl); B is monocyclic heterocyclyl (e.g., N-ethyl piperidinyl); L¹ and L² are absent; Y is N; R² is hydrogen; m is 0; and n is 1. In some embodiments, the compound of Formula (V) is Compound 266, or a pharmaceutically acceptable salt, solvate, hydrate, tautomer, or stereoisomer thereof.

In some embodiments, for Formula (V), A is bicyclic heterocyclyl (e.g., 2,8-dimethylimidazo[1,2-b]pyridazyl); B is monocyclic heterocyclyl (e.g., 2,2-dimethylpiperidinyl); L¹ and L² are absent; Y is C(R^{6a})(R^{6b}) (e.g., CH₂); each R² is hydrogen; m is 0; and n is 2. In some embodiments, the compound of Formula (V) is Compound 267, or a pharmaceutically acceptable salt, solvate, hydrate, tautomer, or stereoisomer thereof.

In some embodiments, for Formula (V), A is bicyclic heterocyclyl (e.g., 2,8-dimethylimidazo[1,2-b]pyridazyl); B is monocyclic heterocyclyl (e.g., N-methyl piperidinyl); L¹ and L² are absent; Y is C(R^{6a})(R^{6b}) (e.g., CH₂); each R² is hydrogen; m is 0; and n is 2. In some embodiments, the compound of Formula (V) is Compound 268, or a pharmaceutically acceptable salt, solvate, hydrate, tautomer, or stereoisomer thereof.

In some embodiments, for Formula (V), A is bicyclic heterocyclyl (e.g., 2,8-dimethylimidazo[1,2-b]pyridazyl); B is monocyclic heterocyclyl (e.g., pyrrolidinyl); L¹ and L² are absent; Y is C(R^{6a})(R^{6b}) (e.g., CH₂); each R² is hydrogen; m is 0; and n is 2. In some embodiments, the compound of Formula (V) is Compound 269, or a pharmaceutically acceptable salt, solvate, hydrate, tautomer, or stereoisomer thereof.

In some embodiments, for Formula (V), A is bicyclic heterocyclyl (e.g., 2,8-dimethylimidazo[1,2-b]pyridazyl); B is monocyclic heterocyclyl (e.g., N-ethyl piperidinyl); L¹ and L² are absent; Y is C(R^{6a})(R^{6b}) (e.g., CH₂); each R² is hydrogen; m is 0; and n is 2. In some embodiments, the compound of Formula (V) is Compound 270, or a pharmaceutically acceptable salt, solvate, hydrate, tautomer, or stereoisomer thereof.

In some embodiments, for Formula (V), A is bicyclic heterocyclyl (e.g., 2,8-dimethylimidazo[1,2-b]pyridazyl); B is monocyclic heterocyclyl (e.g., N-methyl pyrrolidinyl); L¹ and L² are absent; Y is N; R² is hydrogen; m is 0; and n is 1. In some embodiments, the compound of Formula (V) is Compound 271, or a pharmaceutically acceptable salt, solvate, hydrate, tautomer, or stereoisomer thereof.

In some embodiments, for Formula (V), A is bicyclic heterocyclyl (e.g., 2,8-dimethylimidazo[1,2-b]pyridazyl); B is monocyclic heterocyclyl (e.g., 2-methylpiperidine); L¹ and L² are absent; Y is N; R² is hydrogen; m is 0; and n is 1. In some embodiments, the compound of Formula (V) is Compound 272, 273, 324, 328, 329, or a pharmaceutically acceptable salt, solvate, hydrate, tautomer, or stereoisomer thereof.

In some embodiments, for Formula (V), A is bicyclic heterocyclyl (e.g., 2,8-dimethylimidazo[1,2-b]pyridazyl); B is monocyclic heterocyclyl (e.g., 4-azaspiro[2.5]octanyl); L¹ and L² are absent; Y is N; R² is hydrogen; m is 0; and n is 1. In some embodiments, the compound of Formula (V) is Compound 274, or a pharmaceutically acceptable salt, solvate, hydrate, tautomer, or stereoisomer thereof.

In some embodiments, for Formula (V), A is bicyclic heterocyclyl (e.g., 2,8-dimethylimidazo[1,2-b]pyridazyl); B is monocyclic heterocyclyl (e.g., tetrahydro-2H-pyranyl); L¹ and L² are absent; Y is N; R² is hydrogen; m is 0; and n is 1. In some embodiments, the compound of Formula (V) is Compound 275, or a pharmaceutically acceptable salt, solvate, hydrate, tautomer, or stereoisomer thereof.

In some embodiments, for Formula (V), A is bicyclic heterocyclyl (e.g., 8-fluoro-2-methylimidazo[1,2-a]pyridinyl); B is monocyclic heterocyclyl (e.g., piperidinyl); L¹ and L² are absent; Y is C(R⁶a) (e.g., CH); R² is hydrogen; m is 0; and n is 1. In some embodiments, the compound of Formula (V) is Compound 276, or a pharmaceutically acceptable salt, solvate, hydrate, tautomer, or stereoisomer thereof.

In some embodiments, for Formula (V), A is bicyclic heterocyclyl (e.g., 2,8-dimethylimidazo[1,2-b]pyridazyl); B is monocyclic heterocyclyl (e.g., N-methyl 2-methylpiperidine); L¹ and L² are absent; Y is N; R² is hydrogen; m is 0; and n is 1. In some embodiments, the compound of Formula (V) is Compound 277, 278, 325, 330, 331, or a pharmaceutically acceptable salt, solvate, hydrate, tautomer, or stereoisomer thereof.

In some embodiments, for Formula (V), A is bicyclic heterocyclyl (e.g., 2,8-dimethylimidazo[1,2-b]pyridazyl); B is monocyclic heterocyclyl (e.g., 2,2-dimethylpiperidinyl); L¹ and L² are absent; Y is N; R² is hydrogen; m is 0; and n is 1. In some embodiments, the compound of Formula (V) is Compound 279, or a pharmaceutically acceptable salt, solvate, hydrate, tautomer, or stereoisomer thereof.

In some embodiments, for Formula (V), A is bicyclic heterocyclyl (e.g., 2,8-dimethylimidazo[1,2-b]pyridazyl); B is monocyclic heterocyclyl (e.g., 8-azabicyclo[3.2.1]octanyl); L¹ and L² are absent; Y is N; R² is hydrogen; m is 0; and n is 1. In some embodiments, the compound of Formula (V) is Compound 280, 326, 332, 333, 334 or a pharmaceutically acceptable salt, solvate, hydrate, tautomer, or stereoisomer thereof.

In some embodiments, for Formula (V), A is bicyclic heterocyclyl (e.g., 2,8-dimethylimidazo[1,2-b]pyridazyl); B is monocyclic heterocyclyl (e.g., 2-methylpiperidinyl); L¹ and L² are absent; Y is C(R^{6a})(R^{6b}) (e.g., CH₂); each R² is hydrogen; m is 0; and n is 2. In some embodiments, the compound of Formula (V) is Compound 281, 327, or a pharmaceutically acceptable salt, solvate, hydrate, tautomer, or stereoisomer thereof.

In some embodiments, for Formula (V), A is bicyclic heterocyclyl (e.g., 2,8-dimethylimidazo[1,2-b]pyridazyl); B is monocyclic heterocyclyl (e.g., N-methyl pyrrolidinyl); L¹ and L² are absent; Y is C(R^{6a})(R^{6b}) (e.g., CH₂); each R² is hydrogen; m is 0; and n is 2. In some embodiments, the compound of Formula (V) is Compound 282, or a pharmaceutically acceptable salt, solvate, hydrate, tautomer, or stereoisomer thereof.

In some embodiments, for Formula (V), A is bicyclic heterocyclyl (e.g., 2,8-dimethylimidazo[1,2-b]pyridazyl); B is monocyclic heterocyclyl (e.g., azepanyl); L¹ and L² are absent; Y is N; R² is hydrogen; m is 0; and n is 1. In some embodiments, the compound of Formula (V) is Compound 283, or a pharmaceutically acceptable salt, solvate, hydrate, tautomer, or stereoisomer thereof.

In some embodiments, for Formula (V), A is bicyclic heterocyclyl (e.g., 2,8-dimethylimidazo[1,2-b]pyridazyl); B is monocyclic heterocyclyl (e.g., 2-ethylpiperidinyl); L¹ and L² are absent; Y is N; R² is hydrogen; m is 0; and n is 1. In some embodiments, the compound of Formula (V) is Compound 304, 305, 328, 335, 336, or a pharmaceutically acceptable salt, solvate, hydrate, tautomer, or stereoisomer thereof.

In some embodiments, for Formula (V), A is bicyclic heterocyclyl (e.g., 2,8-dimethylimidazo[1,2-b]pyridazyl); B is monocyclic heterocyclyl (e.g., piperidinyl); L¹ and L² are absent; Y is C(R⁶a) (e.g., CH); R² is hydrogen; m is 0; and n is 1. In some embodiments, the compound of Formula (V) is Compound 309, or a pharmaceutically acceptable salt, solvate, hydrate, tautomer, or stereoisomer thereof.

In some embodiments, for Formula (V), A is bicyclic heterocyclyl (e.g., 2,8-dimethylimidazo[1,2-b]pyridazyl); B is monocyclic heterocyclyl (e.g., N-methyl piperidinyl); L¹ and L² are absent; Y is C(R⁶a) (e.g., CH); R² is hydrogen; m is 0; and n is 1. In some embodiments, the compound of Formula (V) is Compound 310, or a pharmaceutically acceptable salt, solvate, hydrate, tautomer, or stereoisomer thereof.

In some embodiments, for Formula (V), A is bicyclic heterocyclyl (e.g., 2,8-dimethylimidazo[1,2-b]pyridazyl); B is monocyclic heterocyclyl (e.g., 4-azaspiro[2.5]octanyl); L¹ and L² are absent; Y is N; R² is hydrogen; R³ is halo (e.g., F); m is 1; and n is 1. In some embodiments, the compound of Formula (V) is Compound 312, or a pharmaceutically acceptable salt, solvate, hydrate, tautomer, or stereoisomer thereof.

### Pharmaceutical Compositions, Kits, and Administration

The present invention provides pharmaceutical compositions comprising a compound of Formula (I), (III), or (V), e.g., a compound of Formula (I), (III), or (V) or a pharmaceutically acceptable salt, solvate, hydrate, tautomer, or stereoisomer, as described herein, and optionally a pharmaceutically acceptable excipient. In certain embodiments, the pharmaceutical composition described herein comprises a compound of Formula (I), (III), or (V) or a pharmaceutically acceptable salt thereof, and optionally a pharmaceutically acceptable excipient. In certain embodiments, the compound of Formula (I), (III), or (V) or a pharmaceutically acceptable salt, solvate, hydrate, tautomer, or stereoisomer thereof, is provided in an effective amount in the pharmaceutical composition. In certain embodiments, the effective amount is a therapeutically effective amount. In certain embodiments, the effective amount is a prophylactically effective amount.

Pharmaceutical compositions described herein can be prepared by any method known in the art of pharmacology. In general, such preparatory methods include the steps of bringing the compound of Formula (I), (III), or (V) (the “active ingredient”) into association with a carrier and/or one or more other accessory ingredients, and then, if necessary and/or desirable, shaping and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition of the invention will vary, depending upon the identity, size, and/or condition of the subject treated and further depending upon the route by which the composition is to be administered. By way of example, the composition may comprise between 0.1% and 100% (w/w) active ingredient.

The term “pharmaceutically acceptable excipient” refers to a non-toxic carrier, adjuvant, diluent, or vehicle that does not destroy the pharmacological activity of the compound with which it is formulated. Pharmaceutically acceptable excipients useful in the manufacture of the pharmaceutical compositions of the invention are any of those that are well known in the art of pharmaceutical formulation and include inert diluents, dispersing and/or granulating agents, surface active agents and/or emulsifiers, disintegrating agents, binding agents, preservatives, buffering agents, lubricating agents, and/or oils. Pharmaceutically acceptable excipients useful in the manufacture of the pharmaceutical compositions of the invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Compositions of the present invention may be administered orally, parenterally (including subcutaneous, intramuscular, intravenous and intradermal), by inhalation spray, topically, rectally, nasally, buccally, vaginally or via an implanted reservoir. In some embodiments, provided compounds or compositions are administrable intravenously and/or orally.

The term “parenteral” as used herein includes subcutaneous, intravenous, intramuscular, intraocular, intravitreal, intra-articular, intra-synovial, intrasternal, intrathecal, intrahepatic, intraperitoneal intralesional and intracranial injection or infusion techniques. Preferably, the compositions are administered orally, subcutaneously, intraperitoneally, or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added. In some embodiments, a provided oral formulation is formulated for immediate release or sustained/delayed release. In some embodiments, the composition is suitable for buccal or sublingual administration, including tablets, lozenges and pastilles. A provided compound can also be in micro-encapsulated form.

Alternatively, pharmaceutically acceptable compositions of this invention may be administered in the form of suppositories for rectal administration. Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

For ophthalmic use, provided pharmaceutically acceptable compositions may be formulated as micronized suspensions or in an ointment such as petrolatum.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds provided herein are typically formulated in dosage unit form, e.g., single unit dosage form, for ease of administration and uniformity of dosage. It will be understood, however, that the total daily usage of the compositions of the present invention will be decided by the attending physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex and diet of the subject; the time of administration, route of administration, and rate of excretion of the specific active ingredient employed; the duration of the treatment; drugs used in combination or coincidental with the specific active ingredient employed; and like factors well known in the medical arts.

The exact amount of a compound required to achieve an effective amount will vary from subject to subject, depending, for example, on species, age, and general condition of a subject, severity of the side effects or disorder, identity of the particular compound(s), mode of administration, and the like. The desired dosage can be delivered three times a day, two times a day, once a day, every other day, every third day, every week, every two weeks, every three weeks, or every four weeks. In certain embodiments, the desired dosage can be delivered using multiple administrations (e.g., two, three, four, five, six, seven, eight, nine, ten, eleven, twelve, thirteen, fourteen, or more administrations).

In certain embodiments, an effective amount of a compound for administration one or more times a day to a 70 kg adult human may comprise about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

In certain embodiments, the compounds of Formula (I), (III), or (V) may be at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic effect.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

It will be also appreciated that a compound or composition, as described herein, can be administered in combination with one or more additional pharmaceutical agents. The compounds or compositions can be administered in combination with additional pharmaceutical agents that improve their bioavailability, reduce and/or modify their metabolism, inhibit their excretion, and/or modify their distribution within the body. It will also be appreciated that the therapy employed may achieve a desired effect for the same disorder, and/or it may achieve different effects.

The compound or composition can be administered concurrently with, prior to, or subsequent to, one or more additional pharmaceutical agents, which may be useful as, e.g., combination therapies. Pharmaceutical agents include therapeutically active agents. Pharmaceutical agents also include prophylactically active agents. Each additional pharmaceutical agent may be administered at a dose and/or on a time schedule determined for that pharmaceutical agent. The additional pharmaceutical agents may also be administered together with each other and/or with the compound or composition described herein in a single dose or administered separately in different doses. The particular combination to employ in a regimen will take into account compatibility of the inventive compound with the additional pharmaceutical agents and/or the desired therapeutic and/or prophylactic effect to be achieved. In general, it is expected that the additional pharmaceutical agents utilized in combination be utilized at levels that do not exceed the levels at which they are utilized individually. In some embodiments, the levels utilized in combination will be lower than those utilized individually.

Exemplary additional pharmaceutical agents include, but are not limited to, anti-proliferative agents, anti-cancer agents, anti-diabetic agents, anti-inflammatory agents, immunosuppressant agents, and a pain-relieving agent Pharmaceutical agents include small organic molecules such as drug compounds (e.g., compounds approved by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells.

Also encompassed by the invention are kits (e.g., pharmaceutical packs). The inventive kits may be useful for preventing and/or treating a proliferative disease or a non-proliferative disease, e.g., as described herein. The kits provided may comprise an inventive pharmaceutical composition or compound and a container (e.g., a vial, ampule, bottle, syringe, and/or dispenser package, or other suitable container). In some embodiments, provided kits may optionally further include a second container comprising a pharmaceutical excipient for dilution or suspension of an inventive pharmaceutical composition or compound. In some embodiments, the inventive pharmaceutical composition or compound provided in the container and the second container are combined to form one-unit dosage form.

Thus, in one aspect, provided are kits including a first container comprising a compound described herein, or a pharmaceutically acceptable salt, solvate, hydrate, tautomer, or stereoisomer thereof, or a pharmaceutical composition thereof. In certain embodiments, the kit of the disclosure includes a first container comprising a compound described herein, or a pharmaceutically acceptable salt thereof, or a pharmaceutical composition thereof. In certain embodiments, the kits are useful in preventing and/or treating a disease, disorder, or condition described herein in a subject (e.g., a proliferative disease or a non-proliferative disease). In certain embodiments, the kits further include instructions for administering the compound, or a pharmaceutically acceptable salt, solvate, hydrate, tautomer, or stereoisomer thereof, or a pharmaceutical composition thereof, to a subject to prevent and/or treat a proliferative disease or a non-proliferative disease.

### Methods of Use

Described herein are compounds useful for modulating splicing. In some embodiments, a compound of Formula (I), (III), or (V) may be used to alter the amount, structure, or composition of a nucleic acid (e.g., a precursor RNA, e.g., a pre-mRNA, or the resulting mRNA) by increasing or decreasing splicing at a splice site. In some embodiments, increasing or decreasing splicing results in modulating the level or structure of a gene product (e.g., an RNA or protein) produced. In some embodiments, a compound of Formula (I), (III), or (V) may modulate a component of the splicing machinery, e.g., by modulating the interaction with a component of the splicing machinery with another entity (e.g., nucleic acid, protein, or a combination thereof). The splicing machinery as referred to herein comprises one or more spliceosome components. Spliceosome components may comprise, for example, one or more of major spliceosome members (U1, U2, U4, U5, U6 snRNPs), or minor spliceosome members (U11, U12, U4atac, U6atac snRNPs) and their accessory splicing factors.

In another aspect, the present disclosure features a method of modifying of a target (e.g., a precursor RNA, e.g., a pre-mRNA) through inclusion of a splice site in the target, wherein the method comprises providing a compound of Formula (I), (III), or (V). In some embodiments, inclusion of a splice site in a target (e.g., a precursor RNA, e.g., a pre-mRNA, or the resulting mRNA) results in addition or deletion of one or more nucleic acids to the target (e.g., a new exon, e.g. a skipped exon). Addition or deletion of one or more nucleic acids to the target may result in an increase in the levels of a gene product (e.g., RNA, e.g., mRNA, or protein).

In another aspect, the present disclosure features a method of modifying a target (e.g., a precursor RNA, e.g., a pre-mRNA, or the resulting mRNA) through exclusion of a splice site in the target, wherein the method comprises providing a compound of Formula (I), (III), or (V). In some embodiments, exclusion of a splice site in a target (e.g., a precursor RNA, e.g., a pre-mRNA) results in deletion or addition of one or more nucleic acids from the target (e.g., a skipped exon, e.g. a new exon). Deletion or addition of one or more nucleic acids from the target may result in a decrease in the levels of a gene product (e.g., RNA, e.g., mRNA, or protein). In other embodiments, the methods of modifying a target (e.g., a precursor RNA, e.g., a pre-mRNA, or the resulting mRNA) comprise suppression of splicing at a splice site or enhancement of splicing at a splice site (e.g., by more than about 0.5%, e.g., 1%, 5%, 10%, 15%, 20%, 25%, 30%, 35%, 40%, 45%, 50%, 55%, 60%, 65%, 70%, 75%, 80%, 85%, 90%, 95%, 99%, or more), e.g., as compared to a reference (e.g., the absence of a compound of Formula (I), (III), or (V), or in a healthy or diseased cell or tissue).

The methods described herein can be used to modulate splicing, e.g., of a nucleic acid comprising a particular sequence (e.g., a target sequence). Exemplary genes encoding a target sequence (e.g., a target sequence comprising DNA or RNA, e.g., pre-mRNA) include, inter alia, ABCA4, ABCA9, ABCB1, ABCB5, ABCC9, ABCD1, ACADL, ACADM, ACADSB, ACSS2, ACTB, ACTG2, ADA, ADAL, ADAM10, ADAM15, ADAM22, ADAM32, ADAMTS12, ADAMTS13, ADAMTS20, ADAMTS6, ADAMTS9, ADAR, ADCY3, ADCY 10, ADCY8, ADNP, ADRBK2, AFP, AGL, AGT, AHCTF1, AHR, AKAP10, AKAP3, AKNA, ALAS1, ALS2CL, ALB, ALDH3A2, ALG6, AMBRA1, ANK3, ANTXR2, ANXA10, ANXA11, ANGPTL3, AP2A2, AP4E1, APC, APOA1, APOB, APOC3, APOH, AR, ARID2, ARID3A, ARID3B, ARFGEF1, ARFGEF2, ARHGAP1, ARHGAP8, ARHGAP18, ARHGAP26, ARHGEF18, ARHGEF2, ARPC3, ARS2, ASH1L, ASH1L-IT1, ASNSD1, ASPM, ATAD5, ATF1, ATG4A, ATG16L2, ATM, ATN1, ATP11C, ATP6V1G3, ATP13A5, ATP7A, ATP7B, ATR, ATXN2, ATXN3, ATXN7, ATXN10, AXIN1, B2M, B4GALNT3, BBS4, BCL2, BCL2L1, BCL2-like 11 (BIM), BCL11B, BBOX1, BCS1L, BEAN1, BHLHE40, BMPR2, BMP2K, BPTF, BRAF, BRCA1, BRCA2, BRCC3, BRSK1, BRSK2, BTAF1, BTK, C2orf55, C4orf29, C6orf118, C9orf43, C9orf72, C10orf137, C11orf30, C11orf65, C11orf70, C11orf87, C12orf51, C13orf1, C13orf15, C14orf101, C14orf118, C15orf29, C15orf42, C15orf60, C16orf33, C16orf38, C16orf48, C18orf8, C19orf42, C1orf107, C1orf114, C1orf130, C1orf149, C1orf27, C1orf71, C1orf94, C1R, C20orf74, C21orf70, C3orf23, C4orf18, C5orf34, C8B, C8orf33, C9orf114, C9orf86, C9orf98, C3, CA11, CAB39, CACHD1, CACNA1A, CACNA1B, CACNA1C, CACNA2D1, CACNA1G, CACNA1H, CALCA, CALCOCO2, CAMK1D, CAMKK1, CAPN3, CAPN9, CAPSL, CARD11, CARKD, CASZ1, CAT, CBLB, CBX1, CBX3, CCDC102B, CCDC11, CCDC15, CCDC18, CCDC5, CCDC81, CCDC131, CCDC146, CD4, CD274, CD1B, CDC14A, CDC16, CDC2L5, CDC42BPB, CDCA8, CDH10, CDH11, CDH24, CDH8, CDH9, CDK5RAP2, CDK6, CDK8, CDK11B, CD33, CD46, CDH1, CDH23, CDK6, CDK11B, CDK13, CEBPZ, CEL, CELSR3, CENPA, CENPI, CENPT, CENTB2, CENTG2, CEP110, CEP170, CEP192, CETP, CFB, CFTR, CFH, CGN, CGNL1, CHAF1A, CHD9, CHIC2, CHL1, CHN1, CHM, CLEC16A, CL1C2, CLCN1, CLINT1, CLK1, CLPB, CLPTM1, CMIP, CMYA5, CNGA3, CNOT1, CNOT7, CNTN6, COG3, COL11A1, COL11A2, COL12A1, COL14A1, COL15A1, COL17A1, COL19A1, COL1A1, COL1A2, COL2A1, COL3A1, COL4A1, COL4A2, COL4A5, COL4A6, COL5A2, COL6A1, COL7A1, COL9A1, COL9A2, COL22A1, COL24A1, COL25A1, COL29A1, COLQ, COMTD1, COPA, COPB2, COPS7B, COPZ2, CPSF2, CPXM2, CR1, CRBN, CRYZ, CREBBP, CRKRS, CSE1L, CSTB, CSTF3, CT45-6, CTNNB1, CUBN, CUL4B, CUL5, CXorf41, CXXC1, CYBB, CYFIP2, CYP3A4, CYP3A43, CYP3A5, CYP4F2, CYP4F3, CYP17, CYP19, CYP24A1, CYP27A1, DAB1, DAZ2, DCBLD1, DCC, DCTN3, DCUN1D4, DDA1, DDEF1, DDX1, DDX24, DDX4, DENND2D, DEPDC2, DES, DGAT2, DHFR, DHRS7, DHRS9, DHX8, DIP2A, DMD, DMTF1, DNAH3, DNAH8, DNAI1, DNAJA4, DNAJC13, DNAJC7, DNMT1, DNTTIP2, DOCK4, DOCK5, DOCK10, DOCK11, DOT1L, DPP3, DPP4, DPY19L2P2, DR1, DSCC1, DVL3, DUX4, DYNC1H1, DYSF, E2F1, E2F3, E2F8, E4F1, EBF1, EBF3, ECM2, EDEM3, EFCAB3, EFCAB4B, EFNA4, EFTUD2, EGFR, EIF3A, ELA1, ELA2A, ELF2, ELF3, ELF4, EMCN, EMD, EML5, ENO3, ENPP3, EP300, EPAS1, EPB41L5, EPHA3, EPHA4, EPHB1, EPHB2, EPHB3, EPS15, ERBB4, ERCC1, ERCC8, ERGIC3, ERMN, ERMP1, ERN1, ERN2, ESR1, ESRRG, ETS2, ETV3, ETV4, ETV5, ETV6, EVC2, EWSR1, EXO1, EXOC4, F3, F11, F13A1, F5, F7, F8, FAH, FAM13A1, FAM13B1, FAM13C1, FAM134A, FAM161A, FAM176B, FAM184A, FAM19A1, FAM20A, FAM23B, FAM65C, FANCA, FANCC, FANCG, FANCM, FANK1, FAR2, FBN1, FBXO15, FBXO18, FBXO38, FCGBP, FECH, FEZ2, FGA, FGD6, FGFR2, FGFR1OP, FGFR1OP2, FGFR2, FGG, FGR, FIX, FKBP3, FLI1, FLJ35848, FLJ36070, FLNA, FN1, FNBP1L, FOLH1, FOSL1, FOSL2, FOXK1, FOXM1, FOXO1, FOXP4, FRAS1, FUT9, FXN, FZD3, FZD6, GAB1, GABPA, GALC, GALNT3, GAPDH, GART, GAS2L3, GATA3, GATAD2A, GBA, GBGT1, GCG, GCGR, GCK, GFI1, GFM1, GHI, GHR, GHV, GJA1, GLA, GLT8D1, GNA11, GNAQ, GNAS, GNB5, GOLGB1, GOLT1A, GOLT1B, GPATCH1, GPR158, GPR160, GPX4, GRAMD3, GRHL1, GRHL2, GRHPR, GRIA1, GRIA3, GRIA4, GRIN2B, GRM3, GRM4, GRN, GSDMB, GSTCD, GSTO2, GTF2I, GTPBP4, HADHA, HAND2, HBA2, HBB, HCK, HDAC3, HDAC5, HDX, HEPACAM2, HERC1, HES7, HEXA, HEXB, HHEX, HIPK3, HLA-DPB1, HLA-G, HLCS, HLTF, HMBS, HMGA1, HMGCL, HNF1A, HNF1B, HNF4A, HNF4G, HNRNPHJ, HOXC10, HP1BP3, HPGD, HPRT1, HPRT2, HSF1, HSF4, HSF2BP, HSPA9, HSPG2, HTT, HXA, ICA1, IDH1, IDS, IFI44L, IKBKAP, IKZF1, IKZF3, IL1R2, IL5RA, IL7RA, MUT, INPP5D, INSR, INTS3, INTU, IP04, IP08, IQGAP2, IRF2, IRF4, IRF8, IRX3, ISL1, ISL2, ITFG1, ITGA6, ITGAL, ITGB1, ITGB2, 1TGB3, ITGB4, ITIH1, ITPR2, IWS1, JAK1, JAK2, JAG1, JMJD1C, JPH3, KALRN, KAT6A, KATNAL2, KCNN2, KCNT2, KDM2A, KIAA0256, KIAA0528, KIAA0564, KIAA0586, KIAA1033, KIAA1166, KIAA1219, KIAA1409, KIAA1622, KIAA1787, KIF3B, KIF15, KIF16B, KIF5A, KIF5B, KIF9, KIN, KIR2DL5B, KIR3DL2, KIR3DL3, KIT, KLF3, KLF5, KLF7, KLF10, KLF12, KLF16, KLHL20, KLK12, KLKB1, KMT2A, KMT2B, KPNA5, KRAS, KREMEN1, KRIT1, KRT5, KRTCAP2, KYNU, L1CAM L3MBTL, L3MBTL2, LACE1, LAMA1, LAMA2, LAMA3, LAMB1, LARP7, LDLR, LEF1, LENG1, LGALS3, LGMN, LHCGR, LHX3, LHX6, LIMCH1, LIMK2, LIN28B, LIN54, LMBRD1, LMBRD2, LMLN, LMNA, LMO2, LMO7, LOC389634, LOC390110, LPA, LPCAT2, LPL, LRP4, LRPPRC, LRRK2, LRRC19, LRRC42, LRWD1, LUM, LVRN, LYN, LYST, MADD, MAGI1, MAGT1, MALT1, MAP2K1, MAP4K4, MAPK8IP3, MAPK9, MAPT, MARC1, MARCH5, MATN2, MBD3, MCF2L2, MCM6, MDGA2, MDM4, ASXL1, FUS, SPR54, MECOM, MEF2C, MEF2D, MEGF10, MEGF11, MEMO1, MET, MGA, MGAM, MGAT4A, MGAT5, MGC16169, MGC34774, MKKS, MIB1, MIER2, MITF, MKL2, MLANA, MLH1, MLL5, MLX, WE, MPDZ, MPI, MRAP2, MRPL11, MRPL39, MRPS28, MRPS35, MS4A13, MSH2, MSH3, MSMB, MST1R, MTDH, MTERF3, MTF1, MTF2, MTIF2, MTHFR, MUC2, MUT, MVK, MYB, MYBL2, MYC, MYCBP2, MYH2, MYRF, MYT1, MY019, MY03A, MY09B, MYOM2, MYOM3, NAG, NARG1, NARG2, NCOA1, NDC80, NDFIP2, NEB, NEDD4, NEK1, NEK5, NEK11, NF1, NF2, NFATC2, NFE2L2, NFIA, NFIB, NFIX, NFKB1, NFKB2, NFKBIL2, NFRKB, NFYA, NFYB, NIPA2, NKAIN2, NKAP, NLRC3, NLRC5, NLRP3, NLRP7, NLRP8, NLRP13, NME1, NME1-NME2, NME2, NME7, NOL10, NOP561, NOS1, NOS2A, NOTCH1, NPAS4, NPM1, NR1D1, NR1H3, NR1H4, NR4A3, NR5A1, NRXN1, NSMAF, NSMCE2, NT5C, NT5C2, NT5C3, NUBP1, NUBPL, NUDT5, NUMA1, NUP88, NUP98, NUP160, NUPL1, OAT, OAZ1, OBFC2A, OBFC2B, OLIG2, OMA1, OPA1, OPN4, OPTN, OSBPL11, OSBPL8, OSGEPL1, OTC, OTX2, OVOL2, OXT, PA2G4, PADI4, PAH, PAN2, PAOX, PAPOLG, PARD3, PARP1, PARVB, PAWR, PAX3, PAX8, PBGD, PBRM1, PBX2, PCBP4, PCCA, PCGF2, PCNX, PCOTH, PDCD4, PDE4D, PDE8B, PDE10A, PD1A3, PDH1, PDLIM5, PDXK, PDZRN3, PELI2, PDK4, PDS5A, PDS5B, PGK1, PGM2, PHACTR4, PHEX, PHKB, PHLDB2, PHOX2B, PHTF1, PIAS1, PIEZO1, PIGF, PIGN, PIGT, PIK3C2G, PIK3CA, PIK3CD, PIK3CG, PIK3RI, PIP5K1A, PITRM1, PIWIL3, PKD1, PKHD1L1, PKD2, PKIB, PKLR, PKM1, PKM2, PLAGL2, PLCB1, PLCB4, PLCG1, PLD1, PLEKHA5, PLEKHA7, PLEKHM1, PLKR, PLXNC1, PMFBP1, POLN, POLR3D, POMT2, POSTN, POU2AF1, POU2F2, POU2F3, PPARA, PPFIA2, PPP1R12A, PPP3CB, PPP4C, PPP4R1L, PPP4R2, PRAME, PRC1, PRDM1, PREX1, PREX2, PRIM1, PRIM2, PRKAR1A, PRKCA, PRKG1, PRMT7, PROC, PROCR, PROSC, PRODH, PROX1, PRPF40B, PRPF4B, PRRG2, PRUNE2, PSD3, PSEN1, PSMAL, PTCH1, PTEN, PTK2, PTK2B, PTPN2, PTPN3, PTPN4, PTPN11, PTPN22, PTPRD, PTPRK, PTPRM, PTPRN2, PTPRT, PUS10, PVRL2, PYGM, QRSL1, RAB11FIP2, RAB23, RAF1, RALBP1, RALGDS, RB1CC1, RBL2, RBM39, RBM45, RBPJ, RBSN, REC8, RELB, RFC4, RFT1, RFTN1, RHOA, RHPN2, RIF1, RIT1, RLN3, RMND5B, RNF11, RNF32, RNFT1, RNGTT, ROCK1, ROCK2, RORA, RP1, RP6KA3, RP11-265F1, RP13-36C9, RPAP3, RPN1, RPGR, RPL22, RPL22L1, RPS6KA6, RREB1, RRM1, RRP1B, RSK2, RTEL1, RTF1, RUFY1, RUNX1, RUNX2, RXRA, RYR3, SAAL1, SAE1, SALL4, SAT1, SATB2, SBCAD, SCN1A, SCN2A, SCN3A, SCN4A, SCN5A, SCN8A, SCNA, SCN11A, SCO1, SCYL3, SDC1, SDK1, SDK2, SEC24A, SEC24D, SEC31A, SEL1L, SENP3, SENP6, SENP7, SERPINA1, SETD3, SETD4, SETDB1, SEZ6, SFRS12, SGCE, SGOL2, SGPL1, SH2D1A, SH3BGRL2, SH3PXD2A, SH3PXD2B, SH3RF2, SH3TC2, SHOC2, SIPA1L2, SIPA1L3, SIVA1, SKAP1, SKIV2L2, SLC6A11, SLC6A13, SLC6A6, SLC7A2, SLC12A3, SLC13A1, SLC22A17, SLC25A14, SLC28A3, SLC33A1, SLC35F6, SLC38A1, SLC38A4, SLC39A10, SLC4A2, SLC6A8, SMARCA1, SMARCA2, SMARCA5, SMARCC2, SMC5, SMN2, SMOX, SMS, SMTN, SNCAIP, SNORD86, SNRK, SNRP70, SNX5, SNX6, SOD1, SOD10, SOS, SOS2, SOX5, SOX6, SOX8, SP1, SP2, SP3, SP110, SPAG9, SPATA13, SPATA4, SPATS1, SPECC1L, SPDEF, SPI1, SPINK5, SPP2, SPTA1, SRF, SRM, SRP72, SSX3, SSX5, SSX9, STAG1, STAG2, STAMBPLI, STARD6, STAT1, STAT3, STAT5A, STAT5B, STATE, STK17B, STX3, STXBP1, SUCLG2, SULF2, SUPT6H, SUPT16H, SV2C, SYCP2, SYT6, SYCPI, SYTL3, SYTL5, TAF2, TARDBP, TBC1D3G, TBC1D8B, TBC1D26, TBC1D29, TBCEL, TBK1, TBP, TBPL1, TBR1, TBX, TCEB3, TCF3, TCF4, TCF7L2, TCFL5, TCF12, TCP11L2, TDRD3, TEAD1, TEAD3, TEAD4, TECTB, TEK, TERF1, TERF2, TET2, TFAP2A, TFAP2B, TFAP2C, TFAP4, TFDP1, TFRC, TG, TGM7, TGS1, THAP7, THAP12, THOC2, TIAL1, TIAM2, TIMM50, TLK2, TM4SF20, TM6SF1, TMEM27, TMEM77, TMEM56, TMEM94A, TMF1, TMPRSS6, TNFRSF10A, TNFRSF10B, TNFRSF8, TNK2, TNKS, TNKS2, TOM1L1, TOM1L2, TOP2B, TP53, TP53INP1, TP53BP2, TP53I3, TP63, TRAF3IP3, TRAPPC2, TRIM44, TRIM65, TRIML1, TRIML2, TRPM3, TRPM5, TRPM7, TRPS1, TSC1, TSC2, TSHB, TSPAN7, TTC17, TTF1, TTLL5, TTLL9, TTN, TTPAL, TTR, TUSC3, TXNDC10, UBE3A, UCK1, UGT1A1, UHRF1BP1, UNC45B, UNC5C, USH2A, USF2, USPJ, USP6, USP18, USP38, USP39, UTP20, UTP15, UTP18, UTRN, UTX, UTY, UVRAG, UXT, VAPA, VEGFA, VPS29, VPS35, VPS39, VT11A, VT11B, VWA3B, WDFY2, WDR16, WDR17, WDR26, WDR44, WDR67, WDTC1, WRN, WRNIP1, WT1, WWC3, XBP1, XRN1, XRN2, XX-FW88277, YAP1, YARS, YBX1, YGM, YY1, ZBTB18, ZBTB20, ZC3HAV1, ZC3HC1, ZC3H7A, ZDHHC19, ZEB1, ZEB2, ZFPM1, ZFYVE1, ZFX, ZIC2, ZNF37A, ZNF91, ZNF114, ZNF155, ZNF169, ZNF205, ZNF236, ZNF317, ZNF320, ZNF326, ZNF335, ZNF365, ZNF367, ZNF407, ZNF468, ZNF506, ZNF511, ZNF511-PRAP1, ZNF519, ZNF521, ZNF592, ZNF618, ZNF763, and ZWINT.

Additional exemplary genes encoding a target sequence (e.g., a target sequence comprising DNA or RNA, e.g., pre-mRNA) include genes include A1ICF, A4GALT, AAR2, ABAT, ABCA11P, ZNF721, ABCA5, ABHD10, ABHD13, ABHD2, ABHD6, AC000120.3, KRIT1, AC004076.1, ZNF772, AC004076.9, ZNF772, AC004223.3, RAD51D, AC004381.6, AC006486.1, ERF, AC007390.5, AC007780.1, PRKAR1A, AC007998.2, INO80C, AC009070.1, CMC2, AC009879.2, AC009879.3, ADHFE1, AC010487.3, ZNF816-ZNF321P, ZNF816, AC010328.3, AC010522.1, ZNF587B, AC010547.4, ZNF19, AC012313.3, ZNF497, AC012651.1, CAPN3, AC013489.1, DET1, AC016747.4, C2orf74, ACO20907.6, FXYD3, AC021087.5, PDCD6, AHRR, AC022137.3, ZNF761, AC025283.3, NAA60, AC027644.4, RABGEF1, AC055811.2, FLCN, AC069368.3, ANKDD1A, AC073610.3, ARF3, AC074091.1,GPN1, AC079447.1, LIPT1, AC092587.1, AC079594.2, TRIM59, AC091060.1,C18orf21, AC092143.3, MC1R, AC093227.2, ZNF607, AC093512.2, ALDOA, AC098588.1, ANAPC10, AC107871.1, CALML4, AC114490.2, ZMYM6, AC138649.1, NIPA1, AC138894.1, CLN3, AC139768.1, AC242426.2, CHD1L, ACADM, ACAP3, ACKR2,RP11-141M3.5, KRBOX1, ACMSD, ACOT9, ACPS, ACPL2, ACSBG1, ACSF2, ACSF3, ACSL1, ACSL3, ACVR1, ADAL, ADAM29, ADAMTS10, ADAMTSL5, ADARB1, ADAT2, ADCK3, ADD3, ADGRG1, ADGRG2, ADH1B, ADIPOR1, ADNP, ADPRH, AGBL5, AGPAT1, AGPAT3, AGR2, AGTR1, AHDC1, AHI1, AHNAK, AIFM1, AIFM3, AIMP2, AK4, AKAP1, AKNAD1, CLCC1, AKR1A1, AKT1, AKT1S1, AKT2, AL139011.2, PEX19, AL157935.2, ST6GALNAC6, AL358113.1,TJP2, AL441992.2, KYAT1, AL449266.1,CLCC1, AL590556.3, LINC00339, CDC42, ALAS1, ALB, ALDH16A1, ALDH1B1, ALDH3A1, ALDH3B2, ALDOA, ALKBH2, ALPL, AMD1, AMICA1, AMN1, AMOTL2, AMY1B, AMY2B, ANAPC10, ANAPC11, ANAPC15, ANG, RNASE4, AL163636.2, ANGEL2, ANGPTL1, ANKMY1, ANKRD11, ANKRD28, ANKRD46, ANKRD9, ANKS3, ANKS3,RP11-127120.7, ANKS6, ANKZF1, ANPEP, ANXA11, ANXA2, ANXA8L2, AL603965.1, AOC3, AP000304.12, CRYZL1, AP000311.1, CRYZL1, AP000893.2,RAB30, AP001267.5, ATP5MG, AP002495.2, AP003175.1, OR2AT4, AP003419.1, CLCF1, AP005263.1, ANKRD12, AP006621.5, AP006621.1, AP1G1, AP3M1, AP3M2, APBA2, APBB1, APLP2, APOA2, APOL1, APOL3, APTX, ARAP1, STARD10, ARF4, ARFIP1, ARFIP2, ARFRP1, ARHGAP11A, ARHGAP33, ARHGAP4, ARHGEF10, ARHGEF3, ARHGEF35, OR2A1-AS1, ARHGEF35, OR2A1-AS1, ARHGEF34P, ARID1B, ARHGEF35, OR2A20P, OR2A1-AS1, ARHGEF9, ARL1, ARL13B, ARL16, ARL6, ARMC6, ARMC8, ARMCX2, ARMCX5, RP4-769N13.6, ARMCX5-GPRASP2, BHLHB9, ARMCX5-GPRASP2,GPRASP1, ARMCX5-GPRASP2,GPRASP2, ARMCX6, ARNT2, ARPP19, ARRB2, ARSA, ARTS, ASB3,GPR75-ASB3, ASCC2, ASNS, ASNS, AC079781.5, ASPSCR1, ASS1, ASUN, ATE1, ATF1, ATF7IP2, ATG13, ATG4D, ATG7, ATG9A, ATM, ATOX1, ATP1B3, ATP2C1, ATP5F1A, ATPSG2, ATPSJ, ATP5MD, ATP5PF, ATP6AP2, ATP6V0B, ATP6V1C1, ATP6V1D, ATP7B, ATXN1, ATXN1L,IST1, ATXN3, ATXN7L1, AURKA, AURKB, AXDND1, B3GALNT1, B3GALT5, AF064860.1, B3GALT5,AF064860.5, B3GNT5, B4GALT3, B4GALT4, B9D1, BACH1, BAIAP2, BANF1, BANF2, BAX, BAZ2A, BBIP1, BCHE, BCL2L14, BCL6, BCL9L, BCS1L, BDH1, BDKRB2,AL355102.2, BEST1, BEST3, BEX4, BHLHB9, BID, BIN3, BIRC2, BIVM, BIVM-ERCC5, BIVM, BLCAP, BLK, BLOC1S1, RP11-644F5.10, BLOC1S6, AC090527.2, BLOC1S6, RP11-96020.4, BLVRA, BMF, BOLA1, BORCS8-MEF2B, BORCS8, BRCA1, BRD1, BRDT, BRINP3, BROX, BTBD10, BTBD3, BTBD9, BTD, BTF3L4, BTNL9, BUB1B-PAK6, PAK6, BUB3, C10orf68, C11orf1, C11orf48, C11orf54, C11orf54,AP001273.2, C11orf57, C11orf63, C11orf82, C12orf23, C12orf4, C12orf65, C12orf79, C14orf159, C14orf93, C17orf62, C18orf21, C19orf12, C19orf40, C19orf47, C19orf48, C19orf54, CID, C1GALT1, C1QB, C1QTNF1, C1S, C1orf101, C1orf112, C1orf116, C1orf159, C1orf63, C2, C2,CFB, C20orf27, C21orf58, C2CD4D, C2orf15, LIPT1, MRPL30, C2orf80, C2orf81, C3orf14, C3orf1 7, C3orf18, C3orf22, C3orf33,AC104472.3, C4orf33, C5orf28, C5orf34, C6orf118, C6orf203, C6orf211, C6orf48, C7orf50, C7orf55, C7orf55-LUC7L2, LUC7L2, C8orf44-SGK3,C8orf44, C8orf59, C9,DAB2, C9orf153, C9orf9, CA5BPI,CA5B, CABYR, CALCA, CALCOCO1, CALCOCO2, CALM1, CALM3, CALML4, RP11-315D16.2, CALN1, CALU, CANT1, CANX, CAP1, CAPN12, CAPS2, CARD8, CARHSP1, CARNS1, CASC1, CASP3, CASP7, CBFA2T2, CBS, CBY1, CCBL1, CCBL2, RBMXL1, CCDC12, CCDC126, CCDC14, CCDC149, CCDCI50, CCDC169-SOHLH2, CCDC169, CCDC171, CCDC37, CCDC41, CCDC57, CCDC63, CCDC7, CCDC74B, CCDC77, CCDC82, CCDC90B, CCDC91, CCDC92, CCNE1, CCHCR1, CCL28, CCNB1IP1, CCNC, CCND3, CCNG1, CCP110, CCR9, CCT7, CCT8, CD151, CD1D, CD200, CD22, CD226, CD276, CD36, CD59, CDC26, CDC42, CDC42SE1, CDC42SE2, CDHR3, CDK10, CDK16, CDK4, CDKAL1, CDKL3,CTD-2410N18.4, CDKN1A, CDKN2A, CDNF, CEBPZOS, CELF1, CEMIP, CENPK, CEP170B, CEP250, CEP57, CEP57L1, CEP63, CERS4, CFL1, CFL2, CFLAR, CGNL1, CHCHD7, CHD1L, CHD8, CHFR,ZNF605, CHIA, CHID1, CHL1, CHM, CHMP1A, CHMP3, RNF103—CHMP3, CHRNA2, CIDEC, CIRBP, CITED1, CKLF-CMTM1, CMTM1, CKMT1B, CLDN12,CTB-13L3.1, CLDND1,AC021660.3, CLDND1,CPOX, CLHC1, CLIP1, CLUL1, CMC4, MTCP1, CNDP2, CNFN, CNOT1, CNOT6, CNOT7, CNOT8, CNR1, CNR2, CNTFR, CNTRL, COA1, COASY, COCH, COL8A1, COLCA1, COLEC11, COMMD3-BM11, BMI1, COPS5, COPS7B, COQ8A, CORO6, COTL1, COX14,RP4-60503.4, COX7A2, COX7A2L, COX7B2, CPA4, CPA5, CPEB1, CPNE1, AL109827.1, RBM12, CPNE1, RP1-309K20.6, RBM12, CPNE3, CPSF3L, CPT1C, CREB3L2, CREM, CRP, CRYZ, CS,AC073896.1, CS, RP11-977G19.10, CSAD, CSDE1, CSF2RA, CSGALNACT1, CSK, CSNK2A1, CSRNP2, CT45A4, CT45A4,CT45A5, CT45A6, CTBP2, CTCFL, CTD-2116N17.1, KIAA0101, CTD-2349B8.1, SYT17, CTD-2528L19.4, ZNF607, CTD-2619J13.8, ZNF497, CTNNA1, CTNNBIP1, CTNND1, CTPS2, CTSB, CTSL, CTTN, CUL2, CUL9, CWC15, CXorf40B, CYB561A3, CYBC1, CYLD, CYP11A1, CYP2R1, CYP4B1, CYP4F22, DAG1, DAGLB,KDELR2, DARS, DBNL, DCAF11, DCAF8,PEX19, DCLRE1C, DCTD, DCTN1, DCTN4, DCUN1D2, DDR1, DDX11, DDX19B, AC012184.2, DDX19B, RP11-529K1.3, DDX25, DDX39B, ATP6V1G2-DDX39B, SNORD84, DDX42, DDX60L, DEDD, DEDD2, DEFA1, DEFA1B, DEFA1B, DEFA3, DENND1C, DENND2A, DENND4B, DET1, DGKA, DGKZ, DGLUCY, DHRS4L2, DHRS9, DHX40, DIABLO, AC048338.1, DIAPH1, DICER1, DKKL1, DLG1, DLG3, DLST, DMC1, DMKN, DMTF1, DMTN, DNAJC14, DNAJC19, DNAL1, DNASE1L1, DNMT3A, DOC2A, DOCK8, DOK1, DOPEY1, DPAGT1, DPP8, DRAM2, DRD2, DROSHA, DSN1, DTNA, DTX2, DTX3, DUOX1, DUOXA1, DUS2, DUSP10, DUSP13, DUSP18, DUSP22, DYDC1, DYDC2, DYNLL1, DYNLT1, DYRK1A, DYRK2, DYRK4, RP11-500M8.7, DZIP1L, E2F6, ECHDC1, ECSIT, ECT2, EDC3, EDEM1, EDEM2, MMP24-AS1, RP4-614O4.11, EEF1AK1NMT, EEF1D, EFEMP1, EFHC1, EGFL7, EHF, EI24, EIF1AD, EIF2B5, EIF4G1, EIF2B5, POLR2H, EIF3E, EIF3K, EIF4E3, EIF4G1, ELF1, ELMO2, ELMOD1, AP000889.3, ELMOD3, ELOC, ELOF1, ELOVL1, ELOVL7, ELP1, ELP6, EML3, EMP3, ENC1, ENDOV, ENO1, ENPP5, ENTHD2, ENTPD6, EP400NL, EPB41L1, EPDR1, NME8, EPHX1, EPM2A, EPN1, EPN2, EPN3, EPS8L2, ERBB3, ERC1, ERCC1, ERG, ERI2, ERI2, DCUN1D3, ERLIN2, ERMARD, ERRFI1, ESR2,RP11-544I20.2, ESRRA, ESRRB, ESRRG, ETFA, ETFRF1, ETV1, ETV4, ETV7, EVA1A, EVC2, EVX1, EXD2, EXO5, EXOC1, EXOC2, FAAP24, FABP6, FADS1, FADS2, FAHD2B, FAM107B, FAM111A, FAM111B, FAM114A1, FAM114A2, FAM115C, FAM115C,FAM115D, FAM120B, FAM133B, FAM135A, FAM153A, FAM153B, FAM154B, FAM156A, FAM156B, FAM168B, FAM172A, FAM182B, FAM192A, FAM19A2, FAM200B, FAM220A, FAM220A, AC009412.1, FAM222B, FAM227B, FAM234A, AC004754.1, FAM3C, FAM45A, FAM49B, FAM60A, FAM63A, FAM81A, FAM86B1, FAM86B2, FANCI, FANK1, FAR2, FAXC, FAXDC2, FBF1, FBH1, FBXL4, FBXO18, FBXO22, FBXO31, FBXO41, FBXO44, FBXO45, FBXW9, FCHO1, FCHSD2, FDFT1, FDPS, FER, FETUB, FGD4, FGF1, FGFR1, FGFRL1, FGL1, FHL2, FIBCD1, FIGNL1, FIGNL1,DDC, FKBP5, FKRP, FLRT2, FLRT3, FMC1, LUC7L2, FMC1-LUC7L2, FNDC3B, FOLH1, FOLR1, FOXP1, FOXK1, FOXM1, FOXO1, FOXP4, AC097634.4, FOXRED1, FPR1, FPR2, FRG1B, FRS2, FTO, FTSJ1, FUK, FUT10, FUT3, FUT6, FXYD3, FZD3, G2E3, GAA, GABARAPL1, GABPB1, GABRA5, GAL3ST1, GALE, GALNT11, GALNT14, GALNT6, GAPVD1, GARNL3, GAS2L3, GAS8, GATA1, GATA2, GATA4, GBA, GCNT1, GDPD2, GDPD5, GEMIN7,MARK4, GEMIN8, GGA3, GGACT, AL356966.1, GGPS1, GHRL, GIDS, GIGYF2, GIMAP8, GIPC1, GJB1, GJB6, GLB1L, GLI1, GLT8D1, GMFG, GMPR2, GNAI2, GNAQ,GNB1, GNB2, GNE, GNG2, GNGT2, GNPDA1, GNPDA2, GOLGA3,CHFR, GOLGA4, GOLPH3L, GOLT1B, GPBP1L1, GPER1, GPR116, GPR141,EPDR1, GPR155, GPR161, GPR56, GPR63, GPR75-ASB3,ASB3, GPR85, GPSM2, GRAMD1B, GRB10, GRB7, GREM2, GRIA2, GSDMB, GSE1, GSN, GSTA4, GSTZ1, GTDC1, GTF2H1, GTF2H4, VARS2, GTF3C2, GUCY1A3, GUCY1B3, GUK1, GULP1, GYPC, GYS1, GZF1, HAGH, HAO2, HAPLN3, HAVCR1, HAX1, HBG2, AC104389.4, HBG2, AC104389.4, HBE1, HBG2, AC104389.4, HBE1,OR51B5, HBG2,HBE1, AC104389.28, HBS1L, HCFC1R1, HCK, HDAC2, HDAC6, HDAC7, HDLBP, HEATR4, HECTD4, HEXIM2, HHAT, HHATL, CCDC13, HINFP, HIRA, C22orf39, HIVEP3, HJV, HKR1, HLF, HMBOX1, HMGA1, HMGB3, HMGCR, HMGN4, HMOX2, HNRNPC, HNRNPD, HNRNPH1, HNRNPH3, HNRNPR, HOMER3, HOPX, HOXA3, HOXB3, HOXB3,HOXB4, HOXC4, HOXD3, HOXD3,HOXD4, HPCAL1, HPS4, HPS5, HRH1, HS3ST3A1, HSH2D, HSP90AA1, HSPD1, HTT, HUWE1, HYOU1, IAH1, ICA1L, ICAM2, ICE2, ICK, IDH2, IDH3G, IDS, IFI27, IFI44, IFT20, IFT22, IFT88, IGF2, INS-IGF2, IGF2BP3, IGFBP6, IKBKAP, IKBKB, IL11, IL18BP, IL18RAP, IL1RAP, IL1RL1, IL18R1, IL1RN, IL32, IL4I1,NUP62,AC011452.1, IL4I1,NUP62,CTC-326K19.6, IL6ST, ILVBL, IMMP1L, IMPDH1, INCA1, ING1, INIP, INPP1, INPP5J, INPP5K, INSIG2, INTS11, INTS12, INTS14, IP6K2, IP6K3, IPO11, LRRC70, IQCE, IQGAP3, IRAK4, IRF3, IRF5, IRF6, ISG20, IST1, ISYNA1, ITFG2, ITGB1BP1, ITGB7, IT1H4, RP5-966M1.6, ITPRIPL1, JADE1, JAK2, JARID2, JDP2, KANK1, KANK1,RP11-31F19.1, KANK2, KANSL1L, KAT6A, KBTBD2, KBTBD3, KCNAB2, KCNE3, KCNG1, KCNH16, KCNJ9, KCNMB2,AC117457.1,LINC01014, KCTD20, KCTD7,RABGEF1, KDM1B, KDM4A,AL451062.3, KHNYN, KIAA0040, KIAA0125, KIAA0196, KIAA0226L, PPP1R2P4, KIAA0391, KIAA0391, AL121594.1, KIAA0391, PSMA6, KIAA0753, KIAA0895, KIAA0895L, KIAA1191, KIAA1407, KIAA1841, C2orf74, KIF12, KIF14, KIF27, KIF9, KIFC3, KIN, KIRREL1, KITLG, KLC1, APOPT1, AL139300.1, KLC4, KLHDC4, KLHDC8A, KLHL13, KLHL18, KLHL2, KLHL24, KLHL7, KLK11, KLK2, KLKS, KLK6, KLK7, KNOP1, KRBA2, AC135178.2, KRBA2, RP11-849F2.7, KRIT1, KRT15, KRT8, KTN1, KXD1, KYAT3, RBMXL1, KYNU, L3MBTL1, LACC1, LARGE, LARP4, LARP7, LAT2, LBHD1, LCA5, LCA5L, LCTL, LEPROTL1, LGALS8, LGALS9C, LGMN, LHFPL2, LIG4, LIMCH1, LIMK2, LIMS2, LINC00921, ZNF263, LIPF, LLGL2, LMAN2L, LMCD1, LMF1, RP11-161M6.2, LMO1, LMO3, LOXHD1, LPAR1, LPAR2, LPAR4, LPAR5, LPAR6, LPHN1, LPIN2, LPIN3, LPP, LRFN5, LRIF1, LRMP, LRRC14, LRRC20, LRRC24, C8orf82, LRRC39, LRRC42, LRRC48, LRRC4C, LRRC8A, LRRC8B, LRRD1, LRTOMT, LRTOMT, AP000812.5, LSM7, LTB4R, LTBP3, LUC7L2, FMC1-LUC7L2, LUC7L3, LUZP1, LYG1, LYL1, LYPD4, LYPD6B, LYRM1, LYRM5, LYSMD4, MACC1, MAD1L1, MAD1L1, AC069288.1, MAEA, MAFF, MAFG, MAFK, MAGEA12,CSAG4, MAGEA2, MAGEA2B, MAGEA4, MAGEB1, MAGOHB, MAN2A2, MANBAL, MAOB, MAP2K3, MAP3K7CL, MAP3K8, MAP7, MAP9, MAPK6, MAPK7, MAPK8, MAPKAPJ, 10-Mar, 7-Mar, 8-Mar, MARK2, MASP1, MATK, MATR3, MATR3,SNHG4, MB, MBD5, MBNL1, MBOAT7, MCC, MCFD2, MCM9, MCOLN3, MCRS1, MDC1, MDGA2, MDH2, MDM2, ME1, MEAK7, MECR, MED4, MEF2A, MEF2B,BORCS8-MEF2B, MEF2BNB-MEF2B, MEF2B, MEF2BNB, MEF2C, MEF2D, MEGF10, MEI1, MEIS2, MELK, MET, METTL13, METTL23, MFF, MFN2, MFSD2A, MGST3, MIB2, MICAL1, MICAL3, MICOS10, NBL1,MICOS10-NBL1, MID1, MINA, MINOS1-NBL1,MINOS1, MIOS, MIPOL1, MIS12, MKLN1, MKNK1, MKNK1,MOB3C, MLF2, MLH1, MMP17, MOBP, MOCS1, MOGS, MOK, MORF4L1, MPC1, MPC2, MPG, MPI, MPP1, MPP2, MPPE1, MPST, MRAS, MRO, MROH1, MROH7-TTC4, MROH7, MRPL14, MRPL24, MRPL33,BABAM2, MRPL33, BRE, MRPL47, MRPL48, MRPL55, MRRF, MRTFA, MRTFB, MRVI1, MS4A1, MS4A15, MS4A3, MS4A6E,MS4A7,MS4A 14, MSANTD3, MSANTD4, MSH5,MSH5-SAPCD1, MSL2, MSRB3, MSS51, MTCP1, CMC4, MTERF, MTERF1, MTERF3, MTERFD2, MTERFD3, MTF2, MTG2, MTHFD2, MTHFD2L, MTIF2, MTIF3, MTMR10, MTRF1, MTRR, MTUS2, MUTYH, MVK, MX1, MX2, MYH10, MYL12A, MYB, MYD88, MYL5, MYLIP, MYNN, MYO15A, MYO1B, MYOM2, MZF1, N4BP2L2, NAA60, NAB1, NAE1, NAGK, NAP1L1, NAP1L4, NAPG, NARFL, NARG2, NAT1, NAT10, NBPF11, WI2-3658N16. 1, NBPF12, NBPF15, NBPF24, NBPF6, NBPF9, NBR1, NCAPG2, NCBP2, NCEH1, NCOA1, NCOA4, NDC1, NDRG1, NDRG2, NDRG4, NDST1, NDUFAF6, NDUFB2, ND UFC1, ND UFS1, NDUFS8, NDUFV1, NEDD1, NEIL1, NEIL2, NEK10, NEK11, NEK6, NEK9, NELFA, NEU4, NFAT5, NFE2, NFE2L2, AC019080.1, NFRKB, NFYA, NFYC, NIF3L1, NIPA2, NKIRAS1, NKX2-1, NLRC3, NME1,NME1-NME2,NME2, NME1-NME2, NME2, NME4, NME6, NME9, NOD1, NOL10, NOL8, NONO, NPAS1, NPIPA8, RP11-1212A22.1, NPIPB3, NPIPB4, NPIPB9, NPL, NPM1, NPPA, NQO2, NR1H3, NR2C2, NR2F2, NR4A1, NRDC, NREP, NRF1, NRG4, NRIP1, NSD2, NSDHL, NSG1, NSMCE2, NSRP1, NT5C2, NTF4, NTMT1, NTNG2, NUBP2, NUCB2, NUDT1, NUDT2, NUDT4, NUF2, NUMBL, NUP50, NUP54, NUP85, NVL, NXF1, NXPE1, NXPE3, OARD1, OAT, OAZ2, OCIAD1, OCLN, ODF2, OGDHL, OGFOD2, AC026362.1, OGFOD2, RP11-197N18.2, OLA1, OPRL1, OPTN, OR2H1, ORAI2, ORMDL1, ORMDL2, ORMDL3, OSBPL2, OSBPL3, OSBPL5, OSBPL9, OSER1, OSGIN1, OSR2, P2RX4, P2RY2, P2RY6, P4HA2, PABPC1, PACRGL, PACSIN3, PADI1, PAIP2, PAK1, PAK3, PAK4, PAK7, PALB2, PANK2, PAQR6, PARP11, PARVG, PASK, PAX6, PBRM1, PBXIP1, PCBP3, PCBP4,AC115284.1, PCBP4, RP11-155D18.14, RP11-155D18. 12, PCGF3, PCGF5, PCNP, PCSK9, PDCD10, PDCD6, AHRR, PDDC1, PDGFRB, PDIA6, PDIK1L, PDLIM7, PDP1, PDPK1, PDPN, PDZD11, PEA15, PEX2, PEX5, PEX5L, PFKM, PFN4, PGAP2, PGAP2, AC090587.2, PGAP3, PGM3, PGPEP1, PHB, PHC2, PHF20, PHF21A, PHF23, PHKB, PHLDB1, PHOSPHO1, PHOSPHO2, KLHL23, PI4KB, PIAS2, PICALM, PIF1, PIGN, PIGO, PIGT, PIK3CD, PILRB, STAG3LSP-PVRIG2P-PILRB, PIP5K1B, PIR, PISD, PIWIL4,FUT4, PKD2, PKIA, PKIG, PKM, PKN2, PLA1A, PLA2G2A, PLA2G5, PLA2G7, PLAC8, PLAGL1, PLD1, PLD3, PLEKHA1, PLEKHA2, PLEKHA6, PLEKHG5, PLIN1, PLS1, PLS3, PLSCR1, PLSCR2, PLSCR4, PLXNB1, PLXNB2, PMP22, PMS1, PNISR, PNKP,AKT1S1, PNMT, PNPLA4, PNPLA8, PNPO, PNRC1, POC1B, POFUT1, POLB, POLD1, POLH, POLI, POLL, POLR1B, POM121, POM121C,AC006014.7, POM121C, AC211429.1, POMC, POMT1, POP1, PORCN, POU5F1, PSORS1C3, PPARD, PPARG, PPHLN1, PPIL3, PPIL4, PPM1A, PPM1B,AC013717.1, PPP1CB, PPP1R11, PPP1R13L, PPP1R26, PPP1R9A, PPP2R2B, PPP3CA, PPP6R1, PPP6R3, PPT2,PPT2-EGFL8, EGFL8, PPWD1, PRDM2, PRDM8, PRELID3A, PREPL, PRICKLE1, PRKAG1, PRMT2, PRMT5, PRMT7, PROM1, PRPS1, PRPSAP2, PRR14L, PRR15L, PRR5,PRR5-ARHGAP8, PRR5L, PRR7, PRRC2B, PRRT4, PRSS50, PRSS45, PRSS44, PRUNE, PRUNE1, PSEN1, PSMA2, PSMF1, PSORS1C1, PSPH, PSRC1, PTBP3, PTHLH, PTK2, PTPDC1, PTPRM, PUF60, PUM2, PUS1, PUS10, PXN, PXYLP1, PYCR1, QRICH1, R3HCC1L, R3HDM2, RAB17, RAB23, RAB3A, RAB3D,TMEM205, RAB4B-EGLN2, EGLN2, AC008537.1, RABSB, RAB7L1, RABL2A, RABL2B, RABL5, RACGAP1, RAD17, RAD51L3-RFFL, RAD51D, RAD52, RAE1, RAI14, RAI2, RALBP1, RAN, RANGAP1, RAP1A, RAP1B, RAP1GAP, RAPGEF4, RAPGEFL1, RASGRP2, RASSF1, RBCK1, RBM12B, RBM14, RBM4, RBM14-RBM4, RBM23, RBM4, RBM14-RBM4, RBM47, RBM7,AP002373. 1, RBM7, RP11-212D19.4, RBMS2, RBMY1E, RBPJ, RBPMS, RBSN, RCBTB2, RCC1, RCC1, SNHG3, RCCD1, RECQL, RELL2, REPIN1, AC073111.3, REPIN1, ZNF775, RER1, RERE, RFWD3, RFX3, RGL2, RGMB, RGS11, RGS3, RGS5, AL592435.1, RHBDD1, RHNO1, TULP3, RHOC, AL603832.3, RHOC,RP11-426L16.10, RHOH, RIC8B, RIMKLB, RIN1, RIPK2, RIT1, RLIM, RNASE4,ANG,AL163636.6, RNASEK, RNASEK-C17orf49, RNF111, RNF123, RNF13, RNF14, RNF185, RNF216, RNF24, RNF32, RNF34, RNF38, RNF4, RNF44, RNH1, RNMT, RNPS1, RO60, ROPN1, ROPN1B, ROR2, RP1-102H19.8, C6orf163, RP1-283E3.8,CDK11A, RP11-120M18.2,PRKAR1A, RP11-133K1.2, PAK6, RP11-164J13.1,CAPN3, RP11-21J18.1, ANKRD12, RP11-322E11.6,INO80C, RP11-337C18.10,CHD1L, RP11-432B6.3, TRIM59, RP11-468E2.4,IRF9, RP11-484M3.5,UPK1B, RP11-517H2.6, CCR6, RP11-613M10.9, SLC25A51, RP11-659G9.3, RAB30, RP11-691N7.6,CTNND1, RP11-849H4.2, RP11-896J10.3, NKX2-1, RP11-96O20.4,SQRDL, RP11-986E7.7, SERPINA3, RP4-769N13.6, GPRASPJ, RP4-769N13.6,GPRASP2, RP4-798P15.3, SEC16B, RP5-1021I20.4, ZNF410, RP6-109B7.3, FLJ27365, RPE, RPH3AL, RPL15, RPL17, RPL17-C18orf32,RPL17, RPL23A, RPL36,HSD11B1L, RPP38, RPS20, RPS27A, RPS3A, RPS6KA3, RPS6KC1, RPS6KL1, RPUSD1, RRAGD, RRAS2, RRBP1, RSL1D1, RSRC2, RSRP1, RUBCNL, RUNX1T1, RUVBL2, RWDD1, RWDD4, S100A13,AL162258.1, S100A13,RP1-178F15.5, S100A16, S100A4, S100A3, S100A6, S100PBP, SAA1, SACM1L, SAMD4B, SAR1A, SARAF, SARNP,RP11-76217.5, SCAMP5, SCAP, SCAPER, SCFD1, SCGB3A2, SC1N, SCML1, SCNN1D, SCO2, SCOC, SCRN1, SDC2, SDC4, SEC13, SEC14L1, SEC14L2, SEC22C, SEC23B, SEC24C, SEC61G, SEMA4A, SEMA4C, SEMA4D, SEMA6C, SENP7, SEPP1, 11-Sep, 2-Sep, SERGEF, AC055860.1, SERP1, SERPINA1, SERPINA5, SERPINB6, SERPING1, SERPINH1, SERTAD3, SETD5, SFMBT1, AC096887.1, SFTPA1, SFTPA2, SFXN2, SGCD, SGCE, SGK3, SGK3,C8orf44, SH2B1, SH2D6, SH3BP1,Z83844.3, SH3BP2, SH3BP5, SH3D19, SH3YL1, SHC1, SHISA5, SHMT1, SHMT2, SHOC2, SHROOM1, SIGLEC5,SIGLEC14, SIL1, SIN3A, SIRT2, SIRT6, SKP1, STAT4, AC104109.3, SLAIN1, SLC10A3, SLC12A9, SLC14A1, SLC16A6, SLC1A2, SLC1A6, SLC20A2, SLC25A18, SLC25A19, SLC25A22, SLC25A25, SLC25A29, SLC25A30, SLC25A32, SLC25A39, SLC25A44, SLC25A45, SLC25A53, SLC26A11, SLC26A4, SLC28A1, SLC29A1, SLC2A14, SLC2AS, SLC2A8, SLC35B2, SLC35B3, SLC35C2, SLC37A1, SLC38A1, SLC38A11, SLC39A13, SLC39A14, SLC41A3, SLC44A3, SLC4A7, SLC4A8, SLC5A10, SLC5A11, SLC6A1, SLC6A12, SLC6A9, SLC7A2, SLC7A6, SLC7A7, SLCO1A2, SLCO1C1, SLCO2B1, SLFN11, SLFN12, SLFNL1, SLMO1, SLTM, SLU7, SMAD2, SMAP2, SMARCA2, SMARCE1, AC073508.2, SMARCE1, KRT222, SMC6, SMG7, SMIM22, SMOX, SMPDL3A, SMTN, SMU1, SMUG1, SNAP2S, SNCA, SNRK, SNRPC, SNRPD1, SNRPD2, SNRPN, SNRPN,SNURF, SNUPN, SNX11, SNX16, SNX17, SOAT1, SOHLH2,CCDC169-SOHLH2,CCDC169, SORBS1, SORBS2, SOX5, SP2, SPART, SPATA20, SPATA21, SPATS2, SPATS2L, SPDYE2, SPECC1, SPECC1L,SPECC1L-ADORA2A, SPECC1L-ADORA2A, ADORA2A, SPEG, SPG20, SPG21, SPIDR, SPIN1, SPOCD1, SPOP, SPRR2A, SPRR2B, SPRR2E, SPRR2B, SPRR2F, SPRR2D, SPRR3, SPRY1, SPRY4, SPTBN2, SRC, SRGAP1, SRP68, SRSF11, SSX1, SSX2IP, ST3GAL4, ST3GAL6, ST5, ST6GALNAC6, ST7L, STAC3, STAG1, STAG2, STAMBP, STAMBPL1, STARD3NL, STAT6, STAU1, STAU2, AC022826.2, STAU2, RP11-463D19.2, STEAP2, STEAP3, STIL, STK25, STK33, STK38L, STK40, STMN1, STON1,STON1-GTF2A1L, STRAP, STRBP, STRC, AC011330.5, STRC, CATSPER2, STRC, CATSPER2, AC011330.5, STRC,STRCP1, STT3A, STX16-NPEPL1, NPEPL1, STX5, STX6, STX8, STXBP6, STYK1, SULT1A1, SULT1A2, SUMF2, SUN1, SUN2, SUN2, DNAL4, SUOX, SUPT6H, SUV39H2, SV2B, SYBU, SYNCRIP, SYNJ2, SYT1, SYTL4, TAB2, TACC1, TADA2B, TAF1C, TAF6,AC073842.2, TAF6, RP11-506M12.1, TAF9, TAGLN, TANK, TAPSAR1,PSMB9, TAPT1, TATDN1, TAZ, TBC1D1, TBC1D12, HELLS, TBC1D15, TBC1D3H,TBC1D3G, TBC1D5, TBC1D5,SATB1, TBCA, TBCEL, TBCEL, AP000646.1, TBL1XR1, TBP, TBX5, TBXAS1, TCAF1, TCEA2, TCEAL4, TCEAL8, TCEAL9, TCEANC, TCEB1, TCF19, TCF25, TCF4, TCP1, TCP10L, AP000275.65, TCP11, TCP11L2, TCTN1, TDG, TDP1, TDRD7, TEAD2, TECR, TENC1, TENT4A, TEX264, TEX30, TEX37, TFDP1, TFDP2, TFEB, TFG, TFP1,TF, TFPI, TGIF1, THAP6, THBS3, THOC5, THRAP3, THUMPD3, TIAL1, TIMM, TIMP1, TIRAP, TJAP1, TJP2, TK2, TLDC1, TLE3, TLE6, TLN1, TLR10, TM9SF1, TMBIM1, TMBIM4, TMBIM6, TMC6, TMCC1, TMCO4, TMEM126A, TMEM139, TMEM150B, Th1EM155, TMEM161B, TMEM164, TMEM168, TMEM169, TMEM175, TMEM176B, TMEM182, TMEM199,CTB-96E2.3, TMEM216, TMEM218, TMEM230, TMEM263, TMEM45A, TMEM45B, TMEM62, TMEM63B, TMEM66, TMEM68, TMEM98, TMEM9B, TMPRSS11D, TMPRSS5, TMSB15B, TMTC4, TMUB2, TMX2—CTNND1, RP11-691N7.6,CTNND1, TNFAIP2, TNFAIP8L2, SCNM1, TNFRSF10C, TNFRSFI9, TNFRSF8, TNFSF12-TNFSF13, TNFSF12, TNFSF13, TNFSF12-TNFSF13, TNFSF13, TNIP1, TNK2, TNNT1, TNRC18, TNS3, TOB2, TOM1L1, TOP1MT, TOP3B, TOX2, TP53,RP11-199F11.2, TP53I11, TP53INP2, TPCN1, TPM3P9,AC022137.3, TPT1, TRA2B, TRAF2, TRAF3, TRAPPC12, TRAPPC3, TREH, TREX1, TREX2, TRIB2, TRIM3, TRIM36, TRIM39, TRIM46, TRIM6, TRIM6-TRIM34, TRIM6-TRIM34, TRIM34, TRIM66, TRIM73, TRIT1, TRMT10B, TRMT2B, TRMT2B-AS1, TRNT1, TRO, TROVE2, TRPS1, TRPT1, TSC2, TSGA10, TSPAN14, TSPAN3, TSPAN4, TSPANS, TSPAN6, TSPAN9, TSPO, TTC12, TTC23, TTC3, TTC39A, TTC39C, TTLL1, TTLL7, TTPAL, TUBD1, TWNK, TXNL4A, TXNL4B, TXNRD1, TYK2, U2AF1, UBA2, UBA52, UBAP2, UBE2D2, UBE2D3, UBE2E3, UBE2I, UBE2J2, UBE3A, UBL7, UBXN11, UBXN7, UGDH, UGGT1, UGP2, UMAD1,AC007161.3, UNC45A, UQCC1, URGCP-MRPS24,URGCP, USMG5, USP16, USP21, USP28, USP3, USP33, USP35, USP54, USP9Y, USPL1, UTP15, VARS2, VASH2, VAV3, VDAC1, VDAC2, VDR, VEZT, VGF, VIL1, VILL, VIPR1, VPS29, VPS37C, VPS8, VPS9D1, VRK2, VWA1, VWA5A, WARS, WASF1, WASHC5, WBP5, WDHD1, WDPCP, WDR37, WDR53, WDR6, WDR72, WDR74, WDR81, WDR86, WDYHV1, WFDC3, WHSC1, WIPF1, WSCD2, WWP2, XAGE1A, XAGE1B, XKR9, XPNPEP1, XRCC3, XRN2, XXYLT1, YIF1A, YIF1B, YIPF1, YIPF5, YPEL5, YWHAB, YWHAZ, YY1AP1, ZBTB1, ZBTB14, ZBTB18, ZBTB20, ZBTB21, ZBTB25, ZBTB33, ZBTB34, ZBTB38, ZBTB43, ZBTB49, ZBTB7B, ZBTB7C, ZBTB8OS, ZC3H11A, ZBED6, ZC3H13, ZCCHC17, ZCCHC7, ZDHHC11, ZDHHC13, ZEB2, ZFAND5, ZFAND6, ZFP1, ZFP62, ZFX, ZFYVE16, ZFYVE19, ZFYVE20, ZFYVE27, ZHX2, AC016405.1, ZHX3, ZIK1, ZIM2,PEG3, ZKSCAN1, ZKSCAN3, ZKSCAN8, ZMAT3, ZMAT5, ZMIZ2, ZMYM6, ZMYND11, ZNF10,AC026786.1, ZNF133, ZNF146, ZNF16, ZNF177, ZNF18, ZNF200, ZNF202, ZNF211, ZNF219, ZNF226, ZNF227, ZNF23, AC010547.4, ZNF23, AC010547.9, ZNF239, ZNF248, ZNF25, ZNF253, ZNF254, ZNF254, AC092279.1, ZNF263, ZNF274, ZNF275, ZNF28,ZNF468, ZNF283, ZNF287, ZNF3, ZNF320, ZNF322, ZNF324B, ZNF331, ZNF334, ZNF34, ZNF350, ZNF385A, ZNF395, FBXO16, ZNF415, ZNF418, ZNF43, ZNF433-AS1, AC008770.4, ZNF438, ZNF444, ZNF445, ZNF467, ZNF480, ZNF493, ZNF493,CTD-2561J22.3, ZNF502, ZNF507, ZNF512, AC074091.1, ZNF512,RP11-158I13.2, ZNF512B, ZNF512B, SAMD10, ZNF521, ZNF532, ZNF544, AC020915.5, ZNF544, CTD-3138B18.4, ZNF559,ZNF177, ZNF562, ZNF567, ZNF569, ZNF570, ZNF571-AS1,ZNF540, ZNF577, ZNF580,ZNF581, ZNF580, ZNF581,CCDC106, ZNF600, ZNF611, ZNF613, ZNF615, ZNF619,ZNF620, ZNF639, ZNF652, ZNF665, ZNF667, ZNF668, ZNF671, ZNF682, ZNF687, ZNF691, ZNF696, ZNF701, ZNF706, ZNF707, ZNF714, ZNF717, ZNF718, ZNF720, ZNF721, ZNF730, ZNF763, ZNF780B,AC005614.5, ZNF782, ZNF786, ZNF79, ZNF791, ZNF81, ZNF83, ZNF837, ZNF839, ZNF84, ZNF845, ZNF846, ZNF865, ZNF91, ZNF92, ZNHIT3, ZSCAN21, ZSCAN25, ZSCAN30, and ZSCAN32.

In some embodiments, the gene encoding a target sequence comprises the HTT gene. In some embodiments, the gene encoding a target sequence comprises the SMN2 gene.

Exemplary genes that may be modulated by the compounds of Formula (I), (III), or (V) described herein may also include, inter alia, AC005258.1, AC005943.1, AC007849.1, AC008770.2, AC010487.3, AC011477.4, AC012651.1, AC012531.3, AC034102.2, AC073896.4, AC104472.3, AL109811.3, AL133342.1, AL137782.1, AL157871.5, AF241726.2, AL355336.1, AL358113.1, AL360181.3, AL445423.2, AL691482.3, AP001267.5, RF01169, and RF02271.

The compounds described herein may further be used to modulate a sequence comprising a particular splice site sequence, e.g., an RNA sequence (e.g., a pre-mRNA sequence). In some embodiments, the splice site sequence comprises a 5′ splice site sequence. In some embodiments, the splice site sequence comprises a 3′ splice site sequence. Exemplary gene sequences and splice site sequences (e.g., 5′ splice site sequences) include AAAgcaaguu, AAAguaaaaa, AAAguaaaau, AAAguaaagu, AAAguaaaua, AAAguaaaug, AAAguaaauu, AAAguaacac, AAAguaacca, AAAguaacuu, AAAguaagaa, AAAguaagac, AAAguaagag, AAAguaagau, AAAguaagca, AAAguaagcc, AAAguaagcu, AAAguaagga, AAAguaaggg, AAAguaaggu, AAAguaagua, AAAguaaguc, AAAguaagug, AAAguaaguu, AAAguaaucu, AAAguaauua, AAAguacaaa, AAAguaccgg, AAAguacuag, AAAguacugg, AAAguacuuc, AAAguacuug, AAAguagcuu, AAAguaggag, AAAguaggau, AAAguagggg, AAAguaggua, AAAguaguaa, AAAguauauu, AAAguauccu, AAAguaucuc, AAAguaugga, AAAguaugua, AAAguaugug, AAAguauguu, AAAguauugg, AAAguauuuu, AAAgucagau, AAAgucugag, AAAgugaaua, AAAgugagaa, AAAgugagac, AAAgugagag, AAAgugagau, AAAgugagca, AAAgugagcu, AAAgugaggg, AAAgugagua, AAAgugaguc, AAAgugagug, AAAgugaguu, AAAgugcguc, AAAgugcuga, AAAguggguc, AAAguggguu, AAAgugguaa, AAAguguaug, AAAgugugug, AAAguguguu, AAAguuaagu, AAAguuacuu, AAAguuagug, AAAguuaugu, AAAguugagu, AAAguuugua, AACguaaaac, AACguaaagc, AACguaaagg, AACguaagca, AACguaaggg, AACguaaguc, AACguaagug, AACguaaugg, AACguaguga, AACguaugua, AACguauguu, AACgugagca, AACgugagga, AACgugauuu, AACgugggau, AACgugggua, AACguguguu, AACguuggua, AAGgcaaauu, AAGgcaagag, AAGgcaagau, AAGgcaagcc, AAGgcaagga, AAGgcaaggg, AAGgcaagug, AAGgcaaguu, AAGgcacugc, AAGgcagaaa, AAGgcaggau, AAGgcaggca, AAGgcaggga, AAGgcagggg, AAGgcaggua, AAGgcaggug, AAGgcaucuc, AAGgcaugcu, AAGgcaugga, AAGgcauguu, AAGgcauuau, AAGgcgagcu, AAGgcgaguc, AAGgcgaguu, AAGgcuagcc, AAGguaaaaa, AAGguaaaac, AAGguaaaag, AAGguaaaau, AAGguaaaca, AAGguaaacc, AAGguaaacu, AAGguaaaga, AAGguaaagc, AAGguaaagg, AAGguaaagu, AAGguaaaua, AAGguaaauc, AAGguaaaug, AAGguaaauu, AAGguaacaa, AAGguaacau, AAGguaaccc, AAGguaacua, AAGguaacuc, AAGguaacug, AAGguaacuu, AAGguaagaa, AAGguaagac, AAGguaagag, AAGguaagau, AAGguaagca, AAGguaagcc, AAGguaagcg, AAGguaagcu, AAGguaagga, AAGguaaggc, AAGguaaggg, AAGguaaggu, AAGguaagua, AAGguaaguc, AAGguaagug, AAGguaaguu, AAGguaauaa, AAGguaauac, AAGguaauag, AAGguaauau, AAGguaauca, AAGguaaucc, AAGguaaucu, AAGguaauga, AAGguaaugc, AAGguaaugg, AAGguaaugu, AAGguaauua, AAGguaauuc, AAGguaauug, AAGguaauuu, AAGguacaaa, AAGguacaag, AAGguacaau, AAGguacacc, AAGguacacu, AAGguacagg, AAGguacagu, AAGguacaua, AAGguacaug, AAGguacauu, AAGguaccaa, AAGguaccag, AAGguaccca, AAGguacccu, AAGguaccuc, AAGguaccug, AAGguaccuu, AAGguacgaa, AAGguacggg, AAGguacggu, AAGguacguc, AAGguacguu, AAGguacuaa, AAGguacuau, AAGguacucu, AAGguacuga, AAGguacugc, AAGguacugu, AAGguacuuc, AAGguacuug, AAGguacuuu, AAGguagaaa, AAGguagaac, AAGguagaca, AAGguagacc, AAGguagacu, AAGguagagu, AAGguagaua, AAGguagcaa, AAGguagcag, AAGguagcca, AAGguagccu, AAGguagcua, AAGguagcug, AAGguagcuu, AAGguaggaa, AAGguaggag, AAGguaggau, AAGguaggca, AAGguaggcc, AAGguaggcu, AAGguaggga, AAGguagggc, AAGguagggg, AAGguagggu, AAGguaggua, AAGguagguc, AAGguaggug, AAGguagguu, AAGguaguaa, AAGguaguag, AAGguagucu, AAGguagugc, AAGguagugg, AAGguaguuc, AAGguaguuu, AAGguauaaa, AAGguauaau, AAGguauaca, AAGguauacu, AAGguauaua, AAGguauauc, AAGguauaug, AAGguauauu, AAGguaucac, AAGguaucag, AAGguauccc, AAGguauccu, AAGguaucuc, AAGguaucug, AAGguaucuu, AAGguaugaa, AAGguaugac, AAGguaugag, AAGguaugau, AAGguaugca, AAGguaugcc, AAGguaugcu, AAGguaugga, AAGguauggc, AAGguauggg, AAGguaugua, AAGguauguc, AAGguaugug, AAGguauguu, AAGguauuaa, AAGguauuac, AAGguauuag, AAGguauuau, AAGguauucc, AAGguauuga, AAGguauugu, AAGguauuua, AAGguauuuc, AAGguauuug, AAGguauuuu, AAGgucaaau, AAGgucaaga, AAGgucaagu, AAGgucacag, AAGgucagaa, AAGgucagac, AAGgucagag, AAGgucagca, AAGgucagcc, AAGgucagcg, AAGgucagcu, AAGgucagga, AAGgucaggc, AAGgucaggg, AAGgucaggu, AAGgucagua, AAGgucaguc, AAGgucagug, AAGgucaguu, AAGgucauag, AAGgucaucu, AAGguccaca, AAGguccaga, AAGguccaua, AAGgucccag, AAGgucccuc, AAGguccuuc, AAGgucgagg, AAGgucuaau, AAGgucuacc, AAGgucuaua, AAGgucuccu, AAGgucucug, AAGgucucuu, AAGgucugaa, AAGgucugag, AAGgucugga, AAGgucuggg, AAGgucugua, AAGgucuguu, AAGgucuucu, AAGgucuuuu, AAGgugaaac, AAGgugaaag, AAGgugaaau, AAGgugaacu, AAGgugaagc, AAGgugaagg, AAGgugaagu, AAGgugaaua, AAGgugaaug, AAGgugaauu, AAGgugacaa, AAGgugacag, AAGgugacau, AAGgugacug, AAGgugacuu, AAGgugagaa, AAGgugagac, AAGgugagag, AAGgugagau, AAGgugagca, AAGgugagcc, AAGgugagcg, AAGgugagcu, AAGgugagga, AAGgugaggc, AAGgugaggg, AAGgugaggu, AAGgugagua, AAGgugaguc, AAGgugagug, AAGgugaguu, AAGgugauaa, AAGgugauca, AAGgugaucc, AAGgugauga, AAGgugaugc, AAGgugaugu, AAGgugauua, AAGgugauug, AAGgugauuu, AAGgugcaca, AAGgugcauc, AAGgugcccu, AAGgugccug, AAGgugcgug, AAGgugcguu, AAGgugcucc, AAGgugcuga, AAGgugcugc, AAGgugcugg, AAGgugcuua, AAGgugcuuu, AAGguggaua, AAGguggcua, AAGguggcug, AAGguggcuu, AAGgugggaa, AAGgugggag, AAGgugggau, AAGgugggca, AAGgugggcc, AAGgugggcg, AAGgugggga, AAGguggggu, AAGgugggua, AAGgugggug, AAGguggguu, AAGgugguaa, AAGgugguac, AAGgugguau, AAGguggugg, AAGgugguua, AAGgugguuc, AAGgugguuu, AAGguguaag, AAGgugucaa, AAGgugucag, AAGgugucug, AAGgugugaa, AAGgugugag, AAGgugugca, AAGgugugga, AAGguguggu, AAGgugugua, AAGguguguc, AAGgugugug, AAGguguguu, AAGguguucu, AAGguguugc, AAGguguugg, AAGguguuug, AAGguuaaaa, AAGguuaaca, AAGguuaagc, AAGguuaauu, AAGguuacau, AAGguuagaa, AAGguuagau, AAGguuagca, AAGguuagcc, AAGguuagga, AAGguuaggc, AAGguuagua, AAGguuaguc, AAGguuagug, AAGguuaguu, AAGguuauag, AAGguuauga, AAGguucaaa, AAGguucaag, AAGguuccuu, AAGguucggc, AAGguucguu, AAGguucuaa, AAGguucuga, AAGguucuua, AAGguugaau, AAGguugacu, AAGguugagg, AAGguugagu, AAGguugaua, AAGguugcac, AAGguugcug, AAGguuggaa, AAGguuggca, AAGguuggga, AAGguugggg, AAGguuggua, AAGguugguc, AAGguuggug, AAGguugguu, AAGguuguaa, AAGguugucc, AAGguugugc, AAGguuguua, AAGguuuacc, AAGguuuaua, AAGguuuauu, AAGguuuccu, AAGguuucgu, AAGguuugag, AAGguuugca, AAGguuugcc, AAGguuugcu, AAGguuugga, AAGguuuggu, AAGguuugua, AAGguuuguc, AAGguuugug, AAGguuuuaa, AAGguuuuca, AAGguuuucg, AAGguuuugc, AAGguuuugu, AAGguuuuuu, AAUgcaagua, AAUgcaaguc, AAUguaaaca, AAUguaaaua, AAUguaaauc, AAUguaaaug, AAUguaaauu, AAUguaacua, AAUguaagaa, AAUguaagag, AAUguaagau, AAUguaagcc, AAUguaagcu, AAUguaagga, AAUguaagua, AAUguaaguc, AAUguaagug, AAUguaaguu, AAUguaauca, AAUguaauga, AAUguaaugu, AAUguacauc, AAUguacaug, AAUguacgau, AAUguacgua, AAUguacguc, AAUguacgug, AAUguacucu, AAUguaggca, AAUguagguu, AAUguaucua, AAUguaugaa, AAUguaugua, AAUguaugug, AAUguauguu, AAUgucagag, AAUgucagau, AAUgucagcu, AAUgucagua, AAUgucaguc, AAUgucagug, AAUgucaguu, AAUgucggua, AAUgucuguu, AAUgugagaa, AAUgugagca, AAUgugagcc, AAUgugagga, AAUgugagua, AAUgugaguc, AAUgugagug, AAUgugaguu, AAUgugauau, AAUgugcaua, AAUgugcgua, AAUgugcguc, AAUgugggac, AAUguggguc, AAUgugggug, AAUgugguuu, AAUgugugua, AAUguuaagu, AAUguuagaa, AAUguuagau, AAUguuagua, AAUguuggug, ACAgcaagua, ACAguaaaua, ACAguaaaug, ACAguaagaa, ACAguaagca, ACAguaagua, ACAguaaguc, ACAguaagug, ACAguaaguu, ACAguacgua, ACAguaggug, ACAguauaac, ACAguaugua, ACAgucaguu, ACAgugagaa, ACAgugagcc, ACAgugagcu, ACAgugagga, ACAgugaggu, ACAgugagua, ACAgugaguc, ACAgugagug, ACAgugaguu, ACAgugggua, ACAguggguu, ACAguguaaa, ACAguuaagc, ACAguuaagu, ACAguuaugu, ACAguugagu, ACAguuguga, ACCguaagua, ACCgugagaa, ACCgugagca, ACCgugaguu, ACCgugggug, ACGguaaaac, ACGguaacua, ACGguaagua, ACGguaagug, ACGguaaguu, ACGguaauua, ACGguaauuu, ACGguacaau, ACGguacagu, ACGguaccag, ACGguacggu, ACGguacgua, ACGguaggaa, ACGguaggag, ACGguaggug, ACGguaguaa, ACGguauaau, ACGguaugac, ACGguaugcg, ACGguaugua, ACGguauguc, ACGgugaaac, ACGgugaagu, ACGgugaauc, ACGgugacag, ACGgugacca, ACGgugagaa, ACGgugagau, ACGgugagcc, ACGgugagua, ACGgugagug, ACGgugaguu, ACGgugcgug, ACGguggcac, ACGguggggc, ACGgugggug, ACGguguagu, ACGgugucac, ACGgugugua, ACGguguguu, ACGguuagug, ACGguuaguu, ACGguucaau, ACUguaaaua, ACUguaagaa, ACUguaagac, ACUguaagca, ACUguaagcu, ACUguaagua, ACUguaaguc, ACUguaaguu, ACUguacguu, ACUguacugc, ACUguaggcu, ACUguaggua, ACUguauauu, ACUguaugaa, ACUguaugcu, ACUguaugug, ACUguauucc, ACUgucagcu, ACUgucagug, ACUgugaacg, ACUgugagca, ACUgugagcg, ACUgugagcu, ACUgugagua, ACUgugaguc, ACUgugagug, ACUgugaguu, ACUgugggua, ACUgugugug, ACUguuaagu, AGAgcaagua, AGAguaaaac, AGAguaaacg, AGAguaaaga, AGAguaaagu, AGAguaaauc, AGAguaaaug, AGAguaacau, AGAguaacua, AGAguaagaa, AGAguaagac, AGAguaagag, AGAguaagau, AGAguaagca, AGAguaagcu, AGAguaagga, AGAguaaggc, AGAguaaggg, AGAguaaggu, AGAguaaguc, AGAguaagug, AGAguaaguu, AGAguaauaa, AGAguaaugu, AGAguaauuc, AGAguaauuu, AGAguacacc, AGAguaccug, AGAguacgug, AGAguacucu, AGAguacuga, AGAguacuuu, AGAguagcug, AGAguaggaa, AGAguaggga, AGAguagggu, AGAguagguc, AGAguaggug, AGAguagguu, AGAguauaua, AGAguauauu, AGAguaugaa, AGAguaugac, AGAguaugau, AGAguauguc, AGAguaugug, AGAguauguu, AGAguauuaa, AGAguauuau, AGAgucagug, AGAgugagac, AGAgugagag, AGAgugagau, AGAgugagca, AGAgugagua, AGAgugaguc, AGAgugagug, AGAgugaguu, AGAgugcguc, AGAgugggga, AGAgugggug, AGAgugugug, AGAguguuuc, AGAguuagua, AGAguugaga, AGAguugagu, AGAguugguu, AGAguuugau, AGCguaagcu, AGCguaagug, AGCgugagcc, AGCgugagug, AGCguuguuc, AGGgcagagu, AGGgcagccu, AGGgcuagua, AGGguaaaga, AGGguaaaua, AGGguaaauc, AGGguaaauu, AGGguaacca, AGGguaacug, AGGguaacuu, AGGguaagaa, AGGguaagag, AGGguaagau, AGGguaagca, AGGguaagga, AGGguaaggc, AGGguaaggg, AGGguaagua, AGGguaaguc, AGGguaagug, AGGguaaguu, AGGguaauac, AGGguaauga, AGGguaauua, AGGguaauuu, AGGguacacc, AGGguacagu, AGGguacggu, AGGguaggac, AGGguaggag, AGGguaggca, AGGguaggcc, AGGguaggga, AGGguagggu, AGGguagguc, AGGguaggug, AGGguagguu, AGGguauaua, AGGguaugac, AGGguaugag, AGGguaugau, AGGguaugca, AGGguaugcu, AGGguauggg, AGGguauggu, AGGguaugua, AGGguauguc, AGGguaugug, AGGguauuac, AGGguauucu, AGGguauuuc, AGGgucagag, AGGgucagca, AGGgucagga, AGGgucaggg, AGGgucagug, AGGgucaguu, AGGguccccu, AGGgucggga, AGGgucugca, AGGgucuguu, AGGgugaaga, AGGgugacua, AGGgugagaa, AGGgugagac, AGGgugagag, AGGgugagca, AGGgugagcc, AGGgugagcu, AGGgugagga, AGGgugaggg, AGGgugaggu, AGGgugagua, AGGgugaguc, AGGgugagug, AGGgugaguu, AGGgugggga, AGGguggggu, AGGgugggua, AGGgugggug, AGGgugugua, AGGgugugug, AGGguuaaug, AGGguuagaa, AGGguuaguu, AGGguuggug, AGGguuugug, AGGguuuguu, AGUguaaaag, AGUguaaaua, AGUguaaauu, AGUguaagaa, AGUguaagag, AGUguaagau, AGUguaagca, AGUguaagcc, AGUguaagua, AGUguaagug, AGUguaaguu, AGUguaauug, AGUguaggac, AGUguagguc, AGUguaugag, AGUguaugua, AGUguauguu, AGUguauugu, AGUguauuua, AGUgucaguc, AGUgugagag, AGUgugagca, AGUgugagcc, AGUgugagcu, AGUgugagua, AGUgugaguc, AGUgugagug, AGUgugaguu, AGUgugggua, AGUgugggug, AGUgugugua, AGUguuccua, AGUguugggg, AGUguuucag, AUAguaaaua, AUAguaagac, AUAguaagau, AUAguaagca, AUAguaagua, AUAguaagug, AUAguaaguu, AUAguaggua, AUAguauguu, AUAgucucac, AUAgugagac, AUAgugagag, AUAgugagau, AUAgugagcc, AUAgugaggc, AUAgugagua, AUAgugaguc, AUAgugagug, AUAgugcguc, AUAgugugua, AUAguucagu, AUCguaagcc, AUCguaaguu, AUCguauucc, AUCgugagua, AUGgcaagcg, AUGgcaagga, AUGgcaaguu, AUGgcaggua, AUGgcaugug, AUGgcgccau, AUGgcuugug, AUGguaaaac, AUGguaaaau, AUGguaaacc, AUGguaaaga, AUGguaaaua, AUGguaaaug, AUGguaaauu, AUGguaacag, AUGguaacau, AUGguaacua, AUGguaacuc, AUGguaacuu, AUGguaagaa, AUGguaagac, AUGguaagag, AUGguaagau, AUGguaagca, AUGguaagcc, AUGguaagcu, AUGguaagga, AUGguaaggg, AUGguaagua, AUGguaaguc, AUGguaagug, AUGguaaguu, AUGguaauaa, AUGguaauau, AUGguaauga, AUGguaaugg, AUGguaauug, AUGguaauuu, AUGguacagc, AUGguacauc, AUGguaccag, AUGguaccug, AUGguacgag, AUGguacggu, AUGguagauc, AUGguagcag, AUGguagcug, AUGguaggaa, AUGguaggau, AUGguaggca, AUGguaggcu, AUGguagggg, AUGguagggu, AUGguaggua, AUGguaggug, AUGguaguuu, AUGguauagu, AUGguauaua, AUGguaucag, AUGguaucuu, AUGguaugau, AUGguaugca, AUGguaugcc, AUGguaugcg, AUGguaugcu, AUGguaugga, AUGguauggc, AUGguaugug, AUGguauguu, AUGguauuau, AUGguauuga, AUGguauuug, AUGgucaggg, AUGgucaguc, AUGgucagug, AUGgucauuu, AUGgugaaaa, AUGgugaaac, AUGgugaaau, AUGgugaacu, AUGgugaaga, AUGgugacgu, AUGgugagaa, AUGgugagac, AUGgugagag, AUGgugagca, AUGgugagcc, AUGgugagcg, AUGgugagcu, AUGgugaggc, AUGgugaggg, AUGgugagua, AUGgugaguc, AUGgugagug, AUGgugaguu, AUGgugauuu, AUGgugcgau, AUGgugcgug, AUGgugggua, AUGgugggug, AUGguggguu, AUGgugguua, AUGguguaag, AUGgugugaa, AUGgugugua, AUGgugugug, AUGguuacuc, AUGguuagca, AUGguuaguc, AUGguuagug, AUGguuaguu, AUGguucagu, AUGguucguc, AUGguuggua, AUGguugguc, AUGguugguu, AUGguuguuu, AUGguuugca, AUGguuugua, AUUgcaagua, AUUguaaaua, AUUguaagau, AUUguaagca, AUUguaagga, AUUguaaggc, AUUguaagua, AUUguaaguc, AUUguaaguu, AUUguaauua, AUUguaauuu, AUUguacaaa, AUUguaccuc, AUUguacgug, AUUguacuug, AUUguaggua, AUUguaugag, AUUguaugua, AUUgucuguu, AUUgugagcu, AUUgugagua, AUUgugaguc, AUUgugaguu, AUUgugcgug, AUUgugggug, AUUguuagug, CAAguaaaaa, CAAguaaaua, CAAguaaauc, CAAguaaaug, CAAguaaccc, CAAguaacua, CAAguaacug, CAAguaagaa, CAAguaagac, CAAguaagau, CAAguaaggu, CAAguaagua, CAAguaaguc, CAAguaagug, CAAguaaguu, CAAguaaucc, CAAguaaucu, CAAguaauua, CAAguaauuc, CAAguaauug, CAAguaauuu, CAAguacaca, CAAguacguu, CAAguacuuu, CAAguagcug, CAAguaggau, CAAguaggua, CAAguagguc, CAAguaggug, CAAguagguu, CAAguaguuu, CAAguauaac, CAAguauaug, CAAguaucuu, CAAguaugag, CAAguaugua, CAAguauguc, CAAguaugug, CAAguauguu, CAAguauuga, CAAguauuuc, CAAgucagac, CAAgucagua, CAAgucuaua, CAAgucugau, CAAgugacuu, CAAgugagaa, CAAgugagac, CAAgugagca, CAAgugaggc, CAAgugaggg, CAAgugagua, CAAgugaguc, CAAgugagug, CAAgugaucc, CAAgugaucu, CAAgugauuc, CAAgugauug, CAAgugauuu, CAAgugccuu, CAAgugggua, CAAguggguc, CAAgugggug, CAAgugugag, CAAguuaaaa, CAAguuaagu, CAAguuaauc, CAAguuagaa, CAAguuaguu, CAAguucaag, CAAguuccgu, CAAguuggua, CAAguuuagu, CAAguuucca, CAAguuuguu, CACguaagag, CACguaagca, CACguaauug, CACguaggac, CACguaucga, CACgucaguu, CACgugagcu, CACgugaguc, CACgugagug, CAGgcaagaa, CAGgcaagac, CAGgcaagag, CAGgcaagga, CAGgcaagua, CAGgcaagug, CAGgcaaguu, CAGgcacgca, CAGgcagagg, CAGgcaggug, CAGgcaucau, CAGgcaugaa, CAGgcaugag, CAGgcaugca, CAGgcaugcg, CAGgcaugug, CAGgcgagag, CAGgcgccug, CAGgcgugug, CAGguaaaaa, CAGguaaaag, CAGguaaaca, CAGguaaacc, CAGguaaaga, CAGguaaagc, CAGguaaagu, CAGguaaaua, CAGguaaauc, CAGguaaaug, CAGguaaauu, CAGguaacag, CAGguaacau, CAGguaacca, CAGguaaccg, CAGguaacgu, CAGguaacua, CAGguaacuc, CAGguaacug, CAGguaacuu, CAGguaagaa, CAGguaagac, CAGguaagag, CAGguaagau, CAGguaagcc, CAGguaagga, CAGguaaggc, CAGguaaggg, CAGguaaggu, CAGguaagua, CAGguaagug, CAGguaaguu, CAGguaauaa, CAGguaauau, CAGguaaucc, CAGguaaugc, CAGguaaugg, CAGguaaugu, CAGguaauua, CAGguaauuc, CAGguaauug, CAGguaauuu, CAGguacaaa, CAGguacaag, CAGguacaau, CAGguacaca, CAGguacacg, CAGguacaga, CAGguacagg, CAGguacagu, CAGguacaua, CAGguacaug, CAGguacauu, CAGguaccac, CAGguaccca, CAGguacccg, CAGguacccu, CAGguaccgc, CAGguaccgg, CAGguaccuc, CAGguaccug, CAGguaccuu, CAGguacgag, CAGguacgca, CAGguacgcc, CAGguacggu, CAGguacgua, CAGguacgug, CAGguacuaa, CAGguacuag, CAGguacuau, CAGguacucc, CAGguacucu, CAGguacuga, CAGguacugc, CAGguacugu, CAGguacuua, CAGguacuuu, CAGguagaaa, CAGguagaac, CAGguagaag, CAGguagaca, CAGguagacc, CAGguagaga, CAGguagauu, CAGguagcaa, CAGguagcac, CAGguagcag, CAGguagcca, CAGguagcgu, CAGguagcua, CAGguagcuc, CAGguagcug, CAGguagcuu, CAGguaggaa, CAGguaggac, CAGguaggag, CAGguaggca, CAGguaggga, CAGguagggc, CAGguagggg, CAGguagggu, CAGguaggua, CAGguagguc, CAGguaggug, CAGguagguu, CAGguaguaa, CAGguaguau, CAGguaguca, CAGguagucc, CAGguaguga, CAGguagugu, CAGguaguuc, CAGguaguug, CAGguaguuu, CAGguauaag, CAGguauaca, CAGguauaga, CAGguauauc, CAGguauaug, CAGguauauu, CAGguaucag, CAGguaucau, CAGguauccu, CAGguaucga, CAGguaucgc, CAGguaucua, CAGguaucug, CAGguaucuu, CAGguaugaa, CAGguaugac, CAGguaugag, CAGguaugau, CAGguaugca, CAGguaugcc, CAGguaugcg, CAGguaugcu, CAGguaugga, CAGguauggg, CAGguauggu, CAGguaugua, CAGguauguc, CAGguaugug, CAGguauguu, CAGguauuau, CAGguauuca, CAGguauucu, CAGguauuga, CAGguauugg, CAGguauugu, CAGguauuua, CAGguauuuc, CAGguauuug, CAGguauuuu, CAGgucaaca, CAGgucaaug, CAGgucacgu, CAGgucagaa, CAGgucagac, CAGgucagca, CAGgucagcc, CAGgucagcg, CAGgucagga, CAGgucagua, CAGgucaguc, CAGgucagug, CAGgucaguu, CAGgucaucc, CAGgucaugc, CAGgucauua, CAGgucauuu, CAGguccacc, CAGguccacu, CAGguccagu, CAGguccauc, CAGguccauu, CAGgucccag, CAGgucccug, CAGguccuga, CAGguccugc, CAGguccugg, CAGgucggcc, CAGgucggug, CAGgucguug, CAGgucucuc, CAGgucucuu, CAGgucugag, CAGgucugcc, CAGgucugcg, CAGgucugga, CAGgucuggu, CAGgucugua, CAGgucuguc, CAGgucugug, CAGgucuguu, CAGgucuucc, CAGgucuuuc, CAGgugaaag, CAGgugaaau, CAGgugaaca, CAGgugaaga, CAGgugaagg, CAGgugaaua, CAGgugaauc, CAGgugaauu, CAGgugacaa, CAGgugacau, CAGgugacca, CAGgugaccc, CAGgugaccg, CAGgugaccu, CAGgugacgg, CAGgugacua, CAGgugacuc, CAGgugacug, CAGgugagaa, CAGgugagac, CAGgugagag, CAGgugagau, CAGgugagca, CAGgugagcc, CAGgugagcg, CAGgugagcu, CAGgugagga, CAGgugaggc, CAGgugaggg, CAGgugaggu, CAGgugagua, CAGgugaguc, CAGgugagug, CAGgugaguu, CAGgugauaa, CAGgugaucc, CAGgugaucu, CAGgugaugc, CAGgugaugg, CAGgugaugu, CAGgugauua, CAGgugauuc, CAGgugauug, CAGgugauuu, CAGgugcaaa, CAGgugcaag, CAGgugcaca, CAGgugcacg, CAGgugcaga, CAGgugcagg, CAGgugcaua, CAGgugcauc, CAGgugcaug, CAGgugccaa, CAGgugccca, CAGgugcccc, CAGgugcccg, CAGgugccua, CAGgugccug, CAGgugcgaa, CAGgugcgca, CAGgugcgcc, CAGgugcgcg, CAGgugcgga, CAGgugcggu, CAGgugcgua, CAGgugcguc, CAGgugcgug, CAGgugcuag, CAGgugcuau, CAGgugcuca, CAGgugcucc, CAGgugcucg, CAGgugcugc, CAGgugcugg, CAGgugcuua, CAGgugcuuc, CAGgugcuug, CAGguggaac, CAGguggaag, CAGguggaau, CAGguggaga, CAGguggagu, CAGguggauu, CAGguggcca, CAGguggcuc, CAGguggcug, CAGgugggaa, CAGgugggac, CAGgugggag, CAGgugggau, CAGgugggca, CAGgugggcc, CAGgugggcu, CAGgugggga, CAGguggggc, CAGguggggg, CAGguggggu, CAGgugggua, CAGguggguc, CAGgugggug, CAGguggguu, CAGguggucu, CAGguggugg, CAGgugguug, CAGguguaca, CAGguguagg, CAGguguauc, CAGgugucac, CAGgugucag, CAGgugucca, CAGguguccu, CAGgugucua, CAGgugucuc, CAGgugucug, CAGgugugaa, CAGgugugac, CAGgugugag, CAGgugugau, CAGgugugca, CAGgugugcc, CAGgugugcg, CAGgugugcu, CAGgugugga, CAGguguggc, CAGgugugua, CAGguguguc, CAGgugugug, CAGguguguu, CAGguguuua, CAGguuaaaa, CAGguuaaua, CAGguuaauc, CAGguuaccu, CAGguuagaa, CAGguuagag, CAGguuagau, CAGguuagcc, CAGguuaggg, CAGguuaggu, CAGguuagua, CAGguuaguc, CAGguuagug, CAGguuaguu, CAGguuauca, CAGguuaugu, CAGguuauua, CAGguuauug, CAGguucaaa, CAGguucaac, CAGguucaag, CAGguucaca, CAGguucacg, CAGguucagg, CAGguucaug, CAGguuccag, CAGguuccca, CAGguucccg, CAGguucgaa, CAGguucgag, CAGguucuau, CAGguucugc, CAGguucuua, CAGguucuuc, CAGguucuuu, CAGguugaac, CAGguugaag, CAGguugagu, CAGguugaua, CAGguuggag, CAGguuggca, CAGguuggcc, CAGguugguc, CAGguuggug, CAGguugguu, CAGguuguaa, CAGguuguac, CAGguuguau, CAGguuguca, CAGguuguga, CAGguuguug, CAGguuuaag, CAGguuuacc, CAGguuuagc, CAGguuuagu, CAGguuucuu, CAGguuugaa, CAGguuugag, CAGguuugau, CAGguuugcc, CAGguuugcu, CAGguuuggg, CAGguuuggu, CAGguuugua, CAGguuugug, CAGguuuguu, CAGguuuucu, CAGguuuugg, CAGguuuuuc, CAGguuuuuu, CAUgcagguu, CAUguaaaac, CAUguaacua, CAUguaagaa, CAUguaagag, CAUguaagau, CAUguaagcc, CAUguaagua, CAUguaagug, CAUguaaguu, CAUguaauua, CAUguacaua, CAUguaccac, CAUguacguu, CAUguaggua, CAUguaggug, CAUguagguu, CAUguaugaa, CAUguaugua, CAUguaugug, CAUguauguu, CAUgugagaa, CAUgugagca, CAUgugagcu, CAUgugagua, CAUgugaguc, CAUgugagug, CAUgugaguu, CAUgugcgua, CAUgugggaa, CAUguggguu, CAUgugugug, CAUguguguu, CAUguuaaua, CAUguuagcc, CCAguaagau, CCAguaagca, CCAguaagcc, CCAguaagcu, CCAguaagga, CCAguaagua, CCAguaaguc, CCAguaagug, CCAguaaguu, CCAguaauug, CCAguacggg, CCAguagguc, CCAguauugu, CCAgugaggc, CCAgugagua, CCAgugagug, CCAguggguc, CCAguuaguu, CCAguugagu, CCCguaagau, CCCguauguc, CCCguauguu, CCCguccugc, CCCgugagug, CCGguaaaga, CCGguaagau, CCGguaagcc, CCGguaagga, CCGguaaggc, CCGguaaugg, CCGguacagu, CCGguacuga, CCGguauucc, CCGgucagug, CCGgugaaaa, CCGgugagaa, CCGgugaggg, CCGgugagug, CCGgugaguu, CCGgugcgcg, CCGgugggcg, CCGguugguc, CCUguaaaug, CCUguaaauu, CCUguaagaa, CCUguaagac, CCUguaagag, CCUguaagca, CCUguaagcg, CCUguaagga, CCUguaaguu, CCUguaggua, CCUguaggug, CCUguaucuu, CCUguauggu, CCUguaugug, CCUgugagaa, CCUgugagca, CCUgugaggg, CCUgugaguc, CCUgugagug, CCUgugaguu, CCUguggcuc, CCUgugggua, CCUgugugua, CCUguuagaa, CGAguaaggg, CGAguaaggu, CGAguagcug, CGAguaggug, CGAguagguu, CGAgugagca, CGCguaagag, CGGgcaggca, CGGguaagcc, CGGguaagcu, CGGguaaguu, CGGguaauuc, CGGguaauuu, CGGguacagu, CGGguacggg, CGGguaggag, CGGguaggcc, CGGguaggug, CGGguauuua, CGGgucugag, CGGgugaccg, CGGgugacuc, CGGgugagaa, CGGgugaggg, CGGgugaggu, CGGgugagua, CGGgugagug, CGGgugaguu, CGGgugauuu, CGGgugccuu, CGGgugggag, CGGgugggug, CGGguggguu, CGGguguguc, CGGgugugug, CGGguguguu, CGGguucaag, CGGguucaug, CGGguuugcu, CGUguagggu, CGUguaugca, CGUguaugua, CGUgucugua, CGUgugagug, CGUguuuucu, CUAguaaaug, CUAguaagcg, CUAguaagcu, CUAguaagua, CUAguaaguc, CUAguaagug, CUAguaaguu, CUAguaauuu, CUAguaggua, CUAguagguu, CUAguaugua, CUAguauguu, CUAgugagua, CUCguaagca, CUCguaagug, CUCguaaguu, CUCguaucug, CUCgucugug, CUCgugaaua, CUCgugagua, CUCgugauua, CUGguaaaaa, CUGguaaaau, CUGguaaacc, CUGguaaacg, CUGguaaagc, CUGguaaaua, CUGguaaauc, CUGguaaaug, CUGguaaauu, CUGguaacac, CUGguaacag, CUGguaaccc, CUGguaaccg, CUGguaacug, CUGguaacuu, CUGguaagaa, CUGguaagag, CUGguaagau, CUGguaagca, CUGguaagcc, CUGguaagcu, CUGguaagga, CUGguaaggc, CUGguaaggg, CUGguaaggu, CUGguaagua, CUGguaagug, CUGguaaguu, CUGguaauga, CUGguaaugc, CUGguaauuc, CUGguaauuu, CUGguacaac, CUGguacaau, CUGguacaga, CUGguacaua, CUGguacauu, CUGguaccau, CUGguacguu, CUGguacuaa, CUGguacuug, CUGguacuuu, CUGguagaga, CUGguagaua, CUGguagcgu, CUGguaggau, CUGguaggca, CUGguaggua, CUGguagguc, CUGguaggug, CUGguaucaa, CUGguaugau, CUGguauggc, CUGguauggu, CUGguaugua, CUGguaugug, CUGguauguu, CUGguauuga, CUGguauuuc, CUGguauuuu, CUGgucaaca, CUGgucagag, CUGgucccgc, CUGgucggua, CUGgucuggg, CUGgugaagu, CUGgugaaua, CUGgugaauu, CUGgugacua, CUGgugagaa, CUGgugagac, CUGgugagca, CUGgugagcu, CUGgugagga, CUGgugaggc, CUGgugaggg, CUGgugaggu, CUGgugagua, CUGgugaguc, CUGgugagug, CUGgugaguu, CUGgugauua, CUGgugauuu, CUGgugcaga, CUGgugcgcu, CUGgugcgug, CUGgugcuga, CUGgugggag, CUGgugggga, CUGgugggua, CUGguggguc, CUGgugggug, CUGguggguu, CUGgugugaa, CUGgugugca, CUGgugugcu, CUGguguggu, CUGgugugug, CUGguguguu, CUGguuagcu, CUGguuagug, CUGguucgug, CUGguuggcu, CUGguuguuu, CUGguuugua, CUGguuuguc, CUGguuugug, CUUguaaaug, CUUguaagcu, CUUguaagga, CUUguaaggc, CUUguaagua, CUUguaagug, CUUguaaguu, CUUguacguc, CUUguacgug, CUUguaggua, CUUguagugc, CUUguauagg, CUUgucagua, CUUgugagua, CUUgugaguc, CUUgugaguu, CUUguggguu, CUUgugugua, CUUguuagug, CUUguuugag, GAAguaaaac, GAAguaaagc, GAAguaaagu, GAAguaaaua, GAAguaaauu, GAAguaagaa, GAAguaagcc, GAAguaagcu, GAAguaagga, GAAguaagua, GAAguaagug, GAAguaaguu, GAAguaauau, GAAguaaugc, GAAguaauua, GAAguaauuu, GAAguaccau, GAAguacgua, GAAguacguc, GAAguaggca, GAAguagguc, GAAguauaaa, GAAguaugcu, GAAguaugug, GAAguauguu, GAAguauuaa, GAAgucagug, GAAgugagag, GAAgugagcg, GAAgugaggu, GAAgugaguc, GAAgugagug, GAAgugaguu, GAAgugauaa, GAAgugauuc, GAAgugcgug, GAAguguggg, GAAguguguc, GAAguuggug, GACguaaagu, GACguaagcu, GACguaagua, GACguaaugg, GACguaugcc, GACguauguu, GACgugagcc, GACgugagug, GAGgcaaaug, GAGgcaagag, GAGgcaagua, GAGgcaagug, GAGgcaaguu, GAGgcacgag, GAGgcaggga, GAGgcaugug, GAGgcgaagg, GAGguaaaaa, GAGguaaaac, GAGguaaaag, GAGguaaaau, GAGguaaacc, GAGguaaaga, GAGguaaagc, GAGguaaagu, GAGguaaaua, GAGguaaauc, GAGguaaaug, GAGguaaauu, GAGguaacaa, GAGguaacag, GAGguaacca, GAGguaaccu, GAGguaacuu, GAGguaagaa, GAGguaagag, GAGguaagau, GAGguaagca, GAGguaagcc, GAGguaagcg, GAGguaagcu, GAGguaagga, GAGguaaggc, GAGguaaggg, GAGguaaggu, GAGguaagua, GAGguaaguc, GAGguaauaa, GAGguaauac, GAGguaauau, GAGguaauca, GAGguaaucu, GAGguaaugg, GAGguaaugu, GAGguaauug, GAGguaauuu, GAGguacaaa, GAGguacaac, GAGguacaga, GAGguacagc, GAGguacagu, GAGguacaua, GAGguacauu, GAGguaccag, GAGguaccga, GAGguaccug, GAGguaccuu, GAGguacuag, GAGguacuau, GAGguacucc, GAGguacugc, GAGguacugg, GAGguacugu, GAGguacuug, GAGguacuuu, GAGguagaag, GAGguagaga, GAGguagagg, GAGguagagu, GAGguagauc, GAGguagcua, GAGguagcug, GAGguaggaa, GAGguaggag, GAGguaggca, GAGguaggcu, GAGguaggga, GAGguagggc, GAGguagggg, GAGguaggua, GAGguaggug, GAGguagguu, GAGguaguaa, GAGguaguag, GAGguaguau, GAGguagucu, GAGguagugc, GAGguagugg, GAGguaguua, GAGguaguug, GAGguauaag, GAGguauacu, GAGguauagc, GAGguauaug, GAGguauauu, GAGguaucau, GAGguaucug, GAGguaucuu, GAGguaugaa, GAGguaugac, GAGguaugag, GAGguaugcc, GAGguaugcg, GAGguaugcu, GAGguaugga, GAGguauggg, GAGguauggu, GAGguaugua, GAGguauguc, GAGguaugug, GAGguauguu, GAGguauucc, GAGguauuga, GAGguauugu, GAGguauuua, GAGguauuuc, GAGguauuug, GAGguauuuu, GAGgucaaca, GAGgucaagg, GAGgucaaug, GAGgucacug, GAGgucagaa, GAGgucagag, GAGgucagcu, GAGgucagga, GAGgucaggc, GAGgucaggg, GAGgucaggu, GAGgucagua, GAGgucauau, GAGgucaugu, GAGgucauuu, GAGguccaua, GAGguccauc, GAGguccggg, GAGguccggu, GAGguccuug, GAGgucgggg, GAGgucucgu, GAGgucugag, GAGgucuggu, GAGgucuguc, GAGgucuguu, GAGgucuuuu, GAGgugaaaa, GAGgugaaau, GAGgugaaca, GAGgugaagg, GAGgugaaua, GAGgugaauu, GAGgugacau, GAGgugacca, GAGgugaccu, GAGgugacua, GAGgugacuu, GAGgugagaa, GAGgugagac, GAGgugagag, GAGgugagau, GAGgugagca, GAGgugagcc, GAGgugagcg, GAGgugagcu, GAGgugagga, GAGgugaggc, GAGgugaggg, GAGgugagua, GAGgugagug, GAGgugaguu, GAGgugauau, GAGgugaucc, GAGgugaucu, GAGgugauga, GAGgugaugg, GAGgugaugu, GAGgugauuc, GAGgugcaca, GAGgugcaga, GAGgugcagc, GAGgugcagg, GAGgugccag, GAGgugccca, GAGgugccuu, GAGgugcggg, GAGgugcgug, GAGgugcucc, GAGgugcugg, GAGgugcuua, GAGgugcuug, GAGguggaaa, GAGguggaau, GAGguggacc, GAGguggacg, GAGguggagg, GAGguggcug, GAGgugggaa, GAGgugggag, GAGgugggau, GAGgugggca, GAGgugggcg, GAGgugggcu, GAGgugggga, GAGguggggc, GAGguggggg, GAGgugggua, GAGguggguc, GAGgugggug, GAGguggguu, GAGgugguau, GAGgugguuc, GAGgugucau, GAGgugugag, GAGgugugau, GAGgugugca, GAGgugugcu, GAGgugugga, GAGguguggg, GAGguguggu, GAGgugugua, GAGgugugug, GAGguuaaau, GAGguuaaga, GAGguuaaua, GAGguuaccg, GAGguuagaa, GAGguuagac, GAGguuagag, GAGguuaggu, GAGguuagua, GAGguuaguc, GAGguuagug, GAGguuaguu, GAGguuaugu, GAGguuauuc, GAGguucaaa, GAGguucaua, GAGguucuga, GAGguugaag, GAGguugcag, GAGguugcug, GAGguuggaa, GAGguuggag, GAGguuggau, GAGguuggua, GAGguugguc, GAGguugguu, GAGguuguag, GAGguuucug, GAGguuugag, GAGguuugga, GAGguuuggg, GAGguuugua, GAGguuuguu, GAGguuuuca, GAGguuuuga, GAGguuuugg, GAGguuuuua, GAGguuuuuc, GAUguaaaau, GAUguaagca, GAUguaagcc, GAUguaaggu, GAUguaagua, GAUguaagug, GAUguaaguu, GAUguacauc, GAUguaggua, GAUguauggc, GAUguaugua, GAUguauguu, GAUgucagug, GAUgugagag, GAUgugagcc, GAUgugagcu, GAUgugagga, GAUgugaguc, GAUgugagug, GAUgugaguu, GAUgugggua, GAUgugggug, GAUguguguu, GAUguuagcu, GAUguucagu, GAUguucgug, GAUguuuguu, GCAguaaagg, GCAguaagaa, GCAguaagga, GCAguaagua, GCAguaaguc, GCAguaaguu, GCAguagaug, GCAguaggua, GCAguaugug, GCAguauguu, GCAgucagua, GCAgucagug, GCAguccggu, GCAgugacuu, GCAgugagcc, GCAgugagcg, GCAgugagcu, GCAgugagua, GCAgugagug, GCAgugaguu, GCAgugggua, GCAguuaagu, GCAguugagu, GCCguaaguc, GCCgugagua, GCGguaaagc, GCGguaaaua, GCGguaagcu, GCGguaaggg, GCGguaagug, GCGguaauca, GCGguacgua, GCGguacuug, GCGguagggu, GCGguagugu, GCGgugagca, GCGgugagcu, GCGgugaguu, GCGguggcuc, GCGgugugca, GCGguguguu, GCGguuaagu, GCGguuugca, GCUgcuguaa, GCUguaaaua, GCUguaagac, GCUguaagag, GCUguaagca, GCUguaagga, GCUguaagua, GCUguaaguc, GCUguaagug, GCUguaaguu, GCUguaggug, GCUguauggu, GCUgucagug, GCUguccuug, GCUgugagaa, GCUgugagcc, GCUgugagga, GCUgugagua, GCUgugaguc, GCUgugagug, GCUgugaguu, GCUguggguu, GGAguaagag, GGAguaagca, GGAguaagcc, GGAguaagcu, GGAguaagga, GGAguaagug, GGAguaaguu, GGAguaauuu, GGAguacugu, GGAguaggaa, GGAguaggua, GGAguagguu, GGAguaguau, GGAguaugac, GGAguauggu, GGAgucaagu, GGAgugaggg, GGAgugagua, GGAgugaguc, GGAgugagug, GGAgugaguu, GGAgugcuuu, GGAgugggca, GGAgugggug, GGAguuaagg, GGAguugaga, GGCguaagcc, GGCguaggua, GGCguaggug, GGCgugagcc, GGCgugaguc, GGGguaaaca, GGGguaaacc, GGGguaaacu, GGGguaagaa, GGGguaagag, GGGguaagau, GGGguaagca, GGGguaagcc, GGGguaagcu, GGGguaagga, GGGguaaggg, GGGguaagua, GGGguaagug, GGGguaaguu, GGGguagaca, GGGguaggag, GGGguaggcc, GGGguaggga, GGGguaggua, GGGguaggug, GGGguagguu, GGGguagugc, GGGguaucug, GGGguaugac, GGGguaugga, GGGguaugua, GGGguauguc, GGGguaugug, GGGguauguu, GGGgucagua, GGGguccgug, GGGgucggag, GGGgucugug, GGGgugaaca, GGGgugaaga, GGGgugagaa, GGGgugagau, GGGgugagcc, GGGgugagcg, GGGgugagcu, GGGgugagga, GGGgugaggc, GGGgugaggg, GGGgugaguc, GGGgugagug, GGGgugaguu, GGGgugcgua, GGGguggggu, GGGgugggua, GGGgugggug, GGGguggguu, GGGgugugcg, GGGgugugua, GGGguguguc, GGGgugugug, GGGguuacag, GGGguuggac, GGGguuggga, GGGguuugcc, GGGguuugua, GGUguaagaa, GGUguaagau, GGUguaagca, GGUguaagcc, GGUguaagcg, GGUguaaguc, GGUguaagug, GGUguagguc, GGUguaggug, GGUguagguu, GGUguccgua, GGUgugagag, GGUgugagcc, GGUgugagcu, GGUgugagua, GGUgugaguc, GGUgugcuuc, GGUguggcug, GGUgugguga, GGUgugucug, GGUguugaaa, GGUguugcug, GUAguaagau, GUAguaagua, GUAguaagug, GUAguagcuu, GUAguaggua, GUAgucagua, GUAgugagua, GUAguggugg, GUAguuaagu, GUAguuucug, GUCguaagug, GUCgugagug, GUCgugaguu, GUGgcaagua, GUGgcuugua, GUGguaaaau, GUGguaaaga, GUGguaaauu, GUGguaacau, GUGguaacua, GUGguaagaa, GUGguaagac, GUGguaagag, GUGguaagau, GUGguaagca, GUGguaagcg, GUGguaagcu, GUGguaagga, GUGguaaggc, GUGguaagua, GUGguaaguc, GUGguaagug, GUGguaaguu, GUGguaauga, GUGguaauuc, GUGguaauuu, GUGguacaug, GUGguacgau, GUGguacuau, GUGguacuug, GUGguagaua, GUGguagcgc, GUGguaggga, GUGguagguc, GUGguaggug, GUGguagguu, GUGguauaaa, GUGguaucuc, GUGguaugaa, GUGguaugau, GUGguaugca, GUGguaugua, GUGguauguu, GUGguccgug, GUGgucuggc, GUGgugaaac, GUGgugagaa, GUGgugagau, GUGgugagca, GUGgugagcu, GUGgugagga, GUGgugaggc, GUGgugagug, GUGgugaguu, GUGgugauua, GUGgugauuc, GUGgugcgau, GUGgugcuua, GUGgugggaa, GUGgugggua, GUGguggguc, GUGguguccg, GUGguuagca, GUGguuaggu, GUGguuagug, GUGguuugca, GUGguuugua, GUUguaaggu, GUUguaagua, GUUguaaguc, GUUguaaguu, GUUguaccac, GUUguagcgu, GUUguaugug, GUUguauguu, GUUgucugug, GUUgugagcu, GUUgugagug, GUUgugaguu, GUUgugggua, GUUguggguu, UAAguaaaug, UAAguaacua, UAAguaagaa, UAAguaagag, UAAguaagau, UAAguaagca, UAAguaagcu, UAAguaagga, UAAguaaggu, UAAguaagua, UAAguaaguc, UAAguaagug, UAAguaaguu, UAAguaauaa, UAAguacuag, UAAguaguuu, UAAguauaaa, UAAguauaca, UAAguaugua, UAAguauuau, UAAguauuuu, UAAgucuuuu, UAAgugagac, UAAgugagga, UAAgugaggg, UAAgugagua, UAAgugaguc, UAAgugagug, UAAgugaguu, UAAgugaucc, UAAgugauuc, UAAgugcgug, UAAguuaagu, UAAguuccag, UAAguucuuu, UAAguuguaa, UAAguuguau, UAAguuuguu, UACguaacug, UACguaagaa, UACguaagau, UACguaagua, UACguaagug, UACguauccu, UACgucuggc, UACgugacca, UAGgcaagac, UAGgcaaguc, UAGgcagguc, UAGgcgugug, UAGguaaaaa, UAGguaaaac, UAGguaaaag, UAGguaaaau, UAGguaaaca, UAGguaaaga, UAGguaaaua, UAGguaaauc, UAGguaaaug, UAGguaaauu, UAGguaacac, UAGguaacag, UAGguaacau, UAGguaacca, UAGguaacgg, UAGguaacua, UAGguaacuc, UAGguaacug, UAGguaacuu, UAGguaagac, UAGguaagag, UAGguaagau, UAGguaagca, UAGguaagcc, UAGguaagcu, UAGguaagga, UAGguaaggc, UAGguaaggg, UAGguaagua, UAGguaaguc, UAGguaagug, UAGguaaguu, UAGguaauag, UAGguaauau, UAGguaaucu, UAGguaauga, UAGguaaugg, UAGguaaugu, UAGguaauua, UAGguaauuc, UAGguaauuu, UAGguacagc, UAGguacagu, UAGguacauu, UAGguaccag, UAGguaccua, UAGguaccuu, UAGguacgag, UAGguacgua, UAGguacguu, UAGguacuau, UAGguacuga, UAGguacugg, UAGguacuuc, UAGguacuuu, UAGguagcgg, UAGguaggaa, UAGguaggac, UAGguaggau, UAGguaggga, UAGguagggg, UAGguaggua, UAGguagguc, UAGguaggug, UAGguagguu, UAGguaguaa, UAGguagucu, UAGguagugg, UAGguagugu, UAGguaguuu, UAGguauaaa, UAGguauaac, UAGguauaag, UAGguauaau, UAGguauaca, UAGguauacu, UAGguauaua, UAGguauauc, UAGguauauu, UAGguaucag, UAGguaucua, UAGguaucuc, UAGguaugaa, UAGguaugag, UAGguaugca, UAGguaugga, UAGguauggc, UAGguauggu, UAGguaugua, UAGguauguc, UAGguaugug, UAGguauguu, UAGguauuaa, UAGguauuac, UAGguauuau, UAGguauuca, UAGguauucc, UAGguauucu, UAGguauuga, UAGguauuua, UAGguauuuc, UAGguauuuu, UAGgucacuc, UAGgucagcu, UAGgucaggu, UAGgucagua, UAGgucagug, UAGgucaguu, UAGgucaucu, UAGgucauug, UAGguccaau, UAGguccugu, UAGgucucaa, UAGgucucgc, UAGgucuggc, UAGgucuguc, UAGgucugug, UAGgugaagu, UAGgugaaua, UAGgugaaug, UAGgugaauu, UAGgugacau, UAGgugacca, UAGgugacua, UAGgugagaa, UAGgugagac, UAGgugagag, UAGgugagau, UAGgugagcc, UAGgugagcu, UAGgugagga, UAGgugaggc, UAGgugaggu, UAGgugagua, UAGgugaguc, UAGgugagug, UAGgugauca, UAGgugauuc, UAGgugauuu, UAGgugcaua, UAGgugcauc, UAGgugccgu, UAGgugccug, UAGgugcgca, UAGgugcgua, UAGgugcgug, UAGgugcuga, UAGguggaua, UAGgugggaa, UAGgugggac, UAGgugggag, UAGgugggau, UAGgugggcc, UAGgugggcu, UAGguggguu, UAGguggugu, UAGguguaaa, UAGgugugaa, UAGgugugag, UAGgugugca, UAGgugugcc, UAGgugugcg, UAGguguggu, UAGgugugua, UAGgugugug, UAGguguugg, UAGguuaagc, UAGguuagac, UAGguuagcc, UAGguuaggc, UAGguuagua, UAGguuaguc, UAGguuagug, UAGguucccc, UAGguucuac, UAGguuggua, UAGguugguu, UAGguugucc, UAGguuuauu, UAGguuugcc, UAGguuugua, UAGguuuguc, UAGguuugug, UAGguuuguu, UAGguuuuuc, UAGguuuuug, UAUguaagaa, UAUguaagau, UAUguaagca, UAUguaagcc, UAUguaagua, UAUguaaguc, UAUguaagug, UAUguaaguu, UAUguacgug, UAUguacguu, UAUguagguc, UAUguagguu, UAUguauccu, UAUguaucuc, UAUguaugua, UAUguauguc, UAUguaugug, UAUguauuau, UAUgucagaa, UAUgucugua, UAUgugaaua, UAUgugacag, UAUgugagua, UAUgugagug, UAUgugaguu, UAUgugggca, UAUgugugua, UAUguguuua, UAUguuuugu, UCAgcgacau, UCAguaaaau, UCAguaaaua, UCAguaacug, UCAguaagaa, UCAguaagag, UCAguaagau, UCAguaagca, UCAguaagcc, UCAguaagcu, UCAguaaggg, UCAguaagua, UCAguaaguc, UCAguaagug, UCAguaaguu, UCAguaucuu, UCAguaugga, UCAguauggu, UCAgucccca, UCAgugagca, UCAgugagcu, UCAgugagua, UCAgugagug, UCAgugaguu, UCAgugauug, UCAgugggug, UCAguugagc, UCAguugauu, UCAguuuagu, UCCguaagca, UCCguaagcu, UCCguaaguc, UCCguaagug, UCCguaauag, UCCguacuua, UCCguaugua, UCCguauguu, UCCgugagau, UCCgugaguc, UCGguaaauu, UCGguaagag, UCGguaagcu, UCGguacauc, UCGguacucc, UCGguagacc, UCGguagguu, UCGguaguaa, UCGguaugug, UCGguauguu, UCGguauuga, UCGgucagua, UCGgucuuag, UCGgugaagu, UCGgugagaa, UCGgugagca, UCGgugaggc, UCGgugagua, UCGgugcgcu, UCGgugcuuu, UCGgugguuu, UCGguuagcu, UCUguaaaag, UCUguaagaa, UCUguaagau, UCUguaagca, UCUguaagcu, UCUguaagua, UCUguaaguc, UCUguaagug, UCUguaaguu, UCUguaauaa, UCUguaauga, UCUguaaugu, UCUguaggua, UCUguagguu, UCUguauaua, UCUguaugac, UCUguaugua, UCUguccucg, UCUgugagag, UCUgugagcu, UCUgugagga, UCUgugagua, UCUgugaguc, UCUgugagug, UCUgugaguu, UCUgugcgua, UCUgugugag, UGAguaacuu, UGAguaagau, UGAguaagca, UGAguaagcu, UGAguaaggc, UGAguaaggu, UGAguaagua, UGAguaaguc, UGAguaagug, UGAguaaguu, UGAguaaucc, UGAguaauua, UGAguacagu, UGAguacgua, UGAguacguu, UGAguacugu, UGAguagcug, UGAguaggua, UGAguauaaa, UGAguaugcu, UGAguaugga, UGAguaugua, UGAguauguc, UGAguauguu, UGAgucagag, UGAgucuacg, UGAgugaaua, UGAgugaauu, UGAgugagaa, UGAgugagau, UGAgugagca, UGAgugagcc, UGAgugagga, UGAgugagua, UGAgugagug, UGAgugaguu, UGAgugggaa, UGAguuaaga, UGAguuaaug, UGAguuacgg, UGAguuaggu, UGAguucuau, UGAguugguu, UGAguuguag, UGAguuuauc, UGCguaaguc, UGCguaagug, UGCguacggc, UGCguacggg, UGCguaugua, UGGgcaaguc, UGGgcaagug, UGGgcacauc, UGGgccacgu, UGGgccccgg, UGGguaaaau, UGGguaaagc, UGGguaaagg, UGGguaaagu, UGGguaaaua, UGGguaaaug, UGGguaaauu, UGGguaacag, UGGguaacau, UGGguaacua, UGGguaacuu, UGGguaagaa, UGGguaagac, UGGguaagag, UGGguaagau, UGGguaagca, UGGguaagcc, UGGguaagcu, UGGguaaggg, UGGguaaggu, UGGguaagua, UGGguaaguc, UGGguaagug, UGGguaaguu, UGGguaaugu, UGGguaauua, UGGguaauuu, UGGguacaaa, UGGguacagu, UGGguacuac, UGGguaggga, UGGguagguc, UGGguaggug, UGGguagguu, UGGguaguua, UGGguauagu, UGGguaugaa, UGGguaugac, UGGguaugag, UGGguaugua, UGGguauguc, UGGguaugug, UGGguauguu, UGGguauuug, UGGgucuuug, UGGgugaccu, UGGgugacua, UGGgugagac, UGGgugagag, UGGgugagca, UGGgugagcc, UGGgugagga, UGGgugaggc, UGGgugaggg, UGGgugagua, UGGgugaguc, UGGgugagug, UGGgugaguu, UGGgugcgug, UGGguggagg, UGGguggcuu, UGGguggggg, UGGgugggua, UGGguggguc, UGGgugggug, UGGguggguu, UGGgugugga, UGGguguguc, UGGgugugug, UGGguguguu, UGGguguuua, UGGguuaaug, UGGguuaguc, UGGguuagug, UGGguuaguu, UGGguucaag, UGGguucgua, UGGguuggug, UGGguuuaag, UGGguuugua, UGUgcaagua, UGUguaaaua, UGUguaagaa, UGUguaagac, UGUguaagag, UGUguaaggu, UGUguaagua, UGUguaaguc, UGUguaaguu, UGUguacuuc, UGUguaggcg, UGUguaggua, UGUguaguua, UGUguaugug, UGUgucagua, UGUgucugua, UGUgucuguc, UGUgugaccc, UGUgugagau, UGUgugagca, UGUgugagcc, UGUgugagua, UGUgugaguc, UGUgugagug, UGUgugcgug, UGUgugggug, UGUguggguu, UGUgugugag, UGUguguucu, UGUguuuaga, UUAguaaaua, UUAguaagaa, UUAguaagua, UUAguaagug, UUAguaaguu, UUAguaggug, UUAgugagca, UUAgugaguu, UUAguuaagu, UUCguaaguc, UUCguaaguu, UUCguaauua, UUCgugagua, UUCgugaguu, UUGgcaagug, UUGgccgagu, UUGguaaaaa, UUGguaaaau, UUGguaaaga, UUGguaaagg, UUGguaaagu, UUGguaaauc, UUGguaaaug, UUGguaaauu, UUGguaacug, UUGguaacuu, UUGguaagaa, UUGguaagag, UUGguaagcu, UUGguaagga, UUGguaaggg, UUGguaagua, UUGguaagug, UUGguaaguu, UUGguaauac, UUGguaauca, UUGguaaugc, UUGguaaugu, UUGguaauug, UUGguaauuu, UUGguacaua, UUGguacgug, UUGguagagg, UUGguaggac, UUGguaggcg, UUGguaggcu, UUGguaggga, UUGguaggua, UUGguagguc, UUGguaggug, UUGguauaaa, UUGguauaca, UUGguauauu, UUGguaucua, UUGguaucuc, UUGguaugca, UUGguaugua, UUGguaugug, UUGguauguu, UUGguauugu, UUGguauuua, UUGguauuuu, UUGgucagaa, UUGgucagua, UUGgucucug, UUGgucugca, UUGgugaaaa, UUGgugacug, UUGgugagac, UUGgugagau, UUGgugagca, UUGgugagga, UUGgugaggg, UUGgugagua, UUGgugaguc, UUGgugagug, UUGgugaguu, UUGgugaugg, UUGgugauua, UUGgugauug, UUGgugcaca, UUGgugggaa, UUGguggggc, UUGgugggua, UUGguggguc, UUGgugggug, UUGguggguu, UUGguguggu, UUGguguguc, UUGgugugug, UUGguguguu, UUGguuaagu, UUGguuagca, UUGguuagug, UUGguuaguu, UUGguuggga, UUGguugguu, UUGguuugua, UUGguuuguc, UUUgcaagug, UUUguaaaua, UUUguaaaug, UUUguaagaa, UUUguaagac, UUUguaagag, UUUguaagca, UUUguaaggu, UUUguaagua, UUUguaaguc, UUUguaagug, UUUguaaguu, UUUguaauuu, UUUguacagg, UUUguacgug, UUUguacuag, UUUguacugu, UUUguagguu, UUUguauccu, UUUguauguu, UUUgugagca, UUUgugagug, UUUgugcguc, UUUguguguc, and uGGguaccug.

Additional exemplary gene sequences and splice site sequences (e.g., 5′ splice site sequences) include AAGgcaagau, AUGguaugug, GGGgugaggc, CAGguaggug, AAGgucagua, AAGguuagag, AUGgcacuua, UAAguaaguc, UGGgugagcu, CGAgcugggc, AAAgcacccc, UAGguggggg, AGAguaacgu, UCGgugaugu, AAUgucaguu, AGGgucugag, GAGgugacug, AUGguagguu, GAGgucuguc, CAGguaugug, CAAguacugc, CACgugcgua, CCGgugagcu, CAGguacuuc, CAGgcgagag, GAAgcaagua, AGGgugagca, CAGgcaaguc, AAGgugaggc, CAGguaagua, CCAguugggu, AAGguguggg, CAGguuggag, CCGguaugaa, UGGguaaugu, CAGgugaggu, AGAguaauag, CAGguaugag, AUGguaaguu, UUGguggguc, UUUguaagca, CUCguaugcc, UAGguaagag, UAGgcaaguu, GGAguuaagu, GAGguaugcc, AAGguguggu, CAGgugggug, UUAguaagua, AAGguuggcu, UGAguaugug, CCAgccuucc, CCUguacgug, CCUguaggua, CAGguacgcu, GAGguucuuc, AAGguugccu, CGUguucacu, CGGgugggga, UAGgugggau, CGGguaagga, AAGguacuau, GGGguaagcu, ACGguagagc, CAGgugaaga, GCGguaagag, CAGguguugu, GAAguuugug, AUGgugagca, CGGguucgug, AUUguccggc, GAUgugugug, AUGgucuguu, AAGguaggau, CCGguaagau, AAGguaaaga, GGGgugaguu, AGGguuggug, GGAgugagug, AGUguaagga, UAGguaacug, AAGgugaaga, UGGguaagug, CAGguaagag, UAGgugagcg, GAGguaaaaa, GCCguaaguu, AAGguuuugu, CAGgugagga, ACAgcccaug, GCGgugagcc, CAGguaugca, AUGguaccua, CAAguaugua, AUGguggugc, UAAguggcag, UAGguauagu, CUGguauuua, AGGguaaacg, AUAguaagug, UUGguacuga, GGUguaagcc, GAGguggaua, GAUguaagaa, ACGgucaguu, UAAguaaaca, AAGguaucug, AGGguauuug, AAGgugaaug, CUGgugaauu, CAGguuuuuu, CAUguaugug, UUGguagagg, AAGguaugcc, CAGgugccac, UCGguauuga, AAGguuugug, AAUguacagg, CAUguggguu, CAUgugaguu, UUGguaaugu, AGUguaggug, GAGguaacuc, GAGguggcgc, CUGguaauug, GAGguuugcu, UGUguacgug, UAGguaaaga, CUAguaggca, UCUgugaguc, UCUguaaggc, CAGguuugug, GAGguagggc, AAGguaacca, ACUgugaguu, UAGguaauag, AAAguaagcu, AUGgugagug, UAGguuugug, AACguaggac, GUAgcaggua, GAGgucagac, AGGguaugaa, GAGguuagug, CAGgcacgug, GGGgcaagac, CAGguguguc, CAGguauuga, CAGguauguc, AAGgcaaggu, UUGgugagaa, AAGguaaaau, GGGguaagua, AAGguaucuu, GACgugaguc, UAUguaugcu, AAGguacugu, CAGgugaacu, CACguaaaug, AAGgugugau, GAAguauuug, AAGgucugug, AAGguggagg, AAGguauaug, CAGguucuua, AGGguaacca, CAGgugucac, AAAguucugu, UUGgugaguu, CAAgugaguc, UAGguagguc, GCGgugagcu, AUUgugagga, CAGgugcaca, CAGguuggaa, CUGgucacuu, GGAguaagug, GAGgugggcu, AAGguacuug, AGGguaggau, AAUguguguu, ACAguuaagu, GAGgugugug, AAGgcgggcu, AUAgcaagua, AAGguuguua, CAAgcaaggc, GUGguaauua, UCUguucagu, AGGguaggcc, AAGguaucau, UAGguaccuu, AAGguaugac, GGAguaggua, UAAguuggca, AGUgugaggc, GAGguuugug, UGGgucugcu, CAGgugaucc, CAGgucagug, AAGguaaggg, CAGgugcagu, GAGguggguc, GCUgugagug, AAGguggagu, GGGgucaguu, AGCguaagug, AGAguaugaa, GGGguagggu, AAGgccagca, CGAguaugcc, GUGgugagcg, AAUguaaauu, CAGgugcgca, GGUguaugaa, CUUgugaguu, AAGguaucuc, AGAguaagga, UAGguaagac, GAGgugagug, CAGguguguu, UUGgugagua, AGGgcgaguu, CAGguuuugc, UUUgugaguu, AGGguaagca, GAGguccucu, CCAgcaggua, GAGguucgcg, CAGgugaucu, ACUguaagua, AAGguaaauc, CAGgcaaaua, GUGguaagca, CAGguuaaau, UUGguaauaa, UAUguaggua, CAGguaguau, AAGgugugcc, UGGguaagag, CAGgcaagca, UUGguaaggg, AAGgcaggug, ACGguaaaug, GCUgugagca, AUGguacaca, GUAguguguu, ACUguaagag, CCCgcagguc, GAGgugagcc, GAGgugcugu, UAAguaugcu, GAGgccaucu, UCAgugagug, CAGgugcuac, AAUgugggug, GAGgugugaa, CUGguagguc, GUGgcgcgcg, CAGgugcaaa, UAAguggagg, CAUgugggua, GAGguagggu, AAAgugaguu, AGGguucuag, UGUgugagcu, AGGgugaauc, CAGgucaggg, AAGgucccug, CUGguagagu, UAGgucaguu, AAAguaaggg, CAAguaugug, CAGgugcuuu, AAGguaauuc, GGGgugcacg, ACUgugcuac, CAGguaccua, CAGguagcuu, UGGgugaggc, CUGguacauu, AGGguaaucu, CAGguacaag, CAGguaauuc, AGGgcacuug, UAGgugagaa, GAGguaaugc, CCAgugaguu, AAAguaugug, CUGgugaauc, UAUguaugua, CCUgcaggug, CAGguaucug, GAGgugaggu, CUGguaaaac, UGUgugugcu, CAGguuaagu, CAGguaaucc, UAGguauuug, UGGguagguc, CAGguaacag, AGCgugcgug, AAGgucagga, GGUgugagcc, CUGguaagua, GGGgugggca, AAGgugggaa, CAGgugagug, CUGguuguua, CAGguaauag, UAGgugaguu, AGAguaaguu, UAGguaaucc, CCGgugacug, GUCgugauua, CUUguaagug, UAGguaguca, CUGguaaguc, AGGgugagcg, CAGguaugga, AUUgugacca, GUUgugggua, AAGguacaag, CUAgcaagug, CUGgugagau, CAGgugggca, AUGgcucgag, CUGguacguu, UUGgugugua, GAGgugucug, GAGgugggac, GGGgugggag, GCAgcgugag, GAGguaaaga, GAGguaugua, AAGgugagac, AAGguacaau, CUGguaugag, AACguaaaau, GUGguaggga, CUGguaugug, CUUguaagca, AAGguaggga, AUUguaagcc, AUGguaagcu, CAGgugaauu, UAGgugaaua, CAAguaugga, AUGguauggc, GAGgucaugc, CAGguacccu, ACAgugagac, CAGgucugau, GAAguugggu, CUGgugcgug, CAGguacgag, ACAgugagcc, AAGguaagua, GGAguaaggc, GAGgugugua, AAGgucauuu, CAGguagucu, AUGguaucug, AAGguaaacu, GAGguaggug, CUGguaagca, AGGguaagag, AAAguaaagc, CAGguuugag, GAGgcgggua, CGAguacgau, CAGguuguug, AAAguauggg, UAGgcugguc, AAGguaagga, AAGguuuccu, UUGguaaaac, GAGguaagua, CAGguucaag, UGGguuaugu, GAGgugaguu, ACGgugaaac, GAUguaacca, AAGgugcggg, CCGguacgug, GAUgugagaa, GUGgcgguga, CAGguauuag, GAGguuggga, AAGgcuagua, AAGgugggcg, CAGgcaggga, AAUguuaguu, GAGguaaagg, CAGgugugcu, CUGguaugau, AUGguuaguc, CUGgugagaa, CAGgccggcg, CAGgugacug, AAAguaaggu, UAAguacuug, AAGguaaagc, UCGguagggg, CAGguaggaa, AGUguaagca, CCCgugagau, GUGguuguuu, CAGguuugcc, AGGguauggg, UAAguaagug, GAGguaagac, GAUguagguc, CAAguaggug, AUAguaaaua, GAGguugggg, GAGgcgagua, CAGguagugu, GUGguaggug, CAAgugagug, AAGgugacaa, CCAgcguaau, ACGgugaggu, GGGguauauu, CAGgugagua, AAGgugcgug, UAUguaaauu, CAGgucagua, ACGguacuua, GAGgucagca, UAAguaugua, GGGgucagac, AAUgugugag, UCCgucagua, CAGgugcuuc, CCAguuagug, CCGgugggcg, AGGgugcaug, GGGguaggau, UAGgugggcc, GAGguguucg, UUGgcaagaa, UCCguaagua, CAGguguaag, CUCgugagua, GAGguguuuu, GAGgugagca, GAGguaaagu, AAGguacguu, CAGguccagu, AUGgugaaac, GUAgugagcu, CAGgugaaaa, AGGguacagg, AAGguaacgc, AAGguauacc, CCUgugagau, GGGguacgug, GAGguauggu, UAGguauuau, GAAguaggag, UCGguaaggg, CCGguaagcg, GAAguaauua, CAGgugaguc, AAGgucaaga, AUGguaaguc, CAGgugagcu, CCAguuuuug, CAGgugggag, AAGguauuau, AAGguaaaua, AAGgugcugu, AAAguacacc, CUGguucgug, UCAguaaguc, GAAguacgug, CAGgugacaa, UGGguaagaa, UGUguagggg, GAGguaggca, UUGgugaggc, AUGgugugua, CAGguccucc, UUGguaaaug, GCUgugaguu, AUGgucugua, CAUgcaggug, CUGguacacc, CAGguccuua, CAAguaaucu, AUGgcagccu, AAGgucagaa, AACgugaggc, CAGgcacgca, ACGguccagg, UCUguacaua, GAGgugauua, ACGguaaaua, AUGguaacug, CAGgcgcguu, CAGguauaga, AAGguuuguu, CAGguaugaa, UAGguuggua, CUGgugagac, CAGguuagga, AUGgugacug, UUGguauccc, CUUguaggac, AAAguguguu, CAGguuucuu, GGGguauggc, GGGguaggac, ACUguaaguc, AUCguaagcu, UAGguucccc, GGUgugagca, CUGguuggua, GGGguuaggg, UGAguaagaa, GAGguauucc, UGGguuaguc, CAGgcucgug, UAGguagagu, UAGgugcccu, AAAgugagua, GAGguucaua, UUGguaagag, ACCgugugua, UAUguaguau, UGGguaauag, CAGgucugaa, AAAguauaaa, GUGgugaguc, AGUgugauua, UUGgugugug, CAGgugaugg, GCUgugagua, CAGguacaug, AAGguacagu, GAAguuguag, CAGgugauua, UAGgugaauu, GGUguuaaua, CAGguauuua, CAAguacucg, CAAguaagaa, AAGguaccuu, ACGgugaggg, UGAgcaggca, GGGgugaccg, GAGguaaaug, CGGguuugug, AAGgugagcg, GUGguaugga, CUGguaagga, GAGguaccag, CCGgugagug, AAGguuagaa, GAGguacuug, AGAguaaaac, UCUgugagua, AAGgcgggaa, CAGguaugcg, AGGguaaaac, AAGgugacug, AGGguauguu, AAGguaugua, CAGgucucuc, CAGgcaugua, CUGguaggua, AAGgucaugc, CAGguacaca, GAUguacguu, ACAguacgug, ACGguaccca, CAGguagugc, ACAguaagag, GGUgcacacc, GAGguguaac, AAGgugugua, UAGguacuua, GCGguacugc, UGGguaaguc, CAUguaggua, CAGguaggau, CAGgucuggc, GUGguuuuaa, CAGgugggaa, UGGgugagua, CGAgugagcc, AAGguauggc, AGUguuguca, CAGgugauuu, UAGguaucuc, UAAguauguu, AAGguugagc, AGAguaaaga, GGUguaagua, GGGgugagcu, CAGguauaau, GAGguacaaa, AUGguaccaa, UAGguagggg, UGAgucagaa, AAGgcaauua, UUGguaagau, CAGguacaga, AGAguuagag, CAGgugcguc, GAGguauuac, ACGguacaga, CAGgucuucc, AAGguaaggu, GAGguaauuu, AGUguaggcu, AAAguaagcg, CCUguaagcc, AGGgugauuu, UGUguaugaa, CUGguacaca, AGGguagaga, AUAguaagca, AGAguaugua, UUGgucagca, CAGgcaaguu, AAGguauaua, AAGgucugga, CAGguacgca, AGGgugcggg, AUGguaagug, AAAgugauga, UGCgugagua, AGAguaggga, UGUguaggua, UAGguaggau, UAAgugagug, GCUguaagua, GAAguaagaa, UCGgugaggc, UAGguauuuu, AAGguacaca, AAGguaggua, UGGguagguu, ACAgcaagua, GAGguaggag, UGGgugaguu, GCGgugagau, CCUguagguu, CAGgugugua, CUGguaagcc, AAGgugauuc, CAGguagcua, GUUguaagug, AUGguaagca, AUAguaggga, GGGguucgcu, CCGgucagag, GUAguaugag, CGUguaagau, UGAguaggca, UCAguaugua, GAGguaucug, AGAguauuuu, AAGguuguag, AGUguaaguu, CGGguaaguu, UCGgugcgga, UAGguaagua, GAAguuagau, GCUgugagac, CAGgcaggua, CAGguagggg, UAAguuaaga, AUGguggguu, UAGguaaguu, CUGguaaauu, CCGguaagga, GAGgcaggca, CAUguaagug, AAGgugccua, UUGguaggga, AAGguaaaca, CGGgugugag, GGGgugugag, UCCguggguc, ACGguaaauc, UCAguaggua, CAGgucagcc, CAGgcggugg, CGAguaagcu, CCCgugagca, AAAguaauga, CUGguaagcu, CGGguaacca, CAGgucgcac, GAGguaggcc, UAGgugagcc, UAGguaggca, GCGgugcgug, AUGgugagua, GGGgugaggg, GAGgucacac, CAGguaggcc, CAAgugcuga, GUCgucuuca, CAUguaagaa, GUAguaagga, UAGguuugua, CAAguuagag, AAGguagagu, AAGgugagau, AAAguaggua, ACAgugaauc, CAGgugugcg, CAGgucggcc, AAGguaguau, ACUgucaguc, UCUgcagccu, CGAguaagug, AGAguaauua, AGUgugagug, CCGgugagcg, AAGguaaccu, AAGguugugg, AAGgcauggg, AAGgucagag, ACGguaaggu, GGGgugagca, GAGguugcuu, AAGguaucgc, CCGguaaagg, AAAguuaaug, UAGguacgag, ACCguaauua, GGGguaagga, CCGguaacgc, CAGgucagaa, AAGguacuga, GAGgugacca, GGGgugagcc, AAGguacagg, AUGguaauua, CAGgugagag, AAGgugacuc, AUAguaagua, GAGguaaacc, CAGgugggau, CAGgugagaa, AGGguaaaaa, GAGgugugac, CACguaagcu, CAGguccccc, CAGgucaggu, CGGguaaguc, ACGguauggg, GAUguaaguu, CAAguaauau, CAGguugggg, CCUgugcugg, AAGguaugau, AGGguagagg, AAGguggguu, CAGgugugaa, UUGguaugug, UUGguaucuc, GGGgugagug, CUGgugugug, AGGguagggc, GUGgugagua, CAGguaugua, AAGguacauu, UUAguaagug, AAUguauauc, CUUguaagua, GAGguuagua, CAGguaaggu, CAGguaaugu, AGGgugaggc, CAGguauuuc, CAGgucugga, GGGgugugcu, UAGgugagug, AAUguaaccu, UAAgugaguc, CAGgugcacu, ACGguaagua, GAGguauccu, UCUguaaguc, CAGguauuca, UGUguaagug, CCAgcaaggc, GAGgugaagg, AAUguggggu, UCGgugcgug, UUGguaaggc, GAGguaagug, AAAguaagau, UAGgucuuuu, GAGgucugau, CCAguuagag, UGGgugaaaa, AGAguaagau, CAGguaauug, CAGgccgguc, CCGguaagag, GAGgugagcu, CUGguaagac, CAGgugagau, CUGguuuguu, UGGguaggua, CAGguuagug, CAGguguucg, CGGguagguc, GUGguacaua, AAGguacuaa, GAUgugagua, UGUguaagac, GAGguagccg, UAGgugaucu, CAGguacgug, CUUgucaguc, GAGguaucac, GAGguaauga, AAGguaacac, CAGguaaagc, AAGgcaagua, CGCgugagcc, AGUgugcguu, GAUguaagca, AAGguaauag, GGAgcaguug, AGCguaagau, AAGgucaggc, GAGguauuca, AAUguaaagu, CAGguaacaa, UCGguaggug, AAAguaaguc, CGGgugcagu, GGUgugugca, UGAgugagaa, CACguguaag, GUGguuggua, GCAgccuuga, CGAgugugau, CAGguauaua, UAUguaugug, CCCgugguca, AUGguaagac, GAGgugugga, AGUguauccu, UGAguguguc, UGGguaaucu, AUGgcagguu, GAGguaagau, UCAgcagcgu, AAGgugggau, CGGgugcgcu, CAGgugucug, AGCgugguaa, AAUgugaaug, UCGgugagac, UAGguaaagc, CUGguaaaag, CCGgugcgga, CAGguacuca, CAGguagcaa, GAAguugagu, GAGguggagg, AGGguaugag, UAGguaugcu, UAGgugagac, CAGguaauua, CGUguaagcc, CUUguaaguu, AAGguaacuu, UCGgcaaggc, GAGguucucg, GAGgugggcg, AAGgcaugug, CUGguauguu, UAAgucauuu, CAUguaauua, AAUguaaaga, UAGgugcuca, AAGguaaugg, GAGguacuga, UGGguaagua, UGGguaaaaa, AAGgugagcu, UACgugaguu, AGGgugagcc, CGGgugagga, UGGgugagag, GGUguaagcu, CGGguggguu, CCAgcuaagu, AAGguuuguc, GAGguuagac, GAGguaccuc, UUUguaaguu, GAGguuagga, CAGguaggga, AGGguaauac, UGCgugugua, CCAguaacca, AGGgucuguc, UGGguaugua, GUGguaagcu, CAGguaaccu, AAGgugaguu, UAGguucgug, AAAguuagua, UGGgcaaguc, AAGgcacagu, GUUguaaguc, AAGguuugcc, CUUgcauggg, GCGgugagua, GGGguaagcg, GCCguaagaa, GAGgucggga, UUGguauugu, AGUgugagac, CUGgugggga, AGAguaaggu, CCGguggguc, CAGguauucu, UGGguaacgu, UUGgugagag, UAGguacccu, GGGgugcguc, AAGgcaggag, ACGguacauu, GAGguaguua, CAGguauggg, UUUguguguc, CAGguacuua, AUGguauacu, AGUgugagcc, ACAguaacga, CUGguaccca, CAGguaaccc, GGAguaagua, GAGgugggug, ACUguauguc, ACGgugagua, CUGguaaugu, AAGguaucag, CAGgugcccc, AGUgucagug, AAGguaggag, GGAguaugug, UUGguauuuu, CCUguuguga, UUUguaagaa, UAGguaacau, CAGguaagca, CAGgucacag, CAGgugugag, UAGguuugcg, CUGguaagaa, ACGguuguau, AAGguugggg, AAGgugaauu, GGGguuaguu, ACGguaaggc, CAGguuuaag, CUGguaaguu, GGGgugagag, UGGguggguu, GAGguuuguu, UGGguaaaug, CAGgcaggcc, CACgugcagg, AAGgugagcc, CAAguaagug, CAGgucaguc, GCGguauaau, UAGguaaagu, UAGguggauu, GAGgucugga, UCGgucaguu, UGGguaacug, AAGguuugau, UGUgcuggug, UGUguaccuc, UGGguacagu, AUCgucagcg, CAGgucuugg, GAAguuggua, GAAguaaaga, UUGguaagcu, UAGguaccag, AGGguaucau, CAGguaaaaa, ACGguaauuu, AUUguaaguu, GAGguacagu, CAGgugaaag, UGGguuguuu, GGGguaggug, CAGgugccca, AGCgugagau, CCAgugagug, AGGguagaug, UGGguguguc, AUCgcgugag, AGGguaagcc, AGGguagcag, UUCguuuccg, AAGguaagcg, UGGguaagcc, CAGguauggc, UGUguaagua, AAGguagaga, ACGguaauaa, CUGguacggu, GAGgucacag, UAUguaaguu, CUGguacgcc, CAAguaagau, CUAgugagua, CCGguaaccg, CUUguaaguc, GUGgugagaa, ACCguaugua, GUAguaagug, UUGgugggua, CGGguacuuu, UGGguaaaua, AGAgugagua, AAGguagguu, AAGguaugcg, CCUguaggcu, ACAguagaaa, CCGguuagua, CGGguaggcg, GCAgugagug, GAGgugaguc, CUGguagccu, CAUguaugua, GAAguaacuu, GAAguaagau, AAGguuagau, AAGguaauca, AAUguaugua, UGAguaagau, AGAgugagca, GUAguucuau, GAGguaauca, UAGguaugga, UAGgugggac, GAGguacaug, UGGguaaggc, CAGguacgcc, CCAguuacgc, ACUgugguga, GAGguaaguc, AUUguaggug, ACCgucagug, AAUgugaggg, ACUgugagug, UGGguguggu, AAGguuggga, AAGguuugga, UCCgugagug, CGGgugagug, AGAguaagcu, CAGgcaagcu, UAGguauauu, AAAguagcag, GAGguaaccu, AAGgugggca, AGGgugagua, UGGguaaggu, CUUgucagug, UAGgugcgcu, GAGgcaaauu, AGGguaccuc, CAAgugcgua, AGAguaagac, GUGguaaaua, GAUguaagcg, GAGguaaagc, UAGgugagua, CAGguaacau, CCUguacggc, UAGguauguc, UAGguccaua, GAGgugaaaa, AAAguacuga, UUGguaagcg, CAGgcaagcg, UUUgcagguu, CAGguuuaua, CUGguaaagc, AUGgugagcu, CAGgugguug, GUAguaaguu, CAGguaauac, CAGgcaaggc, AAGguaauuu, UUUguccgug, GAGguagguu, ACCgugagug, CAAguaagcu, ACAgugagua, UUGgugagau, AAGguagucu, CAGguaaagg, GGGguaugga, UUUguaagug, GUGguaagag, AGUgugaguu, AAGgcaagcg, UAAgugagua, AGGgugagug, AGUguacgug, AGGgugcgua, GGCgugagcc, CGAguuauga, CAGguaaaga, UUGgugaaga, AGGguaaugg, AAGguccaga, AGUgugaguc, CAGguaauuu, CAGguaacgc, CUGguacacu, CUGguuagug, CAGguacuug, CACguaagua, GUGgugcggc, GAGgucaguu, AUGguaugcc, AAGgugugug, CUGguggguc, CAGgugaggc, AAGguuaguc, AAGguagcug, GAGgucagga, GUUguaggua, UGGguacaag, AUGguaggug, GAGguaagcc, AUGgcaagua, AAGguauauu, GCGgugagag, AAGgugcuuc, UAGguacauc, ACUgugguaa, GAGguaggcu, GAGguaugca, AGGguaguuc, CAGguauccu, AGGguaaguc, AGGgucaguu, CAGguuggga, CAGguggaua, GGAguagguu, GAGguaggau, GGGguuugug, UAGguaauug, AAGguaaccc, ACGguaagaa, GAGguagggg, CGAguaggug, UCCguaagug, UCGguacagg, CAAguaagcg, AAGguccgcg, AAUgugagua, CAGgugaaug, GUGguaaggc, AGAgugagug, UCUguauguc, UGGgugaguc, UCGguuagua, GAUguaugca, GAGguuggug, GAGguggggc, UGGgucaguc, GCAgugagua, CAGguugcuu, AGGguagagu, UAGgucaggu, CGCguaugua, GAGguauuaa, CAGguaaacu, AAAguaaguu, GGGgucuggc, GCUguggggu, UUGguaaguc, AAGguagaag, AAUgugaguc, AAGgucagcu, AAGguaagag, AUGgugagga, AAGguacuuc, AAGguaagaa, CCGguacagc, GCGgugcgga, CAGguacaua, CUGgugagga, CUGguaggug, AACguagguu, AUGgugugug, UUGguacuau, CAGgucggug, CAGgcauggg, AUGguaucuu, AAGguaacua, CAGgugggcg, CACgugagga, AAGgugguuc, UGGgcauucu, AUGguaagcc, AGGgucagug, AGAguacgua, AAGguaggca, AAGguauuca, CAGguagauu, GAGguauuua, GAGgucuaca, GUUguagguc, CAGguacucg, GUCguauguu, AAGguacuuu, AGAgugagau, AGUguuggua, AAUgugagug, AAGguagauu, AUGguuugua, GAGgccccag, AUGgucaguu, UCUguaagga, CAGgucgggc, CAGguaagcc, UAGgucagug, AGAguaggaa, CUGguacuuc, CUCguaagca, CAGguaacua, CAGguggcug, UGGguccgua, GAGguugugc, CAGgugcgcg, AAAguauggc, UGAguacgua, CUGguacgga, CAAgugaccu, AAGgugaugu, AAGgucugca, AAAguuugua, AAGgugagca, GAUguaagcc, CAAguaauuu, CAGgugugug, UGGgugaggg, AAGgugaccu, UAGgugugag, CAGgcagguc, UCAguaaguu, UCAgcaguga, AAGguaccac, UAAguaggug, AAGgucagcc, CAGguaacuc, AAAguaagag, AAGguagaua, AAGgcaaggg, CAGgugucgg, CAGguggcua, GAGguugcca, CAGgccgugg, UUGguauaug, GAGguugagu, GAGguagguc, GUGguaagac, UAGguccuuc, GAGgcaaguc, GAGguaacau, CAGguauauc, UCGguugguu, CAGgugaacc, CAGgucuuuu, CAGgcauggc, AAAguacuug, CAGgugauuc, UUGguagguu, UAUgugagca, CAGgugagcg, AAUguaauaa, AAAguaaggc, UAGguuuguc, UAGgugggag, GAGguaaguu, AAGguagccg, CAGguggugc, UGAgucaguu, CUGguaggcc, CAAguaagga, CGGguaaggc, AAGgcgagga, CAGguaguuc, CAGguaagga, CCUgugagug, AAGguaaaug, CCGguaauua, CAGguaaguu, AAGgugguca, CAGguaccuc, AUCguaagua, CCGguacaua, GCGgugagug, GAGgugguau, CUGgugugga, GAGguaauuc, CAAguacgua, UCUguaagug, AAUguaagug, AGGgucuguu, GAGguacugc, AGGguaaggc, AAGgcaagag, CAGguggguu, UAGguuagga, UGAguaagcu, AGAguaagag, AUGgcaggug, UAGgcaagua, AUGguaggua, GCAgcccgca, ACGguaaacu, AGGgugaguu, GUAguagucu, GUGgcugaaa, CAGguuaguc, CUGgugagca, UCAguaagug, AAAgugauug, UAGgucugga, GAGguguuuc, AAGguaaauu, CAUguacauc, AAGguuugaa, CCAgcaagug, UAGguaauaa, GAGgcaagug, CAAgugauuc, CAGgucgugg, GAAguaugcc, UCGgugcccu, GAGgucaguc, CAGgugagac, UUUgucugua, CAGguagaua, UGGguaucag, UAGgugggcu, AUGgugagau, CAGguaacac, CCGguauccu, UAGguaagcu, UCAguacauc, UAGguuugcc, AUGguaagaa, UUGguaagac, CCGguuaguc, GAGguaagaa, UGGguaaguu, CCGgugagaa, CCUgugaggg, ACGguaggag, ACAguauguc, CAGguauuaa, CAGguggauc, AGAgugcgua, AAGgugaccg, AGAguaggug, ACUguaugua, UAGgucaauu, AGUguguaag, CGGguaccuu, CUAgugaguu, CUAguaagug, CAGguacaac, UAGgugugug, CAUguacggc, AUGgugugag, AGGguggaag, CAGgugcgag, UAGgugcucc, AAGguggugg, AAGgucuguu, CAGgugggcc, AAGgucaguc, CAGguuuuua, AACgugaggu, CGGguaagag, UUUgucggua, UAGguuaagu, GUGguaagaa, CAGguauugg, GCUguaaguu, CUAguaagua, UCGguaaaua, CAGguaacuu, CCUgugagua, CAGguuauau, CUGgugaaca, AAGguauaaa, GAGguaagca, AAGgugaagc, CAGgugaguu, UUUgugagua, CUUguacgcc, AGAguaagug, UGGguaggug, UGAgcccugc, UGUguaugua, AAGguagagg, GAGguggggg, UAGguaauuc, AAGgcauggu, AGAguaagca, AAGguaggaa, CAAguaagua, ACUguaauug, CAGgucugug, UCGguaccga, CUGgugagag, AAGguuugcu, AUGguaccac, UAAguuaguu, CAGguaggac, AGAgugaggc, CGAgucagua, CAGgucugag, GAGguggugg, ACGguauugg, GCUgcgagua, CUGguaagug, GUGgugagau, GGGguuugau, UCUgugagug, CUUgucagua, GAGguaaaac, UCUguaagau, CCAguaaguu, CAGguaaagu, GCGgugagca, UAAguaagag, CUGgcaggug, GAGguaaggg, UGAguaaguu, GAGgugagac, GCUgucuguu, AAGguaacaa, GAGguaacgg, CUGguauucu, CAAguaacug, AAGguggggu, UAGguauggc, CAGguauuuu, GUGguaaacu, GAGgucugag, CUGguaaggu, CAAguaaguu, AAGguagacc, GAGgcgagcg, CUGguaaaua, UGUguaagcg, CAGguuaggg, GGGgugagga, ACAguaugug, CCGgugggga, GAGgucagug, AGGguaaggu, ACAguaagua, GGUguaaggu, GAGguaauaa, CAGguauucc, CUGguauaaa, CCGgucugug, CAGguaacug, GCAguaagua, AAGguagggg, CAAguccacc, CAAguuggug, CAGgugcggu, CAGguaaaau, ACGguaagga, UGGguaauaa, UAGguaagug, CCGguagguu, AGAguaugga, CUCgugaguc, AAAgccggug, UUGguaauuu, GAGguaaaag, CCUgugugag, AAAguaagga, UGAgugagug, AAGguacaug, CCGguaaaug, CAGgugaagc, CAGguacccg, GAGguaaggc, UUUguauguu, CAGgugcucc, UCGguagguc, CGGgugaggc, AAGguaauua, ACUgugaguc, AAGgucagca, GUGgugagug, CAUguccacc, AAGgugaccc, CGGguuagua, GCGguaguaa, GCUguaggua, CCUguugagu, UAGgucuggc, GAUgugagcc, CUUgugagua, CUGguguguu, GAGgcaugug, CAGgcaagag, UUGguaagaa, GAGguguggg, GAGguauuuu, CAGguaguaa, AGGguaagac, UUUguaggca, AGGgugagau, GAGguuugua, AAGgugagug, GAGgugggag, AAGgugagaa, CUGguaagag, AUAguaaaga, GAUgugaguc, AAGgugcagg, CAGgucuguc, GAGgugauuu, CAGguuggcu, CGGguauggg, AUGguccauc, CCGguuggug, GGAguaaguc, AAUguaagga, CAGguuuguu, UAGgugugua, UAUgucuuug, ACGguacuuc, AAGgcacgcg, CUGguaaacc, CUUgugggua, UGAguaaguc, CUGgugggug, GAGguggaga, GUGguggcug, GUGguaagug, AACgugagua, GAAgcuguaa, CGGguaucuu, CAGgugucag, AAUguacgca, CCGgugggua, UGGgugaggu, AAGguauguu, CAGguauguu, CAGguuugcu, UUGguaaguu, CAGguaguug, CCUgugaaua, GCUgugugug, CAAguaauuc, AGGguaaugu, GCUgugaguc, ACCguaaguu, CGUguaagua, GGGguaaguc, AAUguaugau, AAUgugauua, UCAguaagaa, CAGguccguc, GAAguauuga, UUGguaagga, CAGgucgguu, UAGguuagug, ACGguaaaac, AAGguagguc, UACgugagua, UUGguaagca, GCGgugaguc, GAAguaaggg, CGCgugaguu, CAGguacccc, UCUguaagac, GAGgugggca, AAUguaagac, CAGgcaaggg, CAAguaacua, AAAguuuguc, CAGguacugu, AAGgucccuc, UCGguaaguc, UGGgugagug, CUUgugagau, AGAgugagcu, UAAgugggga, UAGguaggga, CAGguuagcc, AGGguaauca, AAGguucagc, UGGgugggug, CAGguuguga, AAGguaagug, CAUgugcgua, CCGguauauu, ACCguaugug, CAGguauagu, CAGguauuac, CAGgugcagg, GUGgugagcu, AAGguaacau, CUGgugaugg, AUGguaaaug, CCGgugagca, AAGguaaacc, AAGguacugg, GCGgucagga, CUGgucaggg, AAAguacguu, AGAguagguu, AGGguaagcu, AUUgugagua, CCGgccacca, GAGguaacuu, GAGguaugaa, CAGgucagac, UAGgcgugug, AGGguaaguu, CAGgcaugag, CAGguaacgu, CAGgcgagca, UAGguauggu, AGAguaggau, CUGguuucaa, GAGguaaacu, CAGgcaugca, UUGguaaucu, AGGgcagaau, AUGguaaaac, GCUgcaggug, GAAgcacgug, CAUguaaaca, UGGguaagau, AGGguagcua, AGGguggggu, CCUguaaguu, UGAgugaguu, GGAguaugua, CAGgugaccu, AAAguacgga, GAGguacaga, GAUguaggua, GGGguaauug, UAGguggguu, GUGguacgua, AAGguacagc, GAGgugaaga, GGGguaagca, UGAguagguc, GGGguaaguu, AUUgugaguu, UCAguaagac, AGUgugagcu, AAGgcaaaac, CUGgugaguc, AAGgucucug, GAGgcugugc, AGAgugagac, GAGgugaugu, AGAguauggu, UGGguggguc, GCUgcugagc, CAGguagcug, UAGgucagaa, CCGguaggug, GCAguaugau, CAGguuucag, GAGguuugcc, GGGguggggg, AAGguacaua, UGGguguguu, AGAguaaggc, GCGguuagug, AAGgugacuu, AUGguaagau, AUGguaguug, CAUguaagac, CUGguaugua, UUCguaagga, GAAguaugac, CGGguaauuc, UGGguaacuu, CAGgugccua, CAUguagggc, ACCgucagga, CGUguucgau, GAGgcaggac, UAGguaauau, UCGguauacu, UAGguugugc, CCGgugaguc, CAGgugccaa, CAGgugaugc, AAGgugagga, GUGgugaggg, UGGgucagua, GAGgucaggg, UAGguacgua, GAGgcaagag, CCUguuggua, GAGguaucca, UAAguaagcu, AAGgucaguu, AAAguuaaag, GAGgugcuau, ACGguaaguu, CUGgugaggg, GAGguuaugu, CUUgugugca, UGAgcugggg, AAGguauagu, UAGguaaaac, GGGgugaggu, GAGgcaagca, GGAguaacgu, AGAguaagua, AAAguaagua, GAGgcaacca, UGUguaaguu, UAGgugaggc, ACAguaagaa, UGAguaagug, CAAgucagua, AGGguaaaug, AAGguaugca, GCUgugcgug, GAGguucgcc, AAGgcuugca, CAGgcaagug, AUAguaaguc, UUGguaggua, GCAgcaggua, AAGguauauc, AGCguaagcc, CUGguucgaa, ACGgugggug, CUGgucauug, CAGgucagga, CAAgugagac, GAGguacugg, GAGguguagu, GAGguguccu, CAGgugcgua, AGUgcccuga, AUGgugaguc, UGUgugugua, CAGguaugcu, CUGguacagu, UUGguacgua, UCUguacgua, UAAguaauuc, CACguaugug, CAGgcaagua, UCGgugagug, GGUgugaguc, UCUguaagcu, AAGguucaga, AGGguacuuc, GCGgcagguu, GAGgcccgug, CAGguauaaa, AUGgucaagu, AAGgugagua, GUGguuuguu, AGAgugagga, GAGguaugac, UAGgcgugag, AAGguacucc, UGAgugagga, GAGguaugau, GGGgucggua, ACGguaugca, CAGguaccac, UAAguaccug, AGGgugggcu, CUGgucuguu, UAGgucagag, AAGguguguu, CUGgucagug, AAGgugggac, GUGguaguag, CUAguuuagg, CCCgccccau, GCUguacugc, GAGguaauau, UAGguuggug, AAGguccaac, UAGgugagga, GUGguaaguu, AGUgugagag, AAUguacaug, UUGgcaggug, UAGguuauug, CAGguacuga, GCGguggguc, UGUguaagau, GAGgugagua, GCAgccccgg, CAGgugcuaa, AGUguaagag, CAGguacauc, CAGgugggac, AGGguaaaua, UAAguaauua, CAGguaaccg, AAGguuugca, UAGgugguuu, CAGgugaccg, UGUguaagcu, GGAgugaguc, AGGguaggag, AGGgugggug, AAGgucugag, GAUguaauau, GGGguaauua, UAGguaggua, GAGgcaagua, GAGguaagga, UAGguacuac, UCGgugggug, AAGgugugga, CAGgucugcc, UAAgugagcc, GAAguaaguu, GAAguaagcc, UAGgugcgac, GAGguauggc, GCAguaagaa, CAGgugugga, UUGguaacgu, GCUguaaaaa, UUGguuagua, AUAguaaggg, UUGguacuag, CGGgcagccg, CAGgugcugg, UAUgugaguu, CAGgucuggg, UAAguaagaa, AAGguuauua, AGAguaaagc, AGAgugugag, UAGgugcgag, CAAguaaacg, AAGguacgua, CUGgugagua, CCAguaugua, UUGgugagug, UGAguaagua, GAGguuagca, GUGguaagcc, CUGguauggc, AAAguaacac, CAGguacuaa, UCUguaaguu, GAGgugaggg, ACUgugggua, GAUguuugug, CAGgugucaa, CAGgucacca, CCGgugagua, UUGguaaaua, CAGguggggg, ACUgcaggug, UAGguauguu, GGAgcaagug, UCGgugccuc, CAAguaacuu, GAGguaacca, CAGguaauau, GGAguaagaa, GAGguaccuu, AGGguaagga, CCUgugaguc, GAGguaaugg, AUGguguguc, GGGgugagua, AGGgucaggu, UGGguaaggg, AGGguagguu, AUAgugaguu, CCCguaggcu, ACAguaugua, GACgugugua, GCGgugagga, CAGgugaccc, UAAguuuagu, ACAguugagu, CGGgugaggg, CAGguggauu, CGGguagagg, UAGgugcgug, GGGguaagaa, GAGguggggu, CACguggguu, ACGguaauug, AGAgugaguc, UUGgcuccaa, AAGgugaugc, AAGguugguc, AGCguaaguu, AUUguaugua, UCAguuaagu, CAAguacgug, CAGgugcgug, CAGguaggua, AUGguggggu, AUGgugaguu, CAGguaauca, AAGguagggu, CAGgccaagg, GUGgugagag, AAGguuggug, CAGguacucu, UAGgcaugug, UUGguaccuu, CUGgugugcc, ACAguugcca, UUGguaauau, GAGgugcaug, UUGguuugua, UUGguaagug, UGUgugugug, GUGguuugua, GCGguacaca, AGAguaugcu, UUUguaagua, UCUgugcggg, AAGgucagug, GAGguaggaa, GCGguuagca, AGGgugaggg, GAAgugagua, CAGgugacag, AAGgugauua, GAGgccagcc, GAGgucuccu, UAGguauuac, CAUguaagag, CUGguagggc, GAAguaagua, CGGguaagug, CAGguaaucu, GUGguaggua, CAGgugggua, AAGgccagug, AAAgugaauc, ACGguuacgu, AUGguaggaa, CGGgugagac, GAGguuggaa, UGGgugagcc, CCAgugagua, CUAguacgag, CAGguaugac, GCUgugaggu, CUGguaugaa, GGUguacgac, CUUgugagug, GUGgugagca, CUGguaacuu, CAGguacuau, AGGguaaggg, UUGguuaguu, GGUguaagca, UCGgugagga, UGGguaaaca, UCGguacgug, UAGguagcag, CUGguaaggc, GUGguaagga, UAAguaagca, GAGguuccaa, CUGguaugga, GGGgugggua, CAGguuuccc, CAGgucucug, GAGgugagga, CUUguggguu, AUGgugagac, CAGgugaagg, GCGguagggg, GUUguuuccc, AAAgcaucca, GUGguagguu, AAGgugugaa, CAGguacagu, AAGguaccaa, UUGguaauug, AAGgugcuca, AAGguucaac, CAGguuuaca, GCUguaagug, AGGguauguc, GAGgucgggg, AAGgugccug, AAGguaaaaa, GUGgugaguu, UAGguaagaa, AGGguauccu, GUGguaauau, UCUguaagua, UGGguaugga, AUGguaugga, GACgugagcc, CUGguuuggc, AUGguauauc, AAAguaaacu, AGCgugagug, CUGguauaga, CAGgugggga, AGAguauguu, UAGguacuug, GCAguaggug, AGUguauguc, AAGguuaagc, CUGguggccu, GAAgugaguc, UUGguguaag, CAGguaagaa, CGGgucucgg, GAGgugcaca, CUCguuaguu, AAGgugauca, UAUguaagaa, GAGgugcuug, CAGgugguca, ACGguaaguc, ACAguaaugu, CCUguaaggu, GAGguuaagu, UCGguaugug, UGGguauguu, AAGguauuac, CAGgugaggg, UUGguaaaca, AAGguagugu, GAGguguggc, CAGguacgga, AAGgucauca, CAAguaggca, CAGgugaaac, CAGguacugc, AAUgcaagug, CAUguaauuc, AAGguaugcu, CUGgugaguu, CAGgugguuu, UGUgugagua, AAGgucggug, AUGguaaauu, AGGguauuac, AGUguaugga, AACguaagau, GUGguaaggu, ACUguuagua, CAGguaucag, AAGguuaguu, CUGgugagcu, UUGgugagcu, UGUguacgua, GAGgucagcc, GAGguagaau, AAGguaugag, UAGguauuuc, UGUguaacac, AGUguaaggc, GAGgucugcu, AAGguuagca, CAGguaaaug, AACguaagcu, CAGgucugca, CAGguauugu, GUGguaauuc, GAGguauaug, GCCgugagcc, GAGguaagag, UGAguaugua, CAGguaaggg, GAGguaaauu, CAGgcaacuu, UGUguaaguc, CAGgugcgcu, CGGguaaacc, CCGgucaguc, UAGgugggcg, GCGgucaguu, GGGguggguc, AGCguaauag, ACGgugaguc, CUGguacuug, CAGguuggua, AGAguaugug, CUGgugggua, GAGguggcuu, AUAguauuga, UGAgucgucc, CAGgugcucu, UACguaauau, GCUguccuga, CAGgcugcac, CUGgugcgcu, GCGguaagaa, UAAguuacuu, GAAgugagug, UAGgcaaguc, UAAguaaaua, ACGgugagug, CAGguagguu, GGGguauaac, GUUgugaguu, CAUgugagua, GAGgugcauu, AAGguuugua, UCGguaaugu, CGAguaaggg, GAGgcacgga, AGGgugugga, CAGguauggu, AAGguagaaa, CAGgugccug, UGGguauaug, UGAgugagac, UGGguaauuu, AUGguaaaua, AAGgcaaagg, AGUguuuguu, AUGguauugg, CUGgugaggc, UUGguaaaau, ACAgugaguu, CAGgugcugu, GAGguuaaga, AGAguaagaa, GAGguccgcg, GUGgugagga, CAGgugagcc, CAGgugacau, AUGgcaagcu, UCGguaauau, CAGgcaacaa, GGGguaggga, CUGgucucgc, UAGguaacga, CGGguaaggu, UAGguaaugc, CAGgcaagaa, ACAguaggua, CAAguaugag, GCUguucgaa, AAGguuaugc, GAUgugaguu, CAGguggaga, AGAguuaguu, UGAgugugcg, GAGguacagc, CAGguaagac, CAUgugcuuu, AGGguguguu, ACAguuaagg, ACAgugaggg, GAUguauacc, UUAguaagcu, CAGguaagau, AGAgcugcgu, GAGgcaaguu, GAAguaagug, AAGgugaaaa, AAGguaccua, GAGguaucag, AUGguaugua, AAGguaugaa, UUGgugagcc, AAGguuagga, AGGguaugua, CAGguaccga, AGAguaaacu, AAGgugcaua, AAGguaaugu, CCGgugugug, AGGguaaauu, GGGguuuggc, CAGguacacg, UUGguaacca, GAGgucaggu, UCUguuggua, CAGguuaguu, UUGguauguc, AAGgugcguc, AGGguaagaa, UUUguaagcc, AAGgucaggu, CUGguaaacu, UCGguaauuu, CUGguaggcu, GAGgucugua, GAGguacuuu, CUGguaaagg, CGGgugugug, CAGguguggu, UCGguacguc, CAGgugccag, GGGgugagaa, ACAgcuagua, AAGguauagc, CUGguaggag, GCUguacgua, AAGguaaagg, CAAgcacgag, CUAguaagac, CCCguaagcg, CAAgugugag, AUGguaaggg, AAGgugaggg, CAAguaggua, GGUguugcug, GAGguacugu, UAGguaagau, CAGgugcgaa, GAGguccagg, UUGguauaca, GGAgugagua, GAGgugagau, AAGguggggc, CAGguaaacg, UCGguaacuu, CAGguaaauu, GAGgugcgca, ACUgugagua, ACGgugugac, GUGguaaguc, CAGguaggca, CAGgucagca, GUGguaugug, AAAguaucug, CGGguaugua, AAGguaauaa, GAGgugggga, GCUguaggug, GAAgugaguu, AAAguauuua, UAUguaagua, ACGguaugag, CUGgugagug, AGAguaaaau, GCUguauggc, AUGguaaacc, GCAguaauaa, UAAguauuua, AAUgucagug, AUUgcaggag, CCGguaagaa, AAGgcaaguu, GAGguuuguc, AAGguaacug, AAAguaugag, GAUguuagua, CAGguggguc, AAGguaccga, CCAguaauua, GUGguaugcg, AUGgugcgcu, CAGgucuaug, AAGguauuua, CUAguaagau, AGAguaauuu, GAGguaacgu, AAGguagcca, CUGgucccgg, GAGguccuuc, ACGgucaccc, AAGguaauac, CAGgugcaug, AUGguaauag, UUUguaacac, UGGguaugau, CAGgcccccc, AGAguaguaa, AGUguaagaa, GAAguauguu, CAGgugugca, UUGgugaggg, UGGguugguu, CAGguacgua, GAGgugcggc, UCUguacggg, CGGgugcgug, UACguaagug, CAUguaagga, CAGgugacgg, GAUguaugcu, UCUgcaauuc, UGAguaaggc, GAGguauauu, AGAgugaguu, AAGguaagcu, UAGgugaagu, CAGguuagua, UAUguaagug, UUGguggggg, UGAgcucaaa, UCGguaugua, UAAguaugcc, AAUguaagua, CAGguuugca, ACGgugagag, CAGguguuuu, GUGgugagcc, AGGguacaua, UAGguaaccc, GUGgucagua, CUGgugagcc, CAGgugcuua, AUAgucguga, AUAgugagug, GAGgucaaaa, CGUguagcuu, CAGguguuug, CAGguuggac, CAGguaagcu, AGGgucagaa, CACguauguc, CACgugagug, GGGguacgga, AAGgcaggac, GAGgugaagc, GAGguuugaa, CAGguaagug, CAGguaacca, CAGguacucc, AAGgugcuuu, GAGguaaaua, GAGgcaggug, GAGguucgga, CAGguauuug, CAGguaaaua, CAGgugaugu, CAGgugauac, GAGgugaggc, AGGguggggg, UAAguaaguu, UGGgugaaca, UAGguacugc, CAGgcuccug, AGGguaggca, CAGgugcccg, GAGguacauc, AGGgugugug, AAGguaguaa, UGGguaugag, GGGgugugug, CUAguaggug, GAGgcaagga, AAGgcaagac, AAAgugcggu, AAGguugguu, GAGguuaaug, UUGgugaguc, UCGguuagcu, GCAguaagca, AAGgcaagca, ACAguaagcu, GAGguaacag, AAAguacgua, GAGguaauac, UUGguaggug, CUGguuaguc, GAGgugacgc, ACAguaagga, AAUguacuua, GGGguacagu, CGUguaugug, UCCguagguu, GAGguggucg, UCAgugaguc, AAAguaagca, GAGgucuggu, GAGguaauua, GUAguaagua, AAGgugggga, UCUgugagca, GAAguucgug, ACGgugaggc, UCAgugagua, UAGguaguug, GGUgucuggg, GGGguaagug, GAGguggguu, UGUgugaguu, CAUguaagua, AAGguaggug, AAUguaggag, GAGgcacguc, CAAguacauu, UUGguacaga, GAGguaguag, AAAgugaggg, UUGgucagug, AGGgugaguc, CAGgugaaca, GGUgugggcc, CGGgugagcu, GGGgugaguc, ACAgugagag, AGGgugaggu, GCUguaaguc, AUAguagguu, CAGgcaugug, AAGguaaguu, CAGguccgug, GAGgcaggua, AUGguggaag, AUGgugggcg, GAGgugagaa, AGUgugagca, UUGguaagua, CAAguaagca, GGUgugagcu, CCCgugggua, CAGguagaau, CAGgcugagc, CUGguggccc, UGAguaagag, CACguuagcu, AAGgugaguc, AAGguagcuc, UCGgugaguu, GAGgcccuuc, CAGguuaugc, CCUguaagcu, CAGgucuccu, UAGguaggcu, GGGguagggg, AAGguaguga, GAGguuguug, CAGguugguu, AAAguaagcc, ACAgugagug, UGGgugugau, CCCguaacua, AAGguguugc, AAAgcuggug, GAGguauagu, ACGguaagag, AUGguacggu, GAGgccaguu, GAGguaugcg, UCGgugggag, AAGguggaua, CCAguguggc, AGGguaagug, UCUguagguc, CAGgcaagga, CGGguaauuu, AUUgugaguc, CAGguaaacc, AAGgucaauu, AAGgugaaua, GUCguaagaa, GCGguaaguc, CUGguagagc, GAGgucgguc, CAGguaaaca, AAGgcaagga, CAGgucgucu, GGGguagggc, CUGguacuaa, GAGguagcug, CUUgucagcu, UAGguaaggc, CUGguauuac, UAAguacguc, AAGguaagcc, ACGgugaaag, CCAgccaaua, CAGguuuguc, AAGguauaau, AAGgucuuag, AGGgugagcu, AAGguuaggg, CGGguaaauu, CAGguaacgg, AGAgugugua, ACAguaaguu, GAUguaauuu, GAGguaggga, UUGgcaagug, AAAgugagga, AAGguagugc, AGAguaauuc, GGAguaaaua, GUGguaccca, CAGguauugc, GAUgugaggg, CAAguaaauc, CAGgugucuc, AAGguaacag, UUGguaaaag, CAGguaucau, ACGgugagac, CUGguaugac, CAGguucacu, GAGgugauca, AGUguaaguc, AACguaagua, AAAgugagug, GAGguacagg, CAAguaauga, GAUguaagga, UCAguucccc, GCGguaagga, UAGguacuaa, AAGgugaaag, ACUguaagug, UGGguaugug, AUGguaacag, CAGguagggu, ACAguaagug, AAGgugcucc, AAGgugugcu, AAGgugguga, ACGgugcgcc, AAGguauugc, GGGguaugug, CAGgugggcu, GAGguauguu, AACgugaaua, CAGguaaugg, UAGguaugau, CAGgcaggug, GGGguugguc, AAGguauggg, UAAgugaggc, CAAgugaucg, AAAguacggg, AGAgcuacag, GAGgugggaa, CAGguacuuu, GAGgugagag, CAGguagguc, UGGguacagc, AAGgugucag, AAGgcaagaa, GAGguaaaca, AAGguaaagu, AAGguaguca, CUGguauguc, GAGguauggg, AAGguauugu, CUGguacuga, GAGguaagcu, UGGgugggua, CAGguucgug, AAGguauggu, CAGgugagca, UGGguaaauu, UGUguaggug, UGUgugagcc, CUGguaauau, AAAguauguu, UGUguaagaa, CUAgugagaa, AGGguagguc, AAGgugggug, UCGguaagug, AGUguaaaua, GAUguaagug, AAGguuagug, UAGguaagca, CAAgugagaa, AGUguaagua, CAGgugaauc, UGGgugagac, AAGguagggc, CUGguuugug, GCGguagggc, GAGguaaucc, AUUguaauaa, CUGgugaaua, AAGguuuaaa, CCUguacugu, GCGgugagcg, AAGguaaucc, UAUgugagua, CCCgugagug, CAGgugcaga, CAGgucaguu, CAGguaggcu, AAAguaagug, UAGguugguc, CAGguugccu, AAGguaugga, GGUguggacg, AAAgugagaa, AGGgugagag, GAUguggcau, UCGguaaggu, GAGgugcguc, CGGgugaguc, AAGguacggg, GAGguucuug, AAGgugcuug, UAGguaugua, AUGgucagca, CGGguacuca, AGGgugagga, AUCgugagua, UCAguaagua, UAGguaaaua, AAGguaauug, GAAgucagug, CAGguacaaa, AAAguuaauc, AGCgugagcg, CCGgcuggug, AGUguaauuu, UGAgccacuc, GGGgucugua, AUGgcauguc, CGGguaaaga, AGGguagcau, CGGguaggag, GAGguucgug, UAAguuauuc, UAUguaagau, AAGguaguuu, CAGgugguau, GUGguaauga, AAGgugauuu, CAGgugaagu, GUAguaauua, AUGguuggug, CCAguaagug, UAGgugagag, AUGgugaggc, AAAguuagug, AAGgugccuu, UAGguaugag, CAGgugugac, CUGguggguu, AUGguaagga, UCUguaagaa, UCCgugaguu, AAAgcaggua, UAUgugagug, CAGguggagg, CAGguuagac, AUAguaagac, AAGguguugu, GAGgucugug, AAGguaagau, CAUguaaguu, CUGguaauua, CAGguaggcg, AGAguaaguc, UGGgugagga, AAUguaggua, UAGguuagca, GGGguaggua, GAGguauugc, AUUguacaca, GAAguaggua, GGAguaagcu, UAGguaugug, GAGgugaaua, GAGgugggau, AAGguaaucu, GGUgugaguu, AACgugaguu, GAGguaaccg, UAGguaagga, AUUguaagaa, UGGgugagca, AAGguaaggc, CCAguaucgu, CCGgugggug, GAGguagugu, ACGgugggaa, GAGgugaccu, CACguaugua, AGGgugggga, AAUguaaguc, AAAguuaagu, CAUgugagug, AGAguauguc, GCGguaugac, CGGgugaguu, CCGguauuuu, GAGguagaac, UAGguaugaa, CAGgcgcgug, CAAguaaguc, AGUguaagau, AAGguucuac, CCAguaagua, GAGguagcag, CAGgucuguu, CAGguacaau, CCGguaaaga, UAAgugcugu, AGGgugagaa, CUCguaaggu, CAGgucagcu, CAGguaaggc, AGGgugcagg, GAGgugaaac, AGGguaagua, AAUguaugcc, AAGguaagca, ACGguacggu, AAGguaauga, UCUgcucaau, ACGguaaugu, AAGguaguug, ACGguaagug, CAGgugauga, GAGguaacac, GAGguaggua, CAGguaccuu, CAGguaauaa, UUGgugggug, CUGguaauga, UAGguaaguc, AGGgugugac, GAGgcaauaa, GUGguaaagc, CUGgugggcg, GAUguauguu, AGGgugagac, UCGgucagca, AUGgugauua, CGAgugugua, CAGguuggug, AGCgcaagua, UGGguacguu, GAGguauuug, AGUguacaua, AUGguaagua, ACAguagguu, AAGgugagag, UUGgugaagu, AAAguaugua, UGGguaagga, UAGgugccuu, and CCUgugggug.

Additional exemplary gene sequences and splice site sequences (e.g., 5′ splice site sequences) include UCCguaaguu, GUGguaaacg, CGGgugcggu, CAUguacuuc, AGAguaaagg, CGCgugagua, AGAgugggca, AGAguaagcc, AGAguaaaca, GUGguuauga, AGGguaauaa, UGAguaagac, AGAguuuguu, CGGgucugca, CAGguaaguc, AAGguagaau, CAGgucccuc, AGAguaaugg, GAGgucuaag, AGAguagagu, AUGgucagua, GAGgccuggg, AAGguguggc, AGAgugaucu, AAGguaucca, UUCguaagua, UAAgugggug, GCCgugaacg, GAGguugugg, UAUguaugca, UGUguaacaa, AGGguauuag, UGAguauauc, AGAguuugug, GAGgucgcug, GAGgucaucg, ACGguaaagc, UGAguacuug, CGAgucgccg, CUGguacguc, AGGguauugc, GAAgugaaug, CAGaugaguc, UGGguauugg, UGAguaaaga, GUGguuccug, UGAgcaagua, UAUguaagag, AAGgucuugc, AAAgcaugug, AGAguacagu, GUGguaaucc, CAGguagagg, AAGguacaac, UGGgcagcau, CCGgucauca, CCGguuugua, UGAguaaggg, GAAguaugua, GGGguagcuc, GCUguacaua, CUGgucucuu, GUGguaaaug, AUCguaagug, GAGgcaugua, AAGgucuccc, UGGgugcguu, UGUguagguu, GAAgugagca, GGUguaauuu, CUGgugaaau, AUCguaaguc, AGAguaaucc, GGAguagguc, GAGguaccaa, CUUguaggug, AAGguauaag, AGAguuggua, AUGguuugug, UGGgucagau, AGAguaggac, AGAguagugu, AGAguaggag, CAGgucucua, AAGguggaug, UGGguaucaa, GAUguaugga, AAGguguuuc, GCAguguaaa, UUAguaugua, UCUguaugca, AAUguaaaau, AGAguaaauu, GGGguacuuu, GAAguuugau, AAAguagauu, UGUguagagu, UGGguaagcg, CGGguucagg, AGGguacgac, UCGguaagaa, AGGguuggca, AAAguacagu, UAAguuaagg, AUGguaaugu, GUGguuuuac, AGAguaacaa, AAGguagccc, GCGgugaggc, AUGguucagc, AAGguacuua, AAGguccgug, UAGguaagcg, AUGguaccuu, GCCguggugg, CUGgugcguc, CAGguggaaa, AAAgucugua, GAGguaaccc, AGAguauggg, UAUgccccug, AAGgugccag, ACGgugcggc, AGGguacuga, AGAguaagcg, CUGgcaaggg, CCAgugugug, GAGguagacg, CGGgugcggg, GAUguaagcu, AUUguauuua, UGCgugagug, CUGgucuaua, GAGgugcuag, GAGgugccau, CAGguacguc, GAGguucagc, AACguaagaa, AGAguaguac, AAGguaacgg, UAGgugugac, CCGguaauag, CAGguaccag, UUUguaauug, AAUguacgaa, CAGguaauga, AUCgucaagg, CUGguagaug, GGGgugcagu, AGUgugagaa, GGGguuuuau, CCUguccccu, AUUgugaagu, AAGguaaacg, UACgucgugg, AAGgugccau, GGGgucccag, UAUguauggu, CGGguaauua, CGGguacucc, CAGgugacuu, AGUguggguu, AGAguauggc, AAGgccaaca, AAAgcaagua, UCAguagguc, GUGguggcgg, CAUguauccu, UCGgugagcc, AUAguugggu, AAUguuagcu, AUGgugaaug, CGGguaaugu, UCUguaggug, CCGgugaggc, UGAguccacu, CUAguaagag, CGGguggggc, CGAguaagca, UGUgccaauu, UCGguaagcc, UAUguaggug, UUGgugggcc, GAGgcugggc, AGAguaacuu, ACGguagguc, CAGgcccaga, CCGguggguu, AAGgugacgg, GGGguacagc, CAUguaaguc, AUUgugagaa, UGUguaagga, UUUguaagau, AGGgucauuu, UGGguuuguu, CGAguaagcc, GUGgugugua, AUGguauaac, UGGguacgua, AAAguagagu, UCGguaacug, AGAguaauga, AUGguggguc, AGAguaauau, CAGguacugg, UAAgucaguu, GCGguagaga, AAGgugaugg, ACAguauguu, GAUguacguc, UAGguuucuc, GAGgcauggg, AUAgcuaagu, GUAgucugua, AAGgugaacg, GUGguggucg, GAGguugauc, UGAguggguu, ACUguacgug, CUGgugacug, CAAguuaagc, GAGguaccca, AACguaacuu, CAGguuacua, AGAguuaguc, UGGgcacguc, AGUguauggu, AAGguugcaa, CAGguuguua, AAGgcauccc, GAUguaaggc, AGGguacggg, GAGgucaaag, CAAgugagcg, AGAguaaucu, UCGguagcug, AAAguaguag, CAGguucguc, CGUguaugaa, AGUguaaaaa, AAGgucucac, UAGguggagc, UGAguaggug, AGAguaugcc, GAGguugcau, CAAguaagag, UCUgugugcc, GAGgugaugc, GGGgugauaa, CCCgugagcc, AGAguaacug, GCGguaagua, AGAguacauc, UCGgucuggg, UAAguaucuc, GGCguagguu, AGAguacgcc, GAUgucuucu, AGGgcaaggu, CGAguaugau, AUGguagagu, CAAguacgag, UCGguaugau, CCGguguguu, AGGgucugug, GGAguaggcu, AAGgucuaug, GCAgugcgug, UGGgugagaa, AGGguaaagu, GAGguaggac, CUAguaagca, UUAguaggcu, CUGgugggau, CUGguuagua, AAGguacgug, CGGgugagau, AAGgugcaug, AAUgugggcu, CAGguugacu, CAGguuacag, GCGguaacau, AUUgucaguc, CAAguauaca, GAUguccgcc, AAGgugcgga, AACguaagag, UGGguuggua, CAAguguaag, GUGguaacgu, CUGgugauca, AGGguggggc, UCGguaaaga, CAGguacacc, CGGguaaggg, CAAguuugcu, ACAgugcgug, UUGguauggg, GAGgcucauc, CUGguaauag, AUGguggaua, UCAgugaauu, AAUguaauua, GCAgucuaaa, AAGguauucu, GAGgucauca, UGGguccaug, AGAguuugua, AGGguagacu, AAGguaggac, UGUguguuga, UCAguacgug, AUGgucucuc, UGAguuagua, UGAguaaagu, GAGgugaccg, GAGguauauc, CAGgugccau, AGAgugguga, GUUguaagaa, AGAguaaaua, AGGgugaagg, CUGguagauu, GAGguucagg, AGGgucuuca, CUGguaaccu, ACAguacuga, AGAguggguc, AUGguaugag, AAGguuauau, AGAguauagu, AAAguaugaa, UAGguggcua, ACCguauggg, AAAguauaau, UUUguauggc, GGGgucgcgu, GUGgugguuu, CAGguuugac, GGAguaggcg, GAGguacccu, AUGgugugca, GUGguuggug, AAAguaugcu, UAAguuacau, ACAguaugag, GGAguauguu, UUUgugagaa, AAUgugcguu, CAGguagagu, AUGguguuaa, CAUgugcguc, AUAguuggau, GAGguacgua, GUUgugagaa, CAAguacauc, GAGguaguuu, ACUguacaga, CCGguuguga, UGGgucagug, GUAguaagaa, GACguacuuu, AGAgucaguc, UAGguuaguu, AGGgcagcag, AAGguccuac, AAUguaauug, CAGgugcggg, CUGguaaugg, CAAguagccc, GAAgucaguu, ACAguaauug, UUAguuagua, CCUguauuuu, AUCguaagaa, CCAgugagca, GAAguaaggc, UGAgugggua, UCAgugguag, UCUguacagg, CGAgugagug, UCCguaugug, CAUgccguuu, AAAgugacuu, AGAguaggca, GAAguaagag, CAGgcagguu, UUGguagagc, AAGguggaaa, GAGgcagguc, AUGguacgac, AGGguaggaa, AGGguaggua, UUGguaaggu, AUGguacaga, CAGguagagc, UAGguaaggu, GGGguuagag, AAGguaucaa, GAGguagccc, CAGgugccuc, GCAguaagag, ACGguagagu, UGGguaaugg, CUGgucaguu, GUGguacauu, AAAguagguu, AAGgccaaga, CGGgugggca, ACGguccggg, CGAguaugag, CUGguaugcc, GAGguggaug, CAGgccuuuc, AAAguacauc, AAAguaauca, GAGguaacug, CUGguaaaga, CGUguaagca, UGGgcaagua, GCGguggcga, GAGguggccg, AUUgcaugca, ACGgugacug, CAGgucagau, AGAguaacuc, UGAguaacag, AAGguacccg, AGGguaggcu, GGGgcaggac, CCUguaagug, AUUguaagug, ACUguacgag, GUAguagugu, AGAguaugag, UCAguguggg, UGGguauaua, UAGguagcua, GGGguaaaga, AGGguuacuu, CAUguaaaug, GGAguaguaa, CAGgucaauc, CGGguuagug, UAGguacaug, UAGguuaaga, UGGguaccuu, CGGguggaca, CAGgucuuac, AAGguggagc, AUGguaacca, UCGguaaguu, UAUguacaaa, AAUguagauu, GUAgcuagua, AAGguauugg, GAGgucuuug, GAAguucagg, UGGguaucac, AGAguacugg, CAGguuaaug, AGGguacgug, AGGgcacagg, CUGguuaguu, UUGguacgag, ACGgugauca, CCUgugagag, GAGgugaagu, AAGguacauc, UCUguaugug, UUGguggaag, UGGgcagguu, GAAguggagc, ACAguaagac, CGGguaccaa, CAAguacguc, AGAgugaggg, CGGguaagaa, AAUguaggug, AUCgugugcu, UAGgucaugg, CAGguuuuga, AAGgcaugca, GAGgugcugc, AAGguuaaua, CAGguucauc, GCGguaggug, GACgugagua, CAGgucuacu, UUGguaugag, AGCgugggca, AUGguaaggu, AUGguaccuc, UUGguauggu, UAUguaugaa, UGGguauggg, GAUguaaaua, CCGguaaguu, GAGgucugaa, GAGgugcgag, CUGgucagcc, CAGguuuugu, CGGguggugu, UAAguuagua, UUUgugugug, CAGguuaacc, UUGguacuuu, GCUguaaggc, AGGguggcug, GAUguaaaaa, AAGgucaaaa, CAGguagcgc, CAGguuuggc, GAGgugguuu, CGGguaaaua, CUGguucggu, GGAgugagcc, AAGgugcgcg, GAAguacauc, AGUgucugua, CCCgugagcu, GAGguucaca, CUAgugggua, GAGguaacua, UCGguauguc, UAAguauuug, CAGguaagcg, GAGgugguaa, CGAguaagag, CCGguaagcu, GAGgucuugu, AAGguggguc, CACguaagug, AGUguaauga, AAAgugugua, GGAgugccaa, CACgugaguu, AAGguuggau, UAUguaaaua, CUGguaggaa, UAUguaaacu, AAUguauuuu, CUGgcaagug, UGUgugguau, UAUguauguu, UUGgugacuc, GGAguaaggu, AAGguagaug, UGGguagggu, AAUguaauuc, GUGguauggc, GGAguggguu, AGGguaccac, UAGgugacag, ACAguaggca, AUGguuugaa, GCAguaacua, CCGguaggua, AGAguaggcc, AAGguugaca, CUGgugugua, GAAgucuguc, UGGgcucgga, CAGguagccu, AGAguaggua, UAAguauguc, CUGguauauc, GAGguguguu, AUGgugcaug, AAGguacgcc, UGAguaacua, GAGgugacag, GUUguccugu, UUGgugucuu, AAUgugaagg, UUGguggaua, UAGguguguu, CUGgcaaguu, GCAguaagau, GCGguggaaa, UGCguccagc, AAAguggagu, CGUgugagcc, AGAguacugu, CAGguauagc, UACguaagga, AAGgucuuua, AAGguggucu, GGGguaaauu, UCAgugagga, AGAguacguu, GAGgucguca, UAGguuugau, CAUguaaacc, AAGguggcac, CAGguagaug, AACguaaaag, UAGgucucug, AUAguaggug, UAGgcaagag, UAGgcacggc, AAGgucuuca, CCAguaugcu, CAAgugaguu, CAGgucucaa, CAGguuacau, GGAgugagca, AGAguacgca, CUGguguugg, AAGguacuca, CUAguaaggg, AGAguaaaag, AAGguaacga, CUGguccccg, UAAguauggg, GAGgucgagc, UUGguauaua, AAAgucaagg, AAGgucuagg, CGAguagguc, AGGguucguu, GAGgcaggcc, CUAguauuac, ACGguaugug, UAGgugguuc, AGAguauaac, UUGgugcguc, ACCguuaucu, CCAgugauga, GAAguaugca, GAAguauggc, CCGguaggac, AAUguaagca, AGAguaauug, AGGguugguu, GUGguaggag, AAGgcaguuu, CAAguaagcc, CUGgcaagua, CAGgcaugau, AGGguaauug, GGGguaaccu, AAAguaacua, UAGgucugcc, ACGguaugaa, AGUguauggg, UGGguuggca, UAGguaaacu, AGAgugggua, AGAguauuug, AGUguaggaa, CUUguacgua, GAUgugagau, CAGgcagcca, AAGgucacug, AAGgucugac, UAGguuccuu, CUGgugcuuu, UGAguuggug, UUGgugggau, UGAguagggu, UCGgugaggu, AAAguaaaga, AAGgcaaguc, CGGguaaagc, AAAguuaguu, UUAguaagca, GAGgucacau, UAAgugguau, UAGgugcuuu, GGAguaggca, UGAguaagga, CAGguggagc, GAUguagaag, AAUgccugcc, AUGguaaggc, UGGguaauau, CUGguaccuc, CACgugagcc, UGAguuugug, CCGguagugu, AAAgugacaa, GAAguggguu, CAGgugcagc, GAGgugggcc, UAUgugcguc, GGGguacugg, CUGguagguu, UUGgcauguu, AAUguaauac, UAGgccggug, AGAgucagua, UAAguaaauc, CAGguuccuc, UAGguacgau, AGAguuagug, GCAguaagug, AGGgugguag, GGAguaaugu, GAUguaaguc, CCAguuucgu, AAGguucggg, AUGguggagu, AAGguaccgg, GAAgugcgaa, UGGgucaguu, AAGguguaga, UGGguaggcc, CCAgugaguc, AAGgucacuu, AGCgugaggc, UCCgugguaa, AGAguacuua, GGGgucagau, AAGguggacc, AGAgugagcg, AGAgucagau, UAAguauuac, AGAguauuuc, AGAguucagc, AUGgugaagu, UAGgugaucc, GGAguaagau, UAGguaccaa, AGAguugguc, GAAgugagac, AUCguagguu, GAGguacgcu, ACGguaaggg, CAGgcauguc, UUAguaagau, UGAguagguu, AGGguacgaa, ACGguauguu, AGGguacugu, UUGguaugga, UAAguaacug, GCGgucagcc, UUUgugaguc, GUGgucagug, CUGgucugua, GAGguucuua, AUGguacuga, AAUgugcuuu, AGGguggcgu, CCGgcaggaa, CAUguggguc, UUGguuuguu, CAGguucugu, ACGguaagcg, CUGgucagua, UCAguaggcu, UGAguaggac, CAGguuuuaa, GAGguguccc, AGGguggguu, GUGgugagac, CACguaggga, GUGguauuuu, GAGauauccu, AAGgugaaca, UAAguagggc, CUGgugcggg, CUGgucaaua, AGAguaaaaa, AAGgugcagu, CGGguaagca, AAAgugagcc, AUGguaauca, GCAguacgug, AUGguacaug, AAGguuaaga, CGGguaaaug, GAGguucgca, GAGgcucugg, AUGgugggac, AACgugguag, AAGgugauag, GGGguuugca, CAUguaaggg, UCAguugagu, AAAgugcggc, AGAgugagcc, AUGgcaagaa, ACAguaaggu, AAGgucucua, GUGguaaaaa, AAAguaggug, UAGgugcacu, GUCgugguau, CAGguauagg, UGAgugagag, ACUgugagcc, AUCguuaguu, UUUguaccaa, UGGgugagau, AGAgugagaa, AGAguagggg, AGGgcaagua, CGGgucagua, UUGguaugcc, CGGguuagau, GGGgugaagu, CCCgugugaa, GCAguuugga, UGCguaagac, AGAgucugua, CACgugagca, AGGguaaaag, CAGgcugggu, GAAgucuuca, AAGgcaaaaa, GUAguaaaua, CUAgugagag, GAAguuucug, CCUguacgua, GAGgugcgcg, AAGguguaaa, CCAguauguu, CCGgucagcu, AUGguuccug, CAAguuaaau, AGAguaggcu, AUGgugggca, GGAguaagac, AGGgucacga, UAGgugauau, GAAguaaguc, CGGguaagau, CAAguagcua, UGAguaaaau, GUCguacgug, AUGguacgua, CAGgucucgg, GAGgcauguc, AGAgugggau, GUGguuagag, UGGgugguga, AAGguuaaac, CUUguuagcu, AAAguaggaa, UAGguuguau, AGGgugcgcc, AAGgugggcu, UAAguaucug, AAGguaacgu, AUGguggggc, CAAguacacg, GGCguaagug, AUAguaggac, AGAgugaggu, UUUguaaaaa, GAAguuugua, CUAguaaucu, AAGguuuuua, GAGgugcguu, UAGgcgagua, ACCgugagua, CAGgucccga, AUGguacugg, UGAguucagu, AAUguguggu, UCCguugguu, CAGgucagag, CAGgucccua, UAGguagacu, CAAguuaagg, GAGgugugcg, GAAgcugccc, CGAguacgug, CGGguaggua, UUGguauuga, AUUguaugau, UUGguaugaa, GAGgugguca, GCUguaugaa, CAGguguugc, CAGguaaaac, AUAguaaggu, CUGguuagag, AGCgugugag, AAGguuaucu, CACgugagua, AGGgucagua, GAGguauaau, CAGguuauuu, AGGguggacu, AUUguaauuc, UUUguggguu, AUGguacgug, AAGguguucc, CAGgugacgc, GAGguacuaa, ACAguucagu, GAGgucacgg, CAAguaaggc, AAGguuuggg, AAAgugggcu, GCGguucuug, GAGguggagc, UGAgucagug, CAGgucaagg, AGUguaagcu, GAGgcagaaa, AAGgucacac, GAAguagguu, GUCguaaguu, AGAguaugca, CCUgugcaaa, ACGgugaaaa, CAGguacgaa, CAUgugagga, AGCgugagua, GGUguguagg, AACgugagcu, GAGgugaacu, AGAguucagu, AACgugugua, CAGguugugg, AAGguacuag, UCAgugaaaa, AAUgucuggu, ACGguaaaau, CUGguguaag, GAGgugcgaa, AGGguuucuc, CAGguagccc, AUUguauugg, AUGguacuua, GAGgcccgac, UCGguaagac, CGGgcuguag, UAUgugugug, UAGguagaaa, GUGgucauua, UAGgugaaag, ACUguaauuc, GCAguacagg, UCGgugaguc, UAUguaggga, AUGguauguc, GUGgugugug, CUGgugaccu, AAUgugaaua, UAGgucucac, GAGguuauug, UGAguaggcu, CGGgcacgua, GCAguaaaua, CCGgugagag, UAAguugguc, CCGgugagcc, AAGguuguca, CUGguauuau, GGGguauggg, AAAgucagua, UUUguaugua, UAAguacugc, CAGguaccaa, GAAguucaga, AUGgugcggu, GUGgugaggu, UGAguaagcc, UAUguaaggg, GUGguggaaa, GAGgugauug, GGAguuugua, AAGgucacga, GUGguagagg, UAAguauauc, AAGgugucca, UAUgugguau, GAGguacaau, AAGguggggg, GGAguaggug, and UAGgugacuu.

In some embodiments, the splice site sequence (e.g., 5′ splice site sequence) comprises AGA. In some embodiments, the splice site sequence (e.g., 5′ splice site sequence) comprises AAA. In some embodiments, the splice site sequence (e.g., 5′ splice site sequence) comprises AAC. In some embodiments, the splice site sequence (e.g., 5′ splice site sequence) comprises AAU. In some embodiments, the splice site sequence (e.g., 5′ splice site sequence) comprises AAG. In some embodiments, the splice site sequence (e.g., 5′ splice site sequence) comprises ACA. In some embodiments, the splice site sequence (e.g., 5′ splice site sequence) comprises AUA. In some embodiments, the splice site sequence (e.g., 5′ splice site sequence) comprises AUU. In some embodiments, the splice site sequence (e.g., 5′ splice site sequence) comprises AUG. In some embodiments, the splice site sequence (e.g., 5′ splice site sequence) comprises AUC. In some embodiments, the splice site sequence (e.g., 5′ splice site sequence) comprises CAA. In some embodiments, the splice site sequence (e.g., 5′ splice site sequence) comprises CAU. In some embodiments, the splice site sequence (e.g., 5′ splice site sequence) comprises CAC. In some embodiments, the splice site sequence (e.g., 5′ splice site sequence) comprises CAG. In some embodiments, the splice site sequence (e.g., 5′ splice site sequence) comprises GAA. In some embodiments, the splice site sequence (e.g., 5′ splice site sequence) comprises GAC. In some embodiments, the splice site sequence (e.g., 5′ splice site sequence) comprises GAU. In some embodiments, the splice site sequence (e.g., 5′ splice site sequence) comprises GAG. In some embodiments, the splice site sequence (e.g., 5′ splice site sequence) comprises GGA. In some embodiments, the splice site sequence (e.g., 5′ splice site sequence) comprises GCA. In some embodiments, the splice site sequence (e.g., 5′ splice site sequence) comprises GGG. In some embodiments, the splice site sequence (e.g., 5′ splice site sequence) comprises GGC. In some embodiments, the splice site sequence (e.g., 5′ splice site sequence) comprises GUU. In some embodiments, the splice site sequence (e.g., 5′ splice site sequence) comprises GGU. In some embodiments, the splice site sequence (e.g., 5′ splice site sequence) comprises GUC. In some embodiments, the splice site sequence (e.g., 5′ splice site sequence) comprises GUA. In some embodiments, the splice site sequence (e.g., 5′ splice site sequence) comprises GUG. In some embodiments, the splice site sequence (e.g., 5′ splice site sequence) comprises UCU. In some embodiments, the splice site sequence (e.g., 5′ splice site sequence) comprises UCC. In some embodiments, the splice site sequence (e.g., 5′ splice site sequence) comprises UCA. In some embodiments, the splice site sequence (e.g., 5′ splice site sequence) comprises UCG. In some embodiments, the splice site sequence (e.g., 5′ splice site sequence) comprises UUU. In some embodiments, the splice site sequence (e.g., 5′ splice site sequence) comprises UUC. In some embodiments, the splice site sequence (e.g., 5′ splice site sequence) comprises UUA. In some embodiments, the splice site sequence (e.g., 5′ splice site sequence) comprises UUG. In some embodiments, the splice site sequence (e.g., 5′ splice site sequence) comprises UGU. In some embodiments, the splice site sequence (e.g., 5′ splice site sequence) comprises UAU. In some embodiments, the splice site sequence (e.g., 5′ splice site sequence) comprises GGA. In some embodiments, the splice site sequence (e.g., 5′ splice site sequence) comprises CUU. In some embodiments, the splice site sequence (e.g., 5′ splice site sequence) comprises CUC. In some embodiments, the splice site sequence (e.g., 5′ splice site sequence) comprises CUA. In some embodiments, the splice site sequence (e.g., 5′ splice site sequence) comprises CUG. In some embodiments, the splice site sequence (e.g., 5′ splice site sequence) comprises CCU. In some embodiments, the splice site sequence (e.g., 5′ splice site sequence) comprises CCC. In some embodiments, the splice site sequence (e.g., 5′ splice site sequence) comprises CCA. In some embodiments, the splice site sequence (e.g., 5′ splice site sequence) comprises CCG. In some embodiments, the splice site sequence (e.g., 5′ splice site sequence) comprises ACU. In some embodiments, the splice site sequence (e.g., 5′ splice site sequence) comprises ACC. In some embodiments, the splice site sequence (e.g., 5′ splice site sequence) comprises ACG. In some embodiments, the splice site sequence (e.g., 5′ splice site sequence) comprises AGC. In some embodiments, the splice site sequence (e.g., 5′ splice site sequence) comprises AGU. In some embodiments, the splice site sequence (e.g., 5′ splice site sequence) comprises AGG. In some embodiments, the splice site sequence (e.g., 5′ splice site sequence) comprises CGU. In some embodiments, the splice site sequence (e.g., 5′ splice site sequence) comprises UAC. In some embodiments, the splice site sequence (e.g., 5′ splice site sequence) comprises UAA. In some embodiments, the splice site sequence (e.g., 5′ splice site sequence) comprises UAG. In some embodiments, the splice site sequence (e.g., 5′ splice site sequence) comprises CGC. In some embodiments, the splice site sequence (e.g., 5′ splice site sequence) comprises CGA. In some embodiments, the splice site sequence (e.g., 5′ splice site sequence) comprises CGG. In some embodiments, the splice site sequence comprises AGAguaaggg.

In an embodiment, a gene sequence or splice site sequence provided herein is related to a proliferative disease, disorder, or condition (e.g., cancer, benign neoplasm, or inflammatory disease). In an embodiment, a gene sequence or splice site sequence provided herein is related to a non-proliferative disease, disorder, or condition. In an embodiment, a gene sequence or splice site sequence provided herein is related to a neurological disease or disorder; autoimmune disease or disorder; immunodeficiency disease or disorder; lysosomal storage disease or disorder; cardiovascular condition, disease or disorder; metabolic disease or disorder; respiratory condition, disease, or disorder; renal disease or disorder; or infectious disease in a subject. In an embodiment, a gene sequence or splice site sequence provided herein is related to a neurological disease or disorder (e.g., Huntington's disease). In an embodiment, a gene sequence or splice site sequence provided herein is related to an immunodeficiency disease or disorder. In an embodiment, a gene sequence or splice site sequence provided herein is related to a lysosomal storage disease or disorder. In an embodiment, a gene sequence or splice site sequence provided herein is related to a cardiovascular condition, disease or disorder. In an embodiment, a gene sequence or splice site sequence provided herein is related to a metabolic disease or disorder. In an embodiment, a gene sequence or splice site sequence provided herein is related to a respiratory condition, disease, or disorder. In an embodiment, a gene sequence or splice site sequence provided herein is related to a renal disease or disorder. In an embodiment, a gene sequence or splice site sequence provided herein is related to an infectious disease.

In an embodiment, a gene sequence or splice site sequence provided herein is related to a mental retardation disorder. In an embodiment, a gene sequence or splice site sequence provided herein is related to a mutation in the SETD5 gene. In an embodiment, a gene sequence or splice site sequence provided herein is related to an immunodeficiency disorder. In an embodiment, a gene sequence and splice site sequence provided herein is related to a mutation in the GATA2 gene.

In some embodiments, a compound of Formula (I), (III), or (V) described herein interacts with (e.g., binds to) a splicing complex component (e.g., a nucleic acid (e.g., an RNA) or a protein). In some embodiments, the splicing complex component is selected from 9G8, A1 hnRNP, A2 hnRNP, ASD-1, ASD-2b, ASF, BRR2, B1 hnRNP, C1 hnRNP, C2 hnRNP, CBP20, CBP80, CELF, F hnRNP, FBP11, Fox-1, Fox-2, G hnRNP, H hnRNP, hnRNP 1, hnRNP 3, hnRNP C, hnRNP G, hnRNP K, hnRNP M, hnRNP U, Hu, HUR, I hnRNP, K hnRNP, KH-type splicing regulatory protein (KSRP), L hnRNP, LUC7L, M hnRNP, mBBP, muscle-blind like (MBNL), NF45, NFAR, Nova-1, Nova-2, nPTB, P54/SFRS11, polypyrimidine tract binding protein (PTB), a PRP protein (e.g., PRP8, PRP6, PRP31, PRP4, PRP3, PRP28, PRP5, PRP2, PRP19), PRP19 complex proteins, RBM42, R hnRNP, RNPC1, SAD1, SAM68, SC35, SF, SF1/BBP, SF2, SF3A complex, SF3B complex, SFRS10, an Sm protein (such as B, D1, D2, D3, F, E, G), SNU17, SNU66, SNU114, an SR protein, SRm300, SRp20, SRp30c, SRP35C, SRP36, SRP38, SRp40, SRp55, SRp75, SRSF, STAR, GSG, SUP-12, TASR-1, TASR-2, TIA, TIAR, TRA2, TRA2a/b, U hnRNP, U1 snRNP, U11 snRNP, U12 snRNP, U1-70K, U1-A, U1-C, U2 snRNP, U2AF1-RS2, U2AF35, U2AF65, U4 snRNP, U5 snRNP, U6 snRNP, Urp, and YB1.

In some embodiments, the splicing complex component comprises RNA (e.g., snRNA). In some embodiments, a compound described herein binds to a splicing complex component comprising snRNA. The snRNA may be selected from, e.g., U1 snRNA, U2 snRNA, U4 snRNA, U5 snRNA, U6 snRNA, U11 snRNA, U12 snRNA, U4atac snRNA, and any combination thereof.

In some embodiments, the splicing complex component comprises a protein, e.g., a protein associated with an snRNA. In some embodiments, the protein comprises SC35, SRp55, SRp40, SRm300, SFRS10, TASR-1, TASR-2, SF2/ASF, 9G8, SRp75, SRp30c, SRp20 and P54/SFRS11. In some embodiments, the splicing complex component comprises a U2 snRNA auxiliary factor (e.g., U2AF65, U2AF35), Urp/U2AF1-RS2, SF1/BBP, CBP80, CBP 20, SF1 or PTB/hnRNP1. In some embodiments, the splicing complex component comprises a heterogenous ribonucleoprotein particle (hnRNP), e.g., an hnRNP protein. In some embodiments, the hnRNP protein comprises A1, A2/B1, L, M, K, U, F, H, G, R, I or C1/C2. Human genes encoding hnRNPs include HNRNPA0, HNRNPA1, HNRNPA1L1, HNRNPA1L2, HNRNPA3, HNRNPA2B1, HNRNPAB, HNRNPB1, HNRNPC, HNRNPCL1, HNRNPD, HNRPDL, HNRNPF, HNRNPH1, HNRNPH2, HNRNPH3, HNRNPK, HNRNPL, HNRPLL, HNRNPM, HNRNPR, HNRNPU, HNRNPUL1, HNRNPUL2, HNRNPUL3, and FMR1.

In one aspect, the compounds of Formula (I), (III), or (V) and pharmaceutically acceptable salts, solvates, hydrates, tautomers, stereoisomers, and compositions thereof, may modulate (e.g., increase or decrease) a splicing event of a target nucleic acid sequence (e.g., DNA, RNA, or a pre-mRNA), for example, a nucleic acid encoding a gene described herein, or a nucleic acid encoding a protein described herein, or a nucleic acid comprising a splice site described herein. In an embodiment, the splicing event is an alternative splicing event.

In an embodiment, the compound of Formula (I), (III), or (V) or a pharmaceutically acceptable salt, solvate, hydrate, tautomer, stereoisomer, and compositions thereof increases splicing at splice site on a target nucleic acid (e.g., an RNA, e.g., a pre-mRNA), by about 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or more, e.g., as determined by a known method in the art, e.g., qPCR. In an embodiment, the compound of Formula (I), (III), or (V) or a pharmaceutically acceptable salt, solvate, hydrate, tautomer, stereoisomer, and compositions thereof decreases splicing at splice site on a target nucleic acid (e.g., an RNA, e.g., a pre-mRNA), by about 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or more, e.g., as determined by a known method in the art, e.g., qPCR.

In another aspect, the present disclosure features a method of forming a complex comprising a component of a spliceosome (e.g., a major spliceosome component or a minor spliceosome component), a nucleic acid (e.g., a DNA, RNA, e.g., a pre-mRNA), and a compound of Formula (I), (III), or (V) or a pharmaceutically acceptable salt, solvate, hydrate, tautomer, stereoisomer, or composition thereof, comprising contacting the nucleic acid (e.g., a DNA, RNA, e.g., a pre-mRNA) with said compound of Formula (I), (III), or (V). In an embodiment, the component of a spliceosome is selected from the U1, U2, U4, U5, U6, U11, U12, U4atac, U6atac small nuclear ribonucleoproteins (snRNPs), or a related accessory factor. In an embodiment, the component of a spliceosome is recruited to the nucleic acid in the presence of the compound of Formula (I), (III), or (V), or a pharmaceutically acceptable salt, solvate, hydrate, tautomer, stereoisomer, or composition thereof.

In another aspect, the present disclosure features a method of altering the structure or conformation of a nucleic acid (e.g., a DNA, RNA, e.g., a pre-mRNA) comprising contacting the nucleic acid with a compound of Formula (I), (III), or (V) or a pharmaceutically acceptable salt, solvate, hydrate, tautomer, stereoisomer, or composition thereof In an embodiment, the altering comprises forming a bulge or kink in the nucleic acid (e.g., a DNA, RNA, e.g., a pre-mRNA). In an embodiment, the altering comprises stabilizing a bulge or a kink in the nucleic acid (e.g., a DNA, RNA, e.g., a pre-mRNA). In an embodiment, the altering comprises reducing a bulge or a kink in the nucleic acid (e.g., a DNA, RNA, e.g., a pre-mRNA). In an embodiment, the nucleic acid (e.g., a DNA, RNA, e.g., a pre-mRNA) comprises a splice site. In an embodiment, the compound of Formula (I), (III), or (V) interacts with a nucleobase, ribose, or phosphate moiety of a nucleic acid (e.g., a DNA, RNA, e.g., pre-mRNA).

The present disclosure also provides methods for the treatment or prevention of a disease, disorder, or condition. In an embodiment, the disease, disorder or condition is related to (e.g., caused by) a splicing event, such as an unwanted, aberrant, or alternative splicing event. In an embodiment, the disease, disorder or condition comprises a proliferative disease (e.g., cancer, benign neoplasm, or inflammatory disease) or non-proliferative disease. In an embodiment, the disease, disorder, or condition comprises a neurological disease, autoimmune disorder, immunodeficiency disorder, cardiovascular condition, metabolic disorder, lysosomal storage disease, respiratory condition, renal disease, or infectious disease in a subject. In another embodiment, the disease, disorder, or condition comprises a haploinsufficiency disease, an autosomal recessive disease (e.g., with residual function), or a paralogue activation disorder. In another embodiment, the disease, disorder, or condition comprises an autosomal dominant disorder (e.g., with residual function). Such methods comprise the step of administering to the subject in need thereof an effective amount of a compound of Formula (I), (III), or (V), or a pharmaceutically acceptable salt, solvate, hydrate, tautomer, stereoisomer thereof, or a pharmaceutical composition thereof. In certain embodiments, the methods described herein include administering to a subject an effective amount of a compound of Formula (I), (III), or (V), or a pharmaceutically acceptable salt thereof, or a pharmaceutical composition thereof.

In certain embodiments, the subject being treated is a mammal. In certain embodiments, the subject is a human. In certain embodiments, the subject is a domesticated animal, such as a dog, cat, cow, pig, horse, sheep, or goat. In certain embodiments, the subject is a companion animal such as a dog or cat. In certain embodiments, the subject is a livestock animal such as a cow, pig, horse, sheep, or goat. In certain embodiments, the subject is a zoo animal. In another embodiment, the subject is a research animal such as a rodent, dog, or non-human primate. In certain embodiments, the subject is a non-human transgenic animal such as a transgenic mouse or transgenic pig.

A proliferative disease, disorder, or condition may also be associated with inhibition of apoptosis of a cell in a biological sample or subject. All types of biological samples described herein or known in the art are contemplated as being within the scope of the disclosure. The compounds of Formula (I), (III), or (V) and pharmaceutically acceptable salts, solvates, hydrates, tautomers, stereoisomers, and compositions thereof, may induce apoptosis, and therefore, be useful in treating and/or preventing proliferative diseases, disorders, or conditions.

In certain embodiments, the proliferative disease to be treated or prevented using the compounds of Formula (I), (III), or (V) is cancer. As used herein, the term “cancer” refers to a malignant neoplasm (Stedman's Medical Dictionary, 25th ed.; Hensyl ed.; Williams & Wilkins: Philadelphia, 1990). All types of cancers disclosed herein or known in the art are contemplated as being within the scope of the disclosure. Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; eye cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenstrom's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendocrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic adenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

In some embodiments, the proliferative disease is associated with a benign neoplasm. For example, a benign neoplasm may include adenoma, fibroma, hemangioma, tuberous sclerosis, and lipoma. All types of benign neoplasms disclosed herein or known in the art are contemplated as being within the scope of the disclosure.

In some embodiments, the proliferative disease is associated with angiogenesis. All types of angiogenesis disclosed herein or known in the art are contemplated as being within the scope of the disclosure.

In some embodiments, the compound of Formula (I), (III), or (V), or a pharmaceutically acceptable salt thereof, or compositions comprising such compound or pharmaceutically acceptable salt thereof, is used to prevent or treat a non-proliferative disease. Exemplary non-proliferative diseases include a neurological disease, autoimmune disorder, immunodeficiency disorder, lysosomal storage disease, cardiovascular condition, metabolic disorder, respiratory condition, inflammatory disease, renal disease, or infectious disease.

In certain embodiments, the non-proliferative disease is a neurological disease. In certain embodiments, the compound of Formula (I), (III), or (V), or a pharmaceutically acceptable salt thereof, or compositions comprising such compound or pharmaceutically acceptable salt thereof, is used to prevent or treat a neurological disease, disorder, or condition. A neurological disease, disorder, or condition may include a neurodegenerative disease, a psychiatric condition, or a musculoskeletal disease. A neurological disease may further include a repeat expansion disease, e.g., which may be characterized by the expansion of a nucleic acid sequence in the genome. For example, a repeat expansion disease includes myotonic dystrophy, amyotrophic lateral sclerosis, Huntington's disease, a trinucleotide repeat disease, or a polyglutamine disorder (e.g., ataxia, fragile X syndrome). In some embodiments, the neurological disease comprises a repeat expansion disease, e.g., Huntington's disease. Additional neurological diseases, disorders, and conditions include Alzheimer's disease, Huntington's chorea, a prion disease (e.g., Creutzfeld-Jacob disease, bovine spongiform encephalopathy, Kuru, or scrapie), a mental retardation disorder (e.g., a disorder caused by a SETD5 gene mutation, e.g., intellectual disability-facial dysmorphism syndrome, autism spectrum disorder), Lewy Body disease, diffuse Lewy body disease (DLBD), dementia, progressive supranuclear palsy (PSP), progressive bulbar palsy (PBP), psuedobulbar palsy, spinal and bulbar muscular atrophy (SBMA), primary lateral sclerosis, Pick's disease, primary progressive aphasia, corticobasal dementia, Parkinson's disease, Down's syndrome, multiple system atrophy, spinal muscular atrophy (SMA), progressive spinobulbar muscular atrophy (e.g., Kennedy disease), post-polio syndrome (PPS), spinocerebellar ataxia, pantothenate kinase-associated neurodegeneration (PANK), spinal degenerative disease/motor neuron degenerative diseases, upper motor neuron disorder, lower motor neuron disorder, Hallervorden-Spatz syndrome, cerebral infarction, cerebral trauma, chronic traumatic encephalopathy, transient ischemic attack, Lytigo-bodig (amyotrophic lateral sclerosis-parkinsonism dementia), Guam-Parkinsonism dementia, hippocampal sclerosis, corticobasal degeneration, Alexander disease, Apler's disease, Krabbe's disease, neuroborreliosis, neurosyphilis, Sandhoff disease, Tay-Sachs disease, Schilder's disease, Batten disease, Cockayne syndrome, Kearns-Sayre syndrome, Gerstmann-Straussler-Scheinker syndrome and other transmissible spongiform encephalopathies, hereditary spastic paraparesis, Leigh's syndrome, a demyelinating diseases, neuronal ceroid lipofuscinoses, epilepsy, tremors, depression, mania, anxiety and anxiety disorders, sleep disorders (e.g., narcolepsy, fatal familial insomnia), acute brain injuries (e.g., stroke, head injury), autism, Machado-Joseph disease, or a combination thereof. In some embodiments, the neurological disease comprises Friedrich's ataxia or Sturge Weber syndrome. In some embodiments, the neurological disease comprises Huntington's disease. In some embodiments, the neurological disease comprises spinal muscular atrophy. All types of neurological diseases disclosed herein or known in the art are contemplated as being within the scope of the disclosure.

In certain embodiments, the non-proliferative disease is an autoimmune disorder or an immunodeficiency disorder. In certain embodiments, the compound of Formula (I), (III), or (V), or a pharmaceutically acceptable salt thereof, or compositions comprising such compound or pharmaceutically acceptable salt thereof, is used to prevent or treat an autoimmune disease, disorder, or condition, or an immunodeficiency disease, disorder, or condition. Exemplary autoimmune and immunodeficiency diseases, disorders, and conditions include arthritis (e.g., rheumatoid arthritis, osteoarthritis, gout), Chagas disease, chronic obstructive pulmonary disease (COPD), dermatomyositis, diabetes mellitus type 1, endometriosis, Goodpasture's syndrome, Graves' disease, Guillain-Barre syndrome (GBS), Hashiomoto's disease, Hidradenitis suppurativa, Kawasaki disease, ankylosing spondylitis, IgA nephropathy, idiopathic thrombocytopenic purpura, inflammatory bowel disease, Crohn's disease, ulcerative colitis, collagenous colitis, lymphocytic colitis, ischemic colitis, diversion colitis, Behcet's syndrome, infective colitis, indeterminate colitisinterstitial cystitis, lupus (e.g., systemic lupus erythematosus, discoid lupus, drug-induced lupus, neonatal lupus), mixed connective tissue disease, morphea, multiple sclerosis, myasthenia gravis, narcolepsy, neuromyotonia, pemphigus vulgaris, pernicious anemia, psoriasis, psoriatic arthritis, polymyositis, primary biliary cirrhosis, relapsing polychondritis, scleroderma, Sjögren's syndrome, Stiff person syndrome, vasculitis, vitiligo, a disorder caused by a GATA2 mutation (e.g., GATA2 deficiency; GATA2 haploinsufficiency; Emberger syndrome; monocytopenia and mycobacterium avium complex/dendritic cell, monocyte, B and NK lymphocyte deficiency; familial myelodysplastic syndrome; acute myeloid leukemia; chronic myelomonocytic leukemia), neutropenia, aplastic anemia, and Wegener's granulomatosis. In some embodiments, the autoimmune or immunodeficiency disorder comprises chronic mucocutaneous candidiasis. All types of autoimmune disorders and immunodeficiency disorders disclosed herein or known in the art are contemplated as being within the scope of the disclosure.

In certain embodiments, the non-proliferative disease is a cardiovascular condition. In certain embodiments, the compound of Formula (I), (III), or (V), or a pharmaceutically acceptable salt thereof, or compositions comprising such compound or pharmaceutically acceptable salt thereof, is used to prevent or treat a cardiovascular disease, disorder, or condition. A cardiovascular disease, disorder, or condition may include a condition relating to the heart or vascular system, such as the arteries, veins, or blood. Exemplary cardiovascular diseases, disorders, or conditions include angina, arrhythmias (atrial or ventricular or both), heart failure, arteriosclerosis, atheroma, atherosclerosis, cardiac hypertrophy, cardiac or vascular aneurysm, cardiac myocyte dysfunction, carotid obstructive disease, endothelial damage after PTCA (percutaneous transluminal coronary angioplasty), hypertension including essential hypertension, pulmonary hypertension and secondary hypertension (renovascular hypertension, chronic glomerulonephritis), myocardial infarction, myocardial ischemia, peripheral obstructive arteriopathy of a limb, an organ, or a tissue; peripheral artery occlusive disease (PAOD), reperfusion injury following ischemia of the brain, heart or other organ or tissue, restenosis, stroke, thrombosis, transient ischemic attack (TIA), vascular occlusion, vasculitis, and vasoconstriction. All types of cardiovascular diseases, disorders, or conditions disclosed herein or known in the art are contemplated as being within the scope of the disclosure.

In certain embodiments, the non-proliferative disease is a metabolic disorder. In certain embodiments, the compound of Formula (I), (III), or (V), or a pharmaceutically acceptable salt thereof, or compositions comprising such compound or pharmaceutically acceptable salt thereof, is used to prevent or treat a metabolic disease, disorder, or condition. A metabolic disease, disorder, or condition may include a disorder or condition that is characterized by abnormal metabolism, such as those disorders relating to the consumption of food and water, digestion, nutrient processing, and waste removal. A metabolic disease, disorder, or condition may include an acid-base imbalance, a mitochondrial disease, a wasting syndrome, a malabsorption disorder, an iron metabolism disorder, a calcium metabolism disorder, a DNA repair deficiency disorder, a glucose metabolism disorder, hyperlactatemia, a disorder of the gut microbiota. Exemplary metabolic conditions include obesity, diabetes (Type I or Type II), insulin resistance, glucose intolerance, lactose intolerance, eczema, hypertension, Hunter syndrome, Krabbe disease, sickle cell anemia, maple syrup urine disease, Pompe disease, and metachromatic leukodystrophy. All types of metabolic diseases, disorders, or conditions disclosed herein or known in the art are contemplated as being within the scope of the disclosure.

In certain embodiments, the non-proliferative disease is a respiratory condition. In certain embodiments, the compound of Formula (I), (III), or (V), or a pharmaceutically acceptable salt thereof, or compositions comprising such compound or pharmaceutically acceptable salt thereof, is used to prevent or treat a respiratory disease, disorder, or condition. A respiratory disease, disorder, or condition can include a disorder or condition relating to any part of the respiratory system, such as the lungs, alveoli, trachea, bronchi, nasal passages, or nose. Exemplary respiratory diseases, disorders, or conditions include asthma, allergies, bronchitis, allergic rhinitis, chronic obstructive pulmonary disease (COPD), lung cancer, oxygen toxicity, emphysema, chronic bronchitis, and acute respiratory distress syndrome. All types of respiratory diseases, disorders, or conditions disclosed herein or known in the art are contemplated as being within the scope of the disclosure.

In certain embodiments, the non-proliferative disease is a renal disease. In certain embodiments, the compound of Formula (I), (III), or (V), or a pharmaceutically acceptable salt thereof, or compositions comprising such compound or pharmaceutically acceptable salt thereof, is used to prevent or treat a renal disease, disorder, or condition. A renal disease, disorder, or condition can include a disease, disorder, or condition relating to any part of the waste production, storage, and removal system, including the kidneys, ureter, bladder, urethra, adrenal gland, and pelvis. Exemplary renal diseases include acute kidney failure, amyloidosis, Alport syndrome, adenovirus nephritis, acute lobar nephronia, tubular necrosis, glomerulonephritis, kidney stones, urinary tract infections, chronic kidney disease, polycystic kidney disease, and focal segmental glomerulosclerosis (FSGS). In some embodiments, the renal disease, disorder, or condition comprises HIV-associated nephropathy or hypertensive nephropathy. All types of renal diseases, disorders, or conditions disclosed herein or known in the art are contemplated as being within the scope of the disclosure.

In certain embodiments, the non-proliferative disease is an infectious disease. In certain embodiments, the compound of Formula (I), (III), or (V), or a pharmaceutically acceptable salt thereof, or compositions comprising such compound or pharmaceutically acceptable salt thereof, is used to prevent or treat an infectious disease, disorder, or condition. An infectious disease may be caused by a pathogen such as a virus or bacteria. Exemplary infectious diseases include human immunodeficiency syndrome (HIV), acquired immunodeficiency syndrome (AIDS), meningitis, African sleeping sickness, actinomycosis, pneumonia, botulism, chlamydia, Chagas disease, Colorado tick fever, cholera, typhus, giardiasis, food poisoning, ebola hemorrhagic fever, diphtheria, Dengue fever, gonorrhea, streptococcal infection (e.g., Group A or Group B), hepatitis A, hepatitis B, hepatitis C, herpes simplex, hookworm infection, influenza, Epstein-Barr infection, Kawasaki disease, kuru, leprosy, leishmaniasis, measles, mumps, norovirus, meningococcal disease, malaria, Lyme disease, listeriosis, rabies, rhinovirus, rubella, tetanus, shingles, scarlet fever, scabies, Zika fever, yellow fever, tuberculosis, toxoplasmosis, or tularemia. In some embodiments, the infectious disease comprises cytomegalovirus. All types of infectious diseases, disorders, or conditions disclosed herein or known in the art are contemplated as being within the scope of the disclosure.

In certain embodiments, the disease, disorder, or condition is a haploinsufficiency disease. In certain embodiments, the compound of Formula (I), (III), or (V), or a pharmaceutically acceptable salt thereof, or compositions comprising such compound or pharmaceutically acceptable salt thereof, is used to prevent or treat a haploinsufficiency disease, disorder, or condition. A haploinsufficiency disease, disorder, or condition may refer to a monogenic disease in which an allele of a gene has a loss-of-function lesion, e.g., a total loss of function lesion. In an embodiment, the loss-of-function lesion is present in an autosomal dominant inheritance pattern or is derived from a sporadic event. In an embodiment, the reduction of gene product function due to the altered allele drives the disease phenotype despite the remaining functional allele (i.e. said disease is haploinsufficient with regard to the gene in question). In an embodiment, a compound of Formula (I), (III), or (V) increases expression of the haploinsufficient gene locus. In an embodiment, a compound of Formula (I), (III), or (V) increases one or both alleles at the haploinsufficient gene locus. Exemplary haploinsufficiency diseases, disorders, and conditions include Robinow syndrome, cardiomyopathy, cerebellar ataxia, pheochromocytoma, Charcot-Marie-Tooth disease, neuropathy, Takenouchi-Kosaki syndrome, Coffin-Siris syndrome 2, chromosome 1p35 deletion syndrome, spinocerebellar ataxia 47, deafness, seizures, dystonia 9, GLUT1 deficiency syndrome 1, GLUT1 deficiency syndrome 2, stomatin-deficient cryohydrocytosis, basal cell carcinoma, basal cell nevus syndrome, medulloblastoma, somatic, brain malformations, macular degeneration, cone-rod dystrophy, Dejerine-Sottas disease, hypomyelinating neuropathy, Roussy-Levy syndrome, glaucoma, autoimmune lymphoproliferative syndrome, pituitary hormone deficiency, epileptic encephalopathy, early infantile, popliteal pterygium syndrome, van der Woude syndrome, Loeys-Dietz syndrome, Skraban-Deardorff syndrome, erythrocytosis, megalencephaly-polymicrogyria-polydactyly-hydrocephalus syndrome, mental retardation, CINCA syndrome, familial cold inflammatory syndrome 1, keratoendothelitis fugax hereditaria, Muckle-Wells syndrome, Feingold syndrome 1, Acute myeloid leukemia, Heyn-Sproul-Jackson syndrome, Tatton-Brown-Rahman syndrome, Shashi-Pena syndrome, Spastic paraplegia, autosomal dominant, macrophthalmia, colobomatous, with microcornea, holoprosencephaly, schizencephaly, endometrial cancer, familial, colorectal cancer, hereditary nonpolyposis, intellectual developmental disorder with dysmorphic facies and behavioral abnormalities, ovarian hyperstimulation syndrome, schizophrenia, Dias-Logan syndrome, premature ovarian failure, dystonia, dopa-responsive, due to sepiapterin reductase deficiency, Beck-Fahrner syndrome, chromosome 2p12-p11.2 deletion syndrome, neuronopathy, spastic paraplegia, familial adult myoclonic, colorectal cancer, hypothyroidism, Culler-Jones syndrome, holoprosencephaly, myelokathexis, WHIM syndrome, Mowat-Wilson syndrome, mental retardation, an intellectual developmental disorder, autism spectrum disorder, epilepsy, epileptic encephalopathy, Dravet syndrome, migraines, a mental retardation disorder (e.g., a disorder caused by a SETD5 gene mutation, e.g., intellectual disability-facial dysmorphism syndrome, autism spectrum disorder), a disorder caused by a GATA2 mutation (e.g., GATA2 deficiency; GATA2 haploinsufficiency; Emberger syndrome; monocytopenia and mycobacterium avium complex/dendritic cell, monocyte, B and NK lymphocyte deficiency; familial myelodysplastic syndrome; acute myeloid leukemia; chronic myelomonocytic leukemia), and febrile seizures.

In certain embodiments, the disease, disorder, or condition is an autosomal recessive disease, e.g., with residual function. In certain embodiments, the compound of Formula (I), (III), or (V), or a pharmaceutically acceptable salt thereof, or compositions comprising such compound or pharmaceutically acceptable salt thereof, is used to prevent or treat an autosomal recessive disease, disorder, or condition. An autosomal recessive disease with residual function may refer to a monogenic disease with either homozygous recessive or compound heterozygous heritability. These diseases may also be characterized by insufficient gene product activity (e.g., a level of gene product greater than 0%). In an embodiment, a compound of Formula (I), (III), or (V) may increase the expression of a target (e.g., a gene) related to an autosomal recessive disease with residual function. Exemplary autosomal recessive diseases with residual function include Friedreich's ataxia, Stargardt disease, Usher syndrome, chlorioderma, fragile X syndrome, achromatopsia 3, Hurler syndrome, hemophilia B, alpha-1-antitrypsin deficiency, Gaucher disease, X-linked retinoschisis, Wiskott-Aldrich syndrome, mucopolysaccharidosis (Sanfilippo B), DDC deficiency, epidermolysis bullosa dystrophica, Fabry disease, metachromatic leukodystrophy, and odontochondrodysplasia.

In certain embodiments, the disease, disorder, or condition is an autosomal dominant disease. In certain embodiments, the compound of Formula (I), (III), or (V), or a pharmaceutically acceptable salt thereof, or compositions comprising such compound or pharmaceutically acceptable salt thereof, is used to prevent or treat an autosomal dominant disease, disorder, or condition. An autosomal dominant disease may refer to a monogenic disease in which the mutated gene is a dominant gene. These diseases may also be characterized by insufficient gene product activity (e.g., a level of gene product greater than 0%). In an embodiment, a compound of Formula (I), (III), or (V) may increase the expression of a target (e.g., a gene) related to an autosomal dominant disease. Exemplary autosomal dominant diseases include Huntington's disease, achondroplasia, antithrombin III deficiency, Gilbert's disease, Ehlers-Danlos syndrome, hereditary hemorrhagic telangiectasia, intestinal polyposis, hereditary elliptosis, hereditary spherocytosis, marble bone disease, Marfan's syndrome, protein C deficiency, Treacher Collins syndrome, Von Willebrand's disease, tuberous sclerosis, osteogenesis imperfecta, polycystic kidney disease, neurofibromatosis, and idiopathic hypoparathyroidism.

In certain embodiments, the disease, disorder, or condition is a paralogue activation disorder. In certain embodiments, the compound of Formula (I), (III), or (V), or a pharmaceutically acceptable salt thereof, or compositions comprising such compound or pharmaceutically acceptable salt thereof, is used to prevent or treat a paralogue activation disease, disorder, or condition. A paralogue activation disorder may comprise a homozygous mutation of genetic locus leading to loss-of-function for the gene product. In these disorders, there may exist a separate genetic locus encoding a protein with overlapping function (e.g. developmental paralogue), which is otherwise not expressed sufficiently to compensate for the mutated gene. In an embodiment, a compound of Formula (I), (III), or (V) activates a gene connected with a paralogue activation disorder (e.g., a paralogue gene).

The cell described herein may be an abnormal cell. The cell may be in vitro or in vivo. In certain embodiments, the cell is a proliferative cell. In certain embodiments, the cell is a cancer cell. In certain embodiments, the cell is a non-proliferative cell. In certain embodiments, the cell is a blood cell. In certain embodiments, the cell is a lymphocyte. In certain embodiments, the cell is a benign neoplastic cell. In certain embodiments, the cell is an endothelial cell. In certain embodiments, the cell is an immune cell. In certain embodiments, the cell is a neuronal cell. In certain embodiments, the cell is a glial cell. In certain embodiments, the cell is a brain cell. In certain embodiments, the cell is a fibroblast. In certain embodiment, the cell is a primary cell, e.g., a cell isolated from a subject (e.g., a human subject).

In certain embodiments, the methods described herein comprise the additional step of administering one or more additional pharmaceutical agents in combination with the compound of Formula (I), (III), or (V), a pharmaceutically acceptable salt thereof, or compositions comprising such compound or pharmaceutically acceptable salt thereof. Such additional pharmaceutical agents include, but are not limited to, anti-proliferative agents, anti-cancer agents, anti-diabetic agents, anti-inflammatory agents, immunosuppressant agents, and a pain-relieving agent The additional pharmaceutical agent(s) may synergistically augment the modulation of splicing induced by the inventive compounds or compositions of this disclosure in the biological sample or subject. Thus, the combination of the inventive compounds or compositions and the additional pharmaceutical agent(s) may be useful in treating, for example, a cancer or other disease, disorder, or condition resistant to a treatment using the additional pharmaceutical agent(s) without the inventive compounds or compositions.

### EXAMPLES

In order that the invention described herein may be more fully understood, the following examples are set forth. The examples described in this application are offered to illustrate the compounds, pharmaceutical compositions, and methods provided herein and are not to be construed in any way as limiting their scope.

The compounds provided herein can be prepared from readily available starting materials using modifications to the specific synthesis protocols set forth below that would be well known to those of skill in the art. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvents used, but such conditions can be determined by those skilled in the art by routine optimization procedures.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in Greene et al., Protecting Groups in Organic Synthesis, Second Edition, Wiley, New York, 1991, and references cited therein.

Reactions can be purified or analyzed according to any suitable method known in the art. For example, product formation can be monitored by spectroscopic means, such as nuclear magnetic resonance (NMR) spectroscopy (e.g., ¹H or ¹³C), infrared (IR) spectroscopy, spectrophotometry (e.g., UV-visible), mass spectrometry (MS), or by chromatographic methods such as high performance liquid chromatography (HPLC) or thin layer chromatography (TLC).

Proton NMR: ¹H NMR spectra were recorded in CDCl₃ solution in 5-mm o.d. tubes (Wildmad) at 24° C. and were collected on a BRUKER AVANCE NEO 400 at 400 MHz for ¹H. The chemical shifts (δ) are reported relative to tetramethylsilane (TMS=0.00 ppm) and expressed in ppm.

LC/MS: Liquid chromatography-mass spectrometry (LC/MS) was performed on Shimadzu-2020EV using column: Shim-pack XR-ODS (C18, Ø4.6×50 mm, 3 μm, 120 Å, 40° C.) operating in ESI(+) ionization mode; flow rate=1.2 mL/min. Mobile phase=0.05% TFA in water or CH₃CN; or on Shimadzu-2020EV using column : Poroshell HPH-C18 (C18, Ø4.6×50 mm, 3 μm, 120 Å, 40° C.) operating in ESI(+) ionization mode; flow rate=1.2 mL/min. Mobile phase A: Water/5mM NH₄HCO₃, Mobile phase B: CH₃CN.)

Analytical chiral HPLC: Analytical chiral HPLC was performed on a Agilent 1260 using column: CHIRALPAK IG-3, CHIRALPAK IC-3 or CHIRALPAK OJ-3, with flow rate=1.2 mL/min. Mobile phase=MTBE(DEA):EtOH=50:50).

Preparative HPLC purification: prep-HPLC purification was performed on a Waters-2545 or Shimadzu, using column: X-Select CSH C18 OBD (130 Å, 5 μm, 30 mm×150 mm), Xridge Prep OBD C18 (30×150 mm, 5 μm), XBridge Prep C18 OBD (Sum, 19 mm×150 mm), or YMC-Actus Triart C18 (30×150 mm, 5 μm).

Condition 1: Column: YMC-Actus Triart C18, 30×150 mm, 5 μm; Mobile Phase A: water (10 mmol/L NH₄HCO₃), Mobile Phase B: ACN; Flow rate:60 mL/min; Gradient 1:10% B up to 40% B in 8 min.

Preparative chiral HPLC: purification by chiral HPLC was performed on a Gilson-GX 281 using column: CHIALPAK IG-3, CHIRALPAK IC-3 or CHIRALPAK OJ-3.

### General Synthetic Schemes

Compounds of the present disclosure may be prepared using a synthetic protocol illustrated below in Schemes A and B.

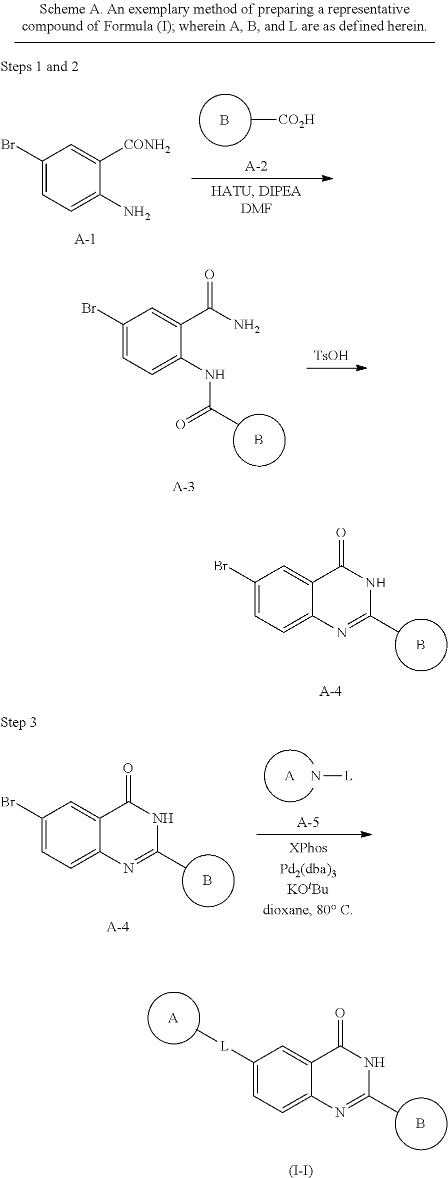

An exemplary method of preparing a compound of Formula (I-I) is provided in Scheme A. In this scheme, A-3 is prepared in Step 1 by incubating A-1 with A-2 in the presence of hexafluorophosphate azabenzotriazole tetramethyl uranium (HATU), or a similar coupling agent, diisopropylethylamine (DIPEA), and dimethylformamide (DMF). Suitable alternatives to DIPEA and DMF may also be used in the reaction. In Step 2, A-3 is cyclized by treatment with tosic acid, or a similar alternative, in order to provide A4.

Next, in Step 3, A-4 is coupled with A-5 to provide a compound of Formula (I-I). This coupling reaction may be conducted in the presence of Pd₂(dba)₃, XPhos, and KOtBu or a similar reagent. Alternative catalysts to Pd₂(dba)₃ may also be used, such as any suitable palladium catalyst. Likewise, other ligands similar to XPhos may be implemented in the reaction of Step 3. The reaction of Step 3 is carried out in dioxane, or a similar solvent, and the reaction is heated to 80° C. or a temperature sufficient to provide the compound of Formula (I-I). Each starting material and/or intermediate in Scheme A may be protected and deprotected using standard protecting group methods. In addition, purification and characterization of each intermediate as well as the final compound of Formula (I) may be afforded by any accepted procedure.

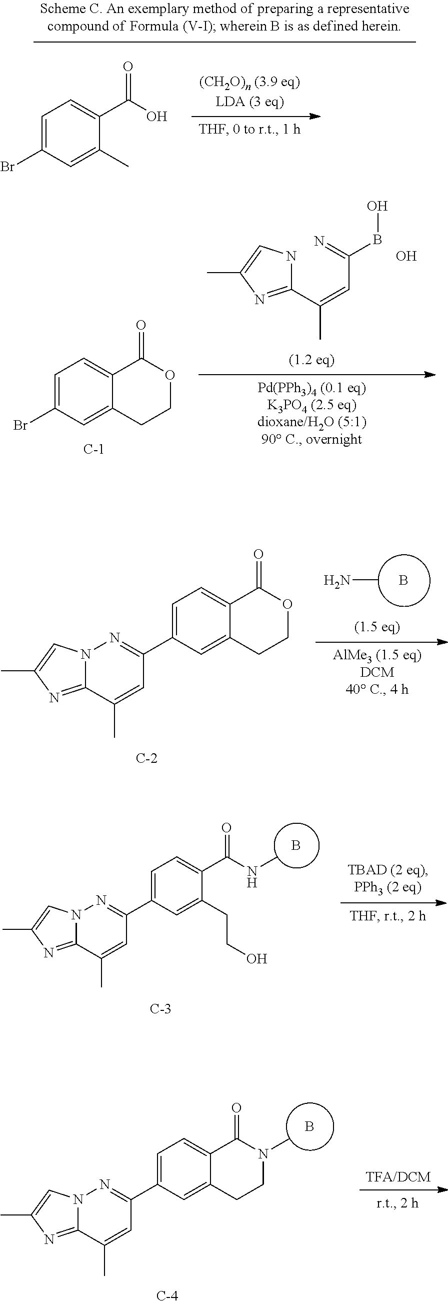

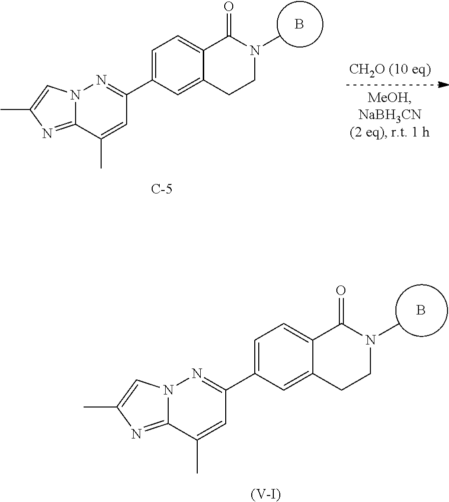

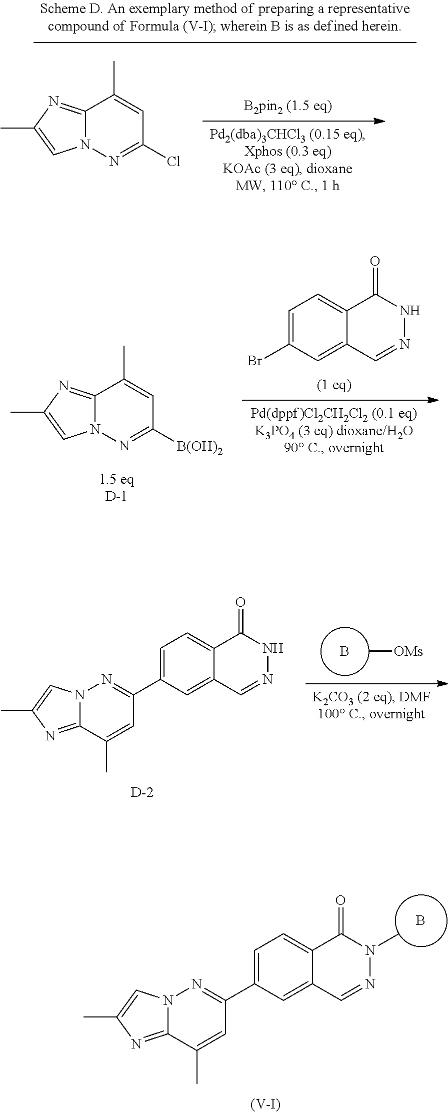

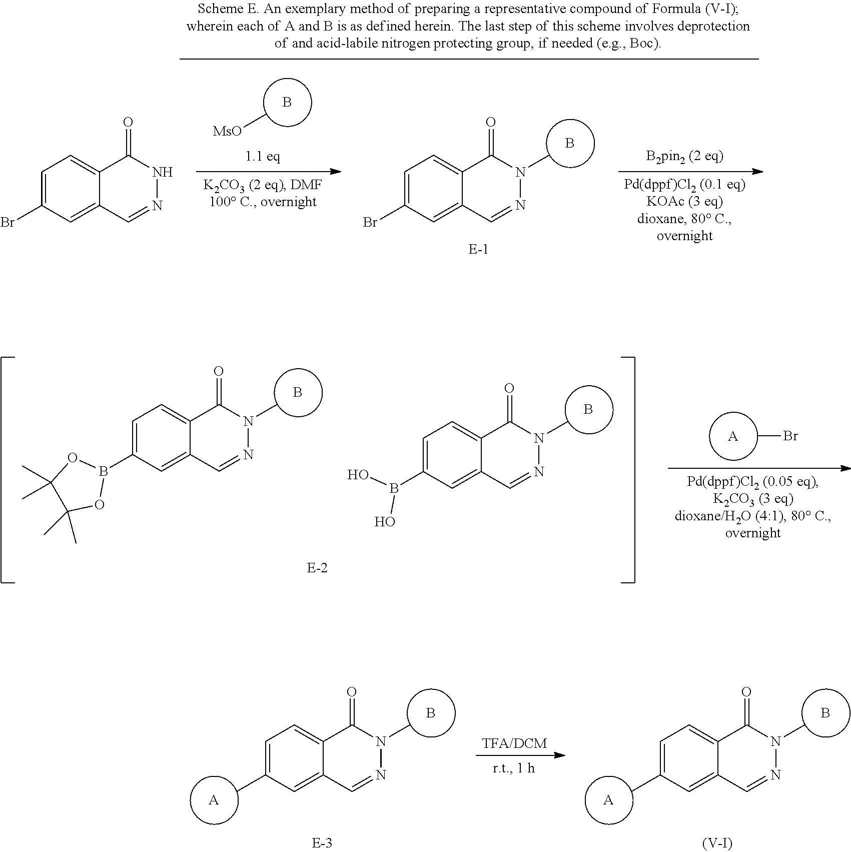

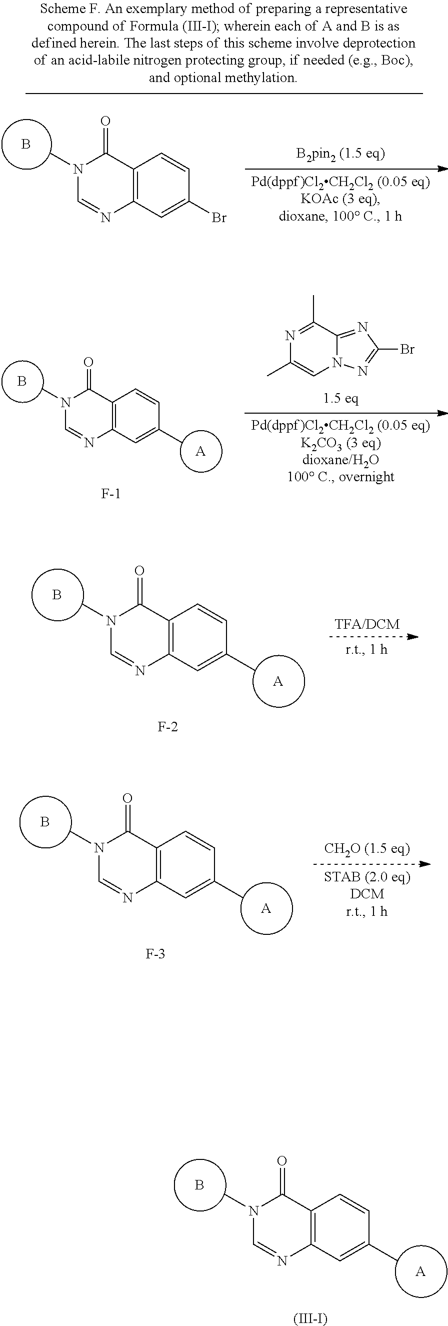

### Example 1

### Synthesis of Compound 108

### Synthesis of Intermediate B2

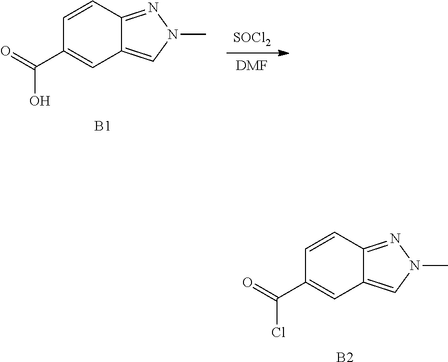

A mixture of 2-methyl-2H-indazole-5-carboxylic acid (B1; 230 mg, 1.3 mmol) in thionyl chloride (4 mL) was stirred at room temperature for 16 h. Additional thionyl chloride (2 mL) was then added dropwise, followed by one drop of DMF, and the reaction mixture was stirred at 80° C. for 4 h. The resulting solution was cooled and concentrated, and toluene (3 mL) was added. The resulting suspension was concentrated to dryness, and then co-evaporated with toluene two more times, to afford 2-methyl-2H-indazole-5-carboxylic acid (B2; 254 mg, 1.3 mmol), which was used in the next step without further purification.

### Synthesis of Intermediate B4

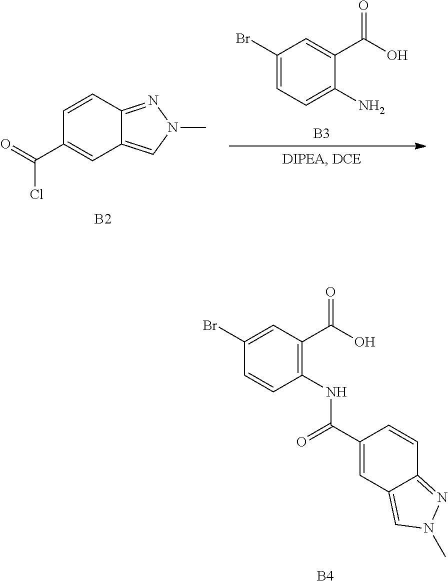

2-Amino-5-bromobenzoic acid (B3; 279 mg, 1.3 mmol) was added to methyl-2H-indazole-6-carbonyl chloride (B2; 254 mg, 1.3 mmol) in dichloroethane (13 mL) at 0° C., followed by diisopropylethylamine (250 μL, 1.43 mmol). The reaction was then warmed to room temperature and stirred for 16 h, and then concentrated, to afford 5-bromo-2-(2-methyl-2H-indazole-5-carboxamido)benzoic acid (B4) which used in the next step without further purification. LCMS (ES, m/z): 374.0 [M+H]⁺.

### Synthesis of Intermediate B5

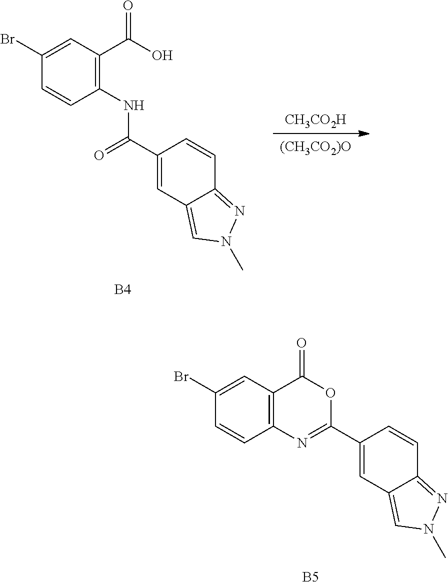 [SMILES: Cn1cc2cc(C(=O)Nc3ccc(Br)cc3C(=O)O)ccc2n1 ; "B4" ; "(CH3CO2)O" ; "CH3CO2H" ; Cn1cc2cc(-c3nc4ccc(Br)cc4c(=O)o3)ccc2n1 ; "B5"]

5-Bromo-2-(2-methyl-2H-indazole-5-carboxamido)benzoic acid (B4) was added to a mixture of acetic acid (1.5 mL) and acetic anhydride (1.5 mL) and stirred at 100° C. for 3 h, then poured into ice. The resulting precipitate was collected by filtration, rinsed with water, and dried to afford 6-bromo-2-(2-methyl-2H-indazol-5-yl)-4H-benzo[d][1,3]oxazin-4-one (B5; 380 mg) as a solid. LCMS (ES, m/z): 355.8 [M+H]⁺. ¹H NMR (DMSO-d₆, 400 MHz): δ_{H} 8.67 (1H, s), 8.62 (1H, s), 8.22 (1H, s), 8.08 (1H, d, J=8.7 Hz), 8.04 (1H, d, J=9.3 Hz), 7.73 (1H, d, J=9.2 Hz), 7.64 (1H, d, J=8.6 Hz), 4.21 (3H, s).

### Synthesis of Intermediate B6

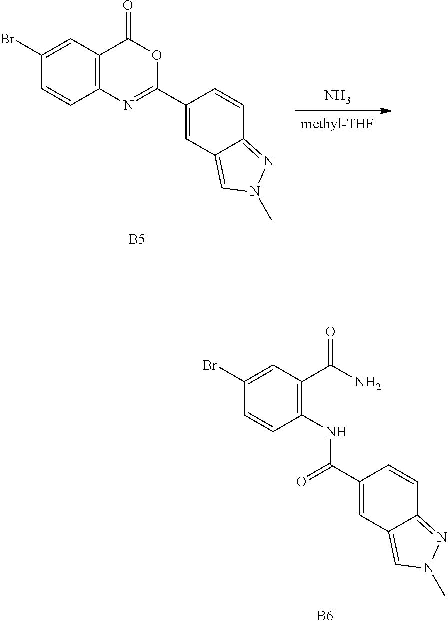

A suspension of 6-bromo-2-(2-methyl-2H-indazol-5-yl)-4H-benzo[d][1,3]oxazin-4-one (B5; 200 mg, 0.56 mmol) in 2-methyltetrahydrofuran (10 mL) was cooled in an ice bath, and ammonia was bubbled through the mixture for 5 minutes. The reaction mixture was then concentrated to dryness, to afford N-(4-bromo-2-carbamoylphenyl)-2-methyl-2H-indazole-5-carboxamide (B6) which was used in the next step without further purification. LCMS (ES, m/z): 373.0 [M+H]⁺.

### Synthesis of Intermediate B7

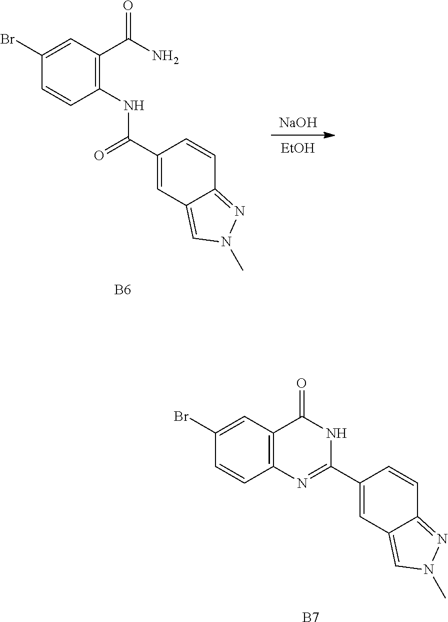 [SMILES: "EtOH" ; Cn1cc2cc(C(=O)Nc3ccc(Br)cc3C(N)=O)ccc2n1 ; "B6" ; "NaOH" ; Cn1cc2cc(-c3nc4ccc(Br)cc4c(=O)[nH]3)ccc2n1 ; "B7"]

A 2N solution of sodium hydroxide (2.3 mL, 4.6 mmol) was added to a suspension of N-(4-bromo-2-carbamoylphenyl)-2-methyl-2H-indazole-5-carboxamide (B6; 190 mg, 0.51 mmol) in ethanol (11.2 mL) and stirred for 16 h. The mixture was then concentrated under vacuum and acidified with 6N HCl to achieve a pH of 6. The resulting solid was collected by filtration and dried, to afford 6-bromo-2-(2-methyl-2H-indazol-5-yl)quinazolin-4(3H)-one (B7; 165 mg) as a solid. LCMS (ES, m/z): 354.9 [M+H]⁺. ¹H NMR (DMSO-d₆, 400 MHz): δ_{H} 12.66 (1H, s), 8.64 (1H, s), 8.59 (1H, s), 8.21 (1H, s), 8.06 (1H, d, J=9.2 Hz), 7.96 (1H, d, J=8.5 Hz), 7.69 (2H, t, J=8.6 Hz), 4.20 (3H, s).

### Synthesis of Compound 108

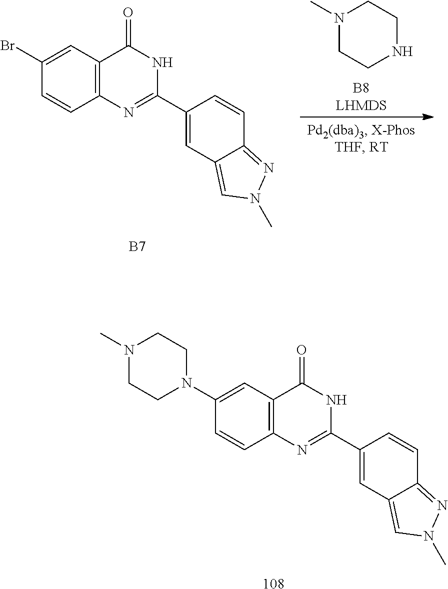

A mixture of 6-bromo-2-(2-methyl-2H-indazol-5-yl)quinazolin-4(3H)-one (B7; 75 mg, 0.21 mmol), Pd₂(dba)₃ (19.3 mg, 0.021 mmol), and X-Phos (20.1 mg, 0.042 mmol) was evacuated and purged with argon three times. Anhydrous tetrahydrofuran (10.5 mL) was then added, and argon was bubbled through the suspension for 5 minutes. N-Methyl piperazine (B8) was then added and the reaction was stirred for 10 min, after which LiHMDS (1M in tetrahydrofuran; 1 mL) was added dropwise and the reaction was stirred for 16 h, and then cooled to 0° C. and quenched with water. The pH was adjusted to 7 with a 1N solution of HCl, and the mixture was concentrated. The aqueous phase was extracted three times with dichloromethane, and then concentrated to dryness. The resulting solid was stirred in dichloromethane/methanol (9/1; 20 mL), filtered, and further rinsed with dichloromethane/methanol (9/1). The filtrate was concentrated and purified by silica gel column chromatography eluting with methanol in dichloromethane (15 to 30%). The recovered material was stirred in ethyl acetate (5 mL), filtered, and rinsed with cold ethyl acetate, to afford 2-(2-methyl-2H-indazol-5-yl)-6-(4-methylpiperazin-1-yl)quinazolin-4(3H)-one (Compound 108; 22 mg) as a solid. LCMS (ES, m/z): 375.2 [M+H]⁺. ¹H NMR (CH₃OH-d4, 400 MHz): δ_{H} 8.41 (2H, s), 8.00 (1H, d, J=9.2 Hz), 7.71-7-75 (2H, m), 7.61-7.63 (2H, m), 4.26 (3H, s), 3.43 (4H, s), 2.88 (4H, s), 2.54 (3H, s). Note: Signal of the amide-NH hydrogen atom exchanges with the residual water from the CH₃OH-d₄.

### Example 2

### Synthesis of Compound 152

### Synthesis of Intermediate B16

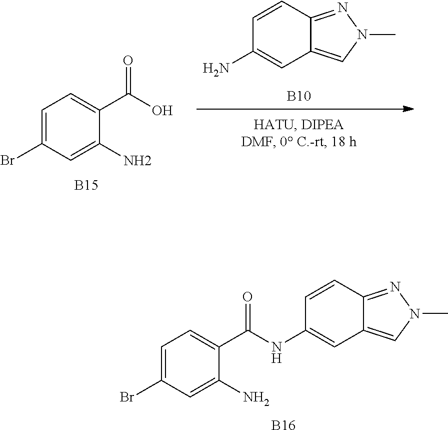

Diisopropylethylamine (1.6 mL, 9 mmol) and HATU (1.37 g, 3.6 mmol) were added sequentially to a solution of 2-amino-4-bromobenzoic acid (B15; 650 mg, 3 mmol) and 2-methyl-2H-indazol-5-amine (B10; 465 mg, 3.2 mmol) in dimethylformamide (14 mL), and the mixture was stirred at 0° C. for 1 h, then warmed to room temperature and stirred overnight. Ethyl acetate (100 mL) and a saturated solution of ammonium chloride (100 mL) were added to the mixture, and the organic layer was separated and washed with a saturated solution of ammonium chloride (50 mL), saturated sodium bicarbonate (50 mL), and brine (50 mL), then dried over magnesium sulfate, filtered, and concentrated under reduced pressure, to afford 2-amino-4-bromo-N-(2-methyl-2H-indazol-5-yl)benzamide (B16; 1.1 g) as a solid. LCMS (ES, m/z): 345.0 [M+H]⁺.

### Synthesis of Intermediate B17

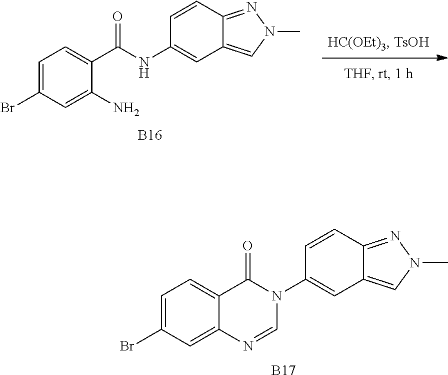

Triethyl orthoformate (4.38 g, 29.6 mmol) and tosic acid (60 mg, 0.3 mmol) were added to a solution of 2-amino-4-bromo-N-(2-methyl-2H-indazol-5-yl)benzamide (B16; 1 g, 3 mmol) in tetrahydrofuran (5 mL), and the resulting mixture was stirred at room temperature for 1 h. Ethyl acetate (50 mL) was then added, and the mixture was washed with saturated sodium bicarbonate (2×50 mL) and brine (50 mL), then dried over magnesium sulfate, filtered and concentrated under reduced pressure, to afford 7-bromo-3-(2-methyl-2H-indazol-5-yl)quinazolin-4(3H)-one (B17; 0.87 g) as a solid. LCMS (ES, m/z): 355.0 [M+H]⁺.

### Synthesis of Compound 152

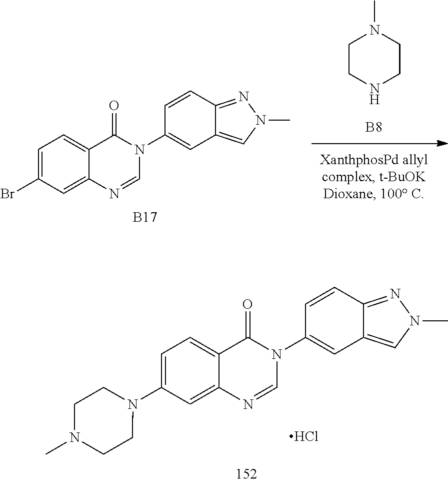

A mixture of 7-bromo-3-(2-methyl-2H-indazol-5-yl)quinazolin-4(3H)-one (B17; 100 mg, 0.28 mmol), 1-methylpiperazine (B8; 84 mg, 0.85 mmol), Xantphos-Pd-Allyl complex (21 mg, 0.03 mmol) and potassium tert-butoxide (47 mg, 0.42 mmol) in dioxane (5 mL) was heated to 100° C. for 24 h, and then cooled to room temperature and diluted with dichloromethane. The resulting mixture was filtered through Celite, and the filtrate was concentrated under reduced pressure and purified by reverse phase chromatography eluting with acetonitrile in a 0.1% aqueous HCl solution (using a gradient of 5 to 50% acetonitrile), to afford 3-(2-methyl-2H-indazol-5-yl)-7-(4-methylpiperazin-1-yl)quinazolin-4(3H)-one (Compound 152; 25 mg) as an HCl salt. LCMS (ES, m/z): 375.2 [M+H]⁺. ¹H NMR (CH₃OH-d₄, 400 MHz): δ_{H} 9.35 (1H, s), 8.41 (1H, s), 8.23 (1H, d, J=9.1 Hz), 7.95 (1H, s), 7.79 (1H, d, J=9.1 Hz), 7.74 (1H, s), 7.42 (2H, t, J=7.4 Hz), 7.15 (1H, s), 5.05 (5H, m), 3.68 (2H, d, J=12.3 Hz), 3.46 (2H, t, J=13.2 Hz), 3.29 (2H, m), 2.98 (3H, s).

### Example 3

### Synthesis of Compound 153

### Synthesis of Intermediate B19

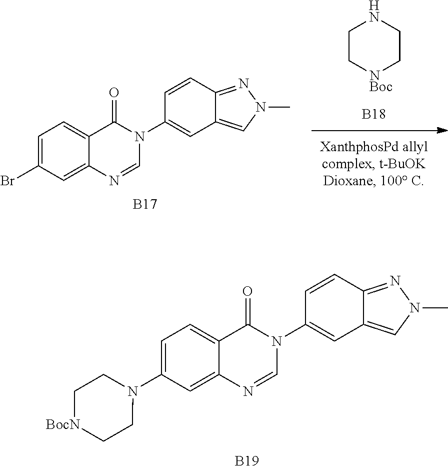

A mixture of 7-bromo-3-(2-methyl-2H-indazol-5-yl)quinazolin-4(3H)-one (B17 from Example 3; 100 mg, 0.28 mmol), tert-butyl piperazine-1-carboxylate (B18; 157 mg, 0.85 mmol), Xantphos-Pd-Allyl complex (21 mg, 0.03 mmol) and potassium tert-butoxide (47 mg, 0.42 mmol) in dioxane (5 mL) was heated to 100° C. for 24 h and then cooled to room temperature and diluted with dichloromethane. The mixture was filtered through Celite and concentrated under reduced pressure, to afford crude tert-butyl 4-(3-(2-methyl-2H-indazol-5-yl)-4-oxo-3,4-dihydroquinazolin-7-yl)piperazine-1-carboxylate (B19; 110 mg) which was used in the next step without further purification. LCMS (ES, m/z): 461.3 [M+H]⁺.

### Synthesis of Compound 153

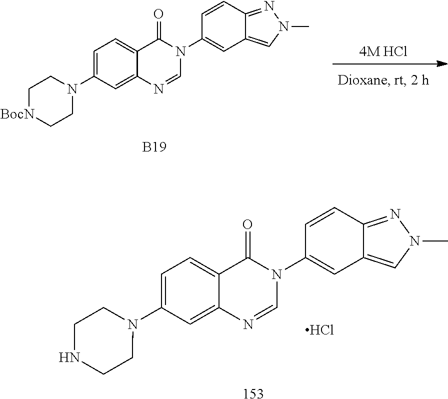

A mixture of tert-butyl 4-(3-(2-methyl-2H-indazol-5-yl)-4-oxo-3,4-dihydroquinazolin-7-yl)piperazine-1-carboxylate (B19; 110 mg, 0.31 mmol) and 4M HCl in dioxane (3 mL, 12 mmol) was stirred at room temperature for 2 h. The volatiles were then removed under reduced pressure and the crude product was purified by reverse phase chromatography eluting with acetonitrile in a 0.1% aqueous HCl solution (using a gradient of 5 to 50% acetonitrile), to afford 3-(2-methyl-2H-indazol-5-yl)-7-(piperazin-1-yl)quinazolin-4(3H)-one (Compound 153; 34 mg) as an HCl salt. LCMS (ES, m/z): 361.3 [M+H]⁺. ¹H NMR (DMSO-d₆, 400 MHz): δ_{H} 8.94-8.99 (2H, m), 8.47 (1H, s), 8.32 (1H, s), 8.03 (1H, d, J=8.9 Hz), 7.83 (1H, s), 7.69 (1H, d, J=9.1 Hz), 7.28 (2H, t, J=8.9 Hz), 7.11 (1H, s), 4.20 (3H, s), 3.63 (4H, s), 3.24 (4H, s).

### Example 4

### Synthesis of Compound 156

### Synthesis of Intermediate B21

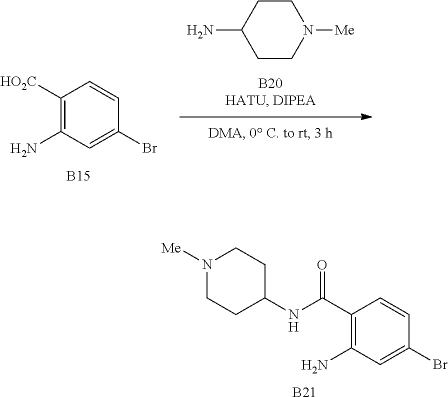

A mixture of 2-amino-4-bromobenzoic acid (B15; 200 mg, 0.93 mmol) and 4-amino-1-methylpiperidine (B20; 120 mg, 1.05 mmol) in dimethylacetamide (4.6 mL) was cooled to 0° C. Diisopropylethylamine (500 μL, 2.86 mmol) was then added dropwise, followed by HATU (388 mg, 1 mmol), and the resulting mixture was stirred at room temperature for 3 h. Ethyl acetate (25 mL) was then added, and the mixture was washed with saturated aqueous ammonium chloride (25 mL), followed by saturated aqueous sodium bicarbonate (25 mL), and brine (25 mL). The organic layer was dried over anhydrous sodium sulfate and concentrated in vacuo to afford 2-amino-4-bromo-N-(1-methylpiperidin-4-yl)benzamide (B21; 199 mg, 0.64 mmol) as a solid. LCMS (ES, m/z): 312.1 [M+H]⁺.

### Synthesis of Intermediate B22

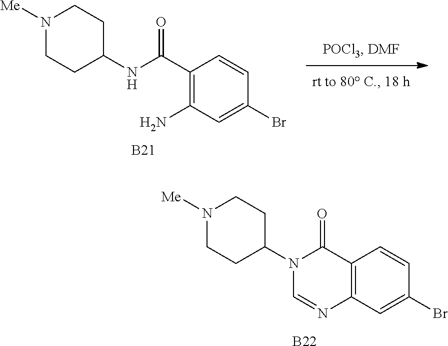

Phosphoryl chloride (0.85 mL, 6.29 mmol) was added to dimethylformamide (0.48 mL) under a nitrogen atmosphere, and the resulting mixture was stirred at room temperature for 40 min. 2-Amino-4-bromo-N-(1-methylpiperidin-4-yl)benzamide (B21; 65 mg, 0.21 mmol) was then added, and the reaction mixture was stirred at room temperature for 30 min, and then heated to 80° C. overnight. The mixture was then diluted with ethyl acetate (15 mL) and saturated aqueous sodium bicarbonate (15 mL). The aqueous layer was washed with ethyl acetate (3×15 mL), and the combined organic layers were dried over anhydrous sodium sulfate and concentrated in vacuo, to afford 7-bromo-3-(1-methylpiperidin-4-yl)quinazolin-4(3H)-one (B22; 55 mg, 0.17 mmol) as an oil. LCMS (ES, m/z): 322.1 [M+H]⁺.

### Synthesis of Compound 156

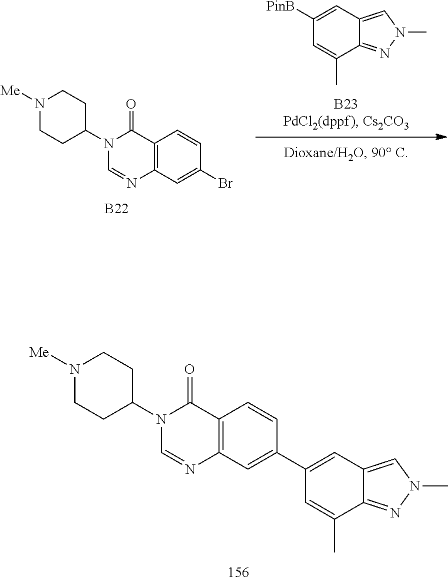

A mixture of 7-bromo-3-(1-methylpiperidin-4-yl)quinazolin-4(3H)-one (B22; 55 mg, 0.15 mmol), 2,7-dimethyl-5-(4,4,5,5-tetramethyl-1,3,2-dioxaborolan-2-yl)-2H-indazole (B23; 51 mg, 0.16 mmol), PdCl₂(dppf) (14 mg, 0.015 mmol) and Cs₂CO₃ (111 mg, 0.29 mmol) in dioxane (2.5 mL) and H₂O (0.2 mL) was heated to 90° C. for 16 h and then cooled to room temperature. The reaction mixture was dissolved in dimethylformamide and filtered through Celite using dimethylformamide as an eluent. The filtrate was concentrated under vacuum, diluted with 1M aqueous HCl (20 mL), and washed with dichloromethane (3×15 mL). The aqueous layer was filtered under vacuum and neutralized with sodium carbonate, and the resulting suspension was extracted with dichloromethane (3×15 mL). The organic layer was dried over sodium sulfate and concentrated in vacuo to afford 7-(2,7-dimethyl-2H-indazol-5-yl)-3-(1-methylpiperidin yl)quinazolin-4(3H)-one (Compound 156; 53 mg, 0.14 mmol) as a solid. LCMS (ES, m/z): 388.2 [M+H]⁺. ¹H NMR (CH₃OH-d₄, 400 MHz): δ 8.40 (1H, s), 8.28 (2H, s), 7.91 (3H, s), 7.47 (1H, s), 4.75 (1H, m) 4.25 (3H, s), 3.07 (2H, d, J=11.7 Hz), 2.64 (3H, s), 2.37 (3H, s), 2.27 (2H, t, J=12.3 Hz), 2.16 (2H, d, J=15.0 Hz), 1.97 (2H, d, J=11.8 Hz).

### Example 5

### Synthesis of Compound 157

### Synthesis of Intermediate B24

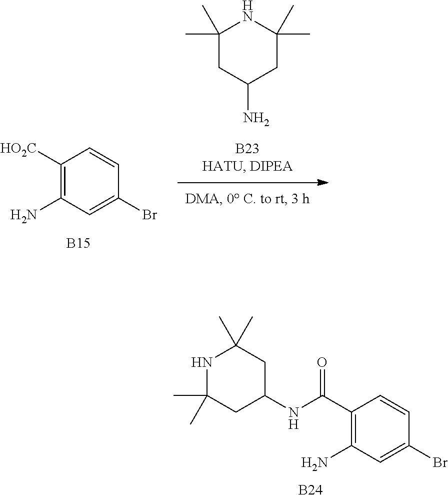

A mixture of 2-amino-4-bromobenzoic acid (B15; 100 mg, 0.46 mmol) and 2,2,6,6-tetramethylpiperidin-4-amine (B23; 80 mg, 0.51 mmol) in dimethylacetamide (2.3 mL) was cooled to 0° C., then diisopropylethylamine (250 μL, 1.43 mmol) was added dropwise followed by HATU (194 mg, 0.51 mmol). The mixture was then stirred at room temperature 3 h, and then diluted with ethyl acetate (20 mL) and washed with saturated aqueous ammonium chloride (20 mL), followed by saturated aqueous sodium bicarbonate (20 mL), and brine (20 mL). The organic layer was dried over anhydrous sodium sulfate, and concentrated in vacuo, to afford 2-amino-4-bromo-N-(2,2,6,6-tetramethylpiperidin-4-yl)benzamide (B24; 153 mg, 0.43 mmol) as a solid. LCMS (ES, m/z): 354.1 [M+H]⁺.

### Synthesis of Intermediate B25

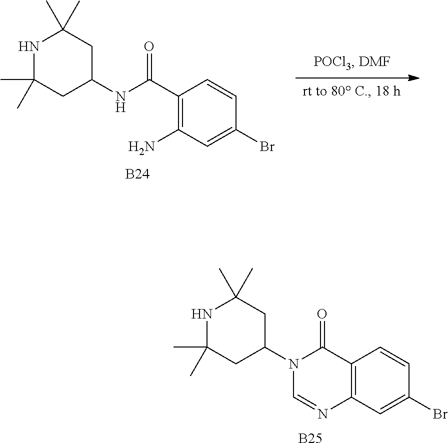

Phosphoryl chloride (0.86 mL, 6.37 mmol) was added to dimethylformamide (0.53 mL) under a nitrogen atmosphere, and the resulting solution was stirred at room temperature for 40 minutes. 2-Amino-4-bromo-N-(2,2,6,6-tetramethylpiperidin-4-yl)benzamide (B24; 69 mg, 0.2 mmol) was then added, and the resulting mixture was stirred at room temperature for 40 minutes, and then heated to 80° C. overnight. The reaction mixture was diluted with ethyl acetate (15 mL) and saturated aqueous sodium bicarbonate (15 mL). The aqueous layer was washed with ethyl acetate (3×15 mL), and the combined organic layers were dried over anhydrous sodium sulfate and concentrated in vacuo, to afford 7-bromo-3-(2,2,6,6-tetramethylpiperidin-4-yl)quinazolin-4(3H)-one (B25; 60 mg, 0.165 mmol) as a solid. LCMS (ES, m/z): 364.1 [M+H]⁺.

### Synthesis of Compound 157

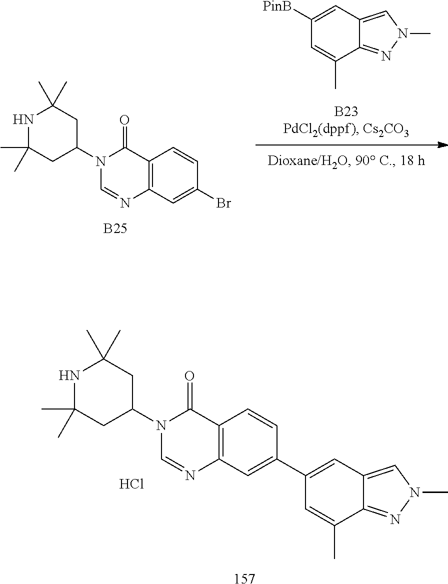

A mixture of 7-bromo-3-(2,2,6,6-tetramethylpiperidin-4-yl)quinazolin-4(3H)-one (B25; 60 mg, 0.17 mmol), 2,7-dimethyl-5-(4,4,5,5-tetramethyl-1,3,2-dioxaborolan-2-yl)-2H-indazole (B23; 50 mg, 0.18 mmol), PdCl₂(dppf) (14 mg, 0.017 mmol) and cesium carbonate (110 mg, 0.34 mmol) in dioxane (2.2 mL) and H₂O (0.2 mL) was heated to 90° C. for 16 h and then cooled to room temperature. The resulting mixture was filtered through Celite using ethyl acetate as an eluent, and the filtrate was concentrated under reduced pressure. The residue was purified by reverse phase chromatography using a C18 column eluting with acetonitrile in a 0.1% aqueous HCl solution (using a gradient of 5 to 70% acetonitrile). The fractions containing product were combined and lyophilized, to afford 7-(2,7-dimethyl-2H-indazol-5-yl)-3-(2,2,6,6-tetramethylpiperidin-4-yl)quinazolin-4(3H)-one hydrochloride (Compound 157; 19 mg, 0.041 mmol) as a solid. LCMS (ES, m/z): 430.3 [M+H]⁺. ¹H NMR (CH₃OH-d₄, 400 MHz): δ 9.26 (1H, s), 8.56 (1H, s), 8.41 (1H, d, J=8.4 Hz), 8.04-8.06 (2H, m), 7.99 (1H, s), 7.78 (1H, s), 7.65 (1H, s), 5.31 (1H, s), 4.34 (3H, s), 2.69 (3H, s), 2.65 (1H, s), 2.46 (2H, t, J=13.0 Hz), 2.21 (2H, d, J=13.3 Hz), 1.65 (6H, s), 1.59 (6H, s).

### Example 6

### Synthesis of Compound 158

### Synthesis of Intermediate B26

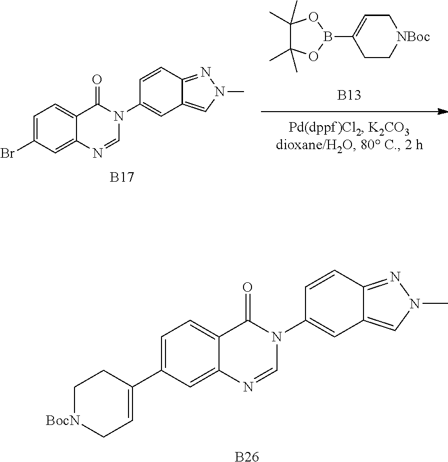

A mixture of 7-bromo-3-(2-methyl-2H-indazol-5-yl)quinazolin-4(3H)-one (B17 from Example 3; 300 mg, 0.85 mmol), tert-butyl 4-(4,4,5,5-tetramethyl-1,3,2-dioxaborolan-2-yl)-3,6-dihydropyridine-1(2H)-carboxylate (B13; 261 mg, 0.85 mmol), Pd(dppf)Cl₂ (20 mg, 0.09 mmol) and potassium carbonate (350 mg, 2.54 mmol) in dioxane (10 mL) and H₂O (2 mL) was heated to 80° C. for 2 h and then cooled to room temperature. The mixture was diluted with ethyl acetate (100 mL) and washed with saturated aqueous sodium bicarbonate (50 mL) and brine (50 mL). The organic layer was separated, dried over magnesium sulfate, filtered and concentrated under reduced pressure. The crude product was purified by silica gel column chromatography eluting with methanol in dichloromethane (0 to 10%), to afford tert-butyl 4-(3-(2-methyl-2H-indazol-5-yl)-4-oxo-3,4-dihydroquinazolin-7-yl) -3,6-dihydropyridine-1(2H)-carboxylate (B26; 260 mg) as a solid. LCMS (ES, m/z): 458.2 [M+H]⁺.

### Synthesis of Compound 158

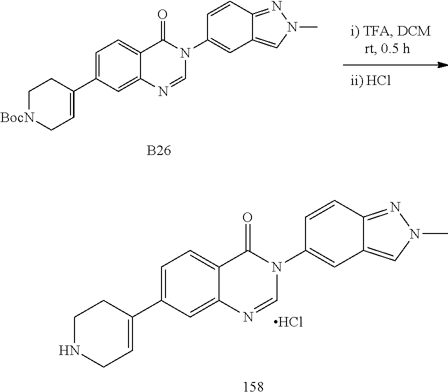

Trifluoroacetic acid (3 mL, 39 mmol) was added to a solution of tert-butyl 4-(3-(2-methyl-2H-indazol-5-yl)-4-oxo-3,4-dihydroquinazolin-7-yl)-3,6-dihydropyridine-1(2H)-carboxylate (B26; 92 mg, 0.2 mmol) in dichloromethane (3 mL), and the reaction mixture was stirred at room temperature for 0.5 h. The volatiles were removed under reduced pressure and the crude product was purified by reverse phase chromatography eluting with acetonitrile in a 0.1% aqueous HCl solution (using a gradient of 5 to 50% acetonitrile), to afford 3-(2-methyl-2H-indazol-5-yl)-7-(1,2,3,6-tetrahydropyridin-4-yl)quinazolin-4(3H)-one (Compound 158; 33 mg) as an HCl salt. LCMS (ES, m/z): 358.2 [M+H]⁺. ¹H NMR (DMSO-d₆, 400 MHz): δ_{H} 9.02 (2H, s), 8.49 (1H, s), 8.41 (1H, s), 8.19 (1H, d, J=8.3 Hz), 7.88 (1H, s), 7.69-7.76 (3H, m), 7.31 (1H, d, J=9.2 Hz), 6.52 (1H, s), 4.21 (3H, s), 3.36 (2H, s), 2.80 (2H, s). Note: Signal of two hydrogen atoms are overlapping with residual water peak from the deuterated solvent.

### Example 7

### Synthesis of Compound 159

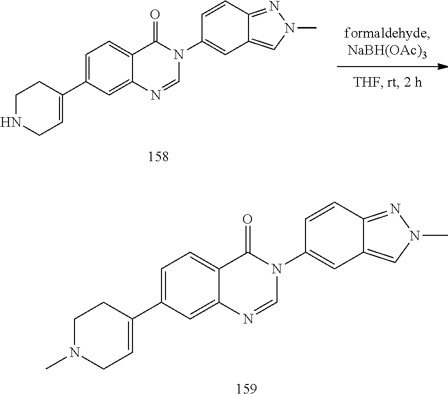

A mixture of 3-(2-methyl-2H-indazol-5-yl)-7-(1,2,3,6-tetrahydropyridin-4-yl)quinazolin-4(3H)-one (Compound 158 from Example 9; 100 mg, 0.25 mmol) and formaldehyde (37% in water, 103 mg, 0.085 mL, 1.27 mmol) was stirred at room temperature for 1 h. Sodium triacetoxyborohydride was then added to the mixture and stirred at room temperature for an additional 1 h. The mixture was then diluted with ethyl acetate (50 mL) and washed with saturated aqueous sodium bicarbonate (2×50 mL) and brine (50 mL). The organic layer was separated, dried over magnesium sulfate, filtered and concentrated under reduced pressure. The crude product was purified by silica gel column chromatography eluting with methanol in dichloromethane (1 to 10%) to afford 7-(1-methyl-1,2,3,6-tetrahydropyridin-4-yl)-3-(2-methyl-2H-indazol-5-yl)quinazolin-4(3H)-one (Compound 159; 23 mg) as a solid. LCMS (ES, m/z): 372.2 [M+H]⁺. ¹H NMR (CH₃OH-d₄:CDCl₃ (9:1), 400 MHz): δ_{H} 8.29 (2H, d, J=3.1 Hz), 8.24 (1H, d, J=8.4 Hz), 7.69-7.79 (4H, m), 7.33 (1H, d, J=9.1 Hz), 6.42 (1H, s), 4.26 (3H, s), 3.23 (2H, s), 2.78 (2H, d, J=5.9 Hz), 2.72 (2H, s), 2.43 (3H, s).

### Example 8

### Synthesis of Compound 160

### Synthesis of Intermediate B28

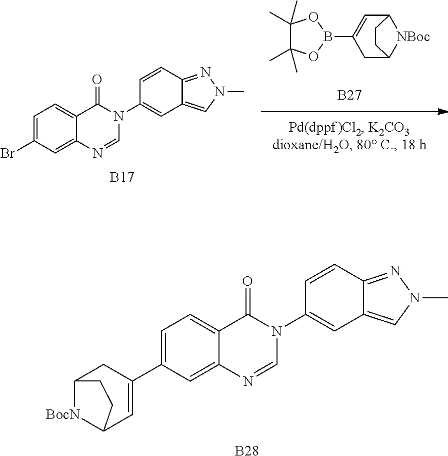

A mixture of 7-bromo-3-(2-methyl-2H-indazol-5-yl)quinazolin-4(3H)-one (B17 from Example 3; 200 mg, 0.56 mmol), tert-butyl 3-(4,4,5,5-tetramethyl-1,3,2-dioxaborolan-2-yl)-8-azabicyclo[3.2.1]oct-2-ene-8-carboxylate (B27; 189 mg, 0.56 mmol), Pd(dppf)Cl₂ (40 mg, 0.06 mmol) and potassium carbonate (234 mg, 1.69 mmol) in dioxane (50 mL) and H₂O (1 mL) was stirred at 80° C. for 18 h, and then cooled to room temperature. The mixture was diluted with ethyl acetate (50 mL) and washed with saturated sodium bicarbonate (25 mL) and brine (25 mL). The organic layer was then separated, dried over magnesium sulfate, filtered, and concentrated under reduced pressure, to afford tert-butyl 3-(3-(2-methyl-2H-indazol-5-yl)-4-oxo-3,4-dihydroquinazolin-7-yl)-8-azabicyclo[3.2.1]oct-2-ene-8-carboxylate (B28; 260 mg) as a solid. LCMS (ES, m/z): 484.2 [M+H]⁺.

### Synthesis of Compound 160

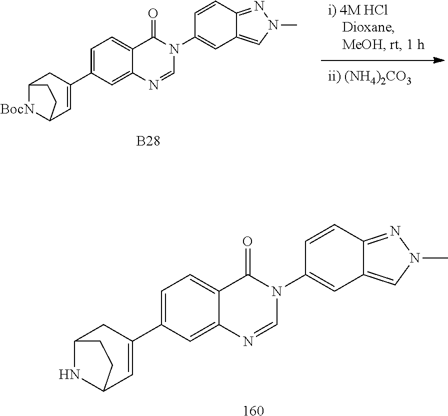 [SMILES: Cn1cc2cc(-n3cnc4cc(C5=CC6CCC(C5)N6C(=O)OC(C)(C)C)ccc4c3=O)ccc2n1 ; "B28" ; Cn1cc2cc(-n3cnc4cc(C5=CC6CCC(C5)N6)ccc4c3=O)ccc2n1 ; "160"]

A 4M solution of HCl in dioxane (4 mL) was added to a solution of tert-butyl 3-(3-(2-methyl-2H-indazol-5-yl)-4-oxo-3,4-dihydroquinazolin-7-yl)-8-azabicyclo[3.2.1]oct-2-ene-8-carboxylate (B28; 92 mg, 0.2 mmol) in methanol (2 mL), and the resulting mixture was stirred at room temperature for 1 h. The volatiles were then removed under reduced pressure, and the residue was purified by reverse phase chromatography eluting with acetonitrile in a 0.1% aqueous formic acid solution (using a gradient of 5 to 50% acetonitrile) to provide a solid, which was then dissolved in water (3 mL), neutralized with ammonium carbonate (20 mg, 0.19 mmol) and lyophilized, to afford 7-(8-azabicyclo[3.2.1]oct-2-en-3-yl)-3-(2-methyl-2H-indazol-5-yl)quinazolin-4(3H)-one (Compound 160; 11 mg). LCMS (ES, m/z): 384.1 [M+H]⁺. ¹H NMR (DMSO-d₆, 400 MHz): δ_{H} 8.48 (1H, s), 8.39 (1H, s), 8.13 (1H, d, J=8.3 Hz), 7.87 (1H, d, J=1.9 Hz), 7.68-7.71 (3H, m), 7.30 (1H, dd, J=9.1, 2.0 Hz), 6.79 (1H, d, J=5.6 Hz), 4.21 (3H, s), 4.01-4.08 (2H, m), 2.97 (1H, dd, J=17.3, 4.4 Hz), 2.01-2.08 (2H, m), 1.89-1.96 (1H, m), 1.71-1.77 (1H, m). Note: Signal of two hydrogen atoms are overlapping with solvent peak of residual DMSO-d₅.

### Example 9

### Synthesis of Compound 161

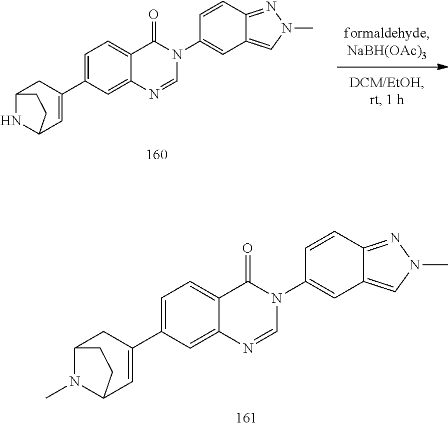

A mixture of 7-(8-azabicyclo[3.2.1]oct-2-en-3-yl)-3-(2-methyl-2H-indazol-5-yl)quinazolin-4(3H)-one (Compound 160 from Example 11; 50 mg, 0.12 mmol) and formaldehyde (37% in water, 0.05 mL, 0.60 mmol) in dichloromethane (6 mL) and ethanol (2 mL), was stirred at room temperature for 1 h. Sodium triacetoxyborohydride (151 mg, 0.71 mmol) was then added to the mixture and stirred for an additional 1 h. The mixture was diluted with ethyl acetate (50 mL), and washed with saturated aqueous sodium bicarbonate (2×50 mL) and brine (50 mL). The organic layer was separated, dried over magnesium sulfate, filtered, and concentrated under reduced pressure. The crude product was purified by reverse phase chromatography eluting with acetonitrile in a 0.1% aqueous HCl solution (using a gradient of 5 to 50% acetonitrile) to provide a solid that was dissolved in water (3 mL), neutralized with ammonium carbonate (20 mg, 0.19 mmol) and lyophilized. The resulting solid was then washed with water (2×1 mL), filtered, and dried, to afford 3-(2-methyl-2H-indazol-5-yl)-7-(8-methyl-8-azabicyclo[3.2.1]oct-2-en-3-yl)quinazolin-4(3H)-one (Compound 161; 14 mg) as a solid. LCMS (ES, m/z): 398.2 [M+H]⁺. ¹H NMR (DMSO-d₆, 400 MHz): δ_{H} 8.49 (1H, s), 8.42 (1H, s), 8.18 (1H, d, J=8.3 Hz), 7.88 (1H, s), 7.69-7.79 (3H, m), 7.30 (1H, dd, J=9.1, 1.9 Hz), 6.81 (1H, d, J=5.7 Hz), 4.27-4.30 (1H, m), 4.21 (3H, s), 4.07-4.17 (1H, m), 3.09-3.24 (1H, m), 2.72-2.86 (5H, m), 2.18-2.39 (2H, m), 1.93-2.00 (1H, m).

### Example 10

### Synthesis of Compound 162

### Synthesis of Intermediate B30

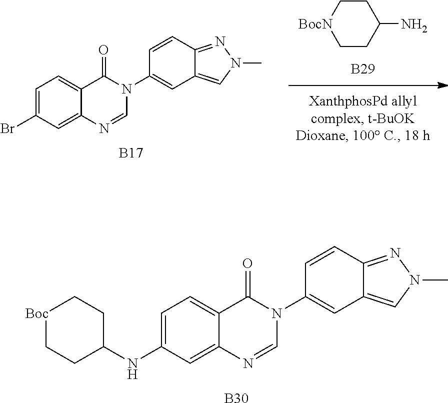

A mixture of 7-bromo-3-(2-methyl-2H-indazol-5-yl)quinazolin-4(3H)-one (B17 from Example 3; 50 mg, 0.14 mmol), tert-butyl 4-aminopiperazine-1-carboxylate (B29; 56 mg, 0.28 mmol), Xantphos-Pd-Allyl complex (11 mg, 0.014 mmol) and potassium tert-butoxide (24 mg, 0.21 mmol) in dioxane (5 mL) was heated to 100° C. for 24 h and then cooled to room temperature and diluted with dichloromethane. The resulting mixture was filtered through Celite and concentrated under reduced pressure to afford crude tert-butyl 4-((3-(2-methyl-2H-indazol-5-yl)-4-oxo-3,4-dihydroquinazolin-7-yl)amino)piperidine-1-carboxylate (B30; 64 mg) which was used in the next step without further purification. LCMS (ES, m/z): 475.3 [M+H]⁺.

### Synthesis of Compound 162

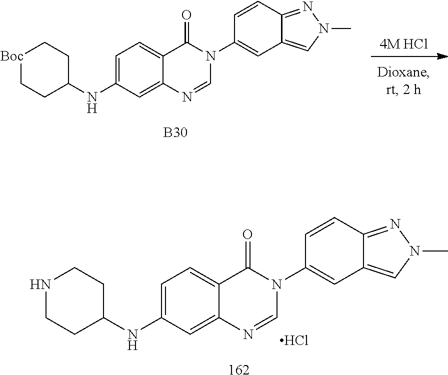

A mixture of tert-butyl 4-((3-(2-methyl -2H-indazol-5-yl)-4-oxo-3,4-dihydroquinazolin-7-yl)amino)piperidine-1-carboxylate (B30; 64 mg, 0.14 mmol) and 4M HCl in dioxane (3 mL) was stirred at room temperature for 2 h. The volatiles were then removed under reduced pressure and the crude product was purified by reverse phase chromatography eluting with acetonitrile in a 0.1% aqueous HCl solution (using a gradient of 5 to 50% acetonitrile), to afford 3-(2-methyl-2H-indazol-5-yl)-7-(piperazin-1-ylamino)quinazolin-4(3H)-one (Compound 162; 22 mg) as an HCl salt. LCMS (ES, m/z): 375.2 [M+H]⁺. ¹H NMR (CH₃OH-d₄, 400 MHz): δ_{H} 8.77 (1H, s), 8.36 (1H, s), 8.05 (1H, d, J=9.0 Hz), 7.86 (2H, s), 7.76 (1H, d, J=9.2 Hz), 7.33-7.36 (1H, m), 6.98-7.01 (1H, m), 6.78 (1H, s), 4.27 (3H, s), 3.82-3.87 (1H, m), 3.46-3.51 (2H, m), 3.17-3.24 (2H, m), 2.28-2.34 (2H, m), 1.75-1.82 (2H, m). Note: Signals for hydrogen atoms of HCl salt exchanged with the residual water from the CH₃OH-d₄.

### Example 11

### Synthesis of Compound 163

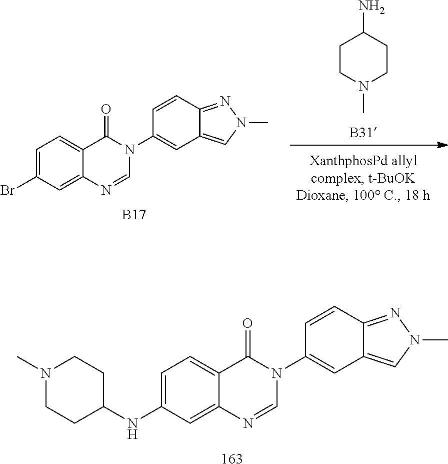

A mixture of 7-bromo-3-(2-methyl-2H-indazol-5-yl)quinazolin-4(3H)-one (B17 from Example 3; 300 mg, 0.85 mmol), 1-methylpiperidin-4-amine (B31′; 145 mg, 1.27 mmol), Xantphos-Pd-Allyl complex (32 mg, 0.042 mmol) and potassium tert-butoxide (142 mg, 1.27 mmol) in dioxane (15 mL) was stirred at 100° C. for 18 h and then cooled to room temperature and diluted with dichloromethane. The mixture was filtered through Celite and concentrated under reduced pressure, and purified by reverse phase chromatography eluting with acetonitrile in a 0.1% aqueous formic acid solution (using a gradient of 5 to 50% acetonitrile) to provide a solid that was dissolved in water (3 mL), neutralized with ammonium carbonate (20 mg, 0.19 mmol) and lyophilized, to afford 3-(2-methyl-2H-indazol-5-yl)-7-((1-methylpiperidin-4-yl)amino)quinazolin-4(3H)-one (Compound 163; 27 mg). LCMS (ES, m/z): 389.2 [M+H]⁺. ¹H NMR (DMSO-d₆, 400 MHz): δ_{H} 8.45 (1H, s), 8.17 (1H, s), 7.83 (1H, d, J=8.9 Hz), 7.79 (1H, s), 7.66 (1H, d, J=9.3 Hz), 7.24 (1H, d, J=9.2 Hz), 6.83 (1H, d, J=8.9 Hz), 6.63-6.65 (2H, m), 4.20 (3H, s), 2.76 (2H, d, J=10.6 Hz), 2.19 (3H, s), 2.08 (2H, t, J=11.2 Hz), 1.92 (2H, d, J=12.3 Hz), 1.40-1.49 (2H, m). Note: Signal of one hydrogen atom is overlapping with the residual water from DMSO-d₆.

### Example 12

### Synthesis of Compound 165

### Synthesis of Intermediate B33

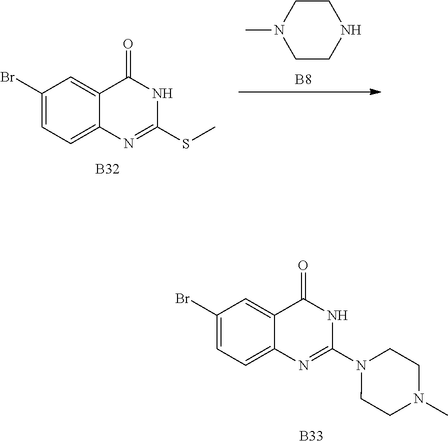

A mixture of 6-bromo-2-(methylthio)quinazolin-4(3H)-one (B32; 114 mg, 0.42 mmol) and N-methylpiperazine (B8; 2 mL) was stirred at 130° C. for 24 h, then cooled. The mixture was then suspended in diethyl ether and stirred for 1 h. The solid was collected by filtration and rinsed with diethyl ether, and then dissolved in dichloromethane/methanol and purified by silica gel column chromatography eluting with methanol in dichloromethane (1 to 20%), to afford 6-bromo-2-(4-methylpiperazin-1-yl)quinazolin-4(3H)-one (B33; 38 mg) as a solid. LCMS (ES, m/z): 323.1 [M+H]⁺. ¹H NMR (DMSO-d₆, 400 MHz): δ_{H} 11.52 (1H, br s), 7.95 (1H, d, J=2.3 Hz), 7.70 (1H, dd, J=8.7, 2.4 Hz), 7.21 (1H, s), 3.61 (4H, s), 2.36 (4H, s), 2.19 (3H, s). Note: B32 was prepared according to the procedure outlined in Erb. B., et al, J Heterocyclic Chem. 2000, 37(2), 253-260.

### Synthesis of Compound 165

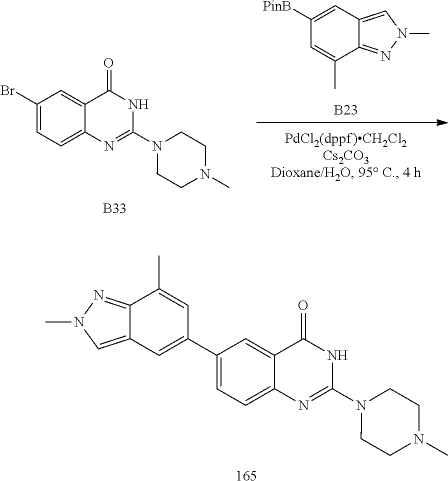

A mixture of 6-bromo-2-(4-methylpiperazin-1-yl)quinazolin-4(3H)-one (B33; 38 mg, 0.12 mmol), 2,7-dimethyl-5-(4,4,5,5-tetramethyl-1,3,2-dioxaborolan-2-yl)-2H-indazole (B23; 35.2 mg, 0.13 mmol), cesium carbonate (76 mg, 0.23 mmol) and PdCl₂(dppf)-CH₂Cl₂ (9.6 mg, 0.01 mmol) in dioxane (800 μL) and water (40 μL), in a capped vial, was purged with argon for 10 min and then stirred at 95° C. for 4 h. The mixture was then cooled, dimethylformamide was added, and the mixture was filtered through Celite and rinsed with dimethylformamide. The filtrate was then concentrated and purified by silica gel column chromatography eluting with methanol in dichloromethane (5 to 20%). The recovered material was suspended in ethyl acetate and stirred for 30 min at 0° C., and the solid was collected by filtration and rinsed with cold ethyl acetate, to afford 6-(2,7-dimethyl-2H-indazol-5-yl)-2-(4-methylpiperazin-1-yl)quinazolin-4(3H)-one (Compound 165; 25 mg) as a solid. LCMS (ES, m/z): 389.2 [M+H]⁺. ¹H NMR (DMSO-d₆, 400 MHz): δ_{H} 11.42 (1H, s), 8.33 (1H, s), 8.16 (1H, s), 7.95 (1H, d, J=8.5 Hz), 7.80 (1H, s), 7.38 (1H, s), 7.34 (1H, d, J=8.5 Hz), 4.18 (3H, s), 3.62 (4H, br s), 2.56 (3H, s), 2.38 (4H, br s), 2.20 (3H, s).

### Example 13

### Synthesis of Compound 166

### Synthesis of Intermediate B34

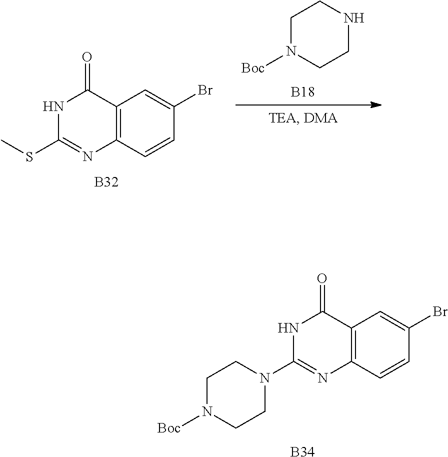

tert-Butyl piperazine-1-carboxylate (258 mg, 1.39 mmol) and triethylamine (0.19 mL, 1.4 mmol) were added to a solution of 6-bromo-2-(methylthio)quinazolin-4(3H)-one (B32 from Example 16; 188 mg, 0.64 mmol) in dimethylacetamide (1.5 mL), and the reaction mixture was heated to 120° C. for 5 days. The mixture was then concentrated, suspended on silica gel, and purified by normal phase chromatography on a Redisep Gold column (12 g), eluting with methanol (0-4%) in dichloromethane, to afford tert-butyl 4-(6-bromo-4-oxo-3,4-dihydroquinazolin-2-yl)piperazine-1-carboxylate as a solid (B34; 78 mg). LCMS (ES, m/z): 409.2 [M+H]⁺.

### Synthesis of Intermediate B35

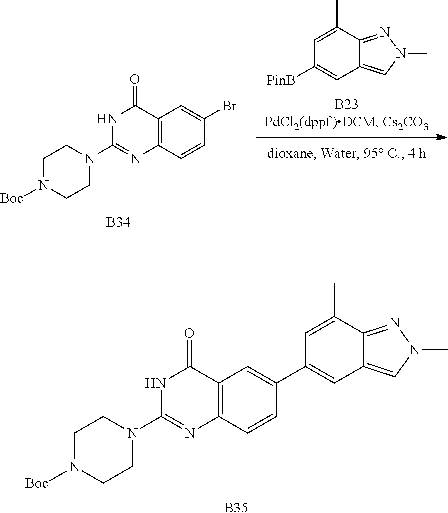

A mixture of tert-butyl 4-(6-bromo-4-oxo-3,4-dihydroquinazolin-2-yl)piperazine-1-carboxylate (B34; 84 mg, 0.21 mmol), 2,7-dimethyl-5-(4,4,5,5-tetramethyl-1,3,2-dioxaborolan-2-yl)-2H-indazole (B23; 67 mg, 0.25 mmol), dioxane (2.1 mL), water (0.1 mL), cesium carbonate (168 mg, 0.52 mmol) and PdCl₂(dppf)·DCM (16.8 mg, 0.02 mmol) was purged with argon for 10 min, and then heated to 95° C. for 4 h. The reaction mixture was then cooled to room temperature, dimethylformamide was added, and the pH was adjusted to 7 using 1N hydrochloric acid. The mixture was then filtered through Celite®, rinsed with dimethylformamide, and the filtrate was concentrated. The crude material was suspended on silica gel and purified on a Redisep Gold column (12 g) eluting with methanol (2-6%) in dichloromethane. The recovered material was then stirred in ethyl acetate for 30 min, cooled to 0° C., and collected by vacuum filtration. The solid was rinsed with cold ethyl acetate, to afford tert-butyl 4-(6-(2,7-dimethyl-2H-indazol-5-yl)-4-oxo-3,4-dihydroquinazolin-2-yl)piperazine-1-carboxylate (B35; 70.7 mg). LCMS (ES, m/z): 475.2 [M+H]⁺. ¹H NMR (DMSO-d₆, 400 MHz): δ_{H} 11.50 (1H, s), 8.33 (1H, s), 8.17 (1H, s), 7.96 (1H, d, J=8.5 Hz), 7.81 (1H, s), 7.36-7.38 (2H, m), 4.18 (3H, s), 3.62 (4H, s), 3.14 (4H, s), 2.56 (3H, s), 1.42 (9H, s).

### Synthesis of Compound 166

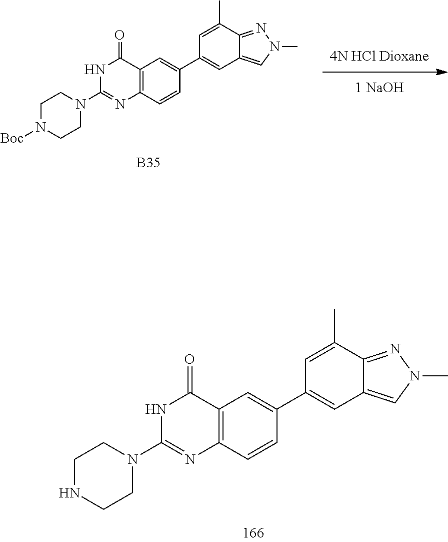 [SMILES: "B35" ; Cc1cc(-c2ccc3nc(N4CCN(C(=O)OC(C)(C)C)CC4)[nH]c(=O)c3c2)cc2cn(C)nc12 ; Cc1cc(-c2ccc3nc(N4CCNCC4)[nH]c(=O)c3c2)cc2cn(C)nc12]

Hydrochloric acid in dioxane (4N, 2 mL, 78 mmol) was added to tert-butyl 4-(6-(2,7-dimethyl-2H-indazol-5-yl)-4-oxo-3,4-dihydroquinazolin-2-yl)piperazine-1-carboxylate (B36; 68 mg, 0.41 mmol), and the mixture was stirred for 24 h at room temperature, then concentrated to dryness. The material was then added to water and the pH was adjusted to 6 using 1N sodium hydroxide. The resulting solid was stirred for 2 h, collected by filtration, rinsed with cold water, and dried to afford 6-(2,7-dimethyl-2H-indazol-5-yl)-2-(piperazin-1-yl)quinazolin-4(3H)-one (Compound 166; 22 mg). LCMS (ES, m/z): 375.2 [M+H]⁺. ¹H NMR (DMSO-d₆, 400 MHz): δ_{H} 8.33 (1H, s), 8.15 (1H, s), 7.94 (1H, d,=7.2 Hz), 7.80 (1H, s), 7.38 (1H, s), 7.34 (1H, m), 4.18 (3H, s), 3.58 (4H, s), 2.79 (4H, s), 2.56 (3H, s).

### Example 14

### Synthesis of Compound 167

### Synthesis of Intermediate B36

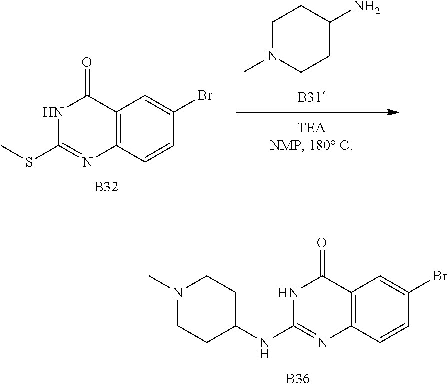

Triethylamine (0.5 mL, 0.35 mmol) was added to a mixture of 6-bromo-2-(methylthio)quinazolin-4(3H)-one (B32 from Example 16; 240 mg, 0.89 mmol) and 1-methylpiperidin-4-amine (B31′; 0.5 mL, 0.35 mmol) in N-methyl-2-pyrrolidone (1.8 mL), and the resulting mixture was heated to 180° C. for 12 h. The reaction mixture was then cooled to room temperature, water was added, and the solid was collected by vacuum filtration. The crude material was then dried and suspended on silica gel, and purified by column chromatography on a Redisep column (12 g) eluting with methanol (7.5 to 30%) in dichloromethane, to afford (B36; 50 mg). LCMS (ES, m/z): 336.9 [M+H]⁺.

### Synthesis of Compound 167

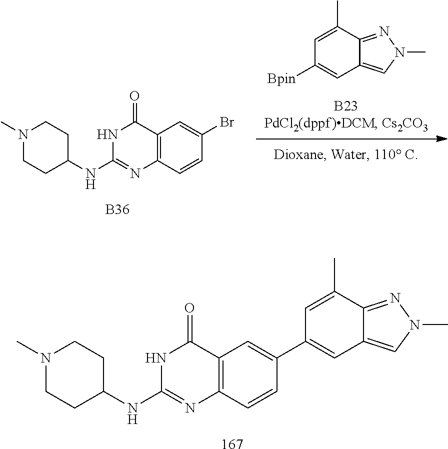

Argon was bubbled through a suspension of 6-bromo-2-((1-methylpiperidin-4-yl)amino)quinazolin-4(3H)-one (B36; 48 mg, 0.14 mmol), 2,7-dimethyl-5-(4,4,5,5-tetramethyl-1,3,2-dioxaborolan-2-yl)-2H-indazole (B23; 46 mg, 0.17 mmol), cesium carbonate (115 mg, 0.35 mmol) and Pd(dppf)·DCM (11.5 mg, 0.014 mmol) in dioxane (1.4 mL) and water (0.07 mL) for 10 min, and the mixture was then heated tot 110° C. and stirred for 20 h. The reaction mixture was then cooled to room temperature, dimethylformamide was added, and the pH of the mixture was adjusted to 7 using 1N hydrochloric acid. The suspension was filtered through Celite®, rinsed with dimethylformamide, and the filtrate was concentrated. The recovered material was purified by chromatography on a Redisep C-18 column (15.5 g) eluting with 0.1% trifluoroacetic acid in H₂O/0.1% trifluoroacetic acid in acetonitrile (using a gradient of 0 to 50% acetonitrile). Selected fractions were then lyophilised, and the resulting solid was added to water, neutralized with 1N sodium hydroxide, and collected by filtration, to afford 642,7-dimethyl-2H-indazol-5-yl)-2-((1-methylpiperidin-4-yl)amino)quinazolin-4(3H)-one (Compound 167; 5.2 mg). LCMS (ES, m/z): 403.2 [M+H]⁺. ¹H NMR (DMSO-d₆, 400 MHz): δ_{H} 10.64 (1H, s), 8.32-8.32 (1H, m), 8.12 (1H, s), 7.91 (1H, dd,=8.6, 2.3 Hz), 7.77 (1H, s), 7.35 (1H, s), 7.31 (1H, d, J=8.5 Hz), 6.27 (1H, d, J=7.3 Hz), 4.17 (3H, s), 3.82 (1H, br s), 2.72 (2H, s), 2.55 (3H, s), 2.21 (3H, s), 2.15 (2H, br s), 1.94 (2H, d, J=12.3 Hz), 1.45-1.53 (2H, m).

### Example 15

### Synthesis of Compound 182

### Synthesis of Intermediate B41

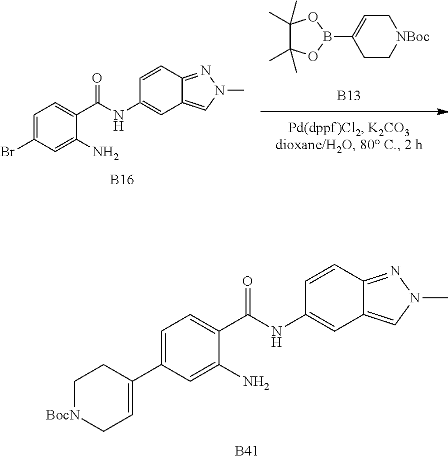

A mixture of 2-amino-4-bromo-N-(2-methyl-2H-indazol-5-yl)benzamide (B16 from Example 2; 1 g, 2.9 mmol), tert-butyl 4-(4,4,5,5-tetramethyl-1,3,2-dioxaborolan-2-yl)-3,6-dihydropyridine-1(2H)-carboxylate (B13; 895 mg, 2.9 mmol), Pd(dppf)Cl₂ (100 mg, 0.14 mmol) and potassium carbonate (1.2 g, 8.69 mmol) in dioxane (20 mL) and H₂O (4 mL) was heated to 80° C. for 2 h and then cooled to room temperature. The mixture was diluted with ethyl acetate (100 mL) and washed with saturated sodium bicarbonate (50 mL) and brine (50 mL). The organic layer was separated, dried over magnesium sulfate, filtered, and concentrated under reduced pressure. The crude product was purified by normal phase chromatography eluting with methanol (0 to 10%) in dichloromethane, to afford tert-butyl 4-(3-amino-4-((2-methyl-2H-indazol-5-yl)carbamoyl)phenyl)-3,6-dihydropyridine-1(2H)-carboxylate (B41; 260 mg) as a solid. LCMS (ES, m/z): 448.3 [M+H]⁺.

### Synthesis of Intermediate B42

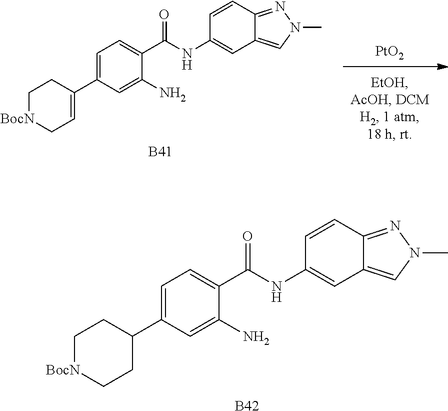

tert-Butyl 4-(3-amino-4-((2-methyl-2H-indazol-5-yl)carbamoyl)phenyl)-3,6-dihydropyridine-1(2H)-carboxylate (B41; 120 mg, 0.27 mmol) was dissolved in a mixture of ethanol (5 mL) and dichloromethane (2 mL), and acetic acid (0.5 mL) and platinum dioxide (20 mg, 0.09 mmol) were added. The resulting mixture was stirred under hydrogen (1 atm) for 18 h. Celite (100 mg) was then added, and the mixture was filtered through Celite and washed with dichloromethane (10 mL). The filtrate was then concentrated under reduced pressure, to afford tert-butyl 4-(3-amino-4-((2-methyl-2H-indazol-5-yl)carbamoyl)phenyl)piperidine-1-carboxylate (B42; 115 mg) as a solid. LCMS (ES, m/z): 450.3 [M+H]⁺.

### Synthesis of Compound 182

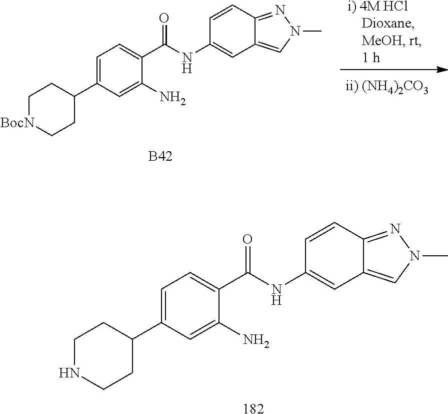

A 4M solution of hydrochloric acid in dioxane (4 mL) was added to a solution of tert-butyl 4-(3-amino-4-((2-methyl-2H-indazol-5-yl)carbamoyl)phenyl)piperidine-1-carboxylate (B42; 115 mg, 0.26 mmol) in methanol (2 mL), and the reaction mixture was stirred at room temperature for 1 h. The volatiles were then removed under reduced pressure to afford a solid, which was purified by reverse phase chromatography eluting with acetonitrile (5 to 50%) in 0.1% aqueous formic acid. The purified solid was then dissolved in water (3 mL), neutralized with ammonium carbonate (20 mg, 0.19 mmol), and lyophilized, to afford 2-amino-N-(2-methyl-2H-indazol-5-yl)-4-(piperidin-4-yl)benzamide (Compound 182; 29 mg) as a solid. LCMS (ES, m/z): 350.2 [M+H]⁺. ¹H NMR (DMSO-d₆, 400 MHz): δ_{H} 9.86 (1H, s), 8.35 (1H, s), 8.24 (1H, s), 8.11 (1H, s), 7.57 (1H, d,=8.2 Hz), 7.52 (1H, d,=9.3 Hz), 7.40 (1H, d,=9.3 Hz), 6.58 (1H, s), 6.45 (1H, d, J=8.2 Hz), 6.32 (2H, s), 4.12 (3H, s), 3.17 (2H, d, J=12.4 Hz), 2.75 (2H, t, J=12.1 Hz), 2.58 (1H, t, J=11.4 Hz), 1.76 (2H, d, J=12.9 Hz), 1.56-1.65 (2H, m).

### Example 16

### Synthesis of Compound 172

### Synthesis of Intermediate B43

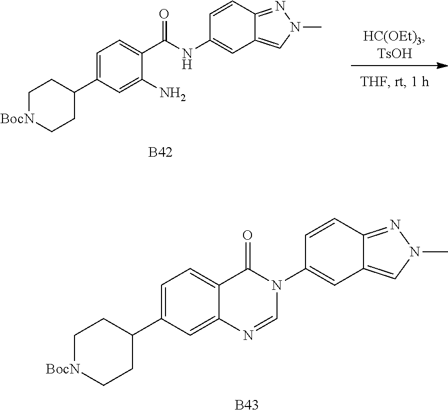

Triethyl orthoformate (1.2 g, 8.1 mmol) and p-toluenesulfonic acid (15 mg, 0.08 mmol) were added to a solution of tert-butyl 4-(3-amino-4-((2-methyl-2H-indazol-5-yl)carbamoyl)phenyl)piperidine-1-carboxylate (B42 from Example 24; 364 mg, 0.81 mmol) in tetrahydrofuran (5 mL), and the reaction mixture was stirred at room temperature for 1 h. Ethyl acetate (50 mL) was then added, and the mixture was washed with saturated sodium bicarbonate (2×50 mL) and brine (50 mL), dried over magnesium sulfate, filtered and concentrated under reduced pressure, to afford tert-butyl 4-(3-(2-methyl -2H-indazol-5-yl)-4-oxo-3,4-dihydroquinazolin-7-yl)piperidine-1-carboxylate (B43; 364 mg) as a solid. LCMS (ES, m/z): 460.3 [M+H]⁺.

### Synthesis of Compound 172

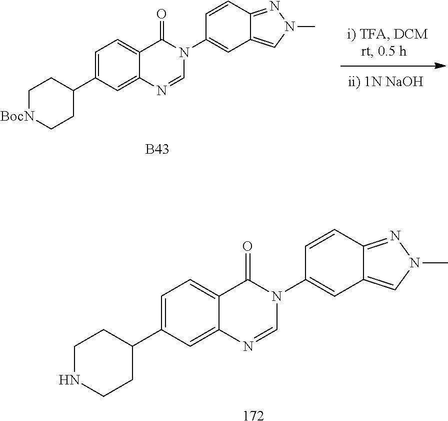

Trifluoroacetic acid (3 mL, 39 mmol) was added to a solution of tert-butyl 4-(3-(2-methyl-2H-indazol-5-yl)-4-oxo-3,4-dihydroquinazolin-7-yl)piperidine-1-carboxylate (B43; 142 mg, 0.3 mmol) in dichloromethane (3 mL), and the reaction mixture was stirred at room temperature for 0.5 h. The volatiles were removed under reduced pressure, and the crude product was purified by reverse phase chromatography eluting with acetonitrile (5 to 50%) in 0.1% aqueous trifluoroacetic acid. A 25 mg portion of the resulting solid was then added to water (1 mL), and the pH was adjusted to 14 by the dropwise addition of 1M sodium hydroxide. The resulting solid was centrifuged, decanted, then added to water (1 mL), and sonicated for 30 seconds. This process was repeated three times, and the solid was then lyophilized, to afford 3-(2-methyl-2H-indazol-5-yl)-7-(piperidin-4-yl)quinazolin-4(3H)-one (Compound 172; 19 mg) as a solid. LCMS (ES, m/z): 360.1 [M+H]⁺. ¹H NMR (DMSO-d₆, 400 MHz): δ_{H} 8.47 (1H, s), 8.40 (0.3H***, bs), 8.35 (1H, s), 8.13 (1H, d, J=8.2 Hz), 7.85 (1H, s), 7.70 (1H, d, J=9.1 Hz), 7.55 (1H, s), 7.48 (1H, d, J=8.3 Hz), 7.28 (1H, d, J=9.1 Hz), 4.21 (3H, s), 3.11 (2H, d, J=12.3 Hz), 2.84 (1H, t, J=12.0 Hz), 2.69 (2H, t, J=12.4 Hz), 1.81 (2H, d, J=12.6 Hz), 1.59-1.68 (2H, m). ***Exchangeable NH proton

### Example 17

### Synthesis of Compound 173

### Synthesis of Intermediate B44

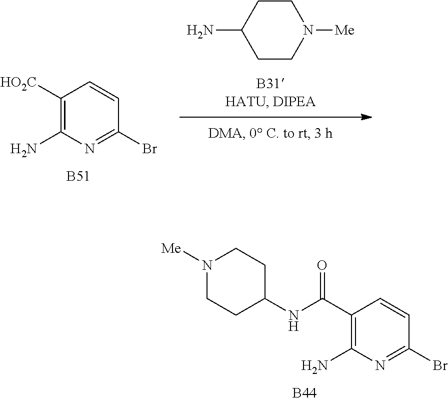

A mixture of 2-amino-6-bromonicotinic acid (B51; 100 mg, 0.46 mmol) and 4-amino-1-methylpiperidine (B31′; 60 mg, 0.53 mmol) in dimethylacetamide (2.3 mL) was cooled to 0° C., and treated with diisopropylethylamine (250 μL, 1.43 mmol) dropwise, followed by hexafluorophosphate azabenzotriazole tetramethyl uronium (194 mg, 0.51 mmol), and the mixture was warmed to room temperature and stirred for 3 h. The reaction mixture was then diluted with ethyl acetate (20 mL) and washed with saturated sodium bicarbonate (20 mL) and brine (20 mL). The organic layer was dried over anhydrous sodium sulfate and concentrated in vacuo, to afford 2-amino-6-bromo-N-(1-methylpiperidin-4-yl)nicotinamide (B44; 140 mg) as a solid. LCMS (ES, m/z): 313.1 [M+H]⁺.

### Synthesis of Intermediate B45

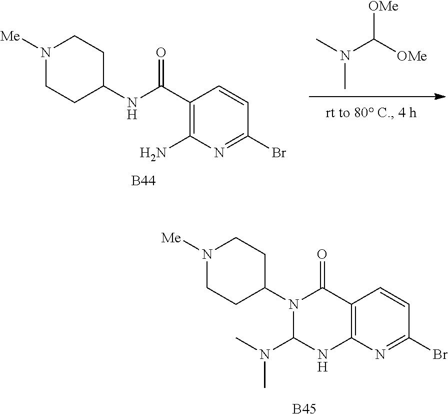

A mixture of 2-amino-6-bromo-N-(1-methylpiperidin-4-yl)nicotinamide (B44; 140 mg, 0.45 mmol) and N,N-dimethylformamide dimethyl acetal (1.2 mL, 9 mmol) in a 10 mL sealed tube was heated to 80° C. for 4 h. The reaction mixture was then dissolved in dichloromethane (20 mL) and washed with aqueous sodium hydroxide (20%; 15 mL), then brine (20 mL). The organic phase was dried over sodium sulfate and the residue concentrated in vacuo, to afford 7-bromo-2-(dimethylamino)-3-(1-methylpiperidin-4-yl)-2,3-dihydropyrido[2,3-d]pyrimidin-4(1H)-one (B45; 144 mg) as a solid. LCMS (ES, m/z): 368.1 [M+H]⁺.

### Synthesis of Compound 173

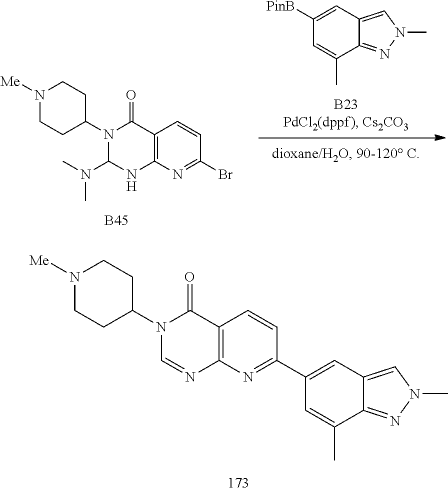

A mixture of 7-bromo-2-(dimethylamino)-3-(1-methylpiperidin-4-yl)-2,3-dihydropyrido[2,3-d]pyrimidin-4(1H)-one (B45; 88 mg, 0.24 mmol), 2,7-dimethyl-5-(4,4,5,5-tetramethyl-1,3,2-dioxaborolan-2-yl)-2H-indazole (B23; 72 mg, 0.27 mmol), PdCl₂(dppf) (20 mg, 0.024 mmol) and cesium carbonate (235 mg, 0.72 mmol) in dioxane (3.4 mL) and H₂O (0.3 mL) was heated to 90° C. for 16 h under an atmosphere of nitrogen, and then heated to 120° C. overnight. Next, the mixture was diluted with dimethylformamide and filtered through Celite, and the residue was concentrated under reduced pressure, then stirred in 1N hydrochloric acid (20 mL) for 15 minutes. The aqueous layer was extracted with dichloromethane (3×15 mL), and the aqueous phase was neutralized with ammonium carbonate and washed with dichloromethane (3×15 mL). The aqueous phase was then concentrated in vacuo, and the residue was purified by reverse phase flash chromatography on a C18 column (30 g) eluting with acetonitrile (0-70%, slow gradient) in 0.1% aqueous formic acid. Fractions containing the product were combined, neutralized with ammonium carbonate, and lyophilized. The resulting solid was triturated with methyl tert-butyl ether (3 mL), then ethyl acetate (3 mL), and traces of solvent were removed under reduced pressure, to afford 7-(2,7-dimethyl-2H-indazol-5-yl)-3-(1-methylpiperidin-4-yl)pyrido[2,3-d]pyrimidin-4(3H)-one (Compound 173; 27 mg) as a solid. LCMS (ES, m/z): 389.2 [M+H]⁺. ¹H NMR (CH₃OH-d₄, 400 MHz): δ 8.59-8.62 (2H, m), 8.44 (1H, s), 8.34 (1H, s), 8.13 (1H, d, J=8.5 Hz), 8.00 (1H, s), 4.71 (1H, s), 4.25 (3H, s), 3.12 (2H, d, J=11.2 Hz), 2.65 (3H, s), 2.41 (3H, s), 2.34 (2H, t, J=12.0 Hz), 2.24 (2H, t, J=12.7 Hz), 2.02 (2H, d, J=11.8 Hz).

### Example 18

### Synthesis of Compound 174

### Synthesis of Intermediate B46

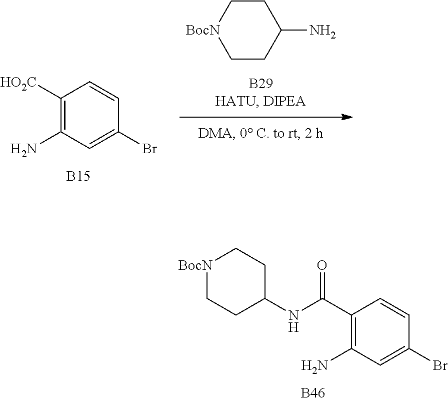 [SMILES: CC(C)(C)OC(=O)N1CCC(N)CC1 ; "B15" ; Nc1cc(Br)ccc1C(=O)O ; "B46" ; CC(C)(C)OC(=O)N1CCC(NC(=O)c2ccc(Br)cc2N)CC1]

A mixture of 2-amino-4-bromobenzoic acid (B15; 100 mg, 0.46 mmol) and 4-amino-1-Boc-piperidine (B29; 102 mg, 0.51 mmol) in dimethylacetamide (2.3 mL) was cooled to 0° C., and treated with diisopropylethylamine (250 μL, 1.431 mmol) dropwise, followed by hexafluorophosphate azabenzotriazole tetramethyl uronium (194 mg, 0.51 mmol), and the mixture was warmed to room temperature and stirred for 2 h. The reaction mixture was then diluted with ethyl acetate (20 mL) and washed with saturated aqueous ammonium chloride (20 mL), followed by saturated sodium bicarbonate (20 mL), and brine (20 mL). The organic layer was dried over anhydrous sodium sulfate and concentrated in vacuo, to afford tert-butyl 4-(2-amino-4-bromobenzamido)piperidine-1-carboxylate (B46; 178 mg) as a solid. LCMS (ES, m/z): 342.1 [M+H−^{t}Bu]⁺.

### Synthesis of Intermediate B47

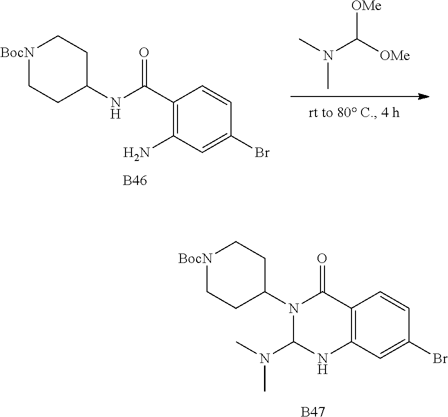

A mixture of tert-butyl 4-(2-amino-4-bromobenzamido)piperidine-1-carboxylate (B46; 70 mg, 0.18 mmol) and N,N-dimethylformamide dimethyl acetal (470 μL, 3.53 mmol) in a 10 mL sealed tube was heated to 80° C. for 4 h. The mixture was then dissolved in ethyl acetate (20 mL) and washed with saturated sodium bicarbonate (20 mL), then brine (2×20 mL). The organic phase was dried over sodium sulfate and concentrated in vacuo, to afford tert-butyl 4-(7-bromo-2-(dimethylamino)-4-oxo-1,4-dihydroquinazolin-3(2H)-yl)piperidine-1-carboxylate (B47; 75 mg) as a solid. LCMS (ES, m/z): 453.2 [M+H]⁺.

### Synthesis of Compound 174

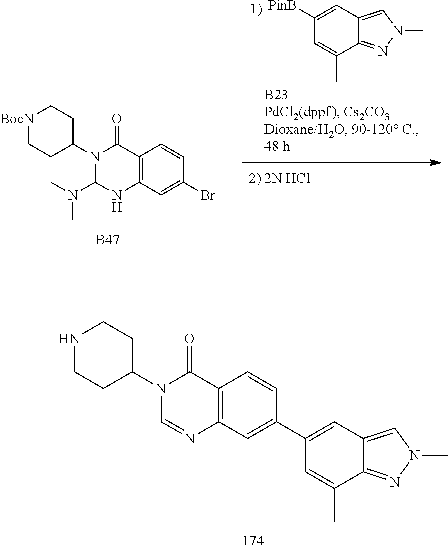

A mixture of tert-butyl 4-(7-bromo-2-(dimethylamino)-4-oxo-1,4-dihydroquinazolin-3(2H)-yl)piperidine-1-carboxylate (B47; 80 mg, 0.18 mmol), 2,7-dimethyl-5-(4,4,5,5-tetramethyl-1,3,2-dioxaborolan-2-yl)-2H-indazole (B23; 54 mg, 0.2 mmol), PdCl₂(dppf) (14 mg, 0.02 mmol) and cesium carbonate (175 mg, 0.54 mmol) in dioxane (2.5 mL) and H₂O (0.2 mL) was heated to 90° C. for 16 h, and then heated to 120° C. for 32 hours. Next, the reaction mixture was concentrated under reduced pressure, and the residue was purified by flash chromatography on a silica gel column (12 g) eluting with methanol (0-25%) in dichloromethane. The fractions containing the product were combined and evaporated under reduced pressure, and the resulting solid was stirred vigorously in 2N aqueous hydrochloric acid (15 mL) at room temperature for 6 hours. The resulting solution was washed with dichloromethane (2×15 mL) and concentrated in vacuo. The residue was purified by reverse phase flash chromatography on a C18 column (12 g) eluting with acetonitrile (5-70%) in 0.1% aqueous formic acid. The fractions containing the product were combined, neutralized with ammonium carbonate, and lyophilized. The resulting solid was triturated with methyl tert-butyl ether (3 mL) followed by ethyl acetate (3 mL). Traces of solvent were removed under reduced pressure, to afford 7-(2,7-dimethyl-2H-indazol-5-yl)-3-(piperidin-4-yl)quinazolin-4(3H)-one (Compound 174; 15 mg) as a solid. LCMS (ES, m/z): 374.2 [M+H]⁺. ¹H NMR (CH₃OH-d₄, 400 MHz): δ 8.40 (1H, s), 8.28-8.30 (2H, m), 7.88-7.92 (3H, m), 7.47 (1H, s), 4.25 (3H, s), 3.23 (2H, d, J=13.1 Hz), 2.79 (2H, t, J=12.0 Hz), 2.65 (3H, s), 1.96-2.01 (4H, m). (*Methine proton of the piperidine substituent obscured by water signal).

### Example 19

### Synthesis of Compound 175

### Synthesis of Intermediate B49

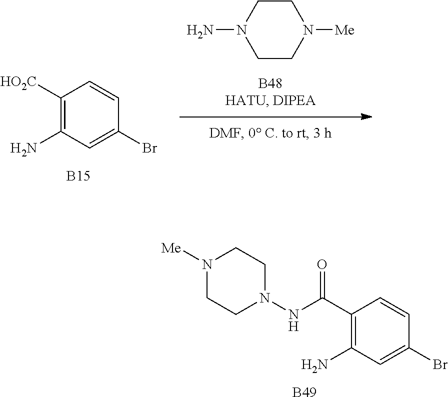

A mixture of 2-amino-4-bromobenzoic acid (B15; 150 mg, 0.69 mmol) and 1-amino-4-methylpiperazine (B48; 90 mg, 0.78 mmol) in dimethylformamide (3.5 mL) was cooled to 0° C. and treated with diisopropylethylamine (360 μL, 2 mmol) dropwise, followed by hexafluorophosphate azabenzotriazole tetramethyl uronium (290 mg, 0.76 mmol), and the mixture was warmed to room temperature and stirred for 3 h. Next, the mixture was diluted with ethyl acetate (20 mL) and washed with saturated aqueous ammonium chloride (20 mL), sodium bicarbonate (20 mL), then brine (20 mL). The organic layer was dried over anhydrous sodium sulfate and concentrated in vacuo, to afford 2-amino-4-bromo-N-(4-methylpiperazin-1-yl)benzamide (B49; 166 mg, 0.53 mmol) as a solid. LCMS (ES, m/z): 313.1 [M+H]⁺.

### Synthesis of Intermediate B50

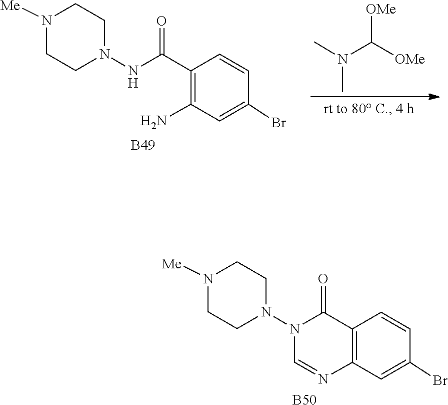

A mixture of 2-amino-4-bromo-N-(4-methylpiperazin-1-yl)benzamide (B49; 140 mg, 0.45 mmol) and N,N-dimethylformamide dimethyl acetal (1.8 mL, 13.5 mmol) in a 10 mL sealed tube was heated to 80° C. for 4 h. The reaction mixture was then dissolved in ethyl acetate (20 mL) and washed with sodium bicarbonate (15 mL), then brine (20 mL). The organic phase was dried over sodium sulfate and the residue concentrated in vacuo, to afford 7-bromo-3-(4-methylpiperazin-1-yl)quinazolin-4(3H)-one (B50; 110 mg) as a solid. LCMS (ES, m/z): 323.1 [M+H]⁺.

### Synthesis of Compound 175

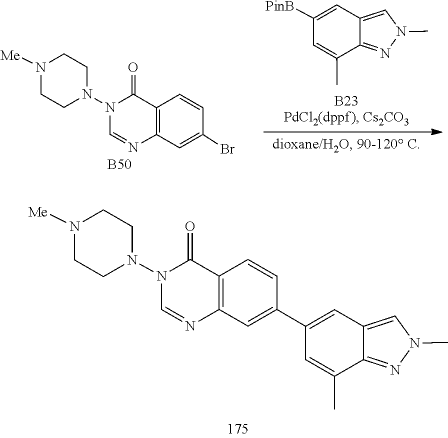

A mixture of 7-bromo-3-(4-methylpiperazin-1-yl)quinazolin-4(3H)-one (B50; 97 mg, 0.30 mmol), 2,7-dimethyl-5-(4,4,5,5-tetramethyl-1,3,2-dioxaborolan-2-yl)-2H-indazole (B23; 90 mg, 0.33 mmol), PdCl₂(dppf) (25 mg, 0.031 mmol) and cesium carbonate (196 mg, 0.60 mmol) in dioxane (4.3 mL) and H₂O (0.4 mL) was heated to 90° C. for 16 h under a nitrogen atmosphere, and then heated to 120° C. overnight. Next, the mixture was diluted with dimethylformamide and filtered through Celite, then concentrated under reduced pressure. The residue was then stirred for 15 min in 1N hydrochloric acid (20 mL). The aqueous layer was extracted with dichloromethane (3×15 mL), and the aqueous phase was filtered under vacuum, neutralized with sodium carbonate, and washed with dichloromethane (3×15 mL), and concentrated in vacuo. The residue was purified by reverse phase flash chromatography on a C18 column (12 g) eluting with acetonitrile (5-70%) in 0.1% aqueous formic acid. Fractions containing the product were combined, neutralized with ammonium carbonate, and lyophilized. The resulting solid was triturated with methyl tert-butyl ether (3 mL), then ethyl acetate (3 mL), and traces of solvent were removed under reduced pressure, to afford 7-(2,7-dimethyl-2H-indazol-5-yl)-3-(4-methylpiperazin-1-yl)quinazolin-4(3H)-one (Compound 175; 55 mg) as a solid. LCMS (ES, m/z): 389.2 [M+H]⁺. ¹H NMR (CH₃OH-d₄, 400 MHz): δ 8.31-8.26 (3H, m), 7.88-7.92 (3H, m), 7.47 (1H, s), 4.07-4.25 (5H, br), 3.18 (1H, br), 2.98 (2H, br) 2.65 (6H, br), 2.44 (3H, s).

### Example 20

### Synthesis of Compound 176

### Synthesis of Intermediate B52

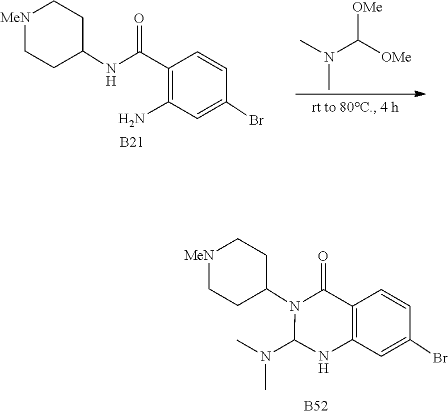

A mixture of tert-butyl 2-amino-4-bromo-N-(1-methylpiperidin-4-yl)benzamide (B21 from Example 6; 352 mg, 0.88 mmol) and N,N-dimethylformamide dimethyl acetal (2 mL, 15 mmol) in a 10 mL sealed tube was heated to 80° C. for 4 h. The reaction mixture was then dissolved in ethyl acetate (20 mL) and washed with saturated sodium bicarbonate (20 mL), then brine (2×20 mL). The organic phase was dried over sodium sulfate and concentrated in vacuo. The resulting solid was triturated with methyl tert-butyl ether (5 mL) and traces of solvent were removed under reduced pressure, to afford 7-bromo-2-(dimethylamino)-3-(1-methylpiperidin-4-yl)-2,3-dihydroquinazolin-4(1H)-one (B52; 201 mg, 0.55 mmol) as a solid. LCMS (ES, m/z): 367.1 [M+H]⁺.

### Synthesis of Compound 176

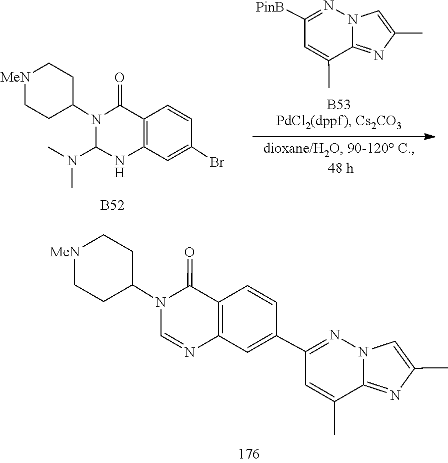

A mixture of 7-bromo-2-(dimethylamino)-3-(1-methylpiperidin-4-yl)-2,3-dihydroquinazolin-4(1H)-one (B52; 90 mg, 0.25 mmol), 2,8-dimethyl-6-(4,4,5,5-tetramethyl-1,3,2-dioxaborolan-2-yl)imidazo[1,2-b]pyridazine (B53; 180 mg, 0.27 mmol), PdCl₂(dppf) (21 mg, 0.026 mmol) and cesium carbonate (240 mg, 0.74 mmol) in dioxane (3.5 mL) and H₂O (0.3 mL) was heated to 90° C. for 16 h, and then heated to 120° C. for 32 hours. Next, the reaction mixture was filtered over a pad of Celite using dimethylformamide as eluent, concentrated in vacuo, and dissolved in 2N aqueous hydrochloric acid (20 mL). The aqueous phase was extracted with dichloromethane (3×15 mL) and filtered under vacuum. The resulting solution was concentrated under reduced pressure and the residue was purified by flash chromatography on a C18 column (12 g) eluting with acetonitrile (5-70%) in 0.1% aqueous formic acid. Fractions containing the product were combined, neutralized with ammonium carbonate, and lyophilized. The resulting solid was triturated with methyl tert-butyl ether (3 mL), followed by ethyl acetate (3 mL), and traces of solvent were removed under reduced pressure, to afford 7-(2,8-dimethylimidazo[1,2-b]pyridazin-6-yl)-3-(1-methylpiperidin-4-yl)quinazolin-4(3H)-one (Compound 176; 31 mg) as a solid. LCMS (ES, m/z): 389.2 [M+H]⁺. ¹H NMR (CH₃OH-d₄, 400 MHz): δ_{H} 8.42 (1H, s), 8.34 (1H, d, J=8.5 Hz), 8.29 (1H, s), 8.20 (1H, d, J=8.5 Hz), 7.95 (1H, s), 7.67 (1H, s), 4.80-4.70 (1H, m) 3.12 (2H, d, J=11.4 Hz), 2.69 (3H, s), 2.50 (3H, s), 2.41 (3H, s), 2.35 (2H, t, J=11.8 Hz), 2.20-2.26 (2H, m), 2.01 (2H, d, J=12.1 Hz).

### Example 21

### Synthesis of Compound 177

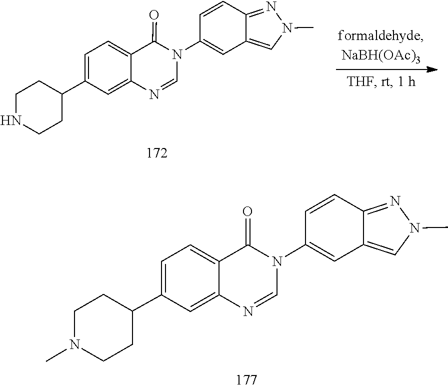

A mixture of 3-(2-methyl-2H-indazol-5-yl)-7-(piperidin-4-yl)quinazolin-4(3H)-one (Compound 172 from Example 25; 185 mg, 0.47 mmol), and formaldehyde (37% in water, 0.19 mL, 2.34 mmol) in dichloromethane (6 mL) and ethanol (2 mL) was stirred at room temperature for 1 h. Sodium triacetoxyborohydride (594 mg, 2.8 mmol) was then added and the mixture was stirred for an additional 1 h at room temperature. The mixture was then diluted with dichloromethane (50 mL), and washed with saturated sodium bicarbonate (2×50 mL) and brine (50 mL). The organic layer was separated, dried over magnesium sulfate, filtered and concentrated under reduced pressure. The crude product was purified by reverse phase chromatography eluting with acetonitrile (5 to 50%) in 0.1% aqueous formic acid, and the resulting solid was dissolved in water (3 mL), neutralized with ammonium carbonate (50 mg, 0.48 mmol) and lyophilized, to afford 3-(2-methyl-2H-indazol-5-yl)-7-(1-methylpiperidin-4-yl)quinazolin-4(3H)-one (Compound 177; 31 mg) as a solid. LCMS (ES, m/z): 374.2 [M+H]⁺. ¹H NMR (DMSO-d₆, 400 MHz): δ_{H} 8.47 (1H, s), 8.35 (1H, s), 8.12 (1H, d, J=8.2 Hz), 7.85 (1H, s), 7.70 (1H, d, J=9.1 Hz), 7.57 (1H, s), 7.50 (1H, d, J=8.3 Hz), 7.28 (1H, d, J=9.2 Hz), 4.21 (3H, s), 2.90 (2H, d, J=11.0 Hz), 2.64-2.70 (1H, m), 2.21 (3H, s), 2.02 (2H, t, J=11.3 Hz), 1.69-1.83 (4H, m).

### Example 22

### Synthesis of Compound 178

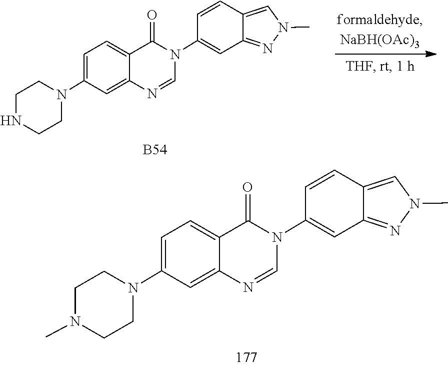

3-(2-Methyl-2H-indazol-6-yl)-7-(piperazin-1-yl)quinazolin-4(3H)-one (B54) was prepared according to the procedure described for the preparation of Compound 153 (see Examples 3 and 4), substituting 2-methyl-2H-indazol-5-amine (B10) with 2-methyl-2H-indazol-6-amine as the starting material. Intermediate B54 was obtained as a solid. LCMS (ES, m/z): 398.1 [M+H]⁺. A mixture of 3-(2-methyl-2H-indazol-6-yl)-7-(piperazin-1-yl)quinazolin-4(3H)-one (B54; 52 mg, 0.13 mmol), and formaldehyde (37% in water, 20 mg, 0.053 mL, 0.66 mmol) in dichloromethane (6 mL) and ethanol (2 mL) was stirred at room temperature for 1 h. Sodium triacetoxyborohydride (167 mg, 0.79 mmol) was then added and the mixture was stirred for 1 h at room temperature. The mixture was then diluted with dichloromethane (50 mL), and washed with saturated sodium bicarbonate (2×50 mL) and brine (50 mL). The organic layer was separated, dried over magnesium sulfate, filtered and concentrated under reduced pressure. The crude product was purified by reverse phase chromatography eluting with acetonitrile (5 to 50%) in 0.1% aqueous formic acid, and the resulting solid was dissolved in water (3 mL), neutralized with ammonium carbonate (50 mg, 0.48 mmol) and lyophilized, to afford 3-(2-methyl-2H-indazol-6-yl)-7-(4-methylpiperazin-1-yl)quinazolin-4(3H)-one (Compound 178; 38 mg) as a solid. LCMS (ES, m/z): 375.2 [M+H]⁺. ¹H NMR (DMSO-d₆, 400 MHz): δ_{H} 8.44 (1H, s), 8.25 (1H, s), 7.97 (1H, d, J=9.0 Hz), 7.80 (1H, d, J=8.8 Hz), 7.72 (1H, s), 7.24 (1H, d, J=9.1 Hz), 7.07 (1H, d, J=8.8 Hz), 7.00 (1H, s), 4.20 (3H, s), 3.39 (4H, m), 2.22 (3H, s). (part of the 4H multiplet can be observed under the DMSO peak. *Or 2.45 (4H, m)).

### Example 23

### Synthesis of Compound 179

### Synthesis of Intermediate B55

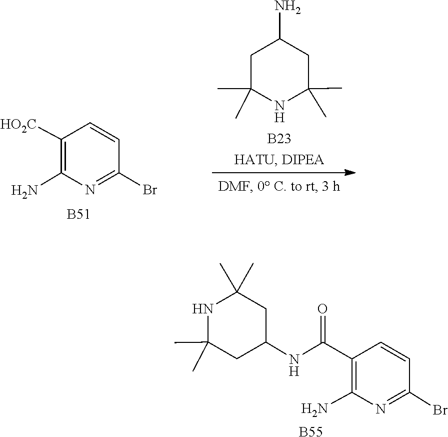

A mixture of 2-amino-6-bromonicotinic acid (B51; 200 mg, 0.92 mmol) and 4-amino-2,2,6,6-tetramethylpiperidine (B23; 160 mg, 1.02 mmol) in dimethylformamide (4.6 mL) was cooled to 0° C., and treated with diisopropylethylamine (500 μL, 2.86 mmol) dropwise, followed by hexafluorophosphate azabenzotriazole tetramethyl uronium (388 mg, 1.02 mmol). The mixture was then warmed to room temperature and stirred for 3 h. The reaction mixture was then diluted with ethyl acetate (20 mL) and washed with saturated aqueous sodium bicarbonate (20 mL) and brine (20 mL). The organic layer was dried over anhydrous sodium sulfate and concentrated in vacuo, to afford 2-amino-6-bromo-N-(2,2,6,6-tetramethylpiperidin-4-yl)nicotinamide (B55; 318 mg) as a solid. LCMS (ES, m/z): 355.1 [M+H]⁺.

### Synthesis of Intermediate B56

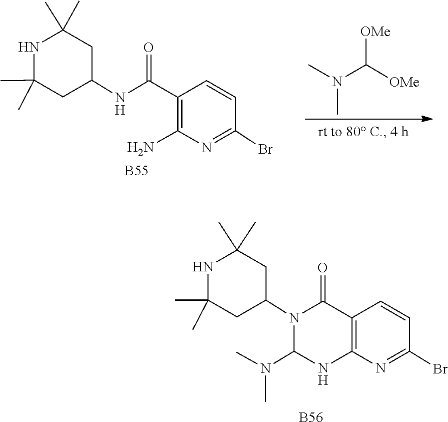

A mixture of 2-amino-6-bromo-N-(1-methylpiperidin-4-yl)nicotinamide (B55; 200 mg, 0.56 mmol) and N,N-dimethylformamide dimethyl acetal (2.5 mL, 18.8 mmol) in a 10 mL sealed tube was heated at 80° C. for 4 h. The reaction mixture was then dissolved in dichloromethane (20 mL) and washed with 20% aqueous sodium hydroxide (15 mL), then brine (20 mL). The organic phase was dried over sodium sulfate and the residue concentrated in vacuo, to afford 7-bromo-2-(dimethylamino)-3-(2,2,6, 6-tetramethylpiperidin-4-yl)-2,3-dihydropyrido[2,3-d]pyrimidin-4(1H)-one (B56; 142 mg) as a solid. LCMS (ES, m/z): 410.1 [M+H]⁺.

### Synthesis of Compound 179

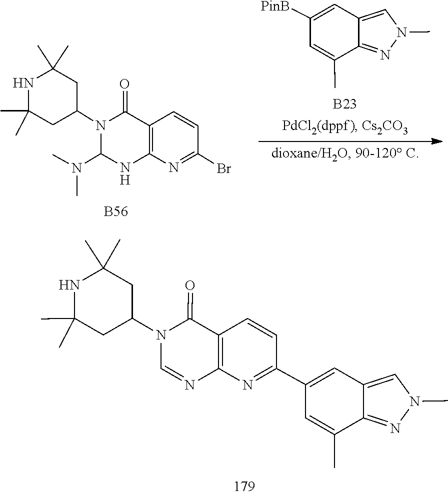

A mixture of 7-bromo-2-(dimethylamino)-3-(2,2,6,6-tetramethylpiperidin-4-yl)-2,3-dihydropyrido[2,3-d]pyrimidin-4(1H)-one (B56; 130 mg, 0.32 mmol), 2,7-dimethyl-5-(4,4,5,5-tetramethyl-1,3,2-dioxaborolan-2-yl)-2H-indazole (B23; 95 mg, 0.25 mmol), PdCl₂(dppf) (26 mg, 0.03 mmol) and cesium carbonate (310 mg, 0.95 mmol) in dioxane (4.5 mL) and H₂O (0.4 mL) was heated to 90° C. for 16 h under a nitrogen atmosphere, and then heated to 120° C. overnight. Next, the reaction mixture was diluted with ethyl acetate (25 mL) and washed with saturated sodium bicarbonate (15 mL) and brine (15 mL), and the organic phase was dried over sodium sulfate and concentrated in vacuo. The residue was purified by flash chromatography on a silica gel column (12 g) eluting with methanol (5-50%) in dichloromethane. The fractions containing the product were combined and the solvent was removed under reduced pressure, and the residue was triturated with methyl tert-butyl ether, and traces of solvent were removed in vacuo, to afford 7-(2,7-dimethyl-2H-indazol-5-yl)-3-(2,2,6,6-tetramethylpiperidin yl)pyrido[2,3-d]pyrimidin-4(3H)-one (Compound 179; 75 mg) as a solid. LCMS (ES, m/z): 431.3 [M+H]⁺. ¹H NMR (CHCl₃-d, 300 MHz): δ 8.65 (1H, d, J=8.4 Hz), 8.44 (1H, s), 8.37 (1H, s), 8.01 (4H, m), 5.40 (1H, m), 4.29 (3H, s), 2.73 (3H, s), 1.99 (2H, d, J=11.8 Hz), 1.47 (6H, s), 1.36 (8H, m).

### Example 24

### Synthesis of Compound 180

### Synthesis of Intermediate B57

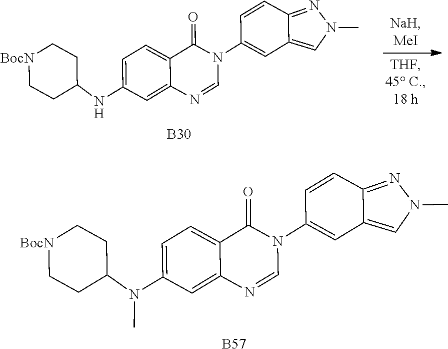

A mixture of tert-butyl 4-((3-(2-methyl-2H-indazol-5-yl)-4-oxo-3,4-dihydroquinazolin-7-yl)amino)piperidine-1-carboxylate (B30 from Example 13; 83 mg, 0.18 mmol) and sodium hydride 60% (11 mg, 0.26 mmol) in tetrahydrofuran (3 mL) was stirred for 1 h at room temperature in a sealed tube. Iodomethane (13 μL, 0.21 mmol) was then added and the mixture was heated to 45° C. overnight. Next, ethyl acetate (50 mL) and saturated sodium bicarbonate (50 mL) were added, and the organic layer was separated, washed with saturated sodium bicarbonate (50 mL) and brine (50 mL), dried over magnesium sulfate, filtered and concentrated under reduced pressure. The resulting solid was purified by preparative HPLC eluting with acetonitrile (10 to 100%) in 0.1% aqueous formic acid, to afford tert-butyl 4-(methyl(3-(2-methyl-2H-indazol-5-yl)-4-oxo-3,4-dihydroquinazolin-7-yl)amino)piperidine-1-carboxylate (B57; 13 mg) as a solid. LCMS (ES, m/z): 489.2 [M+H]⁺.

### Synthesis of Compound 180

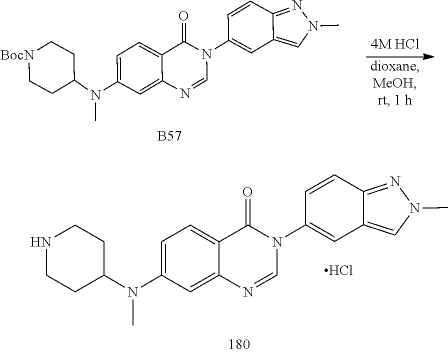 [SMILES: "B57" ; CN(c1ccc2c(=O)n(-c3ccc4nn(C)cc4c3)cnc2c1)C1CCN(C(=O)OC(C)(C)C)CC1 ; CN(c1ccc2c(=O)n(-c3ccc4nn(C)cc4c3)cnc2c1)C1CCNCC1 ; Cl]

tert-Butyl 4-(methyl(3-(2-methyl-2H-indazol-5-yl)-4-oxo-3,4-dihydroquinazolin-7-yl)amino)piperidine-1-carboxylate (B57; 13 mg, 0.03 mmol) was added to a mixture of 4M hydrochloric acid in dioxane (2 mL) and methanol (1 mL), and the reaction mixture was stirred at room temperature for 1 h. The volatiles were then removed under reduced pressure and the crude product was suspended in ether (1 mL). The mixture was centrifuged, decanted and the resulting hydrochloride salt was added to water (1 mL) and lyophilized to afford 7-(methyl(piperidin-4-yl)amino)-3-(2-methyl-2H-indazol-5-yl)quinazolin-4(3H)-one, hydrochloride (Compound 180 HCl salt); 7 mg) as a solid. LCMS (ES, m/z): 389.1 [M+H]⁺. ¹H NMR (CH₃OH-d₄, 400 MHz): δ_{H} 9.56 (1H, s), 8.60 (1H, s), 8.16 (1H, d, J=9.2 Hz), 8.08 (1H, s), 7.82 (1H, d, J=9.2 Hz), 7.56 (1H, d, J=9.2 Hz), 7.42 (1H, d, J=9.4 Hz), 7.02 (1H, d, J=2.4 Hz), 4.43 (1H, t, J=11.3 Hz), 4.34 (3H, s), 3.63-3.74 (1H, m), 3.56 (2H, d, J=12.9 Hz), 3.35 (2H, d, J=12.9 Hz), 3.06 (3H, s), 2.19 (2H, q, J=12.9 Hz), 2.04 (2H, d, J=13.6 Hz). (Signals for hydrogen atoms of HCl salt are exchanging with the residual water from the CH₃OH-d₄).

### Example 25

### Synthesis of Compound 181

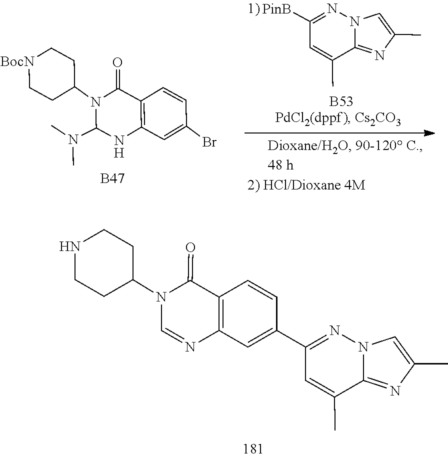

A mixture of tert-butyl 4-(7-bromo-2-(dimethylamino)-4-oxo-1,4-dihydroquinazolin-3(2H)-yl)piperidine-1-carboxylate (B47 from Example 27, 90 mg, 0.2 mmol), 2,8-dimethyl-6-(4,4,5,5-tetramethyl-1,3,2-dioxaborolan-2-yl)imidazo[1,2-b]pyridazine (B53; 150 mg, 0.23 mmol), PdCl₂(dppf) (18 mg, 0.02 mmol) and cesium carbonate (200 mg, 0.61 mmol) in dioxane (2.8 mL) and H₂O (0.2 mL) was heated to 90° C. for 16 h, and then heated to 120° C. for 32 hours. The reaction mixture was then concentrated under reduced pressure and the residue was purified by flash chromatography on a silica gel column (12 g) eluting with methanol (2-25%) in dichloromethane. Fractions containing the product were combined and evaporated under reduced pressure, and the resulting solid was stirred vigorously in a 4M solution of hydrochloric acid in dioxane (2 mL) at room temperature for 6 h. The volatiles were then removed in vacuo and the residue was treated with water (15 mL) and dichloromethane (15 mL). The aqueous phase was washed with dichloromethane (2×15 mL), concentrated in vacuo, and the resulting HCl salt was purified by reverse phase flash chromatography on a C18 column (12 g) eluting with acetonitrile (5-70%) in 0.1% aqueous formic acid. Fractions containing the product were combined, neutralized with ammonium carbonate, and lyophilized. The resulting solid was triturated with methyl tert-butyl ether (3 mL), then ethyl acetate (3 mL), and traces of solvent were removed under reduced pressure, to afford 7-(2,8-dimethylimidazo[1,2-b]pyridazin-6-yl)-3-(piperidin yl)quinazolin-4(3H)-one (Compound 181; 25 mg, 0.065 mmol) as a solid. LCMS (ES, m/z): 375.2 [M+H]⁺. ¹H NMR (CH₃OH-d₄, 400 MHz): δ 8.53 (1H, s), 8.41-8.30 (3H, m), 8.23 (1H, d, J=8.5 Hz), 7.97 (1H, s), 7.70 (1H, s), 3.51 (2H, d, J=13.1 Hz), 3.12 (2H, t, J=12.8 Hz), 2.69 (3H, s), 2.50 (3H, s), 2.40 (2H, d, J=13.5 Hz), 2.16 (2H, d, J=13.1 Hz). (*Methine proton of piperidine substituent hidden under water peak).

### Example 26

### Synthesis of Compound 185

### Synthesis of Intermediate B2

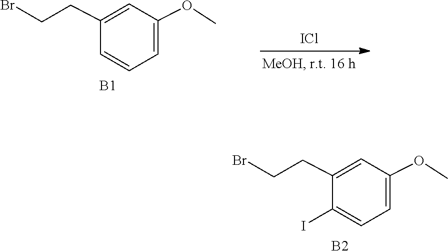

A solution of 1-(2-bromoethyl)-3-methoxybenzene (B1; 3.9 g, 0.018 mmol) and iodine monochloride (2.94 g, 0.018 mmol) in methanol (50 mL) was stirred for 16 h at 25° C., and then concentrated under reduced pressure. The resulting mixture was extracted with dichloromethane (50 mL) and washed with aqueous sodium sulfate (50 mL), and then extracted with H₂O (3×60 mL), and concentrated under vacuum. The residue was purified by reverse flash chromatography on a silica gel column eluting with methanol (10% to 50% gradient over 10 min) in water, to afford 2-(2-bromoethyl)-1-iodo-4-methoxybenzene (B2; 4 g) as an oil. LCMS: (ES, m/z): 341[M+1]⁺.

### Synthesis of Intermediate B4

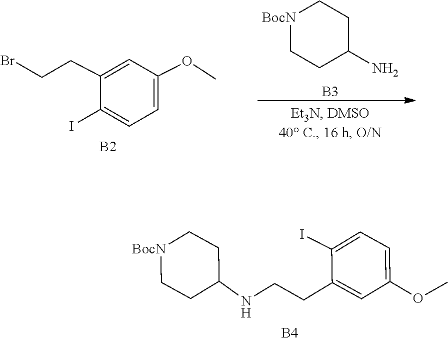

A solution of 2-(2-bromoethyl)-1-iodo-4-methoxybenzene (B2; 3.8 g, 11 mmol), tert-butyl 4-aminopiperidine-1-carboxylate (B3; 2.68g), and triethylamine (3.38 g, 33 mmol) in dimethylsulfoxide (50 mL) was stirred for 16 h at 40° C., and then cooled to 25° C. The resulting mixture was extracted with ethyl acetate (3×30 mL), and the combined organic layers were washed with H₂O (3×30 mL) and concentrated under vacuum. The residue was purified by reverse flash chromatography on a silica gel column, eluting with methanol (10% to 50% gradient over 10 min) in water, to afford 1-benzyl-4-[2-(5-ethyl-2-methylphenyl) ethyl]piperidine (B4; 1.9 g) as an oil. LCMS: (ES, m/z): 461[M+1]⁺.

### Synthesis of Intermediate B5

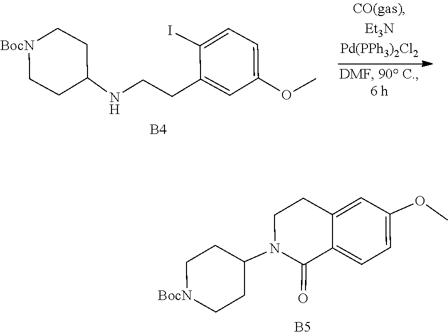

A solution of tert-butyl 4-[[2-(2-iodo-5-methoxyphenyl) ethyl]amino]piperidine-1-carboxylate (B4; 1.4 g, 3 mmol), triethylamine (0.92 g) and Pd(PPh₃)₂Cl₂ (0.43 g, 0.001 mmol) in dimethylformamide (20 mL) was stirred for 6 h at 90° C. under a carbon monoxide atmosphere. The mixture was then cooled to 25° C. and extracted with ethyl acetate (20 mL) and H₂O (3×20 mL), then concentrated under vacuum. The residue was purified by reverse flash chromatography on a silica gel column, eluting with methanol (10% to 50% gradient over 10 min) to afford tert-butyl 4-(7-methoxy-1-oxo-3,4-dihydroisoquinolin-2-yl) piperidine-1-carboxylate (B5; 740 mg) as an oil. LCMS: (ES, m/z): 361[M+1]⁺.

### Synthesis of Intermediate B6

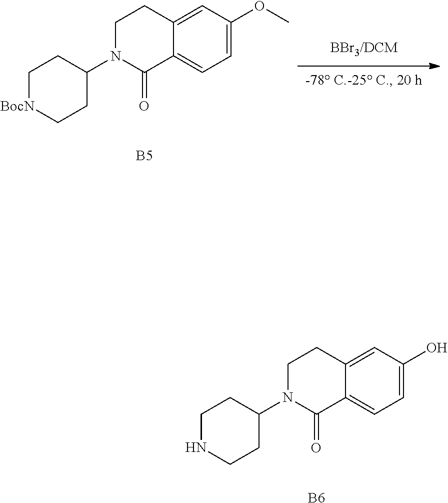

A mixture of tert-butyl 4-(6-methoxy-1-oxo-3,4-dihydro-2H-naphthalen-2-yl)piperidine-1-carboxylate (B5; 900 mg, 2.5 mmol) in dichloromethane (10 mL) was treated with boron tribromide (1.88 g, 7.5 mmol) at −78° C., and the mixture was then stirred for 20 h at 25° C. The reaction was quenched with methanol at 0° C., and neutralized using sodium hydroxide (2.5N). The resulting mixture was concentrated under reduced pressure, to afford 6-hydroxy-2-(piperidin-4-yl) -3,4-dihydro-2H-naphthalen-1-one (B6; 8.5 g) as a solid. LCMS: (ES, m/z): 247[M+1]⁺.

### Synthesis of Intermediate B7

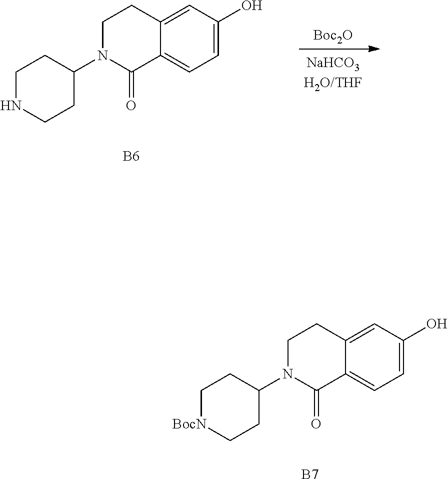

A mixture of 6-hydroxy-2-(piperidin-4-yl)-3,4-dihydroisoquinolin-1-one (B6; 600 mg, 2.4 mmol) and sodium bicarbonate (614 mg, 7.3 mmol) in tetrahydrofuran (10 mL) was stirred for 30 min at 25° C. Next, a mixture of di-tert-butyl dicarbonate (1.06 g, 4.9 mmol) in water (10 mL) was added in portions at 25° C. Volatiles were then removed under reduced pressure, and the aqueous layer was extracted with ethyl acetate (3×10 mL), and the resulting mixture was concentrated under vacuum to afford tert-butyl 4-(1,6-dihydroxy-octahydro-1H-isoquinolin yl)piperidine-1-carboxylate (B7; 120 mg) as a solid. LCMS: (ES, m/z): 347[M+1]⁺.

### Synthesis of Intermediate B8

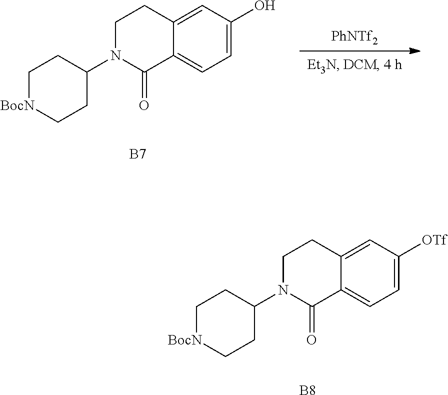

A mixture of tert-butyl 4-(6-hydroxy-1-oxo-3,4-dihydroisoquinolin-2-yl) piperidine-1-carboxylate (B7; 130 mg, 0.38 mmol) and triethylamine (114 mg, 1.13 mmol) in dichloromethane (5 mL) was treated with 1,1,1-trifluoro-N-phenyl-N-trifluoromethane sulfonyl methane sulfonamide (147 mg, 0.41 mmol) in portions, and then stirred for 4 h at 25° C. The aqueous layer was then extracted with ethyl acetate and H₂O (3×15 mL), and the resulting mixture was concentrated under vacuum, to afford tert-butyl 4-[1-oxo-6-(trifluoromethanesulfonyloxy)-3,4-dihydroisoquinolin-2-yl]piperidine-1-carboxylate (B8; 150 mg) as a solid. LCMS: (ES, m/z): 479[M+1]⁺.

### Synthesis of Intermediate B10

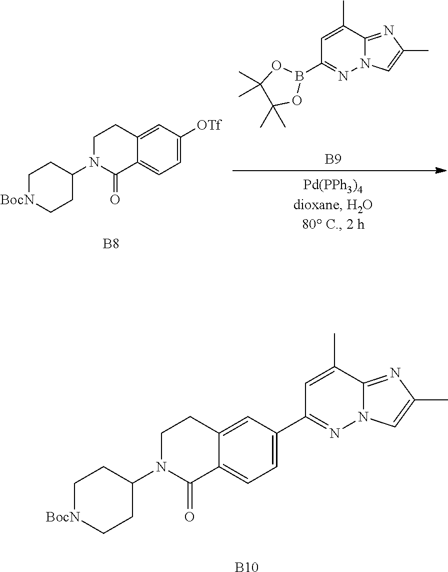

A mixture of tert-butyl 4-[1-oxo-6-(trifluoromethanesulfonyloxy)-3,4-dihydroisoquinolin-2-yl]piperidine-1-carboxylate (B8; 140 mg, 0.29 mmol), 2,8-dimethyl-6-(4,4,5,5-tetramethyl -1,3,2-dioxaborolan-2-yl) imidazo[1,2-b]pyridazine (B9; 96 mg, 0.35 mmol), Pd(PPh₃)₄ (101 mg, 0.09 mmol), and potassium carbonate (121 mg, 0.88 mmol) in dioxane (10 mL) and H₂O (2 mL) was stirred for 2 h at 80° C. The mixture was then cooled to 25° C., and the aqueous layer was extracted with ethyl acetate and H₂O (3×20 mL). The resulting mixture was concentrated under vacuum, and purified by reverse flash chromatography on a silica gel column eluting with methanol (10% to 50% gradient over 10 min) in water; to afford tert-butyl 4-(6-[2,8-dimethylimidazo[1,2-b]pyridazin-6-yl]-1-oxo-3,4-dihydroisoquinolin-2-yl) piperidine-1-carboxylate (B10; 60 mg) as a solid. LCMS (ES, m/z): 476[M+1]⁺.

### Synthesis of Compound 185

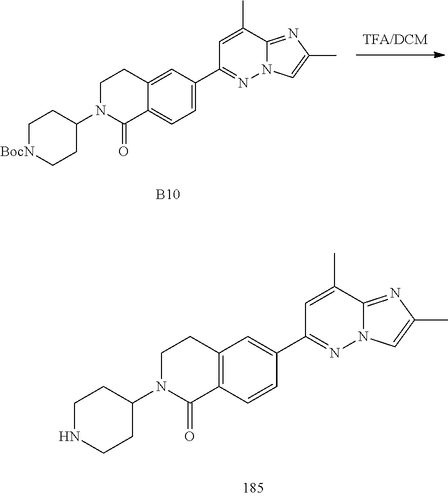

A mixture of tert-butyl4-(6[2,8-dimethylimidazo[1,2-b]pyridazin-6-yl ]-1-oxo-3,4-dihydroisoquinolin-2-yl)piperidine-1-carboxylate (B10; 90 mg, 0.2 mmol) in dichloromethane (6 mL) was treated with trifluoroacetic acid (65 mg, 0.6 mmol) in portions over 3 h at 25° C. The resulting mixture was concentrated under reduced pressure, and purified by reverse flash chromatography on a C18 column eluting with methanol (10% to 50% gradient over 10 min) in water, to afford 6-[2,8-dimethylimidazo [1,2-b] pyridazin-6-yl ]-2-(piperidin-4-yl) -3,4-dihydroisoquinolin-1-one (Compound 100; 4.4 mg) as a solid. LCMS: (ES, m/z): 376[M+1]⁺. ¹H NMR (400 MHz, DMSO-d₆, ppm) δ 8.08 (d, J=1.0 Hz, 1H), 8.03-7.96 (m, 3H), 7.69 (d, J=1.2 Hz, 1H), 3.50 (t, J=6.5 Hz, 2H), 3.04 (t, J=6.4 Hz, 5H), 2.64-2.54 (m, 5H), 2.42 (d, J=0.9 Hz, 3H), 1.69-1.59 (m, 2H), 1.54 (d, J=11.4 Hz, 2H).

### Example 27

### Synthesis of Compound 186

### Synthesis of Intermediate B58

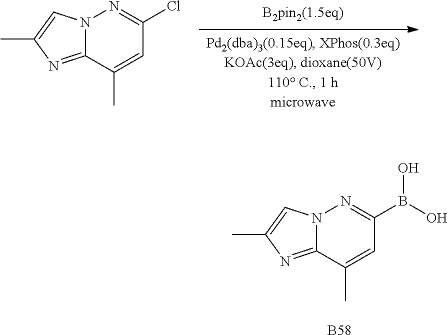

6-chloro-2,8-dimethylimidazo[1,2-b]pyridazine (400 mg, 2.20 mmol), B₂PIN₂ (839.1 mg, 3.30 mmol), KOAc (648.4 mg, 6.60 mmol), X-Phos (314.9 mg, 0.66 mmol), Pd₂(dba)₃ (341.9 mg, 0.33 mmol), and dioxane (15 mL) were combined in a sealed tube under a nitrogen atmosphere. The reaction mixture was irradiated with microwave radiation for 1 h at 110° C. The resulting mixture was filtered, and the filtrate concentrated in vacuo to afford product. LCMS (ES, m/z): 192 [M+H]⁺.

### Synthesis of Intermediate B59

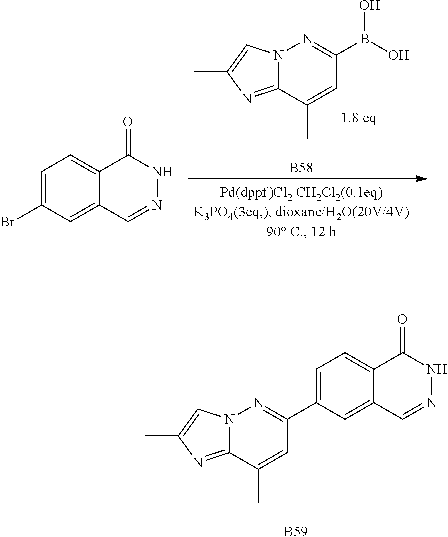 [SMILES: O=c1[nH]ncc2cc(Br)ccc12 ; "B59" ; "B58" ; Cc1cn2nc(-c3ccc4c(=O)[nH]ncc4c3)cc(C)c2n1 ; Cc1cn2nc(B(O)O)cc(C)c2n1]

A mixture of 6-bromo-2H-phthalazin-1-one (350.0 mg, 1.55 mmol), 2,8-dimethylimidazo[1,2-b]pyridazin-6-ylboronic acid (534.7 mg, 2.80 mmol), K₃PO₄ (990.4 mg, 4.66 mmol), and Pd(dppf)Cl₂ CH₂Cl₂ (126.70 mg, 0.15 mmol) in dioxane (15.00 mL) and H₂O (3.00 mL) was stirred for 12 h at 90° C. under a N2 atmosphere, then diluted with H₂O (50 mL) and extracted with DCM (3×50 mL). The combined organic layers were washed with saturated NaCl (1×50 mL), dried over anhydrous Na₂SO₄, and filtered. The filtrate was concentrated in vacuo to give a residue. The residue was purified by silica gel column chromatography, eluted with DCM/MeOH (92/8) to afford 6-[2,8-dimethylimidazo[1,2-b]pyridazin-6-yl]-2H-phthalazin-1-one (330 mg, 58.2%) as a solid. LCMS (ES, m/z): 292 [M+H]⁺.

### Synthesis of Intermediate B60

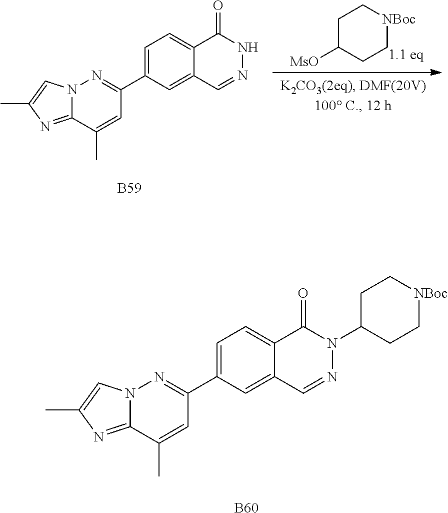 [SMILES: "B59" ; Cc1cn2nc(-c3ccc4c(=O)[nH]ncc4c3)cc(C)c2n1 ; Cc1cn2nc(-c3ccc4c(=O)n(C5CCN(C(=O)OC(C)(C)C)CC5)ncc4c3)cc(C)c2n1 ; "B60" ; CC(C)(C)OC(=O)N1CCC(OS(C)(=O)=O)CC1]

A mixture of 6-[2,8-dimethylimidazo[1,2-b]pyridazin-6-yl]-2H-phthalazin-1-one (300 mg, 1.03 mmol), tert-butyl 4-(methanesulfonyloxy)piperidine-1-carboxylate (316.4 mg, 1.13 mmol) and K₂CO₃ (284.6 mg, 2.06 mmol) in DMF (6 mL) was stirred for 12 h at 100° C., then diluted with H₂O (20 mL) and extracted with ethyl acetate (3×20 mL). The combined organic layers were washed with brine (30 mL), dried over anhydrous Na₂SO₄, and filtered. The filtrate was concentrated in vacuo to give a residue. The residue was purified by silica gel column chromatography, eluted with PE/EtOAc (9/1) to afford tert-butyl 4-(6-[2,8-dimethylimidazo[1,2-b]pyridazin-6-yl]-1-oxophthalazin-2-yl)piperidine-1-carboxylate (40 mg, 8.1%) as a solid. LCMS (ES, m/z): 475 [M+H]⁺.

### Synthesis of Compound 186

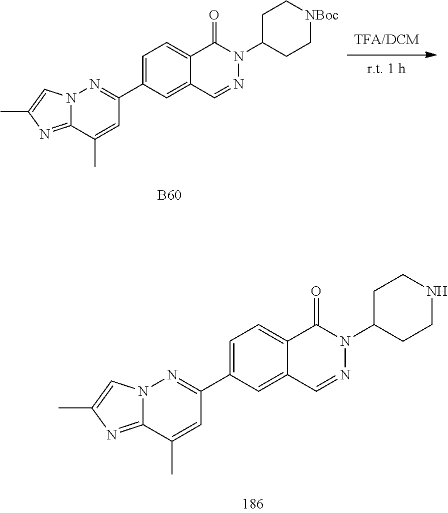 [SMILES: Cc1cn2nc(-c3ccc4c(=O)n(C5CCN(C(=O)OC(C)(C)C)CC5)ncc4c3)cc(C)c2n1 ; "B60" ; Cc1cn2nc(-c3ccc4c(=O)n(C5CCNCC5)ncc4c3)cc(C)c2n1]

A mixture of tert-butyl 4-(6-[2,8-dimethylimidazo[1,2-b]pyridazin-6-yl]-1-oxophthalazin-2-yl)piperidine-1-carboxylate (36.0 mg, 0.07 mmol) and TFA (0.40 mL) in DCM (1.60 mL) was stirred for 1 h at room temperature. The resulting mixture was concentrated in vacuo to give a residue. The residue was purified by Prep-HPLC (Condition 1, Gradient 1) to afford 6-[2,8-dimethylimidazo[1,2-b]pyridazin-6-yl]-2-(piperidin-4-yl)phthalazin-1-one (4.8 mg, 16.6%) as a white solid. LCMS (ES, m/z): 375 [M+H]⁺. ¹H NMR (400 MHz, DMSO-d₆) δ 8.65-8.58 (m, 2H), 8.51 (dd, J=8.4, 1.8 Hz, 1H), 8.41 (d, J=8.5 Hz, 1H), 8.14 (d, J=1.0 Hz, 1H), 7.81 (d, J=1.2 Hz, 1H), 5.03-4.92 (m, 1H), 3.08 (d, J=12.3 Hz, 2H), 2.69-2.58 (m, 5H), 2.44 (d, J=0.8 Hz, 3H), 2.09 (s, 1H), 1.87 (qd, J=12.1, 4.1 Hz, 2H), 1.71 (d, J=11.3 Hz, 2H).

### Example 28

### Synthesis of Compound 191

### Synthesis of Intermediate B61

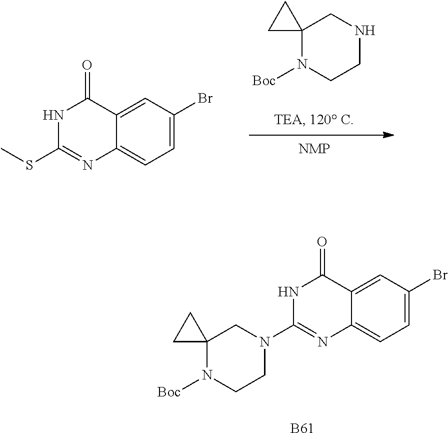 [SMILES: CSc1nc2ccc(Br)cc2c(=O)[nH]1 ; CC(C)(C)OC(=O)N1CCNCC12CC2 ; "B61" ; CC(C)(C)OC(=O)N1CCN(c2nc3ccc(Br)cc3c(=O)[nH]2)CC12CC2]

To a solution of 6-bromo-2-(methylthio)quinazolin-4(3H)-one (200 mg, 0.74 mmol) in NMP (1 mL) was added tert-butyl 4,7-diazaspiro[2.5]octane-4-carboxylate (188 mg, 0.89 mmol) followed by Et₃N (0.206 mL, 1.48 mmol). The reaction mixture was heated at 120° C. for 5 days, then cooled in ice, and water (4 mL) was added dropwise. The resulting suspension was stirred for 1 h, the solid collected by filtration, rinsed with water, and dried. The collected material was purified by silica gel column chromatography using a gradient of 0 to 50% of ethyl acetate in hexanes to provide tert-butyl 7-(6-bromo-4-oxo-3,4-dihydroquinazolin-2-yl)-4,7-diazaspiro[2.5]octane-4-carboxylate (181 mg, 56%). LCMS (ES, m/z): 434.9, 436.9 [M+H]⁺. ¹H NMR (DMSO-d₆, 400 MHz): δ_{H} 11.46 (1H, s), 7.95 (1H, s), 7.70 (1H, d, J=8.8 Hz), 7.21 (1H, d, J=8.5 Hz), 3.62 (2H, s), 3.50 (2H, s), 3.47 (2H, s), 1.41 (9H, s), 0.90 (2H, s), 0.84 (2H, s).

### Synthesis of Intermediate B62

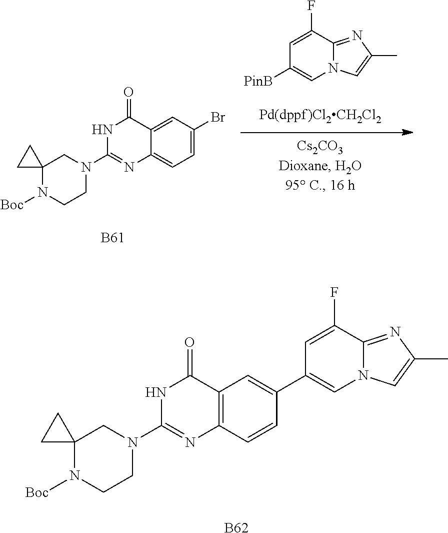

Argon was bubbled into a mixture of tert-butyl 7-(6-bromo-4-oxo-3,4-dihydroquinazolin-2-yl)-4,7-diazaspiro[2.5]octane-4-carboxylate (86 mg, 0.20 mmol), 8-fluoro-2-methyl-6-(4,4,5,5-tetramethyl-1,3,2-dioxaborolan-2-yl)imidazo[1,2-a]pyridine (74 mg, 0.27 mmol) and dioxane (2.3 mL). Water (0.1 mL) was added, followed by Cs₂CO₃ (161 mg, 0.49 mmol) and Pd(dppf)Cl2CH₂Cl₂ (16 mg, 0.020 mmol). The reaction mixture was purged with argon for 10 min and heated at 95° C. for 16 h, then allowed to cool to room temperature. DMF was added to the cooled reaction mixture, followed by dropwise addition of 1 N HCl to pH 7. The reaction mixture was filtered through Celite, rinsed with DMF and the filtrate was concentrated in vacuo to give a residue. The residue was purified by silica gel column chromatography using a gradient of 0 to 20% of MeOH in CH₂Cl₂ to provide tert-butyl 7-(6-(8-fluoro-2-methylimidazo[1,2-a ]pyridin-6-yl)-4-oxo-3,4-dihydroquinazolin-2-yl)-4,7-diazaspiro[2.5]octane-4-carboxylate (53 mg, 53%). LCMS (ES, m/z): 505.3 [M+H]³⁰ . ¹ H NMR (DMSO-d₆, 400 MHz): δ_{H} 11.43 (1H, s), 8.84 (1H, s), 8.20 (1H, s), 7.95 (1H, d, J=8.6 Hz), 7.80 (1H, s), 7.54 (1H, d, J=12.6 Hz), 7.37 (1H, s), 3.65 (2H, s), 3.51 (4H, s), 2.36 (3H, s), 1.42 (9H, s), 0.92 (2H, s), 0.87 (2H, s).

### Synthesis of Compound 191

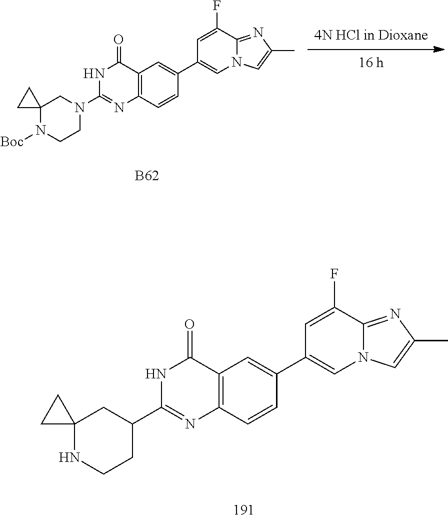 [SMILES: "B62" ; Cc1cn2cc(-c3ccc4nc(N5CCN(C(=O)OC(C)(C)C)C6(CC6)C5)[nH]c(=O)c4c3)cc(F)c2n1 ; Cc1cn2cc(-c3ccc4nc(C5CCNC6(CC6)C5)[nH]c(=O)c4c3)cc(F)c2n1 ; "191"]

To tert-butyl 7-(6-(8-fluoro-2-methylimidazo[1,2-a]pyridin-6-yl)-4-oxo-3,4-dihydroquinazolin-2-yl)-4,7-diazaspiro[2.5]octane-4-carboxylate (38 mg, 0.075 mmol) was added 4 N HCl in dioxane (2 mL). The reaction mixture was stirred for 2 h, then concentrated in vacuo to give a residue. The residue was taken up in water, filtered through a 45 μM syringe filter, and the pH adjusted to 6-7 with 2 N Na₂CO₃. A precipitate formed and was collected by filtration, rinsed with water, and dried to afford 6-(8-fluoro-2-methylimidazo[1,2-a]pyridin-6-yl)-2-(4,7-diazaspiro[2.5]octan-7-yl)quinazolin-4(3H)-one (18 mg, 67%). LCMS (ES, m/z): 405.1 [M+H]⁺. ¹H NMR (DMSO-d₆, 400 MHz): δ_{H} 8.83 (1H, s), 8.19 (1H, s), 7.92 (1H, d, J=8.6 Hz), 7.80 (1H, s), 7.53 (1H, d, J=12.6 Hz), 7.33 (1H, d, J=8.3 Hz), 3.61 (2H, s), 3.49 (2H, s), 2.81 (2H, s), 2.36 (3H, s), 0.54 (2H, s), 0.46 (2H, s).

### Example 29

### Synthesis of Compound 190

### Synthesis of Intermediate

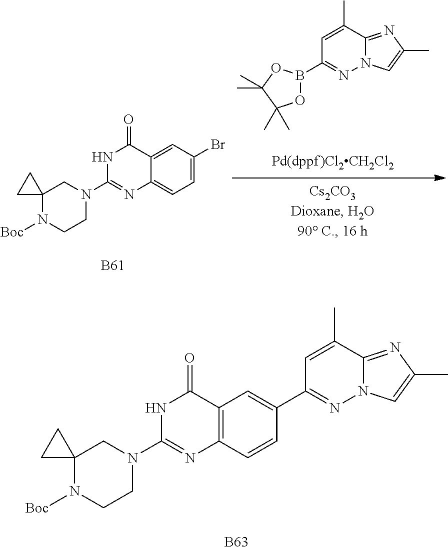

Argon was bubbled into a mixture of tert-butyl 7-(6-bromo-4-oxo-3,4-dihydroquinazolin-2-yl)-4,7-diazaspiro[2.5]octane-4-carboxylate (90 mg, 0.20 mmol), 2,8-dimethyl-6-(4,4,5,5-tetramethyl-1,3,2-dioxaborolan-2-yl)imidazo[1,2-b]pyridazine (79 mg, 0.29 mmol) and dioxane (2.2 mL). Water (0.1 mL) was added, followed by Cs₂CO₃ (168 mg, 0.52 mmol) and Pd(dppf)Cl₂.CH₂Cl₂ (16.9 mg, 0.021 mmol). The reaction mixture was purged with argon for 10 min and heated at 95° C. for 16 h, then allowed to cool to room temperature. DMF was added to the cooled reaction, followed by dropwise addition of 1 N HCl to pH 6-7. The reaction mixture was filtered through Celite, rinsed with DMF and the filtrate concentrated in vacuo to give a residue. The residue was purified by silica gel column chromatography using a gradient of 10 to 30% of MeOH in CH₂Cl₂ to afford tert-butyl 7-(6-(2,8-dimethylimidazo[1,2-b]pyridazin-6-yl)-4-oxo-3,4-dihydroquinazolin-2-yl)-4,7-diazaspiro[2.5]octane-4-carboxylate (43 mg, 41%). LCMS (ES, m/z): 502.0 [M+H]⁺. ¹H NMR (CHCl₃-d, 400 MHz): δ_{H} 10.57 (1H, s), 8.58 (1H, s), 8.31 (1H, dd, J=8.7, 2.2 Hz), 7.76 (1H, s), 7.50 (1H, d, J=8.7 Hz), 7.36 (1H, s), 3.76 (4H, d, J=13.5 Hz), 3.64 (2H, s), 2.72 (3H, s), 2.53 (3H, s), 1.50 (9H, s), 1.12 (4H, d, J=7.7 Hz).

### Synthesis of Compound 190

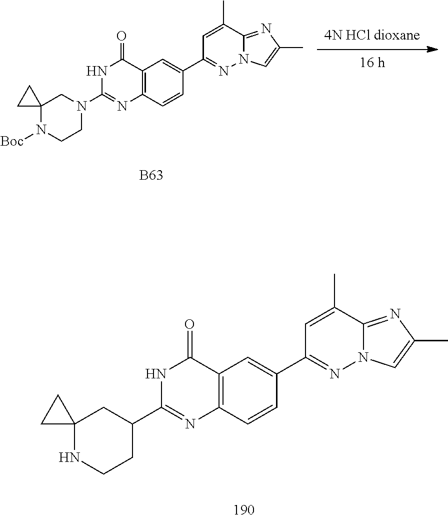 [SMILES: "B63" ; Cc1cn2nc(-c3ccc4nc(N5CCN(C(=O)OC(C)(C)C)C6(CC6)C5)[nH]c(=O)c4c3)cc(C)c2n1 ; Cc1cn2nc(-c3ccc4nc(C5CCNC6(CC6)C5)[nH]c(=O)c4c3)cc(C)c2n1 ; "190"]

To tert-butyl 7-(6-(2,8-dimethylimidazo[1,2-b]pyridazin-6-yl)-4-oxo-3,4-dihydroquinazolin-2-yl)-4,7-diazaspiro[2.5]octane-4-carboxylate (43 mg, 0.086 mmol) was added 4 N HCl in dioxane (2 mL). The reaction mixture was stirred for 16 h, then concentrated in vacuo, taken up in water, and the pH adjusted to 6-7 with 1 N NaOH. A precipitate formed and was collected by filtration, rinsed with water, and dried. The solid was suspended in ethyl acetate (4 mL), stirred for 2 h then collected by filtration, rinsed with ethyl acetate and dried to afford 642,8-dimethylimidazo[1,2-b]pyridazin-6-yl)-2-(4,7-diazaspiro[2.5]octan-7-yl)quinazolin-4(3H)-one (19 mg, 55%). LCMS (ES, m/z): 402.1 [M+H]⁺. ¹H NMR (DMSO-d₆, 400 MHz): δ_{H} 8.54 (1H, s), 8.24 (1H, dd, J=8.7, 2.3 Hz), 8.02 (1H, s), 7.69 (1H, s), 7.35 (1H, d, J=8.6 Hz), 3.63 (2H, s), 3.51 (2H, s), 2.81 (2H, s), 2.58 (3H, s), 2.38 (3H, s), 0.54 (2H, s), 0.45 (2H, s).

### Example 30

### Synthesis of Compound 204

### Synthesis of Intermediate B75

tert-butyl 4-((3-(2-methyl-2H-indazol-6-yl)-4-oxo-3,4-dihydroquinazolin-7-yl)amino)piperidine-1-carboxylate was prepared using the procedure described for 152 (Example 3). 2-methyl-2H-indazol-5-amine was substituted for 2-methyl-2H-indazol-6-amine in the first step of Example 3, and the procedure described for the preparation of 152 (i.e. steps 2 and 3) were subsequently applied, with 1-methylpiperazine was substituted for tert-butyl 4-aminopiperidine-1-carboxylate in step 3. tert-butyl 4-((3-(2-methyl-2H-indazol-6-yl)-4-oxo-3,4-dihydroquinazolin-7-yl)amino)piperidine-1-carboxylate was obtained as a solid. LCMS (ES, m/z): 475.0 [M+H]⁺.

### Synthesis of Compound 204

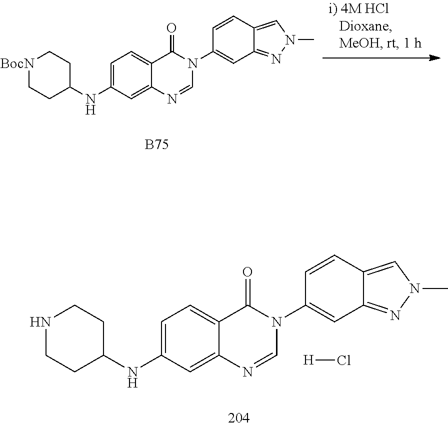

To a solution of tert-butyl 4-((3-(2-methyl-2H-indazol-6-yl)-4-oxo-3,4-dihydroquinazolin-7-yl)amino)piperidine-1-carboxylate (109 mg, 0.23 mmol) in methanol (2.0 mL) was added 4 M HCl in dioxane (3 mL). The reaction mixture was stirred at room temperature for 1 h, then concentrated in vacuo to give a residue. The residue was purified by reverse phase chromatography using a gradient from 5 to 50% of acetonitrile in water containing 0.1% hydrochloric acid to afford 3-(2-methyl-2H-indazol-6-yl)-7-(piperidin-4-ylamino)quinazolin-4(3H)-one (22 mg, 26%) as a solid. LCMS (ES, m/z): 375.1 [M+H]⁺. ¹H NMR (CH₃OH-d₄, 400 MHz): δ_{H} 8.58 (1H, s), 8.34 (1H, s), 8.04 (1H, d, J=8.9 Hz), 7.88 (1H, d, J=8.8 Hz), 7.74 (1H, s), 7.14 (1H, dd, J=8.8, 1.8 Hz), 6.97-7.00 (1H, m), 6.79 (1H, d, J=2.2 Hz), 4.27 (3H, s), 3.84 (1H, m), 3.49 (2H, d, J=12.8 Hz), 3.23 (2H, t, J=12.6 Hz), 2.31 (2H, d, J=14.2 Hz), 1.77 (2H, q, J=11.9 Hz).

### Example 31

### Synthesis of Compound 203

### Synthesis of Compound 203

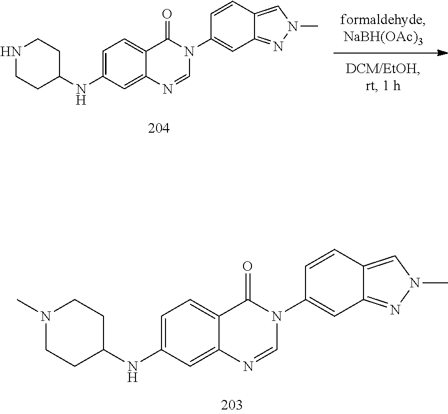

A mixture of 3-(2-methyl-2H-indazol-6-yl)-7-(piperidin-4-ylamino)quinazolin-4(3H)-one (45 mg, 0.12 mmol), and formaldehyde (37% in water, 20 mg, 0.049 mL, 0.60 mmol) in DCM (6 mL) and ethanol (2 mL) was stirred at room temperature for 1 h. NaBH(OAc)₃ (153 mg, 0.72 mmol) was added, and the reaction mixture was stirred at room temperature for and additional 1 h, diluted with DCM (50 mL), and washed with saturated NaHCO₃ (2×50 mL) and brine (50 mL). The organic layer was separated, dried over MgSO₄, filtered, and concentrated in vacuo to give a residue. The residue was purified by normal phase chromatography using a gradient of 10 to 50% (EtOAc/10% MeOH) and DCM with 1% Et3N additive to afford 3-(2-methyl-2H-indazol-6-yl)-7-((1-methylpiperidin-4-yl)amino)quinazolin-4(3H)-one (17 mg, 36%) as a solid. LCMS (ES, m/z): 389.2 [M+H]⁺. ¹H NMR (DMSO-d₆, 400 MHz): δ_{H} 8.43 (1H, s), 8.17 (1H, s), 7.81 (2H, dd, J=21.5, 8.8 Hz), 7.69 (1H, s), 7.05 (1H, d, J=8.8 Hz), 6.83 (1H, d, J=8.9 Hz), 6.61-6.63 (2H, m), 4.20 (3H, s), 2.75 (2H, d, J=10.9 Hz), 2.18 (3H, s), 2.06 (2H, t, J=11.3 Hz), 1.91 (2H, d, J=12.4 Hz), 1.40-1.49 (2H, m).

### Example 32

### Synthesis of Compound 192

### Synthesis of Intermediate B81

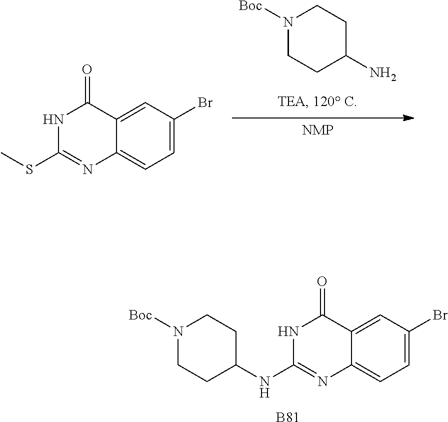

To a solution of 6-bromo-2-(methylthio)quinazolin-4(3H)-one (200 mg, 0.74 mmol), in NMP (1 mL) was added tert-butyl 4-aminopiperidine-1-carboxylate (354 mg, 1.78 mmol) followed by Et₃N (0.2 mL, 1.48 mmol). The reaction mixture was heated at 120° C. for 7 days, then allowed to cool to room temperature. Water was added to the reaction mixture, and a precipitate formed that was collected by filtration, rinsed with water, and dried. The solid was purified by silica gel column chromatography using a gradient from 0 to 10% of MeOH in CH₂Cl₂ to afford tert-butyl 4-((6-bromo-4-oxo-3,4-dihydroquinazolin-2-yl)amino)piperidine-1-carboxylate (193 mg, 62%). LCMS (ES, m/z): 423.1, 425.1 [M+H]⁺. ¹H NMR (DMSO-d₆, 400 MHz): δ_{H} 10.82 (1H, s), 7.92 (1H, s), 7.66 (1H, d,=8.8 Hz), 7.19 (1H, d,=8.7 Hz), 6.38 (1H, s), 3.96 (1H, m), 3.82 (2H, d, J=12.8 Hz), 2.94 (2H, br s), 1.89 (2H, d, J=11.9 Hz), 1.40 (9H, s), 1.31-1.37 (2H, m).

### Synthesis of Intermediate B82

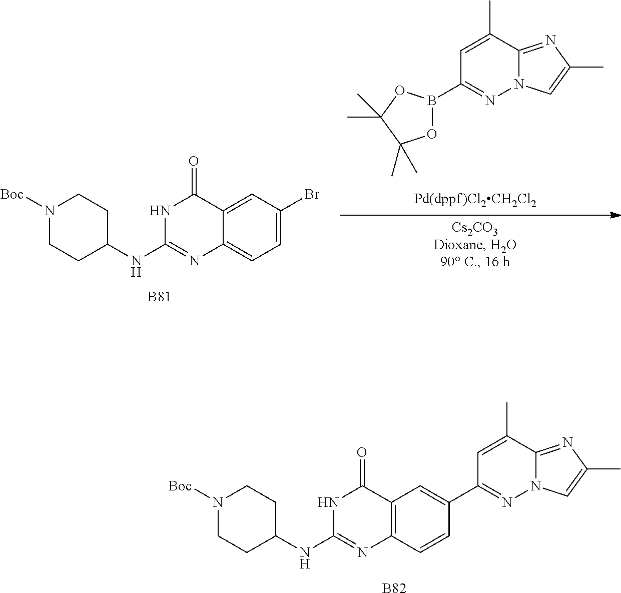

Argon was bubbled into a mixture of tert-butyl 4-((6-bromo-4-oxo-3,4-dihydroquinazolin-2-yl)amino)piperidine-1-carboxylate (99 mg, 0.23 mmol), 2,8-dimethyl-6-(4,4,5,5-tetramethyl-1,3,2-dioxaborolan-2-yl)imidazo[1,2-b]pyridazine (89.4 mg, 0.33 mmol) and dioxane (2.3 mL). Water (0.12 mL) was added, followed by Cs₂CO₃ (191 mg, 0.59 mmol) and Pd(dppf)Cl₂.CH₂Cl₂ (19 mg, 0.023 mmol). The reaction mixture was purged with argon for 10 min and heated at 95° C. for 16 h. DMF was added to the cooled reaction followed by dropwise addition of 1 N HCl to pH 7. The reaction mixture was filtered through Celite, rinsed with DMF and the filtrate was concentrated in vacuo to a residue. The residue was purified by silica gel column chromatography using a gradient from 0 to 20% of MeOH in CH₂Cl₂ to afford tert-butyl 4-((6-(2,8-dimethylimidazo[1,2-b]pyridazin-6-yl)-4-oxo-3,4-dihydroquinazolin-2-yl)amino)piperidine-1-carboxylate (28 mg, 25%). LCMS (ES, m/z): 490.3 [M+H]⁺. ¹H NMR (DMSO-d₆, 400 MHz): δ_{H} 10.79 (1H, s), 8.53 (1H, d, J=2.2 Hz), 8.24 (1H, dd, J=8.7, 2.2 Hz), 8.04 (1H, s), 7.67 (1H, s), 7.37 (1H, d, J=8.7 Hz), 6.49 (1H, s), 4.02 (1H, m), 3.85 (2H, d, J=13.0 Hz), 2.96 (3H, br s), 2.59 (3H, s), 2.39 (2H, s), 1.93 (2H, d, J=12.3 Hz), 1.40 (9H, s), 1.31-1.37 (2H, m).

### Synthesis of Compound 192

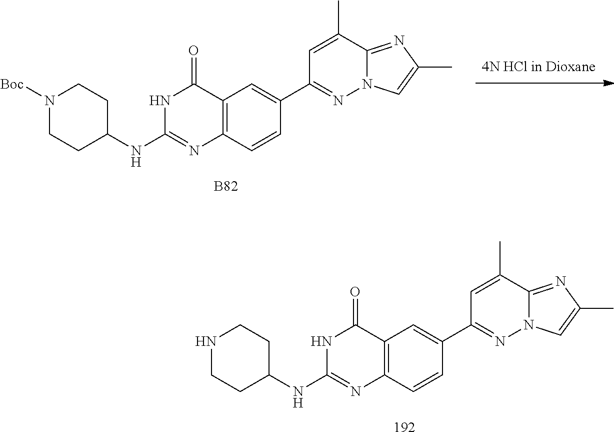

To tert-butyl 4-(2-(2,7-dimethyl-2H-indazol-5-yl)-4-oxo-3,4-dihydroquinazolin-7-yl)piperazine-1-carboxylate (28 mg, 0.57 mmol) was added 4 N HCl in dioxane (2 mL). The reaction mixture was stirred for 2 h and concentrated in vacuo to a residue. The residue was taken up in water (3 mL), filtered through a 45 μM syringe filter, and pH adjusted to approximately 7 with 2 N Na₂CO₃. A precipitate formed, collected by filtration, and dried to afford 6-(2,8-dimethylimidazo[1,2-b]pyridazin-6-yl)-2-(piperidin-4-ylamino)quinazolin-4(3H)-one (13 mg, 61%). LCMS (ES, m/z): 390.2 [M+H]⁺. ¹H NMR (DMSO-d₆, 400 MHz): δ_{H} 8.51 (1H, d, J=2.1 Hz), 8.21 (1H, dd, J=8.7, 2.2 Hz), 8.03 (1H, s), 7.66 (1H, s), 7.35 (1H, d, J=8.7 Hz), 6.63 (1H, br s), 3.89 (1H, m), 2.93 (2H, dt, J=12.3, 3.6 Hz), 2.58 (3H, s), 2.55 (2H, t, J=11.3 Hz), 2.38 (3H, s), 1.88 (2H, d, J=11.6 Hz) 1.28-1.36 (2H, m).

### Example 33

### Synthesis of Compound 193

### Synthesis of Intermediate B83

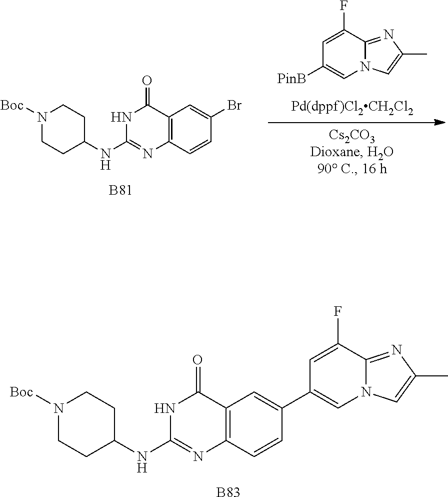

Argon was bubbled into a mixture of tert-butyl 4-((6-bromo-4-oxo-3,4-dihydroquinazolin-2-yl)amino)piperidine-1-carboxylate (90 mg, 0.21 mmol), 8-fluoro-2-methyl-6-(4,4,5,5-tetramethyl-1,3,2-dioxaborolan-2-yl)imidazo[1,2-a]pyridine (57.9 mg, 0.21 mmol) and dioxane (2.1 mL). Water (0.1 mL) was added, followed by Cs₂CO₃ (174 mg, 0.53 mmol) and Pd(dppf)Cl₂.CH₂Cl₂ (17.4 mg, 0.021 mmol). The reaction mixture was purged with argon for 10 min and heated at 95° C. for 16, then allowed to cool to room temperature. DMF was added to the reaction mixture followed by dropwise addition of 1 N HCl to pH 7. The reaction mixture was filtered through Celite, rinsed with DMF, and the filtrate was concentrated in vacuo to a residue. The residue was purified by silica gel column chromatography using a gradient from 80 to 100% of ethyl acetate in hexane to afford tert-butyl 4-((6-(8-fluoro-2-methylimidazo[1,2-a]pyridin-6-yl)-4-oxo-3,4-dihydroquinazolin-2-yl)amino)piperidine-1-carboxylate (62 mg, 59%). LCMS (ES, m/z): 493.0 [M+H]⁺. ¹H NMR (DMSO-d₆, 400 MHz): δ_{H} 10.75 (1H, s), 8.81 (1H, s), 8.16 (1H, d, J=2.3 Hz), 7.91 (1H, dd, J=8.5, 2.3 Hz), 7.81 (1H, d, J=2.9 Hz), 7.51 (1H, d, J=12.6 Hz), 7.35 (1H, d, J=8.6 Hz), 6.39 (1H, s), 4.00 (1H, br s), 3.84 (2H, d, J=13.3 Hz), 2.96 (2H, br s), 2.36 (3H, s), 1.93 (2H, d, J=12.3 Hz), 1.40 (9H, s), 1.31-1.36 (2H, m).

### Synthesis of Compound 193

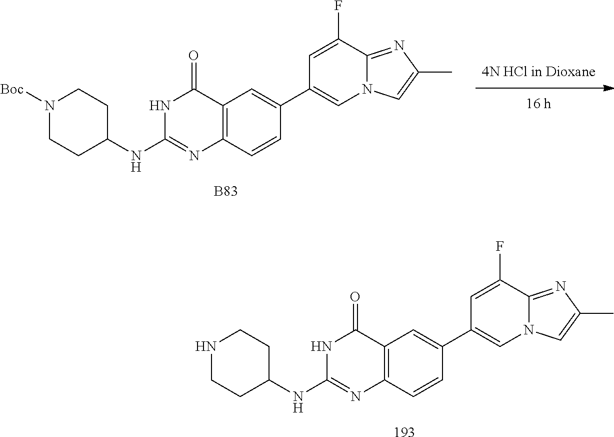

To tert-butyl 4-((6-(8-fluoro-2-methylimidazo[1,2-a]pyridin-6-yl)-4-oxo-3,4-dihydroquinazolin-2-yl)amino)piperidine-1-carboxylate (62 mg, 0.13 mmol) was added 4 N HCl in dioxane (2 mL). The reaction mixture was stirred for 12 h, then concentrated in vacuo to a residue, the residue dissolved in water, and filtered through a 40 μm syringe filter. The filtered solution was neutralized to pH 6-7 with 1 N NaOH. A precipitate formed that was collected by filtration, rinsed with water, and allowed to dry. The solid was purified by a silica gel column chromatography using a gradient from 0 to 30% of MeOH in 2% Et₃N in CH₂Cl₂ to afford 6-(8-fluoro-2-methylimidazo[1,2-a]pyridin-6-yl)-2-(piperidin-4-ylamino)quinazolin-4(3H)-one (22 mg, 45%). LCMS (ES, m/z): 393.1 [M+H]⁺. ¹H NMR (DMSO-d₆, 400 MHz): δ_{H} 8.81 (1H, s), 8.17 (1H, s), 7.92 (1H, d, J=8.6 Hz), 7.80 (1H, s), 7.51 (1H, d, J=12.6 Hz), 7.33 (1H, d, J=8.6 Hz), 6.84 (1H, s), 4.07 (1H, br s), 3.23-3.25 (2H, m), 3.00 (2H, t, J=11.7 Hz), 2.35 (3H, s), 2.08 (2H, br s), 1.62 (2H, br s).

### Example 34

### Synthesis of Compound 205

### Synthesis of Compound 205

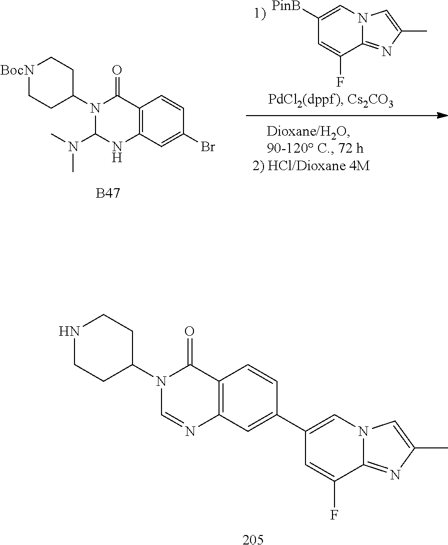

Tert-butyl 4-(7-bromo-2-(dimethylamino)-4-oxo-1,4-dihydroquinazolin-3(2H)-yl)piperidine-1-carboxylate (130 mg, 0.29 mmol), 8-fluoro-2-methyl-6-(4,4,5,5-tetramethyl-1,3,2-dioxaborolan-2-yl)imidazo[1,2-a]pyridine (95 mg, 0.34 mmol), PdCl₂(dppf) (21 mg, 0.029 mmol) and Cs₂CO₃ (467 mg, 1.43 mmol) were combined in dioxane (3.3 mL) and H₂O (340 μL) in a sealed tube and heated at 90° C. for 16 h, then at 120° C. for 48 hours. The reaction mixture was allowed to cool to room temperature and concentrated in vacuo to give a residue. The residue was purified by flash chromatography on a silica gel column using a gradient of 0-20% MeOH in DCM. Selected fractions were combined and concentrated in vacuo. To the resulting solid was added HCl 4 M in dioxane (2 mL), and the solution was stirred vigorously at room temperature for 6 hours, then concentrated in vacuo to give a residue. To the residue was added water (15 mL) and DCM (15 mL). The aqueous phase was washed with DCM (2×15 mL) and neutralized with (NH₄)₂CO₃ to form a suspension. The resulting suspension was cooled to 4° C., the precipitate collected by vacuum filtration, washed with cold water, and dried under high vacuum at room temperature overnight to afford 7-(8-fluoro-2-methylimidazo[1,2-a]pyridin-6-yl)-3-(piperidin yl)quinazolin-4(3H)-one (40 mg, 37%) as a solid. LCMS (ES, m/z): 378.2 [M+H]⁺. ¹H NMR (DMSO-d₆, 300 MHz): δ 9.02 (1H, s), 8.49 (1H, s), 8.23 (1H, d, J=8.5 Hz), 8.04 (1H, s), 7.92 (1H, d, J=9.0 Hz), 7.86 (1H, s), 7.70 (1H, d, J=12.7 Hz), 4.70 (1H, m), 3.09 (2H, d, J=12.7 Hz), 2.39 (3H, s), 1.79-1.96 (4H, m).

### Example 35

### Synthesis of Compound 182

### Synthesis of Intermediate B90

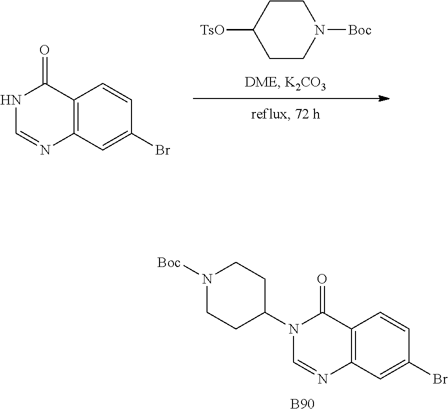

7-bromoquinazolin-4(3H)-one (2.53 g, 11.2 mmol), tert-butyl 4-(tosyloxy)piperidine-1-carboxylate (12.0 g, 33.8 mmol), and K₂CO₃ (4.67 g, 33.8 mmol) were dissolved in DME (150 mL) and refluxed for 72 hours. The reaction mixture was filtered through a pad of Celite and the filter cake washed with ethyl acetate (100 mL). The filtrate was concentrated in vacuo to give a residue and the residue was purified by flash chromatography on an silica gel column using a gradient of 0-70% EtOAc in hexane to afford tert-butyl 4-(7-bromo-4-oxoquinazolin-3(4H)-yl)piperidine-1-carboxylate (2.16 g, 47%) as a solid. LCMS (ES, m/z): 408.1, 410.1 [M+H]⁺.

### Synthesis of Compound 182

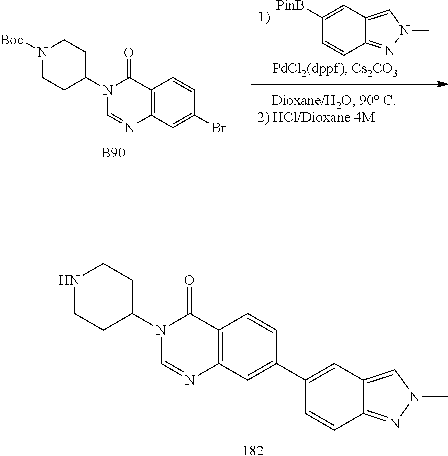

Tert-butyl 4-(7-bromo-4-oxoquinazolin-3(4H)-yl)piperidine-1-carboxylate (100 mg, 0.25 mmol), 2-methyl-5-(4,4,5,5-tetramethyl-1,3,2-dioxaborolan-2-yl)-2H-indazole (76 mg, 0.29 mmol), PdCl₂(dppf) (18 mg, 0.024 mmol), and Cs₂CO₃ (239 mg, 0.74 mmol) was dissolved in dioxane (2.8 mL) and H₂O (280 μL) and heated at 90° C. for 4 h under argon atmosphere. The reaction mixture was diluted with ethyl acetate (25 mL) and washed with saturated NaHCO₃ (20 mL) and brine (2×20 mL). The organic phase was then filtered, dried over Na₂SO₄, and concentrated in vacuo to give a residue. The residue was purified by flash chromatography on a silica gel column using a gradient of 0-20% MeOH in DCM. Selected fractions were combined and evaporated in vacuo to afford a solid. To solid was added HCl 4 M in dioxane (5 mL), and the resulting mixture was stirred vigorously for 2 hours, then concentrated in vacuo and redissolved in water (10 mL). The aqueous layer was extracted with DCM (2×10 mL) and neutralized with (NH₄)₂CO₃to form a suspension. The resulting suspension was cooled to 4° C. for 4 hours, and the precipitate collected by vacuum filtration, washed with cold water, and dried under high vacuum overnight to afford 7-(2-methyl-2H-indazol-5-yl)-3-(piperidin-4-yl)quinazolin-4(3H)-one (57 mg, 65%) as a solid. LCMS (ES, m/z): 360.2 [M+H]⁺. ¹H NMR (DMSO-d₆, 300 MHz): δ 8.46 (2H, m), 8.18-8.23 (2H, m), 7.94 (2H, m), 7.72 (2H, m), 4.71 (1H, m), 4.21 (3H, s), 3.10 (2H, m), 2.61 (2H, m), 1.92 (2H, m), 1.79 (2H, m).

### Example 36

### Synthesis of Compound 206

### Synthesis of Compound 206

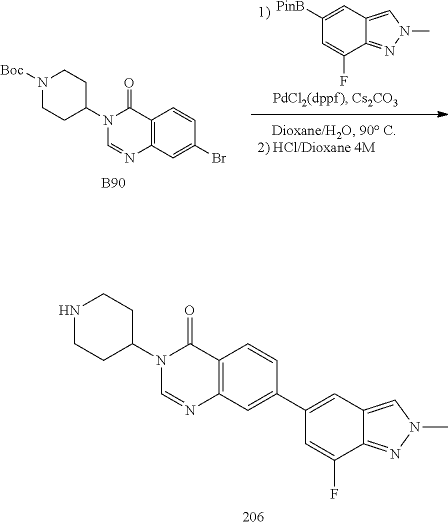

Tert-butyl 4-(7-bromo-4-oxoquinazolin-3(4H)-yl)piperidine-1-carboxylate (100 mg, 0.25 mmol), 7-fluoro-2-methyl-5-(4,4,5,5-tetramethyl-1,3,2-dioxaborolan-2-yl)-2H-indazole (81 mg, 0.29 mmol), PdCl₂(dppf) (18 mg, 0.024 mmol), and Cs₂CO₃ (239 mg, 0.74 mmol) were dissolved in dioxane (2.8 mL) and H₂O (280 μL) and heated at 90° C. for 4 h under argon atmosphere in a sealed tube. The reaction mixture was diluted with ethyl acetate (25 mL) and washed with saturated NaHCO₃ (20 mL) and brine (2×20 mL). The organic phase was filtered, dried over Na₂SO₄, and concentrated in vacuo to give a residue. The residue was purified by flash chromatography on a silica gel column using a gradient of 0-20% MeOH in DCM. Selected fractions were combined and concentrated in vacuo to yield a solid. To the solid was added HCl 4 M in dioxane (5 mL) and the resulting mixture was stirred vigorously for 2 h. The reaction mixture was concentrated in vacuo to give a residue and the residue was dissolved with water (10 mL). This aqueous solution was extracted with DCM (2×10 mL) and neutralized with (NH₄)₂CO₃ to form a suspension. The resulting suspension was cooled to 4° C. for 4 hours and the resulting precipitate collected by vacuum filtration, washed with cold water, and dried under high vacuum overnight to afford 7-(7-fluoro-2-methyl-2H-indazol-5-yl)-3-(piperidin yl)quinazolin-4(3H)-one (75 mg, 81%) as a solid. LCMS (ES, m/z): 378.2 [M+H]⁺. ¹H NMR (DMSO-d₆, 300 MHz): δ 8.59 (1H, d, J=3.0 Hz), 8.47 (1H, s), 8.21 (1H, d, J=8.4 Hz), 8.06 (1H, s), 7.99 (1H, s), 7.93 (1H, d, J=8.4 Hz), 7.57 (1H, d, J=13.0 Hz), 4.70 (1H, m), 4.24 (3H, s), 3.10 (2H, m), 2.59 (2H, m), 1.92 (2H, m), 1.79 (2H, m).

### Example 37

### Synthesis of Compound 207

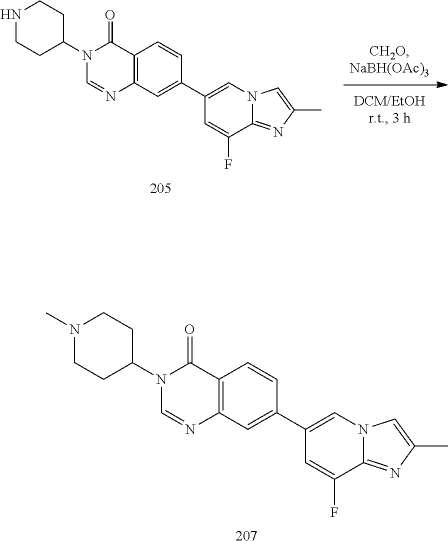

7-(8-fluoro-2-methylimidazo[1,2-a]pyridin-6-yl)-3-(piperidin-4-yl)quinazolin-4(3H)-one (18 mg, 0.048 mmol) was dissolved in a mixture of DCM (500 μL) and EtOH (150 μL). To this solution was added formaldehyde (37% in water, 20 mg, 0.24 mmol). The reaction mixture was stirred at room temperature for 1 h, then NaBH(OAc)3 (61 mg, 0.29 mmol) was added, and the reaction mixture was stirred for an additional 2 h at room temperature. The reaction mixture was diluted with ethyl acetate (20 mL) and washed with saturated NaHCO₃ (2×15 mL) and brine (2×15 mL). The organic layer was dried over Na₂SO₄ and the solvent was removed in vacuo to give a residue. The residue was purified by flash chromatography on a silica gel column using a gradient of 5-30% MeOH in DCM to afford 7-(8-fluoro-2-methylimidazo[1,2-a]pyridin-6-yl)-3-(1-methylpiperidin-4-yl)quinazolin-4(3H)-one (12 mg, 64%) as a solid. LCMS (ES, m/z): 392.2 [M+H]⁺. ¹H NMR (DMSO-d₆, 300 MHz): δ 9.01 (1H, s), 8.52 (1H, s), 8.23 (1H, d, J=8.4 Hz), 8.04 (1H, s), 7.92 (1H, d, J=8.7 Hz), 7.86 (1H, s), 7.70 (1H, d, J=12.6 Hz), 4.60 (1H, m), 2.95 (2H, m), 2.39 (3H, s), 2.24 (3H, s), 2.11 (4H, m), 1.82 (2H, m).

### Example 38

### Synthesis of Compound 208

### Synthesis of Compound 208

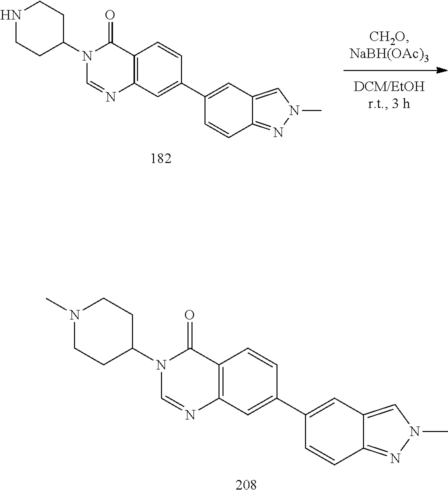

To 7-(2-methyl-2H-indazol-5-yl)-3-(piperidin-4-yl)quinazolin-4(3H)-one (20 mg, 0.056 mmol) in a mixture of DCM (520 μL) and EtOH (170 μL) was added formaldehyde (37% in water, 23 mg, 0.28 mmol). The reaction mixture was stirred at room temperature for 1 h, then NaBH(OAc)₃ (71 mg, 0.33 mmol) was added, and the reaction mixture was stirred for an additional 2 h at room temperature. The reaction mixture was diluted with ethyl acetate (20 mL) and washed with saturated NaHCO₃ (2×15 mL) and brine (2×15 mL). The organic layer was dried over Na₂SO₄, filtered, and concentrated in vacuo to give a residue. The residue was purified by flash chromatography on a silica gel column using a gradient of 5-30% MeOH in DCM to afford 7-(2-methyl-2H-indazol-5-yl)-3-(1-methylpiperidin-4-yl)quinazolin-4(3H)-one (17 mg, 81%) as a solid. LCMS (ES, m/z): 374.2 [M+H]⁺. ¹H NMR (DMSO-d₆, 300 MHz): δ 8.48 (2H, d, J=10.8 Hz), 8.18-8.22 (2H, m), 7.92 (2H, d, J=11.7 Hz), 7.71 (2H, s), 4.61 (1H, m), 4.21 (3H, s), 2.95 (2H, m), 2.25 (3H, s), 2.11 (4H, m), 1.82 (2H, m).

### Example 39

### Synthesis of Compound 209

### Synthesis of Compound 209

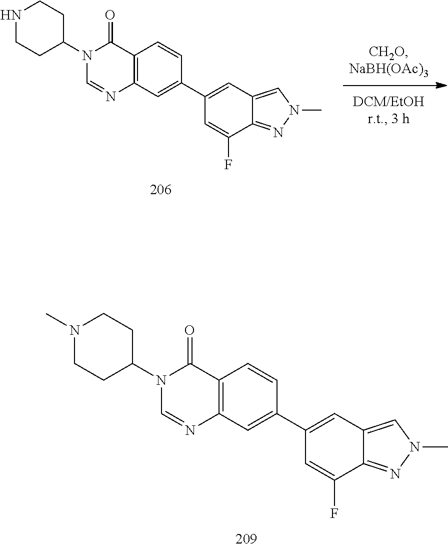

To 7-(7-fluoro-2-methyl-2H-indazol-5-yl)-3-(piperidin-4-yl)quinazolin-4(3H)-one (25 mg, 0.066 mmol) in a mixture of DCM (620 μL) and EtOH (210 μL) was added formaldehyde (37% in water, 27 mg, 0.33 mmol). The reaction mixture was stirred at room temperature for 1 h, then NaBH(OAc)₃ (84 mg, 0.40 mmol) was added, and the reaction mixture was stirred for an additional 2 h at room temperature. The reaction mixture was diluted with ethyl acetate (20 mL) and washed with saturated NaHCO₃ (2×15 mL) and brine (2×15 mL). The organic layer was dried over Na₂SO₄, filtered, and concentrated in vacuo to give a residue. The residue was purified by flash chromatography on a silica gel column using a gradient of 5-30% MeOH in DCM to afford 7-(7-fluoro-2-methyl-2H-indazol-5-yl)-3-(1-methylpiperidin-4-yl)quinazolin-4(3H)-one (21 mg, 80%) as a solid. LCMS (ES, m/z): 392.2 [M+H]⁺. ¹H NMR (DMSO-d₆, 300 MHz): δ 8.59 (1H, s), 8.50 (1H, s), 8.21 (1H, d, J=8.3 Hz), 8.06 (1H, s), 7.98 (1H, s), 7.93 (1H, d, J=8.7 Hz), 7.56 (1H, d, J=13.3 Hz), 4.61 (1H, m), 4.24 (3H, s), 2.95 (2H, m), 2.24 (3H, s), 2.10 (4H, m), 1.82 (2H, m).

### Example 40

### Synthesis of Compound 210

### Synthesis of Intermediate B91

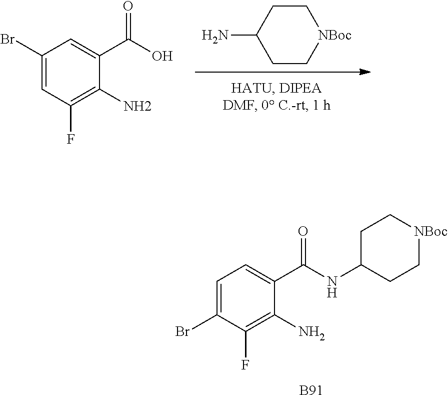 [SMILES: Nc1c(F)cc(Br)cc1C(=O)O ; CC(C)(C)OC(=O)N1CCC(N)CC1 ; "B91" ; CC(C)(C)OC(=O)N1CCC(NC(=O)c2ccc(Br)c(F)c2N)CC1]

To a solution of 2-amino-5-bromo-3-fluorobenzoic acid (1.00 g, 4.27 mmol) and tert-butyl 4-aminopiperidine-1-carboxylate (899 mg, 4.49 mmol) in DMF (20 mL) were added DIPEA (2.23 mL, 12.8 mmol) and HATU (1.95 g, 5.13 mmol) sequentially. The reaction mixture was stirred at 0° C. for 1 h, then partitioned between ethyl acetate (100 mL) and aqueous NH₄Cl saturated (100 ml). The organic layer was separated, washed with aqueous NH₄Cl (sat) (50 ml), aqueous NaHCO₃ (sat) (50 ml), and brine (50 mL), dried over MgSO₄, filtered, and concentrated in vacuo to afford tert-butyl 4-(2-amino-4-bromo-3-fluorobenzamido)piperidine-1-carboxylate (1.76 g, 99%) as a solid. LCMS (ES, m/z): 438.1, 440.1 [M+Na]⁺.

### Synthesis of Intermediate B92

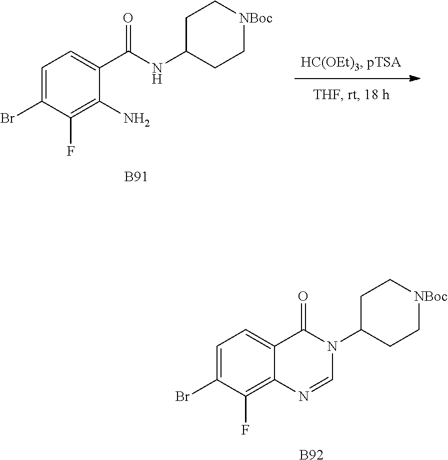 [SMILES: "B91" ; CC(C)(C)OC(=O)N1CCC(NC(=O)c2ccc(Br)c(F)c2N)CC1 ; "B92" ; CC(C)(C)OC(=O)N1CCC(n2cnc3c(F)c(Br)ccc3c2=O)CC1]

To a solution of tert-butyl 4-(2-amino-4-bromo-3-fluorobenzamido)piperidine-1-carboxylate (1.70 g, 4.1 mmol) in THF (40 mL) was added triethylorthofomate (6.05 g, 40.8 mmol) and pTSA (0.08g, 0.41 mmol). The reaction mixture was stirred at room temperature for 18 h, then diluted with ethyl acetate (200 mL), washed with NaHCO₃ (sat) (2×50 ml) and brine (50 mL), dried over MgSO₄, filtered, and concentrated in vacuo to afford tert-butyl 4-(7-bromo-8-fluoro-4-oxoquinazolin-3(4H)-yl)piperidine-1-carboxylate (1.7 g, 98%) as a solid. LCMS (ES, m/z): 426.1, 428.1 [M+H]⁺.

### Synthesis of Intermediate B93

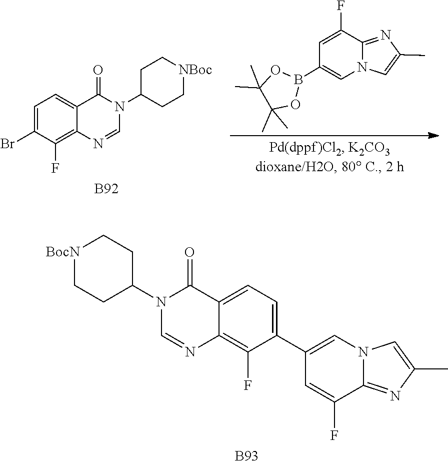

A mixture of tert-butyl 4-(7-bromo-8-fluoro-4-oxoquinazolin-3(4H)-yl)piperidine-1-carboxylate (154 mg, 0.36 mmol), 8-fluoro-2-methyl-6-(4,4,5,5-tetramethyl-1,3,2-dioxaborolan-2-yl)imidazo[1,2-a]pyridine (100 mg, 0.36 mmol), Pd(dppf)Cl₂ (26 mg, 0.036 mmol) and K₂CO₃ (150 mg, 1.08 mmol) in a mixture of dioxane (4.0 mL) and H₂O (1.0 mL) was heated to 80° C. for 2 h and then cooled to room temperature. The reaction mixture was diluted with ethyl acetate (50 mL), and washed with water (25 mL) and brine (25 mL). The organic layer was separated, dried over MgSO₄, filtered, and concentrated in vacuo to give a residue and the residue was purified by normal phase flash chromatography using 0-10% MeOH/DCM gradient to afford tert-butyl 4-(8-fluoro-7-(8-fluoro-2-methylimidazo[1,2-a]pyridin-6-yl)-4-oxoquinazolin-3 (4H)-yl)piperidine-1-carboxylate (176 mg, 98%) as a solid. LCMS (ES, m/z): 495.8 [M+H]⁺.

### Synthesis of Compound 210

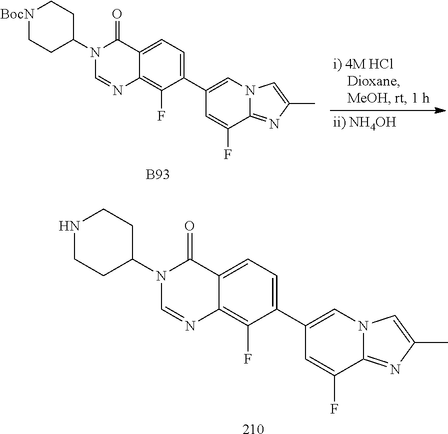 [SMILES: "B93" ; Cc1cn2cc(-c3ccc4c(=O)n(C5CCN(C(=O)OC(C)(C)C)CC5)cnc4c3F)cc(F)c2n1 ; Cc1cn2cc(-c3ccc4c(=O)n(C5CCNCC5)cnc4c3F)cc(F)c2n1 ; "210"]

To a solution of tert-butyl 4-(8-fluoro-7-(8-fluoro-2-methylimidazo[1,2-a]pyridin-6-yl)-4-oxoquinazolin-3(4H)-yl)piperidine-1-carboxylate 6 (172 mg, 0.35 mmol) in methanol (4.0 mL) was added 4 M HCl in dioxane (6.0 mL). The reaction mixture was stirred at room temperature for 1 h, then concentrated in vacuo to give a residue. The residue was purified by reverse phase chromatography using a gradient from 5 to 50% of acetonitrile in water containing 0.1% formic acid to afford a solid that was dissolved in water (2 mL), neutralized with 10% ammonium hydroxide (2 ml) and lyophilized to afford 8-fluoro-7-(8-fluoro-2-methylimidazo[1,2-a]pyridin-6-yl)-3-(piperidin-4-yl)quinazolin-4(3H)-one (101 mg, 74%). LCMS (ES, m/z): 395.8 [M+H]⁺. ¹H NMR (CH₂Cl₂-d₂, 400 MHz): δ_{H} 8.27 (2H, d, J=8.0 Hz), 8.14 (1H, d, J=8.4 Hz), 7.54-7.58 (2H, m), 7.17 (1H, d, J=11.6 Hz), 4.90 (1H, t, J=12.3 Hz), 3.24-3.29 (2H, m), 2.80-2.86 (2H, m), 2.49 (3H, s), 1.88-2.02 (4H, m).

### Example 41

### Synthesis of Compound 227

### Synthesis of Compound 227

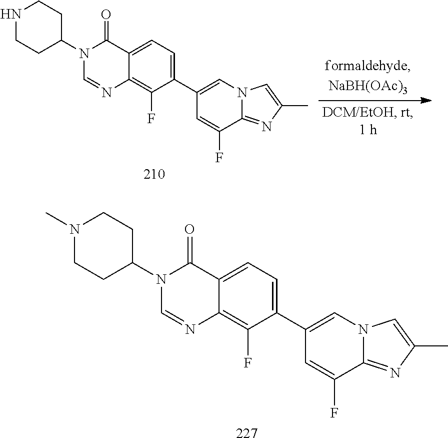

A mixture of 8-fluoro-7-(8-fluoro-2-methylimidazo[1,2-a]pyridin-6-yl)-3-(piperidin-4-yl)quinazolin-4(3H)-one (60 mg, 0.15 mmol) and formaldehyde (37% in water, 23 mg, 0.061 mL, 0.76 mmol) in DCM (6 mL) and ethanol (2 mL) was stirred at room temperature for 1 h. NaBH(OAc)₃ (193 mg, 0.91 mmol) was added and the reaction mixture was stirred at room temperature for an additional 1 h. The reaction mixture was diluted with DCM (50 mL) and washed with saturated NaHCO₃ (2×50 mL) and brine (50 mL). The organic layer was separated, dried over MgSO₄, filtered, and concentrated in vacuo to give a residue. The residue was purified by normal phase chromatography using a gradient from 10 to 50% (10% MeOH in EtOAc)/DCM with 1% Et3N additive to afford 8-fluoro-7-(8-fluoro-2-methylimidazo[1,2-a ]pyridin-6-yl)-3-(1-methylpiperidin-4-yl)quinazolin-4(3H)-one (25 mg, 40%) as a solid. LCMS (ES, m/z): 409.8 [M+H]⁺. ¹H NMR (CHCl₃-d, 400 MHz): δ_{H} 8.24 (2H, d, J=6.8 Hz), 8.17 (1H, d,=8.5 Hz), 7.56 (1H, t,=7.5 Hz), 7.50 (1H, s), 7.14 (1H, d,=11.1 Hz), 4.88 (1H, br s), 3.05 (2H, d, J=11.4 Hz), 2.52 (3H, s), 2.37 (3H, s), 2.23 (2H, s), 2.01 (4H, s).

### Example 42

### Synthesis of Compound 228

### Synthesis of Intermediate B94

Tert-butyl 4-(3-(8-fluoro-2-methylimidazo[1,2-a]pyridin-6-yl)-4-oxo-3,4-dihydroquinazolin-7-yl)piperazine-1-carboxylate was prepared using the procedure described in Example 3, where 2-methyl-2H-indazol-5-amine was substituted for 8-fluoro-2-methylimidazo[1,2-a]pyridin-6-amine in step 1, and steps 2 and 3 of Example 3 were subsequently applied, substituting 1-methylpiperazine for tert-butyl piperazine-1-carboxylate in step 3. Tert-butyl 4-(3-(8-fluoro methylimidazo[1,2-a]pyridin-6-yl)-4-oxo-3,4-dihydroquinazolin-7-yl)piperazine-1-carboxylate was obtained as a solid. LCMS (ES, m/z): 479.2 [M+H]⁺.

### Synthesis of Compound 228

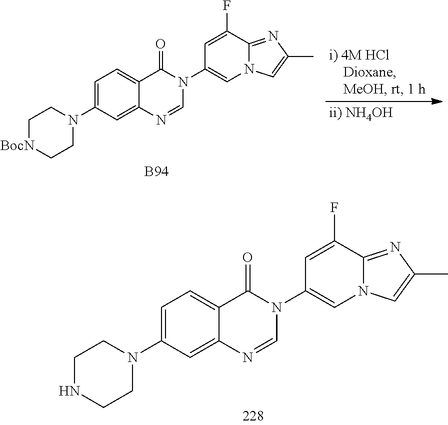 [SMILES: "B94" ; Cc1cn2cc(-n3cnc4cc(N5CCN(C(=O)OC(C)(C)C)CC5)ccc4c3=O)cc(F)c2n1 ; Cc1cn2cc(-n3cnc4cc(N5CCNCC5)ccc4c3=O)cc(F)c2n1]

To a solution of tert-butyl 4-(3-(8-fluoro-2-methylimidazo[1,2-a]pyridin-6-yl)-4-oxo-3,4-dihydroquinazolin-7-yl)piperazine-1-carboxylate (50 mg, 0.10 mmol) in methanol (2.0 mL) was added 4 M HCl in dioxane (2.0 mL). The reaction mixture was stirred at room temperature for 1 h, then concentrated in vacuo to give a residue. The residue was purified by reverse phase chromatography using a gradient from 5 to 50% of acetonitrile in water containing 0.1% formic acid to afford a white solid that was dissolved in water (2 mL), neutralized with 10% ammonium hydroxide (1 ml), and lyophilized to afford 3-(8-fluoro-2-methylimidazo[1,2-a]pyridin-6-yl)-7-(piperazin-1-yl)quinazolin-4(3H)-one (27 mg, 68%). LCMS (ES, m/z): 378.8 [M+H]⁺. ¹H NMR (CH₃OH-d₄, 400 MHz)*: δ_{H} 8.51 (1H, s), 8.23 (1H, s), 8.12 (1H, d, J=9.0 Hz), 7.76 (1H, s), 7.19-7.26 (2H, m), 7.06 (1H, s), 3.52 (4H, m), 3.12 (4H, m), 2.46 (3H, s).

### Example 43

### Synthesis of Compound 229

### Synthesis of Compound 229

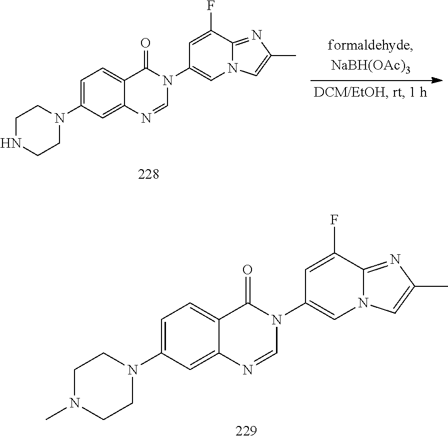

A mixture of 3-(8-fluoro-2-methylimidazo[1,2-c]pyridin-6-yl)-7-(piperazin-1-yl)quinazolin-4(3H)-one (22 mg, 0.06 mmol) and formaldehyde (37% in water, 9 mg, 0.024 mL, 0.29 mmol) in DCM (6 mL) and ethanol (2 mL) was stirred at room temperature for 1 h. NaBH(OAc)₃ (74 mg, 0.35 mmol) was added, and the reaction mixture was stirred at room temperature for an additional 1 h. The reaction mixture was diluted with DCM (50 mL) and washed with saturated NaHCO₃ (2×50 mL) and brine (50 mL). The organic layer was separated, dried over MgSO₄, filtered, and concentrated in vacuo to give a residue. The residue was purified by normal phase chromatography using a gradient from 10 to 50% (EtOAc/10% MeOH)/DCM with 1% Et₃N additive to afford 3-(8-fluoro-2-methylimidazo[1,2-a]pyridin-6-yl)-7-(4-methylpiperazin-1-yl)quinazolin-4(3H)-one (5.5 mg, 24%) as a solid. LCMS (ES, m/z): 392.9 [M+H]⁺. ¹H NMR (CH₂Cl₂-d₂, 400 MHz): δ_{H} 8.12 (2H, d+S, J=9.8 Hz), 8.01 (1H, s), 7.55 (1H, s), 7.13 (1H, d, J=9.1 Hz), 7.04 (1H, s), 6.97 (1H, d, J=10.7 Hz), 3.46 (4H, bs), 2.56 (4H, bs), 2.49 (3H, s), 2.34 (3H, s).

### Example 44

### Synthesis of Compound 230

### Synthesis of Intermediate B95

Tert-butyl 4-(5-fluoro-7-(8-fluoro-2-methylimidazo[1,2-c]pyridin-6-yl)-4-oxoquinazolin-3 (4H)-yl)piperidine-1-carboxylate was prepared according to the procedure described in Example 61, substituting 2-amino-5-bromo-6-fluorobenzoic acid for 2-amino-5-bromo-3-fluorobenzoic acid in step 1, and subsequently applying steps 2 and step 3 of Example 61. Tert-butyl 4-(5-fluoro-7-(8-fluoro-2-methylimidazo[1,2-c]pyridin-6-yl)-4-oxoquinazolin-3(4H)-yl)piperidine-1-carboxylate was obtained as a solid. LCMS (ES, m/z): 495.8 [M+H]⁺.

### Synthesis of Compound 230

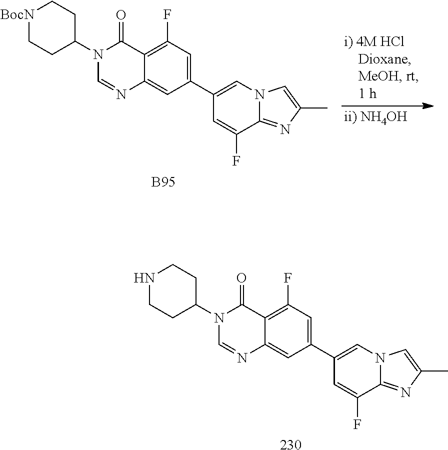 [SMILES: "B95" ; Cc1cn2cc(-c3cc(F)c4c(=O)n(C5CCN(C(=O)OC(C)(C)C)CC5)cnc4c3)cc(F)c2n1 ; Cc1cn2cc(-c3cc(F)c4c(=O)n(C5CCNCC5)cnc4c3)cc(F)c2n1 ; "230"]

To a solution of tert-butyl 4-(5-fluoro-7-(8-fluoro-2-methylimidazo[1,2-a]pyridin-6-yl)-4-oxoquinazolin-3(4H)-yl)piperidine-1-carboxylate (75 mg, 0.15 mmol) in methanol (2.0 mL) was added 4 M HCl in dioxane (2.0 mL). The reaction mixture was stirred at room temperature for 1 h, then concentrated in vacuo to give a residue. The residue was purified by reverse phase chromatography using a gradient from 5 to 50% of acetonitrile in water containing 0.1% formic acid to give a solid that was dissolved in water (2 mL), neutralized with 10% ammonium hydroxide (1 ml), and lyophilized to afford 5-fluoro-7-(8-fluoro-2-methylimidazo[1,2-a]pyridin-6-yl)-3-(piperidin-4-yl)quinazolin-4(3H)-one (45 mg, 75%). LCMS (ES, m/z): 395.8 [M+H]⁺. ¹H NMR (CH₂Cl₂-d₂, 400 MHz)*: δ_{H} 8.41 (1H, s), 8.34 (1H, s), 7.74 (1H, s), 7.61 (1H, s), 7.42 (1H, d, J=11.7 Hz), 7.30 (1H, d, J=11.4 Hz), 4.98 (1H, m), 3.62 (2H, d, J=13.1 Hz), 3.15 (2H, t, J=13.0 Hz), 2.51-2.61 (2H, m), 2.47 (3H, s), 2.16 (2H, d, J=13.5 Hz).

### Example 45

### Synthesis of Compound 231

### Synthesis of Compound 231

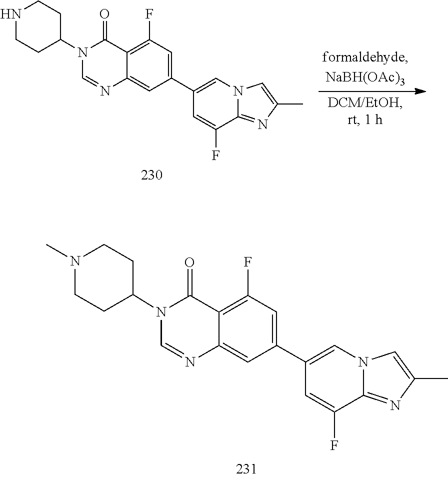

A mixture of 5-fluoro-7-(8-fluoro-2-methylimidazo[1,2-a]pyridin-6-yl)-3-(piperidin-4-yl)quinazolin-4(3H)-one (40 mg, 0.10 mmol), and formaldehyde (37% in water, 15 mg, 0.041 mL, 0.51 mmol) in DCM (6 mL) and ethanol (2 mL), was stirred at room temperature for 1 h. NaBH(OAc)₃ (129 mg, 0.61 mmol) was added, and the reaction mixture was stirred at room temperature for an additional 1 h. The reaction mixture was diluted with DCM (50 mL) and washed with saturated NaHCO₃ (2×50 mL) and brine (50 mL). The organic layer was separated, dried over MgSO₄, filtered, and concentrated in vacuo to give a residue. The residue was purified by normal phase chromatography using a gradient from 10 to 50% (10% MeOH in EtOAc)/DCM with 1% Et3N additive to afford 5-fluoro-7-(8-fluoro-2-methylimidazo[1,2-a ]pyridin-6-yl)-3-(1-methylpiperidin-4-yl)quinazolin-4(3H)-one (17 mg, 41%) as a solid. LCMS (ES, m/z): 409.8 [M+H]⁺. ¹H NMR (CH₂Cl₂-d₂, 400 MHz): δ_{H} 8.27 (1H, s), 8.18 (1H, s), 7.68 (1H, s), 7.55 (1H, d, J=2.9 Hz), 7.31-7.34 (1H, m), 7.20 (1H, d, J=11.5 Hz), 4.76-4.83 (1H, m), 3.01 (2H, d, J=11.5 Hz), 2.48 (3H, s), 2.32 (3H, s), 2.20 (2H, t, J=11.4 Hz), 1.96-2.05 (4H, m).

### Example 46

### Synthesis of Compound 232

### Synthesis of Intermediate B96

Tert-butyl 4-(7-(2,7-dimethyl-2H-indazol-5-yl)-5-fluoro-4-oxoquinazolin-3(4H)-yl)piperidine-1-carboxylate was prepared according to the procedure described in Example 61, substituting 2-amino-5-bromo-6-fluorobenzoic acid for 2-amino-5-bromo-3-fluorobenzoic acid in step 1, and subsequently applying steps 2 and step 3 of Example 61, substituting 2,7-dimethyl-5-(4,4,5,5-tetramethyl-1,3,2-dioxaborolan-2-yl)-2H-indazole for 8-fluoro-2-methyl-6-(4,4,5,5-tetramethyl-1,3,2-dioxaborolan-2-yl)imidazo[1,2-a]pyridine as the starting material in step 3. Tert-butyl 4-(7-(2,7-dimethyl-2H-indazol-5-yl)-5-fluoro-4-oxoquinazolin-3 (4H)-yl)piperidine-1-carboxylate was obtained as a solid. LCMS (ES, m/z): 492.2 [M+H]⁺.

### Synthesis of Compound 232

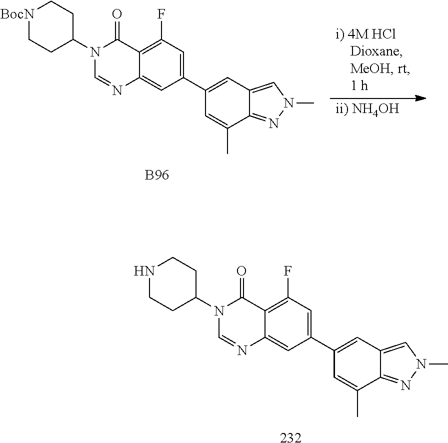 [SMILES: "B96" ; Cc1cc(-c2cc(F)c3c(=O)n(C4CCN(C(=O)OC(C)(C)C)CC4)cnc3c2)cc2cn(C)nc12 ; Cc1cc(-c2cc(F)c3c(=O)n(C4CCNCC4)cnc3c2)cc2cn(C)nc12 ; "232"]

To a solution of tert-butyl 4-(7-(2,7-dimethyl-2H-indazol-5-yl)-5-fluoro-4-oxoquinazolin-3 (4H)-yl)piperidine-1-carboxylate (70 mg, 0.14 mmol) in methanol (2.0 mL) was added 4 M HCl in dioxane (2.0 mL). The reaction mixture was stirred at room temperature for 1 h, then concentrated in vacuo to give a residue. The residue was purified by reverse phase chromatography using a gradient from 5 to 50% of acetonitrile in water containing 0.1% formic acid to afford a solid which was dissolved in water (2 mL), neutralized with 10% ammonium hydroxide (1 ml), and lyophilized to afford 7-(2,7-dimethyl-2H-indazol-5-yl)-5-fluoro-3-(piperidin-4-yl)quinazolin-4(3H)-one (42 mg, 75%). LCMS (ES, m/z): 391.8 [M+H]⁺. ¹H NMR (CHCl₃-d, 400 MHz): δ_{H} 8.15 (1H, s), 7.99 (1H, s), 7.79 (2H, d, J=14.1 Hz), 7.46 (1H, d, J=12.1 Hz), 7.38 (1H, s), 4.96-5.01 (1H, m), 4.27 (3H, s), 3.40 (2H, d, J=12.3 Hz), 2.88-2.95 (2H, m), 2.70 (3H, s), 1.97-2.08 (4H, m).

### Example 47

### Synthesis of Compound 233

### Synthesis of Compound 233

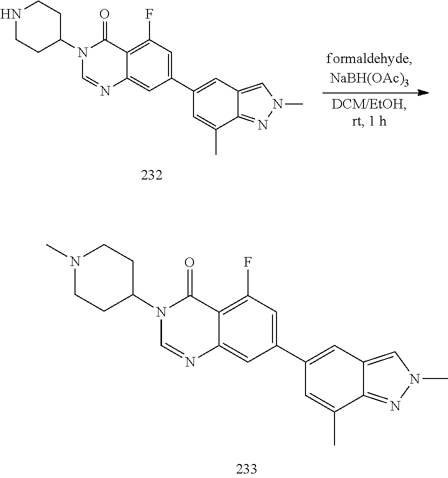

A mixture of 7-(2,7-dimethyl-2H-indazol-5-yl)-5-fluoro-3-(piperidin-4-yl)quinazolin-4(3H)-one (50 mg, 0.13 mmol) and formaldehyde (37% in water, 19 mg, 0.052 mL, 0.64 mmol) in DCM (6 mL) and ethanol (2 mL) was stirred at room temperature for 1 h. NaBH(OAc)₃ (162 mg, 0.76 mmol) was added, and the reaction mixture was stirred at room temperature for an additional 1 h.

The reaction mixture was diluted with DCM (50 mL) and washed with saturated aqueous NaHCO₃ (2×50 mL) and brine (50 mL), The organic layer was separated, dried over MgSO₄, filtered, and concentrated in vacuo to give a residue. The residue was purified by normal phase chromatography using a gradient from 10 to 50% (10% MeOH in EtOAc)/DCM with 1% Et3N additive to afford 7-(2,7-dimethyl-2H-indazol-5-yl)-5-fluoro-3-(1-methylpiperidin-4-yl)quinazolin-4(3H)-one (20 mg, 39%) as a solid. LCMS (ES, m/z): 405.8 [M+H]⁺. ¹H NMR (CHCl₃-d, 400 MHz): δ_{H} 8.14 (1H, s), 7.99 (1H, s), 7.80 (1H, s), 7.75 (1H, s), 7.45 (1H, d, J=12.1 Hz), 7.37 (1H, s), 4.85-4.93 (1H, m), 4.27 (3H, s), 3.02-3.05 (2H, m), 2.70 (3H, s), 2.37 (3H, s), 2.18-2.25 (2H, m), 1.99 (4H, bs).

### Example 48

### Synthesis of Compound 234

### Synthesis of Intermediate B97

Tert-butyl 4-(7-(2,7-dimethyl-2H-indazol-5-yl)-8-fluoro-4-oxoquinazolin-3(4H)-yl)piperidine-1-carboxylate was prepared according to the procedure described in Example 61, where 8-fluoro-2-methyl-6-(4,4,5,5-tetramethyl-1,3,2-dioxaborolan-2-yl)imidazo[1,2-a]pyridine was replaced with 2,7-dimethyl-5-(4,4,5,5-tetramethyl-1,3,2-dioxaborolan-2-yl)-2H-indazole in step 3. Tert-butyl 4-(7-(2,7-dimethyl-2H-indazol-5-yl)-8-fluoro-4-oxoquinazolin-3(4H)-yl)piperidine-1-carboxylate was obtained as a solid. LCMS (ES, m/z): 492.2 [M+H]⁺

### Synthesis of Compound 234

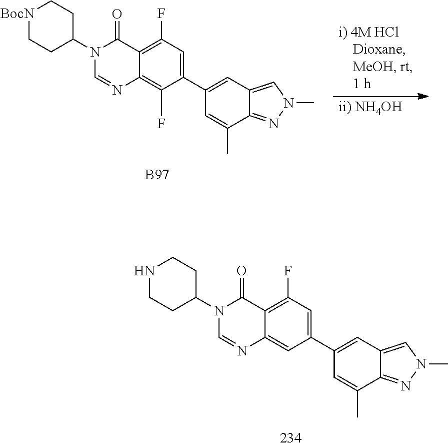 [SMILES: "B97" ; Cc1cc(-c2cc(F)c3c(=O)n(C4CCN(C(=O)OC(C)(C)C)CC4)cnc3c2F)cc2cn(C)nc12 ; Cc1cc(-c2cc(F)c3c(=O)n(C4CCNCC4)cnc3c2)cc2cn(C)nc12 ; "234"]

To a solution of tert-butyl 4-(7-(2,7-dimethyl-2H-indazol-5-yl)-8-fluoro-4-oxoquinazolin-3 (4H)-yl)piperidine-1-carboxylate (103 mg, 0.21 mmol) in methanol (4.0 mL) was added 4 M HCl in dioxane (4.0 mL). The reaction mixture was stirred at room temperature for 1 h, then concentrated in vacuo to give a residue. The residue was purified by reverse phase chromatography using a gradient from 5 to 50% of acetonitrile in water containing 0.1% formic acid to afford a solid that was dissolved in water (2 mL), neutralized with 10% ammonium hydroxide (1 ml), and lyophilized to afford 7-(2,7-dimethyl-2H-indazol-5-yl)-8-fluoro (piperidin-4-yl)quinazolin-4(3H)-one (70 mg, 85%). LCMS (ES, m/z): 391.9 [M+H]⁺. ¹H NMR (CHCl₃-d, 400 MHz): δ_{H} 8.24 (1H, s), 8.13 (1H, d, J=8.4 Hz), 7.98 (1H, s), 7.78 (1H, s), 7.63 (1H, t, J=7.5 Hz), 7.30 (1H, s), 4.97 (1H, t, J=12.2 Hz), 4.28 (3H, s), 3.30 (2H, d, J=12.3 Hz), 2.87 (2H, t, J=12.0 Hz), 2.70 (3H, s), 2.02 (2H, d, J=11.7 Hz), 1.85-1.94 (2H, m).

### Example 49

### Synthesis of Compound 235

### Synthesis of Compound 235

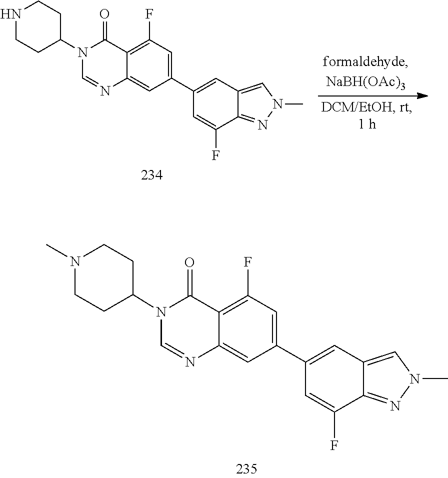

A mixture of 7-(2,7-dimethyl-2H-indazol-5-yl)-8-fluoro-3-(piperidin-4-yl)quinazolin-4(3H)-one (41 mg, 0.11 mmol) and formaldehyde (37% in water, 16 mg, 0.043 mL, 0.52 mmol) in DCM (6 mL) and ethanol (2 mL) was stirred at room temperature for 1 h. NaBH(OAc)₃ (133 mg, 0.63 mmol) was added, and the reaction mixture was stirred at room temperature for an additional 1 h. The reaction mixture was diluted with DCM (50 mL) and washed with saturated aqueous NaHCO₃ (2×50 mL) and brine (50 mL). The organic layer was separated, dried over MgSO₄, filtered, and concentrated in vacuo to give a residue. The residue was purified by normal phase chromatography using a gradient from 10 to 50% (10% MeOH in EtOAc)/DCM with 1% Et3N additive to afford 7-(2,7-dimethyl-2H-indazol-5-yl)-8-fluoro-3-(1-methylpiperidin-4-yl)quinazolin-4(3H)-one (6.5 mg, 15%) as a solid. LCMS (ES, m/z): 405.8 [M+H]⁺. ¹H NMR (CH₂Cl₂-d₂, 400 MHz): δ_{H} 8.24 (1H, s), 8.10 (1H, d, J=8.4 Hz), 8.02 (1H, s), 7.79 (1H, s), 7.62 (1H, t, J=7.6 Hz), 7.30 (1H, s), 4.76-4.83 (1H, m), 4.25 (3H, s), 3.01 (2H, d, J=11.2 Hz), 2.65 (3H, s), 2.33 (3H, s), 2.17-2.23 (2H, m), 2.02-2.10 (2H, m), 1.95-2.01 (2H, m).

### Example 50

### Synthesis of Compound 236

### Synthesis of Intermediate B98

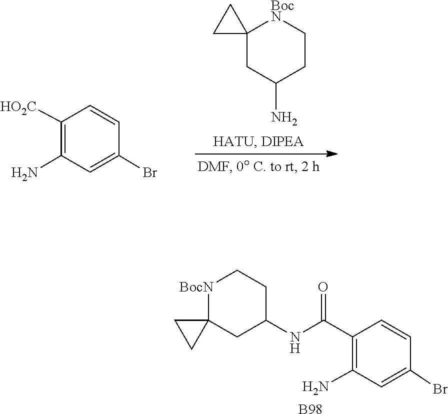

2-amino-4-bromobenzoic acid 1 (500 mg, 2.31 mmol) and tert-butyl 7-amino azaspiro[2.5]octane-4-carboxylate (573 mg, 2.41 mmol) were dissolved in DMF (11.6 mL) and cooled in an ice bath. To this solution was added DIPEA (1.2 mL, 6.94 mmol) dropwise, followed by HATU (968 mg, 2.55 mmol). The reaction mixture was stirred and allowed to warm to room temperature over 2 h, then diluted with ethyl acetate (50 mL), and washed with saturated aqueous NH₄Cl (30 mL), followed by saturated NaHCO₃ (30 mL), and brine (40 mL). The organic layer was dried over anhydrous Na₂SO₄, filtered, and concentrated in vacuo to afford tert-butyl 7-(2-amino-4-bromobenzamido)-4-azaspiro[2.5]octane-4-carboxylate (948 mg, 97%) as a solid. LCMS (ES, m/z): 446.1 [M+Na]⁺

### Synthesis of Intermediate B99

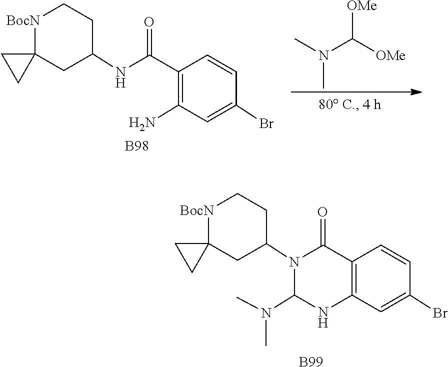

Tert-butyl 7-(2-amino-4-bromobenzamido)-4-azaspiro[2.5]octane-4-carboxylate (500 mg, 1.18 mmol) and N,N-Dimethylformamide dimethyl acetal (3.1 mL, 23.6 mmol) were combined and heated at 80° C. for 4 hours. The reaction mixture was diluted with ethyl acetate (50 mL) and washed with saturated NaHCO₃ (30 mL) and brine (2×30 mL). The organic phase was dried over Na₂SO₄, filtered, and concentrated in vacuo to give a residue. The residue was triturated with TBME (20 mL), the solid was collected by filtration and solvent traces were removed under reduced pressure to afford tert-butyl 7-(7-bromo-2-(dimethylamino)-4-oxo-1,4-dihydroquinazolin-3(2H)-yl)-4-azaspiro[2.5]octane-4-carboxylate (466 mg, 83%) as a solid. LCMS (ES, m/z): 479.2 [M+H]⁺. s

Synthesis of Compound 236

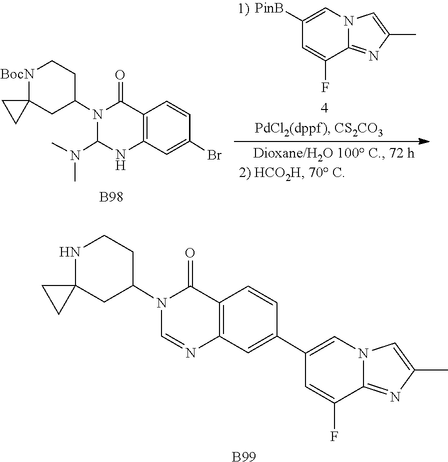

Tert-butyl 7-(7-bromo-2-(dimethylamino)-4-oxo-1,4-dihydroquinazolin-3(2H)-yl) azaspiro[2.5]octane-4-carboxylate (120 mg, 0.25 mmol), 8-fluoro-2-methyl-6-(4,4,5,5-tetramethyl-1,3,2-dioxaborolan-2-yl)imidazo[1,2-a]pyridine (83 mg, 0.30 mmol), PdCl₂(dppf) (18 mg, 0.025 mmol), and Cs₂CO₃ (408 mg, 1.25 mmol) were dissolved in dioxane (2.8 mL) and H₂O (280 μL) and heated at 100° C. for 72 hours. The reaction mixture was diluted with ethyl acetate (40 mL) and washed with saturated NaHCO₃ (25 mL) and brine (2×25 mL). The organic phase was dried over Na₂SO₄, filtered, and concentrated in vacuo to give a residue. The residue was purified by flash chromatography on a silica gel column using a gradient of 0-20% MeOH in DCM. Selected fractions were combined and evaporated in vacuo to give a solid. To the solid was added neat formic acid (3 mL) and the reaction mixture was stirred vigorously at 70° C. for 2 h, then concentrated in vacuo to give a residue and the residue dissolved in water (6 mL). The aqueous phase was washed with DCM (2×5 mL) and neutralized with (NH₄)₂CO₃ to form a suspension. The resulting suspension was cooled down to 4° C. and the precipitate was collected by vacuum filtration. The solid was washed with cold water and dried under high vacuum at room temperature overnight to afford 7-(8-fluoro-2-methylimidazo[1,2-a]pyridin-6-yl)-3-(4-azaspiro[2.5]octan-7-yl)quinazolin-4(3H)-one (36 mg, 36%) as a solid. LCMS (ES, m/z): 404.2 [M+H]⁺. ¹H NMR (DMSO-d₆, 300 MHz): δ 9.00 (1H, s), 8.48 (1H, s), 8.22 (1H, d, J=8.4 Hz), 8.03 (1H, s), 7.91 (1H, d, J=8.8 Hz), 7.85 (1H, s), 7.68 (1H, d, J=12.6 Hz), 4.90 (1H, m), 3.04 (1H, d, J=13.4 Hz), 2.72 (1H, m), 2.39 (3H, s), 1.85-1.98 (2H, br m), 1.23 (1H, d, J=12.2 Hz), 0.60 (1H, m), 0.45 (4H, m).

### Example 51

### Synthesis of Compound 237

### Synthesis of Intermediate B100

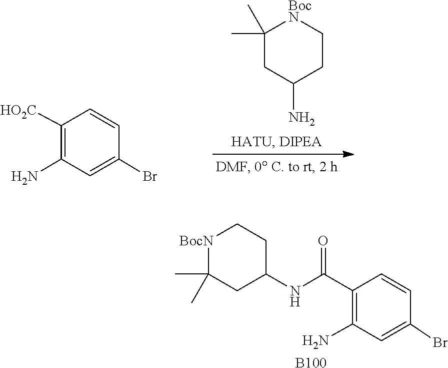

2-amino-4-bromobenzoic acid (500 mg, 2.31 mmol) and tert-butyl 4-amino-2,2-dimethylpiperidine-1-carboxylate (577 mg, 2.50 mmol) were dissolved in DMF (11.6 mL) and cooled in an ice bath. To this solution was added DIPEA (1.2 mL, 6.94 mmol) dropwise, followed by HATU (968 mg, 2.55 mmol). The reaction mixture was stirred and allowed to warm to room temperature over 2 h. The reaction mixture was diluted with ethyl acetate (50 mL) and washed with saturated aqueous NH₄Cl (30 mL), followed by saturated NaHCO₃ (30 mL), and brine (40 mL). The organic layer was dried over anhydrous Na₂SO₄, filtered and concentrated in vacuo to afford tert-butyl 4-(2-amino-4-bromobenzamido)-2,2-dimethylpiperidine-1-carboxylate (961 mg, 97%) as a solid. LCMS (ES, m/z): 448.1 [M+Na]⁺.

### Synthesis of Intermediate B101

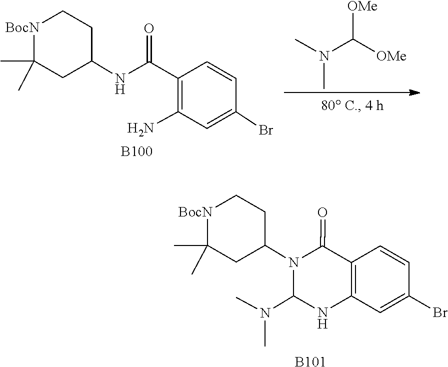

Tert-butyl 4-(2-amino-4-bromobenzamido)-2,2-dimethylpiperidine-1-carboxylate (500 mg, 1.18 mmol) and N,N-Dimethylformamide dimethyl acetal (3.1 mL, 23.6 mmol) were combined in a sealed tube and heated at 80° C. for 4 h. The reaction mixture was diluted with ethyl acetate (50 mL) and washed with saturated NaHCO₃ (30 mL) and brine (2×30 mL). The organic phase was dried over Na₂SO₄, filtered, and concentrated in vacuo to give a residue. The residue was triturated with TBME (20 mL), the solid was collected by filtration and solvent traces were removed under reduced pressure to afford tert-butyl 4-(7-bromo-2-(dimethylamino)-4-oxo-1,4-dihydroquinazolin-3(2H)-yl)-2,2-dimethylpiperidine-1-carboxylate (463 mg, 82%) as a solid. LCMS (ES, m/z): 481.2 [M+H]⁺.

### Synthesis of Compound 237

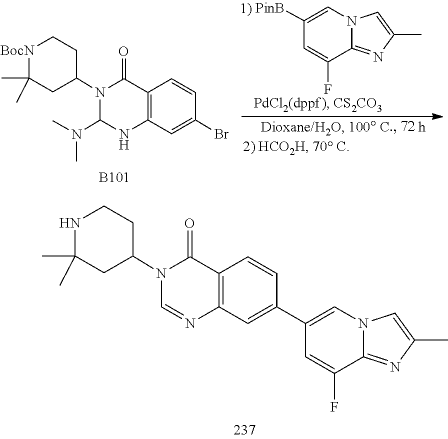

Tert-butyl 4-(7-bromo-2-(dimethylamino)-4-oxo-1,4-dihydroquinazolin-3(2H)-yl)-2,2-dimethylpiperidine-1-carboxylate (120 mg, 0.25 mmol), 8-fluoro-2-methyl-6-(4,4,5,5-tetramethyl-1,3,2-dioxaborolan-2-yl)imidazo[1,2-a]pyridine (83 mg, 0.30 mmol), PdCl₂(dppf) (18 mg, 0.025 mmol), and Cs₂CO₃ (408 mg, 1.25 mmol) were dissolved in dioxane (2.8 mL) and H₂O (280 μL) and heated at 100° C. for 72 h. The reaction mixture was diluted with ethyl acetate (40 mL) and washed with saturated NaHCO₃ (25 mL) and brine (2×25 mL). The organic phase was dried over Na₂SO₄, filtered, and concentrated in vacuo to give a residue. The residue was purified by flash chromatography on a silica gel column using a gradient of 0-20% MeOH in DCM. Selected fractions were combined and evaporated in vacuo to give a solid. To the solid was added neat formic acid (3 mL), and the reaction mixture was stirred vigorously at 70° C. for 2 h. The reaction mixture was concentrated in vacuo to give a residue and the residue was purified by flash chromatography on a C18 column using a gradient of 5-70% MeCN in water with 0.1% formic acid additive. Selected fractions were combined, neutralized with (NH₄)₂CO_{3,} and lyophilized to afford 3-(2,2-dimethylpiperidin-4-yl)-7-(8-fluoro-2-methylimidazo[1,2-a]pyridin-6-yl)quinazolin-4(3H)-one (49 mg, 48%) as a solid. LCMS (ES, m/z): 406.2 [M+H]⁺. ¹H NMR (DMSO-d₆, 300 MHz): δ 9.00 (1H, d, J=1.5 Hz), 8.46 (1H, s), 8.27 (1H, s), 8.23 (1H, d, J=8.4 Hz), 8.04 (1H, d, J=1.8 Hz), 7.92 (1H, dd, J=8.4, 1.9 Hz), 7.86 (1H, d, J=3.1 Hz), 7.69 (1H, d, J=12.6 Hz), 4.98 (1H, m), 3.01 (2H, m), 2.39 (3H, s), 1.76-2.01 (4H, br m), 1.23 (3H, s), 1.18 (3H, s).

### Example 52

### Synthesis of Compound 188

### Synthesis of Intermediate B102

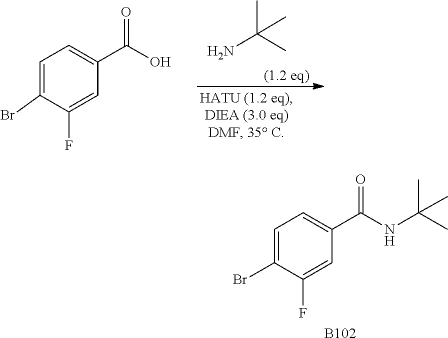

A mixture of 4-bromo-3-fluorobenzoic acid (1.0g, 4.56 mmol, 1.00 equiv), DMF (20.0 mL), 2-methylpropan-2-amine (0.4g, 5.48 mmol, 1.20 equiv), HATU (2.1g, 5.45 mmol, 1.20 equiv), and DIEA (1.7 g, 13.69 mmol, 3.00 equiv) was stirred for 4 h at 35° C. The reaction mixture was quenched with water (40 mL). The resulting solution was extracted with ethyl acetate (3×40 mL), and the organic layers combined. The resulting mixture was washed with 1/2 saturated aqueous NaCl (3 ×100 mL) and saturated aqueous NaCl (1 ×100 mL). The organic layer was dried over anhydrous sodium sulfate, filtered, and the filtrate concentrated under vacuum to give a residue. The residue was purified by silica gel column chromatography, eluted with ethyl acetate/petroleum ether to afford 4-bromo-N-tert-butyl-3-fluorobenzamide as a solid (1.0 g, 79.8%). LCMS (ES, m/z): 274 [M+H]⁺.

### Synthesis of Intermediate B103

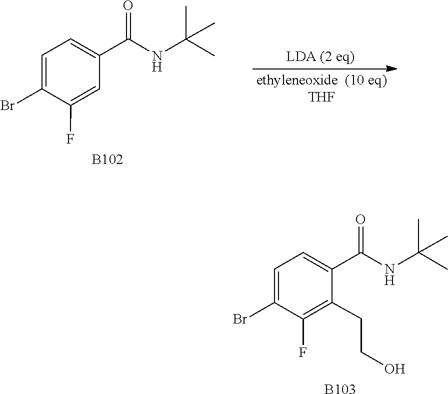

A solution of 4-bromo-N-tert-butyl-3-fluorobenzamide (900.0 mg, 3.28 mmol, 1.00 equiv) in THF (18.00 mL) was maintained under a nitrogen atmosphere. To the solution was added lithiobis(propan-2-yl)amine (703.3 mg, 6.56 mmol, 2.00 equiv) dropwise with stirring at −60 ° C. The reaction mixture was stirred for 30 min at −45° C. To the reaction mixture was added ethyleneoxide (1446.2 mg, 32.83 mmol, 10.00 equiv), while stirring at 0° C.The resulting solution was stirred for 1 h at room temperature, then quenched with 1/2 saturated aqueous NaCl (50 mL). The resulting solution was extracted with ethyl acetate (3×50 mL), and the organic layers combined. The resulting mixture was washed with saturated aqueous NaCl (1 ×150 mL). The organic layer was dried over anhydrous sodium sulfate, filtered, and the filtrate concentrated under vacuum to give a residue. The residue was purified by silica gel column chromatography, eluted with ethyl acetate/petroleum ether to afford 4-bromo-N-tert-butyl-3-fluoro-2-(2-hydroxyethyl)benzamide (800 mg, 76.5%) as a solid. LCMS (ES, m/z): 318 [M+H]⁺.

### Synthesis of Intermediate B104

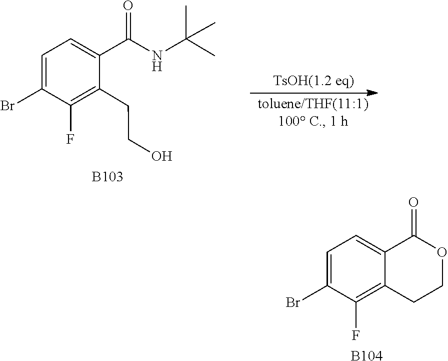

A mixture of 4-bromo-N-tert-butyl-3-fluoro-2-(2-hydroxyethyl) benzamide (700.0 mg, 2.20 mmol, 1.00 equiv), toluene (35.00 mL), THF (3.50 mL), TsOH (454.6 mg, 2.64 mmol, 1.20 equiv) was stirred for 1 h at 100° C. The reaction mixture was concentrated under vacuum, then quenched with 1/2 saturated aqueous NaCl (50 mL). The resulting solution was extracted with ethyl acetate (3×50 mL), and the organic layers combined. The combined organic layers were washed with saturated aqueous NaCl (1×150 mL), dried over anhydrous sodium sulfate, and filtered. The filtrate was concentrated under vacuum to give a residue. The residue was purified by silica gel column chromatography, eluted with ethyl acetate/petroleum ether to afford 6-bromo-5-fluoro-3,4-dihydro-2-benzopyran-1-one (530 mg, 98.3%) as a solid. LCMS (ES, m/z): 245 [M+H]⁺.

### Synthesis of Intermediate B105

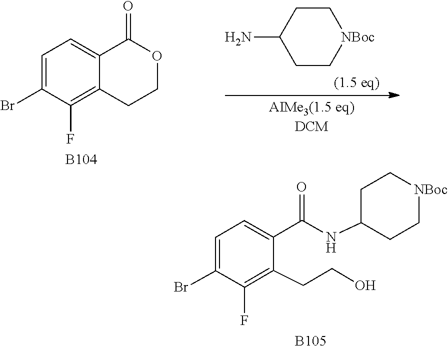

To tert-butyl 4-aminopiperidine-1-carboxylate (527.1 mg, 2.63 mmol, 1.50 equiv) in DCM (5.00 mL) was added AlMe₃ (189.7 mg, 2.63 mmol, 1.50 equiv) dropwise at 0° C. with stirring under a nitrogen atmosphere. The resulting solution was stirred for 30 min at room temperature. To the reaction mixture was added 6-bromo-5-fluoro-3,4-dihydro-2-benzopyran-1-one (430.0 mg, 1.75 mmol, 1.00 equiv), with stirring at room temperature. The resulting solution was stirred for 30 min at room temperature and for an additional 1 h at 40° C. The reaction mixture was quenched with water. The resulting solution was extracted with ethyl acetate (3×50 mL), and the organic layers combined. The resulting mixture was washed with saturated aqueous NaCl (1×150 mL), dried over anhydrous sodium sulfate, and filtered. The resulting mixture was concentrated under vacuum to give a reisude. The residue was purified by silica gel column chromatography, eluted with ethyl acetate/petroleum ether to afford tert-butyl 4-[4-bromo-3-fluoro-2-(2-hydroxyethyl) benzamido]piperidine-1-carboxylate (750 mg, 78.1%) as a solid. LCMS (ES, m/z): 445 [M+H]⁺.

### Synthesis of Intermediate B106

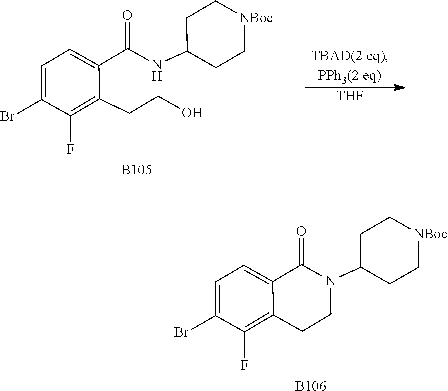

To a mixture of tert-butyl 4-[4-bromo-3-fluoro-2-(2-hydroxyethyl)benzamido]piperidine-1-carboxylate (700.0 mg, 1.57 mmol, 1.00 equiv), THF (70 mL), and PPh₃ (824.5 mg, 3.14 mmol, 2.00 equiv) was added TBAD (723.8 mg, 3.14 mmol, 2.00 equiv) dropwise while stirring at 0° C. The reaction mixture was stirred for 2 h at room temperature, then quenched with water. The resulting mixture was extracted with ethyl acetate (3×100 mL). The combined organic layers were washed with saturated aqueous NaCl (300 mL), dried over anhydrous sodium sulfate, and filtered. After filtration, the filtrate was concentrated under reduced pressure to give a reside. The residue was purified by silica gel column chromatography, eluted with ethyl acetate/petroleum ether to afford tert-butyl 4-(6-bromo-5-fluoro-1-oxo-3,4-dihydroisoquinolin-2-yl)piperidine-1-carboxylate (600 mg, 89.3%) as a solid. LCMS (ES, m/z): 427 [M+H]⁺.

### Synthesis of Intermediate B107

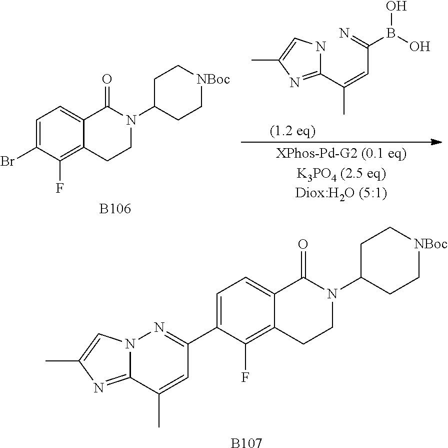

A mixture of tert-butyl 4-(6-bromo-5-fluoro-1-oxo-3,4-dihydroisoquinolin-2-yl)piperidine-1-carboxylate (80.0 mg, 0.18 mmol, 1.00 equiv), 2,8-dimethylimidazo[1,2-b]pyridazin-6-ylboronic acid (42.91 mg, 0.224 mmol, 1.2 equiv), K₃PO₄(aq) (119.2 mg, 0.56 mmol, 3.00 equiv), H₂O (0.8 mL, 44.407 mmol, 237.20 equiv)), dioxane (4 mL, 47.216 mmol, 252.20 equiv) and 2nd Generation XPhos precatalyst (14.73 mg, 0.019 mmol, 0.1 equiv)\ was stirred for 6 hat 80° C. The reaction mixture was quenched with water (20 mL), then extracted with ethyl acetate (3×20 mL). The combined organic layers were washed with saturated NaCl (50 mL), dried over anhydrous sodium sulfate, and filtered. After filtration, the filtrate was concentrated under reduced pressure to give a residue. The residue was purified by silica gel column chromatography, eluted with ethyl acetate/petroleum ether to afford tert-butyl 4-(6-{2,8-dimethylimidazo[1,2-b]pyridazin-6-yl}-5-fluoro-1-oxo-3,4-dihydroisoquinolin-2-yl)piperidine-1-carboxylate (60 mg, 64.93%) as an oil. LCMS (ES, m/z): 494 [M+H]⁺.

### Synthesis of Compound 188

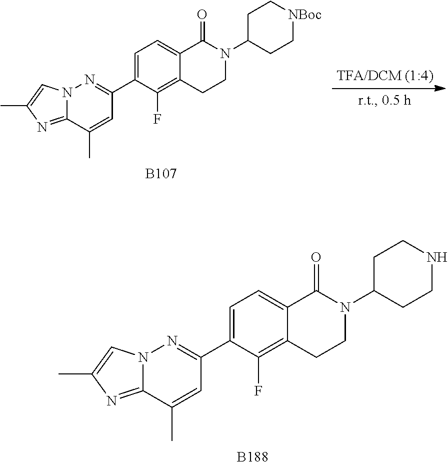 [SMILES: "B107" ; "B188" ; Cc1cn2nc(-c3ccc4c(c3F)CCN(C3CCN(C(=O)OC(C)(C)C)CC3)C4=O)cc(C)c2n1 ; Cc1cn2nc(-c3ccc4c(c3F)CCN(C3CCNCC3)C4=O)cc(C)c2n1]

A mixture of tert-butyl-4-(6-{2,8-dimethylimidazo[1,2-b]pyridazin-6-yl}-5-fluoro-1-oxo-3,4-dihydroisoquinolin-2-yl) piperidine-1-carboxylate (50 mg, 0.101 mmol, 1.00 equiv), DCM (2.0 mL), and TFA(0.5 mL) was stirred for 30 min at room temperature under a nitrogen atmosphere. The resulting mixture was concentrated under vacuum to give a residue. The residue was purified by Chiral-Prep-HPLC (Column, Xselect CSH OBD Column 30*150 mm 5 um, n; Mobile Phase A, water (10 mmol/L NH₄HCO₃), Mobile Phase B, CAN; Gradient 5% B up to 45% B in 8 min) to afford 6-{2,8-dimethylimidazo[1,2-b]pyridazin-6-yl}-5-fluoro-2-(piperidin-4-yl)-3,4-dihydroisoquinolin-1-one (21.8 mg, 54.37%) as a solid. LCMS (ES, m/z): 394 [M+H]⁺. ¹H-NMR (400 MHz, DMSO-d₆) δ 8.11 (s, 1H), 7.86 (d, J=8.1 Hz, 1H), 7.75 (t, J=7.7 Hz, 1H), 7.38 (dd, J=2.3, 1.2 Hz, 1H), 4.59 — 4.48 (m, 1H), 3.53 (t, J=6.5 Hz, 2H), 3.02 (q, J=7.1 Hz, 4H), 2.61 (d, J=1.1 Hz, 3H), 2.59 — 2.54 (m, 2H), 2.42 (s, 3H), 1.63 (qd, J=11.8, 3.9 Hz, 2H), 1.57 — 1.49 (m, 2H).

### Example 53

### Synthesis of Compounds 267-270, 281, and 282

### Synthesis of Intermediate B108

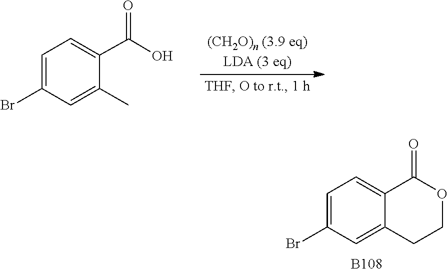

To 4-bromo-2-methylbenzoic acid (49.0 g, 227.85 mmol, 1.00 equiv) in THF (500 mL) was added LDA (24.4 g, 227.85 mmol, 1 equiv) dropwise at −40° C. under nitrogen atmosphere. The resulting mixture was stirred for 30 min at −40° C. under nitrogen atmosphere, then paraformaldehyde (82.1 g, 911.43 mmol, 4 equiv) was added dropwise at 15° C. under nitrogen atmosphere. The resulting mixture was stirred for 1 h at room temperature under nitrogen atmosphere. The reaction mixture was quenched with 3N HCl (500 mL) at 0° C., and extracted with ethyl acetate (3×500 mL). The combined organic layers were washed with brine (1×1000 mL), dried over anhydrous Na₂SO₄, and filtered. After filtration, the filtrate was concentrated under reduced pressure to give a residue. The residue was purified by silica gel column chromatography, eluted with PE/EA (10:01) to afford 6-bromo-3,4-dihydro-2-benzopyran-1-one (5.5 g, 10.6%) as a solid. LCMS (ES, m/z): 227 [M+H]⁺.

### Synthesis of Intermediate B109

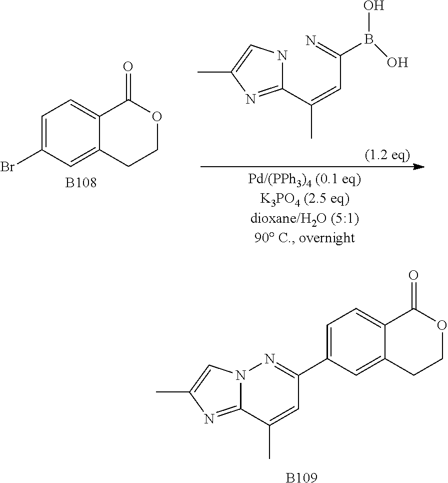

A solution of 6-bromo-3,4-dihydro-2-benzopyran-1-one (4.1g, 18.05 mmol, 1.00 equiv), 2,8-dimethylimidazo[1,2-b]pyridazin-6-ylboronic acid (4.1 g, 21.66 mmol, 1.2 equiv), Pd(PPh₃)₄ (2.1 g, 1.81 mmol, 0.1 equiv), and K₃PO₄ (9.6 g, 45.14 mmol, 2.5 equiv) in dioxane (40 mL) and water (8 mL) was stirred overnight at 90° C. under nitrogen atmosphere. The resulting mixture was diluted with water (100 mL), then extracted with ethyl acetate (3×100 mL). The combined organic layers were washed with brine (1×200 mL), dried over anhydrous Na₂SO₄, and filtered. After filtration, the filtrate was concentrated under reduced pressure to give a residue. The residue was purified by silica gel column chromatography, eluted with PE/EA (10:01) to afford 6-{2,8-dimethylimidazo[1,2-b]pyridazin-6-yl}-3,4-dihydro-2-benzopyran-1-one (3.0 g, 56.6%) as a solid. LCMS (ES, m/z): 294 [M+H]⁺.

### Synthesis of Intermediate B110

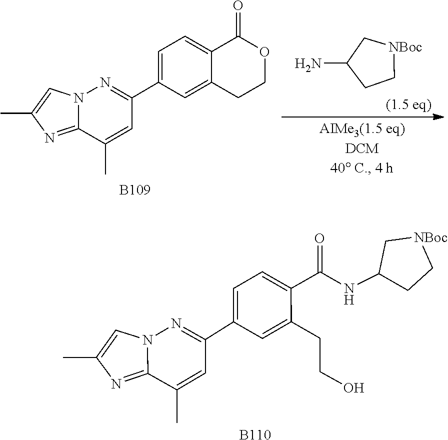 [SMILES: "B109" ; "B110" ; Cc1cn2nc(-c3ccc4c(c3)CCOC4=O)cc(C)c2n1 ; Cc1cn2nc(-c3ccc(C(=O)NC4CCN(C(=O)OC(C)(C)C)C4)c(CCO)c3)cc(C)c2n1 ; CC(C)(C)OC(=O)N1CCC(N)C1]

To a stirred solution of tert-butyl 3-aminopyrrolidine-1-carboxylate (139.4 mg, 0.75 mmol, 1.1 equiv) in DCM (4 mL) was added AlMe₃ (24.6 mg, 0.34 mmol, 0.5 equiv) dropwise at 0° C. under nitrogen atmosphere. To the above mixture was added 6-{2,8-dimethylimidazo[1,2-b]pyridazin-6-yl}-3,4-dihydro-2-benzopyran-1-one (200.0 mg, 0.68 mmol, 1.00 equiv) dropwise at 0° C. The resulting mixture was stirred for an additional 4 h at 40° C. The resulting mixture was diluted with water (40.0 mL), then extracted with CH₂Cl₂ (3×40 mL). The combined organic layers were washed with brine (1×40 mL), dried over anhydrous Na₂SO₄, and filtered. After filtration, the filtrate was concentrated under reduced pressure to give a residue. The residue was purified by silica gel column chromatography, eluted with CH₂Cl₂/MeOH (96:04) to afford tert-butyl 3-(4-{2,8-dimethylimidazo[1,2-b]pyridazin-6-yl}-2-(2-hydroxyethyl)benzamido)pyrrolidine-1-carboxylate (230.0 mg, 70.3%) as a solid. LCMS (ES, m/z): 480 [M+H]⁺.

### Synthesis of Intermediate B111

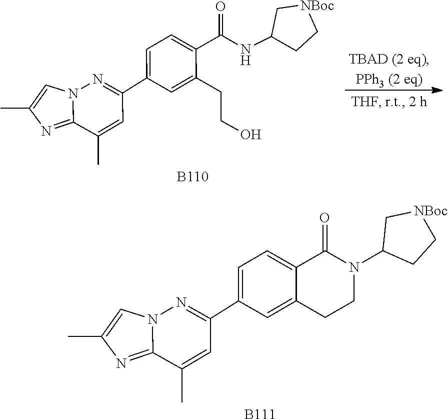 [SMILES: "B110" ; "B111" ; Cc1cn2nc(-c3ccc(C(=O)NC4CCN(C(=O)OC(C)(C)C)C4)c(CCO)c3)cc(C)c2n1 ; Cc1cn2nc(-c3ccc4c(c3)CCN(C3CCN(C(=O)OC(C)(C)C)C3)C4=O)cc(C)c2n1]

To a stirred solution of tert-butyl 3-(4-{2,8-dimethylimidazo[1,2-b]pyridazin-6-yl}-2-(2-hydroxyethyl)-benzamido)pyrrolidine-1-carboxylate (230.0 mg, 0.48 mmol, 1.00 equiv) and PPh₃ (251.6 mg, 0.96 mmol, 2 equiv) in THF (25 mL) was added TBAD (220.6 mg, 0.96 mmol, 2 equiv) dropwise at 0° C. under nitrogen atmosphere. The resulting mixture was stirred for 2 h at room temperature under nitrogen atmosphere. The resulting mixture was extracted with CH₂Cl₂ (3×30 mL). The combined organic layers were washed with brine (1×30 mL), dried over anhydrous Na₂SO₄, and filtered. After filtration, the filtrate was purified by silica gel column chromatography, eluted with PE/EA (0 :1) to afford tert-butyl 3-(6-{2,8-dimethylimidazo[1,2-b]pyridazin-6-yl}-1-oxo-3,4-dihydroisoquinolin-2-yl)pyrrolidine-1-carboxylate (120.0 mg, 54.21%) as a solid. LCMS (ES, m/z): 462 [M+H]⁺.

### Synthesis of Compound 269

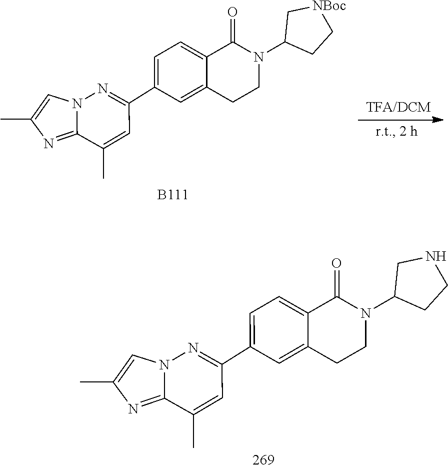 [SMILES: "B111" ; Cc1cn2nc(-c3ccc4c(c3)CCN(C3CCN(C(=O)OC(C)(C)C)C3)C4=O)cc(C)c2n1 ; Cc1cn2nc(-c3ccc4c(c3)CCN(C3CCNC3)C4=O)cc(C)c2n1 ; "269"]

A solution of tert-butyl 3-(6-{2,8-dimethylimidazo[1,2-b]pyridazin-6-yl}-1-oxo-3,4-dihydroisoquinolin-2-yl)pyrrolidine-1-carboxylate (120.0 mg, 0.26 mmol, 1.00 equiv) in TFA (0.75 mL) was stirred for 1 h at room temperature. The resulting mixture was concentrated under vacuum to afford 6-{2,8-dimethylimidazo[1,2-b]pyridazin-6-yl}-2-(pyrrolidin-3-yl)-3,4-dihydroisoquinolin-1-one (150 mg) as an oil. The crude product (75 mg) was purified by Prep-HPLC (Column:)(Bridge Prep OBD C18 Column, 30*150 mm, Sum; Mobile Phase A: Water (10 mmol/L NH₄HCO₃), Mobile Phase B: ACN; Flow rate: 60 mL/min; Gradient: 5% B to 35% B in 8 min) to afford 6-{2,8-dimethylimidazo[1,2-b]pyridazin-6-yl}-2-(pyrrolidin-3-yl)-3,4-dihydroisoquinolin-1-one (12.2 mg, 12.9%) as a solid.

### Synthesis of Compound 282

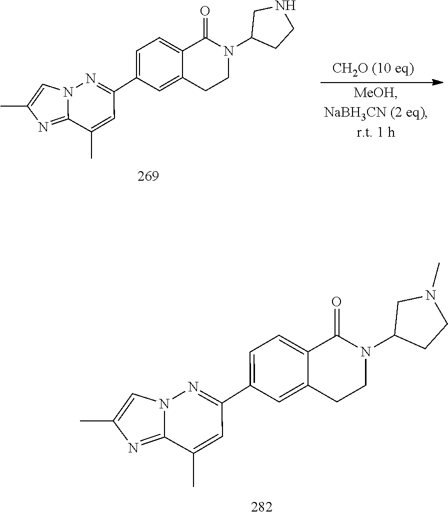 [SMILES: Cc1cn2nc(-c3ccc4c(c3)CCN(C3CCNC3)C4=O)cc(C)c2n1 ; "269" ; Cc1cn2nc(-c3ccc4c(c3)CCN(C3CCN(C)C3)C4=O)cc(C)c2n1 ; "282"]

A solution of 6-{2,8-dimethylimidazo[1,2-b]pyridazin-6-yl}-2-(pyrrolidin-3-yl)-3,4-dihydroisoquinolin-1-one (75.0 mg, 0.21 mmol, 1.00 equiv) and CH₂O (62.3 mg, 2.07 mmol, 10 equiv) in methanol (2 mL) was stirred for 30 min at room temperature. To the resulting mixture was added NaBH3CN (26.1 mg, 0.41 mmol, 2 equiv). The resulting mixture was stirred for an additional 1 h at room temperature, then filtered. The filtrate was purified by Prep-HPLC (Column: YMC-Actus Triart C18, 30*150 mm, 5 um; Mobile Phase A: Water (10 mmol/L NH₄HCO₃), Mobile Phase B: ACN; Flow rate: 60 mL/min; Gradient: 10% B to 65% B in 8 min) to afford 6-{2,8-dimethylimidazo[1,2-b]pyridazin-6-yl}-2-(1-methylpyrrolidin-3-yl)-3,4-dihydroisoquinolin-1-one (29.8 mg, 36.6%) as a solid.

Compounds 267-270, 281, and 282 were prepared according to the procedures outlined herein. outlined in this Example 53 and generalized by Scheme C. The table below provides intermediates used in these procedures and final compound characterization data.

| | | LCMS | |
|---|---|---|---|
| | | (ESI, | |
| | | m/z) | |
| Compound No. and Structure | Coupling Reagent | [M + H]⁺ | ¹H NMR δ |
| 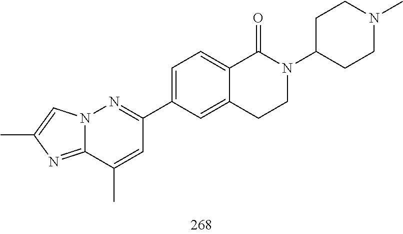 | 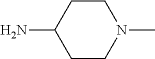 | 390 | (400 MHz, DMSO-d₆) δ 8.07 (s, 1H), 8.00 (s, 2H), 7.97 (s, 1H), 7.68 (d, J = 1.3 Hz, 1H), 4.45 (tt, J = 12.0, 4.0 Hz, 1H), 3.49 (t, J = 6.5 Hz, 2H), 3.02 (t, J = 6.5 Hz, 2H), 2.90- 2.83 (m, 2H), 2.61 (s, 3H), 2.41 (s, 3H), 2.20 (s, 3H), 2.05-1.95 (m, 2H), 1.80 (qd, J = 12.2, 3.9 Hz, 2H), 1.59-1.50 (m, 2H) |
| 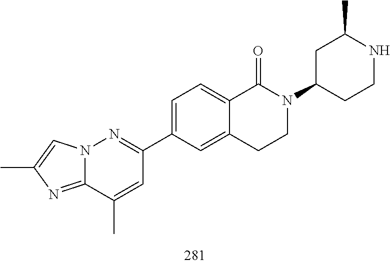 [SMILES: Cc1cn2nc(-c3ccc4c(c3)CCN([C@@H]3CCN[C@H](C)C3)C4=O)cc(C)c2n1] | 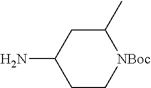 | 390 | (400 MHz, DMSO-d₆ δ 8.08 (s, 1H), 8.00 (s, 2H), 7.97 (s, 1H), 7.68 (s, 1H), 4.57 (tt, J = 12.1, 4.2 Hz, 1H), 3.48 (t, J = 6.4 Hz, 2H), 3.01 (q, J = 5.2, 4.3 Hz, 3H), 2.69-2.58 (m, 5H), 2.41 (s, 3H), 2.06 (s, 1H), 1.54 (tdd, J = 16.5, 10.2, 3.7 Hz, 3H), 1.27 (q, J = 11.6 Hz, 1H), 1.03 (d, J = 6.2 Hz, 3H) |
| 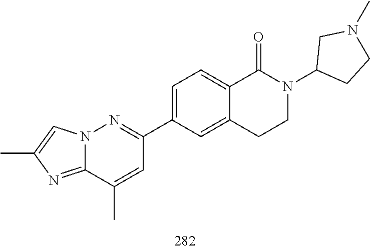 | 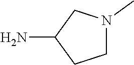 | 376 | (400 MHz, DMSO-d₆) δ 8.07 (s, 1H), 7.98 (d, J = 10.4 Hz, 3H), 7.69 (s, 1H), 5.27 (dt, J = 9.0, 4.8 Hz, 1H), 3.64 (p, J = 6.1 Hz, 2H), 3.06 (t, J = 6.5 Hz, 2H), 2.84 (t, J = 9.2 Hz, 1H), 2.74 (dd, J = 10.0, 3.6 Hz, 1H), 2.61 (s, 3H), 2.48 (s, 1H), 2.41 (s, 3H), 2.29 (s, 3H), 2.27- 2.09 (m, 2H), 1.78 (ddt, J = 13.7, 7.9, 3.9 Hz, 1H) |
| 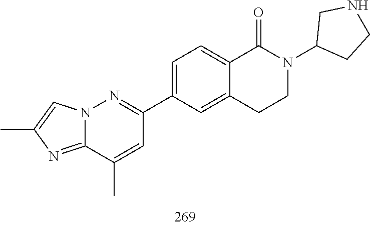 [SMILES: Cc1cn2nc(-c3ccc4c(c3)CCN(C3CCNC3)C4=O)cc(C)c2n1] | 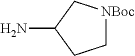 [SMILES: CC(C)(C)OC(=O)N1CCC(N)C1] | 362 | (400 MHz, DMSO-d₆) δ 8.05-7.96 (m, 3H), 7.94 (d, J = 1.7 Hz, 1H), 7.61 (q, J = 1.1 Hz, 1H), 5.10 (ddd, J = 14.5, 8.5, 5.8 Hz, 1H), 3.64-3.55 (m, 2H), 3.10-2.94 (m, 4H), 2.81 (td, J = 11.7, 11.2, 6.4 Hz, 2H), 2.63 (d, J = 1.1 Hz, 3H), 2.44 (d, J = 0.8 Hz, 3H), 2.06-1.93 (m, 1H), 1.75 (dq, J = 13.6, 7.1 Hz, 1H) |
| 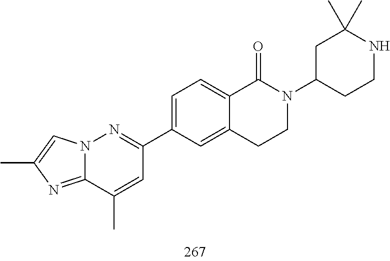 [SMILES: Cc1cn2nc(-c3ccc4c(c3)CCN(C3CCNC(C)(C)C3)C4=O)cc(C)c2n1] | 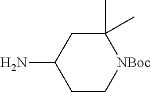 [SMILES: CC(C)(C)OC(=O)N1CCC(N)CC1(C)C] | 404 | (400 MHz, DMSO-d₆) δ 8.05 (s, 1H), 7.99 (d, J = 1.1 Hz, 2H), 7.95 (s, 1H), 7.65 (d, J = 1.3 Hz, 1H), 4.92-4.79 (m, 1H), 3.46 (t, J = 6.6 Hz, 2H), 3.03 (dd, J = 8.0, 4.3 Hz, 4H), 2.60 (d, J = 1.0 Hz, 3H), 2.40 (s, 3H), 1.77-1.59 (m, 3H), 1.59-1.50 (m, 1H), 1.24 (d, J = 16.7 Hz, 6H) |
| 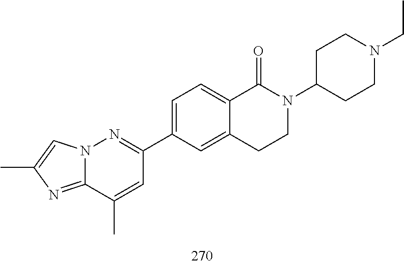 [SMILES: CCN1CCC(N2CCc3cc(-c4cc(C)c5nc(C)cn5n4)ccc3C2=O)CC1] | 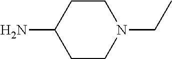 | 404 | (400 MHz, DMSO-d₆) δ 8.08 (s, 1H), 8.03-7.96 (m, 3H), 7.69 (d, J = 1.3 Hz, 1H), 4.50 (s, 1H), 3.50 (t, J = 6.4 Hz, 2H), 3.03 (t, J = 6.5 Hz, 4H), 2.64-2.59 (m, 3H), 2.41 (s, 5H), 2.09 (m,2H), 1.81 (d, J = 12.5 Hz, 2H), 1.60 (d, J = 12.1 Hz, 2H), 1.04 (t, J = 7.2 Hz, 3H) |

### Example 54

### Synthesis of Compound 252

### Synthesis of Intermediate B112

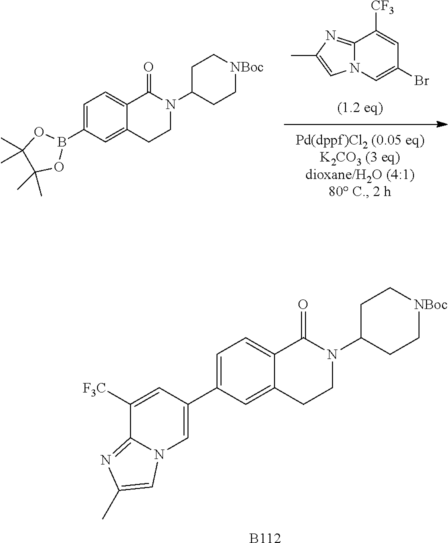 [SMILES: CC(C)(C)OC(=O)N1CCC(N2CCc3cc(B4OC(C)(C)C(C)(C)O4)ccc3C2=O)CC1 ; Cc1cn2cc(Br)cc(C(F)(F)F)c2n1 ; "B112" ; Cc1cn2cc(-c3ccc4c(c3)CCN(C3CCN(C(=O)OC(C)(C)C)CC3)C4=O)cc(C(F)(F)F)c2n1]

To a mixture of tert-butyl 4-[1-oxo-6-(4,4,5,5-tetramethyl-1,3,2-dioxaborolan-2-yl)-3,4-dihydroisoquinolin-2-yl]piperidine-1-carboxylate (90.00 mg, 0.197 mmol, 1.00 equiv) and 6-bromo-2-methyl-8-(trifluoromethyl)imidazo[1,2-a]pyridine (66.04 mg, 0.236 mmol, 1.20 equiv) in 1,4-dioxane (4 mL) and water (1 mL) was added Pd(dppf)Cl₂ (7.21 mg, 0.010 mmol, 0.05 equiv) and K₂CO₃ (81.76 mg, 0.591 mmol, 3.00 equiv) in portions at 100° C. under nitrogen atmosphere. The resulting mixture was stirred overnight at 80° C. under nitrogen atmosphere. The reaction mixture was quenched with water (10 mL) at room temperature, then extracted with ethyl acetate (3×10 mL), dried over anhydrous Na₂SO₄, and filtered. After filtration, the filtrate was concentrated under reduced pressure to afford tert-butyl 4-[6-[2-methyl-8-(trifluoromethyl) imidazo[1,2-a]pyridin-6-yl]-1-oxo-3,4-dihydroisoquinolin-2-yl]piperidine-1-carboxylate (50 mg, 47.97%) as a solid. LCMS (ES, m/z): 529 [M+H]⁺.

### Synthesis of Compound 252

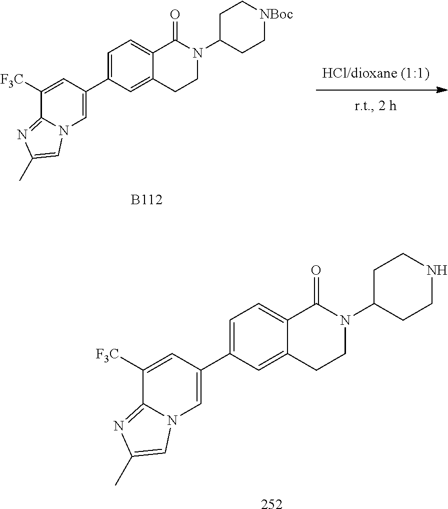 [SMILES: "B112" ; Cc1cn2cc(-c3ccc4c(c3)CCN(C3CCN(C(=O)OC(C)(C)C)CC3)C4=O)cc(C(F)(F)F)c2n1 ; Cc1cn2cc(-c3ccc4c(c3)CCN(C3CCNCC3)C4=O)cc(C(F)(F)F)c2n1 ; "252"]

To tert-butyl 4-[6-[2-methyl-8-(trifluoromethyl)imidazo[1,2-a]pyridin-6-yl]-1-oxo-3,4-dihydroisoquinolin-2-yl]piperidine-1-carboxylate (50.00 mg, 0.095 mmol, 1.00 equiv) in 1,4-dioxane (5 mL) was added HCl (gas) in 1,4-dioxane (5.00 mL) at room temperature under nitrogen atmosphere. The resulting mixture was concentrated under reduced pressure to give a residue. The residue was purified by Prep-HPLC (Column: YMC-Actus Triart C18, 30×150 mm; Mobile Phase A: water (10 mmol/L NH₄HCO₃), Mobile Phase B: ACN; Flow rate: 60 mL/min; Gradient: 15% B to 65% B in 8 min) to afford 6-[2-methyl-8-(trifluoromethyl)imidazo[1,2-a]pyridin-6-yl]-2-(piperidin-4-yl)-3,4-dihydroisoquinolin-1-one (7.1 mg, 17.52%) as a solid. LCMS (ES, m/z): 520 [M+H]⁺. ¹H NMR (400 MHz, DMSO-d6) δ 9.23 (d, J=1.8 Hz, 1H), 8.01-7.95 (m, 2H), 7.90 (s, 1H), 7.80-7.73 (m, 2H), 4.71-4.60 (m, 1H), 3.48 (t, J=6.5 Hz, 2H), 3.21 (d, J=12.5 Hz, 2H), 3.03 (t, J=6.4 Hz, 2H), 2.83 (t, J=11.7 Hz, 2H), 2.42 (s, 3H), 1.88-1.75 (m, 2H), 1.70-1.61 (m, 2H).

### Example 55

### Synthesis of Compounds 264-266, 271-275, and 277-280

### Synthesis of Intermediate B113

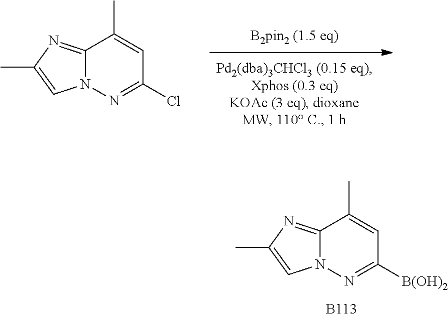

A mixture of 6-chloro-2,8-dimethylimidazo[1,2-b]pyridazine (4.50 g, 24.77 mmol, 1.00 equiv), B₂pin₂ (6.92 g, 27.25 mmol, 1.1 equiv), KOAc (7.30 g, 74.33 mmol, 3 equiv), Xphos (1.18 g, 2.47 mmol, 0.1 equiv) and Pd₂(dba)₃CHCl₃ (1.28 g, 1.24 mmol, 0.05 equiv) in dioxane (135 mL) was irradiated with microwave radiation for 1 h at 110° C. The resulting mixture was filtered to afford intermediate B113. LCMS (ES, m/z): 192 [M+H]⁺.

### Synthesis of Intermediate B114

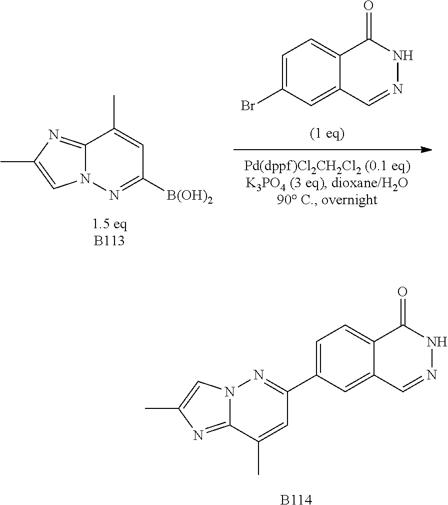

A mixture of 2,8-dimethylimidazo[1,2-b]pyridazin-6-ylboronic acid (3.44 g, 17.99 mmol, 1.5 equiv), 6-bromo-2H-phthalazin-1-one (2.7 g, 11.998 mmol, 1.00 equiv), K₃PO₄ (7.64 g, 35.99 mmol, 3 equiv), and Pd(dppf)Cl₂CH₂Cl₂ (0.98 g, 1.20 mmol, 0.1 equiv) in dioxane (150 mL) and water (30 mL) was stirred overnight at 90° C. under nitrogen atmosphere. The reaction mixture was quenched with water (100 mL) at room temperature. The resulting mixture was filtered, the filter cake was washed with ethyl acetate (3×30 mL). The filtrate was concentrated under reduced pressure to afford 6-{2,8-dimethylimidazo[1,2-b]pyridazin-6-yl}-2H-phthalazin-1-one (3.3 g, 94.4%) as a solid. LCMS (ES, m/z): 292 [M+H]⁺.

### Synthesis of Intermediate B115

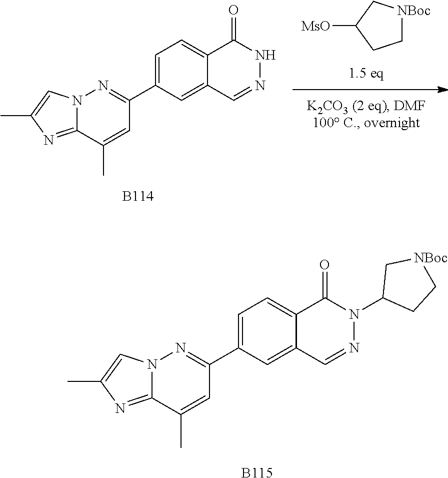 [SMILES: "B114" ; "B115" ; Cc1cn2nc(-c3ccc4c(=O)[nH]ncc4c3)cc(C)c2n1 ; Cc1cn2nc(-c3ccc4c(=O)n(C5CCN(C(=O)OC(C)(C)C)C5)ncc4c3)cc(C)c2n1 ; CC(C)(C)OC(=O)N1CCC(OS(C)(=O)=O)C1]

A mixture of 6-{2,8-dimethylimidazo[1,2-b]pyridazin-6-yl}-2H-phthalazin-1-one (220.0 mg, 0.75 mmol, 1.00 equiv), tert-butyl 3-(methanesulfonyloxy)pyrrolidine-1-carboxylate (300.5 mg, 1.13 mmol, 1.5 equiv), and K₂CO₃ (313.1 mg, 2.26 mmol, 3 equiv) in DMF (4 mL) was stirred overnight at 100° C. The resulting mixture was diluted with water (10 mL), then extracted with ethyl acetate (3×10 mL). The combined organic layers were washed with brine (1×30 mL), dried over anhydrous Na₂SO₄, and filtered. After filtration, the filtrate was concentrated under reduced pressure to afford tert-butyl 3-(6-{2,8-dimethylimidazo[1,2-b]pyridazin-6-yl}-1-oxophthalazin-2-yl)pyrrolidine-1-carboxylate (270.0 mg, 77.6%) as an oil. LCMS (ES, m/z): 461 [M+H]⁺.

### Synthesis of Compound 264

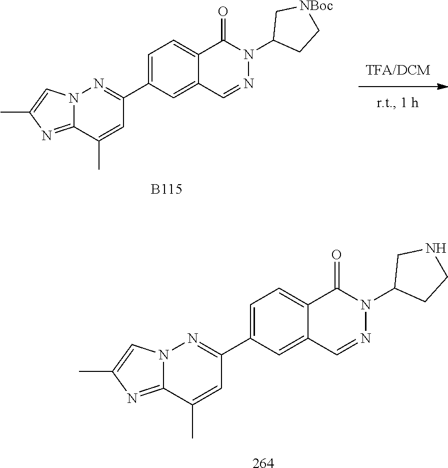 [SMILES: "B115" ; Cc1cn2nc(-c3ccc4c(=O)n(C5CCN(C(=O)OC(C)(C)C)C5)ncc4c3)cc(C)c2n1 ; Cc1cn2nc(-c3ccc4c(=O)n(C5CCNC5)ncc4c3)cc(C)c2n1 ; "264"]

A solution of tert-butyl 3-(6-{2,8-dimethylimidazo[1,2-b]pyridazin-6-yl}-1-oxophthalazin-2-yl)pyrrolidine-1-carboxylate (270.0 mg, 0.58 mmol, 1.00 equiv) in DCM (3 mL) and TFA (0.75 mL) was stirred for 1 h at room temperature. The resulting mixture was concentrated under vacuum to give a residue. The residue was purified by Prep-HPLC (Column: YMC-Actus Triart C18, 30*150 mm, Sum; Mobile Phase A: Water (10 mmol/L NH₄HCO₃), Mobile Phase B: ACN; Flow rate: 60 mL/min; Gradient: 5% B to 45% B in 8 min) to afford 6-{2,8-dimethylimidazo[1,2-b]pyridazin-6-yl}-2-(pyrrolidin-3-yl)phthalazin-1-one (23 mg, 10.8%) as a solid. LCMS (ES, m/z): 361 [M+H]⁺.

### Synthesis of Compound 271

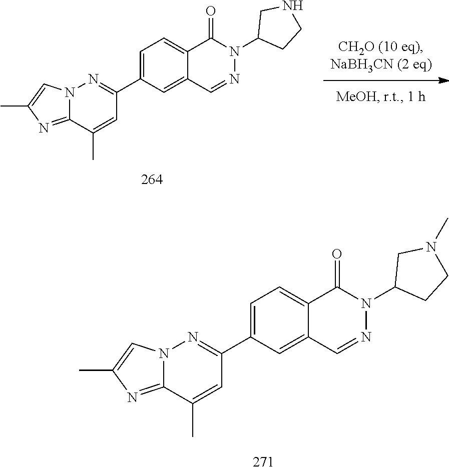

A mixture of 6-{2,8-dimethylimidazo[1,2-b]pyridazin-6-yl}-2-(pyrrolidin-3-yl)phthalazin-1-one (200.0 mg, 0.55 mmol, 1.00 equiv) and HCHO (166.4 mg, 5.55 mmol, 10 equiv) in methanol (5 mL) was stirred for 30 min at room temperature. To the reaction mixture was added NaBH3CN (69.7 mg, 1.11 mmol, 2 equiv). The resulting mixture was stirred for an additional 1 h at room temperature. The resulting mixture was filtered, the filtrate was purified by Prep-HPLC (Column: YMC-Actus Triart C18, 30*150 mm, Sum; Mobile Phase A: Water(10 mmol/L NH₄HCO₃), Mobile Phase B: ACN; Flow rate: 60 mL/min; Gradient: 45% B to 67% B in 8 min) to afford 6-{2,8-dimethylimidazo[1,2-b]pyridazin-6-yl}-2-(1-methylpyrrolidin-3-yl)phthalazin-1-one (10.9 mg, 5.2%) as a solid. LCMS (ES, m/z): 375 [M+H]⁺.

Compounds 264-266, 271-275, 277-280 were prepared according to the procedures outlined herein. outlined in this Example 55 and generalized by Scheme D. The table below provides intermediates used in these procedures and final compound characterization data.

| | | LCMS | |
|---|---|---|---|
| | | (ESI, | |
| | | m/z) | |
| Compound No. and Structure | Coupling Reagent | [M + H]⁺ | ¹H NMR δ |
| 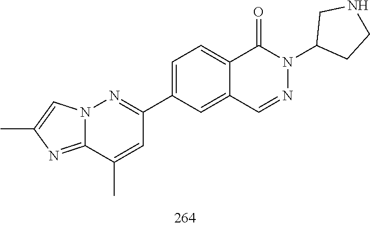 [SMILES: Cc1cn2nc(-c3ccc4c(=O)n(C5CCNC5)ncc4c3)cc(C)c2n1] | 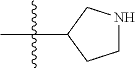 [SMILES: *C1CCNC1] | 361 | (400 MHz, DMSO-d₆) δ 8.55 (d, J = 7.0 Hz, 2H), 8.45 (dd, J = 8.5, 1.8 Hz, 1H), 8.36 (d, J = 8.5 Hz, 1H), 8.07 (s, 1H), 7.73 (d, J = 1.3 Hz, 1H), 5.49 (dt, J = 12.2, 4.8 Hz, 1H), 3.17 (dd, J = 11.9, 7.4 Hz, 1H), 3.05 (dt, J = 11.3, 7.4 Hz, 1H), 2.91 (ddd, J = 18.2, 11.3, 6.5 Hz, 2H), 2.61 (s, 3H), 2.40 (s, 3H), 2.21- 1.97 (m, 2H) |
| 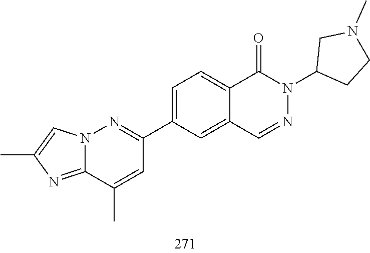 [SMILES: Cc1cn2nc(-c3ccc4c(=O)n(C5CCN(C)C5)ncc4c3)cc(C)c2n1] | 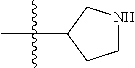 [SMILES: *C1CCNC1] | 375 | (400 MHz, DMSO-d₆) δ 8.61 (d, J = 1.8 Hz, 2H), 8.49 (dd, J = 8.5, 1.8 Hz, 1H), 8.37 (d, J = 8.4 Hz, 1H), 8.12 (d, J = 1.0 Hz, 1H), 7.80 (d, J = 1.2 Hz, 1H), 5.66-5.54 (m, 1H), 2.99 (dd, J = 9.3, 7.9 Hz, 1H), 2.74- 2.65 (m, 2H), 2.64 (d, J = 1.1 Hz, 3H), 2.59 (dd, J = 9.3, 6.3 Hz, 1H), 2.43 (d, J = 0.9 Hz, 3H), 2.30 (s, 3H), 2.25 (ddt, J = 12.8, 9.8, 4.9 Hz, 1H), 2.12 (ddt, J = 12.9, 7.7, 5.1 Hz, 1H) |
| 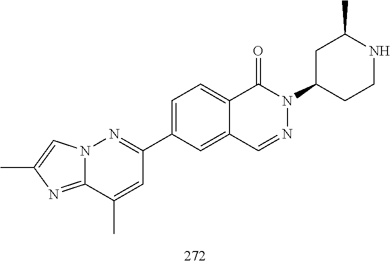 [SMILES: Cc1cn2nc(-c3ccc4c(=O)n([C@@H]5CCN[C@H](C)C5)ncc4c3)cc(C)c2n1] | 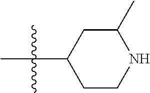 [SMILES: *C1CCNC(C)C1] | 389 | (400 MHz, DMSO-d₆) δ 8.52 (s, 1H), 8.47 (s, 1H), 8.39 (d, J = 8.5 Hz, 1H), 8.30 (d, J = 8.5 Hz, 1H), 8.01 (s, 1H), 7.66 (s, 1H), 5.05 (tt, J = 11.7, 4.5 Hz, 1H), 3.23 (d, J = 12.4 Hz, 1H), 3.05 (d, J = 10.5 Hz, 1H), 2.90 (t, J = 12.0 Hz, 1H), 2.58 (s, 3H), 2.38 (s, 3H), 1.86 (td, J = 45.6, 42.0, 11.5 Hz, 4H), 1.17 (d, J = 6.2 Hz, 3H) |
| 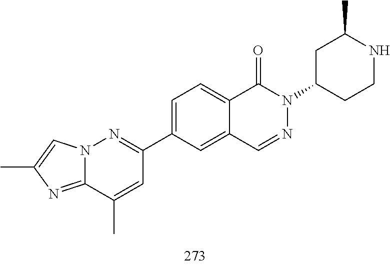 [SMILES: Cc1cn2nc(-c3ccc4c(=O)n([C@H]5CCN[C@H](C)C5)ncc4c3)cc(C)c2n1] | 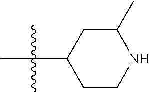 [SMILES: *C1CCNC(C)C1] | 389 | (400 MHz, DMSO-d₆) δ 8.49 (d, J = 2.2 Hz, 2H), 8.41 (dd, J = 8.4, 1.8 Hz, 1H), 8.36 (d, J = 8.4 Hz, 1H), 8.00 (d, J = 1.0 Hz, 1H), 7.65 (d, J = 1.3 Hz, 1H), 5.21 (tt, J = 8.9, 4.5 Hz, 1H), 3.33 (dt, J = 6.9, 4.8 Hz, 1H), 3.00-2.85 (m, 2H), 2.63 (d, J = 1.1 Hz, 3H), 2.42 (d, J = 0.8 Hz, 3H), 2.05 (ddd, J = 13.3, 9.1, 4.6 Hz, 1H), 1.97-1.84 (m, 1H), 1.77 (dq, J = 13.3, 4.9 Hz, 1H), 1.59 (dtd, J = 13.0, 4.8, 1.4 Hz, 1H), 1.15 (d, J = 6.8 Hz, 3H) |
| 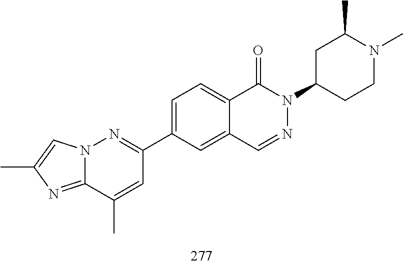 | 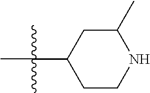 [SMILES: *C1CCNC(C)C1] | 403 | (400 MHz, DMSO-d₆) δ 8.65- 8.58 (m, 2H), 8.51 (dd, J = 8.4, 1.8 Hz, 1H), 8.40 (d, J = 8.4 Hz, 1H), 8.14 (s, 1H), 7.81 (d, J = 1.3 Hz, 1H), 5.01-4.87 (m, 1H), 2.93 (dt, J = 11.4, 3.3 Hz, 1H), 2.67-2.63 (m, 3H), 2.44 (s, 3H), 2.21 (s, 4H), 2.12-1.96 (m, 2H), 1.75 (q, J = 9.0, 6.9 Hz, 3H), 1.08 (d, J = 6.1 Hz, 3H) |
| 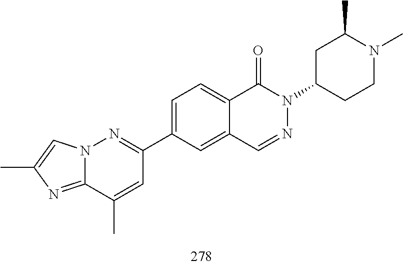 | 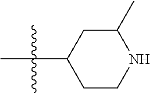 [SMILES: *C1CCNC(C)C1] | 403 | (400 MHz, DMSO-d₆) δ 8.64- 8.55 (m, 2H), 8.50 (dd, J = 8.4, 1.8 Hz, 1H), 8.39 (d, J = 8.4 Hz, 1H), 8.14 (s, 1H), 7.81 (d, J = 1.3 Hz, 1H), 5.23-5.13 (m, 1H), 3.13 (s, 1H), 2.65 (s, 5H), 2.44 (s, 3H), 2.30 (s, 3H), 2.19 (td, J = 11.9, 4.7 Hz, 1H), 2.06 (s, 1H), 1.77 (s, 1H), 1.65 (d, J = 12.4 Hz, 1H), 1.08 (d, J = 6.6 Hz, 3H) |
| 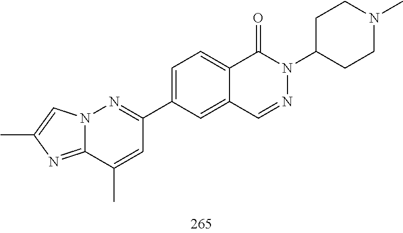 | 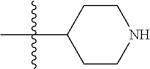 [SMILES: *C1CCNCC1] | 389 | (400 MHz, DMSO-d₆) δ 8.63- 8.55 (m, 2H), 8.49 (dd, J = 8.4, 1.8 Hz, 1H), 8.39 (d, J = 8.4 Hz, 1H), 8.13 (s, 1H), 7.80 (d, J = 1.3 Hz, 1H), 4.87 (dd, J = 11.2, 6.9 Hz, 1H), 2.92 (d, J = 7.7 Hz, 2H), 2.64 (d, J = 1.0 Hz, 3H), 2.43 (s, 3H), 2.23 (s, 3H), 2.13-1.96 (m, 4H), 1.82-1.71 (m, 2H) |
| 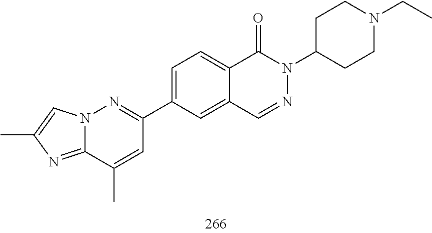 [SMILES: CCN1CCC(n2ncc3cc(-c4cc(C)c5nc(C)cn5n4)ccc3c2=O)CC1] | 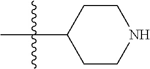 [SMILES: *C1CCNCC1] | 403 | (400 MHz, DMSO-d₆) δ 8.64- 8.57 (m, 2H), 8.50 (dd, J = 8.4, 1.7 Hz, 1H), 8.40 (d, J = 8.4 Hz, 1H), 8.14 (s, 1H), 7.80 (d, J = 1.2 Hz, 1H), 4.94-4.84 (m, 1H), 3.03 (d, J = 8.6 Hz, 2H), 2.65 (s, 3H), 2.43 (s, 3H), 2.39 (q, J = 7.2, 6.5 Hz, 2H), 2.07-1.94 (m, 4H), 1.78 (d, J = 10.8 Hz, 2H), 1.04 (t, J = 7.2 Hz, 3H) |
| 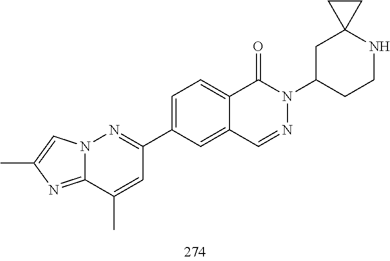 [SMILES: Cc1cn2nc(-c3ccc4c(=O)n(C5CCNC6(CC6)C5)ncc4c3)cc(C)c2n1] | 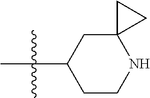 [SMILES: *C1CCNC2(CC2)C1] | 401 | (400 MHz, DMSO-d₆) δ 8.60- 8.55 (m, 2H), 8.47 (dd, J = 8.5, 1.7 Hz, 1H), 8.37 (d, J = 8.4 Hz, 1H), 8.10 (s, 1H), 7.75 (d, J = 1.3 Hz, 1H), 5.15 (tt, J = 11.7, 4.1 Hz, 1H), 3.12-3.03 (m, 1H), 2.79 (td, J = 12.9, 3.0 Hz, 1H), 2.63 (s, 3H), 2.42 (s, 3H), 2.36 (t, J = 11.6 Hz, 1H), 1.92 (qd, J = 12.3, 4.3 Hz, 1H), 1.81 (d, J = 12.3 Hz, 1H), 1.20 (dq, J = 12.5, 1.7 Hz, 1H), 0.65 (ddd, J = 9.6, 5.7, 3.6 Hz, 1H), 0.60- 0.53 (m, 1H), 0.50 (dt, J = 9.4, 4.0 Hz, 1H), 0.42 (ddd, J = 9.0, 5.8, 3.2 Hz, 1H) |
| 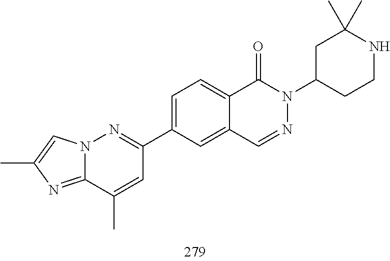 [SMILES: Cc1cn2nc(-c3ccc4c(=O)n(C5CCNC(C)(C)C5)ncc4c3)cc(C)c2n1] | 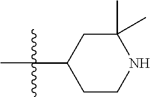 [SMILES: *C1CCNC(C)(C)C1] | 403 | (400 MHz, DMSO-d₆) δ 8.67- 8.59 (m, 2H), 8.52 (dd, J = 8.5, 1.8 Hz, 1H), 8.40 (d, J = 8.4 Hz, 1H), 8.14 (s, 1H), 7.81 (s, 1H), 5.36- 5.25 (m, 1H), 3.15-3.10 (m, 2H), 2.65 (s, 3H), 2.44 (s, 3H), 1.97 (s, 1H), 1.85 (s, 2H), 1.76 (d, J = 12.7 Hz, 1H), 1.32 (s, 3H), 1.25 (s, 3H) |
| 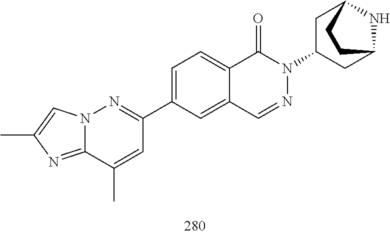 | 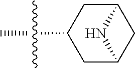 | 401 | (400 MHz, DMSO-d₆) δ 8.62-8.57 (m, 2H), 8.49 (dd, J = 8.4, 1.7 Hz, 1H), 8.38 (d, J = 8.4 Hz, 1H), 8.13 (s, 1H), 7.80 (s, 1H), 5.27 (p, J = 8.6 Hz, 1H), 3.55 (d, J = 8.3 Hz, 2H), 2.64 (s, 3H), 2.43 (s, 3H), 2.26 (dt, J = 13.3, 8.2 Hz, 2H), 1.75-1.59 (m, 6H) |
| 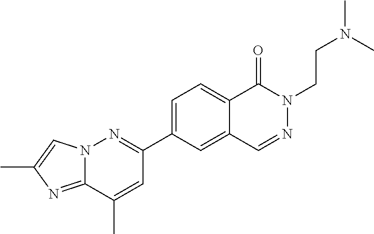 | 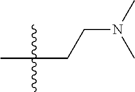 [SMILES: *CCN(C)C] | 363 | (400 MHz, DMSO-d₆) δ 10.05 (s, 1H), 8.75 (d, J = 1.7 Hz, 1H), 8.64 (s, 1H), 8.57 (dd, J = 8.4, 1.8 Hz, 1H), 8.49-8.40 (m, 2H), 8.24 (s, 1H), 4.55 (t, J = 6.0 Hz, 2H), 3.56 (q, J = 5.8 Hz, 2H), 2.88 (d, J = 4.7 Hz, 6H), 2.75 (s, 3H), 2.55 (s, 3H) |
| 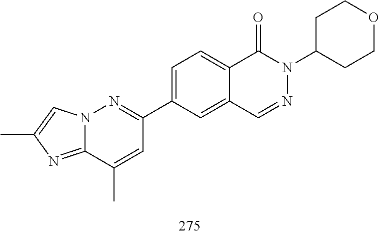 [SMILES: Cc1cn2nc(-c3ccc4c(=O)n(C5CCOCC5)ncc4c3)cc(C)c2n1] | 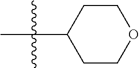 [SMILES: *C1CCOCC1] | 376 | (400 MHz, DMSO-d₆) δ 8.66- 8.60 (m, 2H), 8.52 (dd, J = 8.5, 1.8 Hz, 1H), 8.42 (d, J = 8.4 Hz, 1H), 8.14 (s, 1H), 7.81 (d, J = 1.2 Hz, 1H), 5.16 (tt, J = 11.6, 4.1 Hz, 1H), 4.01 (dd, J = 11.0, 4.4 Hz, 2H), 3.54 (dd, J = 12.6, 10.6 Hz, 2H), 2.66 (d, J = 1.0 Hz, 3H), 2.44 (s, 2H), 1.81-1.72 (m, 2H), 1.24 (s, 1H) |
| 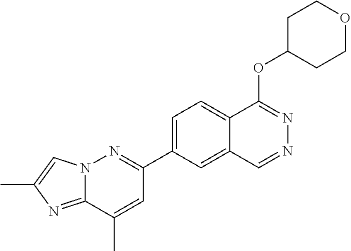 [SMILES: Cc1cn2nc(-c3ccc4c(OC5CCOCC5)nncc4c3)cc(C)c2n1] | 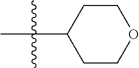 [SMILES: *C1CCOCC1] | 376 | (400 MHz, DMSO-d₆) δ 9.43 (d, J = 0.8 Hz, 1H), 8.80 (d, J = 1.7 Hz, 1H), 8.64 (dd, J = 8.6, 1.8 Hz, 1H), 8.34 (d, J = 8.6 Hz, 1H), 8.15 (d, J = 0.9 Hz, 1H), 7.84 (d, J = 1.2 Hz, 1H), 5.64 (tt, J = 8.4, 4.0 Hz, 1H), 3.97 (dt, J = 11.5, 4.5 Hz, 2H), 3.62 (ddd, J = 11.7, 8.9, 3.0 Hz, 2H), 2.66 (d, J = 1.0 Hz, 3H), 2.44 (d, J = 0.8 Hz, 3H), 2.24-2.14 (m, 2H), 1.85 (dtd, J = 12.8, 8.7, 3.9 Hz, 2H) |
| 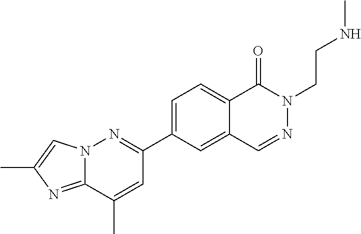 | 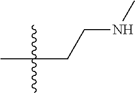 [SMILES: *CCNC] | 349 | (400 MHz, DMSO-d₆) δ 8.51 (s, 1H), 8.46 (s, 1H), 8.44-8.37 (m, 1H), 8.31 (d, J = 8.4 Hz, 1H), 8.05 (s, 1H), 7.70 (s, 1H), 4.21 (t, J = 6.5 Hz, 2H), 2.88 (t, J = 6.5 Hz, 2H), 2.60 (s, 3H), 2.40 (s, 3H), 2.31 (s, 3H) |

### Example 56

### Synthesis of Compounds 248-251 and 253-263

### Synthesis of Intermediate B116

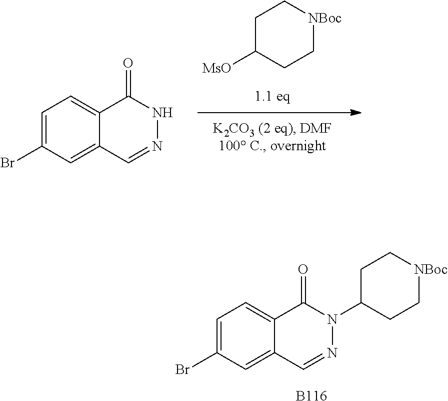

A mixture of 6-bromo-2H-phthalazin-1-one (2.00 g, 8.88 mmol, 1.00 equiv),tert-butyl 4-(methanesulfonyloxy)piperidine-1-carboxylate (2.73 g, 9.77 mmol, 1.10 equiv), and K₂CO₃ (2.46 g, 17.77 mmol, 2.00 equiv) in DMF (40.00 mL) was stirred overnight at 100° C. The resulting mixture was diluted with water (100 mL), then extracted with ethyl acetate (3×100 mL). The combined organic layers were washed with brine (1×100 mL), dried over anhydrous Na₂SO₄, and filtered. After filtration, the filtrate was concentrated under reduced pressure to give a residue. The residue was purified by silica gel column chromatography, eluted with PE/EA (72/28) to afford tert-butyl 4-(6-bromo-1-oxophthalazin-2-yl)piperidine-1-carboxylate (2.90 g, 79.92%) as a solid. LCMS (ES, m/z): 408 [M+H]⁺.

### Synthesis of Intermediate B117

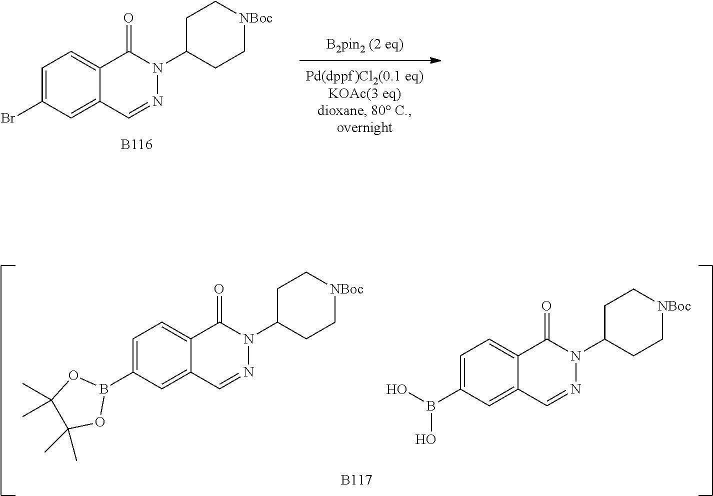 [SMILES: "B116" ; CC(C)(C)OC(=O)N1CCC(n2ncc3cc(Br)ccc3c2=O)CC1 ; CC(C)(C)OC(=O)N1CCC(n2ncc3cc(B4OC(C)(C)C(C)(C)O4)ccc3c2=O)CC1 ; "B117" ; CC(C)(C)OC(=O)N1CCC(n2ncc3cc(B(O)O)ccc3c2=O)CC1]

A mixture of tert-butyl 4-(6-bromo-1-oxophthalazin-2-yl)piperidine-1-carboxylate (2.60 g, 6.36 mmol, 1.00 equiv), B₂PIN₂ (3.23 g, 12.73 mmol, 2 equiv), Pd(dppf)Cl₂ CH₂Cl₂ (0.52 g, 0.63 mmol, 0.1 equiv), and KOAc (1.87 g, 19.104 mmol, 3 equiv) in dioxane (52.00 mL) was stirred overnight at 80° C. under N2 atmosphere. The resulting mixture was filtered. LCMS (ES, m/z): 456/374 [M+H]⁺.

### Synthesis of Intermediate B118

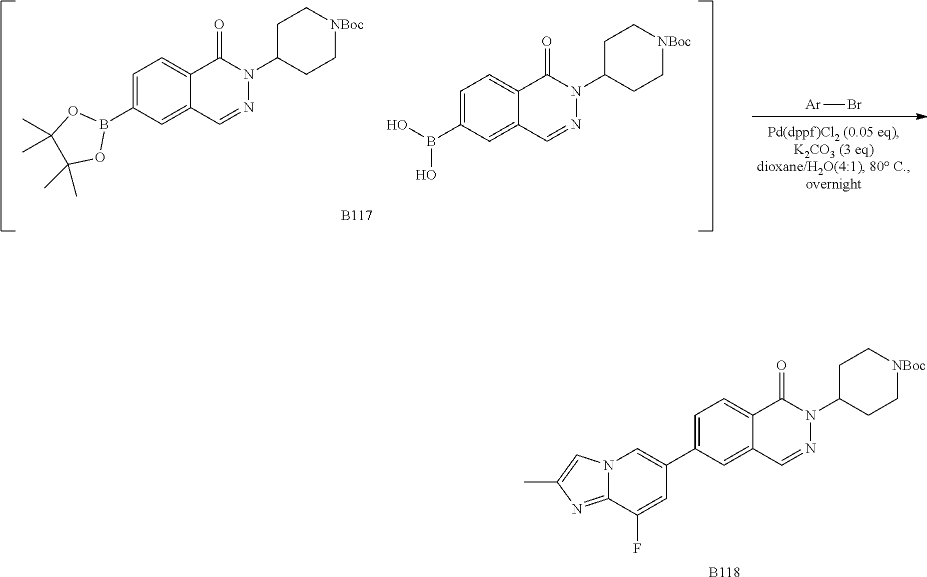

A mixture of tert-butyl 4-[1-oxo-6-(4,4,5,5-tetramethyl-1,3,2-dioxaborolan-2-yl)phthalazin-2-yl]piperidine-1-carboxylate (125.0 mg, 0.27 mmol, 1.00 equiv), 6-bromo-8-fluoro-2-methylimidazo[1,2-a]pyridine (94.3 mg, 0.41 mmol, 1.50 equiv), Pd(dppf)Cl₂ CH₂Cl₂ (11.2 mg, 0.01 mmol, 0.05 equiv), and K₂CO₃ (113.8 mg, 0.83 mmol, 3.00 equiv) in dioxane (2.00 mL) and water (0.50 mL) was stirred for 12 h at 80° C. under nitrogen atmosphere. The resulting mixture was diluted with water (20 mL), then extracted with ethyl acetate (3×20 mL). The combined organic layers were washed with brine (1×20 mL), dried over anhydrous Na₂SO₄, and filtered. After filtration, the filtrate was concentrated under reduced pressure to give a residue. The residue was purified by silica gel column chromatography, eluted with CH₂Cl_{2/}MeOH (97/3) to afford tert-butyl 4-(6-[8-fluoro-2-methylimidazo[1,2-a]pyridin-6-yl]-1-oxophthalazin-2-yl)piperidine-1-carboxylate (110.0 mg, 83.9%) as an oil. LCMS (ES, m/z): 478 [M+H]⁺.

### Synthesis of Compound 248

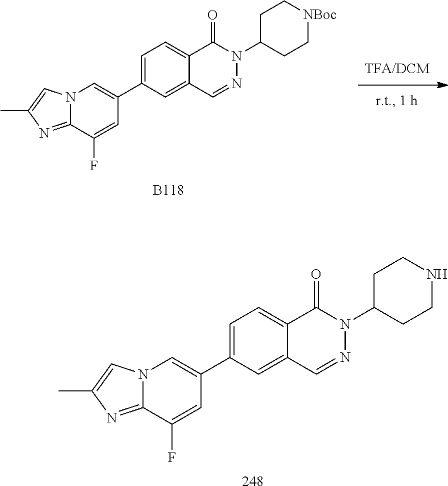 [SMILES: "B118" ; Cc1cn2cc(-c3ccc4c(=O)n(C5CCN(C(=O)OC(C)(C)C)CC5)ncc4c3)cc(F)c2n1 ; Cc1cn2cc(-c3ccc4c(=O)n(C5CCNCC5)ncc4c3)cc(F)c2n1 ; "248"]

A solution of tert-butyl 4-(6-[8-fluoro-2-methylimidazo[1,2-a]pyridin-6-yl]-1-oxophthalazin-2-yl)piperidine-1-carboxylate (110.0 mg, 0.23 mmol, 1.00 equiv) in DCM (2.00 mL) and TFA (0.50 mL) was stirred for 1 h at room temperature. The resulting mixture was concentrated under vacuum. The crude product (100 mg) was purified by Prep-HPLC with the following conditions (Column: YMC-Actus Triart C18, 30*150 mm, 5|im; Mobile Phase A: Water (10 mmol/L NH₄HCO₃), Mobile Phase B: ACN; Flow rate: 60 mL/min; Gradient: 5% B to 50% B in 8 min, 50% B; Wave Length: 220 nm) to afford 6-[8-fluoro-2-methylimidazo[1,2-a]pyridin-6-yl]-2-(piperidin-4-yl)phthalazin-1-one(27.1 mg, 30.74%) as a solid.

Compounds 248-251 and 253-263 were prepared according to the procedures outlined herein. outlined in this Example 56 and generalized by Scheme E. The table below provides intermediates used in these procedures and final compound characterization data.

| | | LCMS | |
|---|---|---|---|
| | | (ESI, m/z) | |
| Compound No. and Structure | Coupling Reagent | [M + H]⁺ | ¹H NMR δ |
| 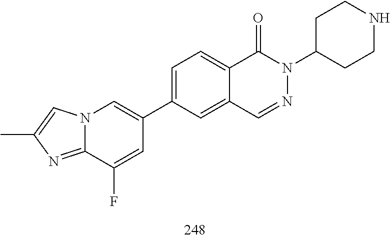 [SMILES: Cc1cn2cc(-c3ccc4c(=O)n(C5CCNCC5)ncc4c3)cc(F)c2n1] | 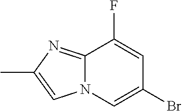 | 378 | (400 MHz, DMSO-d₆) δ 9.00 (d, J = 1.6 Hz, 1H), 8.49 (s, 1H), 8.38-8.29 (m, 2H), 8.22 (dd, J = 8.4, 2.0 Hz, 1H), 7.89 (d, J = 3.0 Hz, 1H), 7.68 (dd, J = 12.5, 1.6 Hz, 1H), 4.97 (tt, J = 11.7, 4.1 Hz, 1H), 3.09 (d, J = 12.5 Hz, 2H), 2.69-2.59 (m, 2H), 2.40 (s, 3H), 1.88 (qd, J = 12.1, 4.1 Hz, 2H), 1.72 (dd, J = 12.4, 3.7 Hz, 2H). ¹⁹F NMR (376 MHz, DMSO) δ −73.40, −131.85 |
| 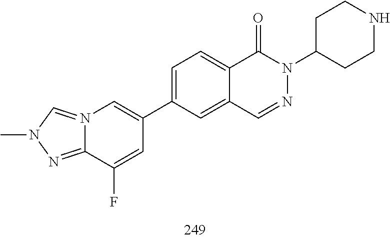 | 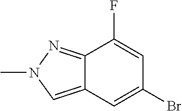 | 378 | (400 MHz, DMSO-d₆) δ 8.62 (s, 1H), 8.52 (s, 1H), 8.36 (d, J = 1.8 Hz, 1H), 8.34-8.26 (m, 2H), 7.96 (d, J = 1.2 Hz, 1H), 7.35 (dd, J = 12.0, 1.2 Hz, 1H), 4.97 (tt, J = 11.7, 4.1 Hz, 1H), 4.24 (s, 3H), 3.12-3.04 (m, 2H), 2.63 (td, J = 12.6, 2.5 Hz, 2H), 1.88 (qd, J = 12.1, 4.1 Hz, 2H), 1.75-1.67 (m, 2H); ¹⁹F NMR (376 MHz, DMSO) δ −116.30, −116.72, −117.41 |
| 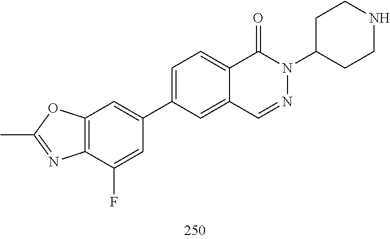 [SMILES: Cc1nc2c(F)cc(-c3ccc4c(=O)n(C5CCNCC5)ncc4c3)cc2o1] | 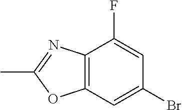 | 379 | (400 MHz, DMSO-d₆) δ 8.51 (s, 1H), 8.39 (d, 1H), 8.27 (m, J = 8.4, 1.9 Hz, 2H), 8.10 (d, J = 1.4 Hz, 1H), 7.77 (dd, J = 11.4, 1.5 Hz, 1H), 4.96 (tt, J = 11.7, 4.0 Hz, 1H), 3.12-3.04 (m, 2H), 2.70 (s, 3H), 2.63 (td, J = 12.5, 2.6 Hz, 2H), 1.87 (qd, J = 12.1, 4.1 Hz, 2H), 1.75- 1.66 (m, 2H); ¹⁹F NMR (376 MHz, DMSO) δ −73.40, −125.74 |
| 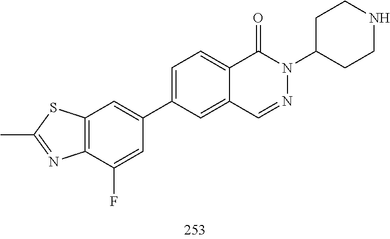 [SMILES: Cc1nc2c(F)cc(-c3ccc4c(=O)n(C5CCNCC5)ncc4c3)cc2s1] | 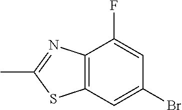 | 395 | (400 MHz, DMSO-d₆) δ 8.51 (s, 1H), 8.44 (d, J = 1.6 Hz, 1H), 8.40-8.31 (m, 2H), 8.27 (dd, J = 8.4, 1.8 Hz, 1H), 7.87 (dd, J = 12.1, 1.7 Hz, 1H), 4.96 (tt, J = 11.6, 4.0 Hz, 1H), 3.12- 3.04 (m, 2H), 2.88 (s, 3H), 2.63 (td, J = 12.5, 2.5 Hz, 2H), 1.87 (qd, J = 12.1, 4.1 Hz, 2H), 1.75-1.66 (m, 2H); ¹⁹F NMR (376 MHz, DMSO) δ −122.53 |
| 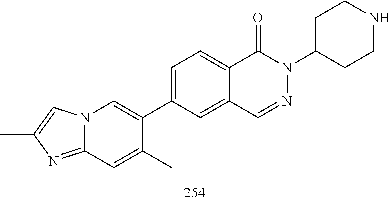 [SMILES: Cc1cn2cc(-c3ccc4c(=O)n(C5CCNCC5)ncc4c3)c(C)cc2n1] | 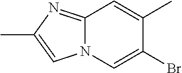 | 374 | (400 MHz, DMSO-d₆) δ 8.52 (s, 1H), 8.48 (s, 1H), 8.33 (d, J = 8.2 Hz, 1H), 8.01 (d, J = 1.7 Hz, 1H), 7.92 (dd, J = 8.3, 1.7 Hz, 1H), 7.64 (s, 1H), 7.41 (s, 1H), 4.97 (tt, J = 11.6,4.1 Hz, 1H), 3.12-3.04 (m, 2H), 2.63 (td, J = 12.4, 2.4 Hz, 2H), 2.34 (s, 3H), 2.25 (s, 3H), 1.87 (qd, J = 12.1, 4.1 Hz, 2H), 1.71 (dd, J = 12.3, 3.9 Hz, 2H) |
| 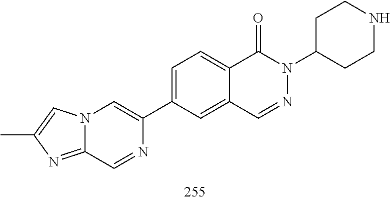 [SMILES: Cc1cn2cc(-c3ccc4c(=O)n(C5CCNCC5)ncc4c3)ncc2n1] | 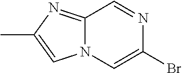 | 361 | (400 MHz, DMSO-d₆) δ 9.41 (d, J = 1.5 Hz, 1H), 9.09 (d, J = 1.4 Hz, 1H), 8.64-8.57 (m, 2H), 8.48 (dd, J = 8.5, 1.8 Hz, 1H), 8.37 (d, J = 8.5 Hz, 1H), 7.97 (s, 1H), 4.96 (tt, J = 11.6, 4.0 Hz, 1H), 3.12-3.04 (m, 2H), 2.63 (td, J = 12.6, 2.6 Hz, 2H), 2.46 (s, 3H), 1.87 (qd, J = 12.1, 4.1 Hz, 2H), 1.75-1.66 (m, 2H) |
| 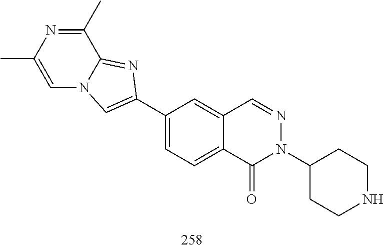 [SMILES: Cc1cn2cc(-c3ccc4c(=O)n(C5CCNCC5)ncc4c3)nc2c(C)n1] | 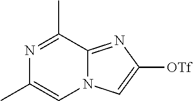 [SMILES: Cc1cn2cc(OS(=O)(=O)C(F)(F)F)nc2c(C)n1] | 375 | (400 MHz, DMSO-d₆) δ 9.07 (s, 1H), 8.90-8.78 (m, 2H), 8.63 (d, J = 11.6 Hz, 2H), 8.50 (d, J = 8.7 Hz, 2H), 8.37 (d, J = 8.4 Hz, 1H), 5.21 (td, J = 11.4, 5.4 Hz, 1H), 3.41 (s, 2H), 3.17 (q, J = 12.2 Hz, 2H), 2.87 (d, J = 7.5 Hz, 3H), 2.45 (d, J = 3.8 Hz, 3H), 2.21 (q, J = 10.1, 6.8 Hz, 2H), 1.98 (dd, J = 13.7, 3.8 Hz, 2H) |
| 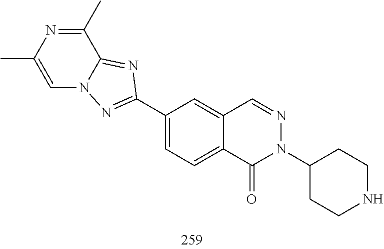 [SMILES: Cc1cn2nc(-c3ccc4c(=O)n(C5CCNCC5)ncc4c3)nc2c(C)n1] | 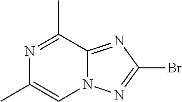 | 376 | (400 MHz, DMSO-d₆) δ 8.85 (s, 1H), 8.77 (s, 1H), 8.71 (s, 1H), 8.62 (d, J = 8.4 Hz, 1H), 8.43 (d, J = 8.4 Hz, 1H), 5.01- 4.91 (m, 1H), 3.08 (d, J = 12.1 Hz, 2H), 2.86 (s, 3H), 2.63 (t, J = 11.9 Hz, 2H), 2.53 (s, 3H), 1.87 (qd, J = 12.5, 4.1 Hz, 2H), 1.71 (d, J = 11.4 Hz, 2H) |
| 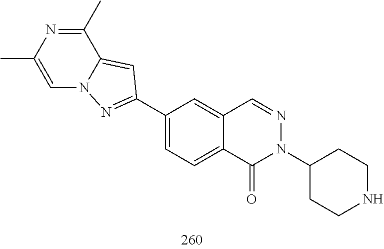 [SMILES: Cc1cn2nc(-c3ccc4c(=O)n(C5CCNCC5)ncc4c3)cc2c(C)n1] | 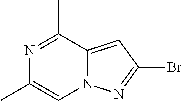 | 375 | (400 MHz, DMSO-d₆) δ 8.60- 8.52 (m, 3H), 8.47 (dd, J = 8.3, 1.7 Hz, 1H), 8.36 (d, J = 8.3 Hz, 1H), 7.67 (s, 1H), 4.96 (tt, J = 11.7, 4.1 Hz, 1H), 3.11- 3.03 (m, 2H), 2.73 (s, 3H), 2.62 (td, J = 12.5, 2.5 Hz, 2H), 2.45 (s, 3H), 1.87 (qd, J = 12.1, 4.1 Hz, 2H), 1.75-1.66 (m, 2H) |
| 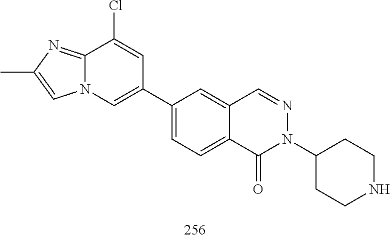 [SMILES: Cc1cn2cc(-c3ccc4c(=O)n(C5CCNCC5)ncc4c3)cc(Cl)c2n1] | 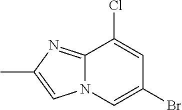 | 394 | (400 MHz, DMSO-d₆) δ 9.11 (d, J = 1.6 Hz, 1H), 8.50 (s, 1H), 8.34 (dd, J = 5.2, 3.3 Hz, 2H), 8.23 (dd, J = 8.4, 1.8 Hz, 1H), 7.93 (d, J = 1.6 Hz, 1H), 7.88 (s, 1H), 4.98 (tt, J = 11.7, 4.1 Hz, 1H), 3.14-3.06 (m, 2H), 2.66 (td, J = 12.4, 2.5 Hz, 2H), 2.40 (s, 3H), 1.89 (qd, J = 12.2, 4.1 Hz, 2H), 1.77-1.68 (m, 2H) |
| 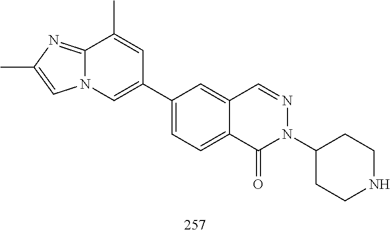 [SMILES: Cc1cn2cc(-c3ccc4c(=O)n(C5CCNCC5)ncc4c3)cc(C)c2n1] | 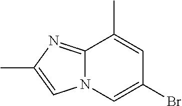 | 374 | (400 MHz, DMSO-d₆) δ 8.92 (d, J = 1.8 Hz, 1H), 8.51 (s, 1H), 8.33 (d, J = 8.4 Hz, 1H), 8.27 (d, J = 1.8 Hz, 1H), 8.19 (dd, J = 8.4, 1.8 Hz, 1H), 7.73 (d, J = 1.1 Hz, 1H), 7.52 (t, J = 1.6 Hz, 1H), 4.96 (tt, J = 11.7, 4.1 Hz, 1H), 3.12-3.04 (m, 2H), 2.63 (td, J = 12.5, 2.5 Hz, 2H), 2.55 (s, 3H), 2.37 (s, 3H), 1.87 (qd, J = 12.1, 4.1 Hz, 2H), 1.75-1.66 (m, 2H) |
| 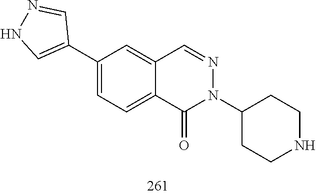 [SMILES: O=c1c2ccc(-c3cn[nH]c3)cc2cnn1C1CCNCC1] | 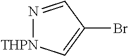 | 296 | (400 MHz, DMSO-d₆) δ 9.12 (d, J = 11.3 Hz, 1H), 8.89 (d, J = 11.4 Hz, 1H), 8.43 (s, 1H), 8.30 (s, 2H), 8.23 (d, J = 8.3 Hz, 1H), 8.18 (d, J = 1.7 Hz, 1H), 8.14 (dd, J = 8.3, 1.7 Hz, 1H), 5.19 (tt, J = 11.6, 4.0 Hz, 1H), 3.41 (d, J = 12.5 Hz, 2H), 3.22-3.07 (m, 2H), 2.21 (qd, J = 13.4, 4.1 Hz, 2H), 1.96 (dd, J = 14.1, 3.8 Hz, 2H) |
| 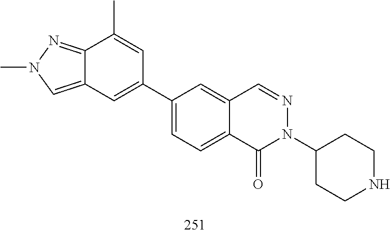 [SMILES: Cc1cc(-c2ccc3c(=O)n(C4CCNCC4)ncc3c2)cc2cn(C)nc12] | 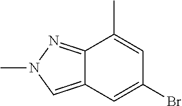 | 374 | (400 MHz, DMSO-d₆) δ 8.53 (s, 1H), 8.45 (s, 1H), 8.31 (d, J = 8.4 Hz, 1H), 8.27 (d, J = 1.8 Hz, 1H), 8.21 (dd, J = 8.4, 1.9 Hz, 1H), 8.01 (d, J = 1.7 Hz, 1H), 7.51 (t, J = 1.5 Hz, 1H), 4.99 (ddd, J = 11.7, 7.7, 4.1 Hz, 1H), 4.22 (s, 3H), 3.15- 3.07 (m, 2H), 2.68 (td, J = 12.5, 2.5 Hz, 2H), 2.60 (s, 3H), 1.90 (qd, J = 12.2, 4.1 Hz, 2H), 1.77-1.68 (m, 2H) |
| 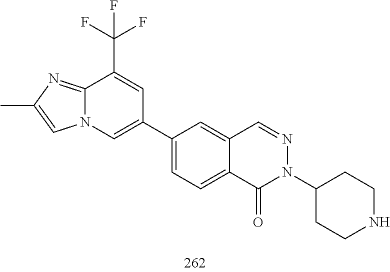 [SMILES: Cc1cn2cc(-c3ccc4c(=O)n(C5CCNCC5)ncc4c3)cc(C(F)(F)F)c2n1] | 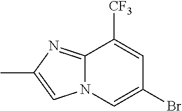 | 428 | (400 MHz, DMSO-d₆) δ 9.60 (s, 1H), 9.26 (d, J = 11.2 Hz, 1H), 9.16-9.08 (m, 1H), 8.57 (s, 1H), 8.47 (d, J = 1.9 Hz, 1H), 8.40 (d, J = 8.2 Hz, 2H), 8.32 (dd, J = 8.4, 1.9 Hz, 1H), 8.12 (s, 1H), 5.22 (ddt, J = 11.5, 7.6, 4.0 Hz, 1H), 3.41 (d, J = 12.4 Hz, 2H), 3.23-3.09 (m, 2H), 2.51 (s, 3H), 2.25 (qd, J = 13.3, 4.1 Hz, 2H), 2.03- 1.94 (m, 2H); ¹⁹F NMR (376 MHz, DMSO) δ −61.40, −61.63, −62.12 |
| 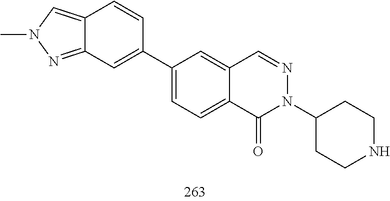 [SMILES: Cn1cc2ccc(-c3ccc4c(=O)n(C5CCNCC5)ncc4c3)cc2n1] | 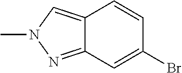 | 360 | (400 MHz, DMSO-d₆) δ 8.55 (s, 1H), 8.42 (s, 1H), 8.33 (dd, J = 5.1, 3.2 Hz, 2H), 8.25 (dd, J = 8.5, 1.7 Hz, 1H), 8.06 (d, J = 1.5 Hz, 1H), 7.88 (d, J = 8.7 Hz, 1H), 7.51 (dd, J = 8.8, 1.5 Hz, 1H), 4.98 (ddt, J = 11.5, 7.8, 4.1 Hz, 1H), 4.22 (s, 3H), 3.10 (d, J = 12.1 Hz, 2H), 2.72- 2.62 (m, 2H), 1.90 (qd, J = 12.3, 4.0 Hz, 2H), 1.77-1.68 (m, 2H) |

### Example 57

### Synthesis of Compound 283

### Synthesis of Intermediate B119

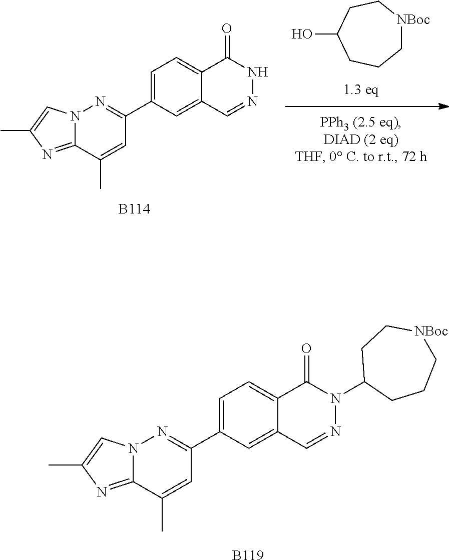 [SMILES: "B114" ; "B119" ; Cc1cn2nc(-c3ccc4c(=O)[nH]ncc4c3)cc(C)c2n1 ; Cc1cn2nc(-c3ccc4c(=O)n(C5CCCN(C(=O)OC(C)(C)C)CC5)ncc4c3)cc(C)c2n1 ; CC(C)(C)OC(=O)N1CCCC(O)CC1]

To a stirred solution of PPh₃ (450.2 mg, 1.72 mmol, 2.5 equiv) in THF (20 mL) was added DIAD (277.6 mg, 1.37 mmol, 2 equiv) dropwise at 0° C. under nitrogen atmosphere. To the reaction mixture was added 6-{2,8-dimethylimidazo[1,2-b]pyridazin-6-yl}-2H-phthalazin-1-one (200.0 mg, 0.69 mmol, 1.00 equiv) and tert-butyl 4-hydroxyazepane-1-carboxylate (192.1 mg, 0.89 mmol, 1.3 equiv) dropwise at 0° C. The resulting mixture was stirred for an additional 1 h at room temperature, then quenched with MeOH. The resulting mixture was concentrated under vacuum to give a residue. The residue was purified by Prep-TLC (CH₂Cl_{2/}MeOH=10:01) to afford tert-butyl 4-(6-{2,8-dimethylimidazo[1,2-b]pyridazin-6-yl}-1-oxophthala-zin-2-yl)azepane-1-carboxylate (60.0 mg, 17.89%) as an oil. LCMS (ES, m/z): 489 [M+H]⁺.

### Synthesis of Compound 283

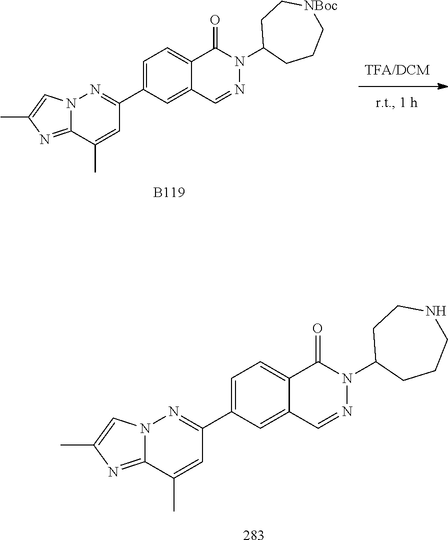 [SMILES: "B119" ; Cc1cn2nc(-c3ccc4c(=O)n(C5CCCN(C(=O)OC(C)(C)C)CC5)ncc4c3)cc(C)c2n1 ; Cc1cn2nc(-c3ccc4c(=O)n(C5CCCNCC5)ncc4c3)cc(C)c2n1 ; "283"]

A solution of tert-butyl 4-(6-{2,8-dimethylimidazo[1,2-b]pyridazin-6-yl}-1-oxophthalazin-2-yl)azepane-1-carboxylate (60.0 mg, 0.12 mmol, 1.00 equiv) in TFA (0.5 mL) was stirred for 1 h at room temperature. The resulting mixture was concentrated under vacuum to give a residue. The residue was purified by Prep-HPLC (Column: YMC-Actus Triart C18, 30*150 mm, 5 um; Mobile Phase A: water (10 mmol/L NH₄HCO₃), Mobile Phase B: ACN; Flow rate: 60 mL/min; Gradient: 5% B to 70% B in 8 min) to afford 2-(azepan-4-yl)-6-{2,8-dimethylimidazo[1,2-b]pyridazin-6-yl}phthalazin-1-one (9.1 mg, 18.9%) as a solid. LCMS (ES, m/z): 389 [M+H]⁺. ¹H NMR (400 MHz, DMSO-d₆) δ 8.59 — 8.51 (m, 2H), 8.47 (dd, J=8.4, 1.8 Hz, 1H), 8.40 (d, J=8.4 Hz, 1H), 8.06 (s, 1H), 7.72 (d, J=1.3 Hz, 1H), 5.21 (tt, J=9.7, 4.6 Hz, 1H), 3.49 (d, J=58.9 Hz, 1H), 2.98 — 2.93 (m, 1H), 2.92 — 2.75 (m, 2H), 2.67 (d, J=1.1 Hz, 3H), 2.45 (d, J=0.8 Hz, 3H), 2.33 (p, J=1.9 Hz, 2H), 1.94 — 1.89 (m, 1H), 1.89 — 1.77 (m, 2H), 1.64 (td, J=15.1, 14.3, 6.9 Hz, 1H).

### Example 58

### Synthesis of Compounds 244-246, 284-288, 297-303, and 307

### Synthesis of Intermediate B120

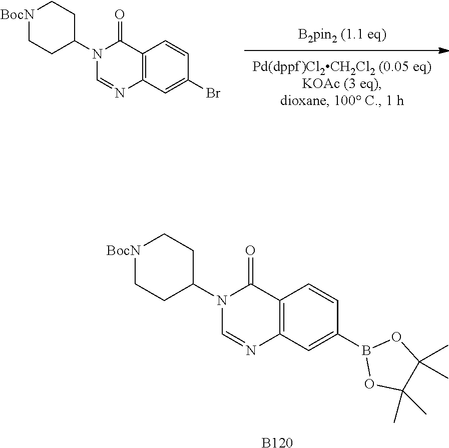 [SMILES: CC(C)(C)OC(=O)N1CCC(n2cnc3cc(Br)ccc3c2=O)CC1 ; "B120" ; CC(C)(C)OC(=O)N1CCC(n2cnc3cc(B4OC(C)(C)C(C)(C)O4)ccc3c2=O)CC1]

A mixture of tert-butyl 4-(7-bromo-4-oxoquinazolin-3-yl)piperidine-1-carboxylate (1.8 g, 4.41 mmol, 1.00 equiv), bis(pinacolato)diboron (1.23 g, 4.85 mmol, 1.10 equiv), KOAc (1.3 g, 13.23 mmol, 3 equiv), and Pd(dppf)Cl₂.CH₂Cl₂ (180.4 mg, 0.22 mmol, 0.05 equiv) in dioxane (18 mL) was stirred for 1 h at 100° C. under nitrogen atmosphere. The resulting mixture was filtered. LCMS (ES, m/z): 456 [M+H]⁺.

### Synthesis of Intermediate B121

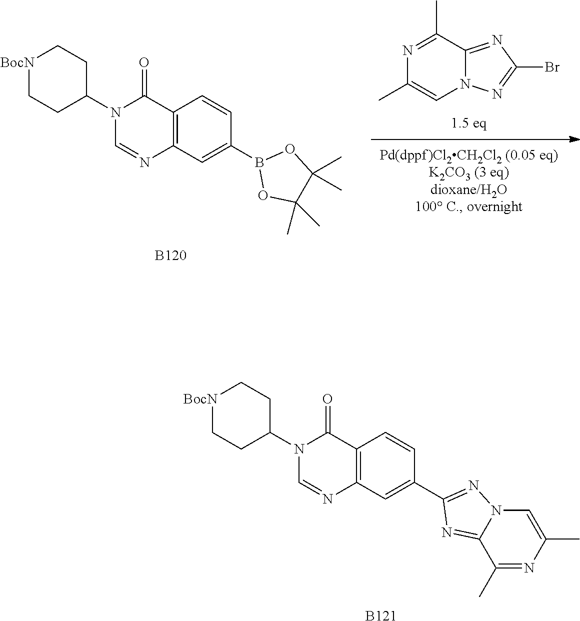 [SMILES: "B120" ; CC(C)(C)OC(=O)N1CCC(n2cnc3cc(B4OC(C)(C)C(C)(C)O4)ccc3c2=O)CC1 ; "B121" ; Cc1cn2nc(-c3ccc4c(=O)n(C5CCN(C(=O)OC(C)(C)C)CC5)cnc4c3)nc2c(C)n1 ; Cc1cn2nc(Br)nc2c(C)n1]

A mixture of tert-butyl 4-[4-oxo-7-(4,4,5,5-tetramethyl-1,3,2-dioxaborolan-2-yl)quinazolin-3-yl]piperidine-1-carboxylate (220.0 mg, 0.48 mmol, 1.00 equiv), 2-bromo-6,8-dimethyl-[1,2,4]triazolo[1,5-a]pyrazine (164.5 mg, 0.72 mmol, 1.5 equiv), Pd(dppf)Cl₂.CH₂Cl₂ (19.7 mg, 0.02 mmol, 0.05 equiv) and K₂CO₃ (200.3 mg, 1.45 mmol, 3 equiv) in dioxane (2 mL) and water (0.5 mL) was stirred overnight at 100° C. under nitrogen atmosphere. The resulting mixture was diluted with water (20 mL). The resulting mixture was extracted with EtOAc (3×20 mL). The combined organic layers were washed with brine (1×20 mL), dried over anhydrous Na₂SO₄. After filtration, the filtrate was concentrated under reduced pressure. The residue was purified by silica gel column chromatography, eluted with CH₂Cl₂/MeOH (1:0) to afford tert-butyl 4-(7-{6, 8-dimethyl-[1,2,4]triazolo[1,5-a]pyrazin-2-yl}-4-oxoquinazolin-3-yl)piperidine-1-carboxylate(220.0 mg, 82.3%) as a solid. LCMS (ES, m/z): 476 [M+H]⁺.

### Synthesis of Compound 287

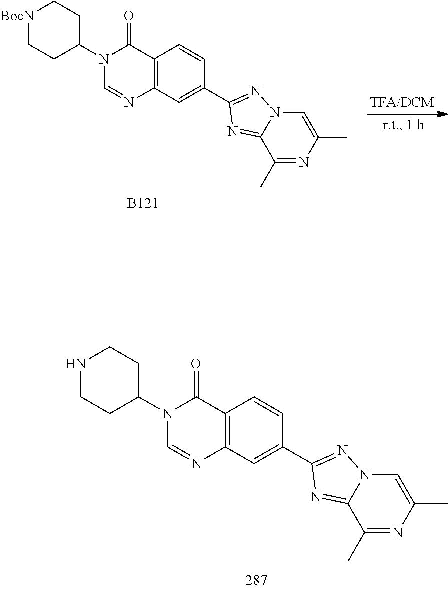 [SMILES: "B121" ; Cc1cn2nc(-c3ccc4c(=O)n(C5CCN(C(=O)OC(C)(C)C)CC5)cnc4c3)nc2c(C)n1 ; Cc1cn2nc(-c3ccc4c(=O)n(C5CCNCC5)cnc4c3)nc2c(C)n1]

A mixture of tert-butyl 4-(7-{6,8-dimethyl-[1,2,4]triazolo[1,5-a]pyrazin-2-yl}-4-oxoquinazolin-3-yl)piperidine-1-carboxylate (220.0 mg, 0.46 mmol, 1.00 equiv), DCM (3 mL) and TFA (0.75 mL) was stirred for 1 h at room temperature. The resulting mixture was concentrated under vacuum to give a residue. The residue was purified by Prep-HPLC (Column: YMC-Actus Triart C18, 30*150 mm, 5 um; Mobile Phase A: Water (10 mmol/L NH₄HCO₃), Mobile Phase B: ACN; Flow rate: 60 mL/min; Gradient: 5% B to 50% B in 8 min) to afford 7-{6,8-dimethyl-[1,2,4]triazolo[1,5-a]pyrazin-2-yl}-3-(piperidin-4-yl)quinazolin-4-one (19.6 mg, 11.17%) as a solid.

### Synthesis of Compound 288

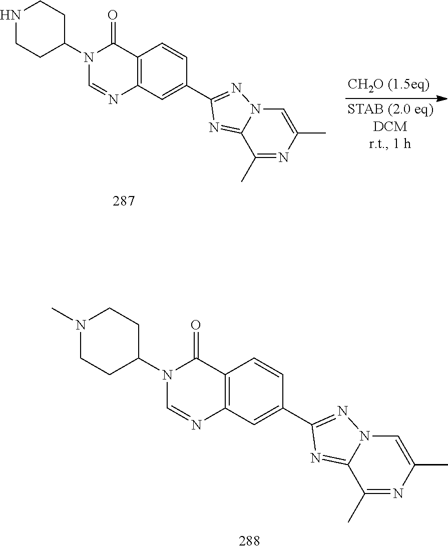

To a stirred mixture of 7-{6,8-dimethyl-[1,2,4]triazolo[1,5-a]pyrazin-2-yl}-3-(piperidin-4-yl)quinazolin-4-one (60.0 mg, 0.16 mmol, 1.00 equiv) and HCHO (48.0 mg, 1.60 mmol, 10 equiv) in DCM (3 mL) was added STAB (67.7 mg, 0.32 mmol, 2 equiv) dropwise at 0° C. The resulting mixture was stirred for 1 h at room temperature, then concentrated under vacuum to give a residue. The residue was purified by Prep-HPLC (Column: Xselect CSH OBD Column 30*150 mm Sum, n; Mobile Phase A: Water (10 mmol/L NH₄HCO₃), Mobile Phase B: ACN; Flow rate: 60 mL/min; Gradient: 5% B to 65% B in 8 min) to afford 7-{6,8-dimethyl-[1,2,4]triazolo[1,5-a]pyrazin-2-yl}-3-(1-methylpiperidin-4-yl)quinazolin-4-one (19.1 mg, 30.59%) as a solid.

Compounds 244-246, 284-288, 297-303, and 307 were prepared according to the procedures outlined herein. outlined in this Example 58 and generalized by Scheme F. The table below provides intermediates used in these procedures and final compound characterization data.

| | | LCMS | |
|---|---|---|---|
| | Coupling | (ESI, m/z) | |
| Compound No. and Structure | Reagent | [M + H]⁺ | ¹H NMR δ |
| 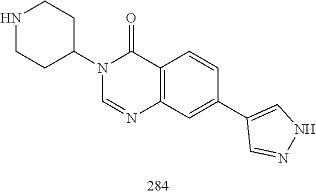 [SMILES: O=c1c2ccc(-c3cn[nH]c3)cc2ncn1C1CCNCC1] | 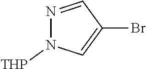 | 296 | (400 MHz, DMSO-d₆) δ 13.13 (s, 1H), 8.41 (s, 1H), 8.31 (s, 2H), 8.11 (d, J = 8.3 Hz, 1H), 7.92 (d, J = 1.6 Hz, 1H), 7.82 (dd, J = 8.3, 1.7 Hz, 1H), 4.68 (tt, J = 12.2, 3.9 Hz, 1H), 3.14-3.06 (m, 2H), 2.63 (td, J = 12.1, 2.4 Hz, 2H), 1.92 (qd, J = 12.0, 4.1 Hz, 2H), 1.82-1.73 (m, 2H) |
| 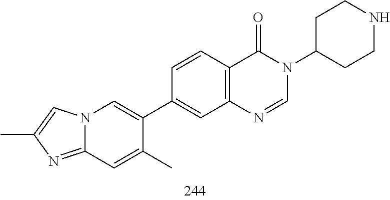 [SMILES: Cc1cn2cc(-c3ccc4c(=O)n(C5CCNCC5)cnc4c3)c(C)cc2n1] | 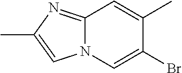 | 374 | (400 MHz, DMSO-d₆) δ 8.48 (d, J = 13.2 Hz, 2H), 8.23 (d, J = 8.2 Hz, 1H), 7.70 (d, J = 1.7 Hz, 1H), 7.64-7.57 (m, 2H), 7.39 (s, 1H), 4.72 (ddt, J = 12.3, 8.3, 4.0 Hz, 1H), 3.10 (d, J = 11.9 Hz, 2H), 2.62 (td, J = 12.2, 2.3 Hz, 2H), 2.33 (s, 3H), 2.25 (s, 3H), 1.93 (qd, J = 11.9, 4.0 Hz, 2H), 1.79 (t, J = 6.8 Hz, 2H) |
| 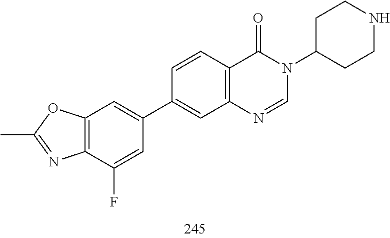 [SMILES: Cc1nc2c(F)cc(-c3ccc4c(=O)n(C5CCNCC5)cnc4c3)cc2o1] | 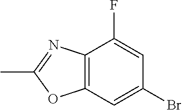 | 379 | (400 MHz, DMSO-d₆) δ 8.50 (s, 1H), 8.24 (d, J = 8.4 Hz, 1H), 8.10 (d, J = 1.4 Hz, 1H), 8.07 (d, J = 1.8 Hz, 1H), 7.97 (dd, J = 8.4, 1.9 Hz, 1H), 7.77 (dd, J = 11.5, 1.4 Hz, 1H), 4.70 (ddt, J = 12.2, 7.8, 4.0 Hz, 1H), 3.14-3.06 (m, 2H), 2.69 (s, 3H), 2.67-2.56 (m, 2H), 1.93 (qd, J = 12.0, 4.0 Hz, 2H), 1.83-1.75 (m, 2H). ¹⁹F NMR (376 MHz, DMSO- d6) δ −126.03 |
| 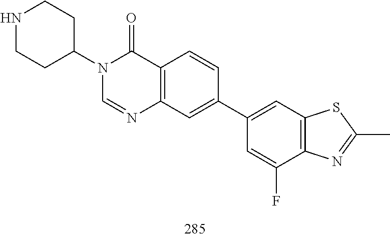 [SMILES: Cc1nc2c(F)cc(-c3ccc4c(=O)n(C5CCNCC5)cnc4c3)cc2s1] | 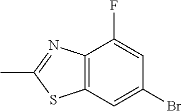 | 395 | (400 MHz, DMSO-d₆) δ 8.46 (s, 1H), 8.38 (d, J = 1.6 Hz, 1H), 8.23 (d, J = 8.3 Hz, 1H), 8.03 (d, J = 1.8 Hz, 1H), 7.95 (dd, J = 8.4, 1.8 Hz, 1H), 7.82 (dd, J = 12.0, 1.6 Hz, 1H), 4.70 (tt, J = 12.2, 4.0 Hz, 1H), 3.12-3.03 (m, 2H), 2.85 (s, 3H), 2.59 (td, J = 12.1, 2.5 Hz, 2H), 1.90 (qd, J = 12.1, 4.1 Hz, 2H), 1.82-1.74 (m, 2H). ¹⁹F NMR (376 MHz, DMSO) δ −122.62 |
| 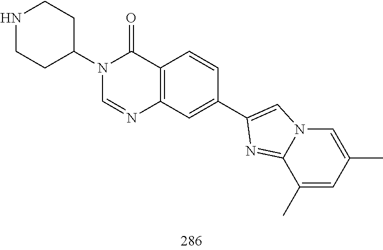 [SMILES: Cc1cc(C)c2nc(-c3ccc4c(=O)n(C5CCNCC5)cnc4c3)cn2c1] | 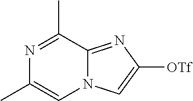 [SMILES: Cc1cn2cc(OS(=O)(=O)C(F)(F)F)nc2c(C)n1] | 375 | (400 MHz, DMSO-d₆) δ 8.71 (s, 1H), 8.46 (s, 1H), 8.28 (d, J = 1.5 Hz, 2H), 8.26-8.18 (m, 2H), 4.71 (ddt, J = 11.9, 7.6, 4.0 Hz, 1H), 3.14 (d, J = 12.5 Hz, 2H), 2.78 (s, 3H), 2.72-2.60 (m, 2H), 2.40 (s, 3H), 1.97 (qd, J = 12.1, 4.0 Hz, 2H), 1.86-1.77 (m, 2H) |
| 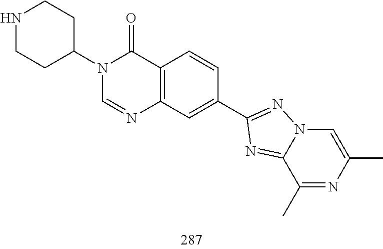 [SMILES: Cc1cn2nc(-c3ccc4c(=O)n(C5CCNCC5)cnc4c3)nc2c(C)n1] | 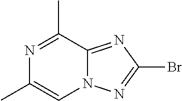 | 376 | (400 MHz, DMSO-d₆) δ 8.74 (t, J = 0.9 Hz, 1H), 8.44 (t, J = 1.1 Hz, 1H), 8.44 (s, 1H), 8.34 (d, J = 1.0 Hz, 2H), 4.71 (tt, J = 12.1, 4.0 Hz, 1H), 3.14 (dt, J = 13.0, 3.0 Hz, 2H), 2.88 (s, 3H), 2.67 (td, J = 11.7, 2.6 Hz, 2H), 2.55 (d, J = 1.0 Hz, 3H), 1.97 (qd, J = 12.0, 4.2 Hz, 2H), 1.89-1.80 (m, 2H) |
| 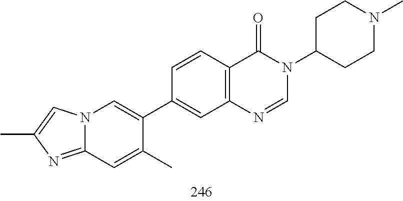 | 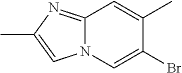 | 388 | (400 MHz, DMSO-d₆) δ 8.53 (s, 1H), 8.46 (s, 1H), 8.22 (d, J = 8.2 Hz, 1H), 7.70 (d, J = 1.7 Hz, 1H), 7.61 (d, J = 9.8 Hz, 2H), 7.39 (s, 1H), 4.62 (td, J = 11.9, 9.9, 5.8 Hz, 1H), 2.98-2.90 (m, 2H), 2.33 (s, 3H), 2.24 (d, J = 4.5 Hz, 6H), 2.17-2.01 (m, 4H), 1.85- 1.77 (m, 2H) |
| 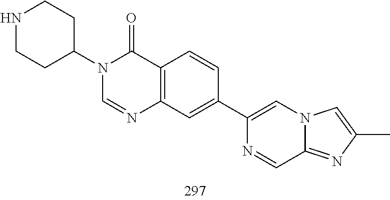 [SMILES: Cc1cn2cc(-c3ccc4c(=O)n(C5CCNCC5)cnc4c3)ncc2n1] | 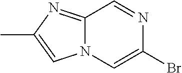 | 361 | (400 MHz, DMSO-d₆) δ 9.42 (d, J = 1.5 Hz, 1H), 9.07 (d, J = 1.5 Hz, 1H), 8.47 (s, 1H), 8.32 (d, J = 1.7 Hz, 1H), 8.30- 8.18 (m, 2H), 7.93 (d, J = 1.0 Hz, 1H), 4.72 (tt, J = 12.1, 3.9 Hz, 1H), 3.17-3.10 (m, 2H), 2.67 (td, J = 12.5, 12.1, 2.5 Hz, 2H), 2.46 (s, 3H), 1.97 (qd, J = 12.1, 4.0 Hz, 2H), 1.86-1.77 (m, 2H) |
| 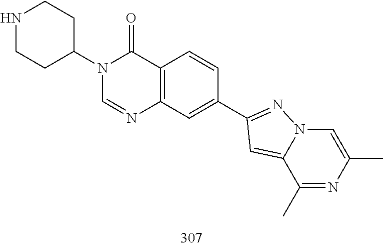 [SMILES: Cc1cn2nc(-c3ccc4c(=O)n(C5CCNCC5)cnc4c3)cc2c(C)n1] | 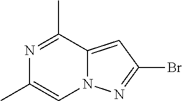 | 375 | (400 MHz, DMSO-d₆) δ 8.54 (s, 1H), 8.45 (s, 1H), 8.30- 8.23 (m, 2H), 8.19 (dd, J = 8.3, 1.6 Hz, 1H), 7.71 (d, J = 1.0 Hz, 1H), 4.73 (tt, J = 12.1, 3.9 Hz, 1H), 3.23-3.15 (m, 2H), 2.80-2.66 (m, 5H), 2.44 (d, J = 1.0 Hz, 3H), 2.02 (qd, J = 12.2, 4.0 Hz, 2H), 1.90-1.82 (m, 2H) |
| 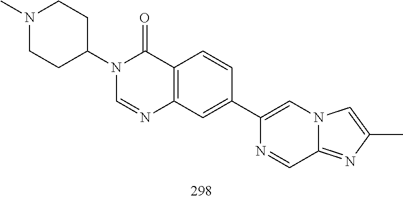 | 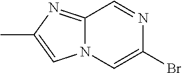 | 375 | (400 MHz, DMSO-d₆) δ 9.42 (d, J = 1.5 Hz, 1H), 9.07 (d, J = 1.4 Hz, 1H), 8.52 (s, 1H), 8.32 (d, J = 1.6 Hz, 1H), 8.29- 8.18 (m, 2H), 7.93 (s, 1H), 4.61 (ddt, J = 11.7, 7.8, 3.8 Hz, 1H), 2.93 (dd, J = 9.2, 2.5 Hz, 2H), 2.46 (s, 3H), 2.23 (s, 3H), 2.12-2.00 (m, 4H), 1.86-1.77 (m, 2H) |
| 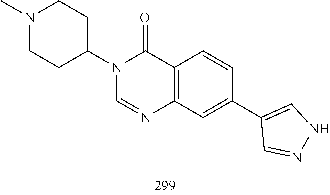 [SMILES: CN1CCC(n2cnc3cc(-c4cn[nH]c4)ccc3c2=O)CC1] | 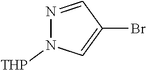 | 310 | (400 MHz, DMSO-d₆) δ 13.14 (s, 1H), 8.44 (s, 2H), 8.10 (d, J = 8.3 Hz, 2H), 7.91 (d, J = 1.7 Hz, 1H), 7.82 (dd, J = 8.3, 1.7 Hz, 1H), 4.57 (tt, J = 11.9, 4.0 Hz, 1H), 2.92 (dd, J = 9.0, 2.5 Hz, 2H), 2.22 (s, 3H), 2.18-1.99 (m, 4H), 1.82-1.75 (m, 2H) |
| 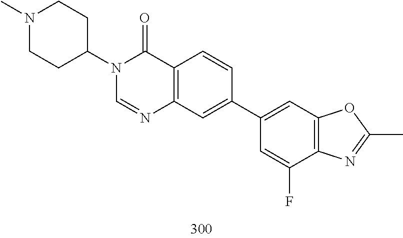 | 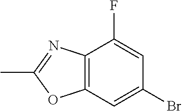 | 393 | (400 MHz, DMSO-d₆) δ 8.53 (s, 1H), 8.23 (d, J = 8.4 Hz, 1H), 8.08 (dd, J = 14.0, 1.7 Hz, 2H), 7.96 (dd, J = 8.4, 1.9 Hz, 1H), 7.76 (dd, J = 11.4, 1.5 Hz, 1H), 4.59 (td, J = 11.8, 3.9 Hz, 1H), 2.97-2.90 (m, 2H), 2.69 (s, 3H), 2.23 (s, 3H), 2.10 (ddd, J = 22.1, 11.7, 2.7 Hz, 4H), 1.81 (dd, J = 10.1, 4.0 Hz, 2H); ¹⁹F NMR (376 MHz, DMSO) δ −126.03 |
| 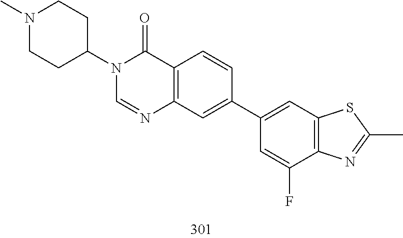 | 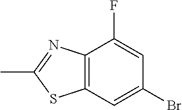 | 409 | (400 MHz, DMSO-d₆) δ 8.45 (s, 1H), 8.36 (d, J = 1.6 Hz, 1H), 8.26 (d, J = 8.3 Hz, 1H), 8.03 (d, J = 1.8 Hz, 1H), 7.94 (dd, J = 8.4, 1.9 Hz, 1H), 7.78 (dd, J = 12.1, 1.7 Hz, 1H), 4.65-4.54 (m, 1H), 2.99- 2.92 (m, 2H), 2.87 (s, 3H), 2.26 (s, 3H), 2.23-2.06 (m, 4H), 1.89-1.81 (m, 2H); ¹⁹F NMR (376 MHz, DMSO) δ −122.50 |
| 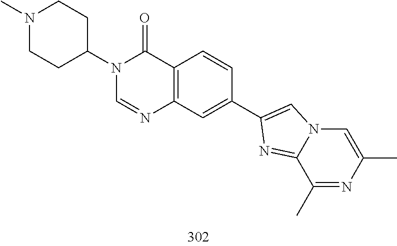 | 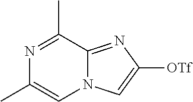 [SMILES: Cc1cn2cc(OS(=O)(=O)C(F)(F)F)nc2c(C)n1] | 389 | (400 MHz, DMSO-d₆) δ 8.71 (s, 1H), 8.51 (s, 1H), 8.27 (d, J = 1.6 Hz, 2H), 8.26-8.16 (m, 2H), 4.65-4.54 (m, 1H), 2.93 (dd, J = 11.1, 3.4 Hz, 2H), 2.78 (s, 3H), 2.40 (d, J = 1.0 Hz, 3H), 2.23 (s, 3H), 2.20-2.01 (m, 4H), 1.85- 1.77 (m, 2H) |
| 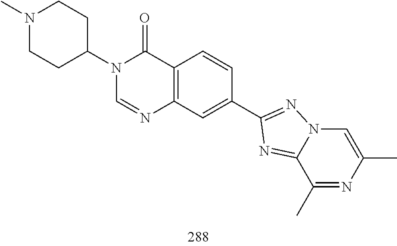 | 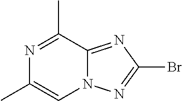 | 390 | (400 MHz, DMSO-d₆) δ 8.76 (s, 1H), 8.47 (s, 1H), 8.45 (d, J = 1.4 Hz, 1H), 8.34 (s, 2H), 4.65-4.54 (m, 1H), 2.99- 2.91 (m, 2H), 2.88 (s, 3H), 2.55 (s, 3H), 2.27 (s, 3H), 2.22-2.07 (m, 4H), 1.90- 1.82 (m, 2H) |
| 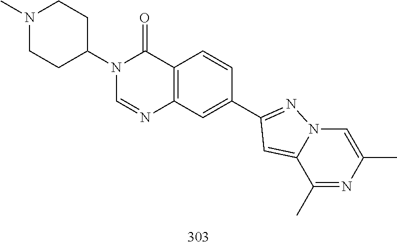 | 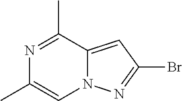 | 389 | (400 MHz, DMSO-d₆) δ 8.44 (d, J = 8.1 Hz, 2H), 8.29- 8.23 (m, 2H), 8.16 (dd, J = 8.5, 1.6 Hz, 1H), 7.59 (d, J = 1.0 Hz, 1H), 4.64-4.53 (m, 1H), 2.95 (dd, J = 8.4, 2.6 Hz, 2H), 2.74 (s, 3H), 2.46 (d, J = 1.0 Hz, 3H), 2.26 (s, 3H), 2.23-2.06 (m, 4H), 1.89- 1.80 (m, 2H) |

### Example 59

### Synthesis of Compound 241

### Synthesis of Intermediate B122

Tert-butyl 4-(3-(2,8-dimethylimidazo[1,2-b]pyridazin-6-yl)-4-oxo-3,4-dihydroquinazolin yl)piperazine-1-carboxylate (Intermediate B 122) was prepared using the procedure described in Example 2, where in the first step (i.e. for the preparation of B16) 2-methyl-2H-indazol-5-amine is substituted with 2,8-dimethylimidazo[1,2-b]pyridazin-6-amine, and in the third step (i.e. for the preparation of 152) 1-methylpiperazine is substituted with tert-butyl piperazine-1-carboxylate. Intermediate B122 was thus obtained as a solid. LCMS (ES, m/z): 476.3 [M+H]⁺. sp Synthesis of Compound 241

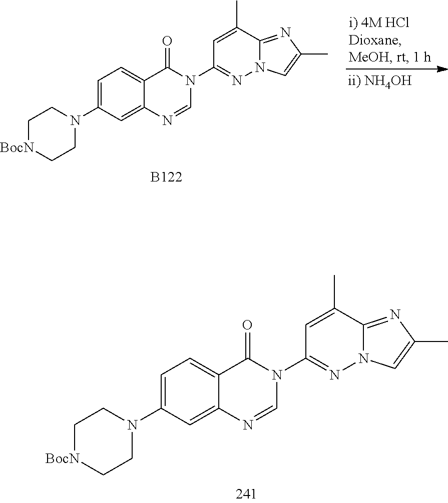 [SMILES: "B122" ; Cc1cn2nc(-n3cnc4cc(N5CCN(C(=O)OC(C)(C)C)CC5)ccc4c3=O)cc(C)c2n1 ; Cc1cn2nc(-n3cnc4cc(N5CCN(C(=O)OC(C)(C)C)CC5)ccc4c3=O)cc(C)c2n1]

To a solution of tert-butyl 4-(3-(2,8-dimethylimidazo[1,2-b]pyridazin-6-yl)-4-oxo-3,4-dihydroquinazolin-7-yl)piperazine-1-carboxylate (30 mg, 0.06 mmol) in methanol (1.0 mL) and DCM (0.5 mL) was added 4M HCl in dioxane (2.0 mL). The reaction mixture was stirred at room temperature for 1 h. The volatiles were evaporated under reduced pressure, ethyl acetate (5 mL) was added, and a precipitate formed. The suspension was centrifuged, and the supernatant decanted. The solid was washed with ethyl acetate, the suspension centrifuged, the supernatant decanted, and the solid dried. The solid was dissolved in water (1 mL), basified with NH₄OH (10%, pH 1 to pH 10). A precipitate formed, and the suspension was centrifuged, the supernatant decanted, and the solid washed with water (2×1 mL). The resulting suspension was centrifuged, the supernatant decanted, and the solid lyophilized to afford 3-(2,8-dimethylimidazo[1,2-b]pyridazin-6-yl)-7-(piperazin-1-yl)quinazolin-4(3H)-one (12 mg, 51%). LCMS (ES, m/z): 376.2 [M+H]⁺. ¹H NMR (CH₂Cl₂-d₂, 400 MHz): δ_{H} 8.26 (1H, s), 8.16 (1H, d, J=9.0 Hz), 7.77 (1H, s), 7.24 (1H, s), 7.14 (1H, dd, J=9.1, 2.6 Hz), 7.08 (1H, d, J=2.5 Hz), 3.53 (4H, t, J=5.1 Hz), 3.15 (4H, t, J=4.9 Hz), 2.69 (3H, s), 2.50 (3H, s).

### Example 60

### Synthesis of Compounds 243 and 242

### Synthesis of Intermediate B125

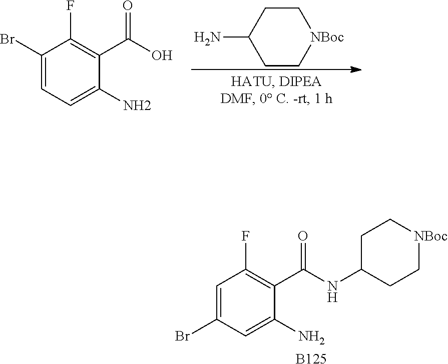

To a mixture of 2-amino-5-bromo-6-fluorobenzoic acid (1.00 g, 4.27 mmol) and tert-butyl 4-aminopiperidine-1-carboxylate (899 mg, 4.49 mmol) in DMF (20.0 mL) were sequentially added DIPEA (2.23 mL, 12.8 mmol) and HATU (1.95 g, 5.13 mmol). The reaction mixture was stirred 0° C. for 1 h. Ethyl acetate (100 mL) and NH₄Cl (sat) (100 mL) were added. The organic layer was separated, washed with NH₄Cl (sat) (50 mL), NaHCO₃ (sat) (50 mL), and brine (50 mL), dried over MgSO₄, filtered, and the filtrate concentrated under reduced pressure to afford tert-butyl 4-(2-amino-4-bromo-6-fluorobenzamido)piperidine-1-carboxylate (1760 mg, 99%) as a solid. LCMS (ES, m/z): 438.0, 440.0 [M+Na]⁺.

### Synthesis of Intermediate B126

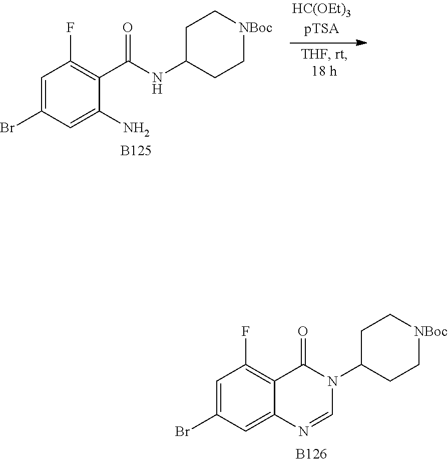 [SMILES: "B125" ; "B126" ; CC(C)(C)OC(=O)N1CCC(NC(=O)c2c(N)cc(Br)cc2F)CC1 ; CC(C)(C)OC(=O)N1CCC(n2cnc3cc(Br)cc(F)c3c2=O)CC1]

To a solution of tert-butyl 4-(2-amino-4-bromo-6-fluorobenzamido)piperidine-1-carboxylate (1.70 g, 4.1 mmol) in THF (40 mL) was added triethyl orthoformate (6.05 g, 40.8 mmol) and pTSA (0.08g, 0.41 mmol). The reaction mixture was stirred at room temperature for 18 hrs. Ethyl acetate (200 mL) was added, and the organic layer was washed with NaHCO₃ (sat) (2×50 mL) and brine (50 mL), dried over MgSO₄, filtered, and the filtrate concentrated under reduced pressure to afford tert-butyl 4-(7-bromo-5-fluoro-4-oxoquinazolin-3(4H)-yl)piperidine-1-carboxylate (1.7 g, 98%) as a solid. LCMS (ES, m/z) : 425.1, 427.1 [M+H]⁺.

### Synthesis of Intermediate B127

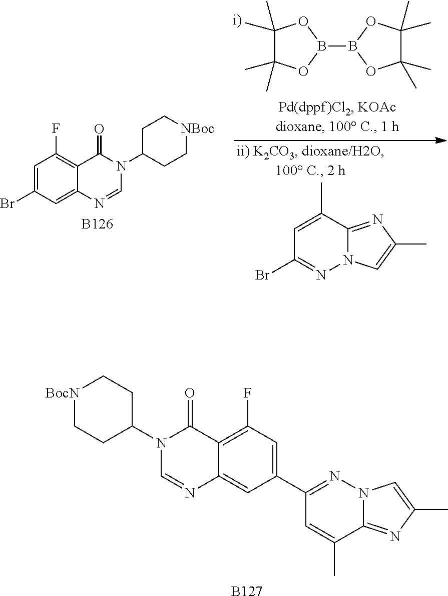

A mixture of tert-butyl 4-(7-bromo-5-fluoro-4-oxoquinazolin-3(4H)-yl)piperidine-1-carboxylate (200 mg, 0.47 mmol), bis(pinacolato)diboron (132 mg, 0.52 mmol), PdCl₂(dppf) (34 mg, 0.05 mmol), and KOAc (140 mg, 1.4 mmol) in dioxane (3.0 mL) was heated to 100° C. under a nitrogen atmosphere for 1 h, then cooled to room temperature. A solution of 6-bromo-2,8-dimethylimidazo[1,2-b]pyridazine (106 mg, 0.47 mmol) in dioxane (5.0 mL), K₂CO₃ (259 mg, 1.9 mmol) and water (1.0 mL) were sequentially added. The reaction mixture was heated at 100 ° C. for 2 h, then cooled to room temperature. The reaction mixture was diluted with ethyl acetate (50 mL), and washed with water (25 mL) and brine (25 mL), The organic layer was separated, dried over MgSO₄, filtered, and the filtrate concentrated under reduced pressure to give a residue. The residue was purified by normal phase flash chromatography using a gradient from 10 to 50% (EtOAc/10% MeOH)/DCM to afford tert-butyl 4-(7-(2,8-dimethylimidazo[1,2-b]pyridazin-6-yl)-5-fluoro-4-oxoquinazolin-3(4H)-yl)piperidine-1-carboxylate (190 mg, 82%) as a solid. LCMS (ES, m/z): 493.2 [M+H]⁺

### Synthesis of Compound 243

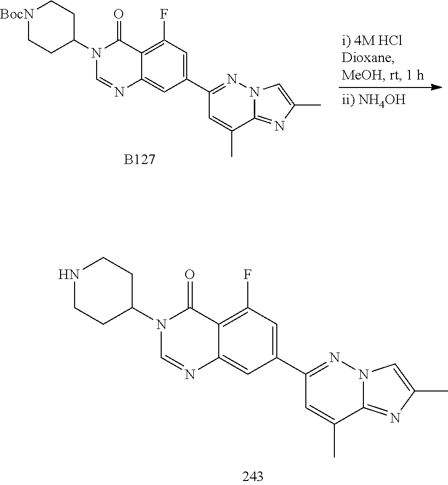 [SMILES: "B127" ; Cc1cn2nc(-c3cc(F)c4c(=O)n(C5CCN(C(=O)OC(C)(C)C)CC5)cnc4c3)cc(C)c2n1 ; Cc1cn2nc(-c3cc(F)c4c(=O)n(C5CCNCC5)cnc4c3)cc(C)c2n1]

To a solution of tert-butyl 4-(7-(2,8-dimethylimidazo[1,2-b]pyridazin-6-yl)-5-fluoro-4-oxoquinazolin-3(4H)-yl)piperidine-1-carboxylate (190 mg, 0.39 mmol) in methanol (2.0 mL) and DCM (1.0 mL) was added 4M HCl in dioxane (5.0 mL). The reaction mixture was stirred at room temperature for 1 h. The volatiles were evaporated under reduced pressure, ethyl acetate (5 mL) was added, and a precipitate formed. The suspension was centrifuged, the supernatant decanted, and the solid washed with ethyl acetate (2 mL). The suspension was centrifuged, the supernatant decanted, and the solid dried to yield the HCl salt of tert-butyl 44742,8-dimethylimidazo[1,2-b]pyridazin-6-yl)-5-fluoro-4-oxoquinazolin-3(4H)-yl)piperidine carboxylate. The salt was taken up in water (3 mL), basified with NH₄OH (10%, pH1 to pH 10). The aqueous layer was extracted with DCM (2×10 mL), and the combined organic layers were dried over MgSO₄, filtered, and the filtrate concentrated under reduced pressure to yield the product as a solid which was taken up in a mixture of acetonitrile and water (1:1, 10 mL), then lyophilized to afford 7-(2,8-dimethylimidazo[1,2-b]pyridazin-6-yl)-5-fluoro-3-(piperidin-4-yl)quinazolin-4(3H)-one (120 mg, 79%). LCMS (ES, m/z): 393.2 [M+H]⁺. ¹H NMR (CH₂Cl₂-d₂, 400 MHz): δ_{H} 8.21 (1H, s), 8.05 (1H, s), 7.85 (1H, d, J=12.0 Hz), 7.82 (1H, s), 7.38 (1H, s), 4.96 (1H, m), 3.40 (2H, d, J=12.5 Hz), 2.93 (2H, t, J=10.3 Hz), 2.70 (3H, s), 2.51 (3H, s), 2.06 (4H, t, J=7.7 Hz).

### Synthesis of Compound 242

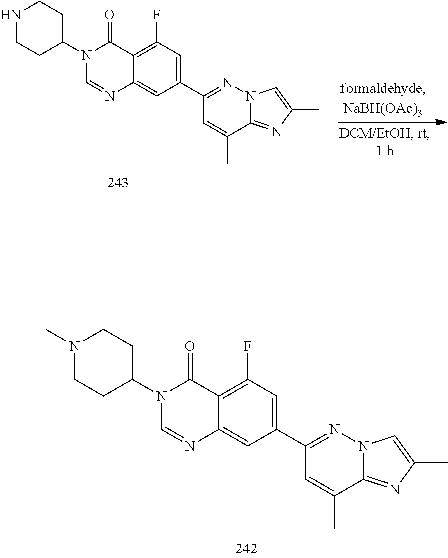

A mixture of 7-(2,8-dimethylimidazo[1,2-b]pyridazin-6-yl)-5-fluoro-3-(piperidin-4-yl)quinazolin-4(3H)-one (46 mg, 0.12 mmol), and formaldehyde (37% in water, 18 mg, 0.048 mL, 0.59 mmol) in DCM (3 mL) and ethanol (1 mL) was stirred at room temperature for 1 hr. Then NaBH(OAc)₃ (149 mg, 0.70 mmol) was added, and the reaction mixture was stirred at room temperature for 1 hr. The reaction mixture was diluted with DCM (50 mL), and washed with saturated NaHCO₃ (2×50 mL) and brine (50 mL). The organic layer was dried over MgSO₄, filtered, and the filtrate concentrated under reduced pressure to give a residue. The residue was purified by normal phase chromatography using a gradient from 10 to 50% (ethyl acetate/10% MeOH)/DCM with 1% Et3N additive to afford 7-(2,8-dimethylimidazo[1,2-b]pyridazin-6-yl)-5-fluoro-3-(1-methylpiperidin-4-yl)quinazolin-4(3H)-one (3.8 mg, 8%) as a solid. LCMS (ES, m/z): 407.2 [M+H]⁺. ¹H NMR (CH₂Cl₂-d₂+CD₃OD, 400 MHz): δ_{H} 8.45 (1H, s), 8.11 (1H, s), 7.84-7.89 (2H, m), 7.53 (1H, s), 5.14 (1H, t, J=4.0 Hz), 3.67-3.72 (2H, m), 3.07-3.15 (2H, m), 2.86 (3H, s), 2.77 (2H, m), 2.73 (3H, s), 2.54 (3H, s), 2.17-2.21 (2H, m).

### Example 61

### Synthesis of Compounds 289 and 290

### Synthesis of Compound 289

Compound 289 was prepared according to the procedure described for the preparation of Compound 243 where in the first step (i.e. for preparation of Intermediate B125, Example 60) 2-amino-5-bromo-6-fluorobenzoic acid is substituted with 2-amino-5-bromo-3-fluorobenzoic acid. 7-(2,8-dimethylimidazo[1,2-b]pyridazin-6-yl)-8-fluoro-3-(piperidin-4-yl)quinazolin-4(3H)-one was thus obtained as a solid. LCMS (ES, m/z): 393.2 [M+H]⁺.

### Synthesis of Compound 290

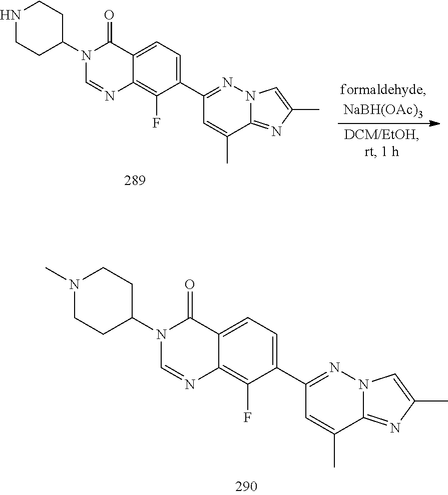

A mixture of 7-(2,8-dimethylimidazo[1,2-b]pyridazin-6-yl)-8-fluoro-3-(piperidin-4-yl)quinazolin-4(3H)-one (36 mg, 0.09 mmol), and formaldehyde (37% in water, 14 mg, 0.037 mL, 0.46 mmol) in DCM (3 mL) and ethanol (1 mL) was stirred at room temperature for 1 hr. Then NaBH(OAc)₃ (117 mg, 0.55 mmol) was added, and the reaction mixture was stirred at room temperature for an additional 1 hr. The resulting mixture was diluted with DCM (50 mL), washed with saturated NaHCO₃ (2×50 mL) and brine (50 mL). The organic layer was dried over MgSO₄, filtered, and the filtrate concentrated under reduced pressure to give a residue. The residue was purified by normal phase chromatography using a gradient from 10 to 50% (EtOAc/10% MeOH)/DCM with 1% Et₃N additive to afford 7-(2,8-dimethylimidazo[1,2-b]pyridazin-6-yl)-8-fluoro-3-(1-methylpiperidin-4-yl)quinazolin-4(3H)-one (13 mg, 35%) as a solid. LCMS (ES, m/z): 407.2 [M+H]⁺. ¹H NMR (CH₂Cl₂-d₂ +CD₃OD, 400 MHz): δ_{H} 8.45 (1H, s), 8.11 (1H, s), 7.84-7.89 (2H, m), 7.53 (1H, s), 5.14 (1H, t, J=4.0 Hz), 3.67-3.72 (2H, m), 3.07-3.15 (2H, m), 2.86 (3H, s), 2.77 (2H, m), 2.73 (3H, s), 2.54 (3H, s), 2.17-2.21 (2H, m).

### Example 62

### Synthesis of Compound 276

### Synthesis of Intermediate B123

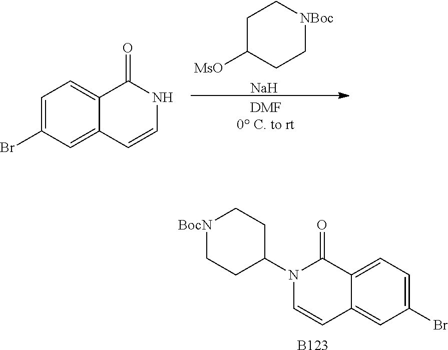

To a solution of 6-bromoisoquinolin-1(2H)-one (130 mg, 0.58 mmol) in DMF (5.8 mL) at 0° C. under nitrogen atmosphere was added NaH 60% (34.8 mg, 0.87 mmol). The reaction mixture was stirred at 0° C. for 1 h. To the reaction mixture was added tert-butyl 4-((methylsulfonyl)oxy)piperidine-1-carboxylate, and the resulting mixture was stirred at room temperature overnight. Ethyl acetate (50 mL) and NH₄Cl (sat) (50 ml) were added. The organic layer was separated, washed with NH₄Cl (sat) (50 ml), NaHCO₃ (sat) (50 ml) and brine (50 mL), then dried over MgSO₄, and filtered. The filtrate was concentrated under reduced pressure to give a residue. The residue was purified by normal phase chromatography, eluted with 20 to 100% ethyl acetate/hexanes to afford tert-butyl 4-(6-bromo-1-oxoisoquinolin-2(1H)-yl)piperidine-1-carboxylate (43 mg, 18%) as a solid. LCMS (ES, m/z): 428.7, 430.7 [M+Na]⁺.

### Synthesis of Intermediate B124

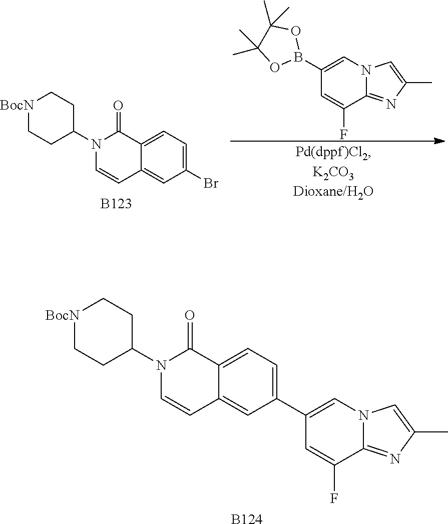

A suspension of tert-butyl 4-(6-bromo-1-oxoisoquinolin-2(1H)-yl)piperidine-1-carboxylate (43 mg, 0.11 mmol) and 8-fluoro-2-methyl-6-(4,4,5,5-tetramethyl-1,3,2-dioxaborolan-2-yl)imidazo[1,2-a]pyridine (32 mg, 0.12 mmol) in dioxane (1 mL) and water (0.2 mL) was degassed with argon. To the reaction mixture was added K₂CO₃ (46 mg, 0.33 mmol), followed by Pd(dppf)Cl2-DCM (10 mg, 0.012 mmol). The resulting mixture was stirred at 80° C. under an argon atmosphere for 3 h. The reaction mixture was partitioned between ethyl acetate (170 mL) and water (50 mL). The organic layer was washed with water (1×30 mL), dried over Na₂SO₄, filtered, and the filtrate concentrated in vacuo to give an oil which was purified on a silica gel chromatography, eluted with ethyl acetate/hexanes from 60 to 100% to afford tert-butyl 4-(6-(8-fluoro-2-methylimidazo[1,2-a]pyridin-6-yl)-1-oxoisoquinolin-2(1H)-yl)piperidine-1-carboxylate as a solid. LCMS (ES, m/z): 476.8 [M+H]⁺.

### Synthesis of Compound 276

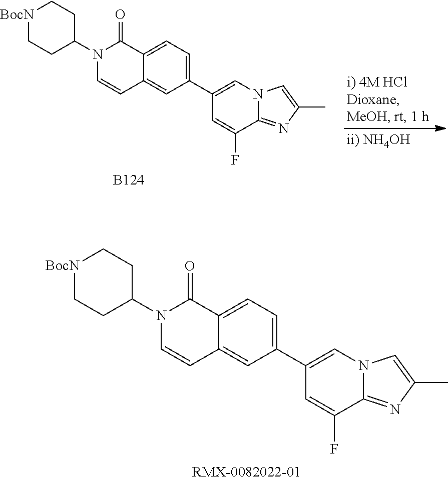 [SMILES: "B124" ; Cc1cn2cc(-c3ccc4c(=O)n(C5CCN(C(=O)OC(C)(C)C)CC5)ccc4c3)cc(F)c2n1 ; Cc1cn2cc(-c3ccc4c(=O)n(C5CCN(C(=O)OC(C)(C)C)CC5)ccc4c3)cc(F)c2n1]

To a solution of tert-butyl 4-(6-(8-fluoro-2-methylimidazo[1,2-a]pyridin-6-yl)-1-oxoisoquinolin-2(1H)-yl)piperidine-1-carboxylate (34 mg, 0.07 mmol) in methanol (1.0 mL) and DCM (0.5 mL) was added 4M HCl in dioxane (2.0 mL). The reaction mixture was stirred at room temperature for 1 h. The volatiles were evaporated under reduced pressure to yield a residue which was dissolved in water (2 mL), neutralized with 10% ammonium hydroxide (1 mL) and extracted with DCM (2×5 mL). The combined organic layers were washed with water (2×3 mL) and concentrated in vacuo to afford 6-(8-fluoro-2-methylimidazo[1,2-a]pyridin-6-yl)-2-(piperidin-4-yl)isoquinolin-1(2H)-one (16 mg, 59%). LCMS (ES, m/z): 377.2 [M+H]⁺. ¹H NMR (CH₂Cl₂-d₂, 400 MHz): δ_{H} 8.47 (1H, d, J=8.3 Hz), 8.25 (1H, d, J=1.5 Hz), 7.69 (1H, s), 7.66 (1H, dd, J=8.4, 1.8 Hz), 7.54 (1H, d, J=3.0 Hz), 7.29 (1H, d, J=7.6 Hz), 7.23 (1H, dd, J=11.7, 1.5 Hz), 6.61 (1H, d, J=7.5 Hz), 5.07-5.10 (1H, m), 3.22-3.26 (2H, m), 2.82-2.89 (2H, m), 2.48 (3H, s), 1.88-1.92 (2H, m), 1.77-1.83 (2H, m).

### Example 63

### Synthesis of Compound 291

### Synthesis of Intermediate B128

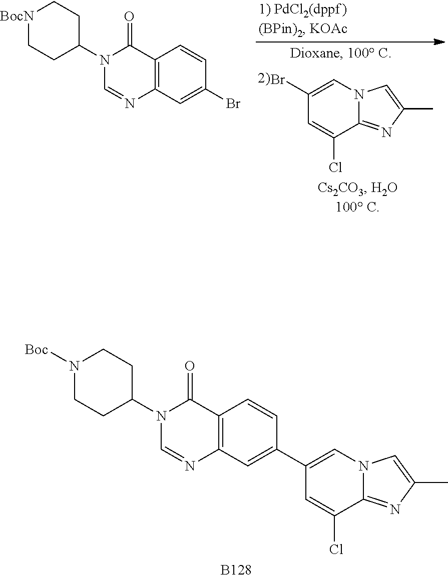 [SMILES: CC(C)(C)OC(=O)N1CCC(n2cnc3cc(Br)ccc3c2=O)CC1 ; Cc1cn2cc(Br)cc(Cl)c2n1 ; "B128" ; Cc1cn2cc(-c3ccc4c(=O)n(C5CCN(C(=O)OC(C)(C)C)CC5)cnc4c3)cc(Cl)c2n1]

Tert-butyl 4-(7-bromo-4-oxoquinazolin-3(4H)-yl)piperidine-1-carboxylate (118 mg, 0.29 mmol), bis(pinacolato)diboron (116 mg, 0.45 mmol), PdCl₂(dppf) (30 mg, 0.040 mmol), and potassium acetate (109 mg, 1.10 mmol) were dissolved in dioxane (2.0 mL), and the solution was bubbled with argon for 10 minutes. The reaction mixture was heated at 100° C. for 1 hour, then cooled. To the reaction mixture was added 6-bromo-8-chloro-2-methylimidazo[1,2-a]pyridine (109 mg, 0.44 mmol) in dioxane (1 mL), followed by K₂CO₃ (311 mg, 2.25 mmol) in water (340 μL). The reaction mixture was heated at 100° C. for 2 hours. Upon completion, the reaction mixture was diluted with ethyl acetate (25 mL), and washed with saturated NaHCO₃ (20 mL) and brine (2×20 mL). The organic phase was then filtered under vacuum, dried over Na₂SO₄, and concentrated in vacuo to give a residue. The residue was purified by flash chromatography on a silica gel column using a gradient of 70-100% ethyl acetate in hexane. Selected fractions were combined and evaporated under reduced pressure to afford tert-butyl 4-(7-(8-chloro-2-methylimidazo[1,2-a]pyridin-6-yl)-4-oxoquinazolin-3(4H)-yl)piperidine-1-carboxylate (107 mg, 75%) as a solid. LCMS (ES, m/z): 494.2 [M+H]⁺.

### Synthesis of Compound 291

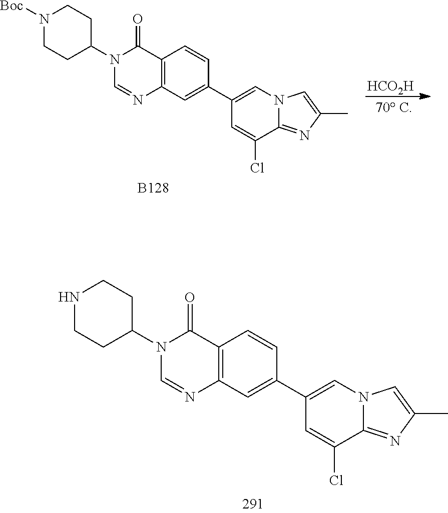 [SMILES: "B128" ; Cc1cn2cc(-c3ccc4c(=O)n(C5CCN(C(=O)OC(C)(C)C)CC5)cnc4c3)cc(Cl)c2n1 ; Cc1cn2cc(-c3ccc4c(=O)n(C5CCNCC5)cnc4c3)cc(Cl)c2n1 ; "HCO2H"]

A mixture of tert-butyl 4-(7-(8-chloro-2-methylimidazo[1,2-a]pyridin-6-yl)-4-oxoquinazolin-3(4H)-yl)piperidine-1-carboxylate (84 mg, 0.17 mmol) and formic acid (5 mL) was stirred vigorously at 70° C. for 2 hours. The reaction mixture was concentrated in vacuo to give a residue. The residue was purified by flash chromatography on a C18 column using a gradient of 5-70% MeCN in water with 0.1% formic acid additive. Selected fractions were combined, concentrated under reduced pressure, neutralized with (NH₄)₂CO_{3,} and lyophilized to afford 7-(8-chloro-2-methylimidazo[1,2-c]pyridin-6-yl)-3-(piperidin-4-yl)quinazolin-4(3H)-one (59 mg, 88%) as a solid. LCMS (ES, m/z): 394.1 [M+H]⁺. ¹H NMR (DMSO-d₆, 300 MHz): δ 9.09 (1H, s), 8.48 (1H, s), 8.22 (1H, d, J=8.4 Hz), 8.03 (1H, d, J=1.8 Hz), 7.89-7.92 (2H, m), 7.84 (1H, d, J=1.1 Hz), 4.68 (1H, t,=12.7 Hz), 3.08 (2H, d,=12.3 Hz), 2.60 (2H, t,=12.0 Hz), 2.38 (3H, s), 1.84-1.97 (2H, m), 1.76 (2H, d, J=11.9 Hz).

### Example 64

### Synthesis of Compound 292

### Synthesis of Intermediate B129

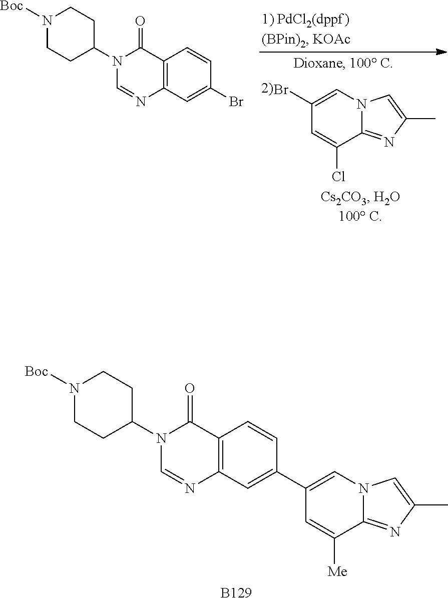 [SMILES: CC(C)(C)OC(=O)N1CCC(n2cnc3cc(Br)ccc3c2=O)CC1 ; Cc1cn2cc(Br)cc(Cl)c2n1 ; "B129" ; Cc1cn2cc(-c3ccc4c(=O)n(C5CCN(C(=O)OC(C)(C)C)CC5)cnc4c3)cc(C)c2n1]

Tert-butyl 4-(7-bromo-4-oxoquinazolin-3(4H)-yl)piperidine-1-carboxylate (118 mg, 0.29 mmol), bis(pinacolato)diboron (126 mg, 0.49 mmol), PdCl₂(dppf) (31 mg, 0.043 mmol), and potassium acetate (111 mg, 1.12 mmol) were dissolved in dioxane (2.0 mL). The reaction mixture was bubbled with argon for 10 minutes, heated at 100° C. for 1 hour, then cooled. To the reaction mixture was added 6-bromo-2,8-dimethylimidazo[1,2-a]pyridine (100 mg, 0.45 mmol) in dioxane (1 mL), followed by K₂CO₃ (307 mg, 2.22 mmol) in water (340 μL). The reaction mixture was heated at 100° C. for 2 hours. Upon completion, the reaction mixture was diluted with ethyl acetate (25 mL), and washed with saturated NaHCO₃ (20 mL) and brine (2×20 mL). The organic phase was then filtered under vacuum, dried over Na₂SO₄, filtered, and the filtrated concentrated in vacuo to give a residue. The residue was purified by flash chromatography on a silica gel column using a gradient of 70-100% ethyl acetate in hexanes. Selected fractions were combined and evaporated under reduced pressure to afford tert-butyl 4-(7-(2,8-dimethylimidazo[1,2-a]pyridin-6-yl)-4-oxoquinazolin-3(4H)-yl)piperidine-1-carboxylate (107 mg, 75%) as a solid. LCMS (ES, m/z): 474.3 [M+H]⁺.

### Synthesis of Compound 292

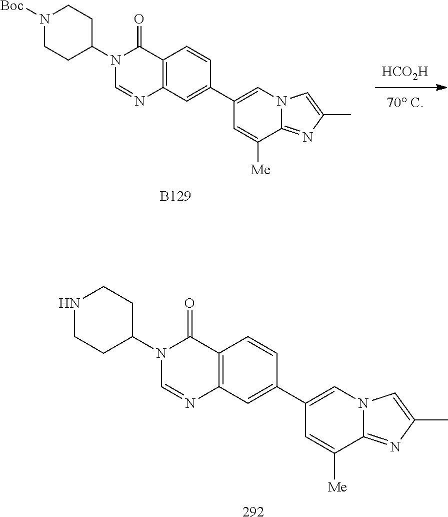 [SMILES: "B129" ; Cc1cn2cc(-c3ccc4c(=O)n(C5CCN(C(=O)OC(C)(C)C)CC5)cnc4c3)cc(C)c2n1 ; Cc1cn2cc(-c3ccc4c(=O)n(C5CCNCC5)cnc4c3)cc(C)c2n1 ; "HCO2H" ; "292"]

A mixture of tert-butyl 4-(7-(2,8-dimethylimidazo[1,2-a]pyridin-6-yl)-4-oxoquinazolin-3(4H)-yl)piperidine-1-carboxylate (102 mg, 0.215 mmol) and formic acid (5 mL) was stirred vigorously at 70° C. for 2 hours. The reaction mixture was concentrated in vacuo to give a residue. The residue was diluted with DCM (20 mL), and washed with 0.5 M NaOH (20 mL) and brine (20 mL). The organic phase was dried over Na₂SO₄, filtered, and the filtrate concentrated under reduced pressure to afford 7-(2,8-dimethylimidazo[1,2-a]pyridin-6-yl)-3-(piperidin-4-yl)quinazolin-4(3H)-one (39 mg, 49%) as a solid. LCMS (ES, m/z): 374.1 [M+H]⁺. ¹H NMR (DMSO-d₆, 300 MHz): δ 8.91 (1H, s), 8.47 (1H, s), 8.21 (1H, d, J=8.4 Hz), 7.97 (1H, d, J=1.8 Hz), 7.88 (1H, dd, J=8.5, 1.9 Hz), 7.70 (1H, s), 7.51 (1H, s), 4.64-4.72 (1H, m), 3.08 (2H, d, J=12.3 Hz), 2.60 (2H, t, J=12.4 Hz), 2.53 (3H, s), 2.35 (3H, s), 1.84-1.98 (2H, m), 1.76 (2H, d, J=12.0 Hz).

### Example 65

### Synthesis of Compound 293

### Synthesis of Intermediate B130

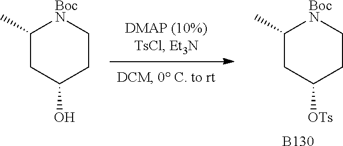 [SMILES: C[C@H]1C[C@@H](O)CCN1C(=O)OC(C)(C)C ; "B130" ; "DMAP (10%)TsCl, Et3N" ; Cc1ccc(S(=O)(=O)O[C@H]2CCN(C(=O)OC(C)(C)C)[C@@H](C)C2)cc1]

A mixture of (2R,4S)-tert-butyl 4-hydroxy-2-methylpiperidine-1-carboxylate (1.00 g, 4.41 mmol), p-toluenesulfonylchloride (1.01 g, 5.30 mmol), and 4-dimethylaminopyridine (53.9 mg, 0.441 mmol) was dissolved in DCM (44 mL) and cooled to 0° C. in an ice bath. To the reaction mixture was added triethylamine (1.8 mL, 13.2 mmol) dropwise. The mixture was warmed to room temperature and stirred for 18 hours, then concentrated under reduced pressure to give a residue. The residue was dissolved in ethyl acetate (70 mL) and washed with saturated NH₄Cl (35 mL), saturated NaHCO₃ (35 mL), and brine (35 mL). The organic phase was dried over Na₂SO₄, filtered, and the filtrate concentrated in vacuo. The residue was purified by flash chromatography on silica gel using a gradient of 0-50% ethyl acetate in hexane. Selected fractions were combined and concentrated under reduced pressure to afford tert-butyl (25,4S)-2-methyl-4-(tosyloxy)piperidine-1-carboxylate (458 mg, 28%) as a solid. LCMS (ES, m/z): 392.1 [M+Na]⁺.

### Synthesis of Intermediate B131

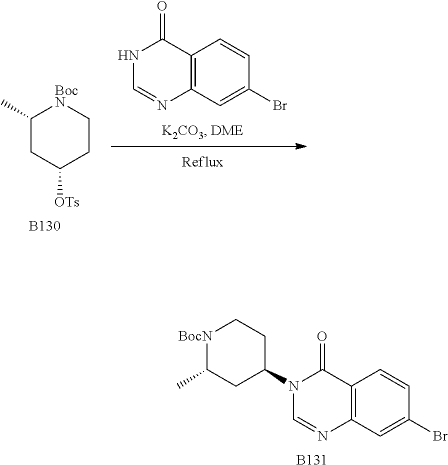

To a mixture of 7-bromoquinazolin-4(3H)-one (135 mg, 0.600 mmol), tert-butyl (2S,4S) methyl-4-(tosyloxy)piperidine-1-carboxylate (443 mg, 1.20 mmol), and potassium carbonate (249 mg, 1.80 mmol) was added 1,2-dimethoxyethane (DME) (3.0 mL). The resulting suspension was stirred at 100° C. for 72 hours. The reaction mixture was diluted with ethyl acetate (20 mL) and filtered through celite. The filter cake was washed with ethyl acetate (15 mL). The filtrate was concentrated under reduced pressure to give a residue and the residue was purified by flash chromatography on silica gel using a gradient of 0-50% ethyl acetate in hexane. Selected fractions were combined and concentrated in vacuo to afford tert-butyl (2R,4R)-4-(7-bromo-4-oxoquinazolin-3(4H)-yl)-2-methylpiperidine-1-carboxylate (92 mg, 36%) as a solid. LCMS (ES, m/z): 422.1 [M+H]⁺.

### Synthesis of Compound 293

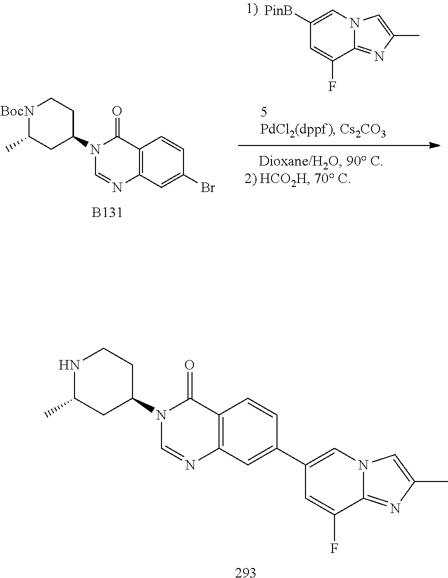

A mixture of tert-butyl (2S,4R)-4-(7-bromo-4-oxoquinazolin-3(4H)-yl)-2-methylpiperidine-1-carboxylate (91 mg, 0.217 mmol), 8-fluoro-2-methyl-6-(4,4,5,5-tetramethyl-1,3,2-dioxaborolan-2-yl)imidazo[1,2-a]pyridine (70 mg, 0.253 mmol), PdCl₂(dppf) (7.9 mg, 11 μmol), and Cs₂CO₃ (71 mg, 0.217 mmol) was dissolved in a mixture of dioxane (2.8 mL) and water (280 μL). The reaction mixture was purged with argon for 10 minutes, then heated at 90° C. for 4 hours. The reaction mixture was diluted with ethyl acetate (40 mL) and washed with saturated NaHCO₃ (25 mL) and brine (2×25 mL). The organic phase was dried over Na₂SO₄, filtered, and the filtrate concentrated under reduced pressure to give a residue. The residue was purified by flash chromatography on a silica gel column using a gradient of 0-5% methanol in DCM. Selected fractions were combined and concentrated in vacuo to afford a solid. To the resulting solid was added neat formic acid (3 mL), and the reaction was stirred vigorously at 70° C. for 2 hours. The reaction mixture was concentrated under reduced pressure and the residue was purified by flash chromatography on a C18 column using a gradient of 5-30% acetonitrile in water with 0.1% formic acid additive. Selected fractions were combined, neutralized with (NH₄)₂CO_{3,} and lyophilized. The resulting solid was suspended in DCM (10 mL) and 0.2 N NaOH (10 mL), extracted, and the phases were separated. The aqueous layer was extracted with DCM (2×10 mL). The combined organic layers were dried over Na₂SO₄, filtered, and the filtrate concentrated in vacuo to afford 7-(8-fluoro-2-methylimidazo[1,2-c]pyridin-6-yl)-3-((2S,4R)-2-methylpiperidin-4-yl)quinazolin-4(3H)-one (49 mg, 64%) as a solid. LCMS (ES, m/z): 392.2 [M+H]⁺. ¹H NMR (CDCl₃, 300 MHz): δ 8.39 (1H, d,=8.3 Hz), 8.23 (1H, s), 8.21 (1H, d,=1.5 Hz), 7.86 (1H, d, J=1.8 Hz), 7.67 (1H, dd, J=8.3, 1.8 Hz), 7.50 (1H, d, J=2.8 Hz), 7.20 (1H, dd, J=11.1, 1.4 Hz), 5.23-5.31 (1H, m), 3.67 (1H, s), 3.14-3.26 (2H, m), 2.51 (3H, s), 2.16-2.24 (1H, m), 1.96-2.06 (2H, m), 1.91 (1H, d, J=13.1 Hz), 1.42 (3H, d, J=6.9 Hz).

### Example 66

### Synthesis of Compound 294

### Synthesis of Intermediate B132

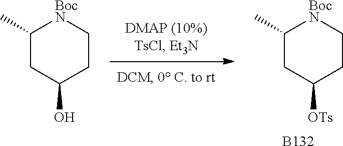 [SMILES: "B132" ; C[C@H]1C[C@H](O)CCN1C(=O)OC(C)(C)C ; "DMAP (10%)TsCl, Et3N" ; Cc1ccc(S(=O)(=O)O[C@@H]2CCN(C(=O)OC(C)(C)C)[C@@H](C)C2)cc1]

A mixture of (2S,4R)-tert-butyl 4-hydroxy-2-methylpiperidine-1-carboxylate (250 mg, 1.10 mmol), p-toluenesulfonylchloride (252 mg, 1.32 mmol), and 4-dimethylaminopyridine (13 mg, 0.110 mmol) was dissolved in DCM (11 mL) and cooled to 0° C. in an ice bath. To this mixture was added triethylamine (460 μL, 3.31 mmol) dropwise. The reaction mixture was warmed to room temperature and stirred for 18 hours, then concentrated under reduced pressure to give a residue. The residue was dissolved in ethyl acetate (70 mL) and washed with saturated NH₄Cl (35 mL), saturated NaHCO₃ (35 mL), and brine (35 mL). The organic phase was dried over Na₂SO₄, filtered, and the filtrate concentrated in vacuo to give a residue. The residue was purified by flash chromatography on silica gel using a gradient of 0-50% ethyl acetate in hexanes. Selected fractions were combined and concentrated under reduced pressure to afford tert-butyl (25,4R)-2-methyl-4-(tosyloxy)piperidine-1-carboxylate (179 mg, 44%) as a solid. LCMS (ES, m/z): 392.1 [M+Na]⁺.

### Synthesis of Intermediate B133

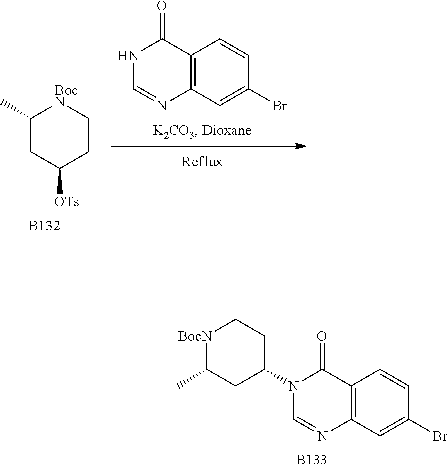

To a mixture of 7-bromoquinazolin-4(3H)-one (80 mg, 0.355 mmol), tert-butyl (2S,4R)-2-methyl-4-(tosyloxy)piperidine-1-carboxylate (263 mg, 0.711 mmol), and potassium carbonate (149 mg, 1.07 mmol) was added 1,4-dioxane (1.5 mL). The resulting suspension was stirred under reflux for 72 hours. The reaction mixture was diluted with ethyl acetate (20 mL), and filtered through celite. The filter cake was washed with ethyl acetate (15 mL). The filtrate was concentrated under reduced pressure, and the residue purified by flash chromatography on silica gel using a gradient of 0-50% ethyl acetate in hexane. Selected fractions were combined and concentrated in vacuo to afford tert-butyl (2S,4S)-4-(7-bromo-4-oxoquinazolin-3 (4H)-yl)-2-methylpiperidine-1-carboxylate (64 mg, 43%) as a solid. LCMS (ES, m/z): 422.1 [M+H]⁺.

### Synthesis of Compound 294

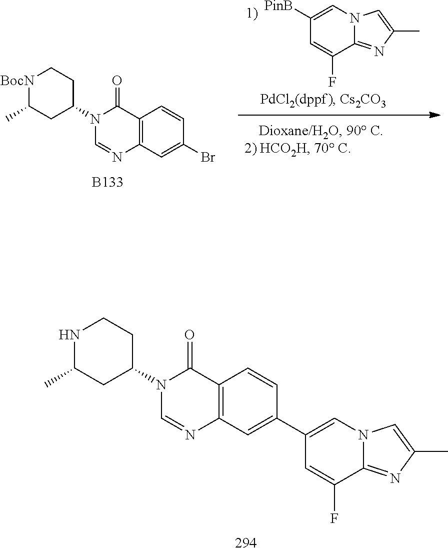

A mixture of tert-butyl (2S,4S)-4-(7-bromo-4-oxoquinazolin-3(4H)-yl)-2-methylpiperidine-1-carboxylate 4 (64 mg, 0.152 mmol), 8-fluoro-2-methyl-6-(4,4,5,5-tetramethyl-1,3,2-dioxaborolan-2-yl)imidazo[1,2-a]pyridine 5 (49 mg, 0.177 mmol), PdCl₂(dppf) (6.0 mg, 7.6 μmol), and Cs₂CO₃ (148 mg, 0.455 mmol) was dissolved in dioxane (2.8 mL) and water (280 μL). The reaction mixture was purged with argon for 10 minutes, then heated at 90° C. for 4 hours. The reaction mixture was diluted with ethyl acetate (40 mL) and washed with saturated NaHCO₃ (25 mL) and brine (2×25 mL). The organic phase was filtered under vacuum, dried over Na₂SO₄, and concentrated under reduced pressure to give a residue. The residue was purified by flash chromatography on a silica gel column using a gradient of 80-100% ethyl acetate in hexane. Selected fractions were combined and evaporated in vacuo. To the resulting solid was added neat formic acid (3 mL) and the reaction mixture was stirred vigorously at 70° C. for 2 hours, then concentrated under reduced pressure to give a residue. The residue was purified by flash chromatography on a C18 column using a gradient of 5-30% MeCN in water with 0.1% formic acid additive. Selected fractions were combined, neutralized with (NH₄)₂CO_{3,} and lyophilized. The resulting solid was suspended in DCM (10 mL) and 0.2 N NaOH (10 mL), extracted, and the phases were separated. The aqueous layer was further extracted with DCM (2×10 mL). The combined organic layers were dried over Na₂SO₄, filtered, and the filtrate concentrated in vacuo to afford 7-(8-fluoro-2-methylimidazo[1,2-a]pyridin-6-yl)-3-((2S,4S)-2-methylpiperidin-4-yl)quinazolin-4(3H)-one (24 mg, 54%) as a solid. LCMS (ES, m/z): 392.2 [M+H]⁺. ¹H NMR (CDCl₃, 300 MHz): δ 8.41 (1H, d, J=8.3 Hz), 8.23 (2H, m), 7.88 (1H, d, J=1.8 Hz), 7.69 (1H, dd, J=8.3, 1.9 Hz), 7.52 (1H, d, J=3.0 Hz), 7.22 (1H, dd, J=11.1, 1.4 Hz), 4.98-5.12 (1H, m), 3.35-3.41 (1H, m), 2.93-3.04 (2H, m), 1.93-2.08 (3H, m), 1.59-1.72 (1H, m), 1.28 (3H, d, J=6.2 Hz).

### Example 67

### Synthesis of Compound 295

### Synthesis of Intermediate B134

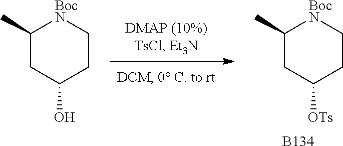 [SMILES: C[C@@H]1C[C@@H](O)CCN1C(=O)OC(C)(C)C ; "B134" ; "DMAP (10%)TsCl, Et3N" ; Cc1ccc(S(=O)(=O)O[C@H]2CCN(C(=O)OC(C)(C)C)[C@H](C)C2)cc1]

A mixture of (2R,4S)-tert-butyl 4-hydroxy-2-methylpiperidine-1-carboxylate (1.00 g, 4.41 mmol), p-toluenesulfonylchloride (1.01 g, 5.30 mmol), and 4-dimethylaminopyridine (53.9 mg, 0.441 mmol) was dissolved in DCM (44 mL) and cooled to 0° C. in an ice bath. To this mixture was added triethylamine (1.8 mL, 13.2 mmol) dropwise. The reaction mixture was warmed to room temperature and stirred for 18 hours, then concentrated under reduced pressure to give a residue. The residue was dissolved in ethyl acetate (70 mL) and washed with saturated NH₄Cl (35 mL), saturated NaHCO₃ (35 mL), and brine (35 mL). The organic phase was dried over Na₂SO₄, filtered, and the filtrate concentrated in vacuo to give a residue. The residue was purified by flash chromatography on silica gel using a gradient of 0-50% ethyl acetate in hexane. Selected fractions were combined and concentrated under reduced pressure to afford tert-butyl (2R,4S)-2-methyl (tosyloxy)piperidine-1-carboxylate (1.14 g, 70%) as a solid. LCMS (ES, m/z): 392.2 [M+Na]⁺.

### Synthesis of Intermediate B135

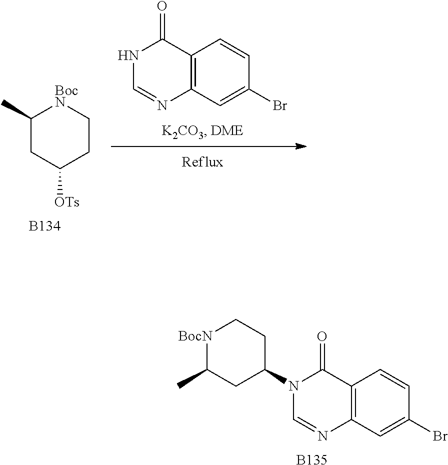

To a mixture of 7-bromoquinazolin-4(3H)-one (320 mg, 1.42 mmol), tert-butyl (2R,4S)-2-methyl-4-(tosyloxy)piperidine-1-carboxylate (1.05 g, 2.84 mmol), and potassium carbonate (590 mg, 4.27 mmol) was added 1,2-dimethoxyethane (DME) (7.11 mL). The resulting suspension was stirred at 100° C. for 72 hours, then diluted with ethyl acetate (20 mL) and filtered through celite. The filter cake was washed with ethyl acetate (15 mL). The filtrate was concentrated under reduced pressure to give a residue and the residue was purified by flash chromatography on silica gel using a gradient of 0-50% ethyl acetate in hexane. Selected fractions were combined and concentrated in vacuo to afford tert-butyl (2R,4R)-4-(7-bromo-4-oxoquinazolin-3(4H)-yl)-2-methylpiperidine-1-carboxylate (349 mg, 58%) as a solid. LCMS (ES, m/z): 422.1 [M+H]⁺.

### Synthesis of Compound 295

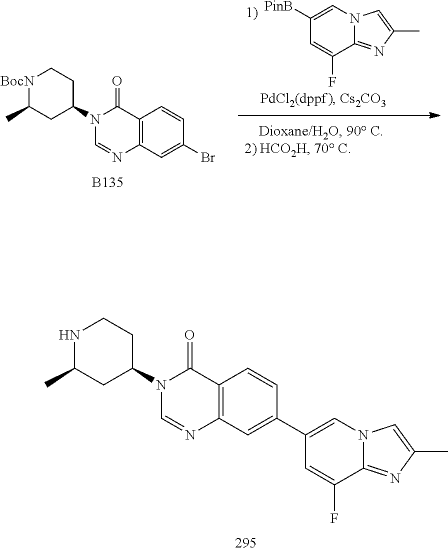

A mixture of tert-butyl (2R,4R)-4-(7-bromo-4-oxoquinazolin-3(4H)-yl)-2-methylpiperidine-1-carboxylate (120 mg, 0.284 mmol), 8-fluoro-2-methyl-6-(4,4,5,5-tetramethyl-1,3,2-dioxaborolan-2-yl)imidazo[1,2-a]pyridine (92 mg, 0.33 mmol), PdCl₂(dppf) (21 mg, 0.028 mmol), and Cs₂CO₃ (278 mg, 0.852 mmol) was dissolved in dioxane (3.4 mL) and water (340 μL). The reaction mixture was bubbled with argon for 10 minutes, then heated at 90° C. for 4 hours. The reaction mixture was diluted with ethyl acetate (40 mL) and washed with saturated NaHCO₃ (25 mL) and brine (2×25 mL). The organic phase was dried over Na₂SO₄, filtered, and the filtrate concentrated under reduced pressure to give a residue. The residue was purified by flash chromatography on a silica gel column using a gradient of 80-100% ethyl acetate in hexanes. Selected fractions were combined and evaporated in vacuo to afford a solid. To the resulting solid was added neat formic acid (5 mL) and stirred vigorously at 70° C. for 2 hours. The reaction mixture was concentrated under reduced pressure to give a residue and the residue was purified by flash chromatography on a C18 column using a gradient of 5-30% MeCN in water with 0.1% formic acid additive. Selected fractions were combined, neutralized with (NH₄)₂CO_{3,} and lyophilized. The resulting solid was suspended in DCM (10 mL) and 0.2 N NaOH (10 mL), extracted, and the phases were separated. The aqueous layer was extracted with DCM (2×10 mL). The combined organic layers were dried over Na₂SO₄, filtered, and the filtrate concentrated in vacuo to afford 7-(8-fluoro-2-methylimidazo[1,2-c]pyridin-6-yl)-3-((2R,4R)-2-methylpiperidin-4-yl)quinazolin-4(3H)-one (25 mg, 22%) as a solid. LCMS (ES, m/z): 392.2 [M+H]⁺. ¹H NMR (CDCl₃, 300 MHz): δ 8.41 (1H, d, J=8.3 Hz), 8.23 (1H, d, J=1.5 Hz), 8.22 (1H, s), 7.88 (1H, d, J=1.8 Hz), 7.69 (1H, dd, J=8.3, 1.9 Hz), 7.52 (1H, dd, J=3.0, 1.0 Hz), 7.22 (1H, dd, J=11.1, 1.5 Hz), 4.96-5.07 (1H, m), 3.35 (1H, d, J=12.6 Hz), 2.92-3.01 (2H, m), 2.54 (3H, s), 2.05 (2H, d, J=13.4 Hz), 1.85-1.99 (1H, m), 1.54-1.66 (1H, m), 1.25 (3H, d, J=6.3 Hz).

### Example 68

### Synthesis of Compound 296

### Synthesis of Intermediate B136

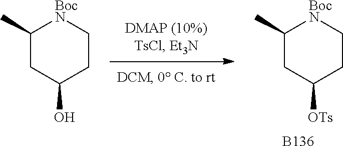 [SMILES: "B136" ; C[C@@H]1C[C@H](O)CCN1C(=O)OC(C)(C)C ; "DMAP (10%)TsCl, Et3N" ; Cc1ccc(S(=O)(=O)O[C@@H]2CCN(C(=O)OC(C)(C)C)[C@H](C)C2)cc1]

A mixture of (2R,4R)-tert-butyl 4-hydroxy-2-methylpiperidine-1-carboxylate (1.00 g, 4.41 mmol), p-toluenesulfonylchloride (1.01 g, 5.30 mmol), and 4-dimethylaminopyridine (53.9 mg, 0.441 mmol) was dissolved in DCM (44 mL), then cooled to 0° C. in an ice bath. To this mixture was added triethylamine (1.8 mL, 13.2 mmol) dropwise. The reaction mixture was warmed to room temperature and stirred for 18 hours, then concentrated under reduced pressure to give a residue. The residue was dissolved in ethyl acetate (70 mL) and washed with saturated NH₄Cl (35 mL), saturated NaHCO₃ (35 mL), and brine (35 mL). The organic phase was dried over Na₂SO₄, filtered, and the filtrate concentrated in vacuo to give a residue. The residue was purified by flash chromatography on silica gel using a gradient of 0-50% ethyl acetate in hexane. Selected fractions were combined and concentrated under reduced pressure to afford tert-butyl (2R,4R)-2-methyl (tosyloxy)piperidine-1-carboxylate (322 mg, 20%) as a solid. LCMS (ES, m/z): 392.1 [M+Na]⁺.

### Synthesis of Intermediate B137

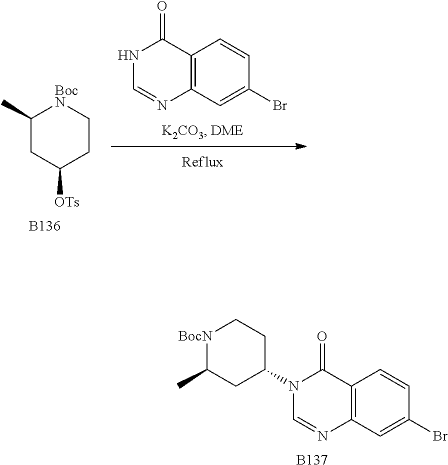

To a mixture of 7-bromoquinazolin-4(3H)-one (98 mg, 0.436 mmol), tert-butyl (2S,4S)-2-methyl-4-(tosyloxy)piperidine-1-carboxylate (322 mg, 0.872 mmol), and potassium carbonate (181 mg, 1.31 mmol) was added 1,2-dimethoxyethane (DME) (2.2 mL). The resulting suspension was stirred at 100° C. for 72 hours, then diluted with ethyl acetate (20 mL) and filtered through celite. The filter cake was washed with ethyl acetate (15 mL). The filtrate was concentrated under reduced pressure to give a residue, and the residue was purified by flash chromatography on silica gel using a gradient of 0-50% ethyl acetate in hexane. Selected fractions were combined and concentrated in vacuo to afford tert-butyl (2R,4S)-4-(7-bromo-4-oxoquinazolin-3(4H)-yl)-2-methylpiperidine-1-carboxylate (105 mg, 57%) as a solid. LCMS (ES, m/z): 422.1 [M+H]⁺.

### Synthesis of Compound 296

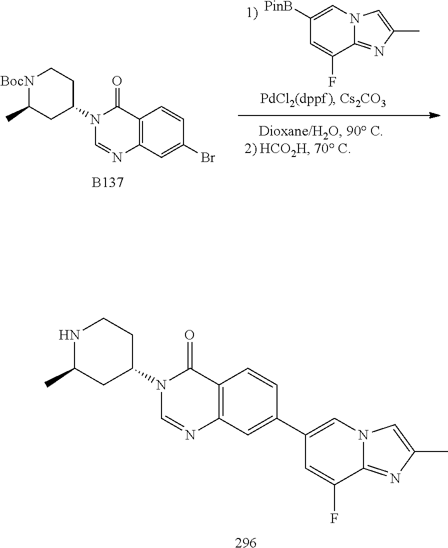

A mixture of tert-butyl (25,4R)-4-(7-bromo-4-oxoquinazolin-3(4H)-yl)-2-methylpiperidine-1-carboxylate (76 mg, 0.181 mmol), 8-fluoro-2-methyl-6-(4,4,5,5-tetramethyl-1,3,2-dioxaborolan-2-yl)imidazo[1,2-a]pyridine (58 mg, 0.212 mmol), PdCl₂(dppf) (6.6 mg, 9.0 μmol), and Cs₂CO₃ (177 mg, 0.543 mmol) was dissolved in dioxane (2.8 mL) and water (280 μL). The reaction mixture was purged with argon for 10 minutes, then heated at 90° C. for 4 hours. The reaction mixture was diluted with ethyl acetate (40 mL) and washed with saturated NaHCO₃ (25 mL) and brine (2×25 mL). The organic phase was dried over Na₂SO₄, filtered, and the filtrate concentrated under reduced pressure to give a residue. The residue was purified by flash chromatography on a silica gel column using a gradient of 0-10% methanol in DCM. Selected fractions were combined and evaporated in vacuo to yield a solid. To the resulting solid was added neat formic acid (3 mL) and the reaction mixture was stirred vigorously at 70° C. for 2 hours, then concentrated under reduced pressure to give a residue. The residue was purified by flash chromatography on a C18 column using a gradient of 5-30% acetonitrile in water with 0.1% formic acid additive. Selected fractions were combined, neutralized with (NH₄)₂CO_{3,} and lyophilized. The resulting solid was suspended in DCM (10 mL) and 0.2 N NaOH (10 mL), extracted, and the phases were separated. The aqueous layer was extracted with DCM (2×10 mL). The combined organic layers were dried over Na₂SO₄, filtered, and the filtrate concentrated in vacuo to afford 7-(8-fluoro-2-methylimidazo[1,2-a]pyridin-6-yl)-3-((2R,4S)-2-methylpiperidin-4-yl)quinazolin-4(3H)-one (26 mg, 36%) as a solid. LCMS (ES, m/z): 392.1 [M+H]⁺. ¹H NMR (CDCl₃, 300 MHz): δ 8.39 (1H, d, J=8.3 Hz), 8.23 (1H, s), 8.20 (1H, d, J=1.4 Hz), 7.85 (1H, d, J=1.8 Hz), 7.67 (1H, dd, J=8.3, 1.8 Hz), 7.50 (1H, d, J=2.9 Hz), 7.20 (1H, dd, J=11.1, 1.4 Hz), 5.21-5.29 (1H, m), 3.55-3.58 (1H, m), 3.15-3.22 (1H, m), 3.05 (1H, dt, J=12.8, 3.8 Hz), 2.51 (3H, s), 2.03-2.13 (2H, m), 1.86-1.93 (2H, m), 1.36 (3H, d, J=6.9 Hz).

### Example 69

### Synthesis of Compounds 304 and 305

### Synthesis of Intermediate B138

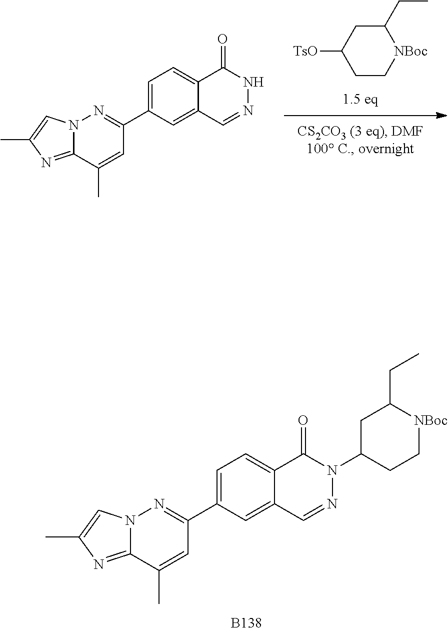 [SMILES: Cc1cn2nc(-c3ccc4c(=O)[nH]ncc4c3)cc(C)c2n1 ; CCC1CC(OS(=O)(=O)c2ccc(C)cc2)CCN1C(=O)OC(C)(C)C ; "B138" ; CCC1CC(n2ncc3cc(-c4cc(C)c5nc(C)cn5n4)ccc3c2=O)CCN1C(=O)OC(C)(C)C]

A mixture of 6-{2,8-dimethylimidazo[1,2-b]pyridazin-6-yl}-2H-phthalazin-1-one (80.0 mg, 0.27 mmol, 1.00 equiv), tert-butyl 2-ethyl-4-[(4-methylbenzenesulfonyl)oxy]piperidine-1-carboxyl ate (157.9 mg, 0.41 mmol, 1.5 equiv) and Cs₂CO₃ (268.4 mg, 0.82 mmol, 3 equiv) in DMF (1.6 mL) was stirred overnight at 100° C. The resulting mixture was diluted with water (10 mL), then extracted with ethyl acetate (3×10 mL). The combined organic layers were washed with brine (3×10 mL), dried over anhydrous Na₂SO₄, and filtered. After filtration, the filtrate was concentrated under reduced pressure to give a residue. The residue was purified by silica gel column chromatography, eluted with PE/EA (0:1) to afford tert-butyl 446-{2,8-dimethylimidazo[1,2-b]pyridazin-6-yl}-1-oxophthalazin-2-yl)-2-ethylpiperidine-1-carboxylate (65 mg, 47.09%) as a solid. LCMS (ES, m/z): 503 [M+H]⁺.

### Synthesis of Compounds 304 and 305

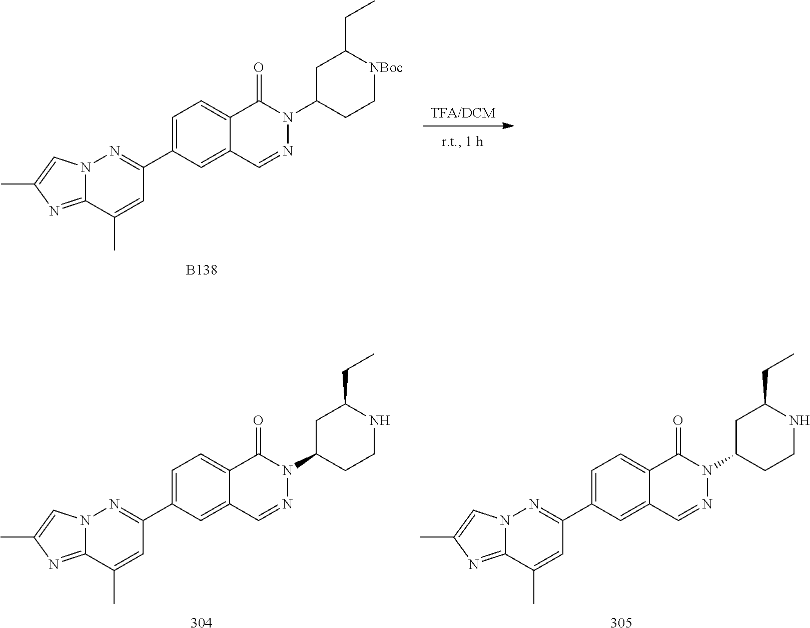

A mixture of tert-butyl 4-(6-{2,8-dimethylimidazo[1,2-b]pyridazin-6-yl}-1-oxophthalazin-2-yl)-2-ethylpiperidine-1-carboxylate (65 mg, 0.13 mmol, 1.00 equiv), TFA (0.5 mL) and DCM (2 mL) was stirred for 1 h at room temperature. The resulting mixture was concentrated under vacuum to give a residue. The residue was purified by CHIRAL-HPLC (Column: CHIRALPAK IG, 2*25 cm, 5 um; Mobile Phase A: MtBE (0.1% DEA)-HPLC, Mobile Phase B: EtOH; Flow rate: 20 mL/min; Gradient: 25% B to 25% B in 13 min) to afford 6-{2,8-dimethylimidazo[1,2-b]pyridazin-6-yl}-2-[(2R,4R)-2-ethylpiperidin-4-yl]phthalazin-1-one (5.2 mg, 9.95%) and 6-{2,8-dimethylimidazo[1,2-b]pyridazin-6-yl}-2-[(2R,4S)-2-ethylpiperidin-4-yl]phthalazin-1-one (1.6 mg, 2.96%) as solids. Compound 304: LCMS (ES, m/z): 403 [M+H]⁺. ¹H NMR (400 MHz, DMSO-d₆) δ 8.62 — 8.55 (m, 2H), 8.48 (dd, J=8.4, 1.7 Hz, 1H), 8.38 (d, J=8.4 Hz, 1H), 8.11 (s, 1H), 7.78 (s, 1H), 4.98 (tt, J=11.8, 4.1 Hz, 1H), 3.10 (dt, J=12.4, 3.3 Hz, 1H), 2.74-2.63 (m, 1H), 2.64 (s, 3H), 2.50 (s, 1H)_{2.43} (s, 3H), 1.90 — 1.67 (m, 3H), 1.51 (q, J=11.7 Hz, 1H), 1.38 (ddp, J=20.8, 14.0, 6.9 Hz, 2H), 0.89 (t, J=7.4 Hz, 3H). Compound 305: LCMS (ES, m/z): 403 [M+H]⁺. ¹H NMR (400 MHz, DMSO-d₆) δ 8.63 (d, J=12.0 Hz, 2H), 8.51 (dd, J=8.5, 1.7 Hz, 1H), 8.40 (d, J=8.4 Hz, 1H), 8.13 (s, 1H), 7.80 (s, 1H), 5.25 (tt, J=8.3, 4.4 Hz, 1H), 3.30 (s, 2H), 3.26 (s, 1H), 2.65 (s, 3H), 2.44 (s, 3H), 2.19 (dtd, J=18.0, 8.4, 3.7 Hz, 2H), 2.05-1.96 (m, 1H), 1.91 (dt, J=13.7, 5.3 Hz, 1H), 1.72 (p, J=7.9, 7.4 Hz, 2H), 0.96 (t, J=7.4 Hz, 3H).

### Example 70

### Synthesis of Compound 309

### Synthesis of Intermediate B139

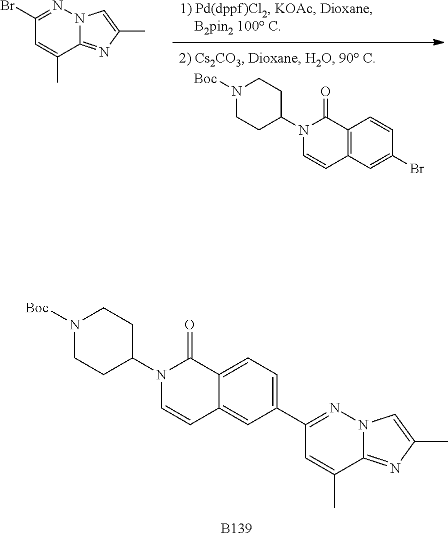 [SMILES: Cc1cn2nc(Br)cc(C)c2n1 ; CC(C)(C)OC(=O)N1CCC(n2ccc3cc(Br)ccc3c2=O)CC1 ; "B139" ; Cc1cn2nc(-c3ccc4c(=O)n(C5CCN(C(=O)OC(C)(C)C)CC5)ccc4c3)cc(C)c2n1]

A mixture of 6-bromo-2,8-dimethylimidazo[1,2-b]pyridazine (188 mg, 0.83 mmol), Bis(pinacolato)diboron (211 mg, 0.83 mmol), Pd(dppf)Cl₂ (47 mg, 0.06 mmol), and potassium acetate (188 mg, 1.92 mmol) in dioxane (4.3 mL) was heated to 100° C. for 1.5 h. To the reaction mixture was added a solution of tert-butyl 4-(6-bromo-1-oxoisoquinolin-2(1H)-yl)piperidine-1-carboxylate (260 mg, 0.64 mmol) in dioxane (3.5 mL), followed by cesium carbonate (624 mg, 1.92 mmol) and water (0.9 mL) under argon. This resulting mixture was heated at 90° C. for 2 h, cooled to room temperature and filtered through celite using 20% methanol in DCM as eluent. The volatiles were evaporated under reduced pressure. Water (20 mL) and DCM (20 mL) were added, and the layers were separated. The aqueous layer was extracted with DCM (3×20 mL). The organic layers were combined, dried over Na₂SO₄, filtered, and the filtrate concentrated under reduced pressure to give a residue. The residue was purified by flash chromatography on silica gel using a gradient of 0-10% methanol in DCM to afford tert-butyl 4-(6-(2,8-dimethylimidazo[1,2-b]pyridazin-6-yl)-1-oxoisoquinolin-2(1H)-yl)piperidine-1-carboxylate (267 mg, 88%) as a solid. LCMS (ES, m/z): 474.2 [M+H]⁺.

### Synthesis of Compound 309

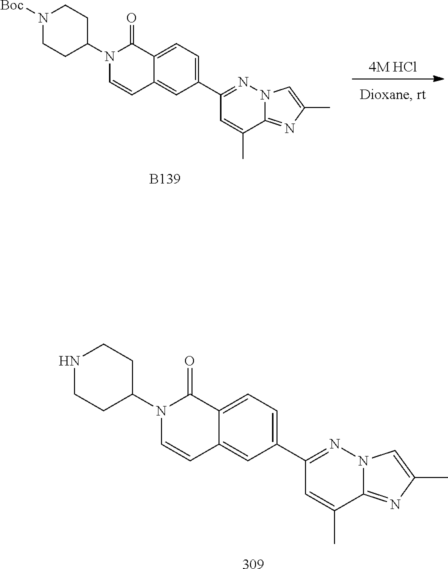 [SMILES: "B139" ; Cc1cn2nc(-c3ccc4c(=O)n(C5CCN(C(=O)OC(C)(C)C)CC5)ccc4c3)cc(C)c2n1 ; Cc1cn2nc(-c3ccc4c(=O)n(C5CCNCC5)ccc4c3)cc(C)c2n1 ; "4M HCl" ; "309"]

To a solution of tert-butyl 4-(6-(2,8-dimethylimidazo[1,2-b]pyridazin-6-yl)-1-oxoisoquinolin-2(1H)-yl)piperidine-1-carboxylate (267 mg, 564 umol) in dioxane (11.3 mL) was added 4.0 M HCl in dioxane (11.3 mL, 45.1 mmol). The resulting mixture was stirred at room temperature for 1 hour. The reaction mixture was concentrated in vacuo, taken up in CH₂Cl₂ (30 mL), and washed with saturated NaHCO₃ (15 mL). The aqueous phase was extracted with DCM (2×20 mL). The organic layers were combined, dried over Na₂SO₄, filtered, and the filtrate concentrated in vacuo to give a residue. The residue was purified on a silica gel cartridge using a gradient of MeOH/NH₄OH (9:1) from 0-20% in CH₂Cl₂ to afford 6-(2,8-dimethylimidazo[1,2-b]pyridazin-6-yl)-2-(piperidin-4-yl)isoquinolin-1(2H)-one (167 mg, 79%) as a solid. LCMS (ES, m/z): 374.1 [M+H]⁺. ¹H NMR (CHCl₃-d, 400 MHz): δ_{H} 8.55 (1H, d, J=8.4 Hz), 8.06-8.03 (2H, m), 7.80 (1H, s), 7.34 (1H, s), 7.24 (1H, s), 6.64 (1H, d, J=7.5 Hz), 5.12-5.18 (1H, m), 3.25 (2H, d, J=12.2 Hz), 2.88 (2H, t, J=11.9 Hz), 2.74 (3H, s), 2.55 (3H, s), 1.95 (2H, d, J=11.9 Hz), 1.83-1.74 (2H, m).

### Example 71

### Synthesis of Compound 310

### Synthesis of Compound 310

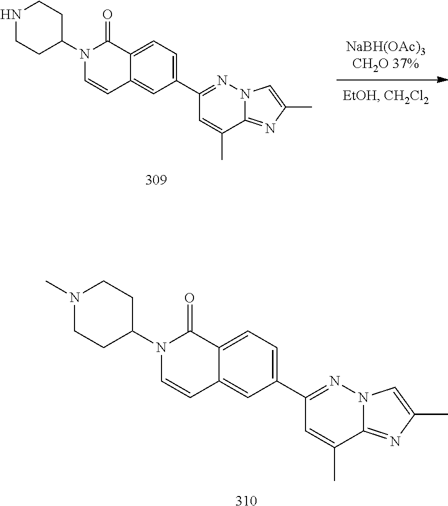

To a solution of 6-(2,8-dimethylimidazo[1,2-b]pyridazin-6-yl)-2-(piperidin-4-yl)isoquinolin-1(2H)-one (90.0 mg, 229 umol), CH₂Cl₂ (1.0 mL) in ethanol (0.2 mL) was added a solution of formaldehyde 37% in water (85.2 uL, 1.14 mmol). The mixture was stirred at room temperature for 1 hour. Then NaBH(OAc)₃ (291 mg, 1.37 mmol) was added and reaction mixture was stirred for an additional 2 hours at room temperature. The reaction mixture was concentrated under reduced pressure, then diluted with CH₂Cl₂ (30 mL) and washed with saturated aqueous NaHCO₃ (10 mL). The organic layer was dried over Na₂SO₄ and the solvent was removed in vacuo to give a residue. The residue was purified by flash chromatography on a silica gel column using a gradient of 0 to 20% methanol in CH₂Cl₂ to afford 6-(2,8-dimethylimidazo[1,2-b]pyridazin-6-yl)-2-(1-methylpiperidin-4-yl)isoquinolin-1(2H)-one (63.0 mg, 71%) as a solid. LCMS (ES, m/z): 388.2 [M+H]⁺. ¹H NMR (CHCl₃-d, 400 MHz): δ_{H} 8.55 (1H, d, J=8.3 Hz), 8.04 (2H, d, J=10.9 Hz), 7.80 (1H, s), 7.33 (1H, s), 7.23 (1H, d, J=7.6 Hz), 6.64 (1H, d, J=7.5 Hz), 5.07 (1H, p, J=8.1 Hz), 3.02 (2H, d, J=11.4 Hz), 2.74 (3H, s), 2.55 (3H, s), 2.36 (3H, s), 2.24 (2H, t, J=8.7 Hz), 1.94 (4H, s).

### Example 72

### Synthesis of Compound 311

### Synthesis of Intermediate B140

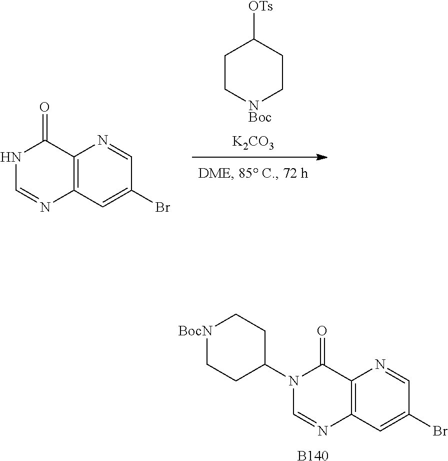

A mixture of 7-bromopyrido[3,2-d]pyrimidin-4(3H)-one (0.25 g, 1.1 mmol), tert-butyl 4-(tosyloxy)piperidine-1-carboxylate (1.2 g, 3.3 mmol), and K₂CO₃ (0.31 g, 2.2 mmol) in DME (8.0 mL) was heated to 85° C. for 72 h and then cooled to room temperature. The reaction mixture was filtered, and the volatiles were evaporated under reduced pressure. Water (20 mL) and DCM (20 mL) were added, and the layers were separated. The aqueous layer was extracted with DCM (3×20 mL). The organic layers were combined, dried over Na₂SO₄, filtered, and the filtrate concentrated under reduced pressure to give a residue. The residue was purified by column chromatography on silica gel using a gradient of 0-100% ethyl acetate in hexane to afford tert-butyl 4-(7-bromo-4-oxopyrido[3,2-d]pyrimidin-3(4H) yl)piperidine-1-carboxylate (0.11 g, 24%) as a solid. LCMS (ES, m/z): 431.1 [M+Na]⁺.

### Synthesis of Intermediate B141

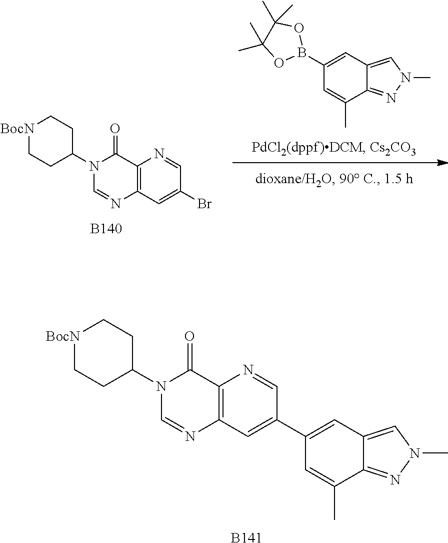

A mixture of tert-butyl 4-(7-bromo-4-oxopyrido[3,2-d]pyrimidin-3(4H) yl)piperidine-1-carboxylate (108 mg, 0.26 mmol), 2,7-dimethyl-5-(4,4,5,5-tetramethyl-1,3,2-dioxaborolan-2-yl)-2H-indazole (108 mg, 0.396 mmol), Cs₂CO₃ (215 mg, 0.66 mmol), and Pd(dppf)Cl₂.DCM (22 mg, 0.0264 mmol) in a mixture of dioxane (4.0 mL) and water (0.5 mL) was heated to 90° C. for 1.5 h and then cooled to room temperature. The reaction mixture was filtered over Celite using 10% methanol in DCM as eluent. The volatiles were evaporated under reduced pressure. The crude material was purified by column chromatography on silica gel using a gradient of 0-10% methanol in ethyl acetate to afford tert-butyl 4-(7-(2,7-dimethyl-2H-indazol-5-yl)-4-oxopyrido[3,2-d]pyrimidin-3(4H)-yl)piperidine-1-carboxylate (101 mg, 81%) as a solid. LCMS (ES, m/z): 475.2 [M+H]⁺.

### Synthesis of Compound 311

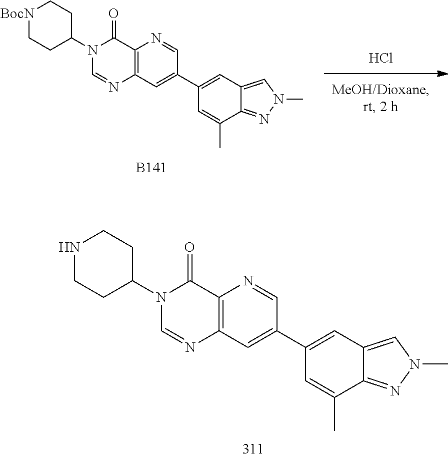 [SMILES: "B141" ; Cc1cc(-c2cnc3c(=O)n(C4CCN(C(=O)OC(C)(C)C)CC4)cnc3c2)cc2cn(C)nc12 ; Cc1cc(-c2cnc3c(=O)n(C4CCNCC4)cnc3c2)cc2cn(C)nc12 ; "311" ; Cl]

To a solution of tert-butyl 4-(7-(2,7-dimethyl-2H-indazol-5-yl)-4-oxopyrido[3,2-d]pyrimidin-3(4H)-yl)piperidine-1-carboxylate (101 mg, 0.21 mmol) in methanol (8.0 mL) was added 4 M HCl solution in dioxane (7.0 mL, 28 mmol). The reaction mixture was stirred at room temperature for 2 h. The volatiles were evaporated under reduced pressure. An aqueous solution of NaHCO₃ (20 mL) and DCM (30 mL) were added, and the layers were separated. The aqueous layer was extracted with DCM (3×30 mL). The organic layers were combined, dried over sodium sulfate, filtered, and the filtrate concentrated under reduced pressure to give a residue. The residue was purified by column chromatography on silica gel using a gradient of 0-20% MeOH:Et3N (2:1 ratio) in DCM. The fractions containing product were collected and evaporated under reduced pressure. Water (10 mL) and DCM (10 mL) were added, and the layers were separated. The aqueous layer was extracted with DCM (3×10 mL). The organic layers were combined, dried over sodium sulfate, filtered, and the filtrate concentrated under reduced pressure to afford 7-(2,7-dimethyl-2H-indazol-5-yl)-3-(piperidin-4-yl)pyrido[3,2-d]pyrimidin-4(3H)-one (48 mg, 60%) as a solid. LCMS (ES, m/z): 375.2 [M+H]⁺. ¹H NMR (DMSO-d₆, 400 MHz): δ_{H} 9.20 (1H, d, J=2.2 Hz), 8.53 (1H, s), 8.46 (1H, s), 8.31 (1H, d, J=2.2 Hz), 8.12 (1H, s), 7.56 (1H, s), 4.72 (1H, t, J=11.8 Hz), 4.22 (3H, s), 3.10 (2H, d, J=12.2 Hz), 2.60-2.66 (5H, m), 1.80-1.98 (4H, m).

### Example 73

### Synthesis of Compound 312

### Synthesis of Intermediate B142

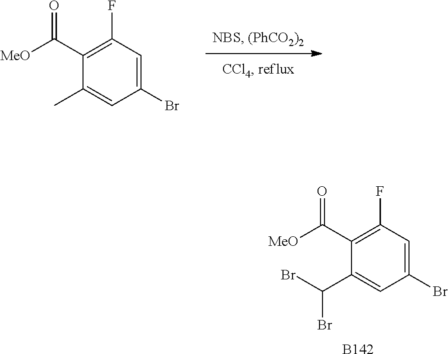

A methyl 4-bromo-2-fluoro-6-methylbenzoate (1.00 g, 3.97 mmol) andN-bromosuccinimide (1.57 g, 8.73 mmol) was dissolved in CCl₄ (26 mL). To the reaction mixture was added benzoyl peroxide (64.1 mg, 0.198 mmol). The reaction mixture was heated under reflux overnight, then cooled to room temperature and diluted with DCM (100 mL). The organic phase was washed with 1 M Na₂S₂O₃ (50 mL), saturated NaHCO₃ (50 mL), and brine (2×50 mL). The organic phase was dried over Na₂SO₄, filtered, and the filtrate concentrated under reduced pressure. The residue was purified by flash chromatography on silica gel using a gradient of 0-20% ethyl acetate in hexane to afford methyl 4-bromo-2-(dibromomethyl)-6-fluorobenzoate (860 mg, 54%) as an oil. LCMS (ES, m/z): 404.7 [M+H]⁺.

### Synthesis of Intermediate B143

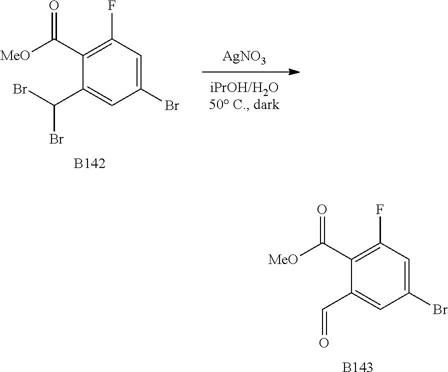

To a solution of methyl 4-bromo-2-(dibromomethyl)-6-fluorobenzoate (360 mg, 0.889 mmol) in isopropanol (7.1 mL) and water (1.8 mL) was added silver nitrate (453 mg, 2.67 mmol). The resulting suspension was stirred at 50° C. overnight in the dark. The reaction mixture was filtered through celite, using ethyl acetate as eluent. The filtrate was concentrated to dryness in vacuo to afford a 1:1 mixture of the ester and carboxylic acid of methyl 4-bromo-2-fluoro-6-formylbenzoate (194 mg, 84%) which was used as prepared. LCMS (ES, m/z): 260.9 [M+H]⁺ (ester), 246.9 [M+H]⁺ (acid).

### Synthesis of Intermediate B144

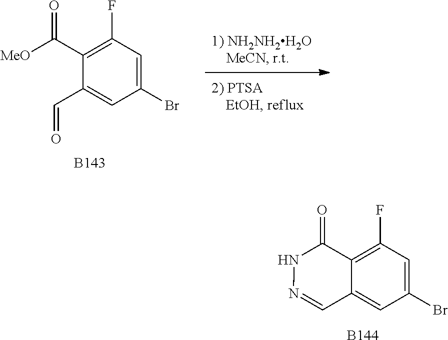

To 6-bromo-8-fluorophthalazin-1(2H)-one (194 mg, 0.743 mmol) was added acetonitrile (5 mL), followed by hydrazine monohydrate (48.4 mg, 0.966 mmol). The reaction mixture was stirred at room temperature for 5 minutes and a precipitate formed. The precipitate was collected by vacuum filtration and the solid was washed with cold acetonitrile (10 mL), then dried under high vacuum for 1 hour. The resulting solid was dissolved in ethanol (5 mL), and to the solution was added p-toluenesulfonic acid (7.1 mg, 0.037 mmol). The reaction mixture was heated under reflux for 72 hours, then diluted with ethyl acetate (50 mL) and washed with saturated NaHCO₃ (30 mL), and brine (2×50 mL). The organic phase was dried over Na₂SO_{4,} filtered, and the filtrate concentrated in vacuo to afford 6-bromo-8-fluorophthalazin-1(2H)-one (210 mg, 97%) as a solid. LCMS (ES, m/z): 242.9 [M+H]⁺.

### Synthesis of Intermediate B145

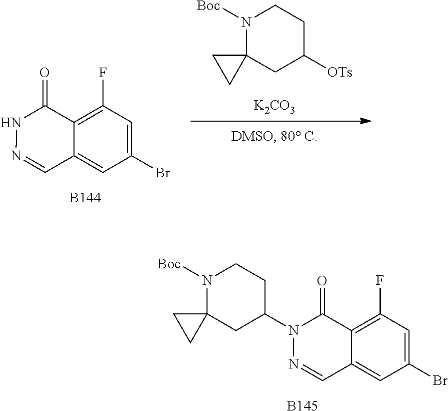

To a mixture of 6-bromo-8-fluorophthalazin-1(2H)-one (210 mg, 0.864 mmol) and tert-butyl 7-(tosyloxy)-4-azaspiro[2.5]octane-4-carboxylate (659 mg, 1.73 mmol) was added DMSO (4.3 mL), followed by K₂CO₃ (512 g, 3.70 mmol). The reaction mixture was heated at 80° C. for 48 hours, then diluted with ethyl acetate (75 mL) and washed with saturated NH₄Cl (50 mL), NaHCO₃ (50 mL), and brine (2×50 mL). The organic phase was dried over Na₂SO₄ and concentrated in vacuo to give a residue. The residue was purified by flash chromatography on silica gel using a gradient of 10-100% ethyl acetate in hexane to afford tert-butyl 7-(6-bromo-8-fluoro-1-oxophthalazin-2(1H)-yl)-4-azaspiro[2.5]octane-4-carboxylate (34 mg, 9%) as a solid. LCMS (ES, m/z): 474.1 [M+Na]⁺.

### Synthesis of Intermediate B146

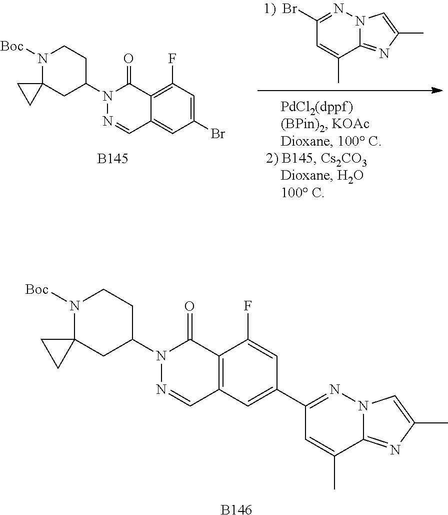 [SMILES: "B145" ; "B146" ; CC(C)(C)OC(=O)N1CCC(n2ncc3cc(Br)cc(F)c3c2=O)CC12CC2 ; Cc1cn2nc(-c3cc(F)c4c(=O)n(C5CCN(C(=O)OC(C)(C)C)C6(CC6)C5)ncc4c3)cc(C)c2n1 ; Cc1cn2nc(Br)cc(C)c2n1]

A mixture of 6-bromo-2,8-dimethylimidazo[1,2-b]pyridazine (43 mg, 0.19 mmol), bis(pinacolato)diboron (50 mg, 0.19 mmol), PdCl₂(dppf) (11 mg, 0.015 mmol), and potassium acetate (45 mg, 0.45 mmol) was dissolved in dioxane (750 μL) and argon was bubbled through the resulting mixture for 10 minutes. The reaction mixture was heated at 100° C. for 1 hour, then cooled. To the reaction mixture was added tert-butyl 7-(6-bromo-8-fluoro-1-oxophthalazin-2(1H)-yl)-4-azaspiro[2.5]octane-4-carboxylate (34 mg, 0.075 mmol) in dioxane (0.6 μL), followed by Cs₂CO₃ (225 mg, 0.690 mmol) in water (200 μL). The reaction mixture was heated at 100° C. for 2 hours, then diluted with ethyl acetate (25 mL) and washed with saturated NaHCO₃ (20 mL) and brine (2×20 mL). The organic phase was then filtered under vacuum, dried over Na₂SO₄, filtered, and the filtrate concentrated in vacuo to give a residue. The residue was purified by flash chromatography on a C18 column using a gradient of 50-100% acetonitrile in water to afford tert-butyl 7-(6-(2, 8-dimethylimidazo[1,2-b]pyridazin-6-yl)-8-fluoro-1-oxophthalazin-2(1H)-yl)-4-azaspiro[2.5]octane-4-carboxylate (13 mg, 33%) as a solid. LCMS (ES, m/z): 519.3 [M+H]⁺

### Synthesis of Compound 312

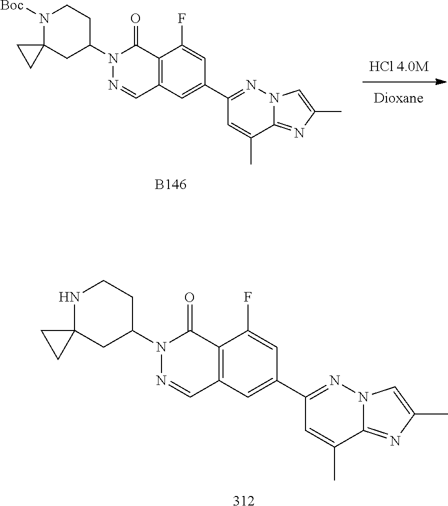 [SMILES: "B146" ; Cc1cn2nc(-c3cc(F)c4c(=O)n(C5CCN(C(=O)OC(C)(C)C)C6(CC6)C5)ncc4c3)cc(C)c2n1 ; Cc1cn2nc(-c3cc(F)c4c(=O)n(C5CCNC6(CC6)C5)ncc4c3)cc(C)c2n1 ; "312"]

To tert-butyl 7-(6-(2,8-dimethylimidazo[1,2-b]pyridazin-6-yl)-8-fluoro-1-oxophthalazin-2(1H)-yl)-4-azaspiro[2.5]octane-4-carboxylate (13 mg, 25 μmol) was added HCl 4.0 M in dioxane (1.5 mL). The reaction mixture was stirred vigorously at room temperature for 2 hours, then concentrated under reduced pressure to give a residue. The residue was partitioned between DCM (20 mL) and 0.25 M NaOH (20 mL) and stirred to neutralize. The phases were separated, and the aqueous phase was extracted with DCM (2×20 mL). The organic phases were combined, washed with brine (30 mL), dried over Na₂SO₄, filtered, and the filtrate concentrated in vacuo to afford 6-(2,8-dimethylimidazo[1,2-b]pyridazin-6-yl)-8-fluoro-2-(4-azaspiro[2.5]octan-7-yl)phthalazin-1(2H)-one (10 mg, 95%) as a a solid. LCMS (ES, m/z): 419.2 [M+H]⁺. ¹H NMR (CDCl₃, 300 MHz): δ 8.26 (1H, d, J=2.5 Hz), 8.06 (2H, s), 7.83 (1H, s), 7.34 (1H, s), 5.37 (1H, m), 3.67-3.79 (1H, m), 3.24 (1H, d,=12.9 Hz), 3.02 (1H, m), 2.78 (3H, s), 2.58 (3H, s), 2.40 (1H, t,=12.2 Hz), 1.98 (2H, m), 0.74 (2H, m), 0.56 (2H, m).

### Example 74

### Synthesis of Compound 313

### Synthesis of Intermediate B147

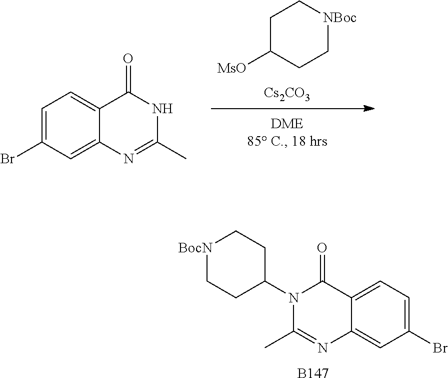

To a solution of 7-bromo-2-methylquinazolin-4(3H)-one (300 mg, 1.19 mmol) in DME (5.8 mL) at 0° C. under nitrogen atmosphere was added Cs₂CO₃ (1.17 g, 3.58 mmol) and tert-butyl 4-((methylsulfonyl)oxy)piperidine-1-carboxylate. The reaction mixture was stirred at 85° C. for 18 h. Ethyl acetate (100 mL) and NH₄Cl (sat) (50 ml) were added. The organic layer was separated, washed with NH₄Cl (sat) (50 ml), NaHCO₃ (sat) (50 ml) and brine (50 mL), dried over MgSO₄, filtered, and the filtrate concentrated under reduced pressure to give a residue. The residue was purified by normal phase chromatography eluting from 20 to 100% ethyl acetate/hexane to afford tert-butyl 4-(7-bromo-2-methyl-4-oxoquinazolin-3(4H)-yl)piperidine-1-carboxylate (91 mg, 18%) as a solid. LCMS (ES, m/z): 422.1, 424.1 [M+H]⁺.

### Synthesis of Intermediate B148

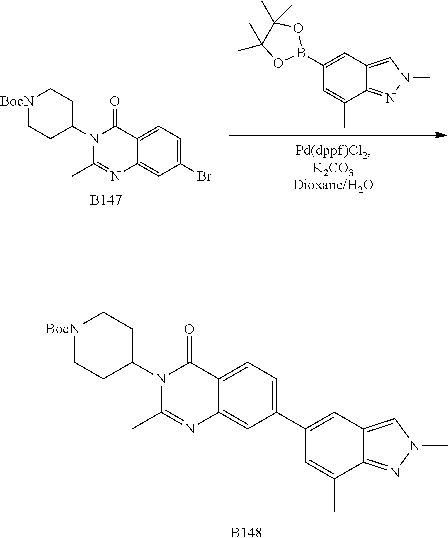

A suspension of the tert-butyl 4-(7-bromo-2-methyl-4-oxoquinazolin-3(4H)-yl)piperidine carboxylate (90 mg, 0.21 mmol) and 2,7-dimethyl-5-(4,4,5,5-tetramethyl-1,3,2-dioxaborolan yl)-2H-indazole (70 mg, 0.26 mmol) in dioxane (2 mL) and water (0.5 mL) was degassed with argon. Then K₂CO₃ (88 mg, 0.64 mmol) was added to the reaction mixture, followed by Pd(dppf)Cl₂-DCM (15 mg, 0.021 mmol). The resulting solution was stirred at 100° C. under an argon atmosphere for 2 h. The reaction mixture was purified using a C18 cartridge eluted with acetonitrile/water (0.1% HCl) from 20 to 80% to give tert-butyl 4-(7-(2,7-dimethyl-2H-indazol-5-yl)-2-methyl-4-oxoquinazolin-3(4H)-yl)piperidine-1-carboxylate (100 mg, 96%) as a solid. LCMS (ES, m/z): 488.3 [M+H]⁺.

### Synthesis of Compound 313

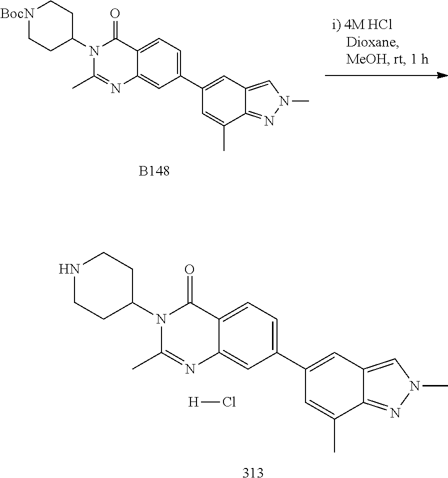

To a solution of tert-butyl 4-(7-(2,7-dimethyl-2H-indazol-5-yl)-2-methyl-4-oxoquinazolin-3(4H)-yl)piperidine-1-carboxylate (100 mg, 0.21 mmol) in methanol (1.0 mL) and DCM (0.5 mL) was added 4M HCl in dioxane (2.0 mL). The reaction mixture was stirred at room temperature for 1 h and a precipitate formed. The precipitate was collected, washed with EtOAc (2.0 mL×2), then dissolved in water (2 mL) and lyophilized to yield 7-(2,7-dimethyl-2H-indazol-5-yl)-2-methyl-3-(piperidin-4-yl)quinazolin-4(3H)-one as a solid (19 mg, 24%). LCMS (ES, m/z): 388.2 [M+H]⁺. ¹H NMR (DMSO-d₆, 400 MHz): δ_{H} 9.23 (1H, br s), 8.57 (1H, br s), 8.45 (1H, s), 8.16 (1H, d, J=8.4 Hz), 8.07 (1H, br s), 7.94-7.96 (2H, m), 7.42 (1H, s), 4.54 (1H, m), 4.20 (3H, s), 3.38 (2H, d, J=11.9 Hz), 3.08 (2H, m), 2.89 (5H, m), 2.58 (3H, s), 2.02 (2H, d, J=12.7 Hz).

### Example 75

### Synthesis of Compound 232

### Synthesis of Intermediate B149

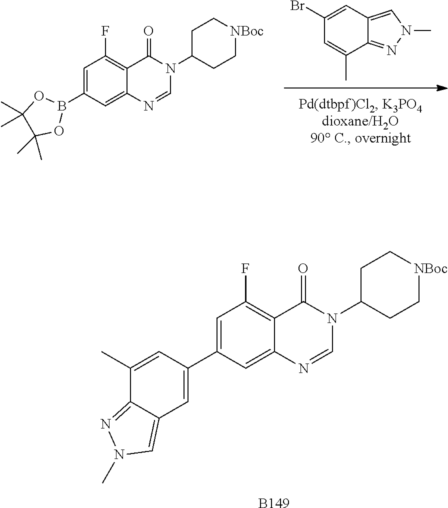

To a stirred mixture of tert-butyl 4-[5-fluoro-4-oxo-7-(4,4,5,5-tetramethyl-1,3,2-dioxaborolan -2-yl)quinazolin-3-yl]piperidine-1-carboxylate (100.00 mg, 0.21 mmol, 1.00 equiv) and 5-bromo-2,7-dimethylindazole (57.06 mg, 0.25 mmol, 1.20 equiv) in dioxane/water (3 mL, 5:1) was added Pd(DtBPF)Cl₂ (13.77 mg, 0.02 mmol, 0.10 equiv) and K₃PO₄ (134.53 mg, 0.63 mmol, 3.00 equiv). The reaction mixture was stirred overnight at 90° C. under nitrogen atmosphere, then extracted with ethyl acetate (3×10 mL). The combined organic layers were washed with saturated NaCl (1×10 mL), dried over anhydrous Na₂SO₄, and filtered. After filtration, the filtrate was concentrated under reduced pressure to give a residue. The residue was purified by silica gel column chromatography, eluted with PE/EA (1:1) to afford tert-butyl 4-[7-(2,7-dimethylindazol-5-yl)-5-fluoro-4-oxoquinazolin-3-yl]piperidine-1-carboxylate (61.00 mg, 58.74%) as a solid. LCMS (ES, m/z):492 [M+H]⁺.

### Synthesis of Compound 232

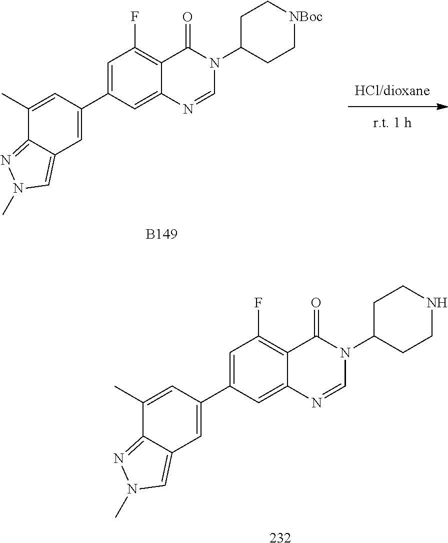 [SMILES: "B149" ; Cc1cc(-c2cc(F)c3c(=O)n(C4CCN(C(=O)OC(C)(C)C)CC4)cnc3c2)cc2cn(C)nc12 ; Cc1cc(-c2cc(F)c3c(=O)n(C4CCNCC4)cnc3c2)cc2cn(C)nc12 ; "232"]

A mixture of tert-butyl 4-[7-(2,7-dimethylindazol-5-yl)-5-fluoro-4-oxoquinazolin-3-yl]piperidine-1-carboxylate (61.00 mg, 0.12 mmol, 1.00 equiv) and HCl (gas) in 1,4-dioxane (5 mL) was stirred for 1 h at room temperature. The resulting mixture was concentrated under reduced pressure to give a residue. The residue was purified by reverse flash chromatography (Column: XBridge Prep OBD C18 Column, 30*150 mm, 5 μm; Mobile Phase A: Water (10 mmol/L NH₄HCO₃), Mobile Phase B: acetonitrile; Flow rate: 60 mL/min; Gradient: 5% B to 35% B in 8 min) to afford 7-(2,7-dimethylindazol-5-yl)-5-fluoro-3-(piperidin-4-yl)quinazolin one (25.90 mg, 53.32%) as a solid. LCMS (ES, m/z):392 [M+H]⁺. ¹H NMR (400 MHz, DMSO-d₆) δ 8.45 (d, J=6.7 Hz, 2H), 8.06 (d, J=1.7 Hz, 1H), 7.79 (d, J=1.7 Hz, 1H), 7.69 (dd, J=12.7, 1.8 Hz, 1H), 7.52 (t, J=1.5 Hz, 1H), 4.67 (tt, J=12.0, 3.9 Hz, 1H), 4.21 (s, 3H), 3.14-3.05 (m, 2H), 2.66-2.55 (m, 2H), 2.59 (s, 3H), 2.27 (s, 1H), 1.89 (qd, J=12.0, 4.0 Hz, 2H), 1.81-1.73 (m, 2H).

### Example 76

### Synthesis of Compound 230

### Synthesis of Intermediate B150

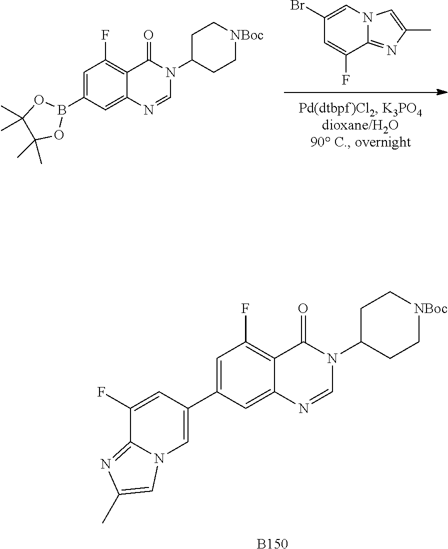 [SMILES: CC(C)(C)OC(=O)N1CCC(n2cnc3cc(B4OC(C)(C)C(C)(C)O4)cc(F)c3c2=O)CC1 ; Cc1cn2cc(Br)cc(F)c2n1 ; "B150" ; Cc1cn2cc(-c3cc(F)c4c(=O)n(C5CCN(C(=O)OC(C)(C)C)CC5)cnc4c3)cc(F)c2n1]

To a stirred mixture of tert-butyl 4-[5-fluoro-4-oxo-7-(4,4,5,5-tetramethyl-1,3,2-dioxaborolan-2-yl) quinazolin-3-yl]piperidine-1-carboxylate (200.00 mg, 0.42 mmol, 1.00 equiv) and 6-bromo-8-fluoro-2-methylimidazo[1,2-a]pyridine (116.13 mg, 0.51 mmol, 1.20 equiv) in dioxane/water (3.00 mL, 5:1) was added Pd(DtBPF)Cl₂ (27.54 mg, 0.04 mmol, 0.10 equiv) and K₃PO₄ (269.06 mg, 1.27 mmol, 3.00 equiv). The reaction mixture was stirred overnight at 90° C. under nitrogen atmosphere. The aqueous layer was extracted with ethyl acetate (3x 10 mL). The combined organic layers were washed with saturated NaCl (1×10 mL), dried over anhydrous Na₂SO₄, and filtered. After filtration, the filtrate was concentrated under reduced pressure to give a residue. The residue was purified by silica gel column chromatography, eluted with PE/EA (1:1) to afford tert-butyl 4-(5-fluoro-7-{8-fluoro-2-methylimidazo[1,2-a]pyridin-6-yl} -4-oxoquinazolin-3-yl)piperidine-1-carboxylate (140.00 mg, 66.87%) as a solid. LCMS (ES, m/z):496 [M+H]⁺.

### Synthesis of Compound 230

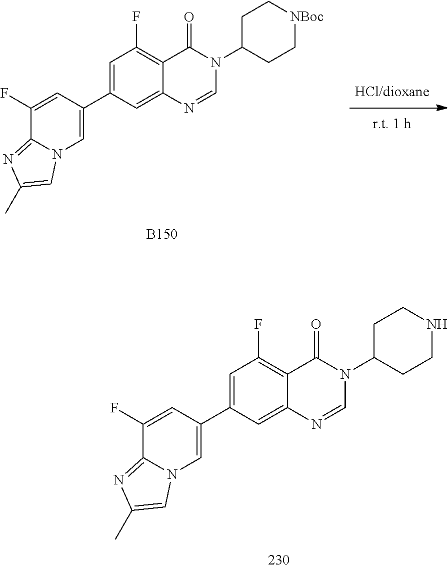 [SMILES: "B150" ; Cc1cn2cc(-c3cc(F)c4c(=O)n(C5CCN(C(=O)OC(C)(C)C)CC5)cnc4c3)cc(F)c2n1 ; Cc1cn2cc(-c3cc(F)c4c(=O)n(C5CCNCC5)cnc4c3)cc(F)c2n1 ; "230"]

A mixture of tert-butyl 4-(5-fluoro-7-{8-fluoro-2-methylimidazo[1,2-a]pyridin-6-yl}-4-oxoquinazolin-3-yl)piperidine-1-carboxylate (70.00 mg, 0.14 mmol, 1.00 equiv) and HCl (gas) in 1,4-dioxane (5 mL) was stirred for 1 h at room temperature. The resulting mixture was concentrated under reduced pressure to give a residue. The residue was purified by reverse flash chromatography (Column: XBridge Prep OBD C18 Column, 30*150 mm, 5 μm; Mobile Phase A: water (10 mmol/L NH₄HCO₃), Mobile Phase B: acetonitrile; Flow rate: 60 mL/min; Gradient: 5% B to 58% B in 8 min) to afford 5-fluoro-7-{8-fluoro-2-methylimidazo [1,2-a]pyridin-6-yl}-3-(piperidin-4-yl)quinazolin-4-one (21.10 mg, 37.77%) as a solid. LCMS (ES, m/z):396 [M+H]⁺. ¹H NMR (400 MHz, DMSO-d₆) δ 9.08 (d, J=1.5 Hz, 1H), 8.49 (s, 1H), 7.90 (d, J=1.8 Hz, 1H), 7.84 (dd, J=3.2, 1.1 Hz, 1H), 7.75 (ddd, J=12.7, 2.8, 1.7 Hz, 1H), 7.73 (ddd, J=12.7, 2.8, 1.7 Hz, 1H), 4.66 (tt, J=12.1, 4.0 Hz, 1H), 3.09 (d, J=12.5 Hz, 2H), 2.60 (td, J=12.2, 2.5 Hz, 2H), 2.39 (d, J=0.9 Hz, 3H), 1.89 (qd, J=11.9, 4.0 Hz, 2H), 1.81-1.74 (m, 2H).

### Example 77

### Synthesis of Compound 314

### Synthesis of Intermediate B151

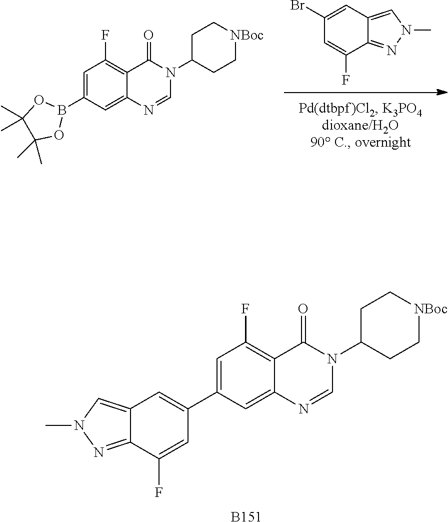 [SMILES: CC(C)(C)OC(=O)N1CCC(n2cnc3cc(B4OC(C)(C)C(C)(C)O4)cc(F)c3c2=O)CC1 ; Cn1cc2cc(Br)cc(F)c2n1 ; "B151" ; Cn1cc2cc(-c3cc(F)c4c(=O)n(C5CCN(C(=O)OC(C)(C)C)CC5)cnc4c3)cc(F)c2n1]

To a stirred mixture of tert-butyl 4-[5-fluoro-4-oxo-7-(4,4,5,5-tetramethyl-1,3,2-dioxaborolan-2-yl) quinazolin-3-yl]piperidine-1-carboxylate (100.00 mg, 0.21 mmol, 1.00 equiv) and 5-bromo-7-fluoro-2-methylindazole (58.07 mg, 0.25 mmol, 1.20 equiv) in dioxane/water (3 mL, 5:1) was added K₃PO₄ (134.53 mg, 0.63 mmol, 3.00 equiv) and Pd(DtBPF)Cl₂ (13.77 mg, 0.02 mmol, 0.10 equiv). The reaction miture was stirred overnight at 90° C. under nitrogen atmosphere. The resulting mixture was extracted with ethyl acetate (3×10 mL). The combined organic layers were washed with saturated NaCl (1×10 mL), dried over anhydrous Na₂SO₄, and filtered. After filtration, the filtrate was concentrated under reduced pressure to give a residue. The residue was purified by silica gel column chromatography, eluted with PE/EA (1:1) to afford tert-butyl 4-(5-fluoro-7-(7-fluoro-2-methyl-2H-indazol-5-yl)-4-oxoquinazolin-3(4H)-yl)piperidine-1-carboxylate (80.00 mg, 76.42%) as a solid. LCMS (ES, m/z):496 [M+H]⁺.

### Synthesis of Compound 314

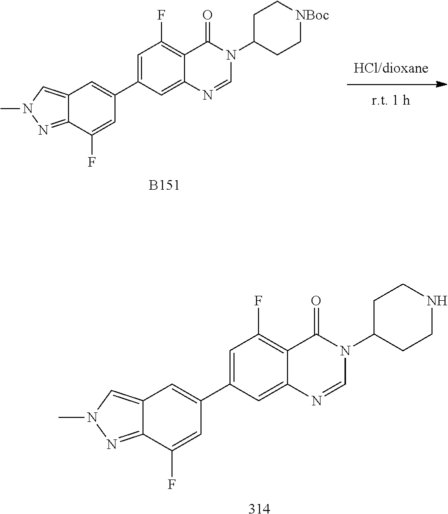 [SMILES: "B151" ; Cn1cc2cc(-c3cc(F)c4c(=O)n(C5CCN(C(=O)OC(C)(C)C)CC5)cnc4c3)cc(F)c2n1 ; Cn1cc2cc(-c3cc(F)c4c(=O)n(C5CCNCC5)cnc4c3)cc(F)c2n1 ; "314"]

A mixture of tert-butyl 4-(5-fluoro-7-(7-fluoro-2-methyl-2H-indazol-5-yl)-4-oxoquinazolin-3(4H)-yl) piperidine-1-carboxylate (80.00 mg, 0.16 mmol, 1.00 equiv) and HCl (gas) in 1,4-dioxane (5 ml) was stirred for 1 h at room temperature. The resulting mixture was concentrated under reduced pressure to give a residue. The residue was purified by reverse flash chromatography (Column: XBridge Prep OBD C18 Column, 30*150 mm, 5 μm; Mobile Phase A: water (10 mmol/L NH₄HCO₃), Mobile Phase B: acetonitrile; Flow rate: 60 mL/min; Gradient: 5% B to 55% B in 8 min) to afford 5-fluoro-7-(7-fluoro-2-methyl-2H-indazol-5-yl)-3-(piperidin-4-yl)quinazolin-4(3H)-one (42.60 mg, 66.73%) as a solid. LCMS (ES, m/z):396 [M+H]⁺. ¹H NMR (400 MHz, DMSO-d₆) δ 8.61 (s, 1H), 8.48 (s, 1H), 8.00 (d, J=1.1 Hz, 1H), 7.88 (d, J=1.8 Hz, 1H), 7.79 (dd, J=12.6, 1.8 Hz, 1H), 7.37 (dd, J=12.1, 1.2 Hz, 1H), 4.67 (tt, J=12.1, 4.0 Hz, 1H), 4.23 (s, 3H), 3.13-3.06 (m, 2H), 2.61 (td, J=12.2, 2.5 Hz, 2H), 1.89 (qd, J=11.9, 4.0 Hz, 2H), 1.77 (dd, J=12.8, 3.7 Hz, 2H).

### Example 78

### Synthesis of Compound 315

### Synthesis of Intermediate B152

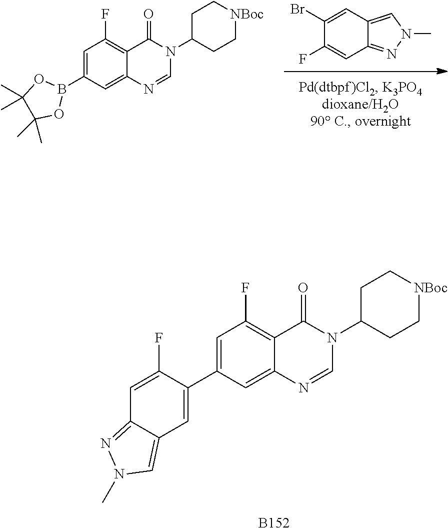 [SMILES: CC(C)(C)OC(=O)N1CCC(n2cnc3cc(B4OC(C)(C)C(C)(C)O4)cc(F)c3c2=O)CC1 ; Cn1cc2cc(Br)c(F)cc2n1 ; "B152" ; Cn1cc2cc(-c3cc(F)c4c(=O)n(C5CCN(C(=O)OC(C)(C)C)CC5)cnc4c3)c(F)cc2n1]

To a stirred mixture of tert-butyl 4-[5-fluoro-4-oxo-7-(4,4,5,5-tetramethyl-1,3,2-dioxaborolan-2-yl) quinazolin-3-yl]piperidine-1-carboxylate (100.00 mg, 0.21 mmol, 1.00 equiv) and 5-bromo-6-fluoro-2-methylindazole (58.07 mg, 0.25 mmol, 1.20 equiv) in dioxane/water (3 mL, 5:1) was added Pd(DtBPF)Cl₂ (27.54 mg, 0.04 mmol, 0.10 equiv) and K₃PO₄ (134.53 mg, 0.63 mmol, 3.00 equiv). The reaction mixture was stirred overnight at 90° C. under nitrogen atmosphere. The resulting mixture was extracted with ethyl acetate (3×10 mL). The combined organic layers were washed with saturated NaCl (1×10 mL), dried over anhydrous Na₂SO₄, and filtered. After filtration, the filtrate was concentrated under reduced pressure to give a residue. The residue was purified by silica gel column chromatography, eluted with PE/EA (1:1) to afford tert-butyl 4-[5-fluoro-7-(6-fluoro-2-methylindazol-5-yl)-4-oxoquinazolin-3-yl]piperidine-1-carboxylate (60.00 mg, 57.31%) as a solid. LCMS (ES, m/z):496 [M+H]⁺.

### Synthesis of Compound 315

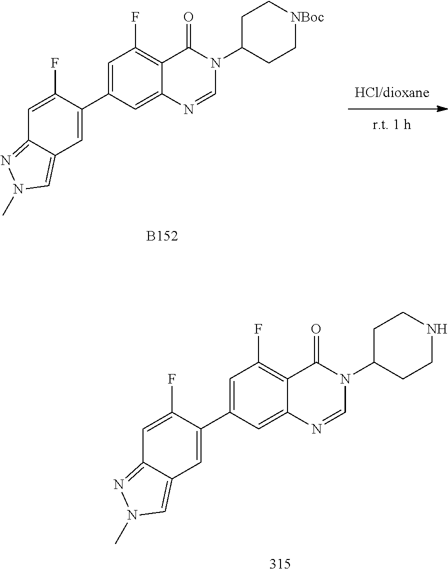 [SMILES: "B152" ; Cn1cc2cc(-c3cc(F)c4c(=O)n(C5CCN(C(=O)OC(C)(C)C)CC5)cnc4c3)c(F)cc2n1 ; Cn1cc2cc(-c3cc(F)c4c(=O)n(C5CCNCC5)cnc4c3)c(F)cc2n1 ; "315"]

A mixture of tert-butyl 4-[5-fluoro-7-(6-fluoro-2-methylindazol-5-yl)-4-oxoquinazolin-3-yl]piperidine-1-carboxylate (60.00 mg, 0.12 mmol, 1.00 equiv) and HCl (gas) in 1,4-dioxane (5 mL) was stirred for 1 h at room temperature. The resulting mixture was concentrated under reduced pressure to give a residue. The residue was purified by reverse flash chromatography (Column: Xselect CSH OBD Column 30*150 mm Sum, n; Mobile Phase A: water (10 mmol/L NH₄HCO₃), Mobile Phase B: acetonitrile; Flow rate: 60 mL/min; Gradient: 5% B to 36% B in 8 min) to afford 5-fluoro-7-(6-fluoro-2-methylindazol-5-yl)-3-(piperidin-4-yl)quinazolin-4-one (34.90 mg, 72.89%) as a solid. LCMS (ES, m/z):396 [M+H]⁺. ¹H NMR (400 MHz, DMSO-d₆) δ 8.50 (d, J=16.7 Hz, 2H), 8.08 (d, J=7.9 Hz, 1H), 7.66 (d, J=1.9 Hz, 1H), 7.57(m, 1H), 7.48 (m, 1H), 4.68 (tt, J=12.0, 3.9 Hz, 1H), 4.20 (s, 3H), 3.13 — 3.06 (m, 2H), 2.61 (td, J=12.2, 2.5 Hz, 2H), 1.89 (qd, J=11.9, 4.0 Hz, 2H), 1.77 (d, J=10.9 Hz, 2H).

### Example 79

### Synthesis of Compound 316

### Synthesis of Intermediate B153

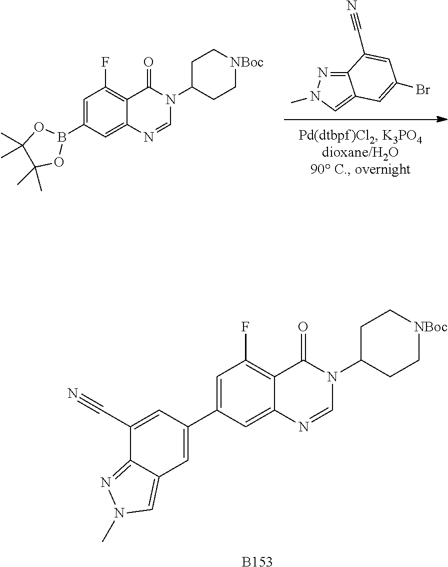 [SMILES: CC(C)(C)OC(=O)N1CCC(n2cnc3cc(B4OC(C)(C)C(C)(C)O4)cc(F)c3c2=O)CC1 ; Cn1cc2cc(Br)cc(C#N)c2n1 ; "B153" ; Cn1cc2cc(-c3cc(F)c4c(=O)n(C5CCN(C(=O)OC(C)(C)C)CC5)cnc4c3)cc(C#N)c2n1]

To a mixture of tert-butyl 4-[5-fluoro-4-oxo-7-(4,4,5,5-tetramethyl-1,3,2-dioxaborolan-2-yl) quinazolin-3-yl]piperidine-1-carboxylate (100.00 mg, 0.21 mmol, 1.00 equiv) and 5-bromo methylindazole-7-carbonitrile (59.85 mg, 0.25 mmol, 1.20 equiv) in dioxane/water (3 mL, 5:1) was added Pd(DtBPF)Cl₂ (13.77 mg, 0.02 mmol, 0.10 equiv) and K₃PO₄ (134.53 mg, 0.63 mmol, 3.00 equiv) in portions at 90° C. under nitrogen atmosphere. The resulting mixture was extracted with ethyl acetate (3×10 mL). The combined organic layers were washed with saturated NaCl (1×10 mL), dried over anhydrous Na₂SO₄, and filtered. After filtration, the filtrate was concentrated under reduced pressure to give a residue. The residue was purified by silica gel column chromatography, eluted with PE/EA (1:1) to afford tert-butyl 4-[7-(7-cyano-2-methylindazol-5-yl)-5-fluoro-4-oxoquinazolin-3-yl]piperidine-1-carboxylate (65.00 mg, 61.22%) as a solid. LCMS (ES, m/z):503 [M+H]⁺.

### Synthesis of Compound 316

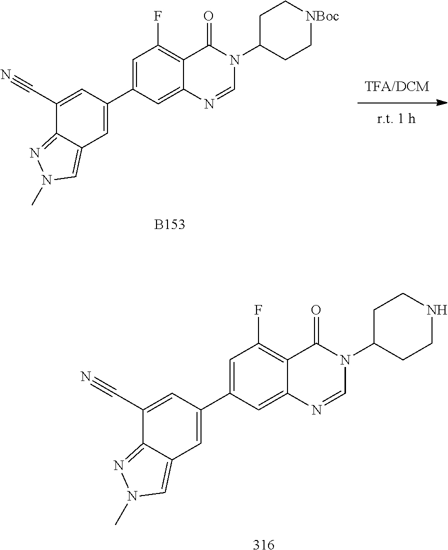 [SMILES: "B153" ; Cn1cc2cc(-c3cc(F)c4c(=O)n(C5CCN(C(=O)OC(C)(C)C)CC5)cnc4c3)cc(C#N)c2n1 ; Cn1cc2cc(-c3cc(F)c4c(=O)n(C5CCNCC5)cnc4c3)cc(C#N)c2n1 ; "316"]

A mixture of tert-butyl 4-[7-(7-cyano-2-methylindazol-5-yl)-5-fluoro-4-oxoquinazolin-3-yl]piperidine-1-carboxylate (65.00 mg, 0.13 mmol, 1.00 equiv) and TFA (0.5 mL) in DCM (3 mL) was stirred for 1 h at room temperature. The resulting mixture was concentrated under reduced pressure to give a residue. The residue was purified by reverse flash chromatography (Column:) (Bridge Prep OBD C18 Column, 30*150 mm, 5 μm; Mobile Phase A: water (10 mmol/L NH₄HCO₃), Mobile Phase B: acetonitrile; Flow rate: 60 mL/min; Gradient: 5% B to 45% B in 8 min) to afford 5-[5-fluoro-4-oxo-3-(piperidin-4-yl)quinazolin-7-yl]-2-methylindazole-7-carbonitrile (20.10 mg, 38.62%) as a solid. LCMS (ES, m/z):403 [M+H]⁺. ¹H NMR (400 MHz, DMSO-d₆) δ 8.75 (s, 1H), 8.68 (d, J=1.8 Hz, 1H), 8.51-8.43 (m, 2H), 7.92 (d, J=1.7 Hz, 1H), 7.82 (dd, J=12.6, 1.8 Hz, 1H), 4.67 (tt, J=12.1, 3.9 Hz, 1H), 4.29 (s, 3H), 3.13-3.06 (m, 2H), 2.61 (td, J=12.1, 2.4 Hz, 2H), 1.89 (qd, J=12.0, 4.0 Hz, 2H), 1.82-1.73 (m, 2H).

### Example 80

### Synthesis of Compound 317

### Synthesis of Intermediate B154

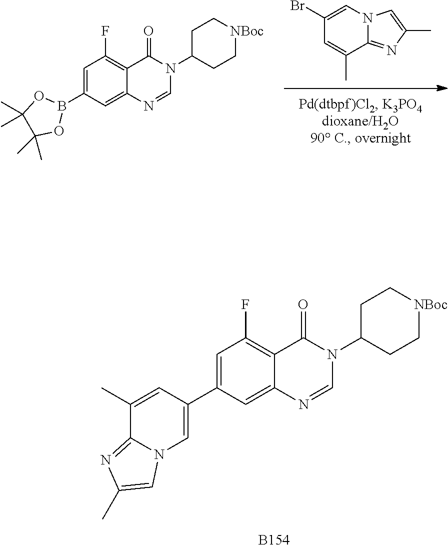 [SMILES: CC(C)(C)OC(=O)N1CCC(n2cnc3cc(B4OC(C)(C)C(C)(C)O4)cc(F)c3c2=O)CC1 ; Cc1cn2cc(Br)cc(C)c2n1 ; "B154" ; Cc1cn2cc(-c3cc(F)c4c(=O)n(C5CCN(C(=O)OC(C)(C)C)CC5)cnc4c3)cc(C)c2n1]

To a stirred mixture of tert-butyl 4-[5-fluoro-4-oxo-7-(4,4,5,5-tetramethyl-1,3,2-dioxaborolan-2-yl) quinazolin-3-yl]piperidine-1-carboxylate (100.00 mg, 0.21 mmol, 1.00 equiv) and 6-bromo-2,8-dimethylimidazo[1,2-a]pyridine (57.06 mg, 0.25 mmol, 1.20 equiv) in dioxane/water (3 mL, 5:1) was added Pd(DtBPF)Cl₂ (13.77 mg, 0.02 mmol, 0.10 equiv) and K₃PO₄ (134.53 mg, 0.63 mmol, 3.00 equiv) in portions at 90° C. under nitrogen atmosphere. The resulting mixture was extracted with ethyl acetate (3 ×10 mL). The combined organic layers were washed with saturated NaCl (1×10 mL), dried over anhydrous Na₂SO₄, and filtered. After filtration, the filtrate was concentrated under reduced pressure to give a residue. The residue was purified by silica gel column chromatography, eluted with PE/EA (1:1) to afford tert-butyl 4-(7-{2,8-dimethylimidazo[1,2-a]pyridin-6-yl}-5-fluoro-4-oxoquinazolin-3-yl)piperidine-1-carboxylate (50.00 mg, 48.15%) as a solid. LCMS (ES, m/z):492 [M+H]⁺.

### Synthesis of Compound 317

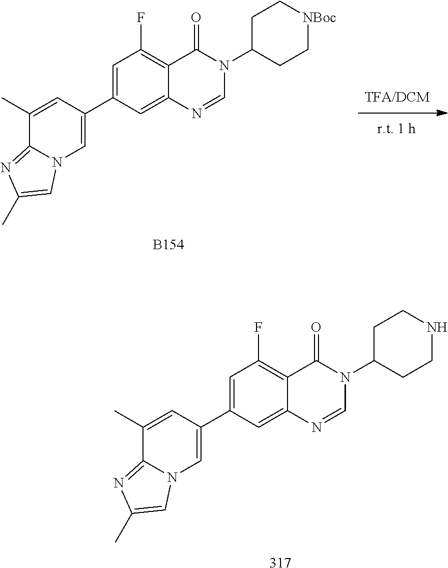 [SMILES: "B154" ; Cc1cn2cc(-c3cc(F)c4c(=O)n(C5CCN(C(=O)OC(C)(C)C)CC5)cnc4c3)cc(C)c2n1 ; Cc1cn2cc(-c3cc(F)c4c(=O)n(C5CCNCC5)cnc4c3)cc(C)c2n1 ; "317"]

A mixture of tert-butyl 4-(7-{2,8-dimethylimidazo[1,2-a]pyridin-6-yl}-5-fluoro-4-oxoquinazolin-3-yl)piperidine-1-carboxylate (50.00 mg, 0.10 mmol, 1.00 equiv) and TFA (0.5 mL) in DCM (3 mL) was stirred for 1 h at room temperature. The resulting mixture was concentrated under reduced pressure to give a residue. The residue was purified by reverse flash chromatography (Column: XBridge Prep OBD C18 Column, 30*150 mm, 5 μm; Mobile Phase A: water (10 mmol/L NH₄HCO₃), Mobile Phase B: acetonitrile; Flow rate: 60 mL/min; Gradient: 5% B to 35% B in 8 min) to afford 7-{2,8-dimethylimidazo[1,2-a]pyridin-6-yl}-5-fluoro-3-(piperidin-4-yl)quinazolin-4-one (17.80 mg, 44.71%) as a solid. LCMS (ES, m/z):392 [M+H]⁺. ¹H NMR (400 MHz, DMSO-d₆) δ 9.00 (d, J=2.0 Hz, 1H), 8.48 (s, 1H), 7.85 (d, J=1.7 Hz, 1H), 7.71 (dd, J=14.6, 1.5 Hz, 2H), 7.57 (t, J=1.6 Hz, 1H), 4.67 (tt, J=12.0, 3.9 Hz, 1H), 3.09 (d, J=12.5 Hz, 2H), 2.66-2.56 (m, 2H), 2.54 (s, 3H), 2.37 (s, 3H), 1.89 (qd, J=12.0, 4.0 Hz, 2H), 1.78 (t, J=6.8 Hz, 2H).

### Example 81

### Synthesis of Compound 318

### Synthesis of Intermediate B155

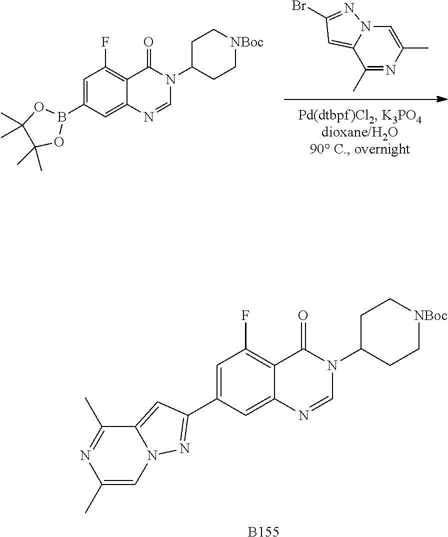 [SMILES: CC(C)(C)OC(=O)N1CCC(n2cnc3cc(B4OC(C)(C)C(C)(C)O4)cc(F)c3c2=O)CC1 ; Cc1cn2nc(Br)cc2c(C)n1 ; "B155" ; Cc1cn2nc(-c3cc(F)c4c(=O)n(C5CCN(C(=O)OC(C)(C)C)CC5)cnc4c3)cc2c(C)n1]

To a stirred mixture of tert-butyl 4-[5-fluoro-4-oxo-7-(4,4,5,5-tetramethyl-1,3,2-dioxaborolan-2-yl) quinazolin-3-yl]piperidine-1-carboxylate (100.00 mg, 0.21 mmol, 1.00 equiv) and 2-bromo-4,6-dimethylpyrazolo[1,5-a]pyrazine (57.31 mg, 0.25 mmol, 1.20 equiv) in dioxane/water (3 mL, 5:1) was added Pd(DtBPF)Cl₂ (13.77 mg, 0.02 mmol, 0.10 equiv) and K₃PO₄ (134.53 mg, 0.63 mmol, 3.00 equiv). The reaction mixture was stirred overnight at 90° C. under nitrogen atmosphere. The resulting mixture was extracted with ethyl acetate (3×10 mL). The combined organic layers were washed with saturated NaCl (1×10 mL), dried over anhydrous Na₂SO₄, and filtered. After filtration, the filtrate was concentrated under reduced pressure to give a residue. The residue was purified by silica gel column chromatography, eluted with PE/EA (1:1) to afford tert-butyl 4-(7-{4,6-dimethylpyrazolo[1,5-a]pyrazin-2-yl}-5-fluoro-4-oxoquinazolin-3-yl)piperidine-1-carboxylate (75.00 mg, 72.08%) as a solid. LCMS (ES, m/z):493 [M+H]⁺.

### Synthesis of Compound 318

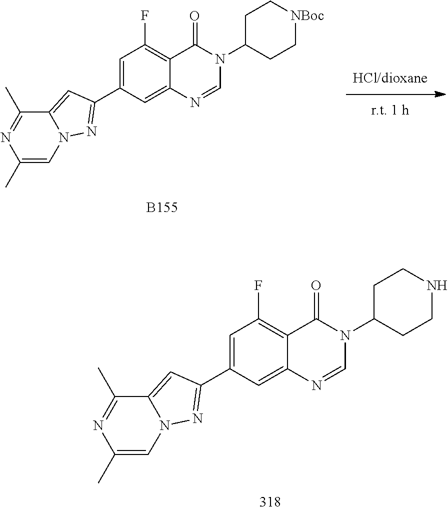 [SMILES: "B155" ; Cc1cn2nc(-c3cc(F)c4c(=O)n(C5CCN(C(=O)OC(C)(C)C)CC5)cnc4c3)cc2c(C)n1 ; Cc1cn2nc(-c3cc(F)c4c(=O)n(C5CCNCC5)cnc4c3)cc2c(C)n1 ; "318"]

A mixture of tert-butyl 4-(7-{4, 6-dimethylpyrazolo[1,5-a]pyrazin-2-yl}-5-fluoro-4-oxoquinazolin-3-yl) piperidine-1-carboxylate (75.00 mg, 0.15 mmol, 1.00 equiv) and HCl (gas) in 1,4-dioxane (5 mL) was stirred for 1 h at room temperature. The resulting mixture was concentrated under reduced pressure to give a residue. The residue was purified by reverse flash chromatography (Column: XBridge Prep OBD C18 Column, 30*150 mm, 5 μm; Mobile Phase A: water (10 mmol/L NH₄HCO₃), Mobile Phase B: acetonitrile; Flow rate: 60 mL/min; Gradient: 5% B to 35% B in 8 min) to afford 7-{4,6-dimethylpyrazolo[1,5-a]pyrazin-2-yl}-5-fluoro-3-(piperidin-4-yl)quinazolin-4-one (16.90 mg, 28.28%) as a solid. LCMS (ES, m/z):393 [M+H]⁺. ¹H NMR (400 MHz, DMSO-d₆) δ 8.55 (d, J=1.4 Hz, 1H), 8.49 (s, 1H), 8.10 (d, J=1.6 Hz, 1H), 7.88 (dd, J=12.1, 1.6 Hz, 1H), 7.77 (d, J=1.0 Hz, 1H), 4.67 (ddd, J=12.1, 8.2, 4.0 Hz, 1H), 3.09 (d, J=12.1 Hz, 2H), 2.72 (s, 3H), 2.66-2.55 (m, 2H), 2.44 (d, J=1.0 Hz, 3H), 2.33 (m, 1H),1.89 (qd, J=11.9, 4.0 Hz, 2H), 1.78 (d, J=10.9 Hz, 2H).

### Example 82

### Synthesis of Compound 319

### Synthesis of Intermediate B156

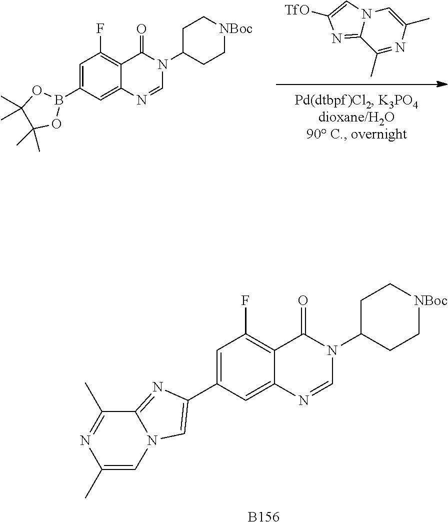 [SMILES: CC(C)(C)OC(=O)N1CCC(n2cnc3cc(B4OC(C)(C)C(C)(C)O4)cc(F)c3c2=O)CC1 ; Cc1cn2cc(OS(=O)(=O)C(F)(F)F)nc2c(C)n1 ; "B156" ; Cc1cn2cc(-c3cc(F)c4c(=O)n(C5CCN(C(=O)OC(C)(C)C)CC5)cnc4c3)nc2c(C)n1]

To a stirred mixture of tert-butyl 4-[5-fluoro-4-oxo-7-(4,4,5,5-tetramethyl-1,3,2-dioxaborolan-2-yl) quinazolin-3-yl]piperidine-1-carboxylate (100.00 mg, 0.21 mmol, 1.00 equiv) and 8-methylimidazo[1,2-a]pyrazin-2-yl trifluoromethanesulfonate (71.29 mg, 0.25 mmol, 1.20 equiv) in dioxane/water (3 mL, 5:1) was added Pd(DtBPF)Cl₂ (13.77 mg, 0.02 mmol, 0.10 equiv) and K₃PO₄ (134.53 mg, 0.63 mmol, 3.00 equiv). The reaction mixture was stirred overnight at 90° C. under nitrogen atmosphere. The resulting mixture was extracted with ethyl acetate (3×10 mL). The combined organic layers were washed with saturated NaCl (1×10 mL), dried over anhydrous Na₂SO₄, and filtered. After filtration, the filtrate was concentrated under reduced pressure to give a residue. The residue was purified by silica gel column chromatography, eluted with PE/EA (1:1) to afford tert-butyl 4-(5-fluoro-7-{8-methylimidazo[1,2-a]pyrazin-2-yl}-4-oxoquinazolin-3-yl)piperidine-1-carboxylate (80.00 mg, 79.13%) as a solid. LCMS (ES, m/z):493 [M+H]⁺.

### Synthesis of Compound 319

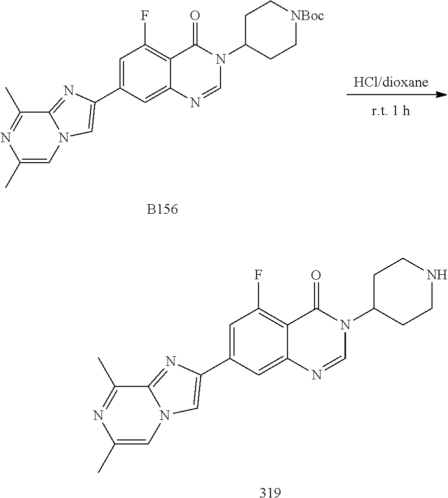 [SMILES: "B156" ; Cc1cn2cc(-c3cc(F)c4c(=O)n(C5CCN(C(=O)OC(C)(C)C)CC5)cnc4c3)nc2c(C)n1 ; Cc1cn2cc(-c3cc(F)c4c(=O)n(C5CCNCC5)cnc4c3)nc2c(C)n1 ; "319" ; "HCl/dioxane"]

A mixture of tert-butyl 4-(5-fluoro-7-{8-methylimidazo[1,2-a]pyrazin-2-yl}-4-oxoquinazolin-3-yl) piperidine-1-carboxylate (80.00 mg, 0.17 mmol, 1.00 equiv) and HCl (gas) in 1,4-dioxane (5 mL) was stirred for 1 h at room temperature. The resulting mixture was concentrated under reduced pressure to give a residue. The residue was purified by reverse flash chromatography (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 5% B to 32% B in 8 min) to afford 5-fluoro-7-{8-methylimidazo[1,2-a]pyrazin-2-yl}-3-(piperidin-4-yl)quinazolin-4-one (17.80 mg, 28.14%) as a solid. LCMS (ES, m/z):393 [M+H]⁺. ¹H NMR (400 MHz, DMSO-d₆) δ 8.75 (s, 1H), 8.48 (s, 1H), 8.28 (s, 1H), 8.10 (d, J=1.5 Hz, 1H), 7.90 (dd, J=12.2, 1.6 Hz, 1H), 4.66 (tt, J=12.1, 4.0 Hz, 1H), 3.13-3.05 (m, 2H), 2.77 (s, 3H), 2.74 (s, 2H), 2.40 (d, J=1.0 Hz, 3H), 1.89 (qd, J=11.9, 4.0 Hz, 2H), 1.82-1.74 (m, 2H).

### Example 83

### Synthesis of Compound 320

### Synthesis of Intermediate B157

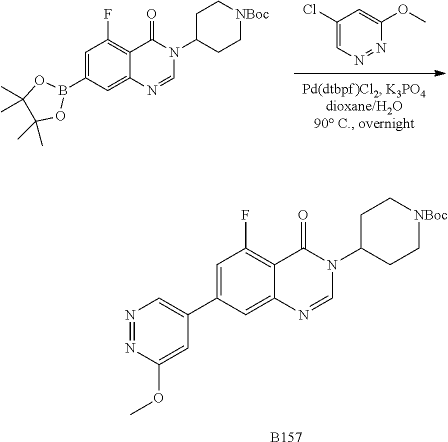

To a stirred mixture of tert-butyl 4-[5-fluoro-4-oxo-7-(4,4,5,5-tetramethyl-1,3,2-dioxaborolan -2-yl)quinazolin-3-yl]piperidine-1-carboxylate (100.00 mg, 0.21 mmol, 1.00 equiv) and 5-chloro-3-methoxypyridazine (36.65 mg, 0.25 mmol, 1.20 equiv) in dioxane/water (3 mL, 5:1) was added K₃PO₄ (134.53 mg, 0.633 mmol, 3 equiv) and Pd(DtBPF)Cl₂ (13.77 mg, 0.02 mmol, 0.10 equiv) in portions at 90° C. under nitrogen atmosphere. The resulting mixture was extracted with ethyl acetate (3×10 mL). The combined organic layers were washed with sat. NaCl (1×10 mL), dried over anhydrous Na₂SO₄, and filtered. After filtration, the filtrate was concentrated under reduced pressure to give a residue. The residue was purified by silica gel column chromatography, eluted with PE/EA (1:1) to afford tert-butyl 4-[5-fluoro-7-(6-methoxypyridazin-4-yl) -4-oxoquinazolin-3-yl]piperidine-1-carboxylate (85.00 mg, 88.33%) as a solid. LCMS (ES, m/z):456 [M+H]⁺.

### Synthesis of Compound 320

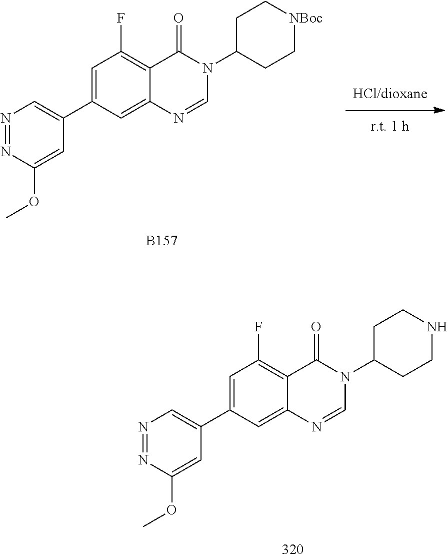 [SMILES: "B157" ; COc1cc(-c2cc(F)c3c(=O)n(C4CCN(C(=O)OC(C)(C)C)CC4)cnc3c2)cnn1 ; COc1cc(-c2cc(F)c3c(=O)n(C4CCNCC4)cnc3c2)cnn1 ; "320"]

A mixture of tert-butyl 4-[5-fluoro-7-(6-methoxypyridazin-4-yl)-4-oxoquinazolin-3-yl]piperidine-1-carboxylate (85.00 mg, 0.19 mmol, 1.00 equiv) and HCl (gas) in 1,4-dioxane (5 mL) was stirred for 1 h at room temperature. The resulting mixture was concentrated under reduced pressure to give a residue. The residue was purified by reverse flash chromatography (Column:) (Bridge Prep OBD C18 Column, 30*150 mm, 5 μm; Mobile Phase A: water (10 mmol/L NH₄HCO₃), Mobile Phase B: acetonitrile; Flow rate: 60 mL/min; Gradient: 5% B to 35% B in 8 min) to afford 5-fluoro-7-(6-methoxypyridazin-4-yl)-3-(piperidin-4-yl)quinazolin-4-one (23.40 mg, 35.29%) as a solid. LCMS (ES, m/z):356 [M+H]⁺. ¹H NMR (400 MHz, DMSO-d₆) δ 9.47 (d, J=1.9 Hz, 1H), 8.52 (s, 1H), 8.09 (d, J=1.7 Hz, 1H), 8.00-7.89 (m, 1H), 7.75 (d, J=2.0 Hz, 1H), 4.66 (tt, J=12.0, 3.9 Hz, 1H), 4.11 (s, 3H), 3.13-3.05 (m, 2H), 2.60 (td, J=12.1, 2.4 Hz, 2H), 1.89 (qd, J=11.9, 4.0 Hz, 2H), 1.82-1.73 (m, 2H).

### Example 84

### Synthesis of Compound 321

### Synthesis of Intermediate B158

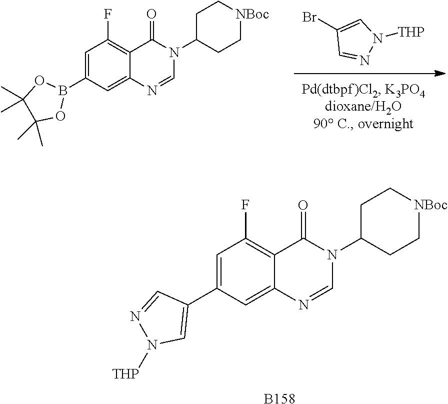

To a stirred mixture of tert-butyl 4-[5-fluoro-4-oxo-7-(4,4,5,5-tetramethyl -1,3,2-dioxaborolan-2-yl) quinazolin-3-yl]piperidine-1-carboxylate (100.00 mg, 0.21 mmol, 1.00 equiv) and 4-bromo-1-(oxan-2-yl) pyrazole (58.58 mg, 0.25 mmol, 1.20 equiv) in dioxane/water (3 mL, 5:1) was added K₃PO₄ (134.53 mg, 0.63 mmol, 3.00 equiv) and Pd(DtBPF)Cl₂ (13.77 mg, 0.02 mmol, 0.10 equiv). The reaction mixture was stirred overnight at 90° C. under nitrogen atmosphere. The resulting mixture was extracted with ethyl acetate (3×10 mL). The combined organic layers were washed with saturated NaCl (1×10 mL), dried over anhydrous Na₂SO₄, and filtered. After filtration, the filtrate was concentrated under reduced pressure to give a residue. The residue was purified by silica gel column chromatography, eluted with PE/EA (1:1) to afford tert-butyl 4-{5-fluoro-7-[1-(oxan-2-yl) pyrazol-4-yl]-4-oxoquinazolin-3-yl}piperidine-1-carboxylate (53.00 mg, 50.42%) as a solid. LCMS (ES, m/z):498 [M+H]⁺.

### Synthesis of Compound 321

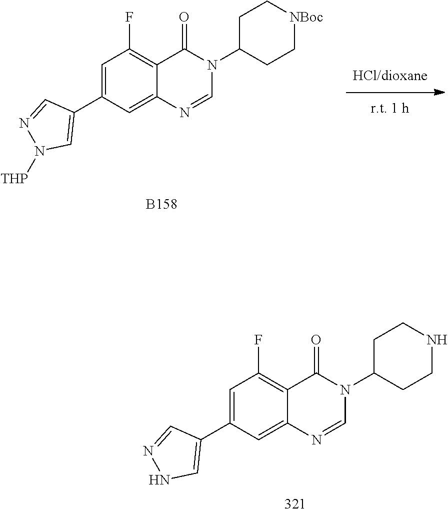

A mixture of tert-butyl 4-{5-fluoro-7-[1-(oxan-2-yl)pyrazol-4-yl]-4-oxoquinazolin-3-yl}piperidine -1-carboxylate (53.00 mg, 0.17 mmol, 1.00 equiv) and HCl (gas) in 1,4-dioxane (5 mL) was stirred for 1 h at room temperature. The resulting mixture was concentrated under reduced pressure to give a residue. The residue was purified by reverse flash chromatography (Column: YMC-Actus Triart C18, 30*150 mm, 5 μm; Mobile Phase A: water (10 mmol/L NH₄HCO₃), Mobile Phase B: acetonitrile; Flow rate: 60 mL/min; Gradient: 5% B to 40% B in 8 min) to afford 5-fluoro-3-(piperidin-4-yl)-7-(1H-pyrazol-4-yl)quinazolin-4-one (13.80 mg, 41.35%) as a solid. LCMS (ES, m/z):314 [M+H]+. ¹H NMR (400 MHz, DMSO-d₆) δ 13.00 (s, 1H), 8.31 (s, 1H), 8.23 (s, 2H), 7.68 (d, J=1.6 Hz, 1H), 7.53 (dd, J=12.6, 1.7 Hz, 1H), 4.64 (tt, J=12.1, 4.0 Hz, 1H), 3.10 (d, J=12.4 Hz, 2H), 2.67 — 2.57 (m, 2H), 1.95(m, 1H), 1.73 (m, 2H), 1.71 (m, 2H).

### Example 85

### Synthesis of Compound 238

### Synthesis of Intermediate B159

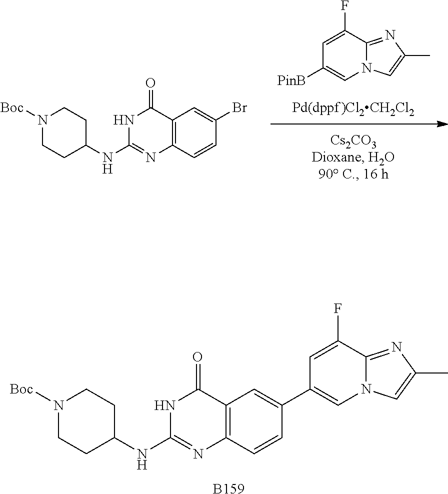

Argon was bubbled into a mixture of tert-butyl 4-((6-bromo-4-oxo-3,4-dihydroquinazolin-2-yl)amino)piperidine-1-carboxylate (90 mg, 0.21 mmol), 8-fluoro-2-methyl-6-(4,4,5,5-tetramethyl-1,3,2-dioxaborolan-2-yl)imidazo[1,2-a]pyridine (57.9 mg, 0.21 mmol), and dioxane (2.1 mL). To the reaction mixture was added water (0.1 mL), followed by Cs₂CO₃ (174 mg, 0.53 mjmmol) and Pd(dppf)Cl₂.CH₂Cl₂ (17.4 mg, 0.021 mmol). The reaction mixture was purged with argon for 10 min, then heated at 95° C. for 16 h. DMF was added to the cooled reaction mixture followed by dropwise addition of 1 N HCl to pH 7. The reaction mixture was filtered through Celite, rinsed with DMF, and the filtrate was concentrated in vacuo to give a residue. The residue was purified by silica gel chromatography using a gradient of 80 to 100% of ethyl acetate in hexane to provide tert-butyl 4-((6-(8-fluoro-2-methylimidazo[1,2-a]pyridin-6-yl)-4-oxo-3,4-dihydroquinazolin-2-yl)amino)piperidine-1-carboxylate (62 mg, 59%). LCMS (ES, m/z): 493.0 [M+H]⁺. ¹H NMR (DMSO-d₆, 400 MHz): δ_{H} 10.75 (1H, s), 8.81 (1H, s), 8.16 (1H, d, J=2.3 Hz), 7.91 (1H, dd, J=8.5, 2.3 Hz), 7.81 (1H, d, J=2.9 Hz), 7.51 (1H, d, J=12.6 Hz), 7.35 (1H, d, J=8.6 Hz), 6.39 (1H, s), 4.00 (1H, br s), 3.84 (2H, d, J=13.3 Hz), 2.96 (2H, br s), 2.36 (3H, s), 1.93 (2H, d, J=12.3 Hz), 1.40 (9H, s), 1.31-1.36 (2H, m).

### Synthesis of Compound 238

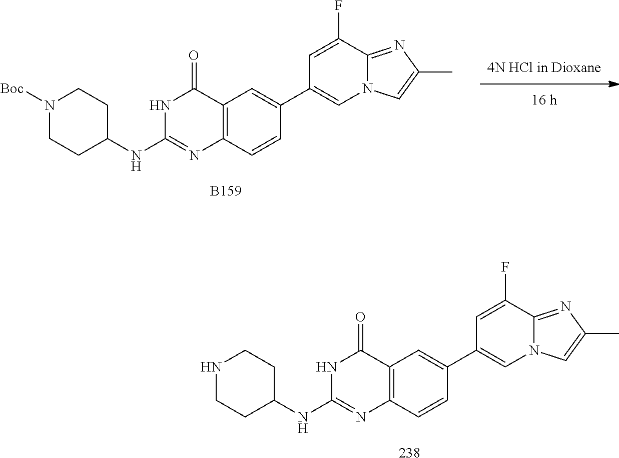

To tert-butyl 4-((6-(8-fluoro-2-methylimidazo[1,2-a]pyridin-6-yl)-4-oxo-3,4-dihydroquinazolin-2-yl)amino)piperidine-1-carboxylate (62 mg, 0.13 mmol) was added 4 N HCl in dioxane (2 mL). The suspension was stirred for 12 h. The reaction was concentrated, dissolved in water, and filtered through a 40 μm syringe filter. The solution was neutralized to pH 6-7 with 1 N NaOH. A precipitate formed and was collected by filtration, rinsed with water, and dried. The solid was purified by silica gel chromatography using a gradient from 0 to 30% of methanol in 2% Et₃N in CH₂Cl₂ to afford 6-(8-fluoro-2-methylimidazo[1,2-a]pyridin-6-yl)-2-(piperidin ylamino)quinazolin-4(3H)-one (22 mg, 45%). LCMS (ES, m/z): 393.1 [M+H]⁺.¹H NMR (DMSO-d₆, 400 MHz): δ_{H} 8.81 (1H, s), 8.17 (1H, s), 7.92 (1H, d, J=8.6 Hz), 7.80 (1H, s), 7.51 (1H, d, J=12.6 Hz), 7.33 (1H, d, J=8.6 Hz), 6.84 (1H, s), 4.07 (1H, br s), 3.23-3.25 (2H, m), 3.00 (2H, t, J=11.7 Hz), 2.35 (3H, s), 2.08 (2H, br s), 1.62 (2H, br s).

### Example 86

### Synthesis of Compound 247

### Synthesis of Intermediate B160

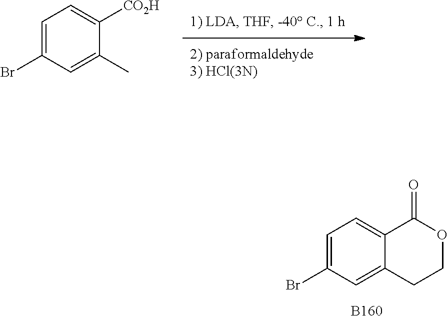

To a solution of LDA (29.06 mL, 2 mol/L, 2.50 equiv) in THF (80 mL) was added 4-bromo-2-methylbenzoic acid (5 g, 23.251 mmol, 1.00 equiv) in THF (20 mL) dropwise at −40° C. under N₂ atmosphere. The reaction mixture was stirred at −40° C. for 30 min. To the reaction mixture was added paraformaldehyde (2.79 g, 93.000 mmol, 4.00 equiv) in portions at 15° C. The reaction mixture was stirred for an additional 4 h at room temperature, then quenched with water (200 mL) at 0° C. The reaction mixture was acidified to pH 3 with HCl (aq), then extracted with ethyl acetate (3×50 mL). The combined organic layers were washed with brine (1×50 mL), dried over anhydrous Na₂SO₄, and filtered. After filtration, the filtrate was concentrated under reduced pressure to give a residue. The residue was purified by silica gel column chromatography, eluted with PE/EtOAc (3:1) to afford 6-bromo-3,4-dihydro-2-benzopyran-1-one (1.6g,30.31%) as a yellow solid. LCMS (ES, m/z): 227 [M+H]⁺.

### Synthesis of Intermediate B161

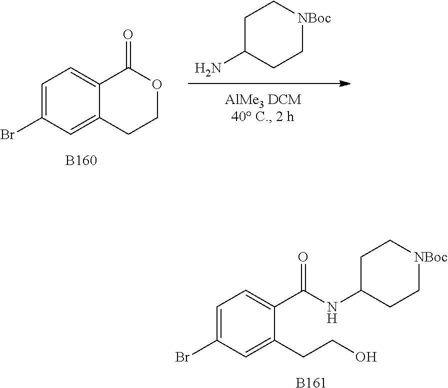 [SMILES: "B160" ; "B161" ; O=C1OCCc2cc(Br)ccc21 ; CC(C)(C)OC(=O)N1CCC(NC(=O)c2ccc(Br)cc2CCO)CC1 ; CC(C)(C)OC(=O)N1CCC(N)CC1]

To a stirred mixture of 6-bromo-3,4-dihydro-2-benzopyran-1-one (2.4 g, 10.570 mmol, 1.00 equiv) and tert-butyl 4-aminopiperidine-1-carboxylate (3.18 g, 15.855 mmol, 1.50 equiv) in DCM (50.00 mL) was added AlMe₃ in toluene (7.93 mL, 2 mol/L, 1.50 equiv) dropwise at 0° C. under nitrogen atmosphere. The resulting mixture was stirred for 2 h at 40° C. under nitrogen atmosphere, then quenched with water (100 mL) at 0° C. The reaction mixture was acidified to pH 3 with HCl (aq.), then extracted with ethyl acetate (3×50 mL). The combined organic layers were washed with brine (1×100 mL), dried over anhydrous Na₂SO₄, and filtered. After filtration, the filtrate was concentrated under reduced pressure to afford tert-butyl 4-[4-bromo-2-(2-hydroxyethyl) benzamido]piperidine-1-carboxylate (3.9g,86.34%) as a solid. LCMS (ES, m/z): 427 [M+H]⁺.

### Synthesis of Intermediate B162

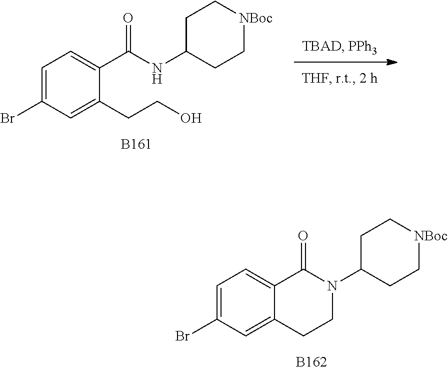 [SMILES: "B161" ; "B162" ; CC(C)(C)OC(=O)N1CCC(NC(=O)c2ccc(Br)cc2CCO)CC1 ; CC(C)(C)OC(=O)N1CCC(N2CCc3cc(Br)ccc3C2=O)CC1]

To a stirred solution of tert-butyl 4-[4-bromo-2-(2-hydroxyethyl) benzamido]piperidine-1-carboxylate (3.40 g, 7.956 mmol, 1.00 equiv) and PPh₃(4.17 g, 15.899 mmol, 2.00 equiv) in THF (120.00 mL) was added DTBAD (3.66 g, 15.912 mmol, 2.00 equiv) in THF (20 mL) dropwise at 0° C. under nitrogen atmosphere. The resulting mixture was stirred for 2 h at room temperature under nitrogen atmosphere, then concentrated under reduced pressure to give a residue. The residue was purified by silica gel chromatography, eluted with PE/EtOAc (3:1) to afford tert-butyl 4-(6-bromo-1-oxo-3,4-dihydroisoquinolin-2-yl) piperidine-1-carboxylate (2.8g,85.98%) as a solid. LCMS (ES, m/z): 409 [M+H]⁺.

### Synthesis of Intermediate B163

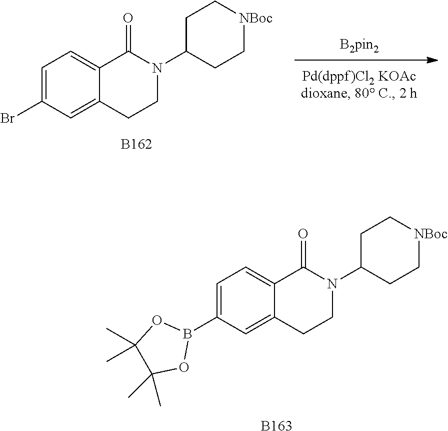

To a mixture of tert-butyl 4-(6-bromo-1-oxo-3,4-dihydroisoquinolin-2-yl) piperidine-1-carboxylate (2.50 g, 6.108 mmol, 1.00 equiv) and 4,4,5,5-tetramethyl-2-(tetramethyl-1,3,2-dioxaborolan-2-yl)-1,3,2-dioxaborolane (3.10 g, 12.208 mmol, 2.00 equiv) in dioxane (50.00 mL) was added KOAc (1.80 g, 18.341 mmol, 3.00 equiv) and Pd(dppf)Cl₂ CH₂Cl₂(0.50 g, 0.614 mmol, 0.10 equiv). The reaction mixture was stirred for 2 h at 80° C. under a nitrogen atmosphere, then concentrated under reduced pressure to give a residue. The residue was purified by silica gel chromatography, eluted with PE/EtOAc (3:1) to afford tert-butyl 4-[1-oxo-6-(4,4,5,5-tetramethyl-1,3,2-dioxaborolan-2-yl)-3,4-dihydroisoquinolin-2-yl]piperidine-1-carboxylate (2.5g,89.69%) as a solid. LCMS (ES, m/z): 457 [M+H]⁺.

### Synthesis of Intermediate B164

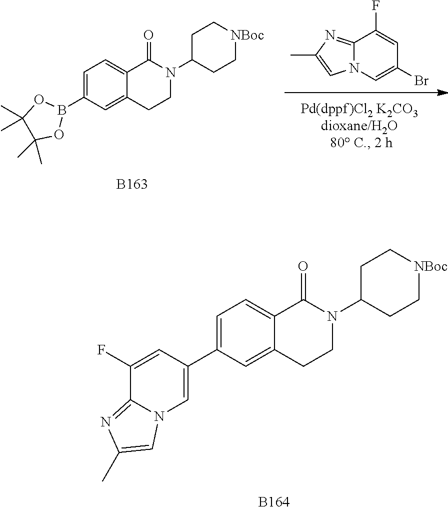 [SMILES: "B163" ; "B164" ; CC(C)(C)OC(=O)N1CCC(N2CCc3cc(B4OC(C)(C)C(C)(C)O4)ccc3C2=O)CC1 ; Cc1cn2cc(-c3ccc4c(c3)CCN(C3CCN(C(=O)OC(C)(C)C)CC3)C4=O)cc(F)c2n1 ; Cc1cn2cc(Br)cc(F)c2n1]

To a mixture of tert-butyl 4-[1-oxo-6-(4,4,5,5-tetramethyl-1,3,2-dioxaborolan-2-yl)-3,4-dihydroisoquinolin-2-yl]piperidine-1-carboxylate (120.00 mg, 0.263 mmol, 1.00 equiv) and 6-bromo-8-fluoro-2-methylimidazo[1,2-a] pyridine (66.25 mg, 0.289 mmol, 1.10 equiv) in dioxane (2.00 mL) and water (0.50 mL) was added K₂CO₃(109.02 mg, 0.789 mmol, 3.00 equiv) and Pd(dppf)Cl₂ CH₂Cl₂(10.71 mg, 0.013 mmol, 0.05 equiv). The reaction mixture was stirred for 2 h at 80° C. under a nitrogen atmosphere, then concentrated under reduced pressure to give a residue. The residue was purified by silica gel column chromatography, eluted with CH₂Cl₂/MeOH (15:1) to afford tert-butyl 4-(6-[8-fluoro-2-methylimidazo[1,2-a]pyridin-6-yl]-1-oxo-3,4-dihydroisoquinolin-2-yl) piperidine-1-carboxylate (100 mg,79.47%) as a solid. LCMS (ES, m/z): 479 [M+H]⁺.

### Synthesis of Compound 247

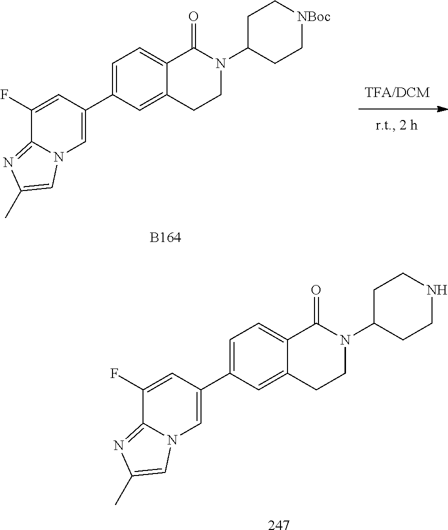 [SMILES: "B164" ; Cc1cn2cc(-c3ccc4c(c3)CCN(C3CCN(C(=O)OC(C)(C)C)CC3)C4=O)cc(F)c2n1 ; Cc1cn2cc(-c3ccc4c(c3)CCN(C3CCNCC3)C4=O)cc(F)c2n1 ; "247"]

A mixture of tert-butyl 4-(6-[8-fluoro-2-methylimidazo[1,2-a] pyridin-6-yl]-1-oxo-3,4-dihydroisoquinolin-2-yl) piperidine-1-carboxylate (100.00 mg, 0.209 mmol, 1.00 equiv) and TFA (0.20 mL) in DCM (1.00 mL) was stirred for 2 h at room temperature under nitrogen atmosphere, then concentrated under reduced pressure to give a residue. The residue was purified by Prep-HPLC (Column, XBridge Prep OBD C18 Column, 30×150 mm Sum; mobile phase, Water (10 mmol/L NH₄HCO₃) and acetonitrile; Gradient: 5% PhaseB up to 40% in 8 min) to afford 6-[8-fluoro-2-methylimidazo[1,2-a] pyridin-6-yl]-2-(piperidin-4-yl)-3,4-dihydroisoquinolin-1-one (36.5 mg, 46.16%) as a solid. LCMS (ES, m/z): 379 [M+H]⁺. ¹H NMR (400 MHz, DMSO-d6) δ 8.85 (d, J=1.6 Hz, 1H), 7.95 (d, J=8.0 Hz, 1H), 7.87-7.81 (m, 1H), 7.74-7.66 (m, 2H), 7.56 (dd, J=12.6, 1.5 Hz, 1H), 4.59-4.49 (m, 1H), 3.48 (t, J=6.5 Hz, 2H), 3.06-2.95 (m, 4H), 2.57 (d, J=12.0 Hz, 2H), 2.38 (s, 3H), 1.62 (tt, J=12.0, 6.0 Hz, 2H), 1.51 (d, J=11.6 Hz, 2H).

### Example 87

### Synthesis of Compound 239

### Synthesis of Compound 239

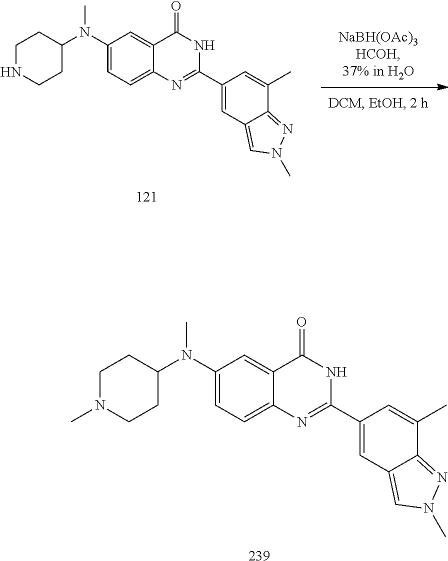

To a suspension of 2-(2,7-dimethyl-2H-indazol-5-yl)-6-(methyl(piperidin-4-yl)amino)quinazolin-4(3H)-one (28.0 mg, 0.070 mmol) in DCM (1.35 mL) and ethanol (0.43 mL) was added formaldehyde, 37% in water (25.9 uL, 0.348 mmol), followed by NaBH(OAc)₃ (88.5 mg, 0.417 mmol). The reaction mixture was stirred for 2 h. A solution of 10% NH₄OH was added dropwise and the resulting solution was concentrated in vacuo to give a residue. The residue was purified by flash chromatography on a silica gel column using a gradient of 10-30% methanol in DCM. Selected fractions were combined and concentrated in vacuo to give a residue. The residue was partitioned between a solution of 5% methanol in DCM (5 ml) and water (5 ml). To this suspension was added a saturated solution of NaHCO₃ (2.5 ml). The aqueous layer was extracted with a solution of 5% methanol in DCM (4×5 mL). The combined organic layers were washed with brine, dried over Na₂SO₄, filtered, and concentrated in vacuo to afford 2-(2,7-dimethyl-2H-indazol-5-yl)-6-(methyl(1-methylpiperidin-4-yl)amino)quinazolin-4(3H)-one (19 mg, 66%) as a solid. LCMS (ES, m/z): 417.3 [M+H]⁺. ¹H NMR (DMSO-d₆, 400 MHz): δ 12.13 (1H, s), 8.48 (1H, s), 8.38 (1H, s), 7.85 (1H, s), 7.60 (1H, d, J=9.0 Hz), 7.42 (1H, dd, J=9.2, 3.0 Hz), 7.28 (1H, d, J=3.0 Hz), 4.19 (3H, s), 3.67-3.75 (1H, m), 2.83-2.87 (5H, m), 2.55 (3H, s), 2.19 (3H, s), 2.06 (2H, t, J=11.5 Hz), 1.74-1.83 (2H, m), 1.61 (2H, d, J=11.8 Hz).

### Example 88

### Synthesis of Compound 306

### Synthesis of Compound 306

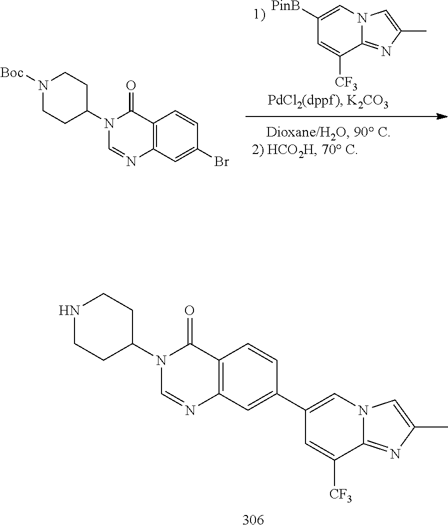

A mixture of tert-butyl 4-(7-bromo-4-oxoquinazolin-3(4H)-yl)piperidine-1-carboxylate (120 mg, 0.29 mmol), 2-methyl-6-(4,4,5,5-tetramethyl-1,3 ,2-dioxaborolan-2-yl)-8-(trifluoromethyl)imidazo[1,2-a]pyridine (134 mg, 0.41 mmol), PdCl₂(dppf) (21 mg, 0.029 mmol), and K₂CO₃ (203 mg, 1.47 mmol) was dissolved in dioxane (2.0 mL) and water (345 μL), then heated at 90° C. for 4 h under argon atmosphere. The reaction mixture was diluted with ethyl acetate (25 mL) and washed with saturated NaHCO₃ (20 mL) and brine (2×20 mL). The organic phase was then filtered under vacuum, dried over Na₂SO₄, filtered, and the filtrate concentrated in vacuo to give a residue. The residue was purified by flash chromatography on a silica gel column using a gradient of 70-100% ethyl acetate in hexane. Selected fractions were combined and evaporated under reduced pressure to yield a solid. To the resulting solid was added formic acid (5 mL) and the resulting mixture was stirred vigorously at 70° C. for 2 hours. The reaction mixture was concentrated under reduced pressure and diluted with DCM (20 mL). The organic phase was neutralized with 1 M NaOH (10 mL) and washed with brine (20 mL). The organic layer was dried over Na₂SO₄, filtered and the filtrate concentrated in vacuo to give a residue. The residue was purified by flash chromatography on a neutral alumina column using a gradient of 0-10% methanol in DCM to afford 7-(2-methyl-8-(trifluoromethyl)imidazo[1,2-a]pyridin-6-yl)-3-(piperidin-4-yl)quinazolin-4(3H)-one (28 mg, 26%) as a solid. LCMS (ES, m/z): 428.1 [M+H]⁺. ¹H NMR (CDCl₃, 300 MHz): δ 8.53 (1H, s), 8.44 (1H, d, J=8.3 Hz), 8.24 (1H, s), 7.91 (1H, s), 7.84 (1H, s), 7.72 (1H, d,=8.3 Hz), 7.57 (1H, s), 4.99 (1H, m), 3.33 (2H, d,=12.3 Hz), 2.90 (2H, t, J=11.9 Hz), 2.58 (3H, s), 1.81-2.03 (5H, br m).

### Example 89

### Synthesis of Compound 322

### Synthesis of Intermediate B165

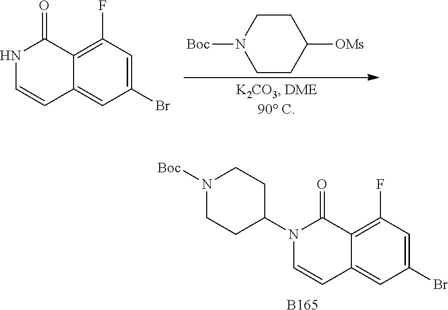 [SMILES: "B165" ; O=c1[nH]ccc2cc(Br)cc(F)c12 ; CC(C)(C)OC(=O)N1CCC(n2ccc3cc(Br)cc(F)c3c2=O)CC1 ; CC(C)(C)OC(=O)N1CCC(OS(C)(=O)=O)CC1]

A sealed tube was charged with 6-bromo-8-fluoroisoquinolin-1(2H)-one (450 mg, 1.86 mmol) and potassium carbonate (771 mg, 5.58 mmol). The mixture was dissolved in 1,2-dimethoxyethane (9 mL) and stirred vigorously for 20 minutes. To this suspension was added tert-butyl 4-((methylsulfonyl)oxy)piperidine-1-carboxylate (1.59 g, 5.58 mmol). The reaction mixture was stirred for 24 h at 100° C. The reaction mixture was concentrated in vacuo, taken up in CH₂Cl₂ (50 mL), and washed with saturated NaHCO₃ (20 mL). The organic layer was dried over anhydrous Na₂SO₄, filtered, and the filtrate concentrated under reduced pressure to give a residue. The residue was purified by flash chromatography on silica gel using a gradient of 0-100% ethyl acetate in hexane to afford tert-butyl 4-(6-bromo-8-fluoro-1-oxoisoquinolin-2(1H)-yl)piperidine-1-carboxylate (610 mg, 77%) as a solid. LCMS (ES, m/z): 447.0 [M+Na]⁺.

### Synthesis of Intermediate B166

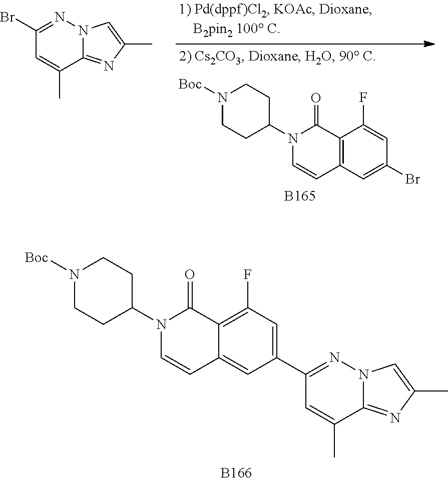 [SMILES: "B166" ; Cc1cn2nc(Br)cc(C)c2n1 ; "B165" ; Cc1cn2nc(-c3cc(F)c4c(=O)n(C5CCN(C(=O)OC(C)(C)C)CC5)ccc4c3)cc(C)c2n1 ; CC(C)(C)OC(=O)N1CCC(n2ccc3cc(Br)cc(F)c3c2=O)CC1]

A mixture of 6-bromo-2,8-dimethylimidazo[1,2-b]pyridazine (200 mg, 886 μmol), bis(pinacolato)diboron (225 mg, 886 μmol), Pd(dppf)Cl₂ (50 mg, 68.2 μmol), and potassium acetate (201 mg, 2.05 mmol) in dioxane (4.5 mL) was heated to 100° C. for 1 h. Then to the reaction mixture, a solution of tert-butyl 4-(6-bromo-8-fluoro-1-oxoisoquinolin-2(1H)-yl)piperidine-1-carboxylate (290 mg, 682 μmol) in dioxane (3.6 mL) was added, followed by cesium carbonate (667 mg, 2.05 mmol) and water (0.9 mL) under argon. The reaction mixture was heated at 90° C. for 2 h and then cooled to room temperature. The reaction mixture was filtered over celite using 10% methanol in DCM as eluent. The volatiles were evaporated under reduced pressure. Water (20 mL) and DCM (30 mL) were added, and the layers were separated. The aqueous layer was extracted with DCM (3×20 mL). The organic layers were combined, dried over Na₂SO₄, filtered, and the filtrate concentrated under reduced pressure to give a residue. The residue was purified by flash chromatography on silica gel using a gradient of 0-100% ethylacetate in DCM to afford tert-butyl 4-(6-(2,8-dimethylimidazo[1,2-b]pyridazin-6-yl)-8-fluoro-1-oxoisoquinolin-2(1H)-yl)piperidine-1-carboxylate (240 mg, 72%) as a solid. LCMS (ES, m/z): 492.2 [M+H]⁺.

### Synthesis of Compound 322

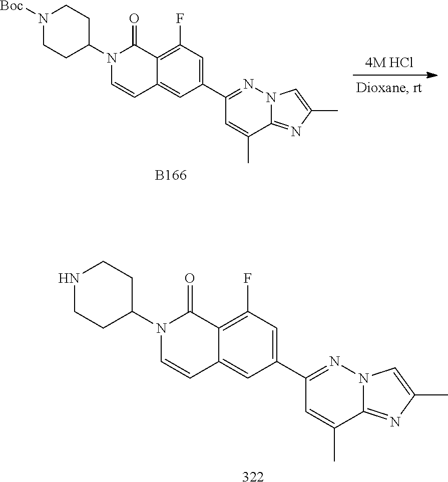 [SMILES: "B166" ; Cc1cn2nc(-c3cc(F)c4c(=O)n(C5CCN(C(=O)OC(C)(C)C)CC5)ccc4c3)cc(C)c2n1 ; Cc1cn2nc(-c3cc(F)c4c(=O)n(C5CCNCC5)ccc4c3)cc(C)c2n1 ; "322"]

To a solution of tert-butyl 4-(6-(2,8-dimethylimidazo[1,2-b]pyridazin-6-yl)-8-fluoro-1-oxoisoquinolin-2(1H)-yl)piperidine-1-carboxylate (240 mg, 488 μmol) in dioxane (4.9 mL) was added 4.0 M HCl in dioxane (9.76 mL, 39.1 mmol). The resulting mixture was stirred at room temperature for 1 hour. The reaction mixture was concentrated in vacuo, taken up in CH₂Cl₂ (2×30 mL), and washed with saturated NaHCO₃ (15 mL). The aqueous phase was extracted with a DCM (2×20 mL). The organic layers were combined, dried over Na₂SO₄, filtered, and the filtrate concentrated in vacuo to give a residue. The residue was purified on a silica gel cartridge using a gradient of MeOH/NH₄OH (9:1) from 0-20% in CH₂Cl₂ to afford 6-(2,8-dimethylimidazo[1,2-b]pyridazin-6-yl)-8-fluoro-2-(piperidin-4-yl)isoquinolin-1(2H)-one (163 mg, 85%) as a solid. LCMS (ES, m/z): 392.2 [M+H]⁺. ¹H NMR (CHCl₃-d, 400 MHz): δ_{H} 7.81 (2H, d, J=10.9 Hz) 7.72 (1H, d, J=12.6 Hz), 7.30 (1H, s), 7.24 (1H, d, J=10.9 Hz), 6.59 (1H, d, J=7.5 Hz), 5.15-5.09 (1H, m), 3.26 (2H, d, J=12.2 Hz), 2.87 (2H, t, J=11.9 Hz), 2.73 (3H, s), 2.55 (3H, s), 1.94 (2H, d, J=12.0 Hz), 1.78 (2H, qd, J=12.1, 4.0 Hz).

### Example 90

### Exemplary Splicing Assay for Monitoring Expression Levels of Splice Variants

Compounds described herein were used to modulate RNA transcript abundance in cells. The expression of a target mRNA was measured by detecting the formation of an exon-exon junction in the canonical transcript (CJ). A compound mediated exon-inclusion event was detected by observing an increase in formation of a new junction with an alternative exon (AJ). Real-time qPCR assays were used to detect these splicing switches and interrogate the potency of various compounds towards different target genes. A high-throughput real time quantitative PCR (RT-qPCR) assay was developed to measure these two isoforms of the mRNA (CJ and AJ) for an exemplary gene, HTT, together with a control housekeeping gene, GAPDH or GUSB or PPIA, used for normalization. Briefly, the A673 or K562 cell line was treated with various compounds described herein (e.g., compounds of Formula (I)). After treatment, the levels of the HTT mRNA targets were determined from each sample of cell lysate by cDNA synthesis followed by qPCR.

### Materials:

Cells-to-C_{T} 1-step kit: ThermoFisher A25602, Cells-to-CT lysis reagent: ThermoFisher 4391851C, TaqMan™ Fast Virus 1-Step Master Mix: ThermoFisher 4444436
GAPDH: VIC-PL, ThermoFisher 4326317E (Assay: Hs99999905_m1)—used for K562/suspension cell lines
GUSB: VIC-PL, ThermoFisher 4326320E (Assay: Hs99999908_m1)—used for K562/suspension cell lines
PPIA: VIC-PL, ThermoFisher 4326316E (Assay: Hs99999904_m1) — used for A673/adherent cell lines

Probe/primer sequences
Canonical junction (CJ)
HTT Primer 1: TCCTCCTGAGAAAGAGAAGGAC
HTT Primer 2: GCCTGGAGATCCAGACTCA
HTT CY5-Probe: /5Cy5/TGGCAACCCTTGAGGCCCTGTCCT/
3IAbRQSp/
Alternative junction (AJ)
HTT Primer 1: TCCTGAGAAAGAGAAGGACATTG
HTT Primer 2: CTGTGGGCTCCTGTAGAAATC
HTT FAM-Probe: /56-FAM/TGGCAACCC/ZEN/TTGAGAGGCAAGC
CCT/3IABkFQ/

### Description

The A673 cell line was cultured in DMEM with 10% FBS. Cells were diluted with full growth media and plated in a 96-well plate (15,000 cells in 100 ul media per well). The plate was incubated at 37° C. with 5% CO₂ for 24 hours to allow cells to adhere. An 11-point 3-fold serial dilution of the compounds was made in DMSO then diluted in media in an intermediate plate. Compounds were transferred from the intermediate plate to the cell plate with the top dose at a final concentration of 10 uM in the well. Final DMSO concentration was kept at or below 0.25%. The cell plate was returned to the incubator at 37° C. with 5% CO₂ for an additional 24 hours.

The K562 cell line was cultured in IMDM with 10% FBS. For K562, cells were diluted with full growth media and plated in either a 96-well plate (50,000 cells in 50 uL media per well) or a 384-well plate (8,000-40,000 cells in 45 uL media per well). An 11-point 3-fold serial dilution of the compounds were made in DMSO then diluted in media in an intermediate plate. Compound was transferred from the intermediate plate to the cell plate with the top dose at a final concentration of 10 uM in the well. Final DMSO concentration was kept at or below 0.25%. Final volume was 100 uL for 96-well plate and 50 uL for 384-well plate. The cell plate was then placed in an incubator at 37° C. with 5% CO2 for 24 hours.

The cells were then gently washed with 50 uL-100 uL cold PBS before proceeding to addition of lysis buffer. 30 uL-50 uL of room temperature lysis buffer with DNAse I (and optionally RNAsin) was added to each well. Cells were shaken/mixed thoroughly at room temperature for 5-10 minutes for lysis to take place and then 3 uL-5 uL of room temperature stop solution was added and wells were shaken/mixed again. After 2-5 minutes, the cell lysate plate was transferred to ice for RT-qPCR reaction setup. The lysates could also be frozen at −80° C. for later use.

In some cases, a direct lysis buffer was used. An appropriate volume of 3× lysis buffer (10 mM Tris, 150 mM NaCl, 1.5%-2.5% Igepal and 0.1-1 U/uL RNAsin, pH 7.4) was directly added to either K562 or A673 cells in media and mixed by pipetting 3 times. The plates were then incubated at room temperature with shaking/rocking for 20-50 minutes to allow for lysis to take place. After this time, the cell lysate plate was transferred to ice to set up for the RT-qPCR reactions. The lysates could also be frozen at −80° C. for later use.

To set up 10 uL RT-qPCR reactions, cell lysates were transferred to 384-well qPCR plates containing the master mix according to the table below. The plates were sealed, gently vortexed, and spun down before the run. The volumes were adjusted accordingly in some instances where the reaction was carried in 20 uL. The table below summarizes the components of the RT-qPCR reactions:

| Component | 1X |
|---|---|
| Taqman 1-step RT-qPCR mix (4X) | 2.5 |
| 20X AJ Primers + Probe (FAM) | 0.5 |
| 20X CJ Primers + Probe (CY5) | 0.5 |
| 20X PPIA Control (VIC) | 0.5 |
| Cell lysate (1X) | 1-2 |
| H₂O | 4-5 |
| Total volume | 10 |

The RT-qPCR reaction was performed using a QuantStudio (ThermoFisher) under the following fast cycling conditions. All samples and standards were analyzed at least in duplicate. In some instances, bulk room temperature (RT) step of 5-10 minutes was completed for all plates before proceeding with qPCR. The table below summarizes the PCR cycle:

| Step | # cycles | Temp. | Time |
|---|---|---|---|
| RT step | 1 | 50° C. | 5 min |
| RT inactivation/ | 1 | 95° C. | 20 sec |
| initial denaturation | | | |
| Amplification | 40 | 95° C. | 3 sec |
| | | 60° C. | 30 sec |

The data analysis was performed by first determining the ΔCt vs the housekeeper gene. This ΔCt was then normalized against the DMSO control (ΔΔCt) and converted to RQ (relative quantification) using the 2^(−ΔΔCt) equation. The RQ were then converted to a percentage response by arbitrarily setting an assay window of 3.5 ΔCt for HTT-CJ and an assay window of 9 ΔCt for HTT-AJ. These assay windows correspond to the maximal modulation observed at high concentration of the most active compounds. The percentage response was then fitted to the 4 parametric logistic equation to evaluate the concentration dependence of compound treatment. The increase in AJ mRNA is reported as AC₅₀ (compound concentration having 50% response in AJ increase) while the decrease in CJ mRNA levels is reported as IC₅₀ (compound concentration having 50% response in CJ decrease).

A summary of these results is illustrated in Table 6, wherein “A” represents an AC₅₀/IC₅₀ of less than 100 nM; “B” represents an AC₅₀/IC₅₀of between 100 nM and 1 μM; and “C” represents an AC₅₀/IC₅₀ of between 1 μM and 10 μM; and “D” represents an AC₅₀/IC₅₀ of greater than 10 μM.

TABLE 6
Modulation of RNA Splicing by Exemplary Compounds

| | HTT AJ | HTT CJ |
|---|---|---|
| Compound | AC₅₀ | AC₅₀ |
| No. | (nM) | (nM) |
| 108 | D | D |
| 152 | D | D |
| 153 | D | D |
| 156 | C | C |
| 157 | D | D |
| 158 | D | D |
| 159 | D | D |
| 160 | D | D |
| 161 | D | D |
| 162 | D | D |
| 163 | D | D |
| 165 | D | D |
| 166 | C | C |
| 167 | D | D |
| 172 | D | D |
| 173 | D | D |
| 174 | B | B |
| 175 | C | D |
| 176 | C | C |
| 177 | D | D |
| 178 | D | D |
| 179 | D | D |
| 180 | D | D |
| 181 | B | B |
| 182 | C | C |
| 185 | C | B |
| 186 | A | A |
| 188 | C | C |
| 190 | B | B |
| 191 | D | D |
| 192 | D | D |
| 203 | D | D |
| 204 | D | D |
| 205 | B | B |
| 206 | B | B |
| 207 | C | C |
| 208 | C | D |
| 209 | B | C |
| 210 | C | C |
| 215 | C | C |
| 216 | C | C |
| 217 | C | C |
| 218 | C | C |
| 219 | C | C |
| 220 | D | D |
| 221 | D | D |
| 222 | C | C |
| 223 | C | C |
| 224 | D | D |
| 225 | D | D |
| 226 | D | D |
| 227 | C | C |
| 228 | B | B |
| 229 | D | D |
| 230 | A | A |
| 231 | B | B |
| 232 | A | A |
| 233 | B | B |
| 234 | C | C |
| 235 | D | D |
| 236 | B | B |
| 237 | B | B |
| 238 | D | D |
| 239 | D | D |
| 241 | C | C |
| 242 | A | A |
| 243 | A | A |
| 244 | D | D |
| 245 | B | B |
| 246 | C | C |
| 247 | C | C |
| 248 | B | A |
| 249 | C | C |
| 250 | A | A |
| 251 | B | B |
| 252 | D | D |
| 253 | C | C |
| 254 | C | C |
| 255 | C | C |
| 256 | B | B |
| 257 | B | B |
| 258 | D | C |
| 259 | B | B |
| 260 | B | B |
| 261 | D | D |
| 262 | C | C |
| 263 | D | D |
| 264 | C | C |
| 265 | A | A |
| 266 | A | A |
| 267 | B | C |
| 268 | C | C |
| 269 | D | D |
| 270 | C | C |
| 271 | C | C |
| 272 | B | A |
| 273 | B | A |
| 274 | A | A |
| 276 | A | A |
| 277 | B | B |
| 278 | A | A |
| 279 | B | B |
| 280 | B | B |
| 281 | C | C |
| 282 | D | D |
| 283 | B | B |
| 284 | D | D |
| 285 | D | C |
| 286 | C | C |
| 287 | B | B |
| 288 | C | C |
| 290 | B | B |
| 297 | C | C |
| 298 | B | B |
| 299 | D | D |
| 300 | C | B |
| 301 | C | C |
| 302 | C | C |
| 303 | C | C |
| 304 | C | C |
| 305 | C | B |
| 308 | C | C |
| 313 | D | D |

Additional studies were carried out for a larger panel of genes using the protocol provided above. The junction between flanking upstream and downstream exons was used to design canonical junction qPCR assays. At least one of the forward primer, reverse primer or the CY5-labeled 5′ nuclease probe (with 3′ quencher such as ZEN/Iowa Black FQ) was designed to overlap with the exon junction to capture the CJ mRNA transcript. BLAST was used to confirm the specificity of the probeset and parameters such as melting temperature, GC content, amplicon size, and primer dimer formation are considered during their design. Data for the decrease in CJ mRNA levels for three exemplary genes (HTT, SMN2, and Target C) analyzed in this panel are reported as IC₅₀ (compound concentration having 50% response in CJ decrease).

A summary of the results from the panel is illustrated in Table 7, wherein “A” represents an IC₅₀ of less than 100 nM; “B” represents an IC₅₀ of between 100 nM and 1 μM; and “C” represents an IC₅₀ of between 1 μM and 10 μM; and “D” represents an IC₅₀ of greater than 10 μM.

TABLE 7
Modulation of RNA Splicing by Exemplary Compounds

| Compound No. | HTT | SMN2 | Target C |
|---|---|---|---|
| 108 | D | C | C |
| 152 | D | D | D |
| 153 | D | C | — |
| 156 | C | B | — |
| 157 | D | C | — |
| 158 | D | D | — |
| 159 | D | D | — |
| 160 | D | D | D |
| 161 | D | D | D |
| 162 | D | D | — |
| 163 | D | D | D |
| 165 | D | B | — |
| 166 | C | C | C |
| 167 | D | D | D |
| 172 | D | D | D |
| 173 | D | C | — |
| 174 | B | A | — |
| 175 | C | C | D |
| 176 | C | B | C |
| 177 | D | D | C |
| 178 | D | D | D |
| 179 | D | C | D |
| 180 | D | D | C |
| 181 | B | A | B |
| 182 | C | A | C |
| 190 | B | B | B |
| 191 | D | D | D |
| 192 | D | D | D |
| 203 | D | D | D |
| 204 | D | D | D |
| 205 | B | A | B |
| 206 | B | A | B |
| 207 | C | B | C |
| 208 | D | B | D |
| 209 | C | A | C |
| 210 | C | B | C |
| 215 | C | A | C |
| 216 | C | A | C |
| 217 | C | B | D |
| 218 | C | C | C |
| 219 | C | A | C |
| 220 | D | D | D |
| 221 | D | B | D |
| 222 | C | B | C |
| 223 | C | B | D |
| 224 | D | B | D |
| 225 | D | D | D |
| 226 | D | B | D |
| 227 | C | B | D |
| 228 | B | B | B |
| 229 | D | D | D |
| 230 | A | A | A |
| 231 | B | A | C |
| 232 | A | A | B |
| 233 | B | A | C |
| 234 | C | A | D |
| 235 | D | B | D |
| 236 | B | A | B |
| 237 | B | A | C |
| 238 | D | D | D |
| 239 | D | C | D |
| 241 | C | C | C |
| 242 | A | A | B |
| 243 | A | A | A |
| 244 | D | B | D |
| 245 | B | A | B |
| 246 | C | C | C |
| 247 | C | A | C |
| 248 | A | A | B |
| 249 | C | C | C |
| 250 | A | A | B |
| 251 | B | A | B |
| 252 | D | B | D |
| 253 | C | B | C |
| 254 | C | C | C |
| 255 | C | B | D |
| 256 | B | A | D |
| 257 | B | A | C |
| 259 | B | B | B |
| 260 | B | B | C |
| 261 | D | C | D |
| 262 | C | A | C |
| 263 | D | D | D |
| 264 | C | B | D |
| 265 | A | A | B |
| 266 | A | A | B |
| 267 | C | A | C |
| 268 | C | A | D |
| 269 | D | C | D |
| 270 | C | B | D |
| 271 | C | B | C |
| 272 | A | A | B |
| 273 | A | — | B |
| 274 | A | A | A |
| 275 | D | — | D |
| 276 | A | A | A |
| 277 | B | A | C |
| 278 | A | A | B |
| 279 | B | A | B |
| 280 | B | A | C |
| 281 | C | B | C |
| 282 | D | C | D |
| 283 | B | A | B |
| 284 | D | C | D |
| 285 | C | A | D |
| 286 | C | B | C |
| 287 | B | A | B |
| 288 | C | C | C |
| 290 | B | B | B |
| 297 | C | A | C |
| 298 | B | A | C |
| 299 | D | D | D |
| 300 | B | A | C |
| 301 | C | C | C |
| 302 | C | B | C |
| 303 | C | B | C |
| 304 | B | A | C |
| 305 | B | — | B |
| 308 | C | B | C |
| 313 | D | B | D |

### EQUIVALENTS AND SCOPE

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, Figures, or Examples but rather is as set forth in the appended claims. Those of ordinary skill in the art will appreciate that various changes and modifications to this description may be made without departing from the spirit or scope of the present invention, as defined in the following claims.

1. A compound of Formula (V): 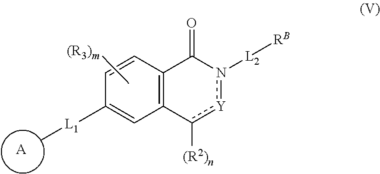
or a pharmaceutically acceptable salt, solvate, hydrate, tautomer, or stereoisomer thereof, wherein:
A is cycloalkyl, heterocyclyl, aryl, or heteroaryl, each of which is optionally substituted with one or more R¹;
R^{B} is B, C₁-C₆-alkyl, or C₁-C₆-heteroalkyl, wherein alkyl and heteroalkyl are substituted by one or more R¹⁰;
B is cycloalkyl, heterocyclyl, aryl, or heteroaryl, each of which is optionally substituted with one or more R¹;
each of which is optionally substituted with one or more R¹; each of L¹ and L² is independently absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁷;
Y is N, C(R^{6a}), or C(R^{6a})(R^{6b}), _{w}hₑrein the dashed lines in the ring comprising Y may be single or double bonds as valency permits;
each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R9^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁵; or
two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵;
each R² is independently hydrogen or C₁-C₆-alkyl;
R³ is C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —C(O)R^{D}, or —C(O)OR^{D};
R⁴ is hydrogen, C₁-C₆-alkyl, or C₁-C₆-haloalkyl;
each R⁵ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁷;
R^{6a} and R^{6b} is independently hydrogen, C₁-C₆-alkyl, C₁-C₆-heteroalkyl, C₁-C₆-haloalkyl, or halo;
each R⁷ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A};
each R^{A}is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D};
each R^{B} and R^{C} is independently hydrogen, C₁-C₆ alkyl, C₁-C₆ heteroalkyl, cycloalkyl, heterocyclyl, —OR^{A}; or R^{B} and R^{C} together with the atom to which they are attached form a 3-7-membered heterocyclyl ring optionally substituted with one or more R⁹;
each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl;
each R⁹ and R¹⁰ is independently C₁-C₆-alkyl or halo;
n is 0, 1, or 2;
m is 0, 1, 2, or 3; and
x is 0, 1, or 2.

2. The compound of claim 1, wherein A is a heterocyclyl or heteroaryl.

3. The compound of any one of claims 1-2, wherein A is a nitrogen-containing heterocyclyl or nitrogen-containing heteroaryl.

4. The compound of any one of claims 1-3, The compound of any one of the preceding claims, wherein A is selected from 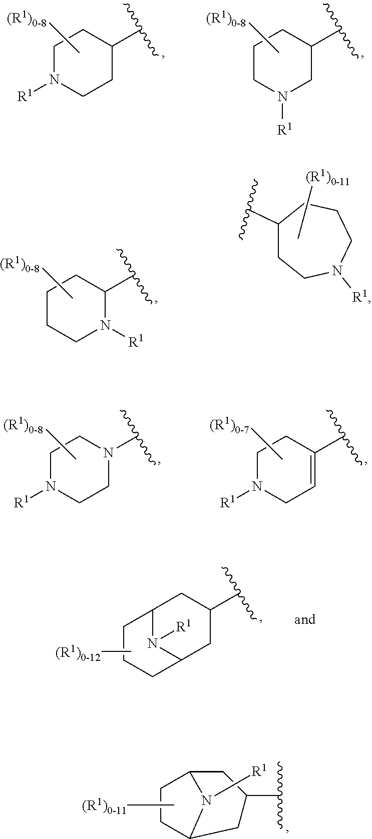
wherein R¹ is as described in claim 1.

5. The compound of any one of claims 1-4, wherein A is selected from

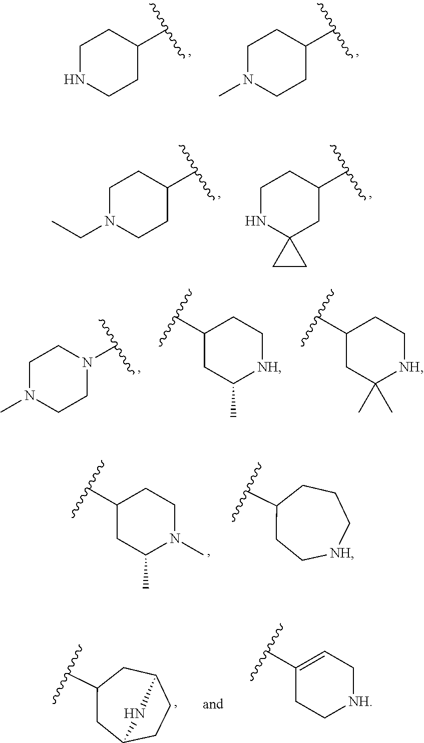

6. The compound of any one of claims 1-3, wherein A selected from 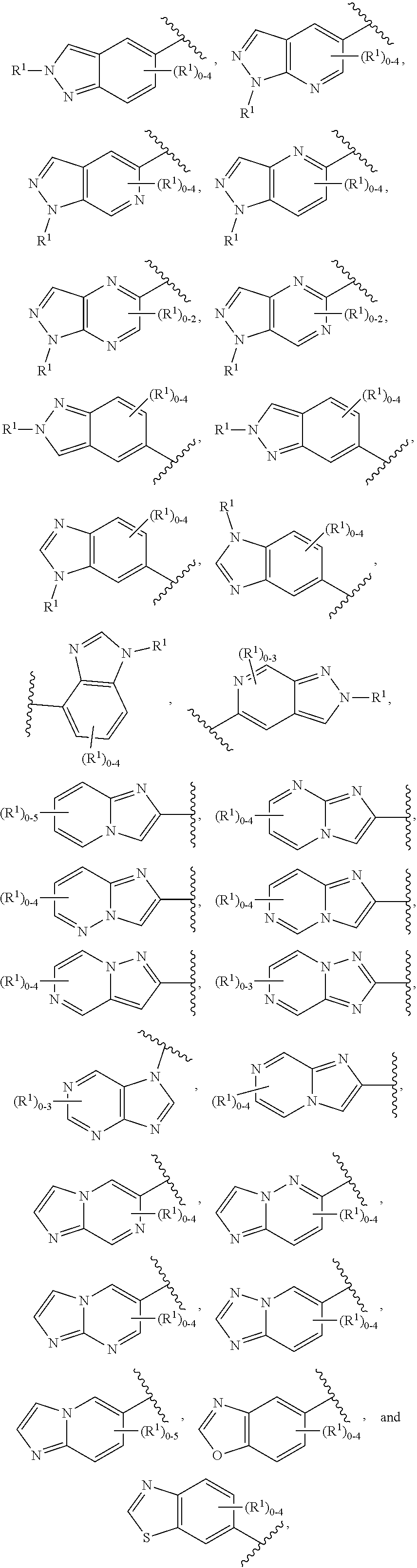
wherein R¹ is as described in claim 1.

7. The compound of claim 6, wherein A is selected from

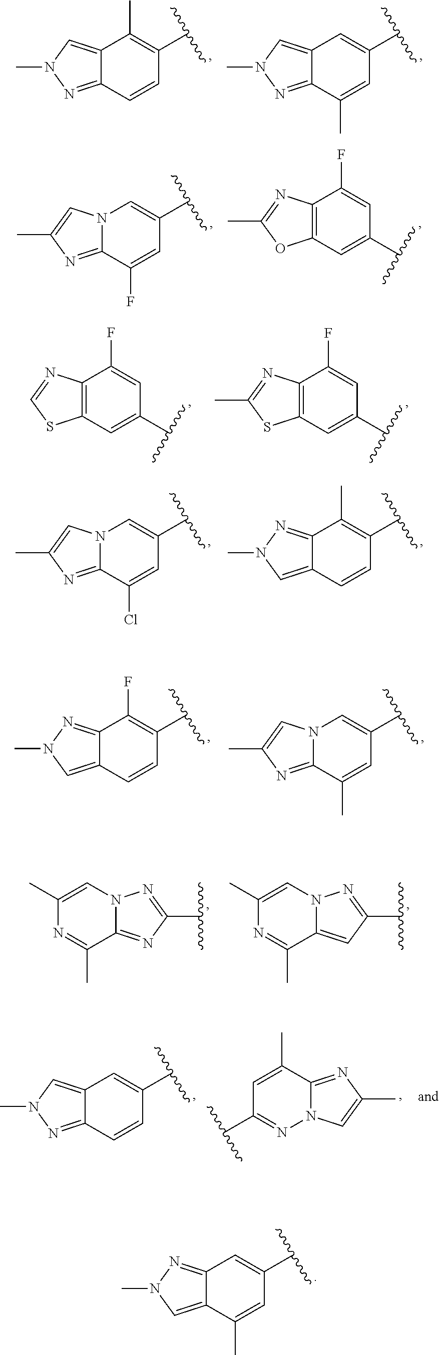

8. The compound of any one of claims 1-7, wherein B is a heteroaryl or heterocyclyl.

9. The compound of any one of claims 1-8, wherein B is a nitrogen-containing heteroaryl or nitrogen-containing heterocyclyl.

10. The compound of any one of claims 1-9, wherein B selected from selected from 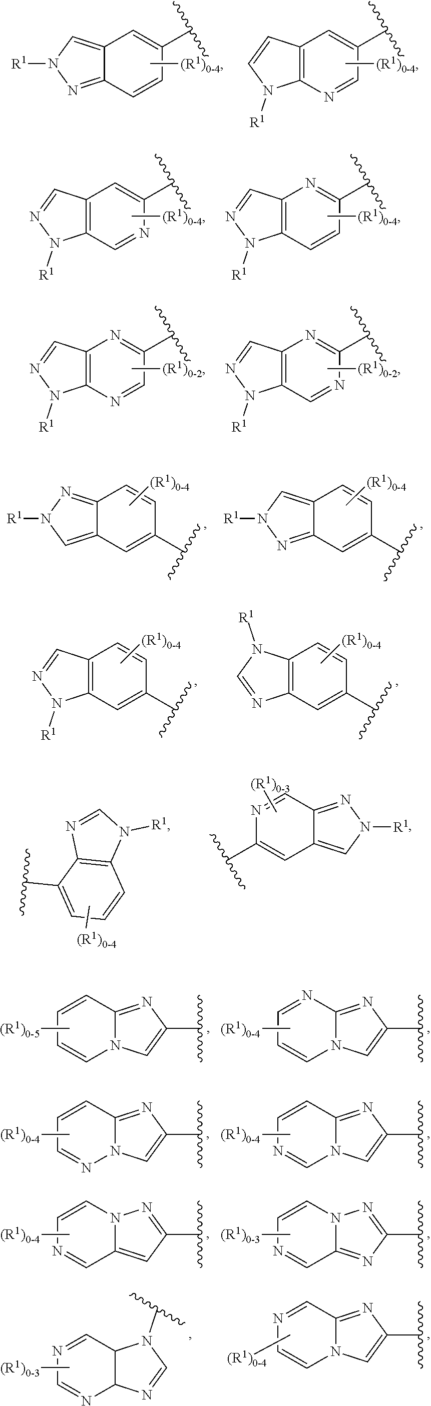 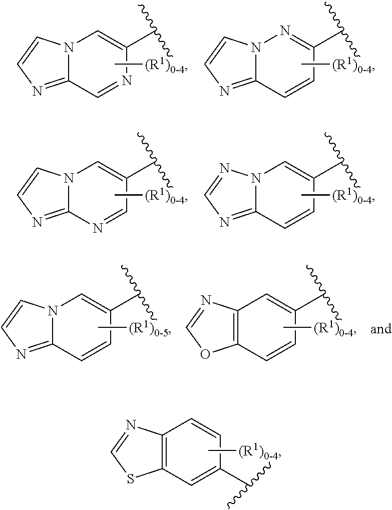
wherein R¹ is as described in claim 1.

11. The compound of any one of claims 49-58, wherein B is selected from

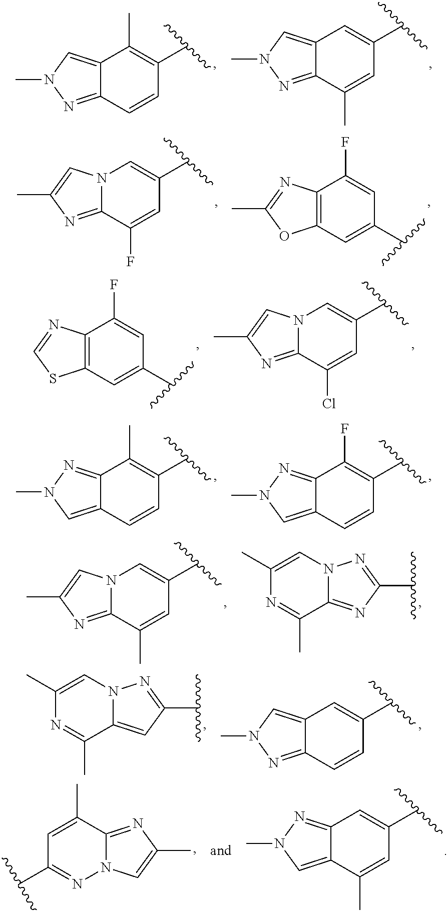

12. The compound of any one of claims 1-10, wherein B is selected from 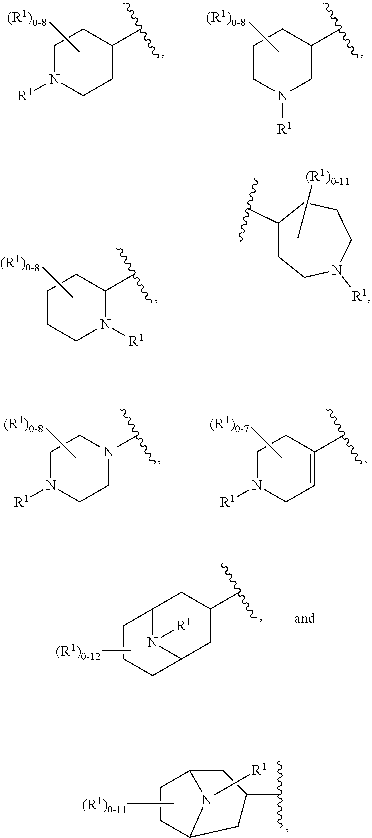
wherein R¹ is as described in claim 1.

13. The compound of claim 12, wherein B is selected from

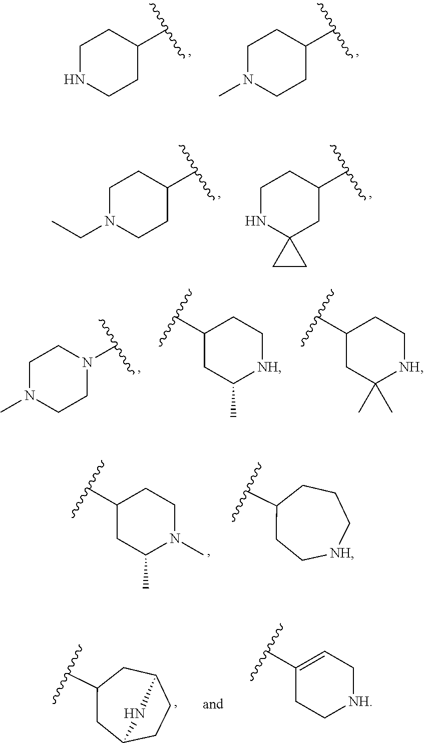

14. The compound of any one of claims 1-13, wherein each of L¹ and L² is independently absent.

15. The compound of any one of claims 1-15, wherein Y is C(R^{6a}) (e.g., CH) or N.

16. The compound of any one of claims 1-15, wherein R² is hydrogen.

17. The compound of any one of claims 1-16, wherein n is 1 or 2.

18. The compound of any one of claims 1-17, wherein the compound of Formula (V) is a compound of Formula (V-a): 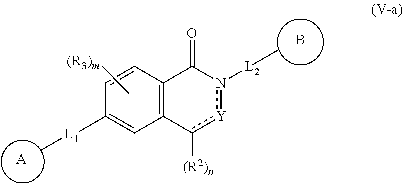
or a pharmaceutically acceptable salt, solvate, hydrate, tautomer, or stereoisomer thereof, wherein A, B, L¹, L², Y, R², R³, m, n, and subvariables thereof are as defined in claim 1.

19. The compound of any one of claims 1-18, wherein the compound of Formula (V) is a compound of Formula (V-b): 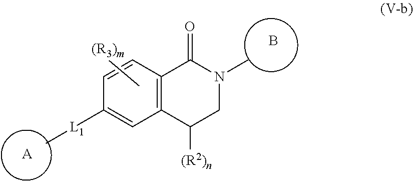
or a pharmaceutically acceptable salt, solvate, hydrate, tautomer, or stereoisomer thereof, wherein A, B, L¹, R², R³, and subvariables thereof are as defined in claim 1.

20. The compound of any one of claims 1-19, wherein the compound of Formula (V) is a compound of Formula (V-c): 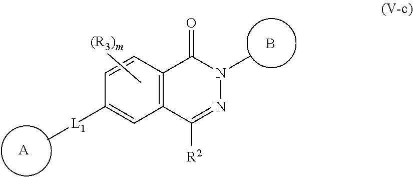
or a pharmaceutically acceptable salt, solvate, hydrate, tautomer, or stereoisomer thereof, wherein A, B, L¹, R², R³, and subvariables thereof are as defined in claim 1.

21. The compound of any one of claims 1-20, wherein the compound of Formula (V) is a compound of Formula (V-d): 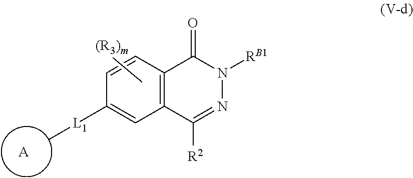
or a pharmaceutically acceptable salt, solvate, hydrate, tautomer, or stereoisomer thereof, wherein A, B, L¹, R², R³, and subvariables thereof are as defined in claim 1.

22. The compound of any one of claims 1-22, wherein the compound of Formula (V) is a compound listed in Table 5 or a pharmaceutically acceptable salt, solvate, hydrate, tautomer, or stereoisomer thereof.

23. A compound of Formula (I): 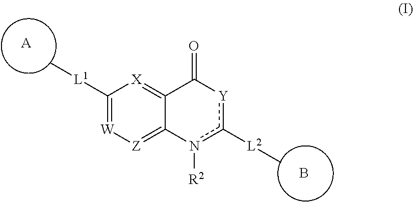
or a pharmaceutically acceptable salt, solvate, hydrate, tautomer, or stereoisomer thereof, wherein:
A and B are each independently cycloalkyl, heterocyclyl, aryl, or heteroaryl, each of which is optionally substituted with one or more R¹;
each of L1 and L2 is independently absent, C₁-C₆-alkylene, C₁-C₆-heteroalkylene, —O—, —C(O)—, —N(R⁸)—, —N(R⁸)C(O)—, or —C(O)N(R⁸)—, wherein each alkylene and heteroalkylene is optionally substituted with one or more R⁹;
each of W, X, and Z is independently C(R³) or N;
Y is N, N(R^{4a}), C(R^{4b}), or C(R^{4b})(R^{4c}), wherein the dashed lines in the ring comprising Y may be single or double bonds as valency permits;
each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, NR^{B}C(O)R^{D} —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁵; or
two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵;
R² is absent, hydrogen, or C₁-C₆-alkyl;
R³ is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —C(O)R^{D}, or —C(O)OR^{D};
R^{4a} is hydrogen, C₁-C₆-alkyl, C₁-C₆-heteroalkyl, or C₁-C₆-haloalkyl;
each of R^{4b} and R^{4c} is independently hydrogen, C₁-C₆-alkyl, C₁-C₆-heteroalkyl, C₁-C₆-haloalkyl, halo, or —OR^{A};
each R⁵ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C} , —NR^{B}C(O)R^{D}, —NO₂, —C(O)NR^{B}R^{C}, —C(O)R^{D} C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁶;
each R⁶ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A};
each R⁸ is independently hydrogen, C₁-C₆-alkyl, or C₁-C₆-haloalkyl;
each R⁹ is independently C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D};
each R^{A}is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D};
each R^{B} and R^{C} is independently hydrogen, C₁-C₆ alkyl, C₁-C₆ heteroalkyl, cycloalkyl, heterocyclyl, —OR^{A}; or
R^{B} and R^{C} together with the atom to which they are attached form a 3-7-membered heterocyclyl ring optionally substituted with one or more R¹⁰,
each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl;
each R¹⁰ is independently C₁-C₆-alkyl or halo; and
x is 0, 1, or 2.

24. The compound of claim 23, wherein A is heterocyclyl or heteroaryl.

25. The compound of any one of claims 23-24, wherein A is a nitrogen-containing heterocyclyl or nitrogen-containing heteroaryl.

26. The compound of any one of claims 23-25, wherein A is selected from 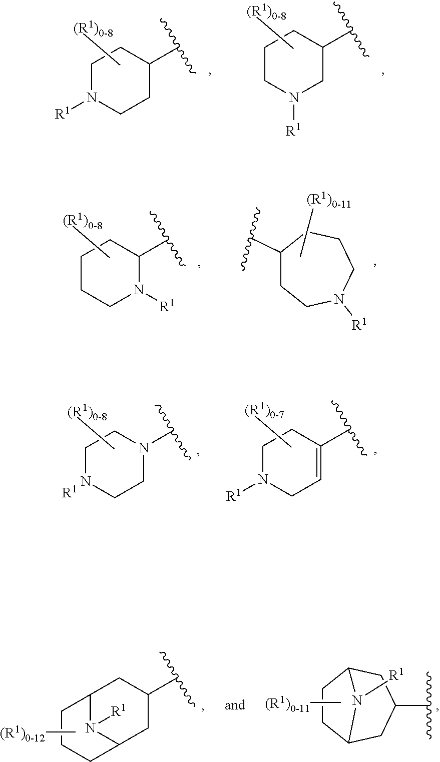
wherein R¹ is as described in claim 23.

27. The compound of claim 26, wherein A is selected from

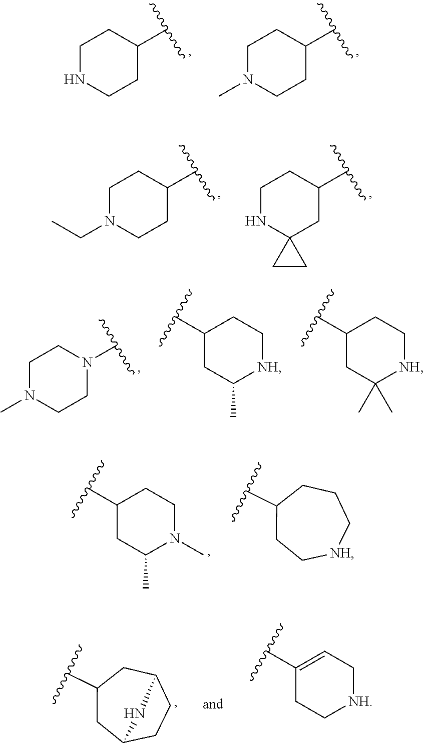

28. The compound of any one of claims 23-25, wherein A selected from 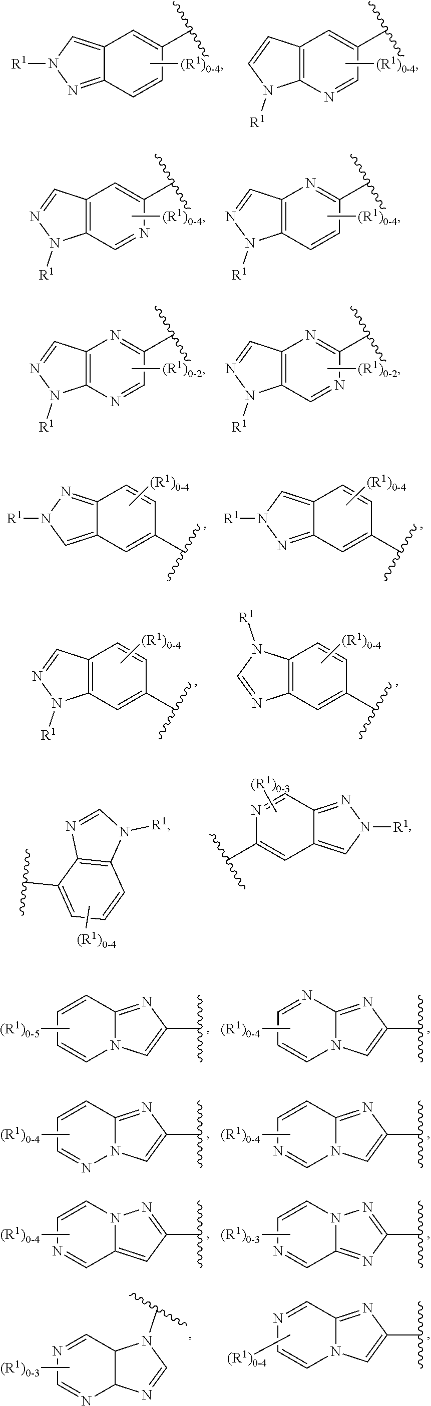 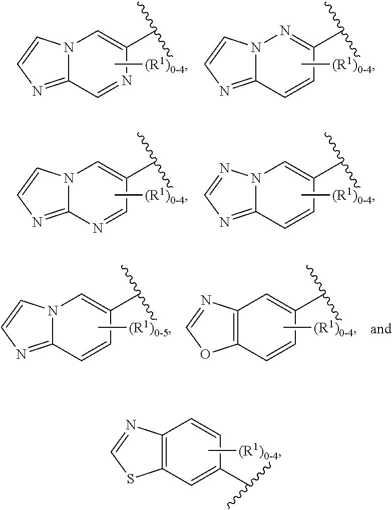
wherein R¹ is as described in claim 23.

29. The compound of claim 28, wherein A is selected from

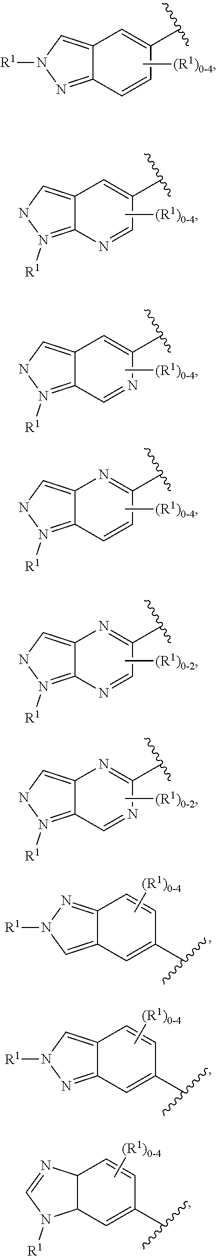

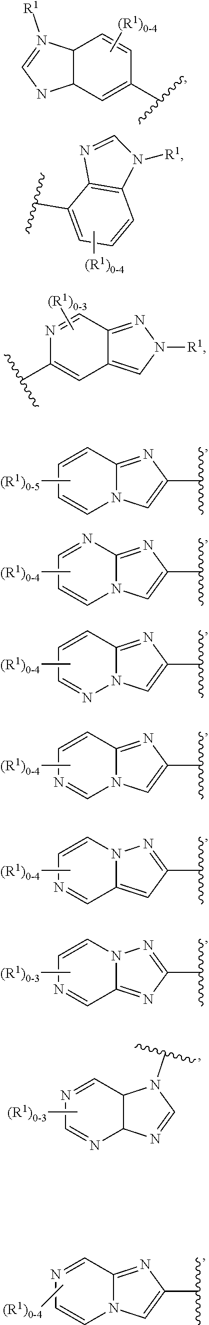

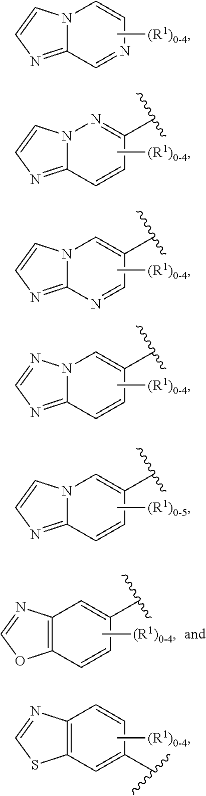

30. The compound of any one of claims 23-29, wherein B is a heterocyclyl or heteroaryl.

31. The compound of any one of claims 23-30, wherein B is a nitrogen-containing heteroaryl or nitrogen-containing heterocyclyl.

32. The compound of any one of claims 23-31, wherein B selected from 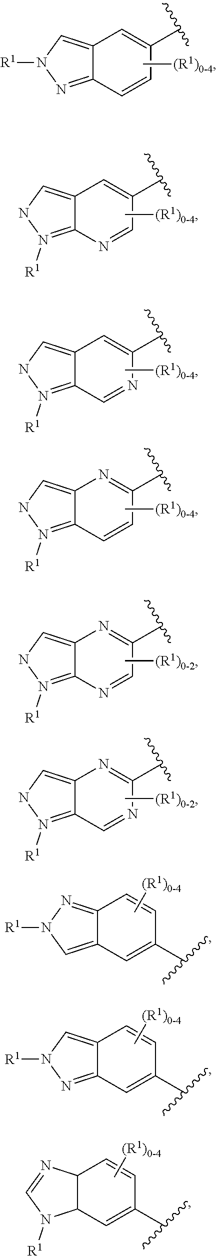 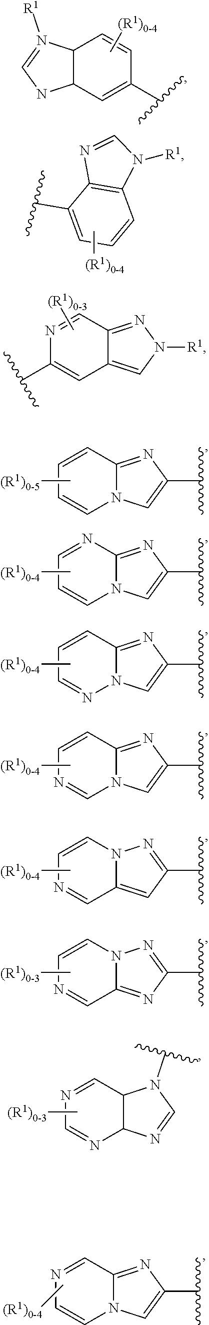 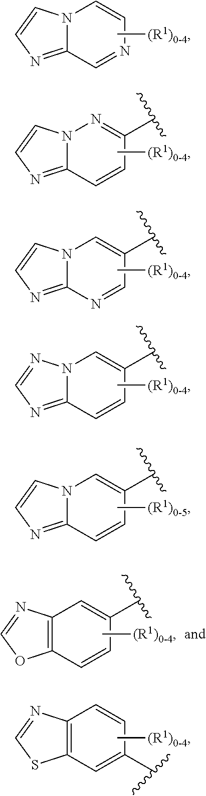
wherein R¹ is as described in claim 23.

33. The compound of claim 32, wherein B is selected from

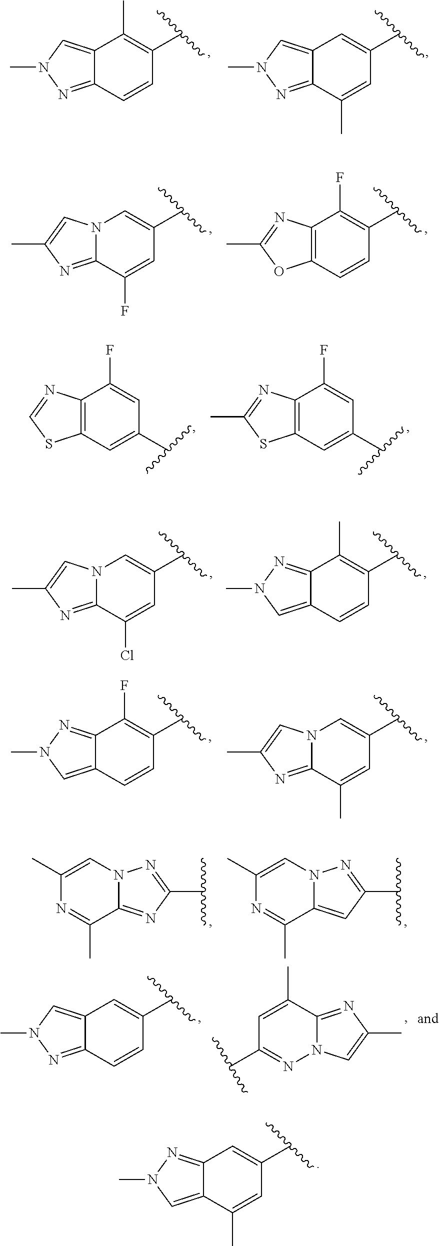

34. The compound of any one of claims 23-31, wherein B is selected from 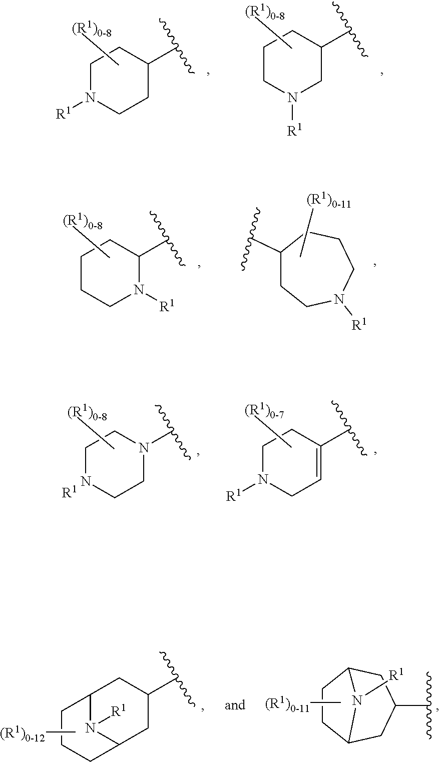
wherein R¹ is as described in claim 23.

35. The compound of claim 34, wherein A is selected from

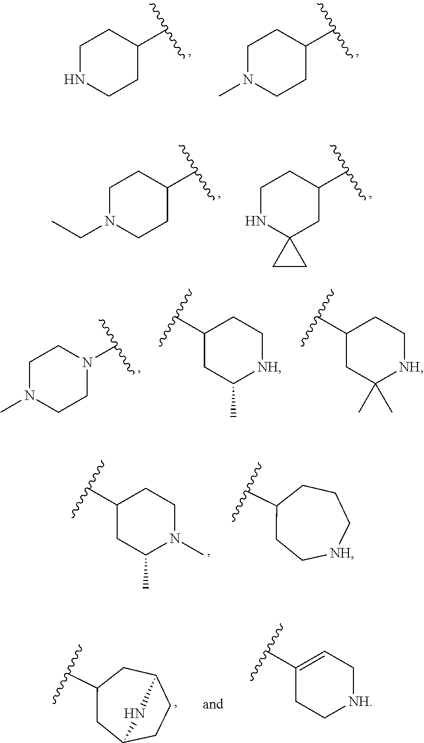

36. The compound of any one of claims 23-35, wherein each of L¹ and L² is independently absent.

37. The compound of any one of claims 23-36, wherein W is C(R³) (e.g., CH).

38. The compound of any one of claims 23-37, wherein Xis C(R³) (e.g., CH).

39. The compound of any one of claims 23-38, wherein Z is C(R³) (e.g., CH).

40. The compound of any one of claims 23-39, wherein Y is N(R^{4a}) or C(R^{4b}).

41. The compound of any one of claims 23-40, wherein Y is NH.

42. The compound of any one of the preceding claims, wherein R² is absent.

43. The compound of any one of claims 23-42, wherein the compound of Formula (I) is a compound of Formula (I-a): 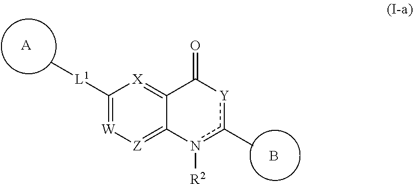
or a pharmaceutically acceptable salt, solvate, hydrate, tautomer, or stereoisomer thereof, wherein A, B, L¹, W, X, Z, R^{4a}, and subvariables thereof are as defined in claim 1.

44. The compound of any one of claims 23-43, wherein the compound of Formula (I) is a compound of Formula (I-b): 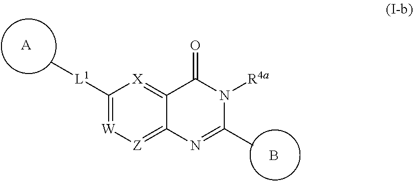
or a pharmaceutically acceptable salt, solvate, hydrate, tautomer, or stereoisomer thereof, wherein A, B, L¹, W, X, Z, R^{4a}, and subvariables thereof are as defined in claim 1.

45. The compound of any one of claims 23-44, wherein the compound of Formula (I) is a compound of Formula (I-c): 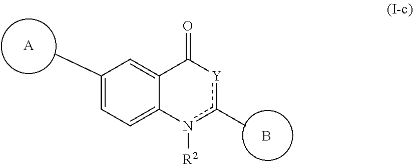
or a pharmaceutically acceptable salt, solvate, hydrate, tautomer, or stereoisomer thereof, wherein A, B, Y, R², and subvariables thereof are as defined in claim 1.

46. The compound of any one of the preceding claims, wherein the compound of Formula (I) is a compound listed in Table 1 or a pharmaceutically acceptable salt, solvate, hydrate, tautomer, or stereoisomer thereof.

47. A compound of Formula (III): 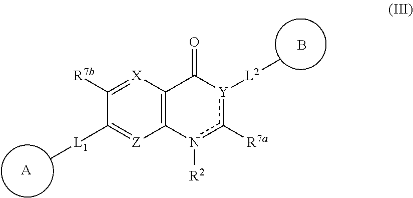
or a pharmaceutically acceptable salt, solvate, hydrate, tautomer, or stereoisomer thereof, wherein:
A and B are each independently cycloalkyl, heterocyclyl, aryl, or heteroaryl, each of which is optionally substituted with one or more R¹;
each of L¹ and L² is independently absent, C₁-C₆-alkylene, C₁-C₆-heteroalkylene, —O—, —C(O)—, —N(R⁸)—, —N(R⁸)C(O)—, or —C(O)N(R⁸)—, wherein each alkylene and heteroalkylene is optionally substituted with one or more R⁹;
each of X and Z is independently C(R³) or N;
Y is N, C, or C(R^{4b}), wherein the dashed lines in the ring comprising Y may be single or double bonds as valency permits;
each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D} —NO₂, —C(O)NR^{B}R^{C}, —C(O)R^{D}, C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁵; or
two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵;
R² is absent, hydrogen, or C₁-C₆-alkyl;
R³ is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —C(O)R^{D}, or —C(O)OR^{D};
R^{4b} is hydrogen, C₁-C₆-alkyl, C₁-C₆-heteroalkyl, or C₁-C₆-haloalkyl;
each R⁵ is independently C₁-C₆-alkyl, C₂-C₆-alkenyl, C₂-C₆-alkynyl, C₁-C₆₁-heteroalkyl, C₁-C₆-haloalkyl, cycloalkyl, heterocyclyl, aryl, heteroaryl, halo, oxo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁶;
each R⁶ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A};
R^{7a} is hydrogen, C₁-C₆-alkyl, C₁-C₆-heteroalkyl, C₁-C₆-haloalkyl, halo, cyano, oxo, or —OR^{A};
R^{7b} is hydrogen, C₁-C₆-alkyl, C₁-C₆-heteroalkyl, C₁-C₆-haloalkyl, halo, cyano, or —OR^{A};
each R⁸ is independently hydrogen, C₁-C₆-alkyl, or C₁-C₆-haloalkyl;
each R⁹ is independently C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D};
each R^{A}is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D};
each R^{B} and R^{C} is independently hydrogen, C₁-C₆ alkyl, C₁-C₆ heteroalkyl, cycloalkyl, heterocyclyl, —OR^{A}; or
R^{B} and R^{C} together with the atom to which they are attached form a 3-7-membered heterocyclyl ring optionally substituted with one or more R¹⁰;
each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl;
each R¹⁰ is independently C₁-C₆-alkyl or halo; and
x is 0, 1, or 2.

48. The compound of claim 47, wherein A is heterocyclyl or heteroaryl.

49. The compound of any one of claims 47-48, wherein A is a nitrogen-containing heterocyclyl or nitrogen-containing heteroaryl.

50. The compound of any one of claims 47-49, wherein A is selected from 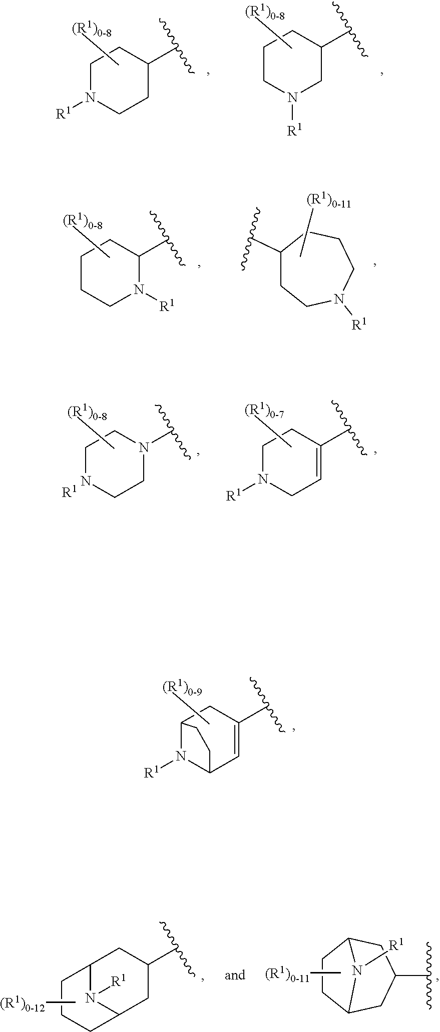
wherein R¹ is as described in claim 47.

51. The compound of claim 50, wherein A is selected from

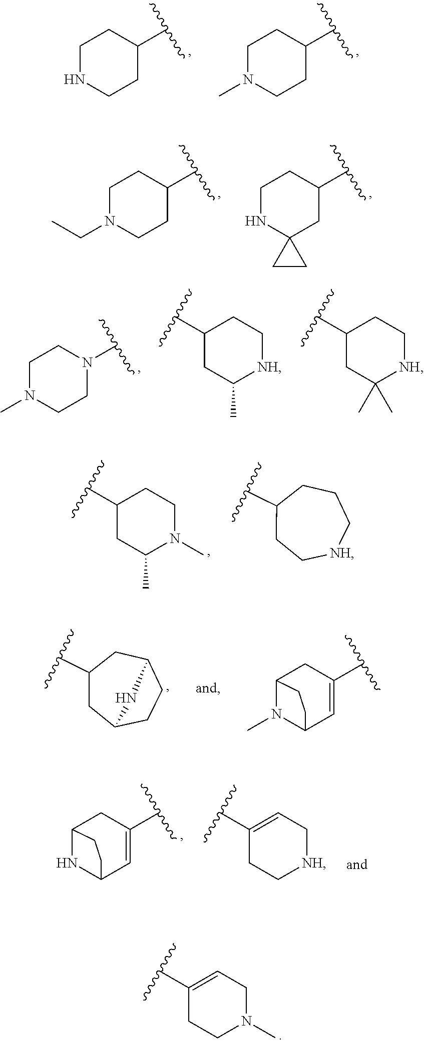

52. The compound of any one of claims 47-49, wherein A selected from 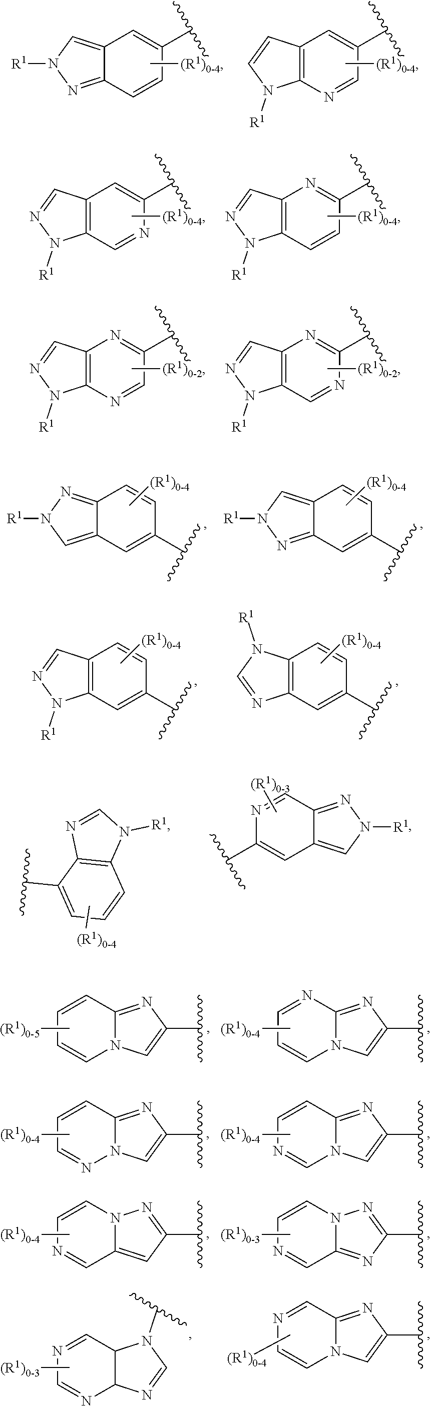 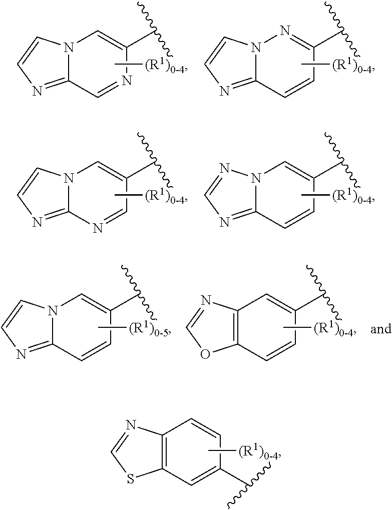
wherein R¹ is as described in claim 47.

53. The compound of claim 52, wherein A is selected from

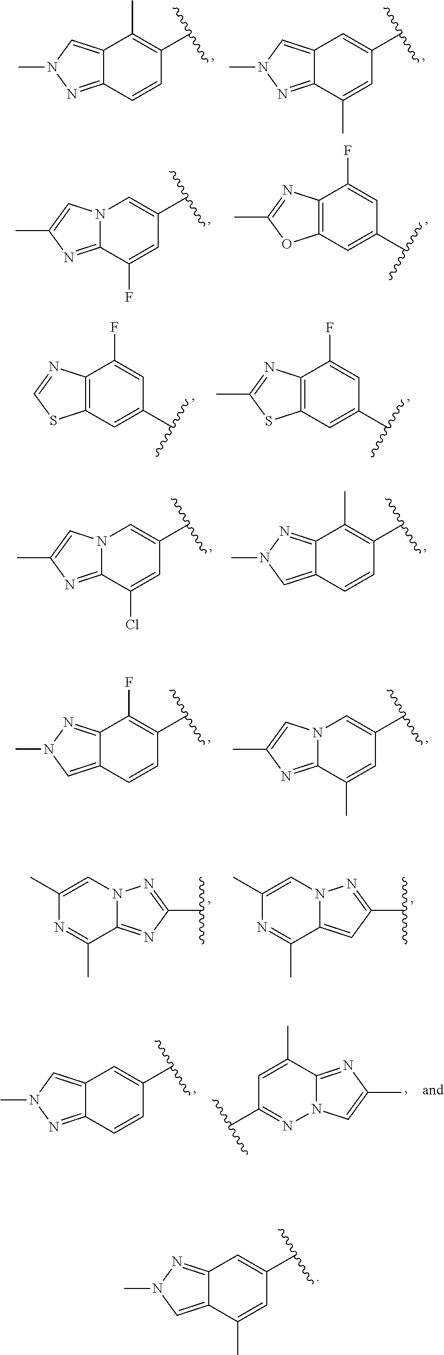

54. The compound of any one of claims 47-53, wherein B is a heteroaryl or heterocyclyl.

55. The compound of any one of claims 47-54, wherein B is a nitrogen-containing heteroaryl or nitrogen-containing heterocyclyl.

56. The compound of any one of claims 47-55, wherein B selected from 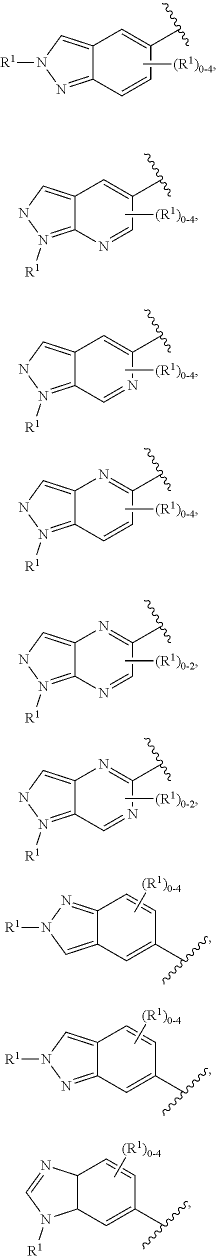 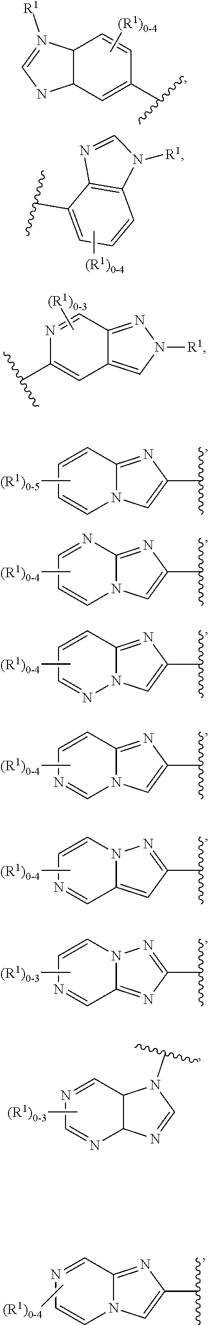 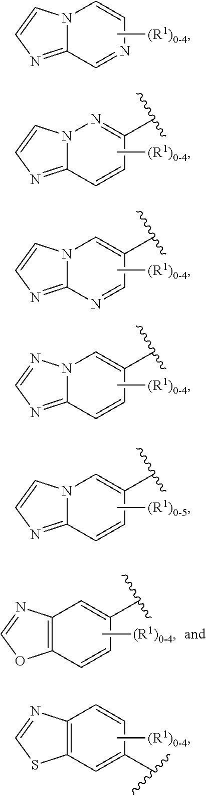
wherein R¹ is as described in claim 47.

57. The compound of claim 56, wherein B is selected from

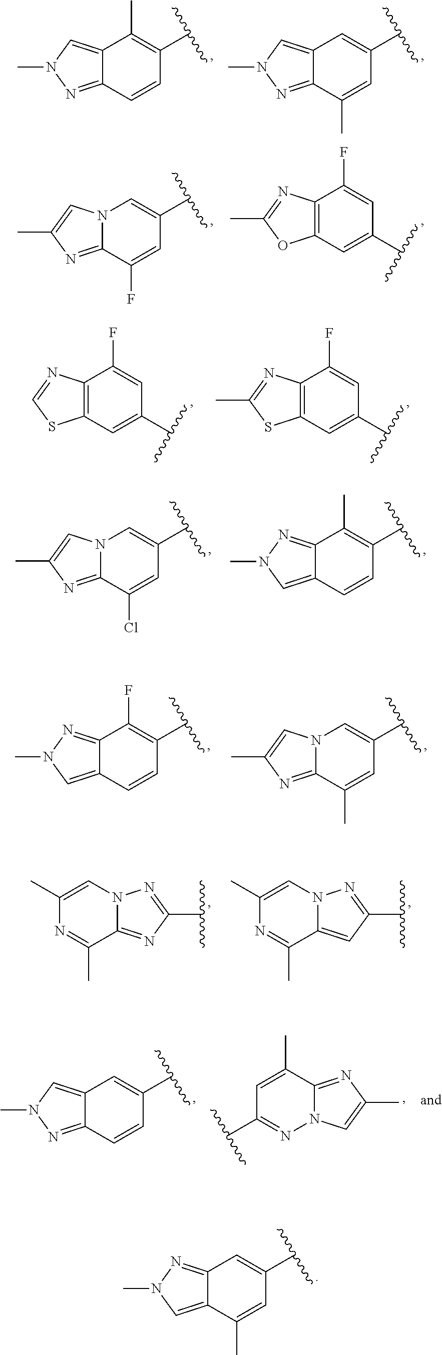

58. The compound of any one of claims 47-55, wherein B is selected from 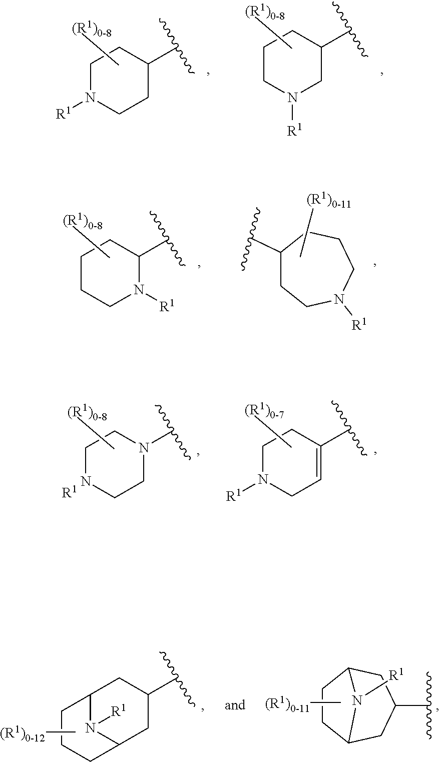
wherein R¹ is as described in claim 47.

59. The compound of claim 58, wherein B is selected from

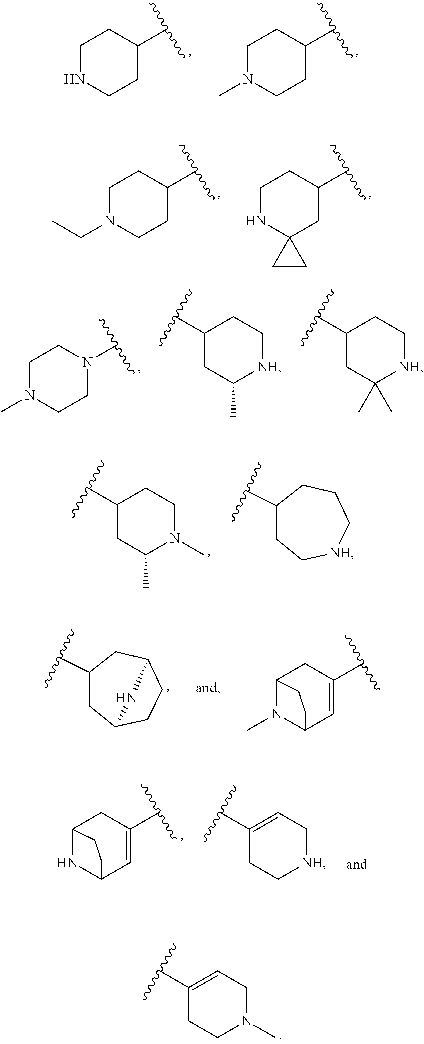

60. The compound of any one of claims 47-59, wherein each of L¹ and L² is independently absent.

61. The compound of any one of claims 47-60, wherein Xis C(R³) (e.g., CH).

62. The compound of any one of claims 47-61, wherein Z is C(R³) (e.g., CH).

63. The compound of any one of claims 47-62, wherein Y is N or C(R^{4b}).

64. The compound of any one of claims 47-63, wherein Y is N.

65. The compound of any one of claims 47-64, wherein R² is absent.

66. The compound of any one of claims 47-65, wherein each of R^{7a} and R^{7b} is independently hydrogen.

67. The compound of any one of claims 47-66, wherein the compound of Formula (III) is a compound of Formula (III-a): 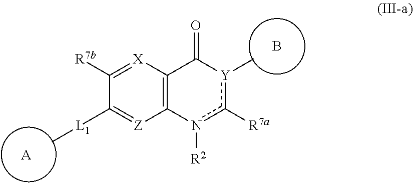
or a pharmaceutically acceptable salt, solvate, hydrate, tautomer, or stereoisomer thereof, wherein A, B, L¹, X, Z, R^{7a} , R^{7b}, and subvariables thereof are as defined in claim 47.

68. The compound of any one of claims 47-67, wherein the compound of Formula (III) is a compound of Formula (III-b): 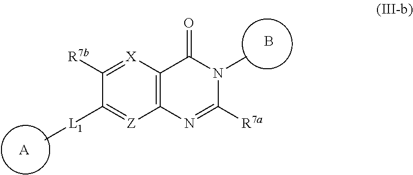
or a pharmaceutically acceptable salt, solvate, hydrate, tautomer, or stereoisomer thereof, wherein A, B, L¹, X, Z, R^{7a} , R^{7b}, and subvariables thereof are as defined in claim 47.

69. The compound of any one of claims 47-68, wherein the compound of Formula (III) is a compound of Formula (III-c): 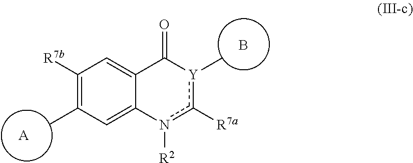
or a pharmaceutically acceptable salt, solvate, hydrate, tautomer, or stereoisomer thereof, wherein A, B, Y, R², R^{7a} , R^{7b}, and subvariables thereof are as defined in claim 47.

70. The compound of any one of claims 47-69, wherein the compound of Formula (III) is a compound listed in Table 3 or a pharmaceutically acceptable salt, solvate, hydrate, tautomer, or stereoisomer thereof.

71. A pharmaceutical composition comprising a compound of any one of claims 1-70 and a pharmaceutically acceptable excipient.

72. The compound of any one of claims 1-70 or the pharmaceutical composition of claim 71, wherein the compound alters a target nucleic acid (e.g., an RNA, e.g., a pre-mRNA).

73. The compound of any one of claims 1-70 or the pharmaceutical composition of claim 71, wherein the compound binds to a target nucleic acid (e.g., an RNA, e.g., a pre-mRNA).

74. The compound of any one of claims 1-70 or the pharmaceutical composition of claim 71, wherein the compound stabilizes a target nucleic acid (e.g., an RNA, e.g., a pre-mRNA).

75. The compound of any one of claims 1-70 or the pharmaceutical composition of claim 71, wherein the compound increases splicing at splice site on a target nucleic acid (e.g., an RNA, e.g., a pre-mRNA), by about 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or more, e.g., as determined by qPCR.

76. The compound of any one of claims 1-70 or the pharmaceutical composition of claim 71, wherein the compound decreases splicing at splice site on a target nucleic acid (e.g., an RNA, e.g., a pre-mRNA), by about 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or more, e.g., as determined by qPCR %.

77. A method of modulating splicing of a nucleic acid (e.g., DNA, RNA, e.g., a pre-mRNA) comprising contacting the nucleic acid with a compound of Formula (I), Formula (III), or Formula (V), according to any one of claims 1-70 or the pharmaceutical composition of claim

71.

78. The method of claim 77, wherein the compound increases splicing at splice site on a target nucleic acid (e.g., an RNA, e.g., a pre-mRNA), by about 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or more, e.g., as determined by qPCR.

79. The method of claim 77, wherein the compound decreases splicing at splice site on a target nucleic acid (e.g., an RNA, e.g., a pre-mRNA), by about 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or more, e.g., as determined by qPCR.

80. A method of forming a complex comprising a component of a spliceosome (e.g., a major spliceosome component or a minor spliceosome component), a nucleic acid (e.g., a DNA, RNA, e.g., a pre-mRNA), and a compound of Formula (I), Formula (III), or Formula (V):
comprising contacting the nucleic acid (e.g., a DNA, RNA, e.g., a pre-mRNA) with a compound of Formula (I), Formula (III), or Formula (V), according to any one of claims 1-70 or the pharmaceutical composition of claim 71.

81. The method of claim 80, wherein the component of a spliceosome is recruited to the nucleic acid in the presence of the compound of Formula (I), Formula (III), or Formula (V).

82. A method of altering the conformation of a nucleic acid (e.g., a DNA, RNA, e.g., a pre-mRNA) comprising contacting the nucleic acid with a compound of Formula (I), (III), or (V), according to any one of claims 1-70 or the pharmaceutical composition of claim 71.

83. The method of claim 82, wherein the altering comprises forming a bulge in the nucleic acid.

84. The method of claim 82, wherein the altering comprises stabilizing a bulge in the nucleic acid.

85. The method of claim 82, wherein the altering comprises reducing a bulge in the nucleic acid.

86. The method of any one of claims 82-85, wherein the nucleic acid comprises a splice site.

87. A method of treating a disease or disorder in a subject comprising administering to the subject a compound of Formula (I), Formula (III), or Formula (V) according to any one of claims 1-70 or the pharmaceutical composition of claim 71.

88. The method of claim 87, wherein the disease or disorder comprises a proliferative disease (e.g., cancer, a benign neoplasm, or angiogenesis).

89. The method of any one of claims 87-88, wherein the disease or disorder comprises a non-proliferative disease (e.g., a neurological disease, autoimmune disorder, immunodeficiency disorder, lysosomal storage disease, cardiovascular condition, metabolic disorder, respiratory condition, renal disease, or infectious disease).
